(19) United States
(12) Patent Application Publication
(10) Pub. No.: US 2024/0390418 A1
(43) Pub. Date: Nov. 28, 2024

(54) ANTI-VARIABLE MUC1* ANTIBODIES AND USES THEREOF

(71) Applicant: Minerva Biotechnologies Corporation, Waltham, MA (US)

(72) Inventors: Cynthia BAMDAD, Waltham, MA (US); Benoit SMAGGHE, Waltham, MA (US)

(21) Appl. No.: 18/018,536

(22) PCT Filed: Jul. 27, 2021
(86) PCT No.: PCT/US2021/071017

§ 371 Date: Jan. 27, 2023

Related U.S. Application Data
(60) Provisional application No. 63/184,724, filed on May 5, 2021.
(60) Provisional application No. 63/173,083, filed on Apr. 9, 2021.
(60) Provisional application No. 63/151,380, filed on Feb. 19, 2021.
(60) Provisional application No. 63/122,234, filed on Dec. 7, 2020.
(60) Provisional application No. 60/058,044, filed on Sep. 4, 1997.

(51) Int. Cl. A61K 35/17; A61K 39/00; A61P 35/00; C07K 14/725; C07K 16/30; C12N 5/0783; C12N 15/86

(52) U.S. Cl. CPC A61K 35/17; A61K 39/4611; A61K 39/4631; A61K 39/46447; A61P 35/00; C07K 14/7051; C07K 16/3092; C12N 5/0636; C12N 15/86; A61K 2239/13; A61K 2239/17; A61K 2239/21; A61K 2239/22; C07K 2317/565; C07K 2317/622; C07K 2317/73; C07K 2319/02; C07K 2319/03; C07K 2319/33; C12N 2510/00; C12N 2740/15043

(57) ABSTRACT

The present application discloses an antibody, or fragment thereof, for the diagnosis, treatment or prevention of cancers wherein the antibody specifically binds to the PSMGFR peptide (SEQ ID NO:2) or a fragment thereof of the peptide.

### SEQUENCE LISTING

The instant application contains a Sequence Listing which has been submitted electronically in ASCII format and is hereby incorporated by reference in its entirety. Said ASCII copy, created on Aug. 30, 2023, is named 56699-746_831 Replacement SL.txt and is 1,205,559 bytes in size.

### CROSS REFERENCE

This application is a national stage entry of International Application No. PCT/US2021/071017, filed Jul. 27, 2021, which claims the benefit of U.S. Provisional Application No. 63/184,724, filed May 5, 2021, U.S. Provisional Application No. 63/173,083, filed Apr. 9, 2021, U.S. Provisional Application No. 63/151,380, filed Feb. 19, 2021, U.S. Provisional Application No. 63/122,234, filed Dec. 7, 2020, and U.S. Provisional Application No. 63/058,044, filed Jul. 29, 2020, each of which is incorporated herein by reference it its entirety.

### BACKGROUND OF THE INVENTION

### 1. Field of the Invention

The present application relates to human, humanized and non-human anti-MUC1* antibodies and methods of making and using them. The present application also relates to using an immune cell transfected or transduced with a cleavage enzyme for the treatment of cancer. The present invention also relates to using an immune cell transfected or transduced with a CAR and another protein for the treatment of cancer.

### 2. General Background and State of the Art

We previously discovered that a cleaved form of the MUC1 (SEQ ID NO: 1) transmembrane protein is a growth factor receptor that drives the growth of over 75% of all human cancers. The cleaved form of MUC1, which we called MUC1* (pronounced muk 1 star), is a powerful growth factor receptor. Cleavage and release of the bulk of the extracellular domain of MUC1 unmasks a binding site for activating ligands dimeric NME1, NME6, NME7, NME7_{AB}, NME7-X1 or NME8. It is an ideal target for cancer drugs as it is aberrantly expressed on over 75% of all cancers and is likely overexpressed on an even higher percentage of metastatic cancers (Mahanta et al. (2008) A Minimal Fragment of MUC1 Mediates Growth of Cancer Cells. PLoS ONE 3(4): e2054. doi:10.1371/journal.pone.0002054; Fessler et al. (2009), “MUC1* is a determinant of trastuzumab (Herceptin) resistance in breast cancer cells,” Breast Cancer Res Treat. 118(1):113-124). After MUC1 cleavage most of its extracellular domain is shed from the cell surface. The remaining portion has a truncated extracellular domain that comprises most or all of the primary growth factor receptor sequence called PSMGFR (SEQ ID NO:2).

Antibodies are increasingly used to treat human diseases. Antibodies generated in non-human species have historically been used as therapeutics in humans, such as horse antibodies. More recently, antibodies are engineered or selected so that they contain mostly, or all, human sequences in order to avoid a generalized rejection of the foreign antibody. The process of engineering recognition fragments of a non-human antibody into a human antibody is generally called ‘humanizing’. The amount of non-human sequences that are used to replace the human antibody sequences determines whether they are called chimeric, humanized or fully human.

Alternative technologies exist that enable generation of humanized or fully human antibodies. These strategies involve screening libraries of human antibodies or antibody fragments and identifying those that bind to the target antigen, rather than immunizing an animal with the antigen. Another approach is to engineer the variable region(s) of an antibody into an antibody-like molecule. Another approach involves immunizing a humanized animal. The present invention is intended to also encompass these approaches for use with recognition fragments of antibodies that the inventors have determined bind to the extracellular domain of MUC1*.

In addition to treating patients with an antibody, cancer immunotherapies have recently been shown to be effective in the treatment of blood cancers. One cancer immunotherapy, called CAR T (chimeric antigen receptor T cell) therapy, engineers a T cell so that it expresses a chimeric receptor having an extra cellular domain that recognizes a tumor antigen, a transmembrane domain and cytoplasmic tail comprising T cell signaling and co-stimulatory components (Dai H, Wang Y, Lu X, Han W. (2016) Chimeric Antigen Receptors Modified T cells for Cancer Therapy. J Natl Cancer Inst. 108(7): djv439). Such receptor is composed of a single chain antibody fragment (scFv) that recognizes a tumor antigen, linked to a T cell transmembrane, signaling domain and co-stimulatory domain or domains. Upon binding of the receptor to a cancer associated antigen, a signal is transmitted resulting in T cell activation, propagation and the targeted killing of the cancer cells. In practice, T cells are isolated from a patient or donor and transduced with a CAR, expanded and then injected back into the patient. If from a donor, the immune cells may be mutated or engineered such that they do not induce graft versus host disease in the recipient. When the CAR T cells bind to the antigen on a cancer cell, the CAR T cells attack the cancer cells and then expand that population of T cells.

Thus far, CAR T therapies have been very successful in the treatment of blood cancers but as yet have not shown efficacy against solid tumors in humans. Because most blood cancers are B cell malignancies, the CAR T cells can just eliminate all of the patient's B cells without causing serious harm to the patient. There is no B cell equivalent in solid tumors. Most tumor associated antigens are also expressed on normal tissues; they are just expressed at a higher level in cancerous tissues. Thus, the challenge is to develop an antibody that recognizes an epitope on a tumor associated antigen that is somehow different in the context of the tumor compared to normal tissue. To further minimize the risk of off-tumor/on-target killing of normal tissues, the antibody should recognize and bind to cancerous tissues at least two-times more than normal tissues. Antibodies that are not so cancer selective may be used therapeutically if they are inducibly expressed at the tumor site.

Another cancer therapy that incorporates cancer selective antibodies is Bispecific T cell Engagers, also called BiTEs. The BiTE approach attempts to eliminate the CAR T associated risk of off-tumor/on-target effects. Unlike CAR T, BiTEs are bispecific antibodies that should not pose any greater risk than regular antibody-based therapies. However, unlike typical anti-cancer antibodies that bind to and block a cancer antigen, BiTEs are designed to bind to an antigen on the tumor cell and simultaneously bind to an antigen on an immune cell, such as a T cell. In this way, a BiTE recruits the T cell to the tumor. BiTEs are engineered proteins that simultaneously bind to a cancer associated antigen and a T cell surface protein such as CD3-epsilon. BiTEs are antibodies made by genetically linking the scFv's of an antibody that binds to a T cell antigen, like anti-CD3-epsilon to a scFv of a therapeutic monoclonal antibody that binds to a cancer antigen (Patrick A. Baeuerle, and Carsten Reinhardt (2009) Bispecific T cell engaging antibodies for cancer therapy. Cancer Res. 69(12):4941-4944). A drawback of BiTE technology is that, unlike CAR T cells, they do not expand in the patient, so have limited persistence.

Yet another cancer therapy that incorporates cancer selective antibodies is antibody drug conjugate, also called ADC, technology. In this case, a toxin, or a precursor to a toxin, is linked to a cancer selective antibody. Unlike CAR T cells that use the CD8 positive T cell's natural killing to kill cancer cells, ADCs carry a toxic payload to the tumor. Drawbacks of ADCs include the potential of delivering the toxic payload to normal cells and that most ADCs require binding to a cell surface molecule which then gets internalized after binding, with an approximate 10,000 surface molecule required for resultant cell death.

### SUMMARY OF THE INVENTION

In one aspect, the present invention is directed to a non-human, human or humanized anti-MUC1* antibody or antibody fragment or antibody-like protein that binds to a region on extracellular domain of MUC1 isoform or cleavage product that is devoid of the tandem repeat domains. The non-human, human or humanized anti-MUC1* antibody or antibody fragment or antibody-like protein may specifically bind to
(i) PSMGFR region of MUC1;
(ii) PSMGFR peptide;
(iii) a peptide having amino acid sequence of QFNQYKTEAASRYNLTISDVSVSDVPFPFSAQSGA (N-10) (SEQ ID NO:3)
(iv) a peptide having amino acid sequence of
ASRYNLTISDVSVSDVPFPFSAQSGA (N-19) (SEQ ID NO:4)
(v) a peptide having amino acid sequence of
NLTISDVSVSDVPFPFSAQSGA (N-23) (SEQ ID NO:5)
(vi) a peptide having amino acid sequence of
ISDVSVSDVPFPFSAQSGA (N-26) (SEQ ID NO:6)
(vii) a peptide having amino acid sequence of
SVSDVPFPFSAQSGA (N-30) (SEQ ID NO:7)
(viii) a peptide having amino acid sequence of
QFNQYKTEAASRYNLTISDVSVSDVPFPFS (N-10/C-5) (SEQ ID NO:8)
(ix) a peptide having amino acid sequence of
ASRYNLTISDVSVSDVPFPFS (N-19/C-5) (SEQ ID NO:9)
(x) a peptide having amino acid sequence of
FPFSAQSGA (SEQ ID NO:10)

The non-human, human or humanized antibody may be IgG1, IgG2, IgG3, IgG4 or IgM. The human or humanized antibody fragment or antibody-like protein may be scFv or scFv-Fc.

The murine, camelid, human or humanized antibody, antibody fragment or antibody-like protein as in above may comprise a heavy chain variable region and light chain variable region which is derived from mouse monoclonal MNC2, MNE6, 20A10, 3C2B1, 5C6F3, 25E6, 18G12, 28F9, 1E4, B12, B2, B7, B9, 8C7F3, and H11 antibody, and has at least 80%, 90% or 95% or 98% sequence identity to the mouse monoclonal MNC2, MNE6, 20A10, 3C2B1, 5C6F3, 25E6, 18G12, 28F9, 1E4, B12, B2, B7, B9, 8C7F3, and H11 antibody. The heavy chain variable region of CDR1 and CDR2 may have at least 90% or 95% or 98% sequence identity to the particularly indicated antibody heavy chain variable region sequence set forth in the present application in the sequence listing, and the light chain variable region of CDR1 and CDR2 may have at least 90% or 95% or 98% sequence identity to the particularly indicated antibody heavy chain variable region sequence set forth in the present application in the sequence listing section. The heavy chain variable region of CDR3 may have at least 80% or 85% or 90% sequence identity to the particularly indicated antibody heavy chain variable region sequence set forth in the present application in the sequence listing, and the light chain variable region of CDR3 may have at least 80% or 85% or 90% sequence identity to the particularly indicated antibody heavy chain variable region sequence set forth in the present application in the sequence listing section.

The murine, camelid, human or humanized antibody, antibody fragment or antibody-like protein according to above may include complementarity determining regions (CDRs) in the heavy chain variable region and light chain variable region having at least 90% or 95% or 98% sequence identity to the particularly indicated antibody heavy chain CDR1, CDR2 or CDR3 region and light chain CDR1, CDR2 or CDR3 region sequences set forth in the present application in the sequence listing section.

In another aspect, the present invention is directed to an anti-MUC1* extracellular domain antibody or anti-N-10 antibody, which may be any of the antibodies described above, comprised of sequences represented by humanized IgG2 heavy chain, or humanized IgG1 heavy chain, paired with humanized Kappa light chain, or humanized Lambda light chain. The humanized IgG2 heavy chain may be SEQ ID NOS:55, humanized IgG1 heavy chain may be SEQ ID NO:58, humanized Kappa light chain may be SEQ ID NO:110, and humanized Lambda light chain may be SEQ ID NO:114, or a sequence having 90%, 95% or 98% sequence identity thereof.

In another aspect, the present invention is directed to an anti-MUC1* extracellular domain antibody or anti-N-10 antibody, which may be any of the antibodies described above, comprised of sequences represented by human IgG2 heavy chain, or human IgG1 heavy chain, paired with human Kappa light chain, or human Lambda light chain. The human IgG2 heavy chain may be SEQ ID NOS:55, human IgG1 heavy chain may be SEQ ID NO:58, human Kappa light chain may be SEQ ID NO:110, and human Lambda light chain may be SEQ ID NO: 114, or a sequence having 90%, 95% or 98% sequence identity thereof.

In another aspect, the invention is directed to an anti-MUC1* extracellular domain antibody or anti-N-10 antibody comprised of sequences of a humanized MNC2 represented by humanized IgG1 heavy chain, humanized IgG2 heavy chain, paired with humanized Lambda light chain, and humanized Kappa light chain.

In another aspect, the invention is directed to an anti-MUC1* extracellular domain antibody or anti-N-10 antibody comprised of sequences of a humanized MNC2, MNE6, 20A10, 3C2B1, 5C6F3, 25E6, 18G12, 28F9, 1E4, B12, B2, B7, B9, 8C7F3, or H11 represented by humanized IgG1 heavy chain or humanized IgG2 heavy chain, paired with humanized Lambda light chain, or humanized Kappa light chain.

In another aspect, the invention is directed to an antibody that is “like” MNC2, MNE6, 20A10, 3C2B1, 5C6F3, 25E6, 18G12, 28F9, 1E4, B12, B2, B7, B9, 8C7F3, or H11 in that they have the same or very similar pattern of binding to subsets of peptides derived from the PSMGFR peptide, also do not recognize a linear epitope, competitively inhibit the binding of NME1 or NME7_{AB} to MUC1*, recognize a MUC1 transmembrane cleavage product produced by cleavage by MMP9 or contain CDR sequences that are at least 80% homologous to the MNE6, MNC2, MN18G12, MN20A10, MN25E6, MN28F9, MN5C6F3, MN3C2B1, and MN1E4 CDR consensus sequences.

In another aspect, the invention is directed to an antibody that binds to the extra cellular domain of a MUC1 that is devoid of the tandem repeat domain, which may be a cleavage product. In one aspect of the invention, the antibody binds to a peptide having the sequence of QFNQYKTEAASRYNLTISDVSVSDVPFPFSAQSGA (N-10). In one aspect of the invention, the antibody binds to a peptide having the sequence of ASRYNLTISDVSVSDVPFPFSAQSGA (N-19). In one aspect of the invention, the antibody binds to a peptide having the sequence of SVSDVPFPFSAQSGA (N-30). In one aspect of the invention, the antibody binds to a peptide having the sequence of FPFSAQSGA (N-36). Examples of such antibodies include but are not limited to monoclonal antibodies MNC2, MNE6, 20A10, 3C2B1, 5C6F3, 25E6, 18G12, 28F9, 1E4, B12, B2, B7, B9, 8C7F3, and H11. The heavy chain and light chain complementary determining region sequences for these antibodies are set forth in the present application in the sequence listing section.

In one aspect of the invention, one or more of these antibodies is administered to a patient diagnosed with or at risk of developing a cancer. The antibody may be human or humanized. The antibody may be murine or camelid. The antibody may be bivalent or monovalent. The antibody may be a fragment, including a single chain fragment, scFv, of one of the antibodies. The antibody or antibody fragment may be administered directly to the patient or incorporated into a multi-specific antibody-like molecule, a bispecific antibody, a bispecific T cell engager, BiTE, or an antibody drug conjugate, ADC. The antibody or antibody fragment may be incorporated into a T cell receptor, TCR. The sequence of the antibody or antibody fragment may be incorporated into a chimeric antigen receptor, a “CAR”, or other similar entity, then introduced into an immune cell, ex vivo, then administered to a patient diagnosed with or at risk of developing a cancer. The immune cell, which may be a T cell or natural killer cell, may be derived from a donor or from the patient. In one aspect the immune cell is derived from a stem cell that has been directed to differentiate to that immune cell type in vitro. In another aspect the immune cell is derived from a stem cell that has been directed to differentiate to that immune cell type in vitro. In another aspect, a CAR containing sequences of the antibody are expressed in a stem cell, which then may be differentiated into an immune cell. In one case, the immune cell is a T cell. In another case, the immune cell is an NK cell. In one aspect, the antibody or a CAR containing sequences of the antibody may be expressed off of an inducible promoter. In one case the antibody or the CAR is expressed upon activation of the T cell or other immune cell. In one instance, the antibody or the CAR of the invention is expressed off of an NFAT response element. In another instance, CAR recognition of a target tumor cell activates the immune cell, leading to NFAT inducible expression of a cytokine, such as IL-12 or IL-18, or expression of a checkpoint inhibitor such as a PD1 inhibitor or a PDL-1 inhibitor. In yet another aspect, CAR recognition of a target tumor cell activates the immune cell, leading to NFAT inducible expression of a second CAR that contains sequences of a second antibody.

In another aspect, the invention is directed to a murine, camelid, human, humanized anti-MUC1* antibody or antibody fragment or antibody-like protein that binds to the N-10 peptide, according to above, which inhibits the binding of NME protein to MUC1*. The NME may be NME1, NME6, NME7_{AB}, NME7-X1, NME7 or NME8.

In yet another aspect, the invention is directed to a single chain variable fragment (scFv) comprising a heavy and light chain variable regions connected via a linker, further comprising CDRs of antibodies that bind to MUC1* extracellular domain. The CDRs may be derived from MNC2, MNE6, 20A10, 3C2B1, 5C6F3, 25E6, 18G12, 28F9, 1E4, B12, B2, B7, B9, 8C7F3, and H11. The scFv may be one that possesses the SEQ ID NOS:233, 235 or 237 (MNE6); SEQ ID NOS:238-243, 654-655 or 5017-5020 (MNC2); SEQ ID NOS:1574-1581 or 5001-5012 (20A10); SEQ ID NOS:1573 or 1813 (3C2B1); SEQ ID NOS:1385 or 1815 (5C6F3); SEQ ID NOS:1599 or 1601 (25E6).

In still another aspect, the invention is directed to a chimeric antigen receptor (CAR) comprising a scFv or a humanized variable region that binds to the extracellular domain of a MUC1 that is devoid of tandem repeats, a linker molecule, a transmembrane domain and a cytoplasmic domain. The single chain antibody fragment may bind to
(i) PSMGFR region of MUC1;
(ii) PSMGFR peptide;
(iii) a peptide having amino acid sequence of QFNQYKTEAASRYNLTISDVSVSDVPFPFSAQSGA (N-10) (SEQ ID NO:3)
(iv) a peptide having amino acid sequence of
ASRYNLTISDVSVSDVPFPFSAQSGA (N-19) (SEQ ID NO:4)
(v) a peptide having amino acid sequence of
NLTISDVSVSDVPFPFSAQSGA (N-23) (SEQ ID NO:5)
(vi) a peptide having amino acid sequence of
ISDVSVSDVPFPFSAQSGA (N-26) (SEQ ID NO:6)
(vii) a peptide having amino acid sequence of
SVSDVPFPFSAQSGA (N-30) (SEQ ID NO:7)
(viii) a peptide having amino acid sequence of
QFNQYKTEAASRYNLTISDVSVSDVPFPFS (N-10/C-5) (SEQ ID NO:8)
(ix) a peptide having amino acid sequence of
ASRYNLTISDVSVSDVPFPFS (N-19/C-5) (SEQ ID NO:9)
(x) a peptide having amino acid sequence of
FPFSAQSGA (N-36) (SEQ ID NO: 10)

In the CAR as described above, portions of any of the variable regions set forth and described above, or combination thereof may be used in the extracellular domain of the CAR. The CAR also comprises a transmembrane region and a cytoplasmic tail that comprises sequence motifs that signal immune system activation. The extracellular domain may be comprised of murine, camelid, human, non-human, or humanized single chain antibody fragments of an MNC2, MNE6, 20A10, 3C2B1, 5C6F3, 25E6, 18G12, 28F9, 1E4, B12, B2, B7, B9, 8C7F3, and H11. Additional antibodies from which single chain antibody fragments may be made include but are not limited to monoclonal antibodies that are like MNC2, MNE6, 20A10, 3C2B1, 5C6F3, 25E6, 18G12, 28F9, 1E4, B12, B2, B7, B9, 8C7F3, and H11 in that they have the same or very similar pattern of binding to subsets of peptides derived from the PSMGFR peptide, may not recognize a linear epitope or competitively inhibit the binding of NME1 or NME7_{AB} to MUC1*, or recognize a MUC1 transmembrane cleavage product produced by cleavage by MMP9 or contain CDR sequences that are at least 80% homologous to the MNC2, MNE6, 20A10, 3C2B1, 5C6F3, 25E6, 18G12, 28F9, 1E4, B12, B2, B7, B9, 8C7F3, and H11 CDR consensus sequences.

In the CARs as described above, the extracellular domain may include a murine, camelid, human, non-human or humanized single chain antibody fragments of an MNE6 scFv set forth as SEQ ID NOS: 233, 235, or 237, MNC2 scFv (SEQ ID NOS:239, 241,243, 655 or 5017-5020), 20A10 scFv as set forth as SEQ ID NOS:1575, 1577, 1579, 1581 or 5001-5012, 3C2B1 scFv as set forth as SEQ ID NOS:1573 or 1813, 5C6F3 scFv as set forth as SEQ ID NOS:1385 or 1815, or 25E6 scFv as set forth as SEQ ID NOS:1599 or 1601.

In the process of humanizing an antibody, one must annotate the sequence to identify the different functional regions, such as the complementarity determining regions (CDRs), the framework regions and the constant regions. Various computer programs are available that assign certain sequences to the CDRs, framework regions and constant regions. Depending on the program used, the exact position where one region ends and the next begins may differ by a few amino acids. Typically, humanized single chain constructs, scFvs, contain heavy and light chain CDRs supported by intervening framework regions, wherein the heavy and light chain are connected through a flexible linker. Depending on the annotation program used, the sequence assigned to framework region IV may extend into the constant region. In some cases, extension of framework region IV may provide more stability to the scFv. Here we provide sequences for humanized scFv's of the invention, wherein the length of framework region IV of the light chain may vary. For example, in some cases the C-terminus of Framework region IV ends in the amino acids R, T. In other cases, it ends in R alone. Still in other cases, the terminal R and T are both omitted. In the CARs described here, the extracellular domain may include a murine, camelid, human, non-human or humanized single chain antibody fragments with framework region IV having variable lengths as set forth as MNE6 scFv (SEQ ID NOS: 5014 or 5016), MNC2 scFv (SEQ ID NOS: 5018 or 5020), or 20A10 scFv (SEQ ID NOS: 5002, 5004, 5006, 5008, 5010 or 5012) or 25E6 scFv (SEQ ID NOS: 5030 or 5032).

In any of the CARs described here, the cytoplasmic tail may be comprised of one or more of signaling sequence motifs and co-stimulatory domains, including but not limited to CD3-zeta, CD3-zeta-1XX CD27, CD28, 4-1BB, OX40, CD30, CD40, ICAm-1, LFA-1, ICOS, CD2, CD5, or CD7. Additionally, the sequence of the intracellular signaling domain may contain mutations that dampen the signal to improve persistence or to improve killing of low antigen density tumor cells. The cytoplasmic tail may be comprised of one or more of signaling sequence motifs and co-stimulatory sequence motifs CD3-zeta, CD27, CD28, 4-1BB, OX40, CD30, CD40, ICAm-1, LFA-1, ICOS, CD2, CD5, or CD7. The transmembrane and extracellular hinge region of the CAR may or may not be derived from sequences of the adjacent co-stimulatory domain. For example, a CAR comprising the 4-1BB co-stimulatory domain may have a transmembrane and hinge region derived from CD8 or CD28. In another example, a CAR comprising the CD28 co-stimulatory domain may have a transmembrane and hinge region derived from CD28. In any of the CARs described above, the cytoplasmic tails may include deletions or mutations that dampen signaling. Such deletions or mutations in one or more of the three immunoreceptor tyrosine-based activation motifs, also known as ITAMs, increase persistence of CAR bearing cells and decrease their differentiation as measured by an increase in the CD62L+ CD45RA− population. Such mutations include but are not limited to Tyrosines that are mutated to inhibit phosphorylation and signaling (Salter et al, 2018). In another aspect one or two ITAMs are deleted, leaving only one or two ITAMs (Feucht et al 2019). In another aspect, the position of the included ITAM or ITAMs is moved to a position proximal to the co-stimulatory domain. Suitable ITAM configurations for increased persistence of CARs include but are not limited to 1XX, X2X, XX3, 12X and 23X, wherein the numeral 1, 2 or 3 refers to ITAM1, ITAM2, or ITAM3 and X refers to the deletion of that ITAM. In a preferred embodiment ITAM1 is the only functional ITAM included in the CAR construct, also known as 1XX. In any of the CARs described above, the ITAMs of CD3-zeta may be deleted or mutated to inhibit or dampen signaling. In any of the CARs described above, the CD3 of the cytoplasmic tail may comprise deletions or mutations in or of the ITAMs including those referred to as 1XX (Feucht et al 2019; SEQ ID NO:1796-1797). In any of the CARs described above, the T cell may be engineered to overexpress c-Jun as a method to inhibit T cell exhaustion (Lynn et al 2019). The CAR constructs described above may be expressed in a T cell, an NK cell, a dendritic cell or other immune cell, which may be autologous or allogeneic. Allogeneic cells may be derived from human stem cells.

In any of the CARs described above, the CAR may include a single chain antibody fragment, scFv comprising a sequence derived from antibody MNE6, including but not limited to (SEQ ID NOS:12-13 and 65-66, 56-57, 107-108, 341-342, 391-394), from antibody MNC2, including but not limited to (SEQ ID NOS:118-119 and 168-169, 144-145 and 194-195, 654-655, 1788-1789), from antibody 20A10, including but not limited to (SEQ ID NOS:988-989 and 1004-1005, 1574-1581, 5001-5012, 1677, 1687), from antibody 3C2B1, including but not limited to (SEQ ID NOS:1820-1823, 1572-1573, 1812-1813), from antibody 5C6F3, including but not limited to (SEQ ID NOS:1816-1819, 1384-1385, 1814-1815), from antibody 25E6, including but not limited to (SEQ ID NOS:1020-1021, 1036-1037, 1598-1601), wherein the CAR hinge and transmembrane sequences may be derived from CD8 (SEQ ID NO:346 and SEQ ID NO:364), or from CD28 (SEQ ID NO:350 and SEQ ID NO:368), further comprising a co-stimulatory domain, which may be 41BB (SEQ ID NO:659) or CD28 (SEQ ID NO:378) and the CD3-zeta signaling domain may be derived from (SEQ ID NO:661) or may contain mutations including those referred to as 1XX (SEQ ID NO:1796-1797).

In any of the CARs described above, the sequence may be CAR MNE6 CD28/CD3z (SEQ ID NOS:298); CAR MNE6 4-1BB/CD3z (SEQ ID NOS:301); CAR MNE6 OX40/CD3z (SEQ ID NOS:617); CAR MNE6 CD28/4-1BB/CD3z (SEQ ID NOS:304); CAR MNE6 CD28/OX40/CD3z (SEQ ID NOS:619); CAR MNC2 CD3z (SEQ ID NOS:607); CAR MNC2 CD8 hinge/transmembrane CD28/CD3z SEQ ID NOS:609); CAR MNC2 CD8 hinge/transmembrane 4-1BB/CD3z (SEQ ID NOS:611 and SEQ ID NOS: 719); CAR MNC2 CD8 hinge/transmembrane 4-1BB/1XX (SEQ ID NOS:1619 and SEQ ID NOS: 1621); CAR MNC2 CD8 hinge/transmembrane CD28/1XX (SEQ ID NOS:1623 and SEQ ID NOS: 1625); CAR MNC2 CD28 hinge/transmembrane CD28/1XX (SEQ ID NO:5042 and SEQ ID NO: 5044); CAR MNC2 OX40/CD3z (SEQ ID NOS:613); CAR MNC2 CD28/4-1BB/CD3z (SEQ ID NOS: 307); CAR MNC2 CD28/OX40/CD3z (SEQ ID NOS:615); CAR 20A10 CD8 hinge/transmembrane 4-1BB/CD3z (SEQ ID NO:1583 and SEQ ID NO: 1585); CAR 20A10 CD8 hinge/transmembrane CD28/CD3z (SEQ ID NO:1587 and SEQ ID NO: 1589); CAR 20A10 CD8 hinge/transmembrane 4-1BB/1XX (SEQ ID NO:1591 and SEQ ID NO:1593); CAR 20A10 CD8 hinge/transmembrane CD28/1XX (SEQ ID NO:1595 and SEQ ID NO: 1597); CAR 20A10 CD28 hinge/transmembrane CD28/CD3z (SEQ ID NO:5022 and SEQ ID NO:5024); CAR 20A10 CD28 hinge/transmembrane CD28/1XX (SEQ ID NO:5026 and SEQ ID NO:5028); or CAR MNC3 4-1BB/CD3z (SEQ ID NOS: 601).

In another aspect, the invention is directed to a composition that includes at least two CARs with different extracellular domain units transfected into the same cell, which may be an immune cell, which may be derived from the patient requiring treatment for a cancer. The expression of the second CAR may be inducible and driven by the recognition of a target by the first CAR. The nucleic acid encoding the second CAR may be linked to an inducible promoter. The expression of the second CAR may be induced by an event that occurs specifically when the immune cell mounts an immune response to a target tumor cell. The antibody fragments of one or both of the CARs may direct the cell to a MUC1* positive tumor. The antibody fragments of the first and second CARs may bind to a MUC1* that is produced when MUC1 is cleaved by two different cleavage enzymes. Expression of the second CAR by the inducible promoter may be induced when the antibody fragment of the first CAR engages or binds to a MUC1 or MUC1* on the tumor. One way to do this is to induce expression of the second CAR when, or shortly after, an NFAT protein is expressed or translocated to the nucleus. For example, a sequence derived from an NFAT promoter region is put upstream of the gene for the second CAR. In this way, when the transcription factors that bind to the promoter of the NFAT protein are present in sufficient concentration to bind to and induce transcription of the NFAT protein, they will also bind to that same promoter that is engineered in front of the sequence for transcription of the second CAR. The NFAT protein may be NFAT1 also known as NFATc2, NFAT2 also known as NFATc or NFATc1, NFAT3 also known as NFATc4, NFAT4 also known as NFATc3, or NFAT5. In one aspect of the invention, the NFAT is NFATc1, NFATc3 or NFATc2. In one aspect of the invention, the NFAT is NFAT2 also known as NFATc1. SEQ ID NO:646 shows nucleic acid sequence of the upstream transcriptional regulatory region for NFAT2. The recognition unit of the second CAR may be an antibody fragment or a peptide, wherein the recognition units may bind to NME7, PD-1, PDL-1, or a checkpoint inhibitor.

The at least two CARs may have one CAR that does not have a tumor antigen targeting recognition unit and the other CAR does have a tumor antigen targeting recognition unit. In another aspect of the invention, one of the extracellular domain recognition units may bind to MUC1* extracellular domain. In another aspect of the invention, one of the extracellular domain recognition units may be an antibody fragment and the other is a peptide, which may be devoid of transmembrane and signaling motifs; the peptide may be a single chain antibody fragment or antibody. In another aspect of the invention, one of the recognition units may bind PD-1 or PDL-1. In another aspect of the invention, one extra cellular domain recognition unit is an anti-MUC1* antibody, antibody fragment or scFv chosen from the group consisting of MNC2, MNE6, 20A10, 3C2B1, 5C6F3, 25E6, 18G12, 28F9, 1E4, B12, B2, B7, B9, 8C7F3, and H11. The other recognition unit may be a CAR or may be an anti-NME7 antibody.

In another aspect, the invention is directed to a cell comprising a CAR with an extracellular domain that binds to the extra cellular domain of a MUC1 molecule that is devoid of tandem repeats. In another aspect, the invention is directed to a cell comprising a CAR with an extracellular domain that binds to a MUC1* transfected or transduced cell. The cell that includes the CAR may be an immune system cell, preferably a T cell, a natural killer cell (NK), a dendritic cell or mast cell.

In another aspect, the invention is directed to an engineered antibody-like protein.

In another aspect, the invention is directed to a method for treating a disease in a subject comprising administering an antibody according to any claim above, to a person suffering from the disease, wherein the subject expresses MUC1 aberrantly. The disease may be cancer, such as breast cancer, ovarian cancer, pancreatic cancer, lung cancer, colon cancer, gastric cancer or esophageal cancer.

In another aspect, the invention is directed to an antibody, antibody fragment or scFv comprising variable domain fragments derived from an antibody that binds to an extracellular domain of MUC1 isoform or cleavage product that is devoid of the tandem repeat domains. In a preferred embodiment, the antibody or antibody fragment binds to the N-10 peptide (SEQ ID NO:3), but does not bind to the C-10 peptide (SEQ ID NO:825). The variable domain fragments may be derived from mouse monoclonal antibody MNE6 (SEQ ID NO:13 and 66) or from the humanized MNE6 (SEQ ID NO: 39 and 94), or from MNE6 scFv (SEQ ID NO: 233, 235 and 237). Or, the variable domain fragments may be derived from mouse monoclonal antibody MNC2 (SEQ ID NO: 119 and 169) or from the humanized MNC2 (SEQ ID NO: 145 and 195), or from MNC2 scFv (SEQ ID NO: 239, 241 and 243). Or, the variable domain may be derived from monoclonal antibodies MN18G12, MN20A10, MN25E6, MN28F9, MN5C6F3, MN3C2B1, or MN1E4. The heavy chain and light chain complementary determining region sequences for these antibodies are also set forth in the sequence listing herein.

In another aspect, the invention is directed to a method for the treatment of a person diagnosed with, suspected of having or at risk of developing a MUC1 or MUC1* positive cancer involving administering to the person an effective amount of the antibody, antibody fragment or scFv described above, wherein the species may be murine, camelid, human or humanized.

In another aspect, the invention is directed to a polypeptide comprising at least two different scFv sequences, wherein one of the scFv sequences is a sequence that binds to extracellular domain of MUC1 isoform or cleavage product that is devoid of the tandem repeat domains. The polypeptide may bind to
(i) PSMGFR region of MUC1;
(ii) PSMGFR peptide;
(iii) a peptide having amino acid sequence of QFNQYKTEAASRYNLTISDVSVSDVPFPFSAQSGA (N-10) (SEQ ID NO:3)
(iv) a peptide having amino acid sequence of
ASRYNLTISDVSVSDVPFPFSAQSGA (N-19) (SEQ ID NO:4)
(v) a peptide having amino acid sequence of
NLTISDVSVSDVPFPFSAQSGA (N-23) (SEQ ID NO:5)
(vi) a peptide having amino acid sequence of
ISDVSVSDVPFPFSAQSGA (N-26) (SEQ ID NO:6)
(vii) a peptide having amino acid sequence of
SVSDVPFPFSAQSGA (N-30) (SEQ ID NO:7)
(viii) a peptide having amino acid sequence of
QFNQYKTEAASRYNLTISDVSVSDVPFPFS (N-10/C-5) (SEQ ID NO:8)
(ix) a peptide having amino acid sequence of
ASRYNLTISDVSVSDVPFPFS (N-19/C-5) (SEQ ID NO:9)
(x) a peptide having amino acid sequence of
FPFSAQSGA (N-36) (SEQ ID NO:10)

In another aspect, the invention is directed to a method of detecting presence of a cell that expresses MUC1* aberrantly, comprising contacting a sample of cells or tissue with the antibody, antibody fragment or scFv-Fc described above and detecting for the presence of the binding of antibody, antibody fragment or scFv-Fc to the cell. The cell may be cancer cell.

In another aspect, the invention is directed to a method for testing a subject's cancer for suitability of treatment with a composition comprising antibodies of the invention, which may be murine, camelid, human or humanized, or fragments thereof, or portions of the variable regions of antibodies MNC2, MNE6, 20A10, 3C2B1, 5C6F3, 25E6, 18G12, 28F9, 1E4, B12, B2, B7, B9, 8C7F3, or H11, comprising the steps of contacting a bodily specimen from the patient, in vitro, ex-vivo, or in vivo, with the antibody and determining that the patient exhibits aberrant expression of MUC1* compared to normal tissue or specimen and concluding that the patient's cancer will beneficially respond to treatment with an agent comprising the antibody or a fragment thereof. The antibody used in these diagnostics may be conjugated to an imaging agent.

In another aspect, the invention is directed to a method of treating a subject suffering from a disease comprising, exposing immune cells, which may be T cells or NK cells from the subject, or from a donor, to MUC1* peptides wherein through various rounds of maturation, the T cells or NK cells develop MUC1* specific receptors, creating adapted T cells or NK cells, and expanding and administering the adapted cells to the donor patient who is diagnosed with, suspected of having, or is at risk of developing a MUC1* positive cancer. The MUC1* peptide is chosen from among the group:
(i) PSMGFR region of MUC1;
(ii) PSMGFR peptide;
(iii) a peptide having amino acid sequence of QFNQYKTEAASRYNLTISDVSVSDVPFPFSAQSGA (N-10)
(iv) a peptide having amino acid sequence of
ASRYNLTISDVSVSDVPFPFSAQSGA (N-19)
(v) a peptide having amino acid sequence of
NLTISDVSVSDVPFPFSAQSGA (N-23)
(vi) a peptide having amino acid sequence of
ISDVSVSDVPFPFSAQSGA (N-26)
(vii) a peptide having amino acid sequence of
SVSDVPFPFSAQSGA (N-30)
(viii) a peptide having amino acid sequence of
QFNQYKTEAASRYNLTISDVSVSDVPFPFS (N-10/C-5)
(ix) a peptide having amino acid sequence of
ASRYNLTISDVSVSDVPFPFS (N-19/C-5)
(x) a peptide having amino acid sequence of
FPFSAQSGA (N-36)

In one aspect of the invention, the antibody that is administered to a patient for the treatment or prevention of a MUC1 or MUC1* positive cancer is selected for its ability to bind to the N-10 peptide of the PSMGFR. The antibody can be administered alone, as a monovalent antibody, as an scFv, or a fragment of the antibody can be incorporated into a CAR, a BiTE or an ADC.

In another aspect of the invention, the antibody that is administered to a patient for the treatment or prevention of a MUC1 or MUC1* positive cancer is selected for its ability to bind to the QFNQYKTEAASRYNLTISDVSVSDVPFPFSAQSGA (N-10) peptide, wherein the presence of the FPFSAQSGA (N-36) sequence is required for binding. The antibody can be administered alone, as a monovalent antibody, as an scFv, or a fragment of the antibody can be incorporated into a CAR, a BiTE or an ADC.

In one aspect of the invention, the antibody that is administered to a patient for the treatment or prevention of a MUC1 or MUC1* positive cancer is selected for its inability to recognize a linear epitope of MUC1 or MUC1*. The antibody can be administered alone, as a monovalent antibody, as an scFv, or a fragment of the antibody can be incorporated into a CAR, a BiTE or an ADC.

In one aspect of the invention, the antibody that is administered to a patient for the treatment or prevention of a MUC1 or MUC1* positive cancer is selected for its ability to recognize the MUC1 transmembrane cleavage product after it has been cleaved by MMP9. The antibody can be administered alone, as an antibody, a monovalent antibody, as an scFv, a bispecific antibody, a multi-specific antibody, or a fragment of the antibody can be incorporated into a BiTE, an ADC, or a CAR which can be expressed in an immune cell.

In one aspect of the invention, the antibody that is administered to a patient for the treatment or prevention of a MUC1 or MUC1* positive cancer is selected for its ability to competitively inhibit the binding of NME7_{AB} or NME7-X1 to the extra cellular domain of a MUC1 that is devoid of tandem repeats. The antibody can be administered alone, as an antibody, a monovalent antibody, as an scFv, a bispecific antibody, a multi-specific antibody, or a fragment of the antibody can be incorporated into a BiTE, an ADC, or a CAR which can be expressed in an immune cell.

In another aspect, the invention is directed to a method of treating cancer in a patient comprising administering to the patient the antibody, antibody fragment, BiTE, ADC or CAR expressed in an immune cell of any of the above, in combination with a checkpoint inhibitor.

In the method above, any of the antibodies, or variable regions thereof, set forth in the following may be used: MNC2, MNE6, 20A10, 3C2B1, 5C6F3, 25E6, 18G12, 28F9, 1E4, B12, B2, B7, B9, 8C7F3, or H11.

In the method above, any of the variable regions set forth in the following may be used:
(i) an anti-MUC1* extracellular domain antibody or anti-N-10 antibody comprised of sequences of a humanized MN-E6 represented by humanized IgG2 heavy chain, or humanized IgG1 heavy chain, paired with humanized Kappa light chain, or humanized Lambda light chain;
(ii) an antibody of (i), wherein the humanized IgG2 heavy chain is SEQ ID NOS:53, humanized IgG1 heavy chain is SEQ ID NO:57, humanized Kappa light chain is SEQ ID NO:108, and humanized Lambda light chain is SEQ ID NO:112, or a sequence having 90%, 95% or 98% sequence identity thereof;
(iii) an anti-MUC1* extracellular domain antibody or anti-N-10 antibody comprised of sequences of a humanized MN-C2 represented by humanized IgG1 heavy chain, humanized IgG2 heavy chain, paired with humanized Lambda light chain, and humanized Kappa light chain;
(iv) an antibody of (iii), wherein the humanized IgG1 heavy chain MN-C2 (SEQ ID NOS:159) or IgG2 heavy chain (SEQ ID NOS:164) paired with Lambda light chain (SEQ ID NO:219) or Kappa light chain (SEQ ID NO:213), or a sequence having 90%, 95% or 98% sequence identity thereof;
In the method above, in the CAR, the extracellular domain may be comprised of humanized single chain antibody fragments of MNC2, MNE6, 20A10, 3C2B1, 5C6F3, 25E6, 18G12, 28F9, 1E4, B12, B2, B7, B9, 8C7F3, or H11. The extracellular domain may be comprised of humanized single chain antibody fragments of an MN-E6 scFv set forth as SEQ ID NOS: 233, 235, or 237), MN-C2 scFv (SEQ ID NOS:239, 241, or 243). In the CAR, the cytoplasmic tail may be comprised of one or more of signaling sequence motifs and co-stimulatory domains, including but not limited to CD3-zeta-1XX, CD27, CD28, 4-1BB, OX40, CD30, CD40, ICAm-1, LFA-1, ICOS, CD2, CD5, or CD7 and CD3-zeta or variants 1XX, X2X, XX3, 12X, or 23X. Additionally, the sequence of the intracellular signaling domain may contain mutations that dampen the signal to improve persistence or target cell killing.

The method above may include two CARs with different extracellular domain units transfected into the same cell. One of the extracellular domain recognition units may bind to MUC1* extracellular domain. One of the extracellular domain recognition units may bind to PD-1. One of the extracellular domain recognition units may be an antibody fragment and the other may be a peptide or an anti-MUC1* antibody fragment.

The method may include an immune cell transfected or transduced with a plasmid encoding a CAR and a plasmid encoding a non-CAR species that is expressed from an inducible promoter. The non-CAR species may be expressed from an inducible promoter that is activated by elements of an activated immune cell. The non-CAR species may be expressed from an NFAT inducible promoter. The NFAT may be NFATc1, NFATc3 or NFATc2. The cleavage enzyme may be MMP2, MMP3, MMP9, MMP13, MMP14, MMP16, ADAM10, ADAM17, or ADAM28, or a catalytically active fragment thereof. The non-CAR species may be a cytokine. The cytokine may be IL-7, IL-12, IL-15 or IL-18.

The present invention is directed to an antibody, or fragment thereof, for the diagnosis, treatment or prevention of cancers wherein the antibody specifically binds to the PSMGFR peptide (SEQ ID NO:2) or a fragment thereof of the peptide.

The antibody binds to the N-10 peptide (SEQ ID NO:3), N-19 peptide (SEQ ID NO:4), N-23 peptide (SEQ ID NO:5), N-26 peptide (SEQ ID NO:6), N-30 peptide (SEQ ID NO:7), N-10/C-5 peptide (SEQ ID NO:8), N-19/C-5 peptide (SEQ ID NO:9), or C-5 peptide (SEQ ID NO:825).

The antibody interacts with a peptide comprising conformational epitope SVSDV (SEQ ID NO:1751) and FPFSA (SEQ ID NO:1747) within N-26 sequence ISDVSVSDVPFPFSAQSGA (SEQ ID NO:6), wherein mutation or deletion of FPFS (SEQ ID NO:1747) destroys binding of the antibody or fragment thereof to the N-26 peptide.

The antibody interacts with a peptide comprising conformational epitope ASRYNLT (SEQ ID NO:1745), SVSDV (SEQ ID NO:1751), and FPFSA (SEQ ID NO:1747) within the N-19 sequence ASRYNLT ISDVSVSDVPFPFSAQSGA (SEQ ID NO:4), wherein mutation or deletion of ASRYNLT (SEQ ID NO:1745) destroys binding of the antibody or fragment thereof to the N-26 peptide.

The antibody does not bind to the C-10 peptide (SEQ ID NO:825).

The antibody binds to the N-10 peptide (SEQ ID NO:3), but not to the C-10 peptide (SEQ ID NO:825).

The antibody inhibits interaction between NME7_{AB} and MUC1*.

The antibody inhibits interaction between NME7_{AB} and PSMGFR peptide (SEQ ID NO:2).

The antibody inhibits interaction between NME7_{AB} and N-10 peptide (SEQ ID NO:3), N-19 peptide (SEQ ID NO:4), N-23 peptide (SEQ ID NO:5), N-26 peptide (SEQ ID NO:6), N-30 peptide (SEQ ID NO:7), N-10/C-5 peptide (SEQ ID NO:8), N-19/C-5 peptide (SEQ ID NO:9), or C-5 peptide (SEQ ID NO:825).

The antibody recognizes a MUC1 transmembrane enzymatic cleavage product.

In the above, the cleavage enzyme is MMP14 or MMP9 or a catalytically active fragment thereof of the enzyme.

The antibody binds to PSMGFR (SEQ ID NO:2) or fragment thereof in which presence of an amino acid sequence within PSMGFR (SEQ ID NO:2) induces binding of the antibody to the PSMGFR.

The amino acid sequence of the binding conformationally inducing peptide is present in N-10 peptide (SEQ ID NO:3).

The antibody does not bind to a linear form of the binding conformationally inducing peptide sequence wherein the linear form of the peptide is a denatured form.

The binding conformationally inducing peptide sequence is in the N-26 peptide sequence ISDVSVSDVPFPFSAQSGA (SEQ ID NO:6), wherein mutation or deletion of FPFS (SEQ ID NO:1747) destroys binding of the antibody or fragment thereof to the N-26 peptide.

The binding conformationally inducing peptide sequence is located within the N-19 sequence ASRYNLTISDVSVSDVPFPFSAQSGA (SEQ ID NO:4), wherein mutation or deletion of ASRYNLT (SEQ ID NO:1745) destroys binding of the antibody or fragment thereof to the N-19 peptide.

The binding inducing peptide sequence may be located within the N-26 sequence ISDVSVSDVPFPFSAQSGA (SEQ ID NO:6), wherein mutation or deletion within FPFS (SEQ ID NO:1747) destroys binding of the antibody or fragment thereof to PSMGFR.

The antibodies may have a consensus sequence.
heavy chain CDR1 comprises consensus sequence at least 90% identical to sequence: F or I at position 1, T at position 2, F at position 3, S at position 4, T, G, or R at position 5, Y at position 6, A, G or T at position 7, M at position 8 and S at position 9;
heavy Chain CDR2 comprises consensus sequence at least 90% identical to sequence: T at position 1, I or S at position 2, I or S at position 3, G or R at position 5, G or A at position 6, T or I at position 9, Y at position 10, Y at position 11, P or S at position12 and DSVKG for positions 13-17;
heavy chain CDR3 comprises consensus sequence at least 90% identical to sequence:_G, L, or N at position 2, G or T at position 4, Y at position 7, D or E at position 12, A at position 14, and Y at position 15;
light chain CDR1 comprises consensus sequence at least 90% identical to sequence: K or R at position 1, A or S at position 2, S at position 3, K or Q at position 4, S at position 5, L or V at position 6, L at position 7, T or S at position 10, Y at position 15, and I, L or M at position 16;
light Chain CDR2 comprises consensus sequence at least 90% identical to sequence: L or W, or S at position 1, A or T at position 2, S at position 3, N or T at position 4, L or R at position 5, E or A at position 6, and S at position 7; and
light chain CDR3 comprises consensus sequence at least 90% identical to sequence: Q at position 1, H or Q at position 2, S, Q or R at position 3, R, S or Y at position 4, E, L, or S at position 5, L or S at position 6, P or S at position 7, F or L at position 8 and T at position 9.

An antibody binding to a conformational epitope within a peptide having the N-26 sequence ISDVSVSDVPFPFSAQSGA (SEQ ID NO:6), wherein mutation or deletion within FPFS (SEQ ID NO: 1747), SVSDV (SEQ ID NO:1751), or ASRYNLT (SEQ ID NO: 1745) destroys binding of the antibody or fragment thereof to PSMGFR.

The antibody may have a further consensus sequence,
wherein
heavy chain CDR1 comprises consensus sequence at least 90% identical to sequence: F or I at position 1, T or A at position 2, F at position 3, S at position 4, T, G, or R at position 5, Y or F at position 6, A, G or T at position 7, M at position 8 and S at position 9;
heavy Chain CDR2 comprises consensus sequence at least 90% identical to sequence: T or A at position 1, I or S at position 2, I or S at position 3, N, S, T or G at position 4, G or R at position 5, G or A at position 6, G, T, or D at position 7, Y, K, H or S at position 8, T or I at position 9, Y or F at position 10, Y at position 11, P or S at position12 and D at position 13, S or T at position 14, V or L at position 15 and KG for positions 16-17;
heavy chain CDR3 comprises consensus sequence at least 90% identical to sequence: G, L, or N at position 2, G, T, or Y at position 3, G or T at position 4, Y at position 7, Y, A, or G at position 10, M, D or F at position 11, D or E at position 12 and AY at position 14-15;
light chain CDR1 comprises consensus sequence at least 90% identical to sequence: K or R at position 1, A or S at position 2, S or R at position 3, S, Y, I or V at position 8, T or S at position 10, G, S, D, or Q at position 12, V, Y, K or N at position 13, N, S, or T at position 14, Y or F at position 15, and I, L or M at position 16;
light Chain CDR2 comprises consensus sequence at least 90% identical to sequence: A, T or V at position 2, S at position 3, N, T, or K at position 4, L or R at position 5, E, A, F or D at position 6, and S at position 7; and
light chain CDR3 comprises consensus sequence at least 90% identical to sequence: Q, F or W at position 1, H or Q at position 2, R, S, T, Y or N at position 4, E, L, S or H at position 5, L, S, V, D or Y at position 6, P or S at position 7, and T at position 9.

The antibody above which may be MNC2, having
heavy chain CDR1 comprises consensus sequence FTFSGYAMS;
heavy Chain CDR2 comprises consensus sequence TISSGGTYIYYPDSVKG;
heavy chain CDR3 comprises consensus sequence -LGGDNYYEYFDV--;
light chain CDR1 comprises consensus sequence RASKS--VSTSGYSYMH;
light Chain CDR2 comprises consensus sequence LASNLES; and
light chain CDR3 comprises consensus sequence QHSRELPFT.
MNE6, having
heavy chain CDR1 comprises consensus sequence FTFSRYGMS;
heavy Chain CDR2 comprises consensus sequence TISGGGTYIYYPDSVKG;
heavy chain CDR3 comprises consensus sequence DNYGRNYDYGMDY--;
light chain CDR1 comprises consensus sequence -------SATSSVSYIH;
light Chain CDR2 comprises consensus sequence STSNLAS; and
light chain CDR3 comprises consensus sequence QQRSSSPFT.
B2, having
heavy chain CDR1 comprises consensus sequence FAFSTFAMS;
heavy Chain CDR2 comprises consensus sequence AISNGGGYTYYPDTLKG;
heavy chain CDR3 comprises consensus sequence ----RYYDLYFDL--;
light chain CDR1 comprises consensus sequence RSSQNIV-HSNGNTYLE;
light Chain CDR2 comprises consensus sequence KVSNRFS; and
light chain CDR3 comprises consensus sequence FQDSHVPLT.
B7, having
heavy chain CDR1 comprises consensus sequence FTFSRYGMS;
heavy Chain CDR2 comprises consensus sequence TISSGGTYIYYPDSVKG;
heavy chain CDR3 comprises consensus sequence DNYGSSYDYAMDY--;
light chain CDR1 comprises consensus sequence RSSQTIV-HSNGNTYLE;
light Chain CDR2 comprises consensus sequence KVSNRFS; and
light chain CDR3 comprises consensus sequence FQDSHVPLT.
B9, having
heavy chain CDR1 comprises consensus sequence FTFSRYGMS;
heavy Chain CDR2 comprises consensus sequence TISSGGTYIYYPDSVKG;
heavy chain CDR3 comprises consensus sequence DNYGSSYDYAMDY--;
light chain CDR1 comprises consensus sequence -------SASSSVSYMH;
light Chain CDR2 comprises consensus sequence TTSNLAS; and
light chain CDR3 comprises consensus sequence QQRSSYPF-.
8C7F3, having
heavy chain CDR1 comprises consensus sequence FTFSTYAMS;
heavy Chain CDR2 comprises consensus sequence AISNGGGYTYYPDSLKG;
heavy chain CDR3 comprises consensus sequence ----RYYDHYFDY--;
light chain CDR1 comprises consensus sequence --RASESVATYGNNFMQ;
light Chain CDR2 comprises consensus sequence LASTLDS; and
light chain CDR3 comprises consensus sequence QQNNEDPPT.
H11, having
heavy chain CDR1 comprises consensus sequence FAFSTFAMS;
heavy Chain CDR2 comprises consensus sequence AISNGGGYTYYPDTLKG;
heavy chain CDR3 comprises consensus sequence ----RYYDLYFDL--;
light chain CDR1 comprises consensus sequence RSSQNIV-HSNGNTYLE;
light Chain CDR2 comprises consensus sequence KVSNRFS; and
light chain CDR3 comprises consensus sequence FQDSHVPLT.
B12, having
heavy chain CDR1 comprises consensus sequence SYGVH;
heavy Chain CDR2 comprises consensus sequence VIWPGGSTNYNSTLMSRM;
heavy chain CDR3 comprises consensus sequence DRTPRVGAWFAY; and
light chain CDR1 comprises consensus sequence RASESVATYGNNFMQ;
light Chain CDR2 comprises consensus sequence LASTLDS; and
light chain CDR3 comprises consensus sequence QQNNEDPPT.
20A10, having
heavy chain CDR1 comprises consensus sequence FTFSTYAMS;
heavy Chain CDR2 comprises consensus sequence -SIGRAGSTYYSDSVKG;
heavy chain CDR3 comprises consensus sequence ---GPIYNDYDEFAY;
light chain CDR1 comprises consensus sequence KSSQSVLYSSNQKNYLA;
light Chain CDR2 comprises consensus sequence WASTRES; and
light chain CDR3 comprises consensus sequence HQYLSSLT.
3C2B1, having
heavy chain CDR1 comprises consensus sequence ITFSTYTMS;
heavy Chain CDR2 comprises consensus sequence TISTGGDKTYYSDSVKG;
heavy chain CDR3 comprises consensus sequence -GTTAMYYYAMDY;
light chain CDR1 comprises consensus sequence RASKS---ISTSDYNYIH;
light Chain CDR2 comprises consensus sequence LASNLES; and
light chain CDR3 comprises consensus sequence QHSRELPLT.

In another aspect, the invention is directed to an antibody, or fragment thereof, for the diagnosis, treatment or prevention of cancers that requires presence of antibody binding conformationally inducing peptide ASRYNLT (SEQ ID NO:1745) of PSMGFR (SEQ ID NO:2). The antibody may be 25E6, having
heavy chain CDR1 comprises consensus sequence FTFSSYGMS;
heavy Chain CDR2 comprises consensus sequence TISNGGRHTFYPDSVKG;
heavy chain CDR3 comprises consensus sequence QTGTEGWFAY;
light chain CDR1 comprises consensus sequence KSSQSLLDSDGKTYLN;
light Chain CDR2 comprises consensus sequence LVSKLDS_; and
light chain CDR3 comprises consensus sequence WQGTHFPQT.

In another aspect, the invention is directed to an antibody, or fragment thereof, for the diagnosis, treatment or prevention of cancers that requires presence of antibody binding conformationally inducing peptide SVSDV (SEQ ID NO:1761) of PSMGFR (SEQ ID NO:2). The antibody may be 5C6F3, having
heavy chain CDR1 comprises consensus sequence FTFSTYAMS;
heavy Chain CDR2 comprises consensus sequence AISNGGGYTYYPDSLKG;
heavy chain CDR3 comprises consensus sequence RYYDHYFDY;
light chain CDR1 comprises consensus sequence RSSQTIVHSNGNTYLE;
light Chain CDR2 comprises consensus sequence KVSNRFS; and
light chain CDR3 comprises consensus sequence FQDSHVPLT.

The antibody or fragment thereof according all of the above may be murine, camelid, human or humanized. The antibody fragment may be scFv or scFv-Fc, which variable regions thereof may be murine, camelid, human or humanized.

In another aspect, the invention is directed to a chimeric antigen receptor (CAR) comprising the antibody fragments of above, and may further comprise mutations in the co-stimulatory domain or mutations or deletions of one or two of the ITAMs of the CD3-zeta signaling domain. Tyrosines may be mutated in CD28 or 4-1BB. CD3-zeta may contain a single ITAM such as only ITAM1 also known as 1XX, ITAM2 also known as X2X, or ITAM3 also known as XX3. In another aspect, CD3-zeta may contain two ITAMs, wherein the positions of the ITAMs may be moved to a more proximal position such as 12X or 23X (Feucht et al 2019). In yet another aspect, tyrosines of one or two ITAMs may be mutated to dampen signaling. In a preferred embodiment, the CD3-zeta domain is 1XX. An example of 1XX mutations, includes those exemplified in SEQ ID NOS: 1796-1797.

In another aspect, the invention is directed to an immune cell comprising the CAR of above. Immune cell may be T cell, NK cell, dendritic cell, or mast cell. In one aspect the immune cell is derived from a stem cell that has been directed to differentiate to that immune cell type in vitro. In another aspect, a CAR containing sequences of the antibody are expressed in a stem cell, which then may be differentiated into an immune cell.

In another aspect, the invention is directed to a cell composition expressed in a cell comprising a CARs of above, and second entity having a biological recognition unit that has a specificity that is different from that of the CAR. The second entity may bind PD-1, PDL-1, or other checkpoint inhibitor, or NME7, or a cytokine such as IL-12 or IL-18, or c-Jun.

In yet another aspect, the invention is directed to an immune cell engineered to express a nucleic encoding a CAR of above and a nucleic acid encoding a second entity as in any of the claims above wherein the second entity expressed from an inducible promoter. The second entity may be expressed from an inducible promoter that is activated by elements of an activated immune cell. The second entity may be expressed from an NFAT inducible promoter. NFAT may be NFATcT, NFATc3 or NFATc2. The second entity may be a cytokine such as IL-7, IL-15, or IL-18. The nucleic acids encoding the second entity may be inserted into a Foxp3 promoter or enhancer region, wherein the cytokine is IL-18. The cytokine may be expressed from an NFAT inducible promoter.

In another aspect, the invention is directed to a BiTE construct comprising the antibody fragment of above.

In yet another aspect, the invention is directed to an antibody drug conjugate (ADC) comprising the antibody or antibody fragment of above.

The present invention is directed to an antibody or fragment thereof that specifically:
(i) binds to PSMGFR (SEQ ID NO:2) and N-10 (SEQ ID NO:3); and
does not bind to full-length MUC1;
(ii) does not bind to C-10 (SEQ ID NO:825);
(iii) competitively inhibits binding of NME1 or NME7_{AB} to MUC1* extra cellular domain or a PSMGFR peptide;
(iv) recognizes a MUC1* generated by cleavage by a cleavage enzyme;
(v) recognizes a conformational epitope and not a linear epitope; or
(vi) is cancer selective by immunohistochemistry on tissues.

Four of the criteria (i)-(vi) may be satisfied. Five of the criteria (i)-(vi) may be satisfied. Six of the criteria (i)-(vi) may be satisfied. At least criteria (vi) may be satisfied. Cleavage enzyme may be MMP-9.

In all of the above, the cancer may be breast cancer, pancreatic cancer, ovarian cancer, lung cancer, colon cancer, gastric cancer or esophageal cancer.

The present invention is also directed to a method of diagnosing, treating or preventing cancer by administering the antibodies and fragments disclosed herein to a cancer patient in need thereof that has been identified as expressing MUC1 aberrantly and expressing truncated MUC1, such as MUC1*.

These and other objects of the invention will be more fully understood from the following description of the invention, the referenced drawings attached hereto and the claims appended hereto.

### BRIEF DESCRIPTION OF THE DRAWINGS

The patent or application file contains at least one drawing executed in color. Copies of this patent or patent application publication with color drawings will be provided by the Office upon request and payment of the necessary fee.

The present invention will become more fully understood from the detailed description given herein below, and the accompanying drawings which are given by way of illustration only, and thus are not limitative of the present invention, and wherein;

FIGS. 1A-1D show cell growth assay graphs of MUC1* positive cells treated with either bivalent ‘by’ anti-MUC1* antibody, monovalent ‘my’ or Fab, NM23-H1 dimers or NME7-AB. Bivalent anti-MUC1* antibodies stimulate growth of cancer cells whereas the monovalent Fab inhibits growth (FIG. TA-1B). Classic bell-shaped curve indicates ligand induced dimerization stimulates growth. Dimeric NM23-H1, aka NME1, stimulates growth of MUC1* positive cancer cells but siRNA to suppress MUC1 expression eliminate its effect (FIG. 1C). NME7-AB also stimulates the growth of MUC1* positive cells (FIG. 1D).

FIGS. 2A-2I show results of ELISA assays. MUC1* peptides PSMGFR, PSMGFR minus 10 amino acids from the N-terminus aka N-10, or PSMGFR minus 10 amino acids from the C-terminus, aka C-10 are immobilized on the plate and the following are assayed for binding: NME7-AB (FIG. 2A), MNC2 monoclonal antibody (FIG. 2B), MNE6 monoclonal antibody (FIG. 2C), or dimeric NME1 (FIG. 2D). These assays show that NME1, NME7-AB and monoclonal antibodies MNC2 and MNE6 all require the first membrane proximal 10 amino acids of the MUC1* extracellular domain to bind. MUC1* peptides PSMGFR minus 10 amino acids from the N-terminus aka N-10, or PSMGFR minus 10 amino acids from the C-terminus, aka C-10, are immobilized on the plate and the following are assayed for binding: MNC3 (FIG. 2E) and MNC8 (FIG. 2F). FIG. 2G shows the amino acid sequence of the PSMGFR peptide. FIG. 2H shows the amino acid sequence of the N-10 peptide. FIG. 2I shows the amino acid sequence of the C-10 peptide.

FIGS. 3A-3C show results of competitive ELISA assays. The PSMGFR MUC1* peptide is immobilized on the plate and dimeric NM23-H1, aka NME1, is added either alone or after the MNE6 antibody has been added (FIG. 3A). The same experiment was performed wherein NM23-H7, NME7-AB, is added alone or after MNE6 has been added (FIG. 3B). Results show that MNE6 competitively inhibits the binding of MUC1* activating ligands NME1 and NME7. In a similar experiment (FIG. 3C), PSMGFR or PSMGFR minus 10 amino acids from the N-terminus, aka N-10, is immobilized on the plate. Dimeric NM23-H1 is then added. Anti-MUC1* antibodies MNE6, MNC2, MNC3 or MNC8 are then tested for their ability to compete off the NM23-H1. Results show that although all three antibodies bind to the PSMGFR peptides, MNE6 and MNC2 competitively inhibit binding of the MUC1* activating ligands.

FIGS. 4A-4F show FACS scans of anti-MUC1* antibody huMNC2scFv binding specifically to MUC1* positive cancer cells and MUC1* transfected cells but not MUC1* or MUC1 negative cells. ZR-75-1, aka 1500, MUC1* positive breast cancer cells were stained with 1:2 or 1:10 dilutions of the 1.5 ug/ml humanized MNC2. After two washes, cells were stained with secondary antibody, Anti-Penta-His antibody conjugated to Alexa 488 (Qiagen) dilutions of 1:200 (FIG. 4A), 1:50 (FIG. 4B), or 1:10 (FIG. 4C) to detect the 6× His tag on the huMNC2 scFv. FIG. 4A shows huMNC2 binding to ZR-75-1 breast cancer cells where secondary antibody is added at a 1:200 dilution. FIG. 4B shows huMNC2 binding to ZR-75-1 breast cancer cells where secondary antibody is added at a 1:50 dilution. FIG. 4C shows huMNC2 binding to ZR-75-1 breast cancer cells where secondary antibody is added at a 1:10 dilution. Flow cytometric analysis revealed a concentration-dependent shift of a subset of cells, indicating specific binding, which is unseen in the absence of the MNC2 scFv (FIG. 4A-4C). FIG. 4D shows anti-MUC1* antibody MNE6 staining of MUC1 negative HCT-116 colon cancer cells transfected with the empty vector, single cell clone #8. FIG. 4E shows anti-MUC1* antibody MNE6 staining of HCT-116 colon cancer cells transfected with MUC1* single cell clone #10. FIG. 4F shows anti-MUC1* antibody MNE6 staining of ZR-75-1, aka 1500, MUC1* positive breast cancer cells. As the FACS scans show, both MNC2 and MNE6 only stain MUC1* positive cells and not MUC1 or MUC1* negative cells.

FIG. 5 shows a graph of an ELISA in which surface is coated with either the MUC1* PSMGFR peptide or a control peptide. Humanized MNC2 scFv is then incubated with the surface, washed and detected according to standard methods. The ELISA shows that the huMNC2 scFv binds to the MUC1* peptide with an EC-50 of about 333 nM.

FIGS. 6A-6B show graphs of cancer cell growth inhibition by MUC1* antibody variable region fragment humanized MNC2 scFv. hMNC2 scFv potently inhibited the growth of ZR-75-1, aka 1500, MUC1* positive breast cancer cells (FIG. 6A) and T47D MUC1* positive breast cancer cells (FIG. 6B) with approximately the same EC-50 as the in vitro ELISAs.

FIGS. 7A-7B show graphs of tumor growth in immune compromised mice that have been implanted with human tumors then treated with anti-MUC1* antibody MNE6 Fab or mock treatment. Female nu/nu mice implanted with 90-day estrogen pellets were implanted with 6 million T47D human breast cancer cells that had been mixed 50/50 with Matrigel. Mice bearing tumors that were at least 150 mm³ and had three successive increases in tumor volume were selected for treatment. Animals were injected sub cutaneously twice per week with 80 mg/kg MNE6 Fab and an equal number of mice fitting the same selection criteria were injected with vehicle alone (FIG. 7A). Male NOD/SCID mice were implanted with 6 million DU-145 human prostate cancer cells that had been mixed 50/50 with Matrigel. Mice bearing tumors that were at least 150 mm³ and had three successive increases in tumor volume were selected for treatment. Animals were injected sub-cutaneously every 48 hours with 160 mg/kg MNE6 Fab and an equal number of mice fitting the same selection criteria were injected with vehicle alone (FIG. 7B). Tumors were measured independently by two researchers twice per week and recorded. Statistics were blindly calculated by independent statistician, giving a P value of 0.0001 for each. Anti-MUC1* Fab inhibited breast cancer growth and prostate cancer growth. Treatment had no effect on weight, bone marrow cell type or number.

FIG. 8 shows a graph of an ELISA wherein the surface was immobilized with either PSMGFR peptide, PSMGFR minus 10 amino acids from the N-terminus or minus 10 amino acids from the C-terminus. The huMNE6 scFv-Fc bound to the PSMGFR peptide and to the PSMGFR N-10 peptide but not to the PSMGFR C-10 peptide. The parent MNE6 antibody and the humanized MNE6 require the C-terminal 10 amino acids of PSMGFR for binding.

FIGS. 9A-9B show graphs of ELISAs wherein the assay plate surface was immobilized with either PSMGFR peptide, PSMGFR minus 10 amino acids from the N-terminus or minus 10 amino acids from the C-terminus. The MNC3 antibody variants were then assayed for binding to the various MUC1* peptides. FIG. 9A shows purified mouse monoclonal MNC3 antibody; and FIG. 9B shows the humanized MNC3 scFv-Fc. ELISAs show binding to the PSMGFR peptide as well as to certain deletion peptides.

FIGS. 10A-10J. FIG. 10A10B are photographs of breast cancer tissue arrays. FIG. 10A was stained with VU4H5 which recognizes MUC1-FL (full length); FIG. 10B was stained with mouse monoclonal antibody MNC2 which recognizes cancerous MUC1*. Following automated staining (Clarient Diagnostics), the tissue staining was scored using Allred scoring method which combines an intensity score and a distribution score. FIG. 10C10F are color coded graphs showing the score calculated for MUC1 full-length staining for each patient's tissue. FIG. 10G10J are color coded graphs showing the score calculated for MUC1* staining for each patient's tissue.

FIGS. 11A-11J. FIG. 11A11B are photographs of breast cancer tissue arrays. FIG. 11A was stained with VU4H5 which recognizes MUC1-FL (full length); FIG. 11B was stained with mouse monoclonal antibody MNC2 which recognizes cancerous MUC1*. Following automated staining (Clarient Diagnostics), the tissue staining was scored using Allred scoring method which combines an intensity score and a distribution score. FIGS. 11C-11F are color coded graphs showing the score calculated for MUC1 full-length staining for each patient's tissue. FIGS. 11G-11J are color coded graphs showing the score calculated for MUC1* staining for each patient's tissue.

FIGS. 12A-12H show photographs of normal breast and breast cancer tissues stained with humanized MNE6-scFv-Fc biotinylated anti-MUC1* antibody at 2.5 ug/mL, then stained with a secondary streptavidin HRP antibody. FIG. 12A is a normal breast tissue.

FIGS. 12B-12D are breast cancer tissues from patients as denoted in the figure. FIGS. 12E-12H are photographs of the corresponding serial sections that were stained with the secondary antibody alone.

FIGS. 13A-13F show photographs of normal breast and breast cancer tissues stained with humanized MNE6-scFv-Fc biotinylated anti-MUC1* antibody at 2.5 ug/mL, then stained with a secondary streptavidin HRP antibody. FIG. 13A is a normal breast tissue.

FIGS. 13B-13C are breast cancer tissues from patients as denoted in the figure. FIGS. 13D-13F are photographs of the corresponding serial sections that were stained with the secondary antibody alone.

FIGS. 14A-14H show photographs of breast cancer tissues stained with MNE6 anti-MUC1* antibody at 10 ug/mL, then stained with a rabbit anti mouse secondary HRP antibody. FIGS. 14A-14D are breast cancer tissues from patient #300. FIGS. 14E-14H are breast cancer tissues from metastatic patient #291.

FIGS. 15A-15F show photographs of normal lung and lung cancer tissues stained with humanized MNE6-scFv-Fc biotinylated anti-MUC1* antibody at 2.5 ug/mL, then stained with a secondary streptavidin HRP antibody. FIG. 15A is a normal lung tissue.

FIG. 15B15C are lung cancer tissues from patients as denoted in the figure. FIGS. 15D-15F are photographs of the corresponding serial sections that were stained with the secondary antibody alone.

FIGS. 16A-16F show photographs of normal lung and lung cancer tissues stained with humanized MNE6-scFv-Fc biotinylated anti-MUC1* antibody at 2.5 ug/mL, then stained with a secondary streptavidin HRP antibody. FIG. 16A is a normal lung tissue.

FIG. 16B16C are lung cancer tissues from patients as denoted in the figure. FIGS. 16D-16F are photographs of the corresponding serial sections that were stained with the secondary antibody alone.

FIGS. 17A-17F show photographs of normal lung and lung cancer tissues stained with humanized MNE6-scFv-Fc biotinylated anti-MUC1* antibody at 25 ug/mL, then stained with a secondary streptavidin HRP antibody. FIG. 17A is a normal lung tissue. FIGS. 17B-17C are lung cancer tissues from patients as denoted in the figure. FIGS. 17D-17F are photographs of the corresponding serial sections that were stained with the secondary antibody alone.

FIGS. 18A-18F show photographs of normal lung and lung cancer tissues stained with humanized MNE6-scFv-Fc biotinylated anti-MUC1* antibody at 25 ug/mL, then stained with a secondary streptavidin HRP antibody. FIG. 18A is a normal lung tissue. FIGS. 18B-18C are lung cancer tissues from patients as denoted in the figure. FIGS. 18D-18F are photographs of the corresponding serial sections that were stained with the secondary antibody alone.

FIGS. 19A-19D show photographs of normal small intestine and cancerous small intestine tissues stained with humanized MNE6-scFv-Fc biotinylated anti-MUC1* antibody at 5 ug/mL, then stained with a secondary streptavidin HRP antibody. FIG. 19A is a normal small intestine tissue. FIG. 19B is small intestine cancer from patient as denoted in the figure. FIGS. 19C-19D are photographs of the corresponding serial sections that were stained with the secondary antibody alone.

FIGS. 20A-20H show photographs of normal small intestine tissues stained with humanized MNE6-scFv-Fc anti-MUC1* antibody at 50 ug/mL, then stained with a secondary goat-anti-human HRP antibody. FIGS. 20A-20D are normal small intestine tissue. FIGS. 20E-20H are photographs of the corresponding serial sections that were stained with the secondary antibody alone.

FIGS. 21A-21H show photographs of cancerous small intestine tissues stained with humanized MNE6-scFv-Fc anti-MUC1* antibody at 50 ug/mL, then stained with a secondary goat-anti-human HRP antibody. FIGS. 21A-21D are cancerous small intestine tissue from a patient as denoted in figure. FIGS. 21E-21H are photographs of the corresponding serial sections that were stained with the secondary antibody alone.

FIGS. 22A-22H show photographs of cancerous small intestine tissues stained with humanized MNE6-scFv-Fc anti-MUC1* antibody at 50 ug/mL, then stained with a secondary goat-anti-human HRP antibody. FIGS. 22A-22D are cancerous small intestine tissue from a patient as denoted in figure. FIGS. 22E-22H are photographs of the corresponding serial sections that were stained with the secondary antibody alone.

FIGS. 23A-23H show photographs of normal colon tissues stained with humanized MNE6-scFv-Fc anti-MUC1* antibody at 50 ug/mL, then stained with a secondary goat-anti-human HRP antibody. FIGS. 23A-23D are normal colon. FIGS. 23E-23H are photographs of the corresponding serial sections that were stained with the secondary antibody alone.

FIGS. 24A-24H show photographs of colon cancer tissues stained with humanized MNE6-scFv-Fc anti-MUC1* antibody at 50 ug/mL, then stained with a secondary goat-anti-human HRP antibody. FIGS. 24A-24D are colon cancer tissue from a metastatic patient as denoted in figure. FIGS. 24E-24H are photographs of the corresponding serial sections that were stained with the secondary antibody alone.

FIGS. 25A-25H show photographs of colon cancer tissues stained with humanized MNE6-scFv-Fc anti-MUC1* antibody at 50 ug/mL, then stained with a secondary goat-anti-human HRP antibody. FIGS. 25A-25D are colon cancer tissue from a Grade 2 patient as denoted in figure. FIGS. 25E-25H are photographs of the corresponding serial sections that were stained with the secondary antibody alone.

FIGS. 26A-26H show photographs of colon cancer tissues stained with humanized MNE6-scFv-Fc anti-MUC1* antibody at 50 ug/mL, then stained with a secondary goat-anti-human HRP antibody. FIGS. 26A-26D are colon cancer tissue from a metastatic patient as denoted in figure. FIGS. 26E-26H are photographs of the corresponding serial sections that were stained with the secondary antibody alone.

FIGS. 27A-27H show photographs of prostate cancer tissues stained with humanized MNE6-scFv-Fc anti-MUC1* antibody at 50 ug/mL, then stained with a secondary goat-anti-human HRP antibody. FIGS. 27A-27D are prostate cancer tissue from a patient as denoted in figure. FIGS. 27E-27H are photographs of the corresponding serial sections that were stained with the secondary antibody alone.

FIGS. 28A-28H show photographs of prostate cancer tissues stained with humanized MNE6-scFv-Fc anti-MUC1* antibody at 50 ug/mL, then stained with a secondary goat-anti-human HRP antibody. FIGS. 28A-28D are prostate cancer tissue from a patient as denoted in figure. FIGS. 28E-28H are photographs of the corresponding serial sections that were stained with the secondary antibody alone.

FIGS. 29A-29H show photographs of prostate cancer tissues stained with humanized MNE6-scFv-Fc anti-MUC1* antibody at 50 ug/mL, then stained with a secondary goat-anti-human HRP antibody. FIGS. 29A-29D are prostate cancer tissue from a patient as denoted in figure. FIGS. 29E-29H are photographs of the corresponding serial sections that were stained with the secondary antibody alone.

FIGS. 30A-30F show photographs of a triple negative breast cancer array stained with anti-MUC1* antibody huMNC2scFv. The first score shown is the Allred score and the second is the tumor grade. The percentage of the array that scored zero, weak, medium or strong is graphed as a pie chart. FIG. 30A shows the pie chart of score of anti-MUC1* antibody staining. FIG. 30B shows a photograph of the array stained with the antibody. FIGS. 30C-30D show magnified photographs of two of the breast cancer specimens from the array. FIGS. 30E-30F show more magnified photographs of the portion of the specimen that is marked by a box.

FIGS. 31A-31F show photographs of an ovarian cancer array stained with anti-MUC1* antibody huMNC2scFv. The first score shown is the Allred score and the second is the tumor grade. The percentage of the array that scored zero, weak, medium or strong is graphed as a pie chart. FIG. 31A shows the pie chart of score of anti-MUC1* antibody staining. FIG. 31B shows a photograph of the array stained with the antibody. FIGS. 31C-31D show magnified photographs of two of the breast cancer specimens from the array. FIGS. 31E-31F show more magnified photographs of the portion of the specimen that is marked by a box.

FIGS. 32A-32F show photographs of a pancreatic cancer array stained with anti-MUC1* antibody huMNC2scFv. The first score shown is the Allred score and the second is the tumor grade. The percentage of the array that scored zero, weak, medium or strong is graphed as a pie chart. FIG. 32A shows the pie chart of score of anti-MUC1* antibody staining. FIG. 32B shows a photograph of the array stained with the antibody. FIGS. 32C-32D show magnified photographs of two of the breast cancer specimens from the array. FIGS. 32E-32F show more magnified photographs of the portion of the specimen that is marked by a box.

FIGS. 33A-33F show photographs of a lung cancer array stained with anti-MUC1* antibody huMNC2scFv. The first score shown is the Allred score and the second is the tumor grade. The percentage of the array that scored zero, weak, medium or strong is graphed as a pie chart. FIG. 33A shows the pie chart of score of anti-MUC1* antibody staining. FIG. 33B shows a photograph of the array stained with the antibody. FIGS. 33C-33D show magnified photographs of two of the breast cancer specimens from the array. FIGS. 33E-33F show more magnified photographs of the portion of the specimen that is marked by a box.

FIGS. 34A-34I show photographs of normal tissues stained with anti-MUC1* antibody huMNC2scFv.

FIGS. 35A-35D show FACS scans of cells expressing either no MUC1, MUC1* or full-length MUC1, wherein the cells were probed with either MNC2 or VU4H5. FIG. 35A shows MUC1 negative HCT-116 colon cancer cells probed with antibody MNC2. FIG. 35B shows HCT cells that have been transfected with MUC1* wherein the extra cellular domain is just the sequence of the PSMGFR peptide wherein the cells are probed with antibody MNC2. FIG. 35C shows HCT-MUC1-18 cells which are a cleavage resistant single cell clone of HCT cells transfected with full-length MUC1, also referred to herein as HCT-MUC1-41TR, and cells were probed with antibody MNC2. FIG. 35D shows HCT-MUC1-18 cells probed with antibody VU4H5 which is an antibody that recognizes the hundreds of tandem repeats epitopes in full-length MUC1. As can be seen in the figures, MNC2 recognizes an ectopic epitope that is not accessible in full-length MUC1.

FIGS. 36A-36D show Western blots and corresponding FACs analysis of HCT-116 cells which are a MUC1 negative colon cancer cell line, that were then stably transfected with either MUC1* or MUC1 full-length. The single cell clones that are shown are HCT-MUC1-41TR, and HCT-MUC1*. FIG. 36A shows a Western blot of the parent cell line HCT-116, HCT-MUC1-41TR and HCT-MUC1* wherein the gel has been probed with a rabbit polyclonal antibody, SDIX, that only recognizes cleaved MUC1. A visible band between 25 and 35 kDa can be readily seen in Lane 6, loaded with HCT-MUC1*, whereas there is only a faint band in Lanes 4 and 5, showing that only a small amount of MUC1 is cleaved in the HCT-MUC1-41Tr cells. There is no cleaved MUC1 present in the parent cell line HCT-116 loaded into Lanes 2 and 3. FIG. 36B is a Western blot that was probed with a mouse monoclonal antibody VU4H5 that recognizes the tandem repeats of full-length MUC1. As can be seen, only HCT-MUC1-41TR contains full-length MUC1. FIG. 36C shows FACS scans showing that HCT-MUC1* is 95.7% positive for SDIX which only binds to MUC1* and essentially not at all for MUC1 full-length. FIG. 36D shows FACS scans that show that HCT-MUC1-41TR cells are 95% positive for full-length MUC1 and only about 11% positive for the cleaved form, MUC1*.

FIG. 37A-37C shows western blots and a bar graph of FACS analysis assessing the ability of MNC2 to recognize a full-length MUC1 after it has been cleaved by MMP9. FIG. 37A shows a Western blot of HCT-MUC1-18 cells, which are a cleavage resistant cell line, to which was added cleavage enzyme MMP9. The cell lysate fraction was run on a gel and probed with a polyclonal anti-PSMGFR antibody. The photo shows that in a dose dependent manner, MMP9 cleaved MUC1 to MUC1*, the −25 kDa species. FIG. 37B shows the Western blot of the conditioned media from the same experiment. The photo shows that the addition of cleavage enzyme MMP9, in a dose dependent manner, increased the release of the tandem repeat domain into the conditioned media. FIG. 37C shows FACS analysis of the experiment. The graphs show that the addition of MMP9, in a dose dependent manner, increased recognition of the cleavage product by anti-MUC1* antibody MNC2 and decreased the recognition of the full-length MUC1 which contains the tandem repeat domain.

FIG. 38 shows a photograph of a Western blot in which HCT-MUC1-18 cells, labeled here as HCT-18, a cleavage resistant single cell clone of HCT cells transfected with full-length MUC1, are treated with varying amounts of a catalytically active ADAM17 or MMP14. Shed MUC1 tandem repeat domain of full-length MUC1 is immunoprecipitated from the conditioned media, and run on a gel that is then probed with VU4H5 that binds to the tandem repeat epitopes. As can be seen, MMP14 also efficiently cleaves MUC1 full-length and sheds the tandem repeat containing extra cellular domain into the conditioned media. Cleavage enzyme ADAM17 did not cleave MUC1.

FIG. 39A-39B shows fluorescence activated cell sorting (FACS) measurements of human CD34+ hematopoietic stem cells of human bone marrow stained with anti-MUC1* monoclonal antibodies MNC3, MNC2, MNE6 or an isotype control antibody. The histogram of the FACS assay and the bar graph showing the data show that the MUC1* positive cells of the bone marrow are recognized by one anti-MUC1* antibody, MNC3 but not by MNE6 or MNC2. All three antibodies bind to the PSMGFR peptide. The great difference in the specificity of these antibodies suggests that MNC3 recognizes a MUC1*-like form created when MUC1 is cleaved by an enzyme that is different from MMP9.

FIG. 40A-40G shows the details of FACS analysis of the hematopoietic stem cells probed with either MNC3 or MNE6. FIG. 40A shows the FACS scatter plot of total bone marrow cells. FIG. 40B shows the FACS scatter plot of the CD34+ cells. FIG. 40C shows the FACS histogram of the CD34+ cells. FIG. 40D shows the FACS scatter plot of the earliest hematopoietic stem cells, which are CD34+/CD38−, stained with either MNC3 or MNE6. FIG. 40E shows the histogram of the experiment. FIG. 40F shows the histogram overlay of MNC3 binding to CD34+/CD38−cells versus MNE6. FIG. 40G shows the bar graph of that FACS experiment.

FIG. 41A-42H shows the details of FACS analysis of CD34+/CD38^{-/lo} hematopoietic stem cells probed with a polyclonal anti-PSMGFR antibody SDIX, MNE6 or MNC2. FIG. 41A shows the FACS scatter plot of the CD34+/CD38^{-/lo} population of cells. FIG. 41E shows a table of the detailed analysis. FIG. 41B shows the FACS scatter plot of the CD34+/CD38^{-/lo} population of cells probed with the anti-PSMGFR polyclonal antibody SDIX. FIG. 41F shows a table of the detailed analysis. FIG. 41C shows the FACS scatter plot of the CD34+/CD38^{-/lo} population of cells probed with MNE6. FIG. 41G shows a table of the detailed analysis. FIG. 41D shows the FACS scatter plot of the CD34+/CD38^{-/lo} population of cells probed with MNC2. FIG. 41H shows a table of the detailed analysis.

FIG. 42A-42H shows photographs of DU145 prostate cancer cells or T47D breast cancer cells that have been treated with either the Fab of anti-MUC1* antibody MNC2, MNE6, MNC3 or MNC8. The images show that cancer specific antibodies MNC2 and MNE6 effectively kill prostate and breast cancer cells while the monoclonal antibodies MNC3 and MNC8 do not.

FIG. 43 shows a graph of a PCR experiment comparing expression of a wide range of cleavage enzymes expressed in different cells lines, wherein the values have been normalized to those expressed in breast cancer cell line T47D. Cell lines that are compared are prostate cancer cell line DU145, HCT-MUC1-41TR that is a MUC1 negative colon cancer cell line transfected with a MUC1 whose extracellular domain is truncated after 41 tandem repeat units and that is not cleaved to the MUC1* form, T47D breast cancer cell line and CD34+ bone marrow cells.

FIG. 43 shows a graph of a PCR experiment in which the expression levels of various cleavage enzymes are measured in DU145 prostate cancer cells, HCT116+ MUC1FL, also known as HCT-MUC1-18 a cell line expressing full-length MUC1, T47D breast cancer cells, and CD34+ hematopoietic stem cells of the bone marrow. The fold expression is relative to the expression of each cleavage enzyme in T47D breast cancer cells, set as 1.

FIG. 44 shows the graph of the PCR experiment of FIG. 43 but with the Y-axis maximum set to 5.

FIGS. 45A-45P show photographs of a CAR T co-culture assay in which the targeting antibody fragment of the CAR is huMNC2scFv wherein CAR44 has a CD8 transmembrane domain, followed by 41BB-3zeta and CAR50 has a CD4 transmembrane domain, followed by 41BB-3zeta. The target cancer cells are: HCT-FLR which is HCT-116 cells transfected with MUC1*45 and HCT-MUC1-41TR, which is a stable single cell clone HCT-116 cell line that expresses MUC1 with an extracellular domain truncated after 41 tandem repeats and that does not get cleaved to the MUC1* form on its own. The HCT-MUC1-41TR cancer cells were also incubated with conditioned media from cells transfected with MMP9 or ADAM17 before co-culture with the CAR T cells. Conditioned media of the MMP9 or ADAM17 expressing cells were also incubated with APMA which is an activator of those cleavage enzymes. The images shown are an overlay of the 4× bright field image and the fluorescent image of the same showing cancer cells dyed with a red CMTMR lipophilic dye. FIGS. 45A, 45E, 45I, 45M show photographs of cells co-cultured with untransduced human T cells. FIGS. 45B, 45F, 45J, 45N show photographs of cells co-cultured with human T cells transduced with anti-MUC1* CAR44 at an MOI of 10. FIGS. 45C, 45G, 45K, 450 show photographs of cells co-cultured with human T cells transduced with anti-MUC1* CAR50 at an MOI of 10. FIGS. 45D, 45H, 45L, 45P show photographs of cells co-cultured with human T cells transduced with anti-MUC1* CAR44 at an MOI of 50, which increases transduction efficiency. FIGS. 45B, 45C, 45D show that both CAR44 and CAR50 transduced T cells recognized MUC1* expressed in these cancer cells, bound to them, induced clustering and killed many cancer cells. FIGS. 45F, 45G, 45H show that neither CAR44 nor CAR50 transduced T cells recognize full-length MUC1 expressed in HCT-MUC1-41TR cancer cells. There is no T cell induced clustering and the number of cancer cells has not decreased. FIGS. 45J, 45K, 45L show that activated MMP9 has cleaved full-length MUC1 to a MUC1* form that is recognized by both CAR44 and CAR50 transduced T cells. There is clearly visible CAR T cell induced clustering and a decrease in the number of cancer cells as they are killed. FIGS. 45N, 450, 45P show that activated ADAM17 has either not cleaved MUC1 or cleaved it at a position not recognized by MNC2. Neither huMNC2-CAR44 nor huMNC2-CAR50 transduced T cells recognized these cancer cells.

FIG. 46A-46T shows photographs of a CAR T co-culture assay in which the targeting antibody fragment of the CAR is MNC2 scFv wherein CAR44 has a CD8 transmembrane domain, followed by 41BB-3zeta and CAR50 has a CD4 transmembrane domain, followed by 41BB-3zeta. The target cancer cells are breast cancer T47D cells that were also incubated with conditioned media from cells transfected with MMP2, MMP9 or ADAM17 before co-culture with the MNC2-CAR T cells. In some cases, the conditioned media of the MMP2 and MMP9 expressing cells were also incubated with APMA, which is an activator of these cleavage enzymes. The images shown are an overlay of the 4× bright field image and the fluorescent image of the same showing cancer cells dyed with a red CMTMR lipophilic dye. As can be seen, the MNC2-CAR T cells only bind to and attack the target cancer cells that express the cleaved form, MUC1*.

FIGS. 47A-47I show photographs of cancer cells co-cultured with anti-MUC1* CAR T cells, wherein some of the cancer cells were pre-incubated with activated MMP9 prior to co-culture with the CAR T cells. The cancer cells shown in FIGS. 47A-47C are MUC1 negative colon cancer cell line HCT-116 that have been stably transfected to express MUC1*. The cancer cells shown in FIGS. 47D-47F are MUC1 positive breast cancer cell line T47Ds that express high levels of both MUC1 full-length and MUC1*. The cancer cells shown in FIGS. 47G-47I are MUC1 positive breast cancer cell line T47Ds that were pre-incubated with activated MMP9. The cells shown in FIGS. 47A, 47D and 47G were co-cultured with untransduced human T cells and are the controls. The cells shown in FIGS. 47B, 47E and 47H were co-cultured with human T cells that were transduced with huMNC2-CAR44 at an MOI of 10, wherein MOI stands for multiplicity of infection and the higher the MOI the more CARs are expressed on the T cells. The cells shown in FIGS. 47C, 47F and 47I were co-cultured with human T cells that were transduced with huMNC2-CAR44 at an MOI of 50. As can be seen in the photographs, the CAR44 T cells bind to the target MUC1* positive cancer cells, surrounding and killing them. Comparing the photograph FIG. 47I with the others, it can be seen that the cells that were pre-incubated with MMP9 become much more susceptible to CAR T killing when the antibody targeting head of the CAR recognizes MUC1*. It also demonstrates that MUC1 cleaved by MMP9 is recognized by huMNC2scFv.

FIG. 48 shows an xCelligence graph of T47D breast cancer cells in co-culture with either untransduced T cells, as a control, or huMNC2-CAR44 T cells over a 45 hour period. After 18 hours of cancer cell growth, a catalytic sub-unit MMP9 was added to some of the cells. At 25 hours, T cells were added. As can be seen, huMNC2-CAR44 T cell killing is greatly improved when the T47D cells are pre-incubated with cleavage enzyme MMP9. In the xCelligence system, target cancer cells, which are adherent, are plated onto electrode array plates. Adherent cells insulate the electrode and increase the impedance. The number of adherent cancer cells is directly proportional to impedance. T cells are not adherent and do not contribute to impedance. Therefore, increasing impedance reflects growth of cancer cells and decreasing impedance reflects killing of cancer cells.

FIG. 49 shows an xCelligence graph of DU145 prostate cancer cells in co-culture with either untransduced T cells, as a control, or huMNC2-CAR44 T cells over a 45 hour period. After 18 hours of cancer cell growth, a catalytic sub-unit MMP9 was added to some of the cells. At 25 hours, T cells were added. As can be seen, huMNC2-CAR44 T cell killing is not affected by pre-incubation with cleavage enzyme MMP9. DU145 cancer cells express a significantly lower amount of MUC1 which includes the full-length form as well as MUC1*. The lower density of MUC1 full-length does not sterically hinder T cell access to the membrane proximal MUC1*.

FIG. 50 shows a bar graph of a PCR experiment measuring the amount of MUC1 expressed by a panel of cell lines and primary cells, comprised of normal cells as well as cancer cells.

FIG. 51A-51B shows a bar graph of an ELISA assay measuring the amount of interferon gamma, IFN-g, secreted by huMNC2-CAR44 human T cells after co-culture with the normal cells or the HCT-MUC1* cancer cells for 72 hours. FIG. 51A shows the results of the experiment where the CAR44 T cell to target cell ratio was 1:1. FIG. 51B shows the results of the experiment where the CAR44 T cell to target cell ratio was 0.5:1.

FIG. 52A-52B shows a bar graph of an ELISA assay measuring the amount of interleukin-2, IL-2, secreted by huMNC2-CAR44 human T cells after co-culture with the normal cells or the HCT-MUC1* cancer cells for 72 hours. FIG. 52A shows the results of the experiment where the CAR44 T cell to target cell ratio was 1:1. FIG. 52B shows the results of the experiment where the CAR44 T cell to target cell ratio was 0.5:1.

FIG. 53A-53J shows bar graphs of FACS analysis of live versus dead markers and photographs of normal cells versus cancer cells after co-culture with huMNC2-CAR44 T cells. FIG. 53A.1 shows the bar graph of FACS analysis of live versus dead cells after HCT-MUC1* cancer cells were co-cultured with huMNC2-CAR44 T cells. FIG. 53A.2 and FIG. 53A.3 show the photographs of the experiment described in FIG. 53A.1. FIG. 53B.1 shows the bar graph of FACS analysis of live versus dead cells after MCF-12A normal breast cells were co-cultured with huMNC2-CAR44 T cells. FIG. 53B.2 and FIG. 53B.3 show the photographs of the experiment described in FIG. 53B.1. FIG. 53C.1 shows the bar graph of FACS analysis of live versus dead cells after THLE-3 normal liver cells were co-cultured with huMNC2-CAR44 T cells. FIG. 53C.2 and FIG. 53C.3 show the photographs of the experiment described in FIG. 53C.1. FIG. 53D.1 shows the bar graph of FACS analysis of live versus dead cells after T/G HA-HSMC normal heart cells were co-cultured with huMNC2-CAR44 T cells. FIG. 53D.2 and FIG. 53D.3 show the photographs of the experiment described in FIG. 53D.1. FIG. 53E.1 shows the bar graph of FACS analysis of live versus dead cells after Hs1.Tes normal testes cells were co-cultured with huMNC2-CAR44 T cells. FIG. 53E.2 and FIG. 53E.3 show the photographs of the experiment described in FIG. 53E.1. FIG. 53F.1 shows the bar graph of FACS analysis of live versus dead cells after HEK-293 MUC1 negative cells were co-cultured with huMNC2-CAR44 T cells. FIG. 53F.2 and FIG. 53F.3 show the photographs of the experiment described in FIG. 53F.1. FIG. 53G.1 shows the bar graph of FACS analysis of live versus dead cells after HRCE normal kidney cells were co-cultured with huMNC2-CAR44 T cells. FIG. 53G.2 and FIG. 53G.3 show the photographs of the experiment described in FIG. 53G.1. FIG. 53H.1 shows the bar graph of FACS analysis of live versus dead cells after CCD-18Lu normal lung cells were co-cultured with huMNC2-CAR44 T cells. FIG. 53H.2 and FIG. 53H.3 show the photographs of the experiment described in FIG. 53H.1. FIG. 53I.1 shows the bar graph of FACS analysis of live versus dead cells after HBEC-5i normal brain cells were co-cultured with huMNC2-CAR44 T cells. FIG. 53I.2 and FIG. 53I.3 show the photographs of the experiment described in FIG. 53I.1. FIG. 53J.1 shows the bar graph of FACS analysis of live versus dead cells after Hs.738.St/Int normal stomach and intestine cells were co-cultured with huMNC2-CAR44 T cells. FIG. 53J.2 and FIG. 53J.3 show the photographs of the experiment described in FIG. 53J.1.

FIG. 54 shows photographs of a breast cancer tissue array in which for each patient there is a specimen from the primary tumor plus a specimen from that patient's metastasis. As can be seen in the figure, most often the metastasis expresses more MUC1* than the primary tumor.

FIGS. 55A-55H show the cytotoxic effect of huMNC2-CAR44 T cells on MUC1* positive DU145 prostate cancer cells as measured by a variety of assays. FIG. 55A is a fluorescent photograph of untransduced T cells co-cultured with the prostate cancer cells, wherein granzyme B is stained with a red fluorophore. FIG. 55B shows merging of DAPI and granzyme B. FIG. 55C is a fluorescent photograph of huMNC2-CAR44 T cells co-cultured with the prostate cancer cells, wherein granzyme B is stained with a red fluorophore. FIG. 55D shows merging of DAPI and granzyme B. FIG. 55E is a FACS scan for fluorescently labeled granzyme B for untransduced T cells incubated with the cancer cells. FIG. 55F is a FACS scan showing a positive increase in fluorescently labeled granzyme B for huMNC2-CAR44 T cells incubated with the cancer cells. FIG. 55G is a graph of the mean fluorescent intensity. FIG. 55H is an xCELLigence scan tracking the real-time killing of DU145 cancer cells by huMNC2-CAR44 T cells (blue trace) but not by untransduced T cells (green).

FIGS. 56A-56H show the cytotoxic effect of huMNC2-CAR44 T cells on MUC1* positive CAPAN-2 pancreatic cancer cells as measured by a variety of assays. FIG. 56A is a fluorescent photograph of untransduced T cells co-cultured with the pancreatic cancer cells, wherein granzyme B is stained with a red fluorophore. FIG. 56B shows merging of DAPI and granzyme B. FIG. 56C is a fluorescent photograph of huMNC2-CAR44 T cells co-cultured with the pancreatic cancer cells, wherein granzyme B is stained with a red fluorophore. FIG. 56D shows merging of DAPI and granzyme B. FIG. 56E is a FACS scan for fluorescently labeled granzyme B for untransduced T cells incubated with the cancer cells. FIG. 56F is a FACS scan showing a positive increase in fluorescently labeled granzyme B for huMNC2-CAR44 T cells incubated with the cancer cells. FIG. 56G is a graph of the mean fluorescent intensity. FIG. 56H is an xCELLigence scan tracking the real-time killing of CAPAN-2 cancer cells by huMNC2-CAR44 T cells (blue trace) but not by untransduced T cells (green).

FIGS. 57A-57C show xCELLigence scans tracking the real-time killing of MUC1* positive cancer cells, but not MUC1* negative cells, by huMNC2-CAR44 T cells. FIG. 57A shows that huMNC2-CAR44 T cells effectively kill HCT colon cancer cells that have been stably transfected with MUC1*. FIG. 57B shows that huMNC2-CAR44 T cells have almost no effect on HCT-MUC1-41TR, which is a MUC1 negative cancer cell that has been stably transfected with a MUC1 full-length. In this cell line only about 10% of the cells have MUC1 cleaved to MUC1*. FIG. 57C shows that huMNC2-CAR44 T cells have no effect on HCT-116 cells, which is a MUC1 negative colon cancer cell line.

FIG. 58A-58F shows photographs NOD/SCID/GAMMA mice in an IVIS instrument measuring photon emission from tumor cells after mice were treated with nothing, PBS, untransduced human T cells or huMNC2-CAR44 T cells. Mice had been injected sub-cutaneously with HCT-MUC1* tumor cells that had been made Luciferase positive. Ten (10) minutes before the IVIS photographs were taken, the mice were injected into the intraperitoneal (ip) space with the Luciferase substrate, Luciferin. FIG. 58A shows the tumor bearing mice that had only been treated with phosphate buffered saline, PBS. FIG. 58B shows the tumor bearing mice that had only been treated with untransduced T cells. FIG. 58C shows the tumor bearing mice that had been treated with a single dose of huMNC2-CAR44 T cells. FIG. 58D shows color scale of the images. FIG. 58E shows Kaplan-Meier survival curves of the experiment. FIG. 58F shows a table detailing the molecular makeup of the human T cells that were isolated from the mouse blood after sacrifice.

FIG. 59A-59C shows photographs NOD/SCID/GAMMA mice in an IVIS instrument measuring photon emission from tumor cells after mice were treated with nothing, PBS or huMNC2-CAR44 T cells. Mice had been injected sub-cutaneously with T47D-wt breast cancer cells or T47D+ more MUC1*, which is a mixed population of cells wherein 95% of the cells were T47D cells that had been stably transfected with even more MUC1*. Both T47D-wt and T47D plus more MUC1* cells had been made Luciferase positive. Ten (10) minutes before the IVIS photographs were taken, the mice were injected into the intraperitoneal (ip) space with the Luciferase substrate, Luciferin. FIG. 59A shows the tumor bearing mice that had only been treated with phosphate buffered saline, PBS. FIG. 59B shows the T47D-wt tumor bearing mice that had been treated with two (2) doses of huMNC2-CAR44 T cells. FIG. T90.1C shows the T47D-MUC1* tumor bearing mice that had been treated with two (2) doses of huMNC2-CAR44 T cells.

FIG. 60A-60C shows photographs NOD/SCID/GAMMA mice in an IVIS instrument measuring photon emission from tumor cells after mice were treated with nothing, PBS, untransduced T cells or huMNC2-CAR44 T cells. Mice had been injected sub-cutaneously with a mixed population of 70% T47D-wt breast cancer cells and 30% T47D cells that had been transfected with even more MUC1*. Both cell types had been made Luciferase positive. Ten (10) minutes before the IVIS photographs were taken, the mice were injected into the intraperitoneal (ip) space with the Luciferase substrate, Luciferin. FIG. 60A shows the tumor bearing mice that had only been treated with phosphate buffered saline, PBS. FIG. 60B shows tumor bearing mice that had only been treated with untransduced T cells. FIG. 60C shows the tumor bearing mice that had been treated with two (2) doses of huMNC2-CAR44 T cells.

FIGS. 61A-61J show fluorescent photographs of mice taken on an IVIS instrument. NSG (NOD/SCID/GAMMA) immune compromised mice that on Day 0 were sub-cutaneously injected into the flank with 500K human BT-20 cells which are a MUC1* positive triple negative breast cancer cell line. The cancer cells had been stably transfected with Luciferase. Tumors were allowed to engraft. On Day 6 after IVIS measurement, animals were given a one-time injection of 10 million of either human T cells transduced with huMNC2-scFv-CAR44 or untransduced T cells. 5 million T cells were injected intra-tumor and 5 million were injected into the tail vein. 10 minutes prior to IVIS photographs, mice were IP injected with Luciferin, which fluoresces after cleavage by Luciferase, thus making tumor cells fluoresce. FIGS. 61A, 61D, 61G show photographs of mice that were treated with huMNC2-scFv-CAR44 T cells that had been pre-stimulated by co-culturing for 24 hours with 4 μm beads to which was attached a synthetic MUC1*, PSMGFR peptide 24 hours prior to administration: Protocol 1. FIGS. 61B, 61E, 61H show photographs of mice that were treated with huMNC2-scFv-CAR44 T cells that had been pre-stimulated by twice co-culturing for 24 hours with MUC1* positive cancer cells 24 hours prior to administration: Protocol 2. FIGS. 61C, 61F, 61I show photographs of mice that were treated with untransduced human T cells. FIG. 61J is a color scale relating fluorescence in photons/second to color.

FIGS. 62A-62M show fluorescent photographs of mice taken on an IVIS instrument. NSG (NOD/SCID/GAMMA) immune compromised mice that on Day 0 were injected into the intraperitoneal cavity (IP) with 500K human SKOV-3 cells which are a MUC1* positive ovarian cancer cell line. The cancer cells had been stably transfected with Luciferase. Tumors were allowed to engraft. On Day 4, animals were injected into the intraperitoneal space with 10M either human T cells transduced with huMNC2-scFv-CAR44, untransduced T cells or PBS. On Day 11, animals were injected again except that half the cells were injected into the tail vein and the other half was IP injected. Animals were imaged by IVIS on Days 3, 7, 10 and 15. 10 minutes prior to IVIS photographs, mice were IP injected with Luciferin, which fluoresces after cleavage by Luciferase, thus making tumor cells fluoresce. FIGS. 62A, 62D, 62G, and 62J show photographs of mice that were treated with huMNC2-scFv-CAR44 T cells that had been pre-stimulated by co-culturing for 24 hours with 1 μm beads to which was attached a synthetic MUC1*, PSMGFR peptide 24 hours prior to administration. FIGS. 62B, 62E, 62H, and 62K show photographs of mice that were treated with untransduced human T cells. FIGS. 62C, 62F, 62I, and 62L show photographs of mice that were treated with PBS. FIGS. 62A, 62B and 62C are IVIS images taken Day 3 prior to CAR T, T cell or PBS administration. FIGS. 62D, 62E and 62F show IVIS images of animals on Day 7, just four (4) days after treatment. FIGS. 62G, 62H, and 62I show IVIS images of animals on Day 10. FIGS. 62J, 62K, and 62L show IVIS images of animals on Day 15 FIG. 62M is the IVIS color scale relating fluorescence in photons/second to color.

FIG. 63 shows a graph of an ELISA binding assay in which various monoclonal antibodies are tested for their ability to bind to the PSMGFR peptide, the N-10, C-10, N+20/C-27, or the N+9/C-9 peptide, wherein the concentration of the antibody was at 10 ug/mL or 1 ug/mL. Note that anti-MUC1* monoclonal antibodies C2 and E6, which have been demonstrated to be cancer specific, bind to the PSMGFR peptide, still bind if the 10 N-terminal amino acids are missing, but do not bind if the 10 or 9 C-terminal amino acids are missing.

FIG. 64A-64B shows a graph of an ELISA binding assay. The antibodies being tested were derived from animals immunized with the PSMGFR peptide. The first selection criteria was to confirm that the antibodies bound to the immunizing PSMGFR peptide. FIG. 64A shows a graph of an ELISA of selected antibodies that were further tested to determine their ability to bind to the PSMGFR peptide, the N-10, the C-10, N+20/C-27, or N+9/C-9 peptide. All the antibodies except 18B4 were able to bind to the N-10 peptide. 18B4 recognized N+20/C-27 but not the N-10 peptide, implying that its cognate epitope lies within the GTINVHDVET sequence. All except 20A10 and C2 showed some binding to the C-10 and N+9/C-9 peptide, showing that both 20A10 and C2 require the 10 membrane proximal amino acids for binding. C2, which requires the 10 membrane proximal amino acids for binding has been demonstrated to be cancer specific. FIG. 64B shows the sequences of the various peptides. The color of the bars for each antibody in the ELISA graph are color coded to match the deductive cognate sequence, or a portion thereof, of that antibody.

FIG. 65A-65B shows a graph of an ELISA binding assay in which various monoclonal antibodies are tested for their ability to bind to the PSMGFR peptide, the N-10, the C-10, N+20/C-27, or N+9/C-9 peptide. The antibodies being tested were derived from animals immunized with the N+20/C-27 peptide. The first selection criteria was to confirm that the antibodies bound to the immunizing N+20/C-27 peptide. FIG. 65A shows a graph of ELISA binding assay that tests the ability of each antibody to bind to various peptides. Although these antibodies were raised against the N+20/C-27 peptide, all but one, 45C11, still bind to the PSMGFR peptide. The binding of 45C11 is weak but deductive reasoning shows that the cognate epitope must lie within the SNIKFRPGSVV sequence. 1E4 was able to bind to the N+20/C-27 peptide, the PSMGFR and the N-10 peptide, consistent with the idea that its epitope must lie within the QFNQYKTE sequence. FIG. 65B shows the sequences of the various peptides. The color of the bars for each antibody in the ELISA graph are color coded to match the deductive cognate sequence, or a portion thereof, of that antibody.

FIG. 66A-66B shows a graph of an ELISA binding assay in which various monoclonal antibodies are tested for their ability to bind to the PSMGFR peptide, the N-10, the C-10, N+20/C-27, or N+9/C-9 peptide. The antibodies being tested were derived from animals immunized with the N+9/C-9 peptide. The first selection criteria was to confirm that the antibodies bound to the immunizing N+9/C-9 peptide. FIG. 66A shows a graph of the ELISA assay. All but one, 39H5, were only able to bind to the immunizing peptide, N+9/C-9. 39H5 showed very weak binding to the PSMGFR and N-10 peptide, consistent with the idea that at least a portion of its cognate epitope must lie within the QFNQYKTE sequence. FIG. 66B shows the sequences of the various peptides. The color of the bars for each antibody in the ELISA graph are color coded to match the deductive cognate sequence, or a portion thereof, of that antibody.

FIG. 67A-67D shows results of ELISA assays to further define antibody epitopes within the MUC1 or MUC1* extra cellular domain. The antibodies shown in this figure were all generated by immunizing animals with the PSMGFR peptide. Binding assays tested antibodies for their ability to bind to peptides N-19, N-26, N-30, N-10/C-5, N-19/C-5, PSMGFR, N-10 and C-10, which are all subsets of the PSMGFR peptide and numbering refers back to the PSMGFR peptide. FIG. 67A shows the binding of the various antibodies to the various peptides. FIG. 67B shows the sequence of the PSMGFR peptide that has been extended 20 amino acids at the N-terminus. FIG. 67C shows the sequences of the PSMGFR-derived subset peptides. FIG. 67D shows the sequences that comprise all or part of the epitope that is essential for antibody recognition.

FIG. 68A-68D shows results of ELISA assays to further define antibody epitopes within the MUC1 or MUC1* extra cellular domain. The antibodies shown in this figure were all generated by immunizing animals with the N+20/C-27 peptide. Binding assays tested antibodies for their ability to bind to peptides N-19, N-26, N-30, N-10/C-5, N-19/C-5, PSMGFR, N-10 and C-10, which are all subsets of the PSMGFR peptide and numbering refers back to the PSMGFR peptide. FIG. 68A shows the binding of the various antibodies to the various peptides. FIG. 68B shows the sequence of the PSMGFR peptide that has been extended 20 amino acids at the N-terminus. FIG. 68C shows the sequences of the PSMGFR-derived subset peptides. FIG. 68D shows the sequences that comprise all or part of the epitope that is essential for antibody recognition.

FIGS. 69A-69D show results of ELISA assays to further define antibody epitopes within the MUC1 or MUC1* extra cellular domain. The antibodies shown in this figure were all generated by immunizing animals with the N+9/C-9 peptide. Binding assays tested antibodies for their ability to bind to peptides N-19, N-26, N-30, N-10/C-5, N-19/C-5, PSMGFR, N-10 and C-10, which are all subsets of the PSMGFR peptide and numbering refers back to the PSMGFR peptide. FIG. 69A shows the binding of the various antibodies to the various peptides. FIG. 69B shows the sequence of the PSMGFR peptide that has been extended 20 amino acids at the N-terminus. FIG. 69C shows the sequences of the PSMGFR-derived subset peptides. FIG. 69D shows the sequences that comprise all or part of the epitope that is essential for antibody recognition.

FIG. 70A-70B shows a graph of an ELISA displacement assay. In this experiment, a multi-well plate was coated with the PSMGFR peptide. In FIG. 70A, recombinant NME7A was allowed to bind to the surface-immobilized PSMGFR peptide. Various antibodies were added, followed by a wash step. The amount of NME7A that remained attached to the PSMGFR coated plate, after antibody competition, was measured by detecting a tag on the NME7A. As a control, anti-NME7A antibodies were also tested for their ability to displace NME7A from the PSMGFR. As can be seen in the graph, antibodies MNC2, MNE6, 20A10, 3C2B1 and 5C6F3 displace NME7A from binding to the PSMGFR peptide, which is an indicator of the antibody being cancer-specific. FIG. 70B shows that the epitope within the MUC1* extracellular domain to which these antibodies bind is the sequence SVSDVPFPFSAQSGA, wherein binding is destroyed for MNC2, MNE6, 20A10, 3C2B1 if amino acids FPFS are not present or mutated and binding is destroyed for 5C6F3 if amino acids SVSDV are not present or mutated.

FIG. 71A-71H shows photographs of Western blots in which antibodies are tested for their ability to bind to a linear epitope in full-length MUC1 or MUC1*. FIG. 71A-71D shows testing of antibodies for ability to bind to a MUC1 negative cell line, HCT-116, or engineered cell lines HCT-MUC1-18, which is a cleavage resistant clone that expresses full-length MUC1, or HCT-MUC1*, which is engineered to express only the PSMGFR sequence in its extra cellular domain. FIG. 71E-71H shows testing of antibodies for ability to bind to breast cancer cell lines T47D or 1500 aka ZR-75-1. FIG. 71A and FIG. 71E show MNC2, a monoclonal antibody raised against PSMGFR peptide that binds to N-10 but not C-10 variants of the PSMGFR peptide. FIG. 71B and FIG. 71F show MNE6, a monoclonal antibody raised against PSMGFR peptide that binds to N-10 but not C-10 variants of the PSMGFR peptide. FIG. 71C and FIG. 71G show SDIX, a polyclonal antibody raised against PSMGFR peptide and which binds to the PSMGFR peptide. FIG. 71D and FIG. 71H show VU4H5, a commercially available monoclonal antibody that binds to the tandem repeats of full-length MUC1. As can be seen, neither MNC2 nor MNE6 bind linear epitopes of MUC1 species.

FIG. 72A-72P shows photographs of Western blots in which antibodies are tested for their ability to bind to a linear epitope in full-length MUC1 or MUC1*. All these antibodies were raised against the PSMGFR peptide and bind to the PSMGFR peptide. FIG. 72A-72H shows testing of antibodies for ability to bind to a MUC1 negative cell line, HCT-116, or engineered cell lines HCT-MUC1-18, which is a cleavage resistant clone that expresses full-length MUC1, or HCT-MUC1*, which is engineered to express only the PSMGFR sequence in its extra cellular domain. FIG. 72I-72P shows testing of antibodies for ability to bind to breast cancer cell lines T47D or 1500 aka ZR-75-1. FIG. 72A and FIG. 72I show 20A10. FIG. 72B and FIG. 72J show 25E6. FIG. 72C and FIG. 72K show 18B4. FIG. 72D and FIG. 72L show 18G12. FIG. 72E and FIG. 72M show 28F9. FIG. 72F and FIG. 72N show 3C2B1. FIG. 72G and FIG. 72O show 5C6F3. FIG. 72H and FIG. 72P show 5C6F3 wherein the blot has been exposed for a longer time period to render more visible the MUC1* specific bands. As can be seen, antibodies 25E6, 18B4 and to a degree 5C6F3 recognize linear epitopes but 20A10, 3C2B1, 18G12 and 28F9 do not.

FIG. 73A-73J shows photographs of Western blots in which antibodies are tested for their ability to bind to a linear epitope in full-length MUC1 or MUC1*. All these antibodies were raised against the N+20/C-27 variant of the PSMGFR peptide and bind to the N+20/C-27 peptide. FIG. 73A-73E shows testing of antibodies for ability to bind to a MUC1 negative cell line, HCT-116, or engineered cell lines HCT-MUC1-18, which is a cleavage resistant clone that expresses full-length MUC1, or HCT-MUC1*, which is engineered to express only the PSMGFR sequence in its extra cellular domain. FIG. 73F-73J shows testing of antibodies for ability to bind to breast cancer cell lines T47D or 1500 aka ZR-75-1. FIG. 73A and FIG. 73F show 1E4. FIG. 73B and FIG. 73G show 45C11. FIG. 73C and FIG. 73H show 31A1. FIG. 73D and FIG. 73I show 32C1. FIG. 73E and FIG. 73J show 29H1. As can be seen, antibodies 31A1 and 32C1 recognize linear epitopes.

FIG. 74A-74H shows photographs of Western blots in which antibodies are tested for their ability to bind to a linear epitope in full-length MUC1 or MUC1*. All these antibodies were raised against the N+9/C-9 variant of the PSMGFR peptide and bind to the N+9/C-9 peptide. FIG. 74A-74D shows testing of antibodies for ability to bind to a MUC1 negative cell line, HCT-116, or engineered cell lines HCT-MUC1-18, which is a cleavage resistant clone that expresses full-length MUC1, or HCT-MUC1*, which is engineered to express only the PSMGFR sequence in its extra cellular domain. FIG. 74E-74H shows testing of antibodies for ability to bind to breast cancer cell lines T47D or 1500 aka ZR-75-1. FIG. 74A and FIG. 74E show 8A9. FIG. 74B and FIG. 74F show 17H6. FIG. 74C and FIG. 74G show 3C5. FIG. 74D and FIG. 74H show 39H5.

FIG. 75A-75P show graphs of FACS analysis. HCT-MUC1-18 cells, which express full-length MUC1, were incubated with a catalytically active MMP9 or MMP2 for 24 hours, incubated with an antibody of the invention and then analyzed by FACS to see if the antibody bound to the MMP9 or the MMP2 cleaved form of MUC1. Note that the first bar of each graph shows that none of the antibodies binds to full-length MUC1 in the absence of cleavage. Each bar graph is labeled with both the name of the antibody used in that assay and its cognate epitope. The order of the graphs from right to left corresponds to the distance the from the cell surface of the antibody's cognate epitope. FIG. 75A shows antibody 1E4. FIG. 75B shows antibody 28F9. FIG. 75C shows antibody 18G12. FIG. 75D shows antibody 25E6. FIG. 75E shows antibody 20A10. FIG. 75F shows antibody 3C5. FIG. 75G shows antibody 29H1. FIG. 75H shows antibody 32C1. FIG. 75I shows antibody 31A1. FIG. 75J shows antibody 18B4. FIG. 75K shows antibody 45C11. FIG. 75L shows antibody 8A9. FIG. 75M shows antibody 17H6. FIG. 75N shows antibody 39H5. FIG. 75O shows antibody 3C2B1. FIG. 75P shows antibody 5C6F3.

FIG. 76A-76J show graphs of FACS analyses of reference antibodies MNC2, “C2”, and VU4H5 binding to either the MUC1-negative cell line HCT-116, HCTs transfected with MUC1*, “HCT-MUC1*”, a cleavage resistant single cell clone of HCTs transfected with MUC1 full-length, “HCT-MUC1-18”, and MNC2 binding to breast cancer cells line T47D or breast cancer cell line 1500 also known as ZR-75-1. MNC2 binds to an ectopic binding site on the extra cellular domain of MUC1*, within the membrane proximal portion of the PSMGFR sequence. The MNC2 binding site is only available after cleavage and release of the bulk of the extra cellular domain comprising the tandem repeat domain. VU4H5 binds to hundreds of repeating epitopes in the tandem repeat domain. FIG. 76A-76E show percent binding and FIG. 76F-76J show Mean Fluorescent Intensity or MFI.

FIG. 77A-77N show graphs of FACS analyses of reference antibody MNC2, “C2”, binding to a panel of cancer cell lines that are MUC1* positive, with the exception of MDA-MB-231, which expresses MUC1 and MUC1* at a level that is so low that it is often used as a negative control. MNC2 binds to an ectopic binding site on the extra cellular domain of MUC1*, within the membrane proximal portion of the PSMGFR sequence. The MNC2 binding site is only available after cleavage and release of the bulk of the extra cellular domain comprising the tandem repeat domain. FIG. 77A-77G show percent binding and FIG. 77H-77N show Mean Fluorescent Intensity or MFI. FIGS. 77A and 77H show the antibodies binding to lung cancer cell line NCI-H292. FIGS. 77B and 77I show the antibodies binding to lung cancer cell line NCI-H1975. FIGS. 77C and 77J show the antibodies binding to ovarian cancer cell line SKOV-3. FIGS. 77D and 77K show the antibodies binding to pancreatic cancer cell line HPAF-II. FIGS. 77E and 77L show the antibodies binding to pancreatic cancer cell line Capan-1. FIGS. 77F and 77M show the antibodies binding to prostate cancer cell line DU145. FIGS. 77G and 77N show the antibodies binding to breast cancer cell line MDA-MB-231, which is nearly MUC1 and MUC1* negative.

FIG. 78A-78C shows a color coded schematic of the basic PSMGFR sequence that has been extended or deleted at both the N- and C-termini. Antibodies of the invention were tested against this subset of peptides to further refine the epitopes to which each antibody binds or the critical amino acids within the epitope to which each antibody binds. FIG. 78A is an aligned schematic of the various subsets of peptides. FIG. 78B lists the antibodies that bind to each of the color coded sequences. FIG. 78C lists the cancer cell lines that each antibody recognizes.

FIG. 79A-79I shows color coded graphs that resulted from FACS analyses of each antibody binding to T47D breast cancer cells and their respective cognate sequences within the N-terminally extended PSMGFR sequence. FIG. 79A-79D are FACS graphs showing the percent cells that were recognized by each antibody. FIG. 79E-79H are FACS graphs showing the Mean Fluorescence Intensity, MFI, of each antibody. FIG. 79A and FIG. 79E show the FACS graph of antibodies that were generated by immunizing with the PSMGFR peptide. FIG. 79B and FIG. 79F show the FACS graph of antibodies that were generated by immunizing with the N+20/C-27 peptide. FIG. 79C and FIG. 79G show the FACS graph of antibodies that were generated by immunizing with the N+9/C-9 peptide. FIG. 79D and FIG. 79H also show the FACS graph of antibodies that were generated by immunizing with the PSMGFR peptide. FIG. 79I shows the PSMGFR sequence that is extended at the N-terminus by 20 amino acids. FIG. 79A, FIG. 79E show that 20A10 recognizes MUC1* as it exists on T47D breast cancer cells. FIG. 79D, FIG. 79H show that 3C2B1 recognizes MUC1* as it exists on T47D breast cancer cells. FIG. 79D, FIG. 79H show that 5C6F3 recognizes MUC1* as it exists on T47D breast cancer cells.

FIG. 80A-80I shows color coded graphs that resulted from FACS analyses of each antibody binding to 1500, also known as ZR-75-1, breast cancer cells and their respective cognate sequences within the N-terminally extended PSMGFR sequence. FIG. 80A-80C are FACS graphs showing the percent cells that were recognized by each antibody. FIG. 80D-80F are FACS graphs showing the Mean Fluorescence Intensity, MFI, of each antibody. FIG. 80A, FIG. 80E FIG. 80D and FIG. 80H show the FACS graph of antibodies that were generated by immunizing with the PSMGFR peptide. FIG. 80B and FIG. 80F show the FACS graph of antibodies that were generated by immunizing with the N+20/C-27 peptide. FIG. 80C and FIG. 80G show the FACS graph of antibodies that were generated by immunizing with the N+9/C-9 peptide. FIG. 80I shows the PSMGFR sequence that is extended at the N-terminus by 20 amino acids. FIG. 80A, FIG. 80E show that antibody 20A10 recognizes MUC1* as it exists on 1500 aka ZR-75-1 breast cancer cells. FIG. 80D, FIG. 80H show that antibody 3C2B1 recognizes MUC1* as it exists on 1500 aka ZR-75-1 breast cancer cells. FIG. 80D, FIG. 80H show that antibody 5C6F3 recognizes MUC1* as it exists on 1500 aka ZR-75-1 breast cancer cells.

FIG. 81A-81G shows color coded graphs that resulted from FACS analyses of each antibody binding to NCI-H292 lung cancer cells and their respective cognate sequences within the N-terminally extended PSMGFR sequence. FIG. 81A-81C are FACS graphs showing the percent cells that were recognized by each antibody. FIG. 81D-81F are FACS graphs showing the Mean Fluorescence Intensity, MFI, of each antibody. FIG. 81A and FIG. 81D show the FACS graph of antibodies that were generated by immunizing with the PSMGFR peptide. FIG. 81B and FIG. 81E show the FACS graph of antibodies that were generated by immunizing with the N+20/C-27 peptide. FIG. 81C and FIG. 81F show the FACS graph of antibodies that were generated by immunizing with the N+9/C-9 peptide. FIG. 81G shows the PSMGFR sequence that is extended at the N-terminus by 20 amino acids. FIG. 81A, FIG. 81D show that antibody 20A10 recognizes MUC1* as it exists on H292 lung cancer cells.

FIG. 82A-82G shows color coded graphs that resulted from FACS analyses of each antibody binding to NCI-H1975 lung cancer cells and their respective cognate sequences within the N-terminally extended PSMGFR sequence. FIG. 82A-82C are FACS graphs showing the percent cells that were recognized by each antibody. FIG. 82D-82F are FACS graphs showing the Mean Fluorescence Intensity, MFI, of each antibody. FIG. 82A and FIG. 82D show the FACS graph of antibodies that were generated by immunizing with the PSMGFR peptide. FIG. 82B and FIG. 82E show the FACS graph of antibodies that were generated by immunizing with the N+20/C-27 peptide. FIG. 82C and FIG. 82F show the FACS graph of antibodies that were generated by immunizing with the N+9/C-9 peptide. FIG. 82G shows the PSMGFR sequence that is extended at the N-terminus by 20 amino acids. FIG. 82A, FIG. 82D show that antibody 20A10 recognizes MUC1* as it exists on H1975 lung cancer cells.

FIG. 83A-83G shows color coded graphs that resulted from FACS analyses of each antibody binding to SKOV-3 ovarian cancer cells and their respective cognate sequences within the N-terminally extended PSMGFR sequence. FIG. 83A-83C are FACS graphs showing the percent cells that were recognized by each antibody. FIG. 83D-83F are FACS graphs showing the Mean Fluorescence Intensity, MFI, of each antibody. FIG. 83A and FIG. 83D show the FACS graph of antibodies that were generated by immunizing with the PSMGFR peptide. FIG. 83B and FIG. 83E show the FACS graph of antibodies that were generated by immunizing with the N+20/C-27 peptide. FIG. 83C and FIG. 83F show the FACS graph of antibodies that were generated by immunizing with the N+9/C-9 peptide. FIG. 83G shows the PSMGFR sequence that is extended at the N-terminus by 20 amino acids. FIG. 83A, FIG. 83D shows that antibody 20A10 recognizes MUC1* as it exists on SKOV-3 ovarian cancer cells.

FIG. 84A-84G shows color coded graphs that resulted from FACS analyses of each antibody binding to DU145 prostate cancer cells and their respective cognate sequences within the N-terminally extended PSMGFR sequence. FIG. 84A-84C are FACS graphs showing the percent cells that were recognized by each antibody. FIG. 84D-84F are FACS graphs showing the Mean Fluorescence Intensity, MFI, of each antibody. FIG. 84A and FIG. 84D show the FACS graph of antibodies that were generated by immunizing with the PSMGFR peptide. FIG. 84B and FIG. 84E show the FACS graph of antibodies that were generated by immunizing with the N+20/C-27 peptide. FIG. 84C and FIG. 84F show the FACS graph of antibodies that were generated by immunizing with the N+9/C-9 peptide. FIG. 84G shows the PSMGFR sequence that is extended at the N-terminus by 20 amino acids. FIG. 84A, FIG. 84E show that antibody 20A10 recognizes MUC1* as it exists on DU145 prostate cancer cells. FIG. 84D, FIG. 84H show that antibody 3C2B1 recognizes MUC1* as it exists on DU145 prostate cancer cells. FIG. 84D, FIG. 84H shows that antibody 5C6F3 recognizes MUC1* as it exists on DU145 prostate cancer cells.

FIG. 85A-85G shows color coded graphs that resulted from FACS analyses of each antibody binding to HPAF-II pancreatic cancer cells and their respective cognate sequences within the N-terminally extended PSMGFR sequence. FIG. 85A-85C are FACS graphs showing the percent cells that were recognized by each antibody. FIG. 85D-85F are FACS graphs showing the Mean Fluorescence Intensity, MFI, of each antibody. FIG. 85A and FIG. 85D show the FACS graph of antibodies that were generated by immunizing with the PSMGFR peptide. FIG. 85B and FIG. 85E show the FACS graph of antibodies that were generated by immunizing with the N+20/C-27 peptide. FIG. 85C and FIG. 85F show the FACS graph of antibodies that were generated by immunizing with the N+9/C-9 peptide. FIG. 85G shows the PSMGFR sequence that is extended at the N-terminus by 20 amino acids. FIG. 85A, FIG. 85D show that antibody 20A10 recognizes MUC1* as it exists on HPAF II pancreatic cancer cells.

FIG. 86A-86G shows color coded graphs that resulted from FACS analyses of each antibody binding to Capan-1 pancreatic cancer cells and their respective cognate sequences within the N-terminally extended PSMGFR sequence. FIG. 86A-86C are FACS graphs showing the percent cells that were recognized by each antibody. FIG. 86D-86F are FACS graphs showing the Mean Fluorescence Intensity, MFI, of each antibody. FIG. 86A and FIG. 86D show the FACS graph of antibodies that were generated by immunizing with the PSMGFR peptide. FIG. 86B and FIG. 86E show the FACS graph of antibodies that were generated by immunizing with the N+20/C-27 peptide. FIG. 86C and FIG. 86F show the FACS graph of antibodies that were generated by immunizing with the N+9/C-9 peptide. FIG. 86G shows the PSMGFR sequence that is extended at the N-terminus by 20 amino acids.

FIG. 87A-87G shows color coded graphs that resulted from FACS analyses of each antibody binding to MDA-MB-231 breast cancer cells, which are nearly MUC1 negative, and their respective cognate sequences within the N-terminally extended PSMGFR sequence. FIG. 87A-87C are FACS graphs showing the percent cells that were recognized by each antibody. FIG. 87D-87F are FACS graphs showing the Mean Fluorescence Intensity, MFI, of each antibody. FIG. 87A and FIG. 87D show the FACS graph of antibodies that were generated by immunizing with the PSMGFR peptide. FIG. 87B and FIG. 87E show the FACS graph of antibodies that were generated by immunizing with the N+20/C-27 peptide. FIG. 87C and FIG. 87F show the FACS graph of antibodies that were generated by immunizing with the N+9/C-9 peptide. FIG. 87G shows the PSMGFR sequence that is extended at the N-terminus by 20 amino acids.

FIG. 88A-88L show photographs of normal liver tissue specimens, each from the same donor but stained with a different antibody of the invention. FIG. 88A-88F show the entire tissue core. FIG. 88G-88L show the 40× magnification of a particular area of the tissue. The tissues are ordered from right to left with antibodies that bind to the most membrane proximal, that is to say most C-terminal portion of the PSMGFR peptide, on the right and antibodies that bind to the most N-terminal portions of the MUC1 extra cellular domain, even beyond the PSMGFR region, on the left. As can be seen in the figure, the most cancer-specific antibodies are those that bind to the more membrane proximal portions of the PSMGFR sequence and antibodies that bind to the most distal, N-terminal portions lose cancer specificity, with those antibodies that bind to epitopes outside of the PSMGFR having lost all cancer specificity. As can be seen, FIGS. 88F and 88L show that antibody 3C2B1, which binds the portion of MUC1* extracellular domain that comprises all or part of the sequence FPFS or PFPFSAQSGA, does not bind to normal liver.

FIG. 89A-89H show photographs of normal heart tissue specimens, stained with different antibodies of the invention. FIG. 89A-89D show the entire tissue core. FIG. 89E-89HL show the 40× magnification of a particular area of the tissue. FIG. 89A and FIG. 89E show staining with MNC2-scFv. FIG. 89B and FIG. 89F show staining with MNE6. FIG. 89C and FIG. 89G show staining with 20A10. FIG. 89D and FIG. 89H show staining with 3C2B1. These antibodies bind to an epitope that comprises all or part of the sequence FPFS or PFPFSAQSGA. All these antibodies are all able to bind to the PSMGFR peptide, bind to the N-10 peptide but do not bind to the C-10 peptide. In addition, these antibodies disrupt the binding of NME7_{AB} to the MUC1* extra cellular domain as exemplified by the PSMGFR peptide. Further, these antibodies recognize a MUC1 cleavage product when the cleavage enzyme is MMP9. As can be seen in the figure, these antibodies show no binding to normal heart tissue. FIG. 89A, FIG. 89E show that reference antibody MNC2 does not bind to normal heart tissue. FIG. 89B, FIG. 89F show that reference antibody MNE6 does not bind to normal heart tissue. FIG. 89C, FIG. 89G show that antibody 20A10 does not bind to normal heart tissue. FIG. 89D, FIG. 89H shows that antibody 3C2B1 does not bind to normal heart tissue.

FIG. 90A-90D show photographs of normal heart tissue specimens, stained with different antibodies of the invention. FIG. 90A-90B show the entire tissue core. FIG. 90C-90D show the 40× magnification of a particular area of the tissue. FIG. 90A and FIG. 90C show staining with MNC3. FIG. 90B and FIG. 90D show staining with 25E6. These antibodies bind to an epitope that comprises all or part of the sequence ASRYNLT. These antibodies are all able to bind to the PSMGFR peptide, bind to the N-10 peptide but also bind to the C-10 peptide. As can be seen, these antibodies are not as cancer-specific and show some binding to normal heart tissue.

FIG. 91A-91B show photographs of normal heart tissue specimens, stained with an antibody of the invention 1E4. FIG. 91A show the entire tissue core. FIG. 91B show the 40× magnification of a particular area of the tissue. Antibody 1E4 binds to an epitope that comprises all or part of the sequence QFNQYKTEA. Antibody 1E4 can bind to the N-10 peptide but also binds to the C-10 peptide. As can be seen in the figure, 1E4 binds to normal heart tissue. As can be seen, these antibodies are not as cancer-specific and show some binding to normal heart tissue.

FIG. 92A-92H show photographs of normal heart tissue specimens, stained with different antibodies of the invention. FIG. 92A-92D show the entire tissue core. FIG. 92E-92HL show the 40× magnification of a particular area of the tissue. FIG. 92A and FIG. 92E show staining with 18B4. FIG. 92B and FIG. 92F show staining with 31A1. FIG. 92C and FIG. 92G show staining with 32C1. FIG. 92D and FIG. 92H show staining with 29H1. These antibodies bind to an epitope that comprises all or part of the sequence GTINVHDVET, which is the most N-terminal part of the PSMGFR peptide. None of these antibodies are able to bind to the N-10 peptide. As can be seen in the figure, all of these antibodies except 18B4 show binding to normal heart tissue.

FIG. 93A-93D show photographs of normal heart tissue specimens, stained with antibodies of the invention. FIG. 93A-93B show the entire tissue core. FIG. 93C-93D show the 40× magnification of a particular area of the tissue. FIG. 93A and FIG. 93C show staining with antibody 8A9. FIG. 93B and FIG. 93D show staining with antibody 17H6. Both antibodies bind to an epitope that that is outside of the PSMGFR region and comprises all or part of the sequence VQLTLAFRE. As can be seen in the figure, both antibodies show strong binding to normal heart tissue.

FIG. 94A-94B show photographs of normal heart tissue specimens, stained with an antibody of the invention 45C11. FIG. 94A show the entire tissue core. FIG. 94B show the 40× magnification of a particular area of the tissue. Antibody 45C11 binds to an epitope that is outside of the PSMGFR region and comprises all or part of the sequence SNIKFRPGSVV. Antibody 45C11 cannot bind to the N-10 peptide. As can be seen in the figure, 45C11 binds strongly to normal heart tissue.

FIG. 95A-95H show photographs of normal liver tissue specimens, stained with different antibodies of the invention. FIG. 95A-95D show the entire tissue core. FIG. 95E-95HL show the 40× magnification of a particular area of the tissue. FIG. 95A and FIG. 95E show staining with reference antibody MNC2-scFv. FIG. 95B and FIG. 95F show staining with reference antibody MNE6. FIG. 95C and FIG. 95G show staining with 20A10. FIG. 95D and FIG. 95H show staining with 3C2B1. These antibodies bind to an epitope that comprises all or part of the sequence FPFS or PFPFSAQSGA. All these antibodies are all able to bind to the PSMGFR peptide, bind to the N-10 peptide but do not bind to the C-10 peptide. In addition, these antibodies disrupt the binding of NME7_{AB} to the MUC1* extra cellular domain as exemplified by the PSMGFR peptide. Further, these antibodies recognize a MUC1 cleavage product when the cleavage enzyme is MMP9. As can be seen in the figure, these antibodies show no binding to normal liver tissue.

FIG. 96A-96D show photographs of normal liver tissue specimens, stained with different antibodies of the invention. FIG. 96A-96B show the entire tissue core. FIG. 96C-96D show the 40× magnification of a particular area of the tissue. FIG. 96A and FIG. 96C show staining with MNC3. FIG. 96B and FIG. 96D show staining with 25E6. These antibodies bind to an epitope that comprises all or part of the sequence ASRYNLT. These antibodies are all able to bind to the PSMGFR peptide, bind to the N-10 peptide but also bind to the C-10 peptide. As can be seen, these antibodies are not as cancer-specific and show some binding to normal liver tissue.

FIG. 97A-97B show photographs of normal liver tissue specimens, stained with an antibody of the invention 1E4. FIG. 97A show the entire tissue core. FIG. 97B show the 40× magnification of a particular area of the tissue. Antibody 1E4 binds to an epitope that comprises all or part of the sequence QFNQYKTEA. Antibody 1E4 can bind to the N-10 peptide but also binds to the C-10 peptide. As can be seen in the figure, 1E4 binds to normal liver tissue.

FIG. 98A-98H show photographs of normal liver tissue specimens, stained with different antibodies of the invention. FIG. 98A-98D show the entire tissue core. FIG. 98E-98H show the 40× magnification of a particular area of the tissue. FIG. 98A and FIG. 98E show staining with 18B4. FIG. 98B and FIG. 98F show staining with 31A1. FIG. 98C and FIG. 98G show staining with 32C1. FIG. 98D and FIG. 98H show staining with 29H1. These antibodies bind to an epitope that comprises all or part of the sequence GTINVHDVET, which is the most N-terminal part of the PSMGFR peptide. None of these antibodies are able to bind to the N-10 peptide. As can be seen in the figure, 32C1 shows some binding to normal liver and 29H1 shows extremely strong binding to normal liver tissue.

FIG. 99A-99D show photographs of normal liver tissue specimens, stained with antibodies of the invention. FIG. 99A-99B show the entire tissue core. FIG. 99C-99D show the 40× magnification of a particular area of the tissue. FIG. 99A and FIG. 99C show staining with antibody 8A9. FIG. 99B and FIG. 99D show staining with antibody 17H6. Both antibodies bind to an epitope that that is outside of the PSMGFR region and comprises all or part of the sequence VQLTLAFRE. As can be seen in the figure, 8A9 shows strong binding to normal liver tissue. 17H6 is a weak antibody and it is possible that it was not used at a high enough concentration in this study.

FIG. 100A-100B show photographs of normal liver tissue specimens, stained with an antibody of the invention 45C11. FIG. 100A show the entire tissue core. FIG. 100B show the 40× magnification of a particular area of the tissue. Antibody 45C11 binds to an epitope that is outside of the PSMGFR region and comprises all or part of the sequence SNIKFRPGSVV. Antibody 45C11 cannot bind to the N-10 peptide. As can be seen in the figure, 45C11 binds strongly to normal liver tissue.

FIG. 101A-101H show photographs of normal lung tissue specimens, stained with different antibodies of the invention. FIG. 101A-11D show the entire tissue core. FIG. 101E-101H show the 40× magnification of a particular area of the tissue. FIG. 101A and FIG. 101E show staining with MNC2-scFv. FIG. 101B and FIG. 101F show staining with MNE6. FIG. 101C and FIG. 101G show staining with 20A10. FIG. 101D and FIG. 101H show staining with 3C2B1. These antibodies bind to an epitope that comprises all or part of the sequence FPFS or PFPFSAQSGA. All these antibodies are all able to bind to the PSMGFR peptide, bind to the N-10 peptide but do not bind to the C-10 peptide. In addition, these antibodies disrupt the binding of NME7_{AB} to the MUC1* extra cellular domain as exemplified by the PSMGFR peptide. Further, these antibodies recognize a MUC1 cleavage product when the cleavage enzyme is MMP9. As can be seen in the figure, these antibodies show no binding to normal lung tissue.

FIG. 102A-102D show photographs of normal lung tissue specimens, stained with different antibodies of the invention. FIG. 102A-102B show the entire tissue core. FIG. 102C-102D show the 40× magnification of a particular area of the tissue. FIG. 102A and FIG. 102C show staining with MNC3. FIG. 102B and FIG. 102D show staining with 25E6. These antibodies bind to an epitope that comprises all or part of the sequence ASRYNLT. These antibodies are all able to bind to the PSMGFR peptide, bind to the N-10 peptide but also bind to the C-10 peptide. As can be seen, these antibodies are not as cancer-specific and show some binding to normal lung tissue.

FIG. 103A-103B show photographs of normal lung tissue specimens, stained with an antibody of the invention 1E4. FIG. 103A show the entire tissue core. FIG. 103B show the 40× magnification of a particular area of the tissue. Antibody 1E4 binds to an epitope that comprises all or part of the sequence QFNQYKTEA. Antibody 1E4 can bind to the N-10 peptide but also binds to the C-10 peptide.

FIG. 104A-104H show photographs of normal lung tissue specimens, stained with different antibodies of the invention. FIG. 104A-104D show the entire tissue core. FIG. 104E-104H show the 40× magnification of a particular area of the tissue. FIG. 104A and FIG. 104E show staining with 18B4. FIG. 104B and FIG. 104F show staining with 31A1. FIG. 104C and FIG. 104G show staining with 32C1. FIG. 104D and FIG. 104H show staining with 29H1. These antibodies bind to an epitope that comprises all or part of the sequence GTINVHDVET, which is the most N-terminal part of the PSMGFR peptide. None of these antibodies are able to bind to the N-10 peptide. As can be seen in the figure, all these antibodies show strong binding to normal lung tissue.

FIG. 105A-105D show photographs of normal lung tissue specimens, stained with antibodies of the invention. FIG. 105A-105B show the entire tissue core. FIG. 105C-105D show the 40× magnification of a particular area of the tissue. FIG. 105A and FIG. 105C show staining with antibody 8A9. FIG. 105B and FIG. 105D show staining with antibody 17H6. Both antibodies bind to an epitope that that is outside of the PSMGFR region and comprises all or part of the sequence VQLTLAFRE. As can be seen in the figure, 8A9 shows strong binding to normal lung tissue. 17H6 is a weak antibody and it is possible that it was not used at a high enough concentration in this study.

FIG. 106A-106B show photographs of normal lung tissue specimens, stained with an antibody of the invention 45C11. FIG. 106A show the entire tissue core. FIG. 106B show the 40× magnification of a particular area of the tissue. Antibody 45C11 binds to an epitope that is outside of the PSMGFR region and comprises all or part of the sequence SNIKFRPGSVV. Antibody 45C11 cannot bind to the N-10 peptide. As can be seen in the figure, 45C11 binds to normal lung tissue.

FIG. 107A-107H show photographs of normal bone marrow tissue specimens, stained with different antibodies of the invention. FIG. 107A-107D show the entire tissue core. FIG. 107E-107H show the 40× magnification of a particular area of the tissue. FIG. 107A and FIG. 107E show staining with MNC2-scFv. FIG. 107B and FIG. 107F show staining with MNE6. FIG. 107C and FIG. 107G show staining with 20A10. FIG. 107D and FIG. 107H show staining with 3C2B1. These antibodies bind to an epitope that comprises all or part of the sequence FPFS or PFPFSAQSGA. All these antibodies are all able to bind to the PSMGFR peptide, bind to the N-10 peptide but do not bind to the C-10 peptide. In addition, these antibodies disrupt the binding of NME7_{AB} to the MUC1* extra cellular domain as exemplified by the PSMGFR peptide. Further, these antibodies recognize a MUC1 cleavage product when the cleavage enzyme is MMP9. As can be seen in the figure, these antibodies show no binding to normal bone marrow tissue.

FIG. 108A-108D show photographs of normal bone marrow tissue specimens, stained with different antibodies of the invention. FIG. 108A-108B show the entire tissue core. FIG. 108C-108D show the 40× magnification of a particular area of the tissue. FIG. 108A and FIG. 108C show staining with MNC3. FIG. 108B and FIG. 108D show staining with 25E6. These antibodies bind to an epitope that comprises all or part of the sequence ASRYNLT. These antibodies are all able to bind to the PSMGFR peptide, bind to the N-10 peptide but also bind to the C-10 peptide.

FIG. 109A-109B show photographs of normal bone marrow tissue specimens, stained with an antibody of the invention 1E4. FIG. 109A show the entire tissue core. FIG. 109B show the 40× magnification of a particular area of the tissue. Antibody 1E4 binds to an epitope that comprises all or part of the sequence QFNQYKTEA. Antibody 1E4 can bind to the N-10 peptide but also binds to the C-10 peptide. 1E4 binds to normal bone marrow.

FIG. 110A-110H show photographs of normal bone marrow tissue specimens, stained with different antibodies of the invention. FIG. 110A-110D show the entire tissue core. FIG. 110E-110H show the 40× magnification of a particular area of the tissue. FIG. 110A and FIG. 110E show staining with 18B4. FIG. 110B and FIG. 110F show staining with 31A1. FIG. 110C and FIG. 110G show staining with 32C1. FIG. 110D and FIG. 110H show staining with 29H1. These antibodies bind to an epitope that comprises all or part of the sequence GTINVHDVET, which is the most N-terminal part of the PSMGFR peptide. None of these antibodies are able to bind to the N-10 peptide. As can be seen in the figure, all these antibodies show strong binding to normal bone marrow tissue.

FIG. 111A-111D show photographs of normal bone marrow tissue specimens, stained with antibodies of the invention. FIG. 111A-111B show the entire tissue core. FIG. 111C-111D show the 40× magnification of a particular area of the tissue. FIG. 111A and FIG. 111C show staining with antibody 8A9. FIG. 111B and FIG. 111D show staining with antibody 17H6. Both antibodies bind to an epitope that that is outside of the PSMGFR region and comprises all or part of the sequence VQLTLAFRE. As can be seen in the figure, 8A9 shows strong binding to normal bone marrow tissue. 17H6 is a weak antibody and it is possible that it was not used at a high enough concentration in this study.

FIG. 112A-112B show photographs of normal bone marrow tissue specimens, stained with an antibody of the invention 45C11. FIG. 112A show the entire tissue core. FIG. 112B show the 40× magnification of a particular area of the tissue. Antibody 45C11 binds to an epitope that is outside of the PSMGFR region and comprises all or part of the sequence SNIKFRPGSVV. Antibody 45C11 cannot bind to the N-10 peptide. As can be seen in the figure, 45C11 binds to normal bone marrow tissue.

FIG. 113A-113C shows photographs, array map and description of FDA normal tissue array MNO1021 stained with the anti-PSMGFR antibody 20A10 at 0.25 ug/mL. FIG. 113A shows photographs of the tissue micro array. FIG. 113B shows map of the array with abbreviated tissue descriptors. FIG. 113C detailed description of the tissue micro array with non-identifying donor data.

FIG. 114A-114X shows photographs of specific tissues from FDA normal tissue array MNO1021 stained with the anti-PSMGFR antibody 20A10 at 0.25 ug/mL, magnified to 6× and 20×. FIG. 114A and FIG. 114E are adrenal gland. FIG. 114B and FIG. 114F are breast. FIG. 114C and FIG. 114G are fallopian tube. FIG. 114D and FIG. 114H are kidney. FIG. 114I and FIG. 114M are heart muscle. FIG. 114J and FIG. 114N are liver. FIG. 114K and FIG. 114O are lung. FIG. 114L and FIG. 114P are ureter. FIG. 114Q and FIG. 114U are eye. FIG. 114R and FIG. 114V are cerebral cortex. FIG. 114S and FIG. 114W are bone marrow. FIG. 114T and FIG. 114X are skeletal muscle.

FIG. 115A-115C shows photographs, array map and description of breast cancer tissue array BR1141 stained with the anti-PSMGFR antibody 20A10 at 0.25 ug/mL. FIG. 115A shows photographs of the tissue micro array. FIG. 115B shows map of the array with abbreviated tissue descriptors. FIG. 115C detailed description of the tissue micro array with non-identifying donor data.

FIG. 116A-116F shows photographs of specific tissues from breast cancer tissue array BR1141 stained with the anti-PSMGFR antibody 20A10 at 0.25 ug/mL, magnified to 6× and 20×. FIG. 116A and FIG. 116D are photographs of a Grade 2 invasive ductal carcinoma. FIG. 116B and FIG. 116E are photographs of a Grade 2 invasive ductal carcinoma. FIG. 116C and FIG. 116F are photographs of a Grade 2 invasive ductal carcinoma.

FIG. 117A-117C shows photographs, array map and description of pancreatic cancer tissue array PA805c stained with the anti-PSMGFR antibody 20A10 at 0.25 ug/mL. FIG. 117A shows photographs of the tissue micro array. FIG. 117B shows map of the array with abbreviated tissue descriptors. FIG. 117C detailed description of the tissue micro array with non-identifying donor data.

FIG. 118A-118F shows photographs of specific tissues from pancreatic cancer tissue array PA805c stained with the anti-PSMGFR antibody 20A10 at 0.25 ug/mL, magnified to 6× and 20×. FIG. 118A and FIG. 118D are photographs of a Grade 2 papillary adenocarcinoma. FIG. 118B and FIG. 118E are photographs of a Grade 2-3 ductal carcinoma.

FIG. 118C and FIG. 118F are photographs of a Grade 3 invasive adenocarcinoma.

FIG. 119A-119C shows photographs, array map and description of esophageal cancer tissue array BC001113 stained with the anti-PSMGFR antibody 20A10 at 0.25 ug/mL. FIG. 119A shows photographs of the tissue micro array. FIG. 119B shows map of the array with abbreviated tissue descriptors. FIG. 119C detailed description of the tissue micro array with non-identifying donor data.

FIG. 120A-120F shows photographs of specific tissues from esophageal cancer tissue array BC001113 stained with the anti-PSMGFR antibody 20A10 at 0.25 ug/mL, magnified to 6× and 20×. FIG. 120A and FIG. 120D are photographs of the specimen at position A1. FIG. 120B and FIG. 120E are photographs of the specimen at position A7. FIG. 120C and FIG. 120F are photographs of the specimen at position A8.

FIG. 121A-121C shows photographs, array map and description of FDA normal tissue array MNO1021 stained with the anti-PSMGFR antibody 3C2B1 at 20 ug/mL. FIG. 121A shows photographs of the tissue micro array. FIG. 121B shows map of the array with abbreviated tissue descriptors. FIG. 121C detailed description of the tissue micro array with non-identifying donor data.

FIG. 122A-122X shows photographs of specific tissues from FDA normal tissue array MNO1021 stained with the anti-PSMGFR antibody 3C2B1 at 20 ug/mL, magnified to 6× and 20×. FIG. 122A and FIG. 122E are adrenal gland. FIG. 122B and FIG. 122F are breast. FIG. 122C and FIG. 122G are fallopian tube. FIG. 122D and FIG. 122H are kidney. FIG. 122I and FIG. 122M are heart muscle. FIG. 122J and FIG. 122N are liver. FIG. 122K and FIG. 122O are lung. FIG. 122L and FIG. 122P are ureter. FIG. 122Q and FIG. 122U are eye. FIG. 122R and FIG. 122V are cerebral cortex. FIG. 122S and FIG. 122W are bone marrow. FIG. 122T and FIG. 122X are skeletal muscle.

FIG. 123A-123C shows photographs, array map and description of pancreatic cancer tissue array PA1003 stained with the anti-PSMGFR antibody 3C2B1 at 20 ug/mL. FIG. 123A shows photographs of the tissue micro array. FIG. 123B shows map of the array with abbreviated tissue descriptors. FIG. 123C detailed description of the tissue micro array with non-identifying donor data.

FIG. 124A-124F shows photographs of specific tissues from pancreatic cancer tissue array PA1003 stained with the anti-PSMGFR antibody 3C2B1 at 20 ug/mL, magnified to 6× and 20×. FIG. 124A and FIG. 124D are photographs of a Grade 2 adenocarcinoma. FIG. 124B and FIG. 124E are photographs of a Grade 2 adenocarcinoma. FIG. 124C and FIG. 124F are photographs of a Grade 2 adenocarcinoma.

FIG. 125A-125C shows photographs, array map and description of breast cancer tissue array BR1141 stained with the anti-PSMGFR antibody 3C2B1 at 20 ug/mL. FIG. 125A shows photographs of the tissue micro array. FIG. 125B shows map of the array with abbreviated tissue descriptors. FIG. 125C detailed description of the tissue micro array with non-identifying donor data.

FIG. 126A-126F shows photographs of specific tissues from breast cancer tissue array BR1141 stained with the anti-PSMGFR antibody 3C2B1 at 20 ug/mL, magnified to 6× and 20×. FIG. 126A and FIG. 126D are photographs of a Grade 2 invasive ductal carcinoma. FIG. 126B and FIG. 126E are photographs of a Grade 2 invasive ductal carcinoma. FIG. 126C and FIG. 126F are photographs of a Grade 2 invasive carcinoma.

FIG. 127A-127C shows photographs, array map and description of FDA normal tissue array MNO1021 stained with the anti-PSMGFR antibody 5C6F3 at 1 ug/mL. FIG. 127A shows photographs of the tissue micro array. FIG. 127B shows map of the array with abbreviated tissue descriptors. FIG. 127C detailed description of the tissue micro array with non-identifying donor data.

FIG. 128A-128X shows photographs of specific tissues from FDA normal tissue array MNO1021 stained with the anti-PSMGFR antibody 5C6F3 at 1 ug/mL, magnified to 6× and 20×. FIG. 128A and FIG. 128E are adrenal gland. FIG. 128B and FIG. 128F are breast. FIG. 128C and FIG. 128G are fallopian tube. FIG. 128D and FIG. 128H are kidney. FIG. 128I and FIG. 128M are heart muscle. FIG. 128J and FIG. 128N are liver. FIG. 128K and FIG. 128O are lung. FIG. 128L and FIG. 128P are ureter. FIG. 128Q and FIG. 128U are eye. FIG. 128R and FIG. 128V are cerebral cortex. FIG. 128S and FIG. 128W are bone marrow. FIG. 128T and FIG. 128X are skeletal muscle.

FIG. 129A-129C shows photographs, array map and description of pancreatic cancer tissue array PA1003 stained with the anti-PSMGFR antibody 5C6F3 at 1-20 ug/mL. FIG. 129A shows photographs of the tissue micro array. FIG. 129B shows map of the array with abbreviated tissue descriptors. FIG. 129C detailed description of the tissue micro array with non-identifying donor data.

FIG. 130A-130F shows photographs of specific tissues from pancreatic cancer tissue array PA1003 stained with the anti-PSMGFR antibody 5C6F3 at 1 ug/mL, magnified to 6× and 20×. FIG. 130A and FIG. 130D are photographs of a Grade 2 adenocarcinoma. FIG. 130B and FIG. 130E are photographs of a Grade 2 adenocarcinoma. FIG. 130C and FIG. 130F are photographs of a Grade 2 adenocarcinoma.

FIG. 131A-131C shows photographs, array map and description of breast cancer tissue array BR1141 stained with the anti-PSMGFR antibody 5C6F3 at 1 ug/mL. FIG. 131A shows photographs of the tissue micro array. FIG. 131B shows map of the array with abbreviated tissue descriptors. FIG. 131C detailed description of the tissue micro array with non-identifying donor data.

FIG. 132A-132F shows photographs of specific tissues from breast cancer tissue array BR1141 stained with the anti-PSMGFR antibody 5C6F3 at 1 ug/mL, magnified to 6× and 20×. FIG. 132A and FIG. 132D are photographs of a Grade 2 invasive ductal carcinoma. FIG. 132B and FIG. 132E are photographs of a Grade 2 invasive ductal carcinoma. FIG. 132C and FIG. 132F are photographs of a Grade 2 invasive carcinoma.

FIG. 133A-133C shows photographs, array map and description of FDA normal tissue array MNO1021 stained with the anti-PSMGFR antibody 18B4 at 10 ug/mL. FIG. 133A shows photographs of the tissue micro array. FIG. 133B shows map of the array with abbreviated tissue descriptors. FIG. 133C detailed description of the tissue micro array with non-identifying donor data.

FIG. 134A-134X shows photographs of specific tissues from FDA normal tissue array MNO1021 stained with the anti-PSMGFR antibody 18B4 at 10 ug/mL, magnified to 6× and 20×. FIG. 134A and FIG. 134E are adrenal gland. FIG. 134B and FIG. 134F are breast. FIG. 134C and FIG. 134G are fallopian tube. FIG. 134D and FIG. 134H are kidney. FIG. 134I and FIG. 134M are heart muscle. FIG. 134J and FIG. 134N are liver. FIG. 134K and FIG. 134O are lung. FIG. 134L and FIG. 134P are ureter. FIG. 134Q and FIG. 134U are eye. FIG. 134R and FIG. 134V are cerebral cortex. FIG. 134S and FIG. 134W are bone marrow. FIG. 134T and FIG. 134X are skeletal muscle.

FIG. 135A-135C shows photographs, array map and description of breast cancer tissue array BR1141 stained with the anti-PSMGFR antibody 18B4 at 10 ug/mL. FIG. 135A shows photographs of the tissue micro array. FIG. 135B shows map of the array with abbreviated tissue descriptors. FIG. 135C detailed description of the tissue micro array with non-identifying donor data.

FIG. 136A-136F shows photographs of specific tissues from breast cancer tissue array BR1141 stained with the anti-PSMGFR antibody 18B4 at 10 ug/mL, magnified to 6× and 20×. FIG. 136A and FIG. 136D are photographs of a Grade 2 invasive ductal carcinoma. FIG. 136B and FIG. 136E are photographs of a Grade 2 invasive ductal carcinoma. FIG. 136C and FIG. 136F are photographs of a Grade 2 invasive ductal carcinoma.

FIG. 137A-137C shows photographs, array map and description of esophageal cancer tissue array BC001113 stained with the anti-PSMGFR antibody 18B4 at 10 ug/mL. FIG. 137A shows photographs of the tissue micro array. FIG. 137B shows map of the array with abbreviated tissue descriptors. FIG. 137C detailed description of the tissue micro array with non-identifying donor data.

FIG. 138A-138F shows photographs of specific tissues from esophageal cancer tissue array BC001113 stained with the anti-PSMGFR antibody 18B4 at 10 ug/mL, magnified to 6× and 20×. FIG. 138A and FIG. 138D are photographs of the specimen at position A1. FIG. 138B and FIG. 138E are photographs of the specimen at position A7. FIG. 138C and FIG. 138F are photographs of the specimen at position A8.

FIG. 139A-139C shows photographs, array map and description of FDA normal tissue array MNO1021 stained with the anti-PSMGFR antibody 18G12 at 10 ug/mL. FIG. 139A shows photographs of the tissue micro array. FIG. 139B shows map of the array with abbreviated tissue descriptors. FIG. 139C detailed description of the tissue micro array with non-identifying donor data.

FIG. 140A-140X shows photographs of specific tissues from FDA normal tissue array MNO1021 stained with the anti-PSMGFR antibody 18G12 at 10 ug/mL, magnified to 6× and 20×. FIG. 140A and FIG. 140E are adrenal gland. FIG. 140B and FIG. 140F are breast. FIG. 140C and FIG. 140G are fallopian tube. FIG. 140D and FIG. 140H are kidney. FIG. 140I and FIG. 140M are heart muscle. FIG. 140J and FIG. 140N are liver. FIG. 140K and FIG. 140O are lung. FIG. 140L and FIG. 140P are ureter. FIG. 140Q and FIG. 140U are eye. FIG. 140R and FIG. 140V are cerebral cortex. FIG. 140S and FIG. 140W are bone marrow. FIG. 140T and FIG. 140X are skeletal muscle.

FIG. 141A-141C shows photographs, array map and description of breast cancer tissue array BR1141 stained with the anti-PSMGFR antibody 18G12 at 15 ug/mL. FIG. 141A shows photographs of the tissue micro array. FIG. 141B shows map of the array with abbreviated tissue descriptors. FIG. 141C detailed description of the tissue micro array with non-identifying donor data.

FIG. 142A-142F shows photographs of specific tissues from breast cancer tissue array BR1141 stained with the anti-PSMGFR antibody 18G12 at 15 ug/mL, magnified to 6× and 20×. FIG. 142A and FIG. 142D are photographs of a Grade 2 invasive ductal carcinoma. FIG. 142B and FIG. 142E are photographs of a Grade 2 invasive ductal carcinoma. FIG. 142C and FIG. 142F are photographs of a Grade 2 invasive ductal carcinoma.

FIG. 143A-143C shows photographs, array map and description of pancreatic cancer tissue array PA1003 stained with the anti-PSMGFR antibody 18G12 at 15 ug/mL. FIG. 143A shows photographs of the tissue micro array. FIG. 143B shows map of the array with abbreviated tissue descriptors. FIG. 143C detailed description of the tissue micro array with non-identifying donor data.

FIG. 144A-144F shows photographs of specific tissues from pancreatic cancer tissue array PA1003 stained with the anti-PSMGFR antibody 18G12 at 15 ug/mL, magnified to 6× and 20×. FIG. 144A and FIG. 144D are photographs of a Grade 2 adenocarcinoma. FIG. 144B and FIG. 144E are photographs of a Grade 2 adenocarcinoma. FIG. 144C and FIG. 144F are photographs of a Grade 2-3 adenocarcinoma with lymph node involvement.

FIG. 145A-145C shows photographs, array map and description of esophageal cancer tissue array BC001113 stained with the anti-PSMGFR antibody 18G12 at 30 ug/mL. FIG. 145A shows photographs of the tissue micro array. FIG. 145B shows map of the array with abbreviated tissue descriptors. FIG. 145C detailed description of the tissue micro array with non-identifying donor data.

FIG. 146A-146F shows photographs of specific tissues from esophageal cancer tissue array BC001113 stained with the anti-PSMGFR antibody 18G12 at 30 ug/mL, magnified to 6× and 20×. FIG. 146A and FIG. 146D are photographs of the specimen at position A1. FIG. 146B and FIG. 146E are photographs of the specimen at position A7. FIG. 146C and FIG. 146F are photographs of the specimen at position A8.

FIG. 147A-147C shows photographs, array map and description of FDA normal tissue array MNO1021 stained with the anti-PSMGFR antibody 25E6 at 5.0 ug/mL. FIG. 147A shows photographs of the tissue micro array. FIG. 147B shows map of the array with abbreviated tissue descriptors. FIG. 147C detailed description of the tissue micro array with non-identifying donor data.

FIG. 148A-148X shows photographs of specific tissues from FDA normal tissue array 1021 stained with the anti-PSMGFR antibody 25E6 at 5.0 ug/mL, magnified to 6× and 20×. FIG. 148A and FIG. 148E are adrenal gland. FIG. 148B and FIG. 148F are breast. FIG. 148C and FIG. 148G are fallopian tube. FIG. 148D and FIG. 148H are kidney. FIG. 148I and FIG. 148M are heart muscle. FIG. 148J and FIG. 148N are liver. FIG. 148K and FIG. 148O are lung. FIG. 148L and FIG. 148P are ureter. FIG. 148Q and FIG. 148U are eye. FIG. 148R and FIG. 148V are cerebral cortex. FIG. 148S and FIG. 148W are bone marrow. FIG. 148T and FIG. 148X are skeletal muscle.

FIG. 149A-149C shows photographs, array map and description of breast cancer tissue array BR1141 stained with the anti-PSMGFR antibody 25E6 at 5.0 ug/mL. FIG. 149A shows photographs of the tissue micro array. FIG. 149B shows map of the array with abbreviated tissue descriptors. FIG. 149C detailed description of the tissue micro array with non-identifying donor data.

FIG. 150A-150F shows photographs of specific tissues from breast cancer tissue array BR1141 stained with the anti-PSMGFR antibody 25E6 at 5.0 ug/mL, magnified to 6× and 20×. FIG. 150A and FIG. 150D are photographs of a Grade 2 invasive ductal carcinoma. FIG. 150B and FIG. 150E are photographs of a Grade 2 invasive ductal carcinoma. FIG. 150C and FIG. 150F are photographs of a Grade 2 invasive ductal carcinoma.

FIG. 151A-151C shows photographs, array map and description of pancreatic cancer tissue array PA1003 stained with the anti-PSMGFR antibody 25E6 at 5.0 ug/mL. FIG. 151A shows photographs of the tissue micro array. FIG. 151B shows map of the array with abbreviated tissue descriptors. FIG. 151C detailed description of the tissue micro array with non-identifying donor data.

FIG. 152A-152F shows photographs of specific tissues from pancreatic cancer tissue array PA1003 stained with the anti-PSMGFR antibody 25E6 at 5.0 ug/mL, magnified to 6× and 20×. FIG. 152A and FIG. 152D are photographs of a Grade 2 adenocarcinoma. FIG. 152B and FIG. 152E are photographs of a Grade 1 adenocarcinoma. FIG. 152C and FIG. 152F are photographs of a Grade 1 adenocarcinoma.

FIG. 153A-153C shows photographs, array map and description of FDA normal tissue array MNO1021 stained with the anti-PSMGFR antibody 28F9 at 15.0 ug/mL. FIG. 153A shows photographs of the tissue micro array. FIG. 153B shows map of the array with abbreviated tissue descriptors. FIG. 153C detailed description of the tissue micro array with non-identifying donor data.

FIG. 154A-154X shows photographs of specific tissues from FDA normal tissue array MNO1021 stained with the anti-PSMGFR antibody 28F9 at 15.0 ug/mL, magnified to 6× and 20×. FIG. 154A and FIG. 154E are adrenal gland. FIG. 154B and FIG. 154F are breast. FIG. 154C and FIG. 154G are fallopian tube. FIG. 154D and FIG. 154H are kidney. FIG. 154I and FIG. 154M are heart muscle. FIG. 154J and FIG. 154N are liver. FIG. 154K and FIG. 154O are lung. FIG. 154L and FIG. 154P are ureter. FIG. 154Q and FIG. 154U are eye. FIG. 154R and FIG. 154V are cerebral cortex. FIG. 154S and FIG. 154W are bone marrow. FIG. 154T and FIG. 154X are skeletal muscle.

FIG. 155A-155C shows photographs, array map and description of breast cancer tissue array BR1141 stained with the anti-PSMGFR antibody 28F9 at 15.0 ug/mL. FIG. 155A shows photographs of the tissue micro array. FIG. 155B shows map of the array with abbreviated tissue descriptors. FIG. 155C detailed description of the tissue micro array with non-identifying donor data.

FIG. 156A-156F shows photographs of specific tissues from breast cancer tissue array BR1141 stained with the anti-PSMGFR antibody 28F9 at 15.0 ug/mL, magnified to 6× and 20×. FIG. 156A and FIG. 156D are photographs of a Grade 2 invasive ductal carcinoma. FIG. 156B and FIG. 156E are photographs of a Grade 2 invasive ductal carcinoma. FIG. 156C and FIG. 156F are photographs of a Grade 2 invasive ductal carcinoma.

FIG. 157A-157C shows photographs, array map and description of FDA normal tissue array MNO1021 stained with the N+20/C-27 antibody 1E4 at 7.5 ug/mL. FIG. 157A shows photographs of the tissue micro array. FIG. 157B shows map of the array with abbreviated tissue descriptors. FIG. 157C detailed description of the tissue micro array with non-identifying donor data.

FIG. 158A-158X shows photographs of specific tissues from FDA normal tissue array MNO1021 stained with the N+20/C-27 antibody 1E4 at 7.5 ug/mL, magnified to 6× and 20×. FIG. 158A and FIG. 158E are adrenal gland. FIG. 158B and FIG. 158F are breast.

FIG. 158C and FIG. 158G are fallopian tube. FIG. 158D and FIG. 158H are kidney. FIG. 158I and FIG. 158M are heart muscle. FIG. 158J and FIG. 158N are liver. FIG. 158K and FIG. 158O are lung. FIG. 158L and FIG. 158P are ureter. FIG. 158Q and FIG. 158U are eye. FIG. 158R and FIG. 158V are cerebral cortex. FIG. 158S and FIG. 158W are bone marrow. FIG. 158T and FIG. 158X are skeletal muscle.

FIG. 159A-159C shows photographs, array map and description of breast cancer tissue array BR1007 stained with the N+20/C-27 antibody 1E4 at 10.0 ug/mL. FIG. 159A shows photographs of the tissue micro array. FIG. 159B shows map of the array with abbreviated tissue descriptors. FIG. 159C detailed description of the tissue micro array with non-identifying donor data.

FIG. 160A-160F shows photographs of specific tissues from breast cancer tissue array BR1007 stained with the N+20/C-27 antibody 1E4 at 10.0 ug/mL, magnified to 6× and 20×. FIG. 160A and FIG. 160D are photographs of a Grade 2 invasive ductal carcinoma with positive lymph nodes. FIG. 160B and FIG. 160E are photographs of a Grade 2 invasive ductal carcinoma. FIG. 160C and FIG. 160F are photographs of a Grade 2 invasive ductal carcinoma.

FIG. 161A-161C shows photographs, array map and description of FDA normal tissue array MNO1021 stained with the N+20/C-27 antibody 29H1 at 0.5 ug/mL. FIG. 161A shows photographs of the tissue micro array. FIG. 161B shows map of the array with abbreviated tissue descriptors. FIG. 161C detailed description of the tissue micro array with non-identifying donor data.

FIG. 162A-162X shows photographs of specific tissues from FDA normal tissue array MNO1021 stained with the N+20/C-27 antibody 29H1 at 0.5 ug/mL, magnified to 6× and 20×. FIG. 162A and FIG. 162E are adrenal gland. FIG. 162B and FIG. 162F are breast.

FIG. 162C and FIG. 162G are fallopian tube. FIG. 162D and FIG. 162H are kidney. FIG. 162I and FIG. 162M are heart muscle. FIG. 162J and FIG. 162N are liver. FIG. 162K and FIG. 162O are lung. FIG. 162L and FIG. 162P are ureter. FIG. 162Q and FIG. 162U are eye. FIG. 162R and FIG. 162V are cerebral cortex. FIG. 162S and FIG. 162W are bone marrow. FIG. 162T and FIG. 162X are skeletal muscle.

FIG. 163A-163C shows photographs, array map and description of breast cancer tissue array BR1141 stained with the N+20/C-27 antibody 29H1 at 0.5 ug/mL. FIG. 163A shows photographs of the tissue micro array. FIG. 163B shows map of the array with abbreviated tissue descriptors. FIG. 163C detailed description of the tissue micro array with non-identifying donor data.

FIG. 164A-164F shows photographs of specific tissues from breast cancer tissue array BR1141 stained with the N+20/C-27 antibody 29H1 at 0.5 ug/mL, magnified to 6× and 20×. FIG. 164A and FIG. 164D are photographs of a Grade 2 invasive ductal carcinoma. FIG. 164B and FIG. 164E are photographs of a Grade 2 invasive ductal carcinoma. FIG. 164C and FIG. 164F are photographs of a Grade 2 invasive ductal carcinoma.

FIG. 165A-165C shows photographs, array map and description of pancreatic cancer tissue array PA1003 stained with the N+20/C-27 antibody 29H1 at 0.5 ug/mL. FIG. 165A shows photographs of the tissue micro array. FIG. 165B shows map of the array with abbreviated tissue descriptors. FIG. 165C detailed description of the tissue micro array with non-identifying donor data.

FIG. 166A-166F shows photographs of specific tissues from pancreatic cancer tissue array PA1003 stained with the N+20/C-27 antibody 29H1 at 0.5 ug/mL, magnified to 6× and 20×. FIG. 166A and FIG. 166D are photographs of a Grade 2 adenocarcinoma. FIG. 166B and FIG. 166E are photographs of a Grade 2 adenocarcinoma. FIG. 166C and FIG. 166F are photographs of a Grade 3 adenocarcinoma.

FIG. 167A-167C shows photographs, array map and description of FDA normal tissue array MNO1021 stained with the N+20/C-27 antibody 31A1 at 0.5 ug/mL. FIG. 167A shows photographs of the tissue micro array. FIG. 167B shows map of the array with abbreviated tissue descriptors. FIG. 167C detailed description of the tissue micro array with non-identifying donor data.

FIG. 168A-168X shows photographs of specific tissues from FDA normal tissue array MNO1021 stained with the N+20/C-27 antibody 31A1 at 0.5 ug/mL, magnified to 6× and 20×. FIG. 168A and FIG. 168E are adrenal gland. FIG. 168B and FIG. 168F are breast. FIG. 168C and FIG. 168G are fallopian tube. FIG. 168D and FIG. 168H are kidney. FIG. 168I and FIG. 168M are heart muscle. FIG. 168J and FIG. 168N are liver. FIG. 168K and FIG. 168O are lung. FIG. 168L and FIG. 168P are ureter. FIG. 168Q and FIG. 168U are eye. FIG. 168R and FIG. 168V are cerebral cortex. FIG. 168S and FIG. 168W are bone marrow. FIG. 168T and FIG. 168X are skeletal muscle.

FIG. 169A-169C shows photographs, array map and description of breast cancer tissue array BR1141 stained with the N+20/C-27 antibody 31A1 at 0.5 ug/mL. FIG. 169A shows photographs of the tissue micro array. FIG. 169B shows map of the array with abbreviated tissue descriptors. FIG. 169C detailed description of the tissue micro array with non-identifying donor data.

FIG. 170A-170F shows photographs of specific tissues from breast cancer tissue array BR1141 stained with the N+20/C-27 antibody 31A1 at 0.5 ug/mL, magnified to 6× and 20×. FIG. 170A and FIG. 170D are photographs of a Grade 2 invasive ductal carcinoma. FIG. 170B and FIG. 170E are photographs of a Grade 2 invasive ductal carcinoma. FIG. 170C and FIG. 170F are photographs of a Grade 2 invasive ductal carcinoma.

FIG. 171A-171C shows photographs, array map and description of pancreatic cancer tissue array PA1003 stained with the N+20/C-27 antibody 31A1 at 0.5 ug/mL. FIG. 171A shows photographs of the tissue micro array. FIG. 171B shows map of the array with abbreviated tissue descriptors. FIG. 171C detailed description of the tissue micro array with non-identifying donor data.

FIG. 172A-172F shows photographs of specific tissues from pancreatic cancer tissue array PA1003 stained with the N+20/C-27 antibody 31A1 at 0.5 ug/mL, magnified to 6× and 20×. FIG. 172A and FIG. 172D are photographs of a Grade 1 adenocarcinoma. FIG. 172B and FIG. 172E are photographs of a Grade 2 adenocarcinoma. FIG. 172C and FIG. 172F are photographs of a Grade 3 adenocarcinoma.

FIG. 173A-173C shows photographs, array map and description of FDA normal tissue array MNO1021 stained with the N+20/C-27 antibody 32C1 at 0.25 ug/mL. FIG. 173A shows photographs of the tissue micro array. FIG. 173B shows map of the array with abbreviated tissue descriptors. FIG. 173C detailed description of the tissue micro array with non-identifying donor data.

FIG. 174A-174X shows photographs of specific tissues from FDA normal tissue array MNO1021 stained with the N+20/C-27 antibody 32C1 at 0.25 ug/mL, magnified to 6× and 20×. FIG. 174A and FIG. 174E are adrenal gland. FIG. 174B and FIG. 174F are breast. FIG. 174C and FIG. 174G are fallopian tube. FIG. 174D and FIG. 174H are kidney. FIG. 174I and FIG. 174M are heart muscle. FIG. 174J and FIG. 174N are liver. FIG. 174K and FIG. 174O are lung. FIG. 174L and FIG. 174P are ureter. FIG. 174Q and FIG. 174U are eye. FIG. 174R and FIG. 174V are cerebral cortex. FIG. 174S and FIG. 174W are bone marrow. FIG. 174T and FIG. 174X are skeletal muscle.

FIG. 175A-175C shows photographs, array map and description of breast cancer tissue array BR1141 stained with the N+20/C-27 antibody 32C1 at 5.0 ug/mL. FIG. 175A shows photographs of the tissue micro array. FIG. 175B shows map of the array with abbreviated tissue descriptors. FIG. 175C detailed description of the tissue micro array with non-identifying donor data.

FIG. 176A-176F shows photographs of specific tissues from breast cancer tissue array BR1141 stained with the N+20/C-27 antibody 32C1 at 5.0 ug/mL, magnified to 6× and 20×. FIG. 176A and FIG. 176D are photographs of a Grade 2 invasive ductal carcinoma. FIG. 176B and FIG. 176E are photographs of a Grade 2 invasive ductal carcinoma. FIG. 176C and FIG. 176F are photographs of a Grade 2 invasive ductal carcinoma.

FIG. 177A-177C shows photographs, array map and description of esophageal cancer tissue array ES1001 stained with the N+20/C-27 antibody 32C1 at 1.0 ug/mL. FIG. 177A shows photographs of the tissue micro array. FIG. 177B shows map of the array with abbreviated tissue descriptors. FIG. 177C detailed description of the tissue micro array with non-identifying donor data.

FIG. 178A-178F shows photographs of specific tissues from esophageal cancer tissue array BC001113 stained with the N+20/C-27 antibody 32C1 at 1.0 ug/mL, magnified to 6× and 20×. FIG. 178A and FIG. 178D are photographs of a squamous cell carcinoma. FIG. 178B and FIG. 178E are photographs of an adenocarcinoma. FIG. 178C and FIG. 178F are photographs of a squamous cell carcinoma.

FIG. 179A-179C shows photographs, array map and description of FDA normal tissue array MNO1021 stained with the N+20/C-27 antibody 45C11 at 12.5 ug/mL. FIG. 179A shows photographs of the tissue micro array. FIG. 179B shows map of the array with abbreviated tissue descriptors. FIG. 179C detailed description of the tissue micro array with non-identifying donor data.

FIG. 180A-180X shows photographs of specific tissues from FDA normal tissue array MNO1021 stained with the N+20/C-27 antibody 45C11 at 12.5 ug/mL, magnified to 6× and 20×. FIG. 180A and FIG. 180E are adrenal gland. FIG. 180B and FIG. 180F are breast. FIG. 180C and FIG. 180G are fallopian tube. FIG. 180D and FIG. 180H are kidney. FIG. 180I and FIG. 180M are heart muscle. FIG. 180J and FIG. 180N are liver. FIG. 180K and FIG. 180O are lung. FIG. 180L and FIG. 180P are ureter. FIG. 180Q and FIG. 180U are eye. FIG. 180R and FIG. 180V are cerebral cortex. FIG. 180S and FIG. 180W are bone marrow. FIG. 180T and FIG. 180X are skeletal muscle.

FIG. 181A-181C shows photographs, array map and description of breast cancer tissue array BR1007 stained with the N+20/C-27 antibody 45C11 at 10.0 ug/mL. FIG. 181A shows photographs of the tissue micro array. FIG. 181B shows map of the array with abbreviated tissue descriptors. FIG. 181C detailed description of the tissue micro array with non-identifying donor data.

FIG. 182A-182F shows photographs of specific tissues from breast cancer tissue array BR1007 stained with the N+20/C-27 antibody 45C11 at 10.0 ug/mL, magnified to 6× and 20×. FIG. 182A and FIG. 182D are photographs of a Grade 2 invasive ductal carcinoma with positive lymph nodes. FIG. 182B and FIG. 182E are photographs of a Grade 2 invasive ductal carcinoma. FIG. 182C and FIG. 182F are photographs of a Grade 2 invasive ductal carcinoma.

FIG. 183A-183C shows photographs, array map and description of pancreatic cancer tissue array PA805c stained with the N+20/C-27 antibody 45C11 at 12.5 ug/mL. FIG. 183A shows photographs of the tissue micro array. FIG. 183B shows map of the array with abbreviated tissue descriptors. FIG. 183C detailed description of the tissue micro array with non-identifying donor data.

FIG. 184A-184F shows photographs of specific tissues from pancreatic cancer tissue array PA805c stained with the N+20/C-27 antibody 45C11 at 12.5 ug/mL, magnified to 6× and 20×. FIG. 184A and FIG. 184D are photographs of a Grade 2 papillary adenocarcinoma. FIG. 184B and FIG. 184E are photographs of a Grade 2-3 ductal carcinoma. FIG. 184C and FIG. 184F are photographs of a Grade 3 invasive adenocarcinoma.

FIG. 185A-185C shows photographs, array map and description of FDA normal tissue array MNO1021 stained with the N+9/C-9 antibody 3C5 at 10.0 ug/mL. FIG. 185A shows photographs of the tissue micro array. FIG. 185B shows map of the array with abbreviated tissue descriptors. FIG. 185C detailed description of the tissue micro array with non-identifying donor data.

FIG. 186A-186X shows photographs of specific tissues from FDA normal tissue array MNO1021 stained with the N+9/C-9 antibody 3C5 at 10.0 ug/mL, magnified to 6× and 20×. FIG. 186A and FIG. 186E are adrenal gland. FIG. 186B and FIG. 186F are breast. FIG. 186C and FIG. 186G are fallopian tube. FIG. 186D and FIG. 186H are kidney. FIG. 186I and FIG. 186M are heart muscle. FIG. 186J and FIG. 186N are liver. FIG. 186K and FIG. 186O are lung. FIG. 186L and FIG. 186P are ureter. FIG. 186Q and FIG. 186U are eye. FIG. 186R and FIG. 186V are cerebral cortex. FIG. 186S and FIG. 186W are bone marrow. FIG. 186T and FIG. 186X are skeletal muscle.

FIG. 187A-187C shows photographs, array map and description of pancreatic cancer tissue array PA1003 stained with the N+9/C-9 antibody 3C5 at 10.0 ug/mL. FIG. 187A shows photographs of the tissue micro array. FIG. 187B shows map of the array with abbreviated tissue descriptors. FIG. 187C detailed description of the tissue micro array with non-identifying donor data.

FIG. 188A-188F shows photographs of specific tissues from pancreatic cancer tissue array PA1003 stained with the N+9/C-9 antibody 3C5 at 10.0 ug/mL, magnified to 6× and 20×. FIG. 188A and FIG. 188D are photographs of a Grade 2 adenocarcinoma. FIG. 188B and FIG. 188E are photographs of a Grade 2 adenocarcinoma. FIG. 188C and FIG. 188F are photographs of a Grade 2-3 adenocarcinoma with lymph node involvement.

FIG. 189A-189C shows photographs, array map and description of FDA normal tissue array MNO1021 stained with the N+9/C-9 antibody 8A9 at 15.0 ug/mL. FIG. 189A shows photographs of the tissue micro array. FIG. 189B shows map of the array with abbreviated tissue descriptors. FIG. 189C detailed description of the tissue micro array with non-identifying donor data.

FIG. 190A-190X shows photographs of specific tissues from FDA normal tissue array MNO1021 stained with the N+9/C-9 antibody 8A9 at 15.0 ug/mL, magnified to 6× and 20×. FIG. 190A and FIG. 190E are adrenal gland. FIG. 190B and FIG. 190F are breast. FIG. 190C and FIG. 190G are fallopian tube. FIG. 190D and FIG. 190H are kidney. FIG. 190I and FIG. 190M are heart muscle. FIG. 190J and FIG. 190N are liver. FIG. 190K and FIG. 190O are lung. FIG. 190L and FIG. 190P are ureter. FIG. 190Q and FIG. 190U are eye. FIG. 190R and FIG. 190V are cerebral cortex. FIG. 190S and FIG. 190W are bone marrow. FIG. 190T and FIG. 190X are skeletal muscle.

FIG. 191A-191C shows photographs, array map and description of pancreatic cancer tissue array PA1003 stained with the N+9/C-9 antibody 8A9 at 15.0 ug/mL. FIG. 191A shows photographs of the tissue micro array. FIG. 191B shows map of the array with abbreviated tissue descriptors. FIG. 191C detailed description of the tissue micro array with non-identifying donor data.

FIG. 192A-192F shows photographs of specific tissues from pancreatic cancer tissue array PA1003 stained with the N+9/C-9 antibody 8A9 at 15.0 ug/mL, magnified to 6× and 20×. FIG. 192A and FIG. 192D are photographs of a Grade 2 adenocarcinoma. FIG. 192B and FIG. 192E are photographs of a Grade 2 adenocarcinoma. FIG. 192C and FIG. 192F are photographs of a Grade 2 adenocarcinoma.

FIG. 193A-193C shows photographs, array map and description of FDA normal tissue array MNO1021 stained with the N+9/C-9 antibody 17H6 at 30.0 ug/mL. FIG. 193A shows photographs of the tissue micro array. FIG. 193B shows map of the array with abbreviated tissue descriptors. FIG. 193C detailed description of the tissue micro array with non-identifying donor data.

FIG. 194A-194X shows photographs of specific tissues from FDA normal tissue array MNO1021 stained with the N+9/C-9 antibody 17H6 at 30.0 ug/mL, magnified to 6× and 20×. FIG. 194A and FIG. 194E are adrenal gland. FIG. 194B and FIG. 194F are breast. FIG. 194C and FIG. 194G are fallopian tube. FIG. 194D and FIG. 194H are kidney. FIG. 194I and FIG. 194M are heart muscle. FIG. 194J and FIG. 194N are liver. FIG. 194K and FIG. 194O are lung. FIG. 194L and FIG. 194P are ureter. FIG. 194Q and FIG. 194U are eye. FIG. 194R and FIG. 194V are cerebral cortex. FIG. 194S and FIG. 194W are bone marrow. FIG. 194T and FIG. 194X are skeletal muscle.

FIG. 195A-195C shows photographs, array map and description of pancreatic cancer tissue array PA805c stained with the N+9/C-9 antibody 17H6 at 30.0 ug/mL. FIG. 195A shows photographs of the tissue micro array. FIG. 195B shows map of the array with abbreviated tissue descriptors. FIG. 195C detailed description of the tissue micro array with non-identifying donor data.

FIG. 196A-196F shows photographs of specific tissues from pancreatic cancer tissue array PA805c stained with the N+9/C-9 antibody 17H6 at 30.0 ug/mL, magnified to 6× and 20×. FIG. 196A and FIG. 196D are photographs of a Grade 2 papillary adenocarcinoma. FIG. 196B and FIG. 196E are photographs of a Grade 2-3 ductal carcinoma with lymph node involvement. FIG. 196C and FIG. 196F are photographs of a Grade 3 invasive adenocarcinoma.

FIG. 197A-197C shows photographs, array map and description of FDA normal tissue array MNO1021 stained with the N+9/C-9 antibody 39H5 at 5.0 ug/mL. FIG. 197A shows photographs of the tissue micro array. FIG. 197B shows map of the array with abbreviated tissue descriptors. FIG. 197C detailed description of the tissue micro array with non-identifying donor data.

FIG. 198A-198X shows photographs of specific tissues from FDA normal tissue array MNO1021 stained with the N+9/C-9 antibody 39H5 at 5.0 ug/mL, magnified to 6× and 20×. FIG. 198A and FIG. 198E are adrenal gland. FIG. 198B and FIG. 198F are breast. FIG. 198C and FIG. 198G are fallopian tube. FIG. 198D and FIG. 198H are kidney. FIG. 198I and FIG. 198M are heart muscle. FIG. 198J and FIG. 198N are liver. FIG. 198K and FIG. 198O are lung. FIG. 198L and FIG. 198P are ureter. FIG. 198Q and FIG. 198U are eye. FIG. 198R and FIG. 198V are cerebral cortex. FIG. 198S and FIG. 198W are bone marrow. FIG. 198T and FIG. 198X are skeletal muscle.

FIG. 199A-199C shows photographs, array map and description of pancreatic cancer tissue array PA1003 stained with the N+9/C-9 antibody 39H5 at 5.0 ug/mL. FIG. 199A shows photographs of the tissue micro array. FIG. 199B shows map of the array with abbreviated tissue descriptors. FIG. 199C detailed description of the tissue micro array with non-identifying donor data.

FIG. 200A-200F shows photographs of specific tissues from pancreatic cancer tissue array PA1003 stained with the N+9/C-9 antibody 39H5 at 5.0 ug/mL, magnified to 6× and 20×. FIG. 200A and FIG. 200D are photographs of a Grade 2 adenocarcinoma. FIG. 200B and FIG. 200E are photographs of a Grade 2 adenocarcinoma. FIG. 200C and FIG. 200F are photographs of a Grade 2 adenocarcinoma.

FIG. 201A-201C show graphs of ELISA assays to determine the binding of another set of antibodies generated by immunizing animals with the PSMGFR peptide. FIG. 201A shows binding to the PSMGFR peptide. FIG. 201B shows binding to the N-10 peptide. FIG. 201C shows binding to the C-10 peptide. As can be seen, none of the antibodies bound to the C-10 peptide. F3, B12, B2, B7, B9, 8C7F3 and H11 all bound to the PSMGFR peptide and to the N-10 peptide.

FIG. 202A-202C shows photographs of pancreatic cancer tissue array PA1003 that has been stained with monoclonal antibody 1E4, monoclonal antibody 18B4 or the polyclonal anti-PSMGFR antibody SDIX. 18B4 binds to the GTINVHDVET epitope at the most N-terminal portion of the PSMGFR peptide, while the 1E4 antibody binds to the QFNQYKTEA epitope that is immediately adjacent and C-terminal to the 18B4 epitope.

FIG. 203A-203F shows magnified images of the tissue specimen at position A2 of the pancreatic cancer array PA1003. FIG. 203A and FIG. 203B show the specimen stained with antibody 1E4. FIG. 203C and FIG. 203D show the specimen stained with antibody 18B4. FIG. 203E and FIG. 203F show the specimen stained with polyclonal antibody SDIX.

FIG. 204A-204D shows magnified images of the tissue specimen at position D4 of the pancreatic array PA1003. FIG. 204A and FIG. 204B show the specimen stained with antibody 18B4. FIG. 204C and FIG. 204D show the specimen stained with polyclonal antibody SDIX.

FIG. 205A-205D shows magnified images of the tissue specimen at position E1 of the pancreatic cancer array PA1003. FIG. 205A and FIG. 205B show the specimen stained with antibody 18B4. FIG. 205C and FIG. 205D show the specimen stained with polyclonal antibody SDIX.

FIG. 206A-206D shows magnified images of the tissue specimen at position C3 of the pancreatic cancer array PA1003. FIG. 206A and FIG. 206B show the specimen stained with antibody 1E4. FIG. 206C and FIG. 206D show the specimen stained with polyclonal antibody SDIX.

FIG. 207A-207D shows magnified images of the tissue specimen at position D1 of the pancreatic cancer array PA1003. FIG. 207A and FIG. 207B show the specimen stained with antibody 1E4. FIG. 207C and FIG. 207D show the specimen stained with polyclonal antibody SDIX.

FIG. 208A-208C shows photographs of the pancreatic cancer array PA1003. FIG. 208A shows the specimen stained with polyclonal antibody SDIX. FIG. 208B shows the specimen stained with antibody 20A10. FIG. 208C shows the specimen stained with antibody 29H1.

FIG. 209A-209D shows photographs of the esophageal cancer array ES1001 stained with various antibodies. FIG. 209A shows the array stained with polyclonal antibody SDIX. FIG. 209B shows the array stained with antibody 20A10. FIG. 209C shows the array stained with antibody 29H1. FIG. 209D shows the array stained with antibody 31A1.

FIG. 210A-210C shows photographs of the pancreatic cancer array PA1003 stained with various antibodies. FIG. 210A shows the array stained with polyclonal antibody SDIX. FIG. 210B shows the array stained with antibody 20A10. FIG. 210C shows the array stained with antibody 29H1.

FIG. 211A-211C show graphs of an ELISA experiment measuring the amount of IL-18 secreted into the condition media of MUC1* positive cancer cells co-cultured with huMNC2-CAR44 T cells wherein the cells also bear an NFAT inducible IL-18. FIG. 211A shows the graph of IL-18 secreted into the supernatant of T47D breast cancer cells co-cultured with untransduced human T cells. FIG. 211B shows the graph of IL-18 secreted into the supernatant of T47D breast cancer cells co-cultured with huMNC2-CAR44 T cells that also bore an NFAT inducible IL-18 gene inserted into a portion of the Foxp3 enhancer. FIG. 211C shows the graph of IL-18 secreted into the supernatant of T47D breast cancer cells co-cultured with huMNC2-CAR44 T cells that also bore an NFAT inducible IL-18 gene inserted into a portion of the IL-2 enhancer.

FIG. 212A-212X shows photographs of T47D breast cancer cells (red) doped with varying percentages of T47D cells engineered to express more MUC1* (green). The target cancer cells have been co-cultured with huMNC2-CAR44 T cells with NFAT inducible IL-18 wherein the IL-18 gene has been inserted into either the Foxp3 enhancer/promoter or the IL-2 enhancer/promoter. FIGS. 212A-212C, 212I-212K, and 212Q-212S show the cancer cells co-cultured with untranduced T cells. FIGS. 212D-212F, 212L-212N, and 212T-212V show the cancer cells co-cultured with hiMNC2-CAR44 T cells with the NFAT inducible IL-18 gene inserted into the Foxp3 enhancer/promoter. FIGS. 212G-212H, 2120-212P, and 212W-212X show the cancer cells co-cultured with hiMNC2-CAR44 T cells with the NFAT inducible IL-18 gene inserted into the IL-2 enhancer/promoter.

FIG. 213A-213B shows graphs of ELISA experiments in which levels of IL-18 secreted into the conditioned media are measured for huMNC1-CAR44 T cells with NFAT inducible IL-18 gene, inserted into the Foxp3 enhancer or promoter, co-cultured with either MUC1* positive cancer cells or MUC1 negative non-cancerous cells. FIG. 213A shows IL-18 secretion from huMNC2-CAR44 T cells with NFAT inducible IL-18 in co-culture with T47D breast cancer cells where the population has been doped with 5%, 10% or 30% T47D cells that had been transfected with even more MUC1*. FIG. 213B shows IL-18 secretion from huMNC2-CAR44 T cells with NFAT inducible IL-18 in co-culture with non-cancerous, MUC1 negative HEK293 cells where the cell population has been doped with 5%, 10% or 30% T47D cells that had been transfected with more MUC1*.

FIG. 214A-214X shows photographs of T47D breast cancer cells (red) or non-cancerous HEK293 cells (also red), where both cell types have been doped with varying percentages of T47D cells engineered to express more MUC1* (green). These target cancer cells have been co-cultured with huMNC2-CAR44 T cells with NFAT inducible IL-18 wherein the IL-18 gene has been inserted into the Foxp3 enhancer/promoter. FIG. 214A-214F shows either T47D cells or HEK293 cells that have not been doped with T47D cells engineered to express high MUC1* density. FIG. 214G-214L shows either T47D cells or HEK293 cells that have been doped with 5% T47D cells engineered to express high MUC1* density. FIG. 214M-214R shows either T47D cells or HEK293 cells that have been doped with 10% T47D cells engineered to express high MUC1* density. FIG. 2145-214X shows either T47D cells or HEK293 cells that have been doped with 30% T47D cells engineered to express high MUC1* density. FIGS. 214A-B, G-H, M-N, and S-T show T47D breast cancer cells. FIGS. 214C-F, I-L, O-R, and U-X show HEK293 cells. As can be seen in the figures, the induced secretion of IL-18 resulted in low MUC1* density T47D cells being killed but did not induce non-specific killing of the MUC1* negative HEK293 cells.

FIG. 215A-215C shows the consensus sequences of the heavy chain CDRs wherein the consensus sequences were generated for each group of antibodies that bound to the same epitope in the PSMGFR and N-terminally extended PSMGFR peptide. FIG. 215A shows consensus sequences for heavy chain CDR1. FIG. 215B shows consensus sequences for heavy chain CDR2. FIG. 215C shows consensus sequences for heavy chain CDR3.

FIG. 216A-216C shows the consensus sequences of the light chain CDRs wherein the consensus sequences were generated for each group of antibodies that bound to the same epitope in the PSMGFR and N-terminally extended PSMGFR peptide. FIG. 216A shows consensus sequences for light chain CDR1. FIG. 216B shows consensus sequences for light chain CDR2. FIG. 216C shows consensus sequences for light chain CDR3.

FIG. 217 shows alternative formats for bispecific antibodies and other bispecific immunotherapeutics subdivided into five major classes: BsIgG, appended IgG, BsAb fragments, bispecific fusion proteins and BsAb conjugates. Heavy chains are shown in dark blue, dark pink and dark green and corresponding light chains are in lighter shades of the same colors. Connecting peptide linkers are shown by thin black lines and engineered disulfide bonds by thin green lines. Approximate molecular weights are shown assuming ˜12.5 kDa per immunoglobulin domain. BsAb formats that have advanced into clinical testing are highlighted (*). For interpretation of the references to color in this figure description, the reader is referred to the web version of the article, Spiess et al. Molecular Immunology 67, 95-106 (2015), the contents of which are incorporated by reference in their entirety, in particular with respect to the description of FIG. 1 of Spiess et al., as well as other descriptions of various methods of making and using bispecific antibody fragments).

FIG. 218 shows graphs of tumor volume measured by an IVIS instrument wherein the tumor cells have been genetically modified to express Luciferase. The substrate Luciferin was injected 10 minutes before the photo emissions were measured in the sedated animal. On Day 1 of the experiment, animals were injected sub-cutaneously with 250,000 human breast tumor cells. Tumors were made heterogeneous, comprised of two different tumor cell types. A first tumor cell population was T47D-wt, a breast cancer cell line that expresses both full-length MUC1 and the growth factor receptor form MUC1*, which we engineered to express mCherry fluorescence. The second tumor cell population was the same T47D breast cancer cells, except that they had been stably transduced to express even more MUC1* and GFP fluorescence, referred to here as T47D-MUC1*. In this experiment, animals were implanted with T47D-wt plus T47D-MUC1*, wherein the population of T47D-MUC1* made up 30%, 15% or 7.5% of the tumor population. Animals were then administered a one-time injection of either PBS, huMNC2-41BB-3z CAR T cells (4-1BB), huMNC2-CD28-3z CAR T cells (CD28) or huMNC2-CD28-1XX CAR T cells (CD28-1XX or 1XX). The CAR T cells were injected into the tail vein at an Effector to Target ratio (E:T) of 10:1, 5:1, or 1:1.

FIG. 219A-219B shows IVIS photographs and graphs of IVIS tumor volume measurement. FIG. 219A shows photographs of the mice that had been implanted with tumors in which 30% of the cancer cell population was T47D-MUC1*, referred to here as high antigen expressing cells. The various CAR T cells were administered at a CAR T to tumor cell ratio of 10:1 wherein 250,000 tumor cells were implanted and the animals were injected 5 days later with 2,500,000 CAR T cells. FIG. 219B shows a graph of the tumor volume by IVIS measurement by day. As can be seen, animals injected with the huMNC2-CD28-1XX had much smaller tumors than the animals treated huMNC2-4-1BB-3z or huMNC2-CD28-3z, which is the same CAR T except without the 1XX mutations in the CD3-zeta domain.

FIG. 220A-220T shows the IVIS graphs for 30% tumors treated at a CAR T to Tumor ratio of 10:1. Here graphs are shown for each individual animal rather than the average of the treatment group.

FIG. 221A-221B shows IVIS photographs and graphs of IVIS tumor volume measurement. FIG. 221A shows photographs of the mice that had been implanted with tumors in which 30% of the cancer cell population was T47D-MUC1*, referred to here as high antigen expressing cells. The various CAR T cells were administered at a CAR T to tumor cell ratio of 1:1 wherein 250,000 tumor cells were implanted and the animals were injected 5 days later with 250,000 CAR T cells. FIG. 221B shows a graph of the tumor volume by IVIS measurement by day. As can be seen, animals injected with the huMNC2-CD28-1XX had much smaller tumors than the animals treated huMNC2-4-1BB-3z or huMNC2-CD28-3z, which is the same CAR T except without the 1XX mutations in the CD3-zeta domain. However, with the lower dose of CAR T cells, even tumors in the huMNC2-CD28-1XX treated group begin to grow again.

FIG. 222A-222T shows the IVIS graphs for 30% tumors treated at a CAR T to Tumor ratio of 1:1. Here graphs are shown for each individual animal rather than the average of the treatment group.

FIG. 223A-223B shows IVIS photographs and graphs of IVIS tumor volume measurement. FIG. 223A shows photographs of the mice that had been implanted with tumors in which 7.5% of the cancer cell population was T47D-MUC1*, referred to here as high antigen expressing cells. The various CAR T cells were administered at a CAR T to tumor cell ratio of 10:1 wherein 250,000 tumor cells were implanted and the animals were injected 5 days later with 2,500,000 CAR T cells. FIG. 223B shows a graph of the tumor volume by IVIS measurement by day. As can be seen, animals injected with the huMNC2-CD28-1XX had much smaller tumors than the animals treated huMNC2-4-1BB-3z or huMNC2-CD28-3z, which is the same CAR T except without the 1XX mutations in the CD3-zeta domain. However, even tumors in the huMNC2-CD28-1XX treated group begin to grow again, which is consistent with the idea that a small percentage of high antigen expressing tumor cells impedes the killing of the rest of the tumor.

FIG. 224A-224T shows the IVIS graphs for 7.5% tumors treated at a CAR T to Tumor ratio of 10:1. Here graphs are shown for each individual animal rather than the average of the treatment group.

FIG. 225A-225B shows IVIS photographs and graphs of IVIS tumor volume measurement. FIG. 225A shows photographs of the mice that had been implanted with tumors in which 7.5% of the cancer cell population was T47D-MUC1*, referred to here as high antigen expressing cells. The various CAR T cells were administered at a CAR T to tumor cell ratio of 1:1 wherein 250,000 tumor cells were implanted and the animals were injected 5 days later with 250,000 CAR T cells. FIG. 225B shows a graph of the tumor volume by IVIS measurement by day. As can be seen, animals injected with the huMNC2-CD28-1XX had smaller tumors than the animals treated huMNC2-4-1BB-3z or huMNC2-CD28-3z, which is the same CAR T except without the 1XX mutations in the CD3-zeta domain. However, at low antigen density combined with very low dose of CAR T cells, even tumors in the huMNC2-CD28-1XX treated group begin to grow again.

FIG. 226A-226T shows the IVIS graphs for 7.5% tumors treated at a CAR T to Tumor ratio of 1:1. Here graphs are shown for each individual animal rather than the average of the treatment group.

FIG. 227 shows the tabulation of CD3 positive human T cells that were harvested from the spleens of the test animals post sacrifice. In this table, cells isolated from mice implanted with tumors comprised of 30% T47D-MUC1* and treated with CAR T cells at a 10:1 ratio. As can be seen, the huMNC2-CD28-1XX treated mice, that had smaller tumors, have the greater numbers of CAR T cells and CD8 positive killer T cells. TIM3, LAG3 and PD-1 are molecular markers of T cell exhaustion. The table shows that the huMNC2-CD28-1XX CAR T cells harvested from the animals express lower levels of exhaustion markers, consistent with the idea that the 1XX mutations in CD3-zeta increase CAR T cell persistence in vivo.

FIG. 228 shows the tabulation of CD3 positive human T cells that were harvested from the spleens of the test animals post sacrifice. In this table, cells isolated from mice implanted with tumors comprised of 30% T47D-MUC1* and treated with CAR T cells at a 1:1 ratio. As can be seen, the huMNC2-CD28-1XX treated mice, that had smaller tumors, have the greater numbers of CAR T cells and CD8 positive killer T cells. TIM3, LAG3 and PD-1 are molecular markers of T cell exhaustion. The table shows that the huMNC2-CD28-1XX CAR T cells harvested from the animals express lower levels of exhaustion markers, consistent with the idea that the 1XX mutations in CD3-zeta increase CAR T cell persistence in vivo.

FIG. 229 shows the tabulation of CD3 positive human T cells that were harvested from the blood of the test animals post sacrifice. In this table, cells isolated from mice implanted with tumors comprised of 30% T47D-MUC1* and treated with CAR T cells at a 1:1 ratio. As can be seen, the huMNC2-CD28-1XX treated mice, that had smaller tumors, have the greater numbers of CAR T cells and CD8 positive killer T cells and have lower levels of T cell exhaustion markers.

FIG. 230 shows the tabulation of CD3 positive human T cells that were harvested from the spleens of the test animals post sacrifice. In this table, cells isolated from mice implanted with tumors comprised of 7.5% T47D-MUC1* and treated with CAR T cells at a 10:1 ratio. As can be seen, the huMNC2-CD28-1XX treated mice, that had smaller tumors, have the greater numbers of CAR T cells and CD8 positive killer T cells. TIM3, LAG3 and PD-1 are molecular markers of T cell exhaustion. The table shows that the huMNC2-CD28-1XX CAR T cells harvested from the animals express lower levels of exhaustion markers, consistent with the idea that the 1XX mutations in CD3-zeta increase CAR T cell persistence in vivo.

FIG. 231 shows the tabulation of CD3 positive human T cells that were harvested from the blood of the test animals post sacrifice. In this table, cells isolated from mice implanted with tumors comprised of 7.5% T47D-MUC1* and treated with CAR T cells at a 10:1 ratio. As can be seen, the huMNC2-CD28-1XX treated mice, that had smaller tumors, have the greater numbers of CAR T cells and CD8 positive killer T cells. TIM3, LAG3 and PD-1 are molecular markers of T cell exhaustion. The table shows that the huMNC2-CD28-1XX CAR T cells harvested from the animals express lower levels of exhaustion markers, consistent with the idea that the 1XX mutations in CD3-zeta increase CAR T cell persistence in vivo.

FIG. 232 shows the tabulation of CD3 positive human T cells that were harvested from the spleens of the test animals post sacrifice. In this table, cells isolated from mice implanted with tumors comprised of 7.5% T47D-MUC1* and treated with CAR T cells at a 1:1 ratio. As can be seen, the huMNC2-CD28-1XX treated mice, that had smaller tumors, have the greater numbers of CAR T cells and CD8 positive killer T cells. TIM3, LAG3 and PD-1 are molecular markers of T cell exhaustion. The table shows that the huMNC2-CD28-1XX CAR T cells harvested from the animals express lower levels of exhaustion markers, consistent with the idea that the 1XX mutations in CD3-zeta increase CAR T cell persistence in vivo.

FIG. 233 shows the tabulation of CD3 positive human T cells that were harvested from the blood of the test animals post sacrifice. In this table, cells isolated from mice implanted with tumors comprised of 7.5% T47D-MUC1* and treated with CAR T cells at a 1:1 ratio. As can be seen, the huMNC2-CD28-1XX treated mice, that had smaller tumors, have the greater numbers of CAR T cells and CD8 positive killer T cells. TIM3, LAG3 and PD-1 are molecular markers of T cell exhaustion. The table shows that the huMNC2-CD28-1XX CAR T cells harvested from the animals express lower levels of exhaustion markers, consistent with the idea that the 1XX mutations in CD3-zeta increase CAR T cell persistence in vivo.

FIG. 234A-234U shows photographs of the tumors excised from test animals and shows their weight in grams. Tumors were excised from animals implanted with tumors made up of 30% T47D-MUC1* high antigen density cells and 70% T47D-wt low antigen density cells. Animals were treated with CAR T cells at an effector to target ratio of 10:1.

FIG. 235A-235N2 shows magnified photographs of dissociated tumors excised from animals implanted with tumors made up of 30% T47D-MUC1* high antigen density cells and 70% T47D-wt low antigen density cells. Animals were treated with CAR T cells at an effector to target ratio of 10:1. Shown are overlays of bright field images and fluorescent images, wherein the red fluorescence, mCherry, shows the low antigen density cells and the green fluorescence, GFP, shows the low antigen density cells. FIG. 235A-235J show tumor cells excised from the control animals treated only with PBS. FIG. 235K-235T show tumor cells excised from the animals treated with huMNC2-41BB-3z CAR T cells. FIG. 235U-235D2 show tumor cells excised from the animals treated with huMNC2-CD28-1XX CAR T cells. FIG. 235E2-235N2 show tumor cells excised from the animals treated with huMNC2-CD28-3z CAR T cells.

FIG. 236A-236U shows photographs of the tumors excised from test animals and shows their weight in grams. Tumors were excised from animals implanted with tumors made up of 30% T47D-MUC1* high antigen density cells and 70% T47D-wt low antigen density cells. Animals were treated with CAR T cells at an effector to target ratio of 1:1. FIG. 236A-236E show tumors excised from animals mock treated with PBS. FIG. 236F-236J show tumors excised from animals treated with huMNC2-41BB-3z. FIG. 236K-2360 show tumors excised from animals treated with huMNC2-CD28-1XX. FIG. 236P-236T show tumors excised from animals treated with huMNC2-CD28-3z. FIG. 236U shows a bar graph of the weights of tumors excised from the test animals.

FIG. 237A-237D2 shows magnified photographs of dissociated tumors excised from animals implanted with tumors made up of 30% T47D-MUC1* high antigen density cells and 70% T47D-wt low antigen density cells. Animals were treated with CAR T cells at an effector to target ratio of 1:1. Shown are overlays of bright field images and fluorescent images, wherein the red fluorescence, mCherry, shows the low antigen density cells and the green fluorescence, GFP, shows the low antigen density cells. FIG. 237A-237J show tumor cells excised from the control animals treated only with PBS. FIG. 237K-237T show tumor cells excised from the animals treated with huMNC2-41BB-3z CAR T cells. FIG. 237U-237D2 show tumor cells excised from the animals treated with huMNC2-CD28-1XX CAR T cells. FIG. 237E2-237N2 show tumor cells excised from the animals treated with huMNC2-CD28-3z CAR T cells.

FIG. 238A-238T shows photographs of the tumors excised from test animals and shows their weight in grams. Tumors were excised from animals implanted with tumors made up of 7.5% T47D-MUC1* high antigen density cells and 92.5% T47D-wt low antigen density cells. Animals were treated with CAR T cells at an effector to target ratio of 10:1.

FIG. 239A-239M2 shows magnified photographs of dissociated tumors excised from animals implanted with tumors made up of 7.5% T47D-MUC1* high antigen density cells and 92.5% T47D-wt low antigen density cells. Animals were treated with CAR T cells at an effector to target ratio of 10:1. Shown are overlays of bright field images and fluorescent images, wherein the red fluorescence, mCherry, shows the low antigen density cells and the green fluorescence, GFP, shows the low antigen density cells. FIG. 239A-239J show tumor cells excised from animals mock treated with PBS. FIG. 239K-239T show tumor cells excised from the animals treated with huMNC2-41BB-3z CAR T cells. FIG. 239U-239C2 show tumor cells excised from the animals treated with huMNC2-CD28-1XX CAR T cells. FIG. 239D2-239M2 show tumor cells excised from the animals treated with huMNC2-CD28-3z CAR T cells.

FIG. 240A-2400 shows photographs of the tumors excised from test animals and shows their weight in grams. Tumors were excised from animals implanted with tumors made up of 7.5% T47D-MUC1* high antigen density cells and 92.5% T47D-wt low antigen density cells. Animals were treated with CAR T cells at an effector to target ratio of 1:1.

FIG. 241A-241D2 shows magnified photographs of dissociated tumors excised from animals implanted with tumors made up of 7.5% T47D-MUC1* high antigen density cells and 92.5% T47D-wt low antigen density cells. Animals were treated with CAR T cells at an effector to target ratio of 1:1. Shown are overlays of bright field images and fluorescent images, wherein the red fluorescence, mCherry, shows the low antigen density cells and the green fluorescence, GFP, shows the low antigen density cells. FIG. 241A-241J show tumor cells excised from control animals treated only with PBS. FIG. 241K-241T show tumor cells excised from the animals treated with huMNC2-41BB-3z CAR T cells. FIG. 241U-241D2 show tumor cells excised from the animals treated with huMNC2-CD28-1XX CAR T cells. FIG. 241E2-241N2 show tumor cells excised from the animals treated with huMNC2-CD28-3z CAR T cells.

FIG. 242A-242R shows photographs of live animals, where IVIS measures tumor volume, mCherry detects the low antigen cells within the tumor and GFP detects the high antigen cells within the tumor. Post sacrifice photographs are shown of the excised tumors and a graph of tumor weights. A graph of the GFP positive high antigen tumor cells is also shown. Both the live GFP photographs and the graph of FACS measurement of high antigen cells show that huMNC2-CD28-1XX has killed all the high antigen cells and most of the low antigen cells even with the lowest levels of high antigen cells in the tumor and at very low dose of CAR T cells.

FIG. 243A-243F shows photographs taken at two different timepoints. Here, IVIS photographs measure tumor volume, mCherry fluorescent photographs measure low antigen cells and GFP fluorescent photographs measure high antigen cells. In this case, the animals were implanted with tumors made up of 30% high antigen density cells (GFP+) and 70% low antigen density cells (mCherry+). Animals had been given a single dose of CAR T cells at a 10:1 or a 1:1 CAR T to tumor cell ratio.

FIG. 244 shows graphs of IVIS tumor volume measurements over time. Arrows indicate timepoints when fluorescent photographs, mCherry and GFP, of live animals were taken. In this case, the animals were implanted with tumors made up of 30% high antigen density cells (GFP+) and 70% low antigen density cells (mCherry+). Animals had been given a single dose of CAR T cells at a 10:1 or a 1:1 CAR T to tumor cell ratio.

FIG. 245 shows graphs of IVIS measurements of tumor volume, mCherry measurements of the growth rate of low antigen cells and GFP measurements of the growth rate of high antigen cells, between two timepoints. As can be seen, at high CAR T dose, there is no increase in the number of high antigen density cells (GFP) over time in mice treated with either huMNC2-41BB-3z or huMNC2-CD28-1XX. However, at low CAR T dose there is some of the mice treated with huMNC2-41BB-3z do show an increase in the growth of high antigen density cells, whereas mice treated with huMNC2-CD28-1XX do not. More importantly, at high or low CAR T dose, mice treated with huMNC2-41BB-3z show increases in the growth of low antigen density cells, whereas in mice treated with huMNC2-CD28-1XX the growth of low antigen density cells is more controlled.

FIG. 246A-246B shows IVIS photographs and graphs of IVIS tumor volume measurement. FIG. 246A shows photographs of the mice that had been implanted with tumors in which 15% of the cancer cell population was T47D-MUC1*, referred to here as high antigen expressing cells. The various CAR T cells were administered at a CAR T to tumor cell ratio of 10:1 wherein 250,000 tumor cells were implanted and the animals were injected 5 days later with 2,500,000 CAR T cells. FIG. 246B shows a graph of the tumor volume by IVIS measurement by day. In this experiment, on Day 52, animals outlined in red were implanted with 250,000 more 100% high antigen density tumor cells. The animals outlined in green received an additional dose of 2,500,000 CAR T cells.

FIG. 247A-247T shows graphs of the IVIS measured growth of the tumors in each individual animal. The red arrows indicate injection of more tumor cells and the green arrows indicate injection of 2,500,000 additional CAR T cells. As can be seen, the injection of additional tumor cells does not increase the tumor growth in animals treated with huMNC2-CD28-1XX CAR T cells. However, the injection of additional tumor cells does increase tumor growth in animals treated with huMNC2-CD28-3z or huMNC2-41BB-3z CAR T cells. This result is consistent with CAR T cells with 1XX mutations in CD3z prolong CAR T cell persistence in vivo. It can also be seen that the injection of additional CAR T cells suppressed tumor cells in some of the animals in all groups. This is consistent with the idea that tumor recurrence was not due to tumor escape because the fresh CAR T cells still recognized the tumor cells and killed them.

FIG. 248A-247D shows a cartoon of the experimental strategy wherein animals were implanted with heterologous tumors comprised of two cell types that carry two different florescent labels. Animals were implanted with both T47D breast cancer cells that bear mCherry and fluoresce red and also implanted with T47D cells that have been engineered to express even more MUC1* and bear GFP making them fluoresce green. FIG. 248A shows cartoon of animals implanted with tumors in which 30% of the tumors express high levels of MUC1* and those tumor cells fluoresce green. FIG. 248B shows cartoon of animals implanted with tumors in which 15% of the tumors express high levels of MUC1* and those tumor cells fluoresce green. FIG. 248C shows cartoon of animals implanted with tumors in which 7.5% of the tumors express high levels of MUC1* and those tumor cells fluoresce green. FIG. 248D lists the variables used in these experiments.

FIG. 249A-247F shows cartoons of the experimental strategy and data. FIG. 249A shows cartoons of MUC1 full-length as it appears on normal epithelial cells. FIG. 249B shows four tissue specimens stained with huMNC2-scFv-Fc. FIG. 249C shows cartoons depicting heterogeneous tumors expressing either high (30%-left) or low (7.5%-right) percentages of high MUC1* expressing tumor cells. FIG. 249D shows flow cytometry verifying the percentages of the heterogeneous tumors before their implantation. FIG. 249E shows bar graphs of bioluminescence measured on an IVIS instrument for animals implanted with 30% high MUC1* cells and treated with the various CAR T cells at an effector to target ratio of 10:1 (top) or 1:1 (bottom). FIG. 249F shows bar graphs of bioluminescence measured on an IVIS instrument for animals implanted with 7.5% high MUC1* cells and treated with the various CAR T cells at an effector to target ratio of 10:1 (top) or 1:1 (bottom).

FIG. 250A-250F shows bar graphs of bioluminescence of the tumors measured on an IVIS instrument for animals implanted with 30% high MUC1* cells or 7.5% high MUC1* tumors and treated with the various CAR T cells at various effector to target ratios. FIG. 250A shows IVIS graph of animals implanted with tumors in which 30% expressed high levels of MUC1* and where animals were treated with CAR T cells at an effector to target ratio of 10:1. FIG. 250B shows IVIS graph where effector to target ratio was 5:1. FIG. 250C shows IVIS graph where effector to target ratio was 1:1. FIG. 250D shows IVIS graph of animals implanted with tumors in which 7.5% expressed high levels of MUC1* and where animals were treated with CAR T cells at an effector to target ratio of 10:1. FIG. 250E shows IVIS graph where effector to target ratio was 5:1. FIG. 250F shows IVIS graph where effector to target ratio was 1:1.

FIG. 251A-251D shows photographs of bioluminescence of the tumors measured on an IVIS instrument for animals implanted with 30% high MUC1* cells or 7.5% high MUC1* tumors and treated with the various CAR T cells at effector to target ratios of 10:1 or 1:1. FIG. 251A shows IVIS photographs for animals implanted with tumors in which 30% expressed high levels of MUC1* and where animals were treated with of CAR T cells at an effector to target ratio of 10:1. FIG. 250B shows IVIS photographs where effector to target ratio was 1:1. FIG. 251C shows IVIS photographs for animals implanted with tumors in which 7.5% expressed high levels of MUC1* and where animals were treated with of CAR T cells at an effector to target ratio of 10:1. FIG. 250D shows IVIS photographs where effector to target ratio was 1:1.

FIG. 252A-252D shows magnified fluorescent photographs of dissociated tumors excised from animals implanted with tumors made up of either 30% or 7.5% T47D-MUC1* high antigen density cells and the remainder are low antigen density cells. Animals were treated with various CAR T cells at effector to target ratios of either 10:1 or 1:1. Shown are overlays of bright field images and fluorescent images, wherein the red fluorescence, mCherry, shows the low antigen density cells and the green fluorescence, GFP, shows the low antigen density cells. FIG. 252A shows tumor cells excised from animals implanted with 30% high antigen density tumors and treated with various CAR T cells at effector to target ratio of 10:1. FIG. 252B shows tumor cells excised from animals implanted with 30% high antigen density tumors and treated with various CAR T cells at effector to target ratio of 1:1. FIG. 252C shows tumor cells excised from animals implanted with 7.5% high antigen density tumors and treated with various CAR T cells at effector to target ratio of 10:1. FIG. 252D shows tumor cells excised from animals implanted with 7.5% high antigen density tumors and treated with various CAR T cells at effector to target ratio of 1:1.

FIG. 253A-253H shows cartoons of the experimental strategy and data. FIG. 253A shows cartoons of MUC1 full-length as it appears on normal epithelial cells. FIG. 253B shows four tissue specimens stained with huMNC2-scFv-Fc. FIG. 253C shows cartoons depicting heterogeneous tumors expressing either high or low percentages of high MUC1* expressing tumor cells. FIG. 253D shows flow cytometry verifying 15% percent of the tumor cells expressed high levels of MUC1* before their implantation. FIG. 253E shows bar graphs of bioluminescence measured on an IVIS instrument for animals implanted with 15% high MUC1* cells and treated with the various CAR T cells at an effector to target ratio of 10:1. FIG. 253F shows immunofluorescent photographs of the excised tumors for animals implanted with 15% high antigen density tumors and treated with the various CAR Ts at an effector to target ratio of 10:1. FIG. 253G shows bar graphs of bioluminescence measured on an IVIS instrument for animals implanted with 15% high MUC1* cells and treated with the various CAR T cells at an effector to target ratio of 1:1. FIG. 253H shows immunofluorescent photographs of the excised tumors for animals implanted with 15% high antigen density tumors and treated with the various CAR Ts at an effector to target ratio of 1:1.

FIG. 254A-254B shows bar graphs of bioluminescence measured on an IVIS instrument for animals implanted with 15% high MUC1* cells and treated with the various CAR T cells at an effector to target ratio of 10:1. FIG. 254A shows the graph of animals treated at an effector to target ratio of 10:1. FIG. 254B shows the graph of animals treated at an effector to target ratio of 1:1.

FIG. 255A-255B shows photographs of bioluminescence of tumors measured on an IVIS instrument for animals implanted with 15% high MUC1* cells and treated with the various CAR T cells at an effector to target ratio of 10:1. FIG. 255A shows the photographs of animals treated at an effector to target ratio of 10:1. FIG. 255B shows the photographs of animals treated at an effector to target ratio of 1:1.

FIG. 256A-256B shows fluorescent photographs of the excised tumors showing the mCherry positive, low antigen density cells in red and the GFP positive, high antigen density cells in green. Animals were all implanted with a mixture of tumor cells that before implantation were 15% high MUC1* expressing cells. FIG. 256A shows the photographs of animals treated at an effector to target ratio of 10:1. FIG. 256B shows the photographs of animals treated at an effector to target ratio of 1:1.

FIG. 257 shows a table of results of flow cytometry in which cells of the excised tumors were assayed for the presence of human T cells and CAR T cells, which were then enumerated and analyzed for the presence of markers of T cell exhaustion. Shown here is the analysis of tumors excised from animals that had been implanted with tumors that were 30% high antigen expressing cells, wherein animals were treated with various CAR T cells and an effector to target ratio of 10:1. When the number of CAR T cells detected was less than 25, that number is shown in red and the further analysis of those few cells is called into question.

FIG. 258 shows a table of results of flow cytometry in which cells of the excised tumors were assayed for the presence of human T cells and CAR T cells, which were then enumerated and analyzed for the presence of markers of T cell exhaustion. Shown here is the analysis of tumors excised from animals that had been implanted with tumors that were 15% high antigen expressing cells, wherein animals were treated with various CAR T cells and an effector to target ratio of 10:1. When the number of CAR T cells detected was less than 25, that number is shown in red and the further analysis of those few cells is called into question.

FIG. 259 shows a table of results of flow cytometry in which cells of the excised tumors were assayed for the presence of human T cells and CAR T cells, which were then enumerated and analyzed for the presence of markers of T cell exhaustion. Shown here is the analysis of tumors excised from animals that had been implanted with tumors that were 7.5% high antigen expressing cells, wherein animals were treated with various CAR T cells and an effector to target ratio of 10:1. When the number of CAR T cells detected was less than 25, that number is shown in red and the further analysis of those few cells is called into question.

FIG. 260 shows a table of results of flow cytometry in which cells of the excised tumors were assayed for the presence of human T cells and CAR T cells, which were then enumerated and analyzed for the presence of markers of T cell exhaustion. Shown here is the analysis of tumors excised from animals that had been implanted with tumors that were 30% high antigen expressing cells, wherein animals were treated with various CAR T cells and an effector to target ratio of 5:1. When the number of CAR T cells detected was less than 25, that number is shown in red and the further analysis of those few cells is called into question.

FIG. 261 shows a table of results of flow cytometry in which cells of the excised tumors were assayed for the presence of human T cells and CAR T cells, which were then enumerated and analyzed for the presence of markers of T cell exhaustion. Shown here is the analysis of tumors excised from animals that had been implanted with tumors that were 7.5% high antigen expressing cells, wherein animals were treated with various CAR T cells and an effector to target ratio of 5:1. When the number of CAR T cells detected was less than 25, that number is shown in red and the further analysis of those few cells is called into question.

FIG. 262 shows a table of results of flow cytometry in which cells of the excised tumors were assayed for the presence of human T cells and CAR T cells, which were then enumerated and analyzed for the presence of markers of T cell exhaustion. Shown here is the analysis of tumors excised from animals that had been implanted with tumors that were 30% high antigen expressing cells, wherein animals were treated with various CAR T cells and an effector to target ratio of 1:1. When the number of CAR T cells detected was less than 25, that number is shown in red and the further analysis of those few cells is called into question.

FIG. 263 shows a table of results of flow cytometry in which cells of the excised tumors were assayed for the presence of human T cells and CAR T cells, which were then enumerated and analyzed for the presence of markers of T cell exhaustion. Shown here is the analysis of tumors excised from animals that had been implanted with tumors that were 15% high antigen expressing cells, wherein animals were treated with various CAR T cells and an effector to target ratio of 1:1. When the number of CAR T cells detected was less than 25, that number is shown in red and the further analysis of those few cells is called into question.

FIG. 264 shows a table of results of flow cytometry in which cells of the excised tumors were assayed for the presence of human T cells and CAR T cells, which were then enumerated and analyzed for the presence of markers of T cell exhaustion. Shown here is the analysis of tumors excised from animals that had been implanted with tumors that were 7.5% high antigen expressing cells, wherein animals were treated with various CAR T cells and an effector to target ratio of 1:1. When the number of CAR T cells detected was less than 25, that number is shown in red and the further analysis of those few cells is called into question.

FIG. 265 shows a table of results of flow cytometry in which cells of the excised spleens of the treated animals were assayed for the presence of human T cells and CAR T cells, which were then enumerated and analyzed for the presence of markers of T cell exhaustion. Shown here is the analysis of tumors excised from animals that had been implanted with tumors that were 30% high antigen expressing cells, wherein animals were treated with various CAR T cells and an effector to target ratio of 10:1. When the number of CAR T cells detected was less than 25, that number is shown in red and the further analysis of those few cells is called into question.

FIG. 266 shows a table of results of flow cytometry in which cells of the excised spleens of the treated animals were assayed for the presence of human T cells and CAR T cells, which were then enumerated and analyzed for the presence of markers of T cell exhaustion. Shown here is the analysis of tumors excised from animals that had been implanted with tumors that were 15% high antigen expressing cells, wherein animals were treated with various CAR T cells and an effector to target ratio of 10:1. When the number of CAR T cells detected was less than 25, that number is shown in red and the further analysis of those few cells is called into question.

FIG. 267 shows a table of results of flow cytometry in which cells of the excised spleens of the treated animals were assayed for the presence of human T cells and CAR T cells, which were then enumerated and analyzed for the presence of markers of T cell exhaustion. Shown here is the analysis of tumors excised from animals that had been implanted with tumors that were 7.5% high antigen expressing cells, wherein animals were treated with various CAR T cells and an effector to target ratio of 10:1. When the number of CAR T cells detected was less than 25, that number is shown in red and the further analysis of those few cells is called into question.

FIG. 268 shows a table of results of flow cytometry in which cells of the excised spleens of the treated animals were assayed for the presence of human T cells and CAR T cells, which were then enumerated and analyzed for the presence of markers of T cell exhaustion. Shown here is the analysis of tumors excised from animals that had been implanted with tumors that were 30% high antigen expressing cells, wherein animals were treated with various CAR T cells and an effector to target ratio of 5:1. When the number of CAR T cells detected was less than 25, that number is shown in red and the further analysis of those few cells is called into question.

FIG. 269 shows a table of results of flow cytometry in which cells of the excised spleens of the treated animals were assayed for the presence of human T cells and CAR T cells, which were then enumerated and analyzed for the presence of markers of T cell exhaustion. Shown here is the analysis of tumors excised from animals that had been implanted with tumors that were 15% high antigen expressing cells, wherein animals were treated with various CAR T cells and an effector to target ratio of 5:1. When the number of CAR T cells detected was less than 25, that number is shown in red and the further analysis of those few cells is called into question.

FIG. 270 shows a table of results of flow cytometry in which cells of the excised spleens of the treated animals were assayed for the presence of human T cells and CAR T cells, which were then enumerated and analyzed for the presence of markers of T cell exhaustion. Shown here is the analysis of tumors excised from animals that had been implanted with tumors that were 7.5% high antigen expressing cells, wherein animals were treated with various CAR T cells and an effector to target ratio of 5:1. When the number of CAR T cells detected was less than 25, that number is shown in red and the further analysis of those few cells is called into question.

FIG. 271 shows a table of results of flow cytometry in which cells of the excised spleens of the treated animals were assayed for the presence of human T cells and CAR T cells, which were then enumerated and analyzed for the presence of markers of T cell exhaustion. Shown here is the analysis of tumors excised from animals that had been implanted with tumors that were 30% high antigen expressing cells, wherein animals were treated with various CAR T cells and an effector to target ratio of 1:1. When the number of CAR T cells detected was less than 25, that number is shown in red and the further analysis of those few cells is called into question.

FIG. 272 shows a table of results of flow cytometry in which cells of the excised spleens of the treated animals were assayed for the presence of human T cells and CAR T cells, which were then enumerated and analyzed for the presence of markers of T cell exhaustion. Shown here is the analysis of tumors excised from animals that had been implanted with tumors that were 15% high antigen expressing cells, wherein animals were treated with various CAR T cells and an effector to target ratio of 1:1. When the number of CAR T cells detected was less than 25, that number is shown in red and the further analysis of those few cells is called into question.

FIG. 273 shows a table of results of flow cytometry in which cells of the excised spleens of the treated animals were assayed for the presence of human T cells and CAR T cells, which were then enumerated and analyzed for the presence of markers of T cell exhaustion. Shown here is the analysis of tumors excised from animals that had been implanted with tumors that were 7.5% high antigen expressing cells, wherein animals were treated with various CAR T cells and an effector to target ratio of 1:1. When the number of CAR T cells detected was less than 25, that number is shown in red and the further analysis of those few cells is called into question.

FIG. 274 shows a table of results of flow cytometry in which blood from the treated animals were assayed for the presence of human T cells and CAR T cells, which were then enumerated and analyzed for the presence of markers of T cell exhaustion. Shown here is the analysis of tumors excised from animals that had been implanted with tumors that were 30% high antigen expressing cells, wherein animals were treated with various CAR T cells and an effector to target ratio of 10:1. When the number of CAR T cells detected was less than 25, that number is shown in red and the further analysis of those few cells is called into question.

FIG. 275 shows a table of results of flow cytometry in which blood from the treated animals were assayed for the presence of human T cells and CAR T cells, which were then enumerated and analyzed for the presence of markers of T cell exhaustion. Shown here is the analysis of tumors excised from animals that had been implanted with tumors that were 15% high antigen expressing cells, wherein animals were treated with various CAR T cells and an effector to target ratio of 10:1. When the number of CAR T cells detected was less than 25, that number is shown in red and the further analysis of those few cells is called into question.

FIG. 276 shows a table of results of flow cytometry in which blood from the treated animals were assayed for the presence of human T cells and CAR T cells, which were then enumerated and analyzed for the presence of markers of T cell exhaustion. Shown here is the analysis of tumors excised from animals that had been implanted with tumors that were 7.5% high antigen expressing cells, wherein animals were treated with various CAR T cells and an effector to target ratio of 10:1. When the number of CAR T cells detected was less than 25, that number is shown in red and the further analysis of those few cells is called into question.

FIG. 277 shows a table of results of flow cytometry in which blood from the treated animals were assayed for the presence of human T cells and CAR T cells, which were then enumerated and analyzed for the presence of markers of T cell exhaustion. Shown here is the analysis of tumors excised from animals that had been implanted with tumors that were 30% high antigen expressing cells, wherein animals were treated with various CAR T cells and an effector to target ratio of 5:1. When the number of CAR T cells detected was less than 25, that number is shown in red and the further analysis of those few cells is called into question.

FIG. 278 shows a table of results of flow cytometry in which blood from the treated animals were assayed for the presence of human T cells and CAR T cells, which were then enumerated and analyzed for the presence of markers of T cell exhaustion. Shown here is the analysis of tumors excised from animals that had been implanted with tumors that were 15% high antigen expressing cells, wherein animals were treated with various CAR T cells and an effector to target ratio of 5:1. When the number of CAR T cells detected was less than 25, that number is shown in red and the further analysis of those few cells is called into question.

FIG. 279 shows a table of results of flow cytometry in which blood from the treated animals were assayed for the presence of human T cells and CAR T cells, which were then enumerated and analyzed for the presence of markers of T cell exhaustion. Shown here is the analysis of tumors excised from animals that had been implanted with tumors that were 7.5% high antigen expressing cells, wherein animals were treated with various CAR T cells and an effector to target ratio of 5:1. When the number of CAR T cells detected was less than 25, that number is shown in red and the further analysis of those few cells is called into question.

FIG. 280 shows a table of results of flow cytometry in which blood from the treated animals were assayed for the presence of human T cells and CAR T cells, which were then enumerated and analyzed for the presence of markers of T cell exhaustion. Shown here is the analysis of tumors excised from animals that had been implanted with tumors that were 30% high antigen expressing cells, wherein animals were treated with various CAR T cells and an effector to target ratio of 1:1. When the number of CAR T cells detected was less than 25, that number is shown in red and the further analysis of those few cells is called into question.

FIG. 281 shows a table of results of flow cytometry in which blood from the treated animals were assayed for the presence of human T cells and CAR T cells, which were then enumerated and analyzed for the presence of markers of T cell exhaustion. Shown here is the analysis of tumors excised from animals that had been implanted with tumors that were 15% high antigen expressing cells, wherein animals were treated with various CAR T cells and an effector to target ratio of 1:1. When the number of CAR T cells detected was less than 25, that number is shown in red and the further analysis of those few cells is called into question.

FIG. 282 shows a table of results of flow cytometry in which blood from the treated animals were assayed for the presence of human T cells and CAR T cells, which were then enumerated and analyzed for the presence of markers of T cell exhaustion. Shown here is the analysis of tumors excised from animals that had been implanted with tumors that were 7.5% high antigen expressing cells, wherein animals were treated with various CAR T cells and an effector to target ratio of 1:1. When the number of CAR T cells detected was less than 25, that number is shown in red and the further analysis of those few cells is called into question.

FIG. 283A-283L shows photographs of MUC1* positive breast cancer cells, T47D, in culture with human T cells to which have been added various concentrations of bispecific antibody 20A10-OKT3-BiTE. 20A10 is a humanized anti-MUC1* antibody and OKT3 is an antibody that binds to CD3 that is present on human T cells. As can be seen in the figure, the addition of the bispecific antibody mediates the joining together of T cells and cancer cells, seen here as cell clustering. In FIG. 283A the concentration of the bispecific antibody is 1,000 ng/mL. In FIG. 283B concentration is 333 ng/mL. In FIG. 283C concentration is 111 ng/mL. In FIG. 283D concentration is 37 ng/mL. In FIG. 283E concentration is 12.3 ng/mL. In FIG. 283F concentration is 4.1 ng/mL. In FIG. 283G concentration is 1.3 ng/mL. In FIG. 283H concentration is 0.4 ng/mL. In FIG. 283I concentration is 0.15 ng/mL. In FIG. 283J concentration is 0.05 ng/mL. FIG. 283K is a control well in which both T cells and cancer cells are present, but no bispecific antibody has been added. FIG. 283L is a control well in which bispecific antibody has been added to cancer cells, but no T cells are present.

FIG. 284A-284L shows photographs of MUC1* positive breast cancer cells, T47D, in culture with human T cells to which have been added various concentrations of bispecific antibody 20A10-12F6-BiTE. 20A10 is a humanized anti-MUC1* antibody and 12F6 is an antibody that binds to CD3 that is present on human T cells. As can be seen in the figure, the addition of the bispecific antibody mediates the joining together of T cells and cancer cells, seen here as cell clustering. In FIG. 284A the concentration of the bispecific antibody is 1,000 ng/mL. In FIG. 284B concentration is 333 ng/mL. In FIG. 284C concentration is 111 ng/mL. In FIG. 284D concentration is 37 ng/mL. In FIG. 284E concentration is 12.3 ng/mL. In FIG. 284F concentration is 4.1 ng/mL. In FIG. 284G concentration is 1.3 ng/mL. In FIG. 284H concentration is 0.4 ng/mL. In FIG. 284I concentration is 0.15 ng/mL. In FIG. 284J concentration is 0.05 ng/mL. FIG. 284K is a control well in which both T cells and cancer cells are present, but no bispecific antibody has been added. FIG. 284L is a control well in which bispecific antibody has been added to cancer cells, but no T cells are present.

FIG. 285A-285L shows photographs of HCT-MUC1*-transduced cancer cells in culture with human T cells to which have been added various concentrations of bispecific antibody 20A10-OKT3-BiTE. 20A10 is a humanized anti-MUC1* antibody and OKT3 is an antibody that binds to CD3 that is present on human T cells. As can be seen in the figure, the addition of the bispecific antibody mediates the joining together of T cells and cancer cells, seen here as cell clustering. In FIG. 285A the concentration of the bispecific antibody is 1,000 ng/mL. In FIG. 285B concentration is 333 ng/mL. In FIG. 285C concentration is 111 ng/mL. In FIG. 285D concentration is 37 ng/mL. In FIG. 285E concentration is 12.3 ng/mL. In FIG. 285F concentration is 4.1 ng/mL. In FIG. 285G concentration is 1.3 ng/mL. In FIG. 285H concentration is 0.4 ng/mL. In FIG. 285I concentration is 0.15 ng/mL. In FIG. 285J concentration is 0.05 ng/mL. FIG. 285K is a control well in which both T cells and cancer cells are present, but no bispecific antibody has been added. FIG. 285L is a control well in which bispecific antibody has been added to cancer cells, but no T cells are present.

FIG. 286A-286L shows photographs of HCT-MUC1*-transduced cancer cells in culture with human T cells to which have been added various concentrations of bispecific antibody 20A10-12F6-BiTE. 20A10 is a humanized anti-MUC1* antibody and 12F6 is an antibody that binds to CD3 that is present on human T cells. As can be seen in the figure, the addition of the bispecific antibody mediates the joining together of T cells and cancer cells, seen here as cell clustering. In FIG. 286A the concentration of the bispecific antibody is 1,000 ng/mL. In FIG. 286B concentration is 333 ng/mL. In FIG. 286C concentration is 111 ng/mL. In FIG. 286D concentration is 37 ng/mL. In FIG. 286E concentration is 12.3 ng/mL. In FIG. 286F concentration is 4.1 ng/mL. In FIG. 286G concentration is 1.3 ng/mL. In FIG. 286H concentration is 0.4 ng/mL. In FIG. 286I concentration is 0.15 ng/mL. In FIG. 286J concentration is 0.05 ng/mL. FIG. 286K is a control well in which both T cells and cancer cells are present, but no bispecific antibody has been added. FIG. 286L is a control well in which bispecific antibody has been added to cancer cells, but no T cells are present.

### DETAILED DESCRIPTION OF THE PREFERRED EMBODIMENTS

In the present application, “a” and “an” are used to refer to both single and a plurality of objects.

As used herein, occasionally, in short hand, a polypeptide is indicated as being “transduced or transfected” into a cell. In these occurrences, it is understood that the nucleic acid encoding the polypeptide sequence is transduced or transfected into the cell, as it is an impossibility that a polypeptide could be transduced or transfected into a cell.

As used herein, occasionally when referring to number of cells injected into an animal or otherwise contextually wherein the number of cells is referred to, “M” refers to millions, and “K” refers to thousands.

As used herein, interchangeable designations for various monoclonal antibodies are used, such as, “MNC2”, which is interchangeable with “C2”, “Min-C2” and “MNC2”; “MNE6”, which is interchangeable with “E6”, “Min-E6” and “MNE6”; “MNC3”, which is interchangeable with “C3”, “Min-C3” and “MNC3”; and “MNC8”, which is interchangeable with “C8”, “Min-C8” and “MNC8”. The monoclonal antibodies provided herein follow the same convention.

As used herein, “h” or “hu” placed before an antibody construct is short-hand for humanized.

As used herein, the term “antibody-like” means a molecule that may be engineered such that it contains portions of antibodies but is not an antibody that would naturally occur in nature. Examples include but are not limited to CAR (chimeric antigen receptor) T cell technology and the Ylanthia® technology. The CAR technology uses an antibody epitope fused to a portion of a T cell so that the body's immune system is directed to attack a specific target protein or cell. The Ylanthia® technology consists of an “antibody-like” library that is a collection of synthetic human Fabs that are then screened for binding to peptide epitopes from target proteins. The selected Fab regions can then be engineered into a scaffold or framework so that they resemble antibodies.

As used herein, “PSMGFR” is abbreviation for Primary Sequence of the MUC1 Growth Factor Receptor which is identified by SEQ ID NO:2, and thus is not to be confused with a six amino acid sequence. “PSMGFR peptide” or “PSMGFR region” refers to a peptide or region that incorporates the Primary Sequence of the MUC1 Growth Factor Receptor (SEQ ID NO:2).

As used herein, the “MUC1*” extra cellular domain is defined primarily by the PSMGFR sequence (GTINVHDVETQFNQYKTEAASRYNLTISDVSVSDVPFPFSAQSGA (SEQ ID NO:2)). Because the exact site of MUC1 cleavage depends on the enzyme that clips it, and that the cleavage enzyme varies depending on cell type, tissue type or the time in the evolution of the cell, the exact sequence of the MUC1* extra cellular domain may vary at the N-terminus.

Other clipped amino acid sequences may include SNIKFRPGSVVVQLTLAFREGTINVHDVETQFNQYKTEAASRY (SEQ ID NO:620); or SVVVQLTLAFREGTINVHDVETQFNQYKTEAASRY (SEQ ID NO:621).

As used herein, the term “PSMGFR” is an acronym for Primary Sequence of MUC1 Growth Factor Receptor as set forth as GTINVHDVETQFNQYKTEAASRYNLTISDVSVSDVPFPFSAQSGA (SEQ ID NO:2). In this regard, the “N-number” as in “N-10 PSMGFR” or simply “N-10”, “N-15 PSMGFR” or simply “N-15”, or “N-20 PSMGFR” or simply “N-20” refers to the number of amino acid residues that have been deleted at the N-terminal end of PSMGFR. Likewise “C-number” as in “C-10 PSMGFR” or simply “C-10”, “C-15 PSMGFR” or simply “C-15”, or “C-20 PSMGFR” or simply “C-20” refers to the number of amino acid residues that have been deleted at the C-terminal end of PSMGFR. A mixture of deletions and additions is also possible. For instance, N+20/C-27 refers to a peptide fragment of wild-type MUC1 in which 20 amino acids are added to the PSMGFR at the N-terminus and 27 amino acids are deleted from the C-terminus.

As used herein, the “extracellular domain of MUC1*” refers to the extracellular portion of a MUC1 protein that is devoid of the tandem repeat domain. In most cases, MUC1* is a cleavage product wherein the MUC1* portion consists of a short extracellular domain devoid of tandem repeats, a transmembrane domain and a cytoplasmic tail. The precise location of cleavage of MUC1 is not known perhaps because it appears that it can be cleaved by more than one enzyme. The extracellular domain of MUC1* will include most of the PSMGFR sequence but may have an additional 10-20 N-terminal amino acids.

As used herein “sequence identity” means homology in sequence of a particular polypeptide or nucleic acid to a reference sequence of nucleic acid or amino acid such that the function of the homologous peptide is the same as the reference peptide or nucleic acid. Such homology can be so close with the reference peptide such that at times the two sequences may be 90%, 95% or 98% identical yet possess the same function in binding or other biological activities.

As used herein, “MUC1 positive” cell refers to a cell that expresses a gene for MUC1, MUC1-Y or MUC1-Z or other MUC1 variant.

As used herein, “MUC1 negative” cell refers to a cell that does not express a gene for MUC1.

As used herein, “MUC1* positive” cell refers to a cell that expresses a gene for MUC1, wherein that gene's expressed protein is a transmembrane protein that is devoid of tandem repeats, which may be a consequence of post-translational modification, cleavage, alternative splicing, or transfecting or transducing a cell with a MUC1 protein that is devoid of tandem repeats.

As used herein, “MUC1* negative” cell refers to a cell that may or may not express a gene for MUC1 but does not express a MUC1 transmembrane protein that is devoid of tandem repeats.

As used herein, “MUC1 positive” cancer cell refers to a cancer cell that overexpresses the gene for MUC1, expresses MUC1 in an aberrant pattern, wherein its expression is not restricted to the apical border and/or expresses a MUC1 that is devoid of tandem repeats.

As used herein, “MUC1 negative” cancer cell refers to a cancer cell that may or may not express a gene for MUC1 but does not overexpress MUC1 or does not overexpress a MUC1 transmembrane protein that is devoid of tandem repeats.

As used herein, “MUC1* positive” cancer cell refers to a cancer cell that overexpresses a MUC1 transmembrane protein that is devoid of tandem repeats.

As used herein, “MUC1* negative” cancer cell refers to a cancer cell that may or may not express a gene for MUC1 but does not overexpress a MUC1 transmembrane protein that is devoid of tandem repeats.

As used herein “conformational epitope” refers to a peptide sequence that is required to be present in a specific three-dimensional structure or conformation for an antibody to bind. However the antibody binds when the peptide sequence is in the three-dimensional structure or conformation and is not bound when linear. A common technique for determining whether an antibody binds to a linear stretch or a conformational epitope is to use the antibody to probe a denaturing Western blot. Traveling through a denaturing gel linearizes proteins and peptides. Antibodies that do not work in a denaturing Western but do recognize the native target, for example expressed on an intact cell, are determined to recognize a conformational epitope. As used herein, the antibody may or may not actually bind to the “conformational epitope”, however the presence of the “conformational epitope” sequence is required to render a three dimensional structure so that the MUC1* region on cancer cells is able to be bound by the antibody that is specific for cancer treatment. Thus, the conformational epitope is an amino acid sequence that induces the binding of the antibody to the MUC1* region on cancer cells. Thus, a term “conformational inducing peptide sequence” may be used, which indicates that a peptide sequence is present within a larger peptide not as a binding site but that induces binding of an antibody to the larger peptide by causing a three-dimensional structure to form that facilitates the binding of the antibody to the larger peptide.

### MUC1* Antibodies (Anti-PSMGFR) for Treatment or Prevention of Cancers

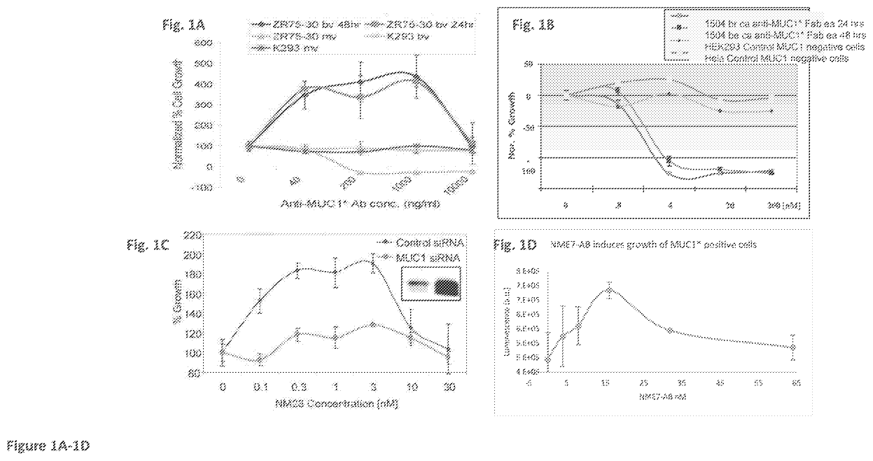

We discovered that a cleaved form of the MUC1 (SEQ ID NO:1) transmembrane protein is a growth factor receptor that drives the growth of over 75% of all human solid tumor cancers. The cleaved form of MUC1, which we called MUC1* (pronounced muk 1 star), is a powerful growth factor receptor. Enzymatic cleavage releases the bulk of the MUC1 extracellular domain. It is the remaining portion comprising a truncated extracellular domain, transmembrane domain and cytoplasmic tail that is called MUC1*. Cleavage and release of the bulk of the extracellular domain of MUC1 unmasks a binding site for activating ligands dimeric NME1, NME6, NME8, NME7_{AB}, NME7-X1 or NME7. Cell growth assays show that it is ligand-induced dimerization of the MUC1* extracellular domain that promotes growth (FIG. 1A-1D). MUC1* positive cells treated with either bivalent ‘by’ anti-MUC1* antibody, monovalent ‘my’ or Fab, NM23-H1 dimers or NME7-AB. Bivalent anti-MUC1* antibodies stimulate growth of cancer cells whereas the monovalent Fab inhibits growth. Classic bell-shaped curve indicates ligand induced dimerization stimulates growth. Dimeric NM23-H1, aka NME1, stimulates growth of MUC1* positive cancer cells but siRNA to suppress MUC1 expression eliminate its effect (FIG. 1C). NME7-AB also stimulates the growth of MUC1* positive cells (FIG. 1D).

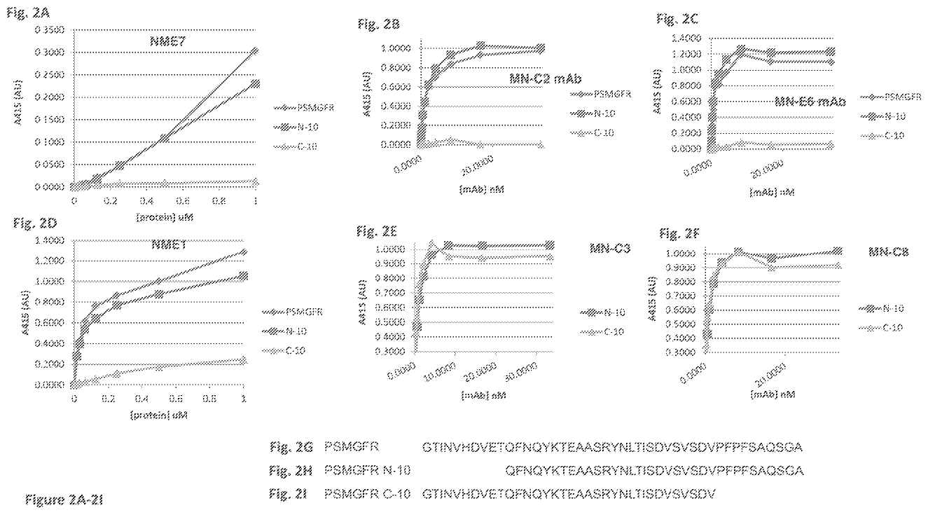

MUC1* is an excellent target for cancer drugs as it is aberrantly expressed on over 75% of all cancers and is likely overexpressed on an even higher percentage of metastatic cancers. After MUC1 cleavage, most of its extracellular domain is shed from the cell surface. The remaining portion has a truncated extracellular domain that at least comprises the primary growth factor receptor sequence, PSMGFR (SEQ ID NO:2). Antibodies that bind to the PSMGFR sequence and especially those that competitively inhibit the binding of activating ligands such as NME proteins, including NME1, NME6, NME8, NME7_{AB}, NME7-X1 and NME7, are ideal therapeutics and can be used to treat or prevent MUC1 positive or MUC1* positive cancers, as stand-alone antibodies, antibody fragments or variable region fragments thereof incorporated into multi-specific antibody-like molecules, bispecific antibodies, antibody-drug conjugates or chimeric antigen receptors also called CARs, which are then transfected or transduced into immune cells, then administered to a patient. Therapeutic anti-MUC1* antibodies can be monoclonal, polyclonal, antibody mimics, engineered antibody-like molecules, full antibodies or antibody fragments. Examples of antibody fragments include but are not limited to Fabs, scFv, and scFv-Fc. Human or humanized antibodies are preferred for use in the treatment or prevention of cancers. In any of these antibody-like molecules, mutations can be introduced to prevent or minimize dimer formation. Anti-MUC1* antibodies that are monovalent or bispecific are preferred because MUC1* function is activated by ligand induced dimerization. Typical binding assays show that NME1 and NME7A bind to the PSMGFR peptide portion of MUC1* (FIG. 2A, 2D). Further, they show that these activating growth factors bind to the membrane proximal portion of MUC1*, as they do not bind to the PSMGFR peptide if the 10 C-terminal amino acids are missing. Similarly, anti-MUC1* antibodies MNC2 and MNE6 bind to the PSMGFR peptide if an only if the 10 C-terminal amino acids are present (FIG. 2B, 2C). Antibodies MNC3 and MNC8 bind to epitopes that are different from MNC2 and MNE6, as they do not depend on the presence of the 10 C-terminal amino acids of the PSMGFR peptide (FIG. 2E, 2F). Antibodies MNC2, MNE6, 20A10, 3C2B1, 5C6F3, 25E6, 18G12, 28F9, 1E4, B12, B2, B7, B9, 8C7F3, and H11 antibody and other antibodies of the invention or fragments derived from them, can be administered to a patient for the treatment or prevention of cancers, as stand-alone antibodies or incorporated into a BiTE, an ADC, a multi-specific antibody-like molecule, bispecific antibodies, with or without an FC region or a portion of an Fc region, a bi-scFv, a di-scFv, a tandem di-scFv, a diabody, triabody, tribody, tetrabody and other antibody-like molecules that are multi-valent and multi-specific. The antibody or antibody fragment may be murine, human, humanized, camelid, rabbit or other non-human species.

BiTEs or chimeric antigen receptors also called CARs that have been transduced into immune cells. MNC2, MNE6, 20A10, 3C2B1, 5C6F3, 25E6, 18G12, 28F9, 1E4, B12, B2, B7, B9, 8C7F3, and H11 antibody and other anti-MUC1* antibodies that competitively inhibit the binding of NME1 and NME7A are preferred. The antibody or antibody fragment may be murine, human, humanized, camelid, rabbit or other non-human species.

Therapeutic anti-MUC1* antibodies for use as a stand-alone antibody therapeutic or for integration into a BiTE, a CAR, an ADC, or any of the multi-specific antibody-like molecules can be selected based on specific criteria. The parent antibody can be generated using typical methods for generating monoclonal antibodies in animals. Alternatively, they can be selected by screening antibody or antibody fragment libraries, including but not limited to strategies described in Beckman U.S. Pat. No. 9,944,719B2, which is incorporated by reference herein for description of methods of screening antibodies. Antibodies suitable for therapeutic use are chosen based on their ability to bind to a MUC1* peptide, which can be:
(i) PSMGFR region of MUC1;
(ii) PSMGFR peptide;
(iii) a peptide having amino acid sequence of QFNQYKTEAASRYNLTISDVSVSDVPFPFSAQSGA (N-10)
(iv) a peptide having amino acid sequence of
ASRYNLTISDVSVSDVPFPFSAQSGA (N-19)
(v) a peptide having amino acid sequence of
NLTISDVSVSDVPFPFSAQSGA (N-23)
(vi) a peptide having amino acid sequence of
ISDVSVSDVPFPFSAQSGA (N-26)
(vii) a peptide having amino acid sequence of
SVSDVPFPFSAQSGA (N-30)
(viii) a peptide having amino acid sequence of
QFNQYKTEAASRYNLTISDVSVSDVPFPFS (N-10/C-5)
(ix) a peptide having amino acid sequence of
ASRYNLTISDVSVSDVPFPFS (N-19/C-5) or
(x) a peptide having amino acid sequence of
FPFSAQSGA (N-36).

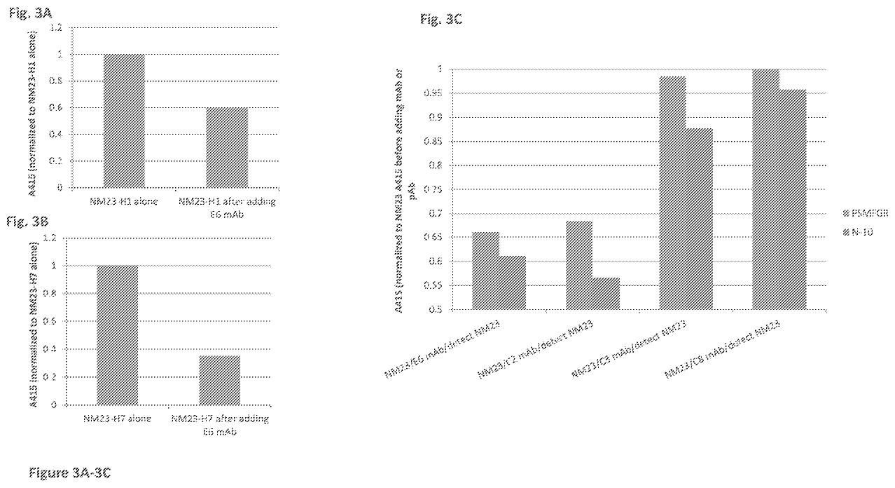

Resultant antibodies or antibody fragments generated or selected in this way can then be further selected by passing additional screens. For example, antibodies or antibody fragments become more preferred based on their ability to bind to MUC1* positive cancer cells or tissues but not to MUC1 negative cancer cells or to normal tissues. Further, anti-MUC1* antibodies or antibody fragments may be de-selected as anti-cancer therapeutics if they bind to stem or progenitor cells. Anti-MUC1* antibodies or antibody fragments become more preferred if they have the ability to competitively inhibit the binding of activating ligands, such as NME7_{AB} or NME7-X1, to MUC1*. FIGS. 3A-3C shows that MNE6 and MNC2 competitively inhibit the binding of activating ligands NME1 and NME7 to MUC1*.

A process for selecting anti-MUC1* antibodies for use in treating a patient diagnosed with a MUC1 positive cancer, at risk of developing a MUC1 positive cancer or suspected of having a MUC1 positive cancer comprises one or more of the following steps of selecting antibodies or antibody fragments that 1) bind to the PSMGFR peptide; 2) bind to the N-10 PSMGFR peptide; 3) selectively bind to cancer cells; 4) do not bind to C-10 PSMGFR peptide; and 5) competitively inhibited the binding of dimeric NME1 or NME7-AB to the PSMGFR peptide. For example, FIGS. 3A-3C show that monoclonals MNE6 and MNC2 satisfy all five criteria, while monoclonals MNC3 and MNC8 do not competitively inhibit the binding of activating ligands NME1 and NME7 (FIG. 3C). Recall that the MUC1* growth factor receptor is activated by ligand-induced dimerization of its extracellular domain. Therefore, the ideal antibody therapeutic, if used as a straight stand-alone antibody therapeutic, should not dimerize the MUC1* extracellular domain. For this therapeutic format, suitable antibodies in this regard include monovalent antibodies such as those generated in lamas and camels, Fabs, scFv's, single domain antibodies (sdAb), scFv-Fc as long as the Fc portion is constructed such that it does not homo-dimerize.

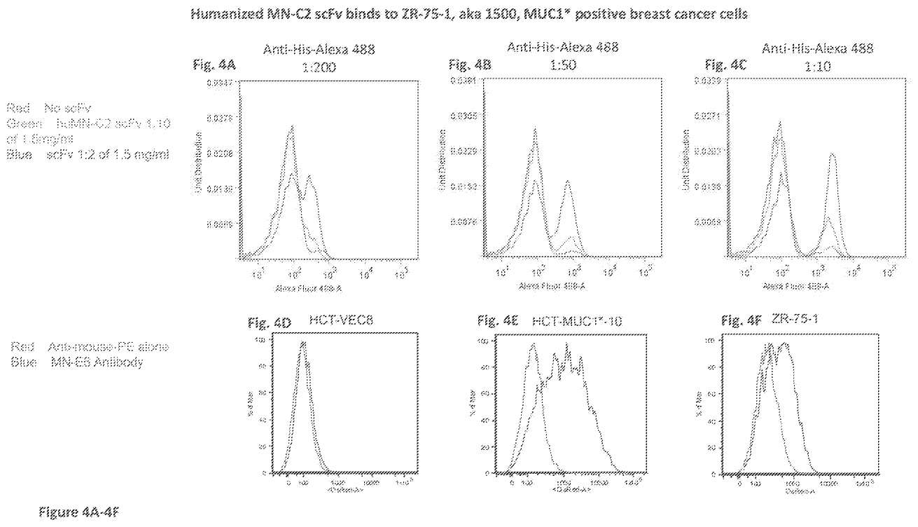

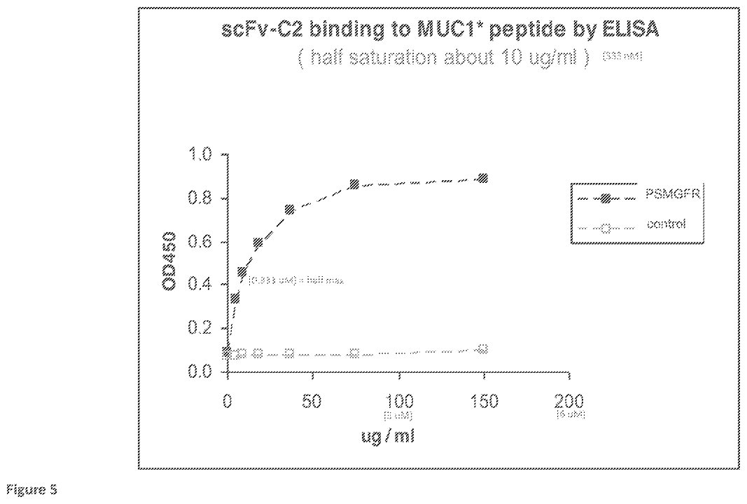

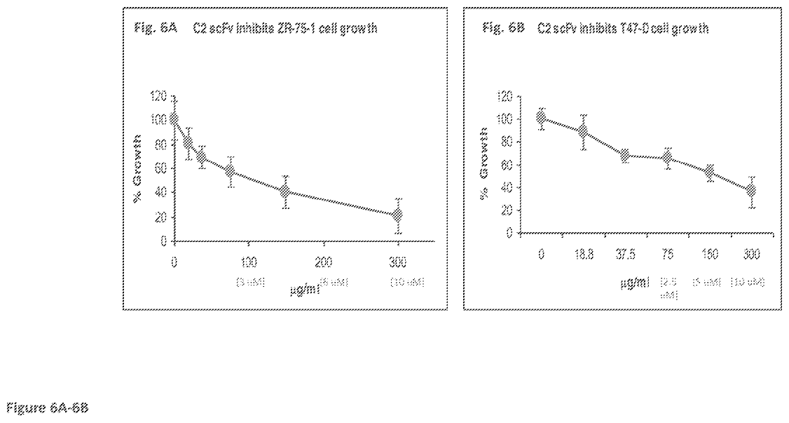

FACS scans show that anti-MUC1* antibodies MNC2 and MNE6 specifically bind to MUC1* positive solid tumor cancer cells and MUC1* transfected cells but not MUC1* negative or MUC1 negative cells. In one example, a humanized MNC2 scFv is shown to bind to ZR-75-1, aka 1500, MUC1* positive breast cancer cells (FIG. 4A-4C). MNE6 was shown to bind to MUC1 negative HCT-116 colon cancer cells if an only if they were transfected with MUC1*. MNE6 also bound to MUC1* positive cancer cells such as ZR-75-1, aka 1500, MUC1* positive breast cancer cells (FIG. 4D-4F). Binding assays such as ELISAs, immunofluorescence, and the like all confirm that MNC2 and MNE6 bind to the PSMGFR peptide and to live MUC1 positive cancer cells. Humanized anti-MUC1* antibodies are selected based on their ability to also bind to the PSMGFR peptide or to MUC1 positive cancer cells. FIG. 5 shows that humanized MNC2 scFv binds with high affinity to the MUC1* peptide PSMGFR with an EC-50 of about 333 nM. Humanized MNC2 scFv, like Fabs, potently inhibits the growth of MUC1* positive cancer cells as is shown in one example in FIGS. 6A, 6B. Like the parent antibodies, humanized scFv's show the same binding pattern. huMNE6-scFv binds to the PSMGFR peptide, binds to the N-10 peptide but does not bind to the C-10 peptide (SEQ ID NO:825) (FIG. 8). However, murine or humanized MNC3-scFv, which is less suitable for the treatment of cancers, binds to the, PSMGFR peptide, binds to the N-10 peptide and also binds to the C-10 peptide (FIG. 9), which we know is the epitope to which the activating ligand NME7_{AB} binds.

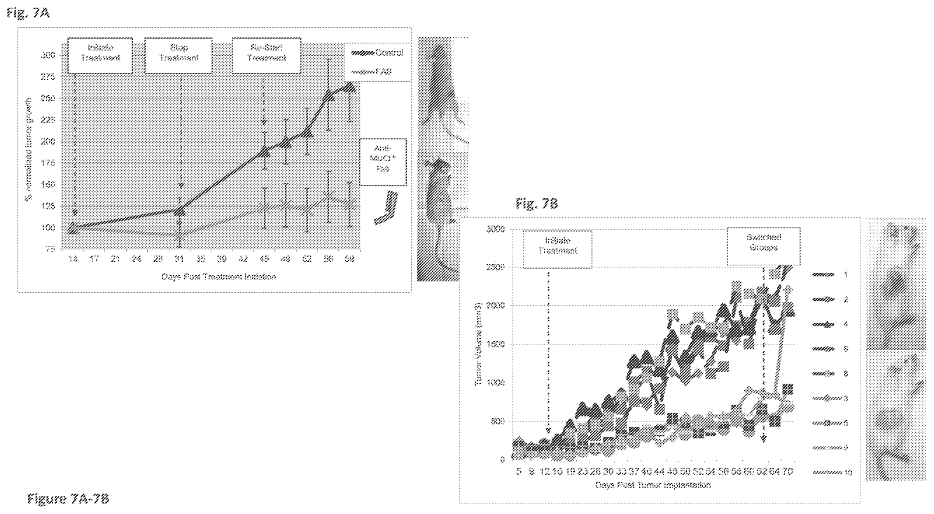

The Fabs of MNE6 and MNC2 or the comparable single chain variable regions derived from them potently inhibit the growth of MUC1* positive cancers in vitro and in vivo. In several examples, the Fabs of Anti-MUC1* antibodies inhibited the growth of human MUC1* positive cancers in vivo. In one case, immune-compromised mice were implanted with human breast tumors then treated with MNE6 Fab after tumor engraftment. FIG. 7A shows that MNE6 Fab potently inhibited the growth of MUC1* positive breast cancers. Female nu/nu mice implanted with 90-day estrogen pellets were implanted with 6 million T47D human breast cancer cells that had been mixed 50/50 with Matrigel. Mice bearing tumors that were at least 150 mm³ and had three successive increases in tumor volume were selected for treatment. Animals were injected sub-cutaneously twice per week with 80 mg/kg MNE6 Fab and an equal number of mice fitting the same selection criteria were injected with vehicle alone (FIG. 7A).

In another aspect, MNE6 was shown to halt the growth of prostate cancer. FIG. 7B shows that MNE6 Fab potently inhibited the growth of MUC1* positive prostate cancers. Male NOD/SCID mice were implanted with 6 million DU-145 human prostate cancer cells that had been mixed 50/50 with Matrigel. Mice bearing tumors that were at least 150 mm\hat{ }3 and had three successive increases in tumor volume were selected for treatment. Animals were injected sub-cutaneously every 48 hours with 160 mg/kg MNE6 Fab and an equal number of mice fitting the same selection criteria were injected with vehicle alone (FIG. 7B). Tumors were measured independently by two researchers twice per week and recorded. Statistics were blindly calculated by independent statistician, giving a P value of 0.0001 for each. Anti-MUC1* Fab inhibited breast cancer growth and prostate cancer growth. Treatment had no effect on weight, bone marrow cell type or number. The MNE6 Fab effectively inhibited the growth of the tumors, while the control group's tumors continued to grow until sacrifice. No adverse effects of treatment were observed or detected.

Recombinant forms of MNE6 and MNC2 were constructed that like the Fab are monomeric. In this case, MNE6 was humanized and MNC2 was humanized. There are a number of methods known to those skilled in the art for humanizing antibodies. In addition to humanizing, libraries of human antibodies or antibody fragments can be screened to identify other fully human antibodies that bind to the PSMGFR.

A single chain of the humanized MNE6 variable region, called an scFv, was genetically engineered such that it was connected to the Fc portion of the antibody (SEQ ID NO:256 and 257). Fc regions impart certain benefits to antibody fragments for use as therapeutics. The Fc portion of an antibody recruits complement, which in general means it can recruit other aspects of the immune system and thus amplify the anti-tumor response beyond just inhibiting the target. The addition of the Fc portion also increases the half-life of the antibody fragment (Czajkowsky D M, Hu J, Shao Z and Pleass R J. (2012) Fc-fusion proteins: new developments and future perspectives. EMBO Mol Med. 4(10):1015-1028). However, the Fc portion of an antibody homo-dimerizes, which in the case of anti-MUC1* antibody based therapeutics is not optimal since ligand-induced dimerization of the MUC1* receptor stimulates growth. Therefore, mutations in the Fc region that resist dimer formation are preferred for anti-MUC1* anti-cancer therapeutics. Deletion of the hinge region and other mutations in the Fc region that make the Fc-mutant resistant to dimerization were made and could be used as therapeutics.

A human or humanized MNE6 antibody or antibody fragment, Fab, MNE6 scFv or hu MNE6 scFv-Fcₘᵤₜ are effective anti-cancer agents that can be administered to a person diagnosed with a MUC1 or MUC1* positive cancer, suspected of having a MUC1 or MUC1* positive cancer or is at risk of developing a MUC1 or MUC1* positive cancer.

### Humanizing

Humanized antibodies or antibody fragments or fully human antibodies that bind to the extracellular domain of -MUC1* are preferred for therapeutic use. The techniques described herein for humanizing antibodies are but a few of a variety of methods known to those skilled in the art. The invention is not meant to be limited by the technique used to humanize the antibody.

Humanization is the process of replacing the non-human regions of a therapeutic antibody (usually mouse monoclonal antibody) by human one without changing its binding specificity and affinity. The main goal of humanization is to reduce immunogenicity of the therapeutic monoclonal antibody when administered to human. Three distinct types of humanization are possible. First, a chimeric antibody is made by replacing the non-human constant region of the antibody by the human constant region. Such antibody will contain the mouse Fab region and will contain about 80-90% of human sequence. Second, a humanized antibody is made by grafting of the mouse CDR regions (responsible of the binding specificity) onto the variable region of a human antibody, replacing the human CDR (CDR-grafting method). Such antibody will contain about 90-95% of human sequence. Third and last, a full human antibody (100% human sequence) can be created by phage display, where a library of human antibodies, antibody-like molecules or antibody fragments is screened to select antigen specific human antibody or by immunizing transgenic mice expressing human antibody.

A general technique for humanizing an antibody is practiced approximately as follows. Monoclonal antibodies are generated in a host animal, typically in mice. Monoclonal antibodies are then screened for affinity and specificity of binding to the target. Once a monoclonal antibody that has the desired effect and desired characteristics is identified, it is sequenced. The sequence of the animal-generated antibody is then aligned with the sequences of many human antibodies in order to find human antibodies with sequences that are the most homologous to the animal antibody. Biochemistry techniques are employed to paste together the human antibody sequences and the animal antibody sequences. Typically, the non-human CDRs are grafted into the human antibodies that have the highest homology to the non-human antibody. This process can generate many candidate humanized antibodies that need to be tested to identify which antibody or antibodies has the desired affinity and specificity.

Once a human antibody or a humanized antibody has been generated it can be further modified for use as an Fab fragment, as a full antibody, or as an antibody-like entity such as a single chain molecule containing the variable regions, such as scFv or an scFv-Fc. In some cases it is desirable to have Fc region of the antibody or antibody-like molecule mutated such that it does not dimerize.

In addition to methods that introduce human sequences into antibodies generated in non-human species, fully human antibodies can be obtained by a variety of methods known to those skilled in the art, including screening human antibody libraries with a peptide fragment of an antigen. A fully human antibody that functions like MNE6 or MNC2, 20A10 or other antibodies of the invention can be generated by screening a human antibody library or library of antibody fragments with a peptide having the sequence of the PSMGFR N-10 peptide. In another method, human antibodies are generated in genetically modified mice. Humanized anti-MUC1* antibodies or antibody fragments were generated based on the sequences of the mouse monoclonal antibodies MNE6, MNC2, 20A10, 3C2B1, 5C6F3 and 25E6. In one aspect of the invention, a patient diagnosed with a MUC1* positive cancer is treated with an effective amount of a murine or camelid antibody or antibody fragment comprising sequences from MNC2 (SEQ ID NO:118-119 and 168-169), MNE6 (SEQ ID NOS: 12-13 and 65-66), 20A10 (SEQ ID NOS:988-989 and 1004-1005), 3C2B1 (SEQ ID NOS:1820-1821 and 1822-1823), 5C6F3 (SEQ ID NO:1816-1817 and 1818-1819), 25E6 (SEQ ID NO:1020-1021 and 1036-1037), 18G12, 28F9, 1E4, B12, B2, B7, B9, 8C7F3, or H11. In another aspect of the invention, a patient diagnosed with a MUC1* positive cancer is treated with an effective amount of human or humanized antibody or antibody fragment comprising sequences from MNE6 (SEQ ID NOS:56-57 and 107-108, or 341-342, or 391-392, or 393-394) or MNC2 (SEQ ID NO:144-145 and 194-195, or 654-655, or 239-249, or 5017-5020), 20A10 (SEQ ID NOS:1576-1581 or 5001-5012), 3C2B1 (SEQ ID NOS:1820-1823 or 1812-1813), 5C6F3 (SEQ ID NOS:1816-1819, or 1814-1815), 25E6 (SEQ ID NOS:1020-1021 and 1036-1037, or 1600-1601), 18G12, 28F9, 1E4, B12, B2, B7, B9, 8C7F3, or H11. In a preferred embodiment, a patient diagnosed with a MUC1* positive cancer is treated with an effective amount of humanized antibody or antibody fragment comprising sequences of MNC2 (SEQ ID NO:654-655), MNE6 (SEQ ID NO:341-342), 20A10 (SEQ ID NO:1580-1581), 3C2B1 (SEQ ID NO:1812-1813), 5C6F3 (SEQ ID NO:1814-1815), 25E6 (SEQ ID NO:1600-1601). In another aspect of the invention, a patient diagnosed with a MUC1* positive cancer is treated with an effective amount of humanized monovalent form of the antibodies such as MNC2 (SEQ ID NOS:239, 241, 243,396 or 5018-5020), MNE6 (SEQ ID NO:), 20A10 (SEQ ID NOS:1574-1581 or SEQ ID NOS:5001-5012), 3C2B1 (SEQ ID NO:1813), 5C6F3 (SEQ ID NO:1815), 25E6, 18G12, 28F9, 1E4, B12, B2, B7, B9, 8C7F3, or H11, wherein monovalent means the corresponding Fab fragment, the corresponding scFv or the corresponding scFv-Fc fusion. In a preferred embodiment, a patient diagnosed with a MUC1* positive cancer is treated with an effective amount of a humanized scFv or monomeric humanized scFv-Fc of MNC2, MNE6, 20A10, 3C2B1, 5C6F3, 25E6, 18G12, 28F9, 1E4, B12, B2, B7, B9, 8C7F3, or H11. Since the MUC1* growth factor receptor is activated by ligand induced dimerization of its extracellular domain, and because the Fc portion of an antibody homo-dimerizes, it is preferable that a construct that includes an Fc portion uses a mutated Fc region that prevents or minimizes dimerization.

Antibodies that bind to PSMGFR (SEQ ID NO:2) peptide, and more specifically to the N-10 peptide, of the extracellular domain of the MUC1* receptor are potent anti-cancer therapeutics that are effective for the treatment or prevention of MUC1* positive cancers. They have been shown to inhibit the binding of activating ligands dimeric NME1 (SEQ ID NO:1781) and NME7_{AB} (SEQ ID NOS:827) to the extracellular domain of MUC1*. Anti-MUC1* antibodies that bind to the PSMGFR sequence inhibit the growth of MUC1*-positive cancer cells, specifically if they inhibit ligand-induced receptor dimerization. Fabs of anti-MUC1* antibodies have been demonstrated to block tumor growth in animals. Thus, antibodies or antibody fragments that bind to the extracellular domain of MUC1* would be beneficial for the treatment of cancers wherein the cancerous tissues express MUC1*.

Antibodies that bind to PSMGFR region of MUC1* or bind to a synthetic PSMGFR peptide are preferred. Especially preferred are antibodies that bind to the N-10 peptide but not to the C-10 peptide. Still more preferred are antibodies that bind to the N-26 peptide wherein mutation or deletion of the PFPFS sequence (SEQ ID NO:1747) destroys binding of the antibody or fragment thereof to the N-26 peptide. We have identified several monoclonal antibodies that bind to the extracellular domain of MUC1*. Among this group are mouse monoclonal antibodies MNC2 (SEQ ID NOS:118-131, 144-158, 163-164, 168-181, 194-209), MNE6 (SEQ ID NOS:12-25, 39-59, 65-78, 93-114), 20A10 (SEQ ID NOS:988-1019, 1574-1597, 1659-1666); 3C2B1 (SEQ ID NOS:1386-1413, 1572-1573), 5C6F3 (SEQ ID NOS:1356-1385), 25E6 (SEQ ID NOS:1020-1051, 1598-1617, 1667-1674), 18G12 (SEQ ID NOS:956-987), 28F9 (SEQ ID NOS:1052-1083), 1E4 (SEQ ID NOS:1116-1227), B12 (SEQ ID NOS:1414-1431, 1733-1742), B2 (SEQ ID NOS:1432-1459), B7 (SEQ ID NOS:1460-1487), B9 SEQ ID NOS:1544-1571), 8C7F3 (SEQ ID NOS:1488-1515), or H11 (SEQ ID NOS:1516-1543), the variable regions of which were sequenced and are given as for MNE6 SEQ ID NOS: 12-13 and 65-66, for MNC2 SEQ ID NOS: 118-119 and 168-169. The CDRs of these antibodies make up the recognition units of the antibodies and are the most important parts of the mouse antibody that should be retained when grafting into a human antibody. The sequences of the CDRs for each mouse monoclonal are as follows, heavy chain sequence followed by light chain: MNE6 CDR1 (SEQ ID NO:16-17 and 69-70) CDR2 (SEQ ID NO:20-21 and 73-74) CDR3 (SEQ ID NO: 24-25 and 77-78), MNC2 CDR1 (SEQ ID NO:122-123 and 172-173) CDR2 (SEQ ID NO:126-127 and 176-177) CDR3 (SEQ ID NO:130-131 and 180-181), 20A10 CDR1 (SEQ ID NO:991-992 and 1008-1009) CDR2 (SEQ ID NO:996-997 and 1012-1013) CDR3 (SEQ ID NO:1000-1001 and 1016-1017), 3C2B1 CDR1 (SEQ ID NO:1388-1389 and 1402-1403) CDR2 (SEQ ID NO:1392-1393 and 1406-1407) CDR3 (SEQ ID NO:1396-1397 and 1410-1411), 5C6F3 CDR1 (SEQ ID NO:1358-1359 and 1372-1373) CDR2 (SEQ ID NO:1362-1363 and 1376-1377) CDR3 (SEQ ID NO:1366-1367 and 1380-1381), and 25E6 CDR1 (SEQ ID NO:1024-1025 and 1040-1041) CDR2 (SEQ ID NO:1028-1029 and 1044-1045) CDR3 (SEQ ID NO:1032-1033 and 1048-1049). In some cases, portions of the framework regions that by modeling are thought to be important for the 3-dimensional structure of the CDRs, are also imported from the mouse sequence.

Monoclonal antibodies MNE6, MNC2, 20A10, 3C2B1 and 25E6 have greater affinity for MUC1* as it appears on cancer cells. Monoclonal antibodies MNC3 and MNC8 have greater affinity for MUC1* as it appears on stem cells.

All seven antibodies have been humanized, which process has resulted in several humanized forms of each antibody. CDRs derived from the variable regions of the mouse antibodies were biochemically grafted into a homologous human antibody variable region sequence. Humanized variable regions of MNE6 (SEQ ID NOS: 38-39 and 93-94), MNC2 (SEQ ID NOS: 144-145 and 194-195), 20A10 (SEQ ID NOS:1576-1581 and 5001-5012), 3C2B1 (SEQ ID NOS:1812-1813), 5C6F3 (SEQ ID NOS: 1814-1815), 25E6 (SEQ ID NOS:1600-1601). MNC3 (SEQ ID NOS: 439-440 and 486-487) and MNC8 (SEQ ID NOS: 525-526 and 543-544) were generated by grafting the mouse CDRs into the variable region of a homologous human antibody. The humanized heavy chain variable constructs were then fused into constant regions of either human IgG1 heavy chain constant region (SEQ ID NOS:58-59) or human IgG2 heavy chain constant region (SEQ ID NO:54-55), which are then paired with either humanized light chain variable constructs fused to a human kappa chain (SEQ ID NO: 109-110) or human lambda chain (SEQ ID NO: 113-114) constant region. Other IgG isotypes could be used as constant region including IgG3 or IgG4.

Examples of humanized MNE6 variable region into an IgG2 heavy chain (SEQ ID NOS:52-53) and into an IgG1 heavy chain (SEQ ID NOS:56-57), humanized MNC2 variable into an IgG1 heavy chain (SEQ ID NOS: 157-158) or into an IgG2 heavy chain (SEQ ID NOS: 163-164) paired with either Lambda light chain (SEQ ID NO: 111-112 and 216-219) or Kappa chain (SEQ ID NO:107-108 and 210-213) and, humanized MNC3 (SEQ ID NOS: 455-456, 453-454 and 500-501, 502-503) and MNC8 (SEQ ID NOS: 541-542, 539-540 and 579-580, 581-582) antibodies were generated. Which IgG constant region is fused to the humanized variable region depends on the desired effect since each isotype has its own characteristic activity. The isotype of the human constant region is selected on the basis of things such as whether antibody dependent cell cytotoxicity (ADCC) or complement dependent cytotoxicity (CDC) is desired but can also depend on the yield of antibody that is generated in cell-based protein expression systems. In a preferred embodiment, humanized anti-MUC1* antibodies or antibody fragments are administered to a person diagnosed with or at risk of developing a MUC1-positive cancer.

One method for testing and selecting the humanized anti-MUC1* antibodies that would be most useful for the treatment of persons with cancer or at risk of developing cancers is to test them for their ability to inhibit the binding of activating ligands to the MUC1* extracellular domain. Dimeric NME1 can bind to and dimerize the MUC1* extracellular domain and in so doing stimulates cancer cell growth. Antibodies and antibody fragments that compete with NME1 for binding to the MUC1* extracellular domain are therefore anti-cancer agents. NME7A is another activating ligand of MUC1*. In some cases, it is preferable to identify antibodies that block the binding of NME7, or an NME7A truncation or cleavage product of NME7-X1, to the MUC1* extracellular domain. Antibodies and antibody fragments that compete with NME7 and NME7 variants for binding to the MUC1* extracellular domain are effective as anti-cancer therapeutics. These antibodies include but are not limited to MNC2, MNE6, 20A10, 3C2B1, 5C6F3, 25E6, 18G12, 28F9, 1E4, B12, B2, B7, B9, 8C7F3, or H11 as well as single chain versions, such as scFv, of these antibodies and humanized version thereof. Other NME proteins also bind to MUC1 or MUC1* including NME1, NME6 and NME8. Antibodies that compete with these proteins for binding to MUC1* may also be useful as therapeutics. In a preferred embodiment, murine, camelid, human or humanized anti-MUC1* antibodies or antibody fragments are administered to a person diagnosed with or at risk of developing a MUC1-positive cancer. In a more preferred embodiment, single chain antibody fragments, or monomeric scFv-Fc fusions, derived from humanized sequences of MNC2, MNE6, 20A10, 3C2B1, 5C6F3, 25E6, 18G12, 28F9, 1E4, B12, B2, B7, B9, 8C7F3, or H11 are administered to a person diagnosed with or at risk of developing a MUC1-positive cancer.

Single chain variable fragments, scFv, or other forms that result in a monovalent antibody or antibody-like protein are also useful. In some cases it is desired to prevent dimerization of the MUC1* extracellular domain. Single chain variable fragments, Fabs and other monovalent antibody-like proteins have been shown to be effective in binding to the extracellular domain of MUC1* and blocking MUC1* dimerization. These single chain variable fragments, Fabs and other monovalent antibody-like molecules effectively blocked cancer growth in vitro and in animals xenografted with human MUC1-positive cancer cells. Thus, humanized single chain variable fragments or monovalent anti-MUC1* antibodies or antibody-like molecules would be very effective as an anti-cancer therapeutic. Such humanized single chain antibodies, Fabs and other monovalent antibody-like molecules that bind to the MUC1* extracellular domain or to a PSMGFR peptide are therefore useful as anti-cancer therapeutics. Anti-MUC1* single chain variable fragments are generated by grafting non-human CDRs of antibodies, which bind to extracellular domain of MUC1* or bind to PSMGFR peptide, into a framework of a homologous variable region human antibody. The resultant humanized heavy and light chain variable regions are then connected to each other via a suitable linker, wherein the linker should be flexible and of length that it allows heavy chain binding to light chain but discourages heavy chain of one molecule binding to the light chain of another. For example a linker of about 10-15 residues. Preferably, the linker includes [(Glycine)₄ (Serine)₁]₃ (SEQ ID NOS: 401-402), but is not limited to this sequence as other sequences are possible.

In one aspect, the humanized variable regions of MNE6 (SEQ ID NOS: 38-39 and 93-94), MNC2 (SEQ ID NOS: 144-145 and 194-195), or other antibodies of the invention are biochemically grafted into a construct that connects heavy and light chains via a linker. Examples of humanized single chain anti-MUC1* antibodies comprising humanized sequences from the variable regions of MNE6 and MNC2, were generated. Several humanized MNE6 single chain proteins were generated (SEQ ID NOS: 232-237, 397-398). Several humanized MNC2 single chain proteins were generated (SEQ ID NOS: 238-243, 395-396, 654-655, 5017-5018, 5019-5020). Several humanized 20A10 single chain proteins were generated (SEQ ID NOS:1576-1581 and 5001-5012). Several humanized 3C2B1 single chain proteins were generated (SEQ ID NOS:1812-1813). Several humanized 5C6F3 single chain proteins were generated (SEQ ID NOS: 1814-1815). Several humanized 25E6 single chain proteins were generated (SEQ ID NOS:1600-1601). In a preferred embodiment, humanized anti-MUC1* antibody fragments, including variable fragments, scFv antibody fragments derived from MNE6 scFv, MNC2 scFv, 20A10, 3C2B1, 5C6F3, 25E6, 18G12, 28F9, 1E4, B12, B2, B7, B9, 8C7F3, or H11 scFv, which may be incorporated into different therapeutic formats including CARs, Bispecific antibodies, BiTEs, antibody drug conjugates, are administered to a person diagnosed with or at risk of developing a MUC1-positive cancer.

One aspect of the invention is a method for treating a patient diagnosed with, suspected of having, or at risk of developing a MUC1 positive or MUC1* positive cancer, wherein the patient is administered an effective amount of an agent containing a monomeric form of MNE6, MNC2, or 20A10, 3C2B1, 5C6F3, 25E6, 18G12, 28F9, 1E4, B12, B2, B7, B9, 8C7F3, or H11, wherein the antibody variable fragment portions are human or have been humanized and wherein the Fc portion of the antibody-like protein, if present, has been mutated such that it resists dimer formation.

### CAR T and Cancer Immunotherapy Techniques

In another aspect of the invention, some or all of the single chain portions of anti-MUC1* antibody fragments are biochemically fused onto immune system molecules, using several different chimeric antigen receptor, ‘CAR’ strategies. The idea is to fuse the recognition portion of an antibody, typically as a single chain variable fragment, to an immune system molecule that has a transmembrane domain and a cytoplasmic tail that is able to transmit signals that activate the immune system for example activating the immune cell to kill the cell that is recognized by the recognition unit. The recognition unit can be an antibody fragment, a single chain variable fragment, scFv, or a peptide. In one aspect, the recognition portion of the extracellular domain of the CAR is comprised of sequences from the human, humanized or non-human variable regions of MNE6 (SEQ ID NOS:12-13 and 65-66, 56-57 and 107-108, 38-39 and 93-94, 341-342, 391-394), MNC2 (SEQ ID NOS:118-119 and 168-169, or 144-145 and 194-195, 654-655, 239-243, or 5017-5020), 20A10 (SEQ ID NOS:988-989 and 1004-1005, or 1574-1581, 1677, 1687 or 5001-5012), 3C2B1 (SEQ ID NOS:1386-1413, or 1820-1823, 1572-1573, or 1812-1813), 5C6F3 (SEQ ID NOS:1816-1819, or 1384-1385, or 1814-1815), 25E6 (SEQ ID NOS:1020-1021, or 1036-1037, or 1598-1601), 18G12, 28F9, 1E4, B12, B2, B7, B9, 8C7F3, or H11. These are examples of murine or humanized antibodies of the invention, or their single chain fragments, scFv's, which can be incorporated into CARs, BiTEs or ADCs. In another aspect, the recognition unit is comprised of sequences from a single chain variable fragment. Examples of single chain constructs are given. Several humanized MNE6 single chain proteins, scFv, were generated (SEQ ID NOS: 232-237). Several humanized MNC2 single chain proteins, scFv, were generated (SEQ ID NOS: 238-243, 654-655, or 5017-5020). Several humanized 20A10 single chain proteins, scFv, were generated (SEQ ID NOS:1576-1581, 1677, 1687 and 5001-5012). Humanized single chain proteins were also derived from 3C2B1 (SEQ ID NOS:1812-1813), 5C6F3 (SEQ ID NOS:1814-1815) and 25E6 (SEQ ID NOS:1600-1601). The extracellular hinge of the CAR can be derived from a variety of proteins, including CD8 (SEQ ID NOS:345-346), CD4 (SEQ ID NOS:347-348) or CD28 (SEQ ID NOS:349-350). The transmembrane region of the CAR can also be derived from CD3-zeta (SEQ ID NOS:361-362), CD8 (SEQ ID NOS:363-364), CD4 (SEQ ID NOS:365-366), CD28 (SEQ ID NOS:367-368), 4-1BB (SEQ ID NOS:369-370), OX40 (SEQ ID NOS:371-372), antibody domains or other transmembrane region, including the transmembrane region of the proximal cytoplasmic co-stimulatory domain, such as CD28, 4-1BB or other. The cytoplasmic tail of the CAR can be comprised of one or more motifs that signal immune system activation. A group of cytoplasmic signaling motifs, sometimes referred to as co-stimulatory domains, includes but is not limited to CD27, CD28 (SEQ ID NOS:377-378), 4-1BB (SEQ ID NOS:379-380), OX40, CD30, CD40, ICAm-1, LFA-1, ICOS, CD2, CD5, CD7 and Fc receptor gamma domain. The signaling domain can be CD3-zeta (SEQ ID NOS:373-374, or 375-376) or a modified CD3-zeta called 1XX (SEQ ID NOS:1796-1797). A minimal CAR may have the CD3-zeta or an Fc receptor gamma domain then one or two of the above domains in tandem on the cytoplasmic tail. In one aspect, the cytoplasmic tail comprises CD3-zeta, or a mutant thereof such as 1XX, plus a co-stimulatory domain such as CD28, 4-1BB and/or OX40. In another aspect, one or two ITAMs of CD3-zeta are deleted or mutated to slow signaling which increases persistence and decreases differentiation of the immune cell.

The extracellular domain recognition unit of a MUC1* targeting CAR can comprise variable regions of any non-human, humanized or human antibody that is able to bind to at least 12 contiguous amino acids of the PSMGFR peptide (SEQ ID NO:2) or more preferably the N-10 peptide (SEQ ID NO:3), still more preferably, is able to bind to the N-10 peptide (SEQ ID NO:3), but is not able to bind to the C-10 peptide (SEQ ID NO:825). In one aspect, the MUC1* targeting portion of the CAR comprises variable regions from non-human, humanized or human MNC2, MNE6, 20A10, 3C2B1, 5C6F3, 25E6, 18G12, 28F9, 1E4, B12, B2, B7, B9, 8C7F3, or H11. In a preferred embodiment, the MUC1* targeting portion of the CAR comprises variable regions from non-human, humanized or human MNC2, MNE6, 20A10, 3C2B1, 5C6F3, 25E6. Examples of a few antibodies of the invention, incorporated into CARs as either murine or humanized are given as MNE6 (SEQ ID NOS:297-298, 300-301, 303-304, 1626-1633 and 5045-5048), MNC2 (SEQ ID NOS:306-307, 608-611, 718-719, 1618-1625, 5041-5044, and 1784-1785), 20A10 (SEQ ID NOS:1582-1597, 5021-5028, 1798-1799, 1692,1699, and 1706), and 25E6 (SEQ ID NOS:1602-1617, 5033-5040). Similarly, single chain antibodies derived from 3C2B1 (SEQ ID NOS:1572-1573 or 1812-1813) or 5C6F3 (SEQ ID NOS:1384-1385 or 1814-1815) can be substituted for the single chain antibody fragment in any of the CARs listed above. In the humanization process, the antibody CDRs can be inserted into a number of different framework regions; as a demonstration we generated three versions of a humanized 20A10 which differ only in the framework regions. These have been incorporated into CARs (SEQ ID NOS:1675, 1678, 1685) that when transduced into human T cells are able to recognize target MUC1* expressing cells and kill them. In one aspect, the extracellular domain recognition unit of a CAR is comprised essentially of a humanized MNC2, MNE6, 20A10, 3C2B1, 5C6F3, 25E6, 18G12, 28F9, 1E4, B12, B2, B7, B9, 8C7F3, or H11 single chain variable fragment scFv. The recognition domain, which is typically an antibody fragment, can be fused to an extracellular region, often referred to as the hinge. The hinge can be derived from a variety of extracellular regions or peptides, including but not limited to the hinge region of CD8 (SEQ ID NOS:345-346), CD4 (SEQ ID NOS:347-348) or CD28 (SEQ ID NOS:349-350). The transmembrane region of the CAR can be derived from a number of protein transmembrane domains, including but not limited to CD8 (SEQ ID NOS:363-364), or can be the transmembrane domain of CD3-zeta (SEQ ID NOS:361-362), CD4 (SEQ ID NOS:365-366), CD28 (SEQ ID NOS:367-368), 41BB (SEQ ID NOS:369-370), OX40 (SEQ ID NOS:371-372) or other transmembrane region. The cytoplasmic domain of a CAR with antibody fragment targeting MUC1* extracellular domain can be comprised of one or more selected from the group comprising an immune system co-stimulatory cytoplasmic domain and a cytoplasmic signaling domain. The group of immune system co-stimulatory domains includes but is not limited to CD27, CD28, 4-1BB, OX40, CD30, CD40, ICAm-1, LFA-1, ICOS, CD2, CD5, CD7 and Fc receptor gamma domain (SEQ ID NOS:373-382). The group of immune system signaling domains includes but is not limited to CD3-zeta (SEQ ID NOS:373-376) and CD3-zeta-1XX (SEQ ID NOS:1796-1797). The CD3-zeta signaling domain may be wild type or may contain deletions or mutations of one or two of the three ITAMs. In one aspect, the CD3-zeta domain contains only one functional ITAM. In a preferred embodiment that ITAM is ITAM1 also known as the 1XX variation of CD3-zeta.

The CARs described can be transfected or transduced into a cell of the immune system. In a preferred embodiment, a MUC1* targeting CAR is transfected or transduced into a T cell or an NK cell. The immune cell can be autologous or allogeneic. In one aspect, the T cell is a CD3+ T cell, which may be CD8 or CD4 positive. In another case it is a dendritic cell. In another case it is a B cell. In another case it is a mast cell. In yet another case it is a Natural Killer, NK, cell. In another aspect, the immune cell is derived from a stem cell that has been directed to differentiate to that immune cell type in vitro. In another aspect, a CAR containing sequences of the antibody are expressed in a stem cell, which then may be differentiated into an immune cell. In one case, the immune cell is a T cell. In another case, the immune cell is an NK cell. The cell can be from a patient or from a donor. If from a donor, it can be engineered to remove molecules that would trigger rejection. Cells transfected or transduced with a CAR of the invention can be expanded ex vivo or in vitro then administered to a patient. Administrative routes are chosen from a group containing but not limited to bone marrow transplant, intravenous injection, in situ injection or transplant. In a preferred embodiment, the MUC1* targeting CAR is administered to a person diagnosed with or at risk of developing a MUC1-positive cancer.

There are many possible anti-MUC1* CAR constructs that can be transduced into T cells or other immune cells for the treatment or prevention of MUC1* positive cancers. CARs are made up of modules and the identity of some of the modules is relatively unimportant, while the identity of other modules is critically important. We and others have shown that intracellular signaling modules, such as CD3-zeta (SEQ ID NOS: 373-376), CD28 (SEQ ID NOS: 377-378) and 41BB (SEQ ID NOS: 379-380), alone or in combinations stimulate immune cell expansion, cytokine secretion and immune cell mediated killing of the targeted tumor cells (Pule M A, Straathof K C, Dotti G, Heslop H E, Rooney C M and Brenner M K (2005) A chimeric T cell antigen receptor that augments cytokine release and supports clonal expansion of primary human T cells. Mol Ther. 12(5):933-941; Hombach A A, Heiders J, Foppe M, Chmielewski M and Abken H. (2012) OX40 costimulation by a chimeric antigen receptor abrogates CD28 and IL-2 induced IL-10 secretion by redirected CD4(+) T cells. Oncoimmunology. 1(4):458-466; Kowolik C M, Topp M S, Gonzalez S, Pfeiffer T, Olivares S, Gonzalez N, Smith D D, Forman S J, Jensen M C and Cooper U. (2006) CD28 costimulation provided through a CD19-specific chimeric antigen receptor enhances in vivo persistence and antitumor efficacy of adoptively transferred T cells. Cancer Res. 66(22):10995-11004; Loskog A, Giandomenico V, Rossig C, Pule M, Dotti G and Brenner MK. (2006) Addition of the CD28 signaling domain to chimeric T cell receptors enhances chimeric T cell resistance to T regulatory cells. Leukemia. 20(10):1819-1828; Milone M C, Fish J D, Carpenito C, Carroll R G, Binder G K, Teachey D, Samanta M, Lakhal M, Gloss B, Danet-Desnoyers G, Campana D, Riley J L, Grupp S A and June C H. (2009) Chimeric receptors containing CD137 signal transduction domains mediate enhanced survival of T cells and increased antileukemic efficacy in vivo. Mol Ther. 17(8):1453-1464; Song D G, Ye Q, Carpenito C, Poussin M, Wang L P, Ji C, Figini M, June C H, Coukos G, Powell D J Jr. (2011) In vivo persistence, tumor localization, and antitumor activity of CAR-engineered T cells is enhanced by costimulatory signaling through CD137 (4-1BB). Cancer Res. 71(13):4617-4627). Antibodies of the invention including but not limited to fragments of MNC2, MNE6, 20A10, 3C2B1, 5C6F3, 25E6, 18G12, 28F9, 1E4, B12, B2, B7, B9, 8C7F3, or H11 can also be incorporated into CARs that have mutated cytoplasmic tails, such as mutated or deleted tyrosines of one or more of the ITAMs. In any of the CARs described above, the cytoplasmic tails may include mutations or deletions that dampen signaling, which increases persistence and decreases host cell differentiation. Such mutations include but are not limited to Tyrosines that are mutated to inhibit phosphorylation and signaling (Salter et al, 2018;). In any of the CARs described above, the ITAMs of CD3-zeta may be mutated to inhibit or dampen signaling (Feucht et al 2019). In any of the CARs described above, the CD3-zeta of the cytoplasmic tail may comprise mutations or deletions in the ITAMs including those referred to as 1XX (SEQ ID NOS:1796-1797). In another aspect one or two ITAMs are deleted, leaving only one or two ITAMs (Feucht et al 2019). In another aspect, the position of the included ITAM or ITAMs is moved to a position proximal to the co-stimulatory domain. Suitable ITAM configurations for increased persistence of CARs include but are not limited to 1XX, X2X, XX3, 12X and 23X, wherein the numeral 1, 2 or 3 refers to ITAM1, ITAM2, or ITAM3 and X refers to the deletion of that ITAM. In a preferred embodiment ITAM1 is the only functional ITAM included in the CAR construct, also known as 1XX. Examples of antibodies of the invention incorporated into CARs with 1XX mutations in ITAMs of CD3-zeta are given in the following sequences: MNC2 (SEQ ID NOS: 1618-1625, 5041-5044 and 1784-1785), MNE6 (SEQ ID NOS:1626-1633 and 5045-5048), 20A10 (SEQ ID NOS:1590-1597, 5021-5028, and 1798-1799), 25E6 (SEQ ID NOS:1610-1617 and 5037-5040). The transmembrane and extracellular hinge region of the CAR may or may not be derived from sequences of the adjacent co-stimulatory domain. For example, a CAR comprising the 4-1BB co-stimulatory domain may have a transmembrane and hinge region derived from CD8 or CD28. In another example, a CAR comprising the CD28 co-stimulatory domain may have a transmembrane and hinge region derived from CD28. Examples of antibodies of the invention incorporated into CARs with 1XX mutations in ITAMs of CD3-zeta, which have a CD28 co-stimulatory domain as well as transmembrane and hinge region derived from CD28, are given in the following sequences: MNC2 (SEQ ID NOS:5041-5044 and 1784-1785) MNE6 (SEQ ID NOS:5045-5048), 20A10 (SEQ ID NOS:5025-5028, 1798-1799, 1692,1699, and 1706); 25E6 (SEQ ID NOS:5037-5040). In any of the CARs described here, the cytoplasmic region may be comprised of one or more of signaling sequence motifs and co-stimulatory domains, including but not limited to CD3-zeta, CD3-zeta-1XX, CD27, CD28, 4-1BB, OX40, CD30, CD40, ICAm-1, LFA-1, ICOS, CD2, CD5, or CD7. Additionally, the sequence of the intracellular signaling domain may contain mutations, such as CD3-zeta-1XX (SEQ ID NOS:1796-1797) that dampen the signal to improve persistence or target cell killing. Signaling domain CD3-zeta may be wild type or may contain mutations or deletions of one or two ITAMs. In a preferred embodiment, ITAMs 2 and 3 are deleted or inactivated, leaving a single ITAM, which is ITAM1 also known as the 1XX construct.

In one aspect of the invention, the hinge and transmembrane regions of CAR are derived from CD8 (SEQ ID NO: 301, 719, 1675 or 1605). In another aspect of the invention, the hinge and transmembrane regions of CAR are derived from CD28 (SEQ ID NO:5048, 5044, 5024 or 5036). In one aspect of the invention, the co-stimulatory domain is CD28 (SEQ ID NO: 298, 609, 1589, 1609). In another aspect of the invention, the co-stimulatory domain is 4-1BB (SEQ ID NO: 301, 719, 1585 or 1605). In a preferred embodiment, the antibody fragment that is the targeting head of the CAR, binds to the extracellular domain of a MUC1 that is devoid of the tandem repeat domain. In a more preferred embodiment, the antibody fragment that is the targeting head of the CAR, binds to a region of the MUC1* extracellular domain that contains the 35 most membrane proximal amino acids, also referred to here as N-10 (SEQ ID NO:3). In a still more preferred embodiment, the antibody fragment that is the targeting head of the CAR, binds to N-10 (SEQ ID NO:3) but does not bind to C-10 (SEQ ID NO:825). In a yet more preferred embodiment, the antibody fragment that is the targeting head of the CAR, binds to N-10 (SEQ ID NO:3), does not bind to C-10 (SEQ ID NO:825) and either does not bind to a linear epitope, that is to say doesn't work in a standard Western blot, or competes with NME7_{AB} for binding to the N-10 peptide (SEQ ID NO:3). In the CARs described here, the extracellular domain may include a murine, camelid, human, non-human or humanized single chain antibody fragment with framework region IV of the light chain having variable lengths as set forth as MNE6 scFv (SEQ ID NOS:5014 or 5016), MNC2 scFv (SEQ ID NOS:5018 or 5020) or 20A10 scFv (SEQ ID NOS 5002, 5004, 5006, 5008, 5010 or 5012), 25E6 scFv (SEQ ID NOS: 5030 or 5032). In any of the CARs described above, the Framework region IV of the light chain of the single chain antibody fragment may have the terminal amino acids R and T deleted or just T deleted. We note that the CDRs of antibodies can be inserted into a background of a number of different framework regions. As an example, 20A10 CDRs were inserted into three different sets of framework regions (SEQ ID NOS:1692, 1699 and 1706) and all were able to function when transduced into T cells. In any of the CARs described above, the T cell may be engineered to overexpress c-Jun as a method to inhibit T cell exhaustion (Lynn et al 2019). A variety of promoters can be used upstream of the genes for CARs and other compositions of the invention, including insertion into a naturally occurring promoter in the cell, such as the TRAC locus, using CRISPR, Sleeping Beauty or similar technology for site directed insertion of a gene. Among the promoters commonly used are the CMV promoter, or a mini CMV (SEQ ID NO: 1634), a minimal IL-2 promoter (SEQ ID NO: 1635), or Minimal Promoter minip (SEQ ID NO: 1636).

Single chain antibody fragments that included the variable domain of the monoclonal anti-MUC1* antibodies called MNE6 or MNC2 were engineered into a panel of CARs. The MUC1* targeting CARs were then transduced, separately or in combinations, into immune cells. When challenged with surfaces presenting a MUC1* peptide, an antigen presenting cell transfected with MUC1*, or MUC1* positive cancer cells, the immune cells that were transduced with MUC1* targeting CARs elicited immune responses, including cytokine release, killing of the targeted cells and expansion of the immune cells.

For example, the gene encoding the CARs and activated T cell induced genes described herein can be virally transduced into an immune cell using viruses, or inserted into a region downstream of one of the cell's promoters or enhancers, such as the TRAC (T cell receptor alpha chain) locus. Virus delivery systems and viral vectors including but not limited to retroviruses, including gamma-retroviruses, lentivirus, adenoviruses, adeno-associated viruses, baculoviruses, poxvirus, herpes simplex viruses, oncolytic viruses, HF10, T-Vec and the like can be used. In addition to viral transduction, CARs and activated T cell induced genes described herein can be directly spliced into the genome of the recipient cell using methods such as CRISPR technology, CRISPR-Cas9 and -CPF1, TALEN, Sleeping Beauty transposon system, and SB 100×.

Similarly, the identity of molecules that make up the non-targeting portions of the CAR, except for the CD3-zeta identity, such as the extracellular domain, transmembrane domain and membrane proximal portion of the cytoplasmic domain, are not essential to the function of a MUC1*-targeting CAR. For example, the extracellular domain, transmembrane domain and membrane proximal portion of the cytoplasmic domain can be comprised of portions of CD8, CD4, CD28, or generic antibody domains such as Fc, CH2CH3, or CH3. Further, the non-targeting portions of a CAR can be a composite of portions of one or more of these molecules or other family members. However, the identity of CD3-zeta is critical, as mutations, such as those referred to as 1XX or CD3-zeta-1XX, greatly affect in vivo persistence of CAR T cells. CAR T cells that express CARs whose cytoplasmic tail includes CD3-zeta-1XX have prolonged activity in vivo because they do not get exhausted as quickly as cells containing wild-type CD3-zeta. In addition, we have discovered that CARs with the 1XX signaling domain are more effective against cells characterized by low antigen density. Cancer cells with low antigen density may comprise a sub-population of a heterogeneous tumor. Cancer cells with low antigen density may be characteristic of early cancer cells that can lead to cancer recurrence. Additionally, tumors at the time of treatment may be comprised of cancer cells that express low levels of a particular cancer antigen.

Thus, in one embodiment, patients diagnosed with a cancer or at risk of developing a cancer or a cancer recurrence are treated with immune cells that express a CAR comprising a 1XX signaling domain. In one aspect, the patient is diagnosed with or at risk of developing a MUC1* cancer. In a preferred embodiment the recognition unit of the CAR comprises an antibody fragment that binds to the N-10 peptide (SEQ ID NO:3) but does not bind to the C-10 peptide (SEQ ID NO:825). In a more preferred embodiment, the antibody fragment is derived from MNC2, MNE6, 20A10, 3C2B1, 5C6F3, or 25E6.

In another embodiment, a patient diagnosed with a cancer comprised of tumor cells that express low levels of a targeted antigen, or diagnosed with an early cancer, or a patient who has been treated but still has residual tumor cells and is at risk of a cancer recurrence is treated with immune cells that express a CAR comprising a 1XX signaling domain, which enables the CAR T cells to kill both high and low antigen density cancer cells. In one aspect, the patient is diagnosed with or at risk of developing a MUC1* cancer. In a preferred embodiment the recognition unit of the CAR comprises an antibody fragment that binds to the N-10 peptide (SEQ ID NO:3) but does not bind to the C-10 peptide (SEQ ID NO:825). In a more preferred embodiment, the antibody fragment is derived from MNC2, MNE6, 20A10, 3C2B1, 5C6F3, or 25E6.

In yet another aspect of the invention, a patient diagnosed with a cancer or at risk of developing a cancer or a cancer recurrence is treated with an immune cell expressing a CAR with a wild-type CD3-zeta and is also treated with an immune cell expressing a CAR with a mutated CD3-zeta, such as CD3-zeta-1XX. In this way, the tumor is attacked by an immune cell expressing a CAR with full CD3-zeta signaling that efficiently kills off the high antigen expressing cells, but which are prematurely exhausted, while the cells expressing a CAR with a mutated CD3-zeta, such as CD3-zeta-1XX, persist longer in the patient and kill of the low antigen expressing cells that likely give rise to tumor recurrence. In one aspect, the patient is diagnosed with or at risk of developing a MUC1* cancer. In a preferred embodiment the recognition unit of the CAR comprises an antibody fragment that binds to the N-10 peptide (SEQ ID NO:3) but does not bind to the C-10 peptide (SEQ ID NO:825). In a more preferred embodiment, the antibody fragment is derived from MNC2, MNE6, 20A10, 3C2B1, 5C6F3, or 25E6. In one aspect, a patient is treated with a CAR T cell in which the CAR has a wild type CD3-zeta signaling domain, wherein the CAR is chosen from among the group comprising MNC2 CARs (SEQ ID NO:306-307, 608-611, 718-719), MNE6 CARs (SEQ ID NO:297-298, 300-301, 303-304), 20A10 CARs (SEQ ID NO:1582-1589, 5021-5024), 25E6 CARs (1602-1609, 5033-5036), a CAR comprising an antibody fragment derived from 3C2B1 wherein the signaling domain is wild type CD3-zeta, and a CAR comprising an antibody fragment derived from 5C6F3 wherein the signaling domain is wild type CD3-zeta. In another aspect, a patient is treated with a CAR T cell in which the CAR has a CD3-zeta-1XX signaling domain, wherein the CAR is chosen from among the group comprising MNC2 CARs (SEQ ID NO:1618-1625, 5041-5044, 1784-1785), MNE6 CARs (SEQ ID NO:1626-1633, 5045-5048), 20A10 CARs (SEQ ID NO:1590-1597, 5025-5028, 1798-1799), 25E6 CARs (SEQ ID NOS:1610-1617, 5037-5040), a CAR comprising an antibody fragment derived from 3C2B1 wherein the signaling domain is CD3-zeta-1XX, and a CAR comprising an antibody fragment derived from 5C6F3 wherein the signaling domain is CD3-zeta-1XX. In yet another aspect, a patient is treated with immune cells that express both a CAR having a wild type CD3-zeta signaling domain and a CAR having a 1XX signaling domain.

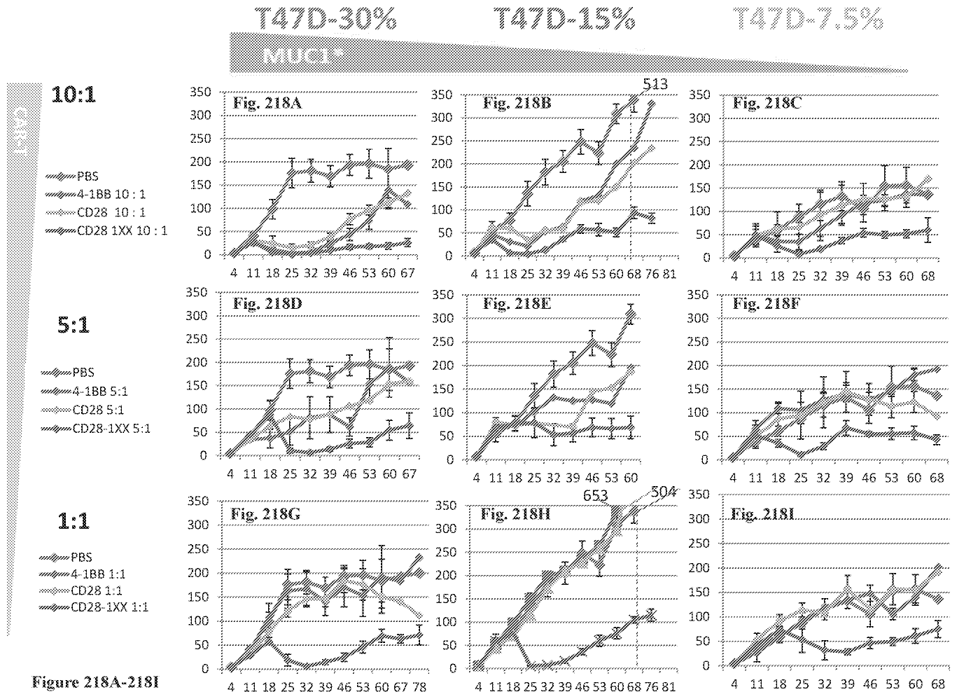

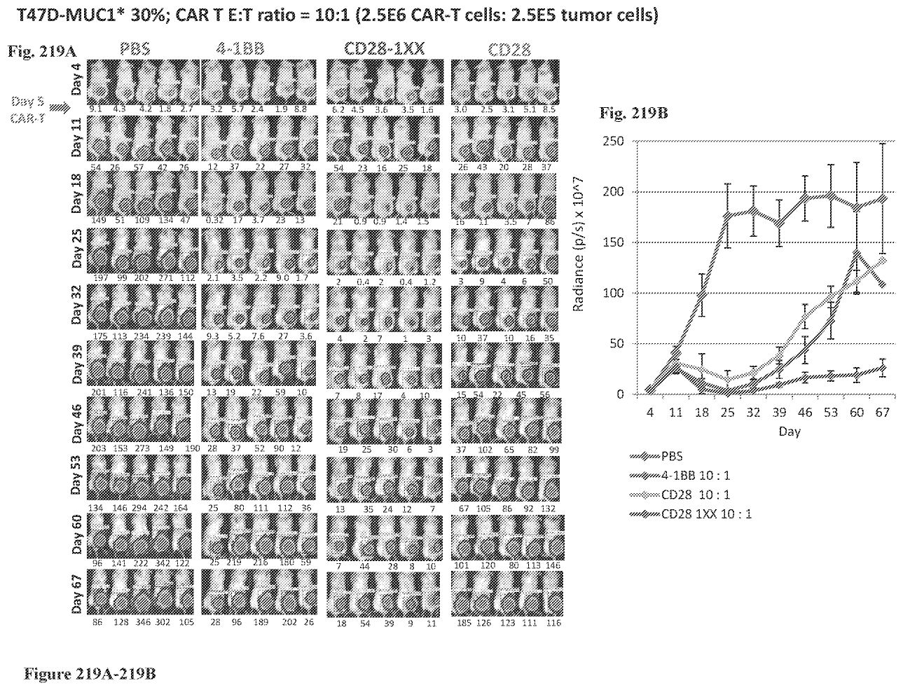

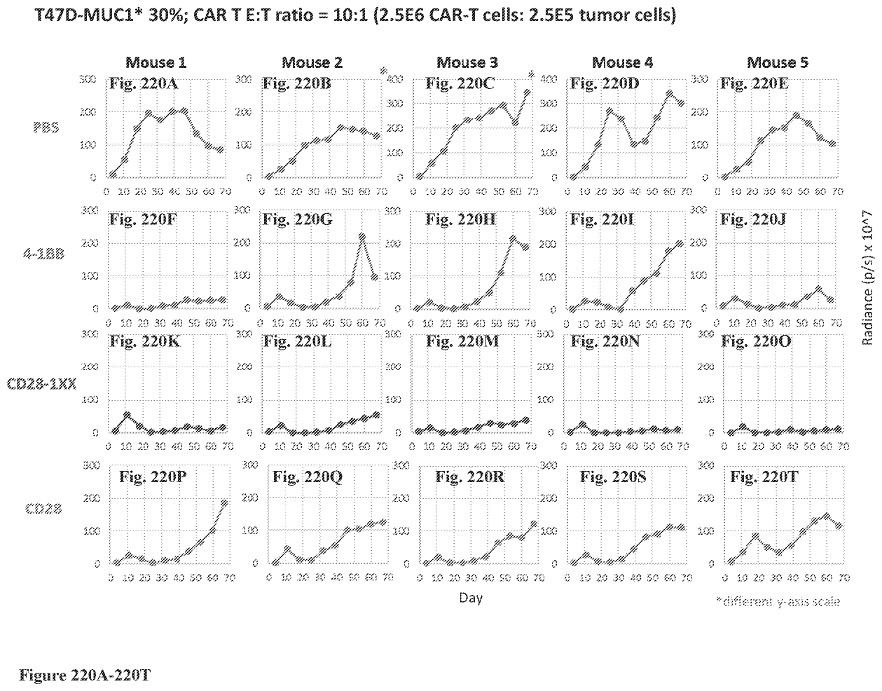

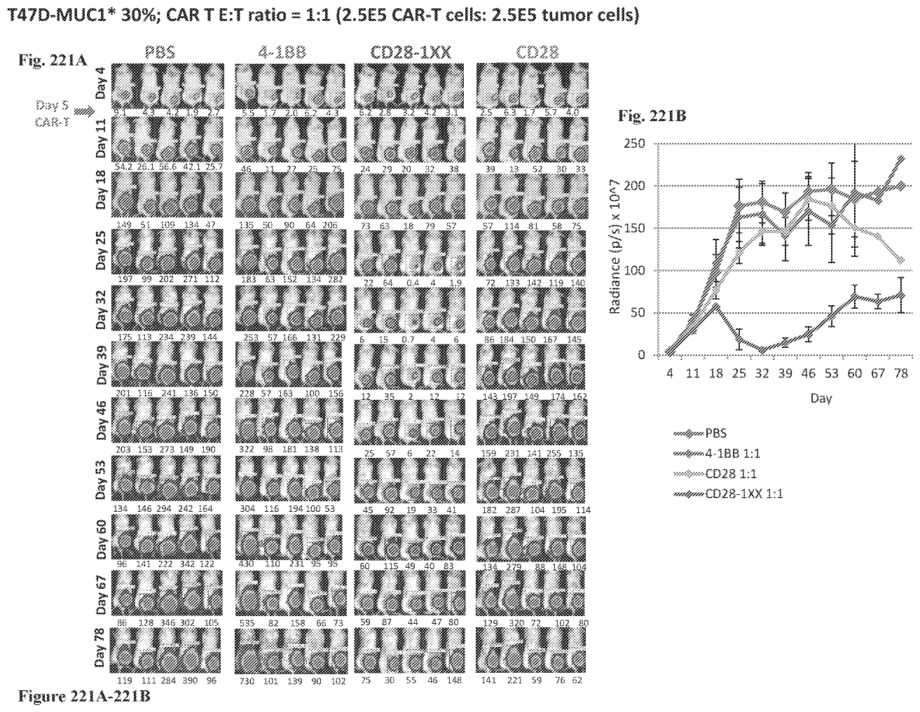

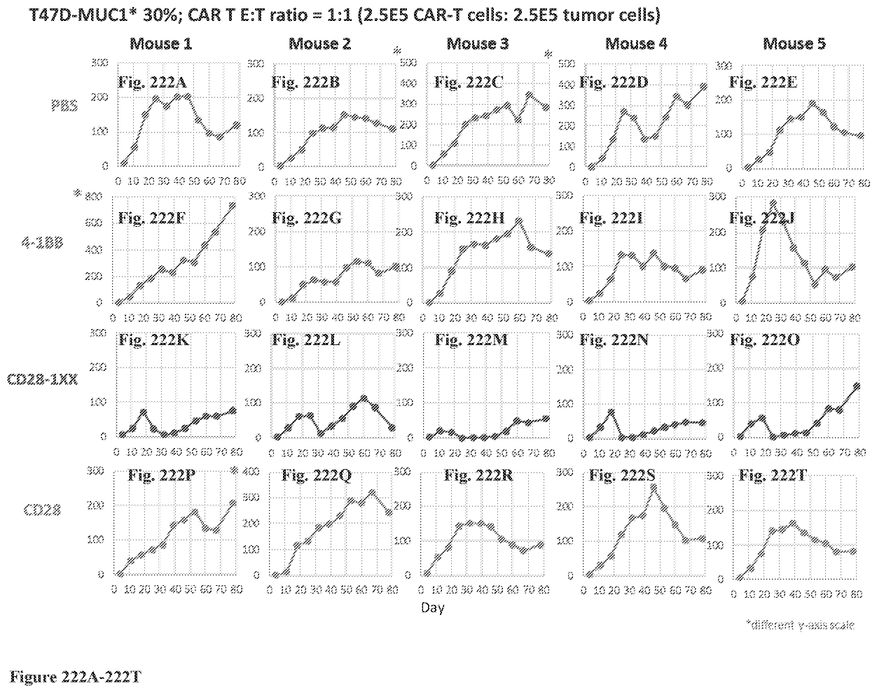

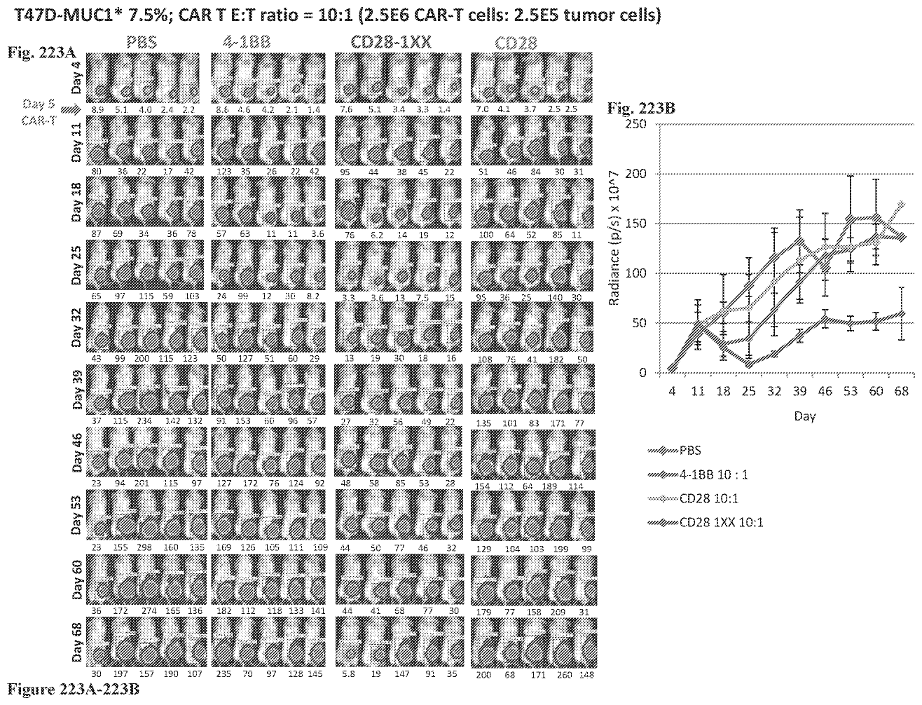

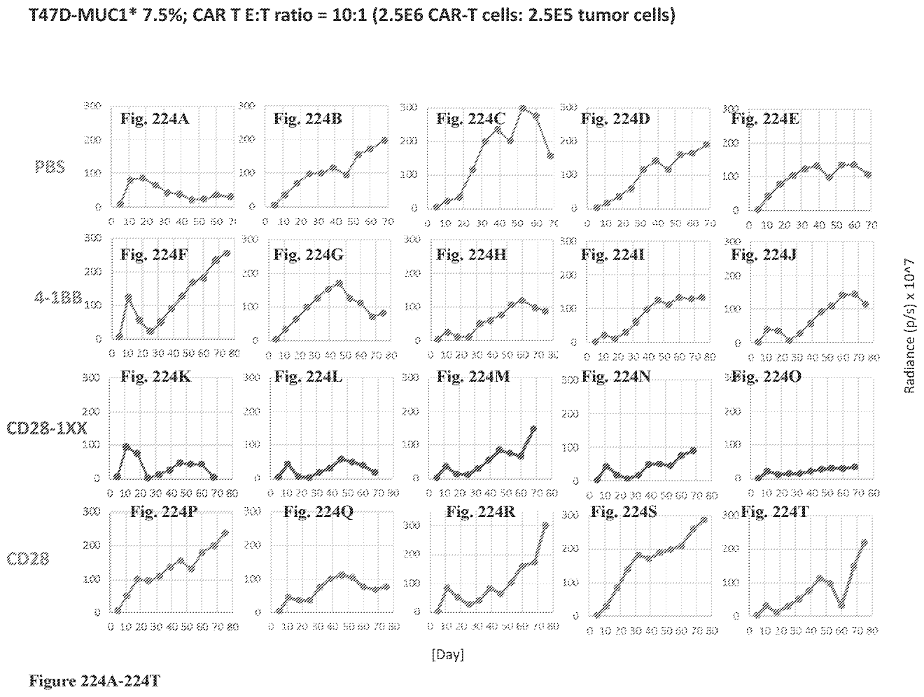

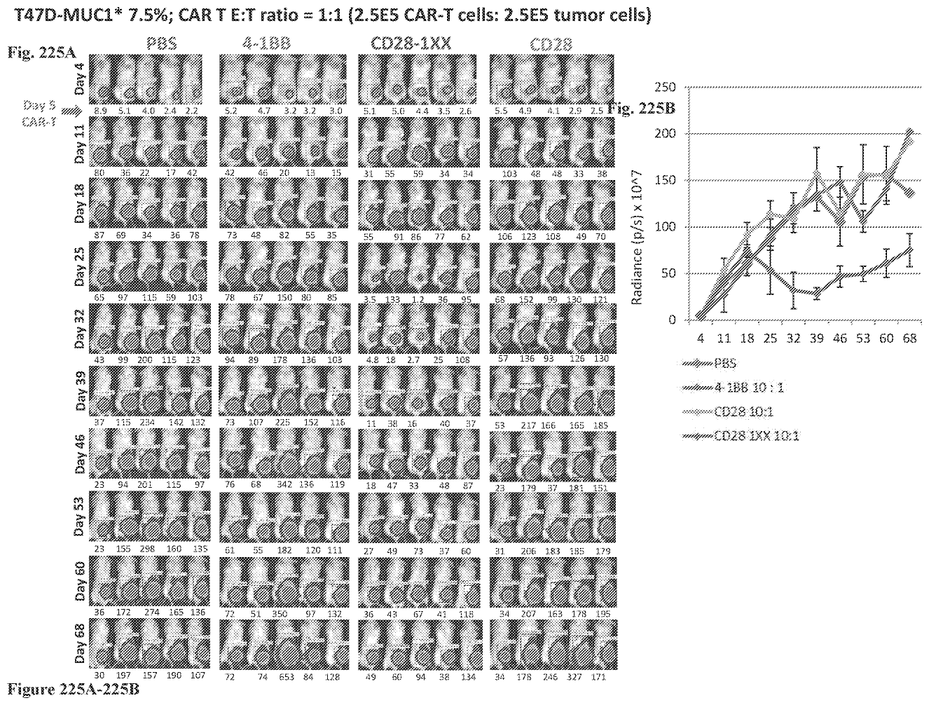

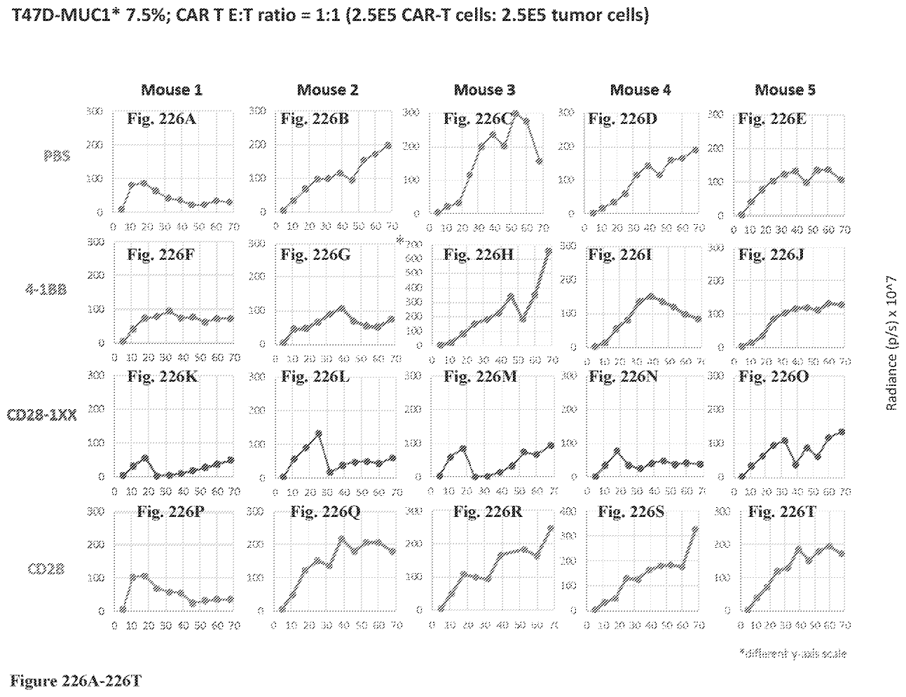

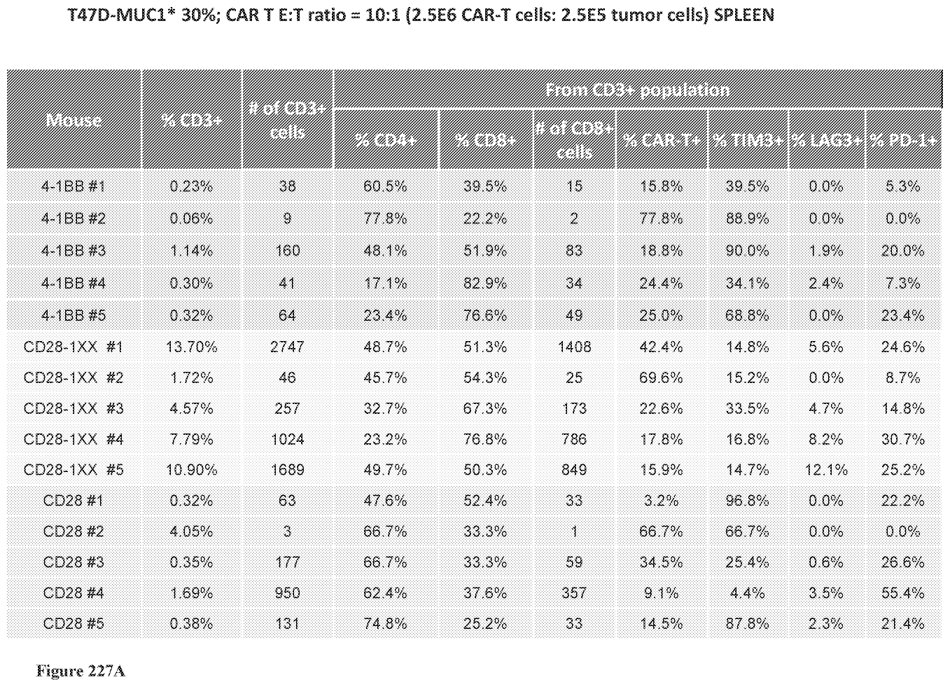

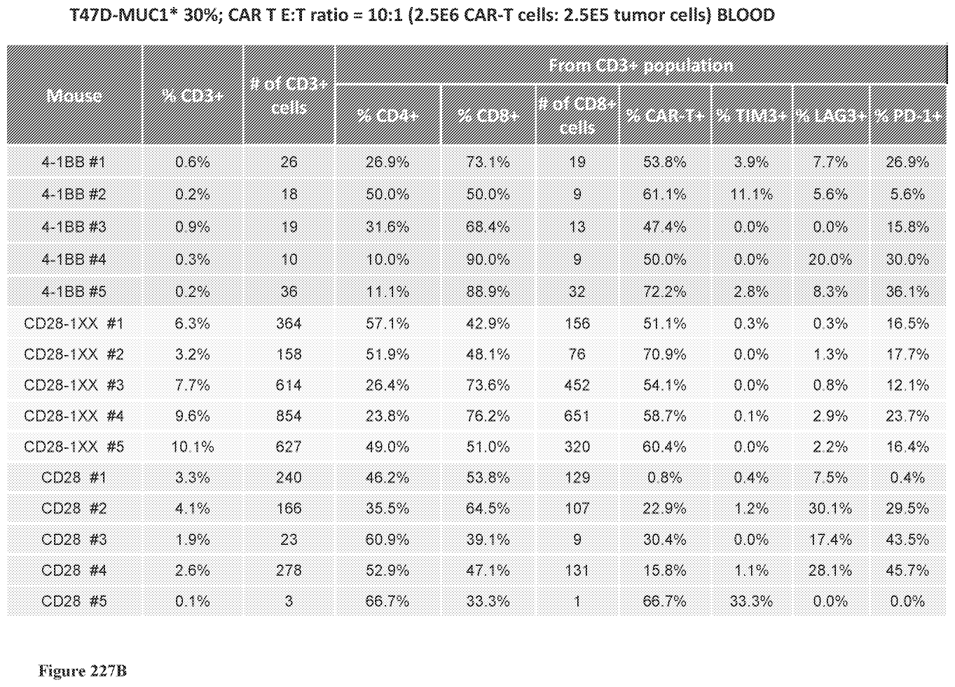

We have shown that CAR T cells bearing the 1XX mutations in the CD3-zeta are more effective than CAR T cells with wild type CD3-zeta at preventing tumor recurrence. FIG. 218 shows graphs of tumor volume measured by an IVIS instrument wherein the tumor cells have been genetically modified to express Luciferase. The substrate Luciferin was injected 10 minutes before the photo emissions were measured in the sedated animal. On Day 1 of the experiment, animals were injected sub-cutaneously with 250,000 human breast tumor cells. Tumors were made heterogeneous, comprised of two different tumor cell types. A first tumor cell population was T47D-wt, a breast cancer cell line that expresses both full-length MUC1 and the growth factor receptor form MUC1*, which we engineered to express mCherry fluorescence. The second tumor cell population was the same T47D breast cancer cells, except that they had been stably transduced to express even more MUC1* and GFP fluorescence, referred to here as T47D-MUC1*. In this experiment, animals were implanted with T47D-wt plus T47D-MUC1*, wherein the population of T47D-MUC1* made up 30%, 15% or 7.5% of the tumor population. Animals were then administered a one-time injection of either PBS, huMNC2-41BB-3z CAR T cells (4-1BB), huMNC2-CD28-3z CAR T cells (CD28) or huMNC2-CD28-1XX CAR T cells (CD28-1XX or 1XX). The CAR T cells were injected into the tail vein at an Effector to Target ratio (E:T) of 10:1, 5:1, or 1:1. As can be seen in these graphs, huMNC2-CD28-1XX out-performs huMNC2-CD28-3z and huMNC2-41BB-3z and the difference is more pronounced in tumors with low antigen density and when treatment is at a low CAR T cell dose. In follow-on experiments described here, it is clear that 1XX CAR T cells not only persist longer in vivo and stave off T cell exhaustion, but they also are much more effective at killing low antigen density tumor cells, which is a recognized problem in the industry and will lead to cancer recurrence. FIG. 219A-219B shows IVIS photographs and graphs of IVIS tumor volume measurement. FIG. 219A shows photographs of the mice that had been implanted with tumors in which 30% of the cancer cell population was T47D-MUC1*, referred to here as high antigen expressing cells. The various CAR T cells were administered at a CAR T to tumor cell ratio of 10:1 wherein 250,000 tumor cells were implanted and the animals were injected 5 days later with 2,500,000 CAR T cells. FIG. 219B shows a graph of the tumor volume by IVIS measurement by day. As can be seen, animals injected with the huMNC2-CD28-1XX had much smaller tumors than the animals treated huMNC2-4-1BB-3z or huMNC2-CD28-3z, which is the same CAR T except without the 1XX mutations in the CD3-zeta domain. FIGS. 220A-220T shows the IVIS graphs for 30% tumors treated at a CAR T to Tumor ratio of 10:1. Here graphs are shown for each individual animal rather than the average of the treatment group. FIGS. 221A-221B and FIGS. 222A-222T show essentially the same experiment except that the CAR T cell dose is lower with only 250,000 CAR T cells administered in a single injection. As can be seen, animals injected with the huMNC2-CD28-1XX had much smaller tumors than the animals treated huMNC2-4-1BB-3z or huMNC2-CD28-3z, which is the same CAR T except without the 1XX mutations in the CD3-zeta domain. However, with the lower dose of CAR T cells, even tumors in the huMNC2-CD28-1XX treated group begin to grow again, albeit more slowly.

In these next experiments (FIG. 223-FIG. 226) we show that in animals implanted with tumors in which only 7.5% of the tumor expresses high antigen density, it is even harder for standard CAR T cells to kill of the tumor. However, the same CAR T cells with the 1XX mutations in CD3-zeta are still quite effective at killing the tumor and inhibiting tumor recurrence. At a moderate CAR T cell dose having an effector to target ratio of 10:1, huMNC2-41BB-3z and huMNC2-CD28-3z show some long-term killing, which is exceeded by huMNC2-CD28-1XX. However, with low antigen density, which is a mimic of early cancer cells and residual, resistant cancer cells, and a low CAR T cell dose, having an effector to target ratio of 1:1, huMNC2-41BB-3z and huMNC2-CD28-3z did no better than the control PBS, whereas huMNC2-CD28-1XX continued to kill the tumor.

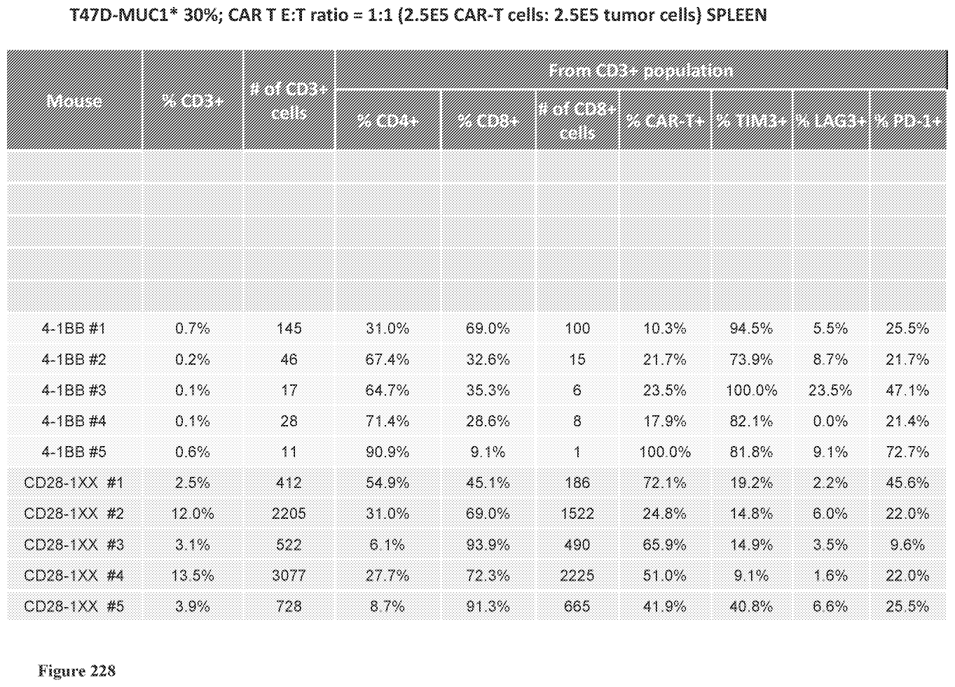

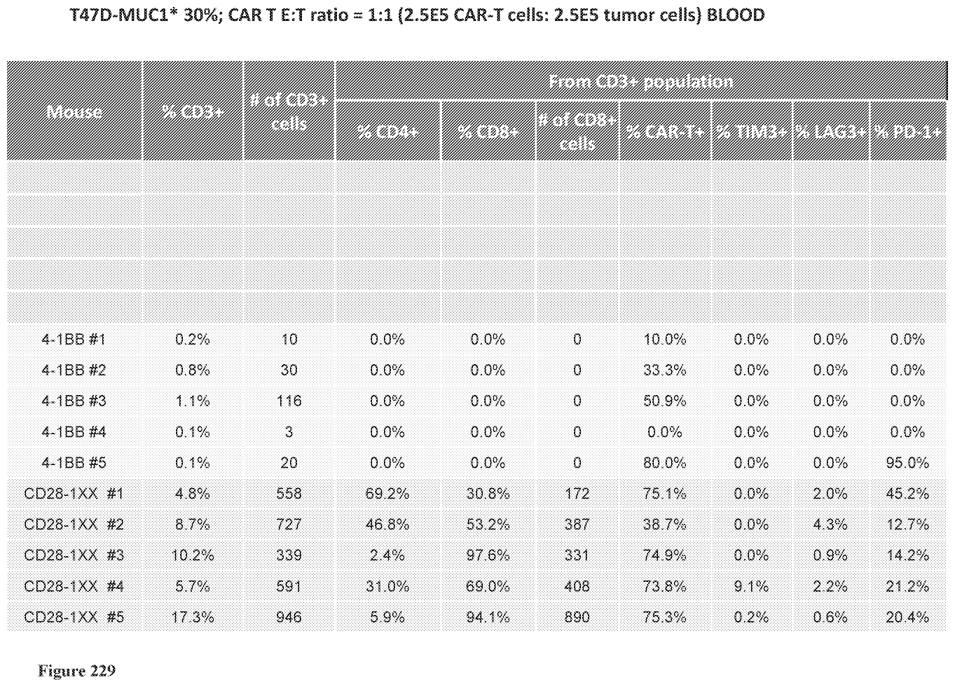

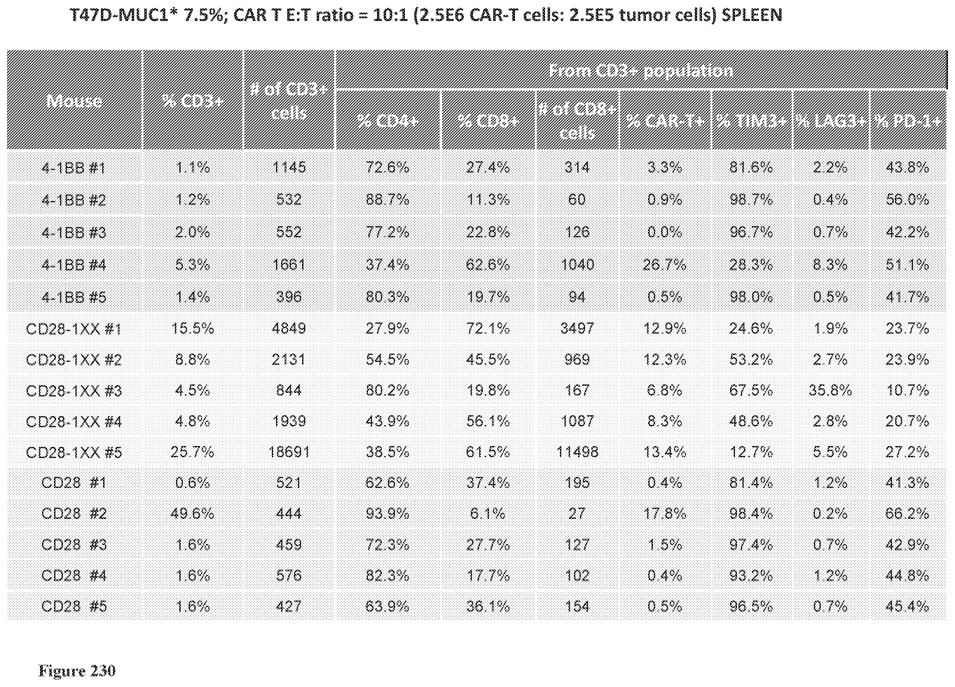

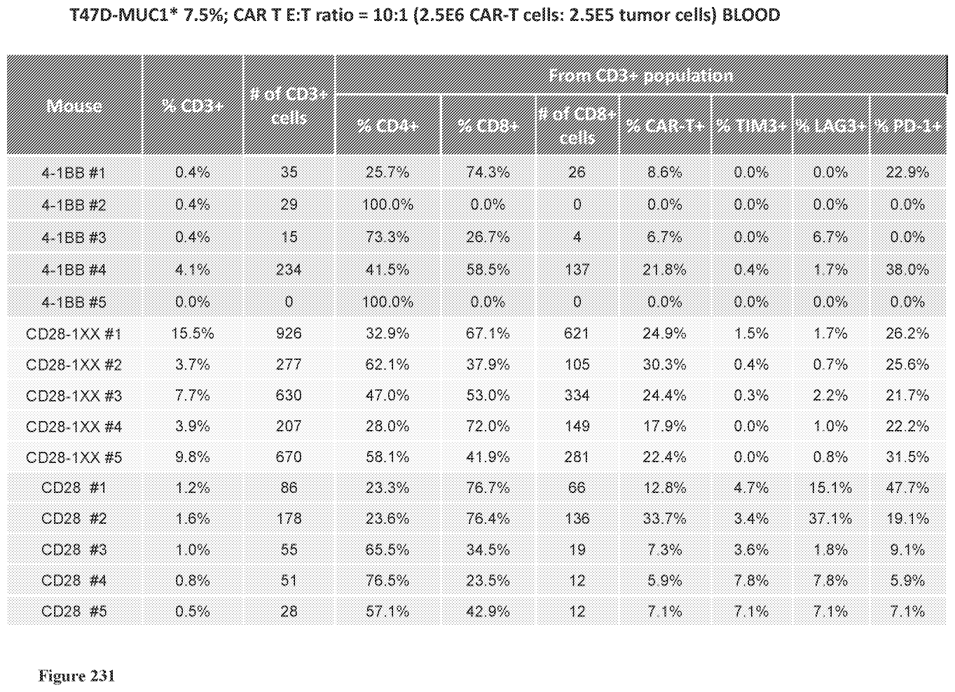

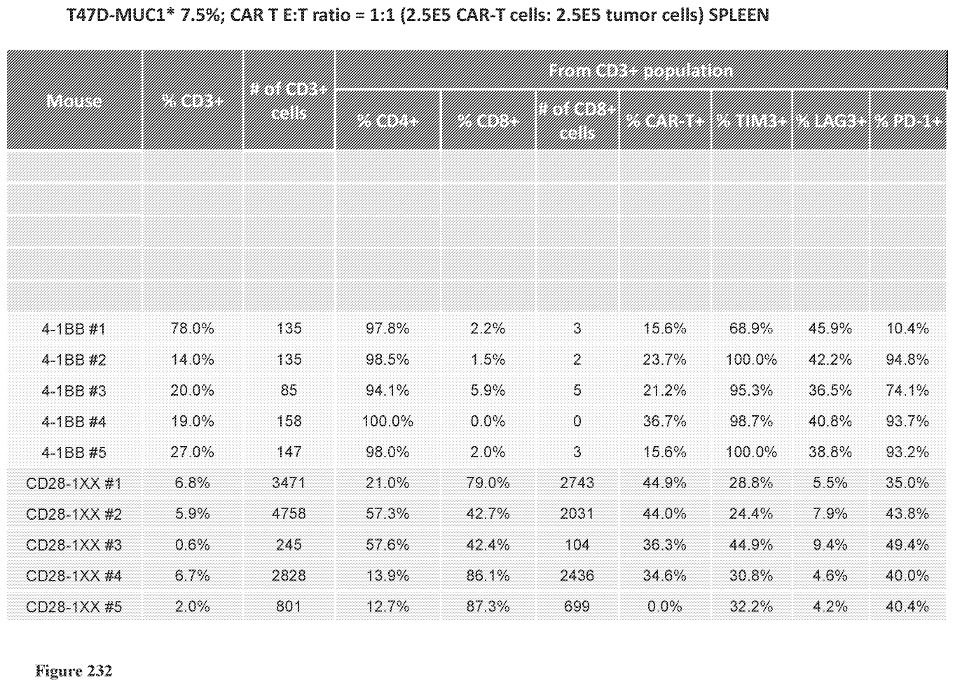

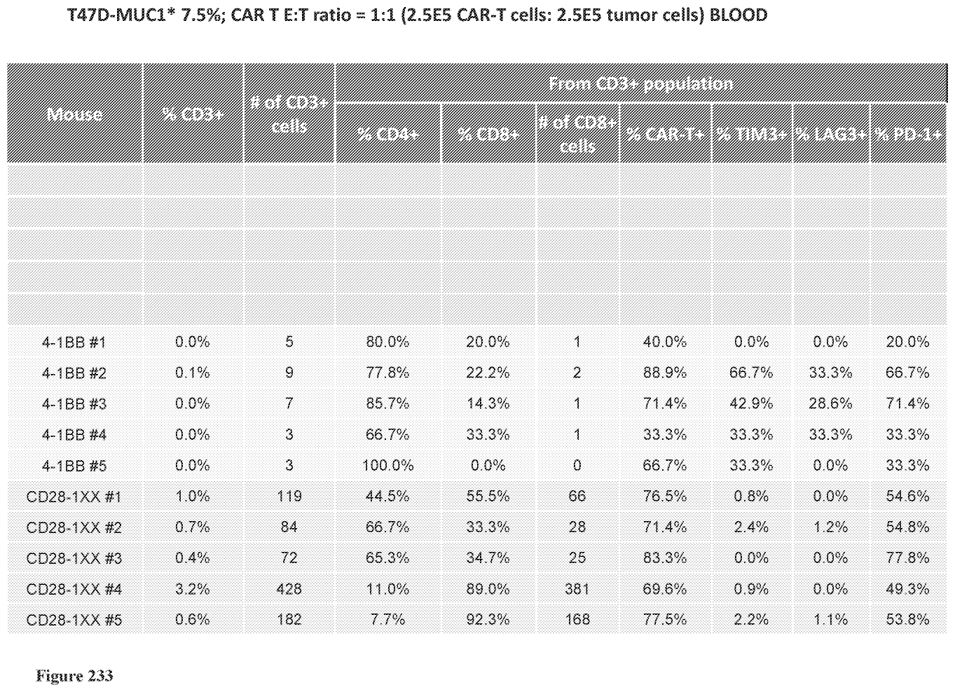

Animals were sacrificed between Day 69 and Day 90. FIGS. 227-233 show the tabulation of CD3 positive human T cells that were harvested from the spleens and from the blood of the test animals post sacrifice. As can be seen, the huMNC2-CD28-1XX treated mice, that had smaller tumors, have a greater number of live, persisting CAR T cells and CD8 positive killer T cells than the animals treated with the CAR T cells with wild type CD3-zeta. It is also seen that the molecular markers of T cell exhaustion, TIM3, LAG3 and PD-1, are greatly reduced or absent in the cells retrieved from the animals treated with huMNC2-CD28-1XX CAR T cells. This result is consistent with the idea that the 1XX mutations in CD3-zeta increase CAR T cell persistence in vivo.

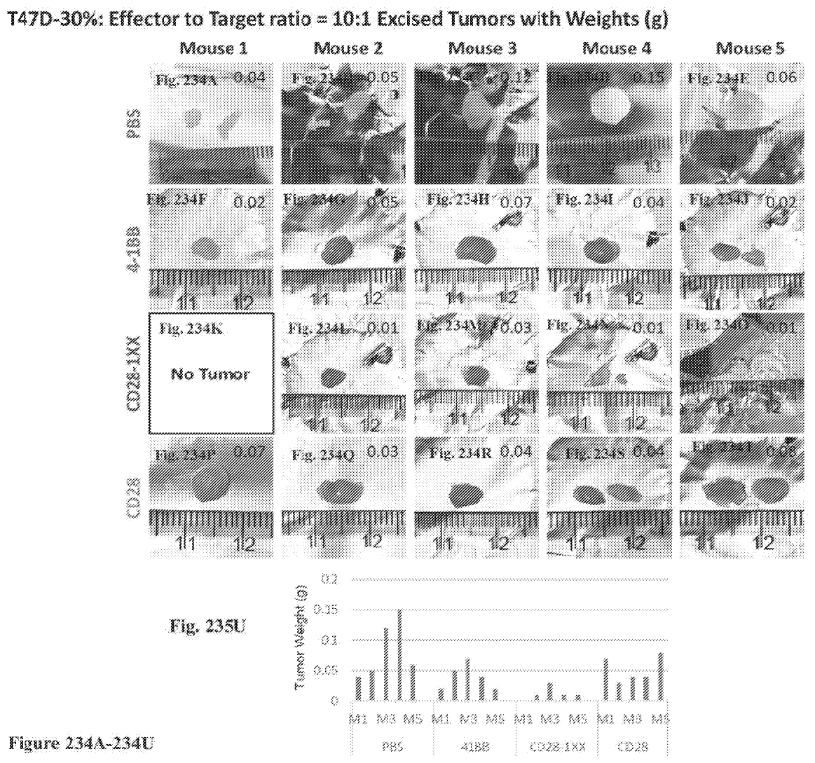

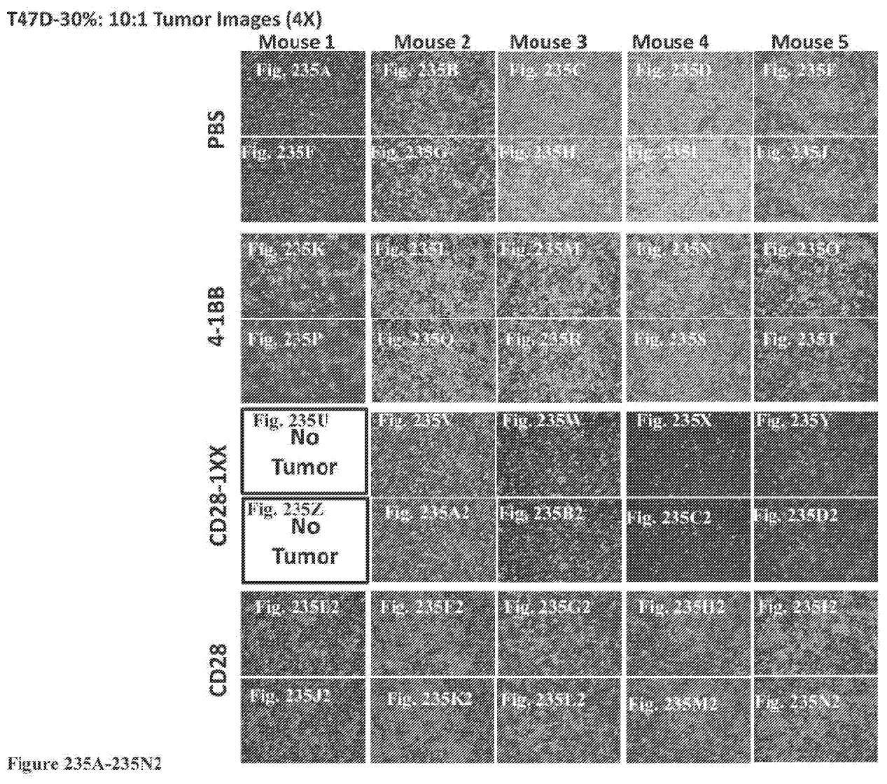

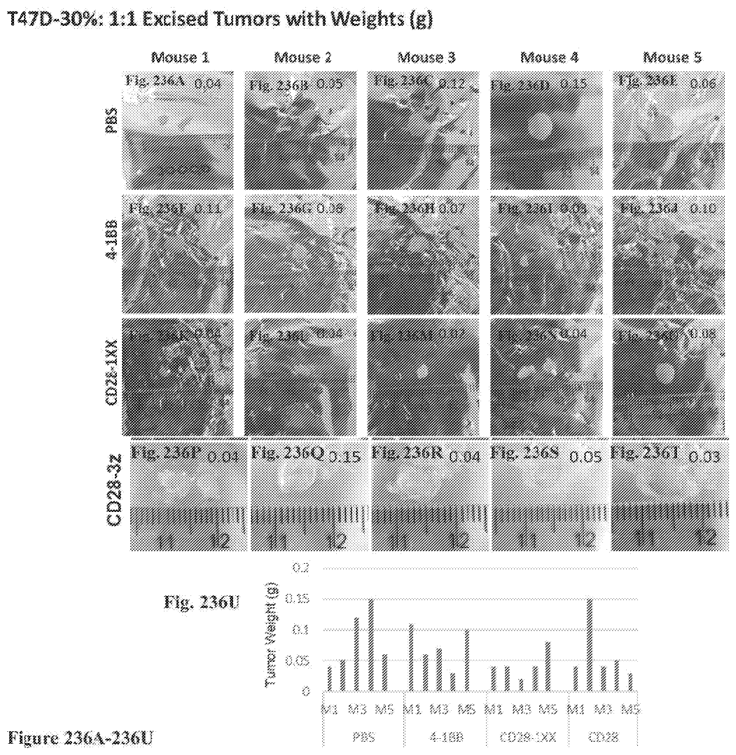

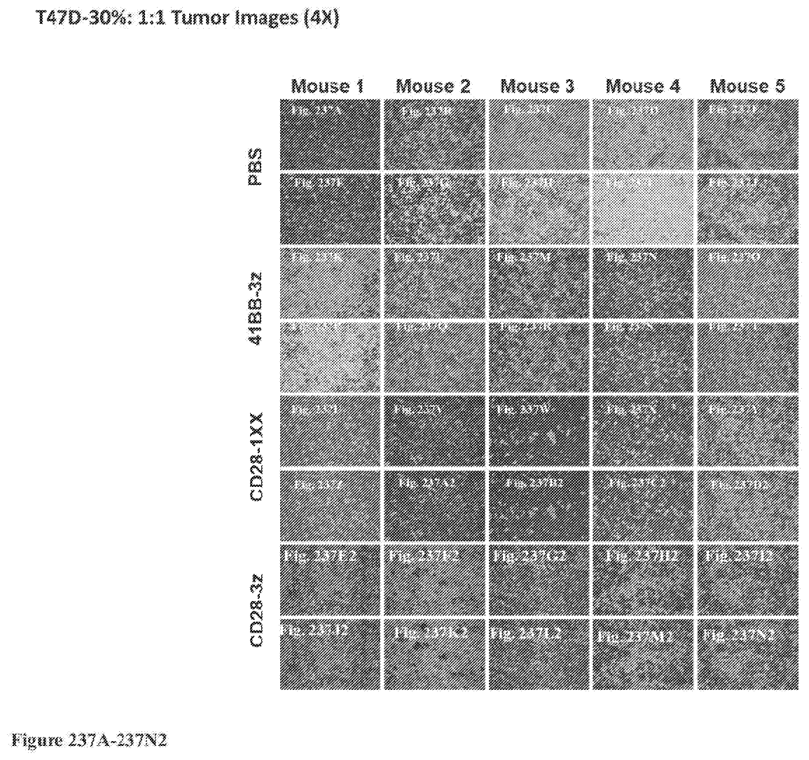

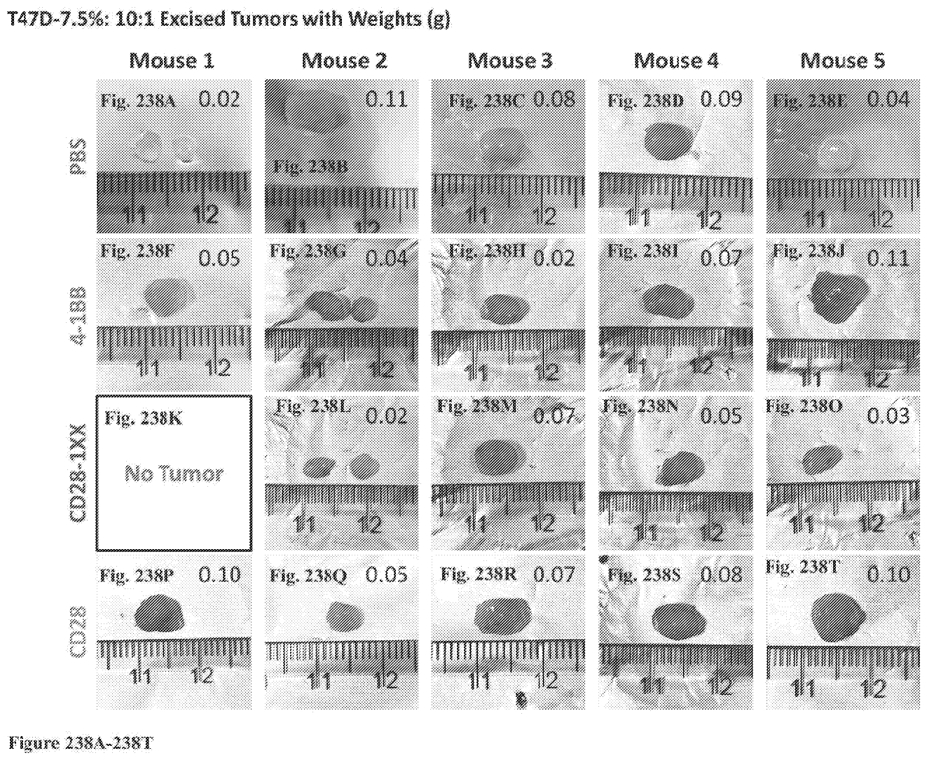

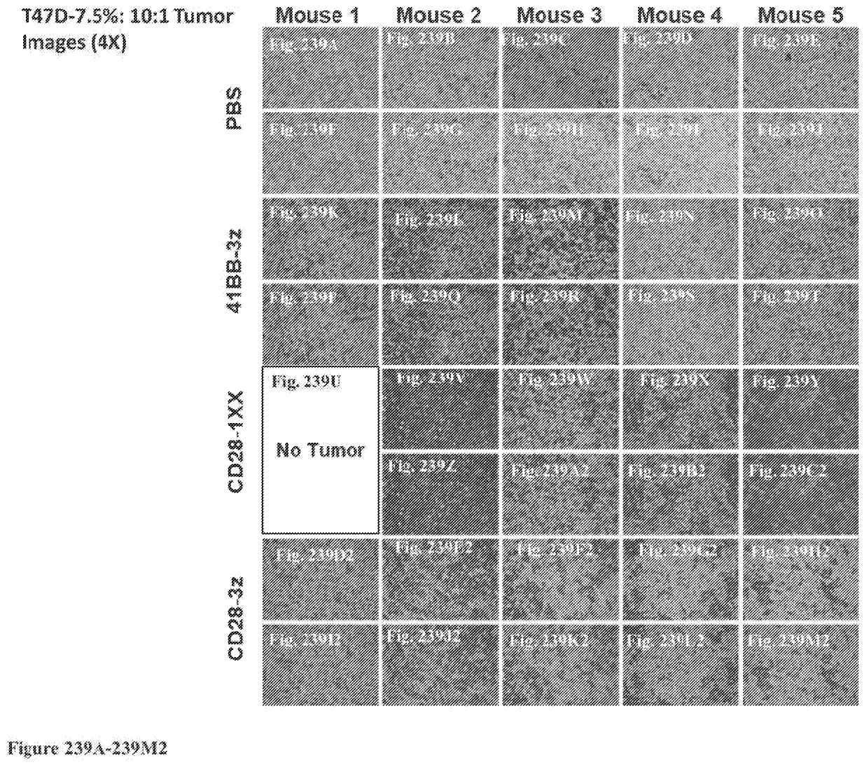

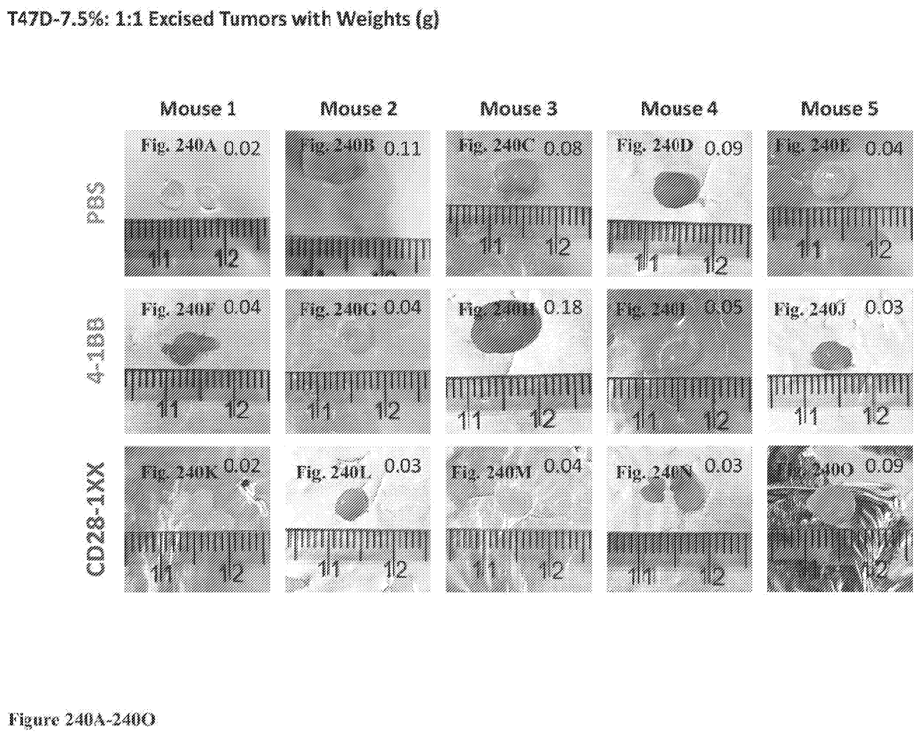

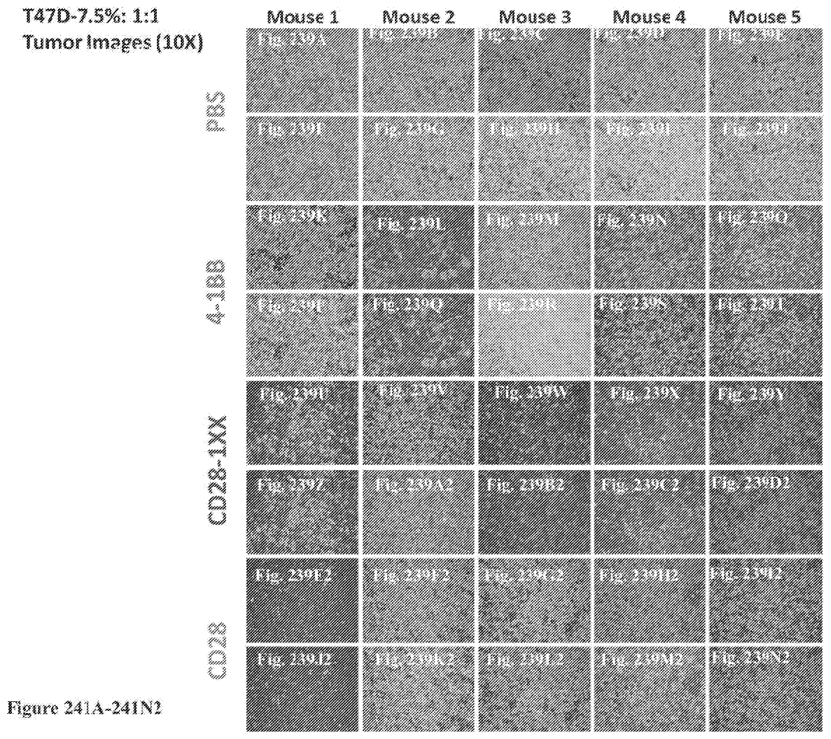

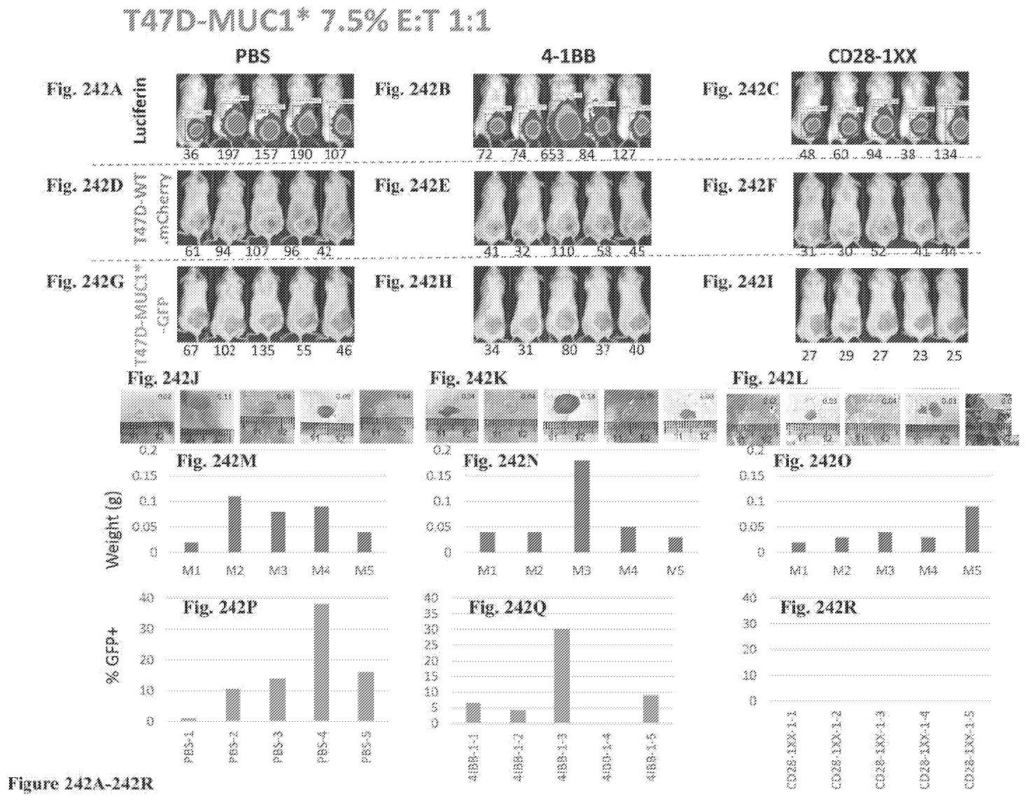

After sacrifice, the tumors were excised and analyzed as well. Excised tumors were photographed and weighed (FIG. 234, FIG. 236, FIG. 238, FIG. 240) weights were recorded. Tumors were then enzymatically dissociated and fluorescent photographs were taken to characterize and quantify the cells that caused the tumor recurrence. Recall that tumors were a heterogeneous population of high antigen expressing cells that were GFP positive and low antigen density cells that were mCherry positive. Red and green fluorescent photographs of the residual tumors show that when tumors have a high percentage of high antigen density cells and a high CAR T cell dose, the all the CAR T cells were able to kill the tumor cells that expressed high levels of the target antigen. However, tumor recurrence was mainly due to the growth of the low antigen density cells that standard CAR T cells do not kill as readily. When tumors were made up of a low percentage of high antigen cells and the CAR T cell dose was low, standard CAR T cells did no better than the control injection of PBS. However, the CAR T cells with the 1XX mutations in the CD3-zeta domain killed the low antigen density cells considerably better which greatly inhibited tumor recurrence (FIG. 235, FIG. 237, FIG. 239, FIG. 241).

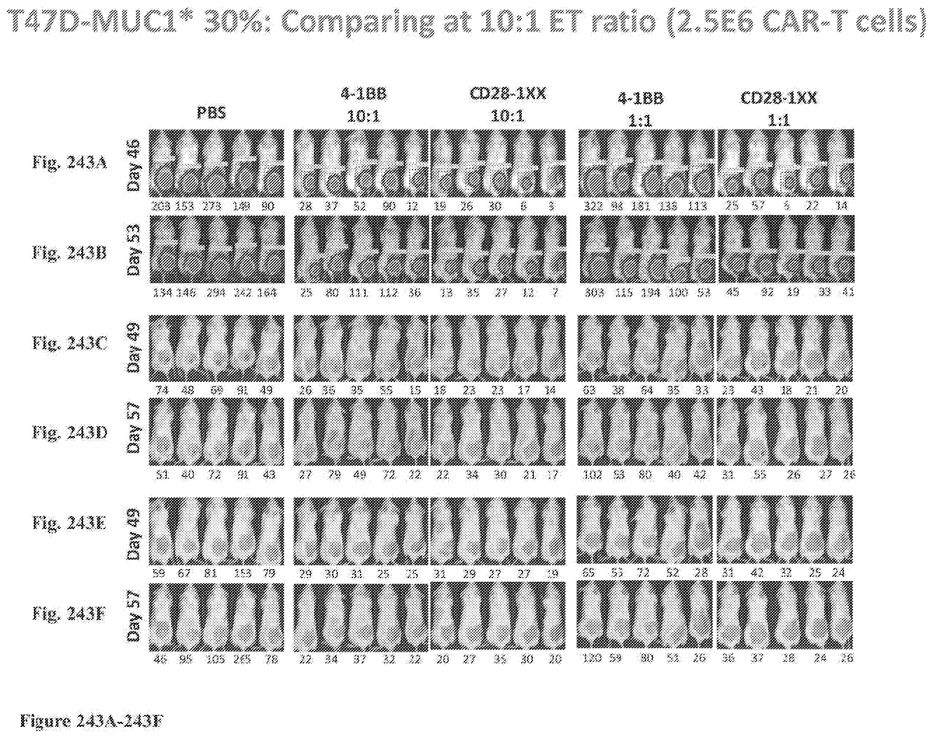

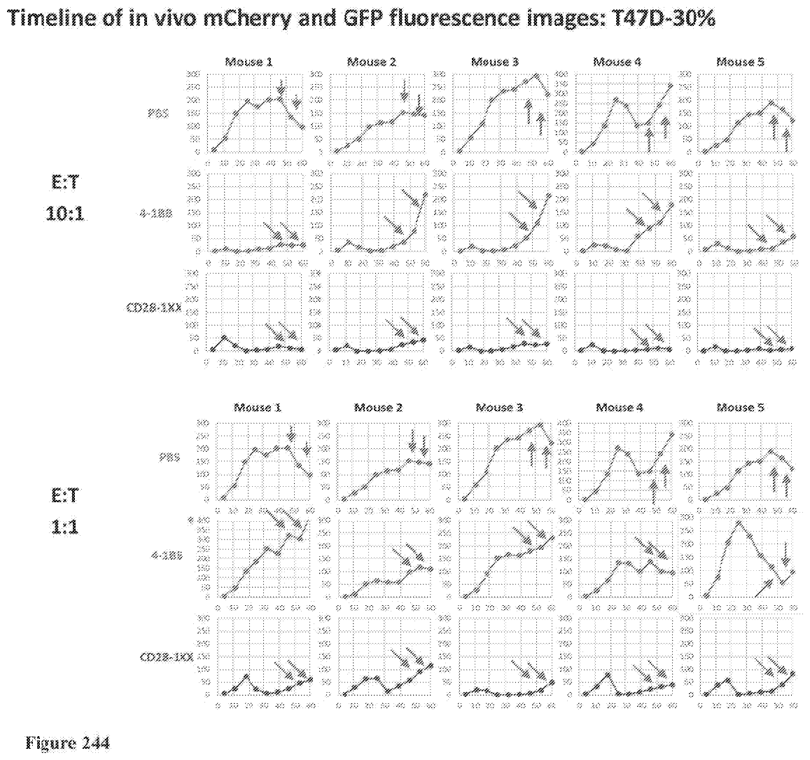

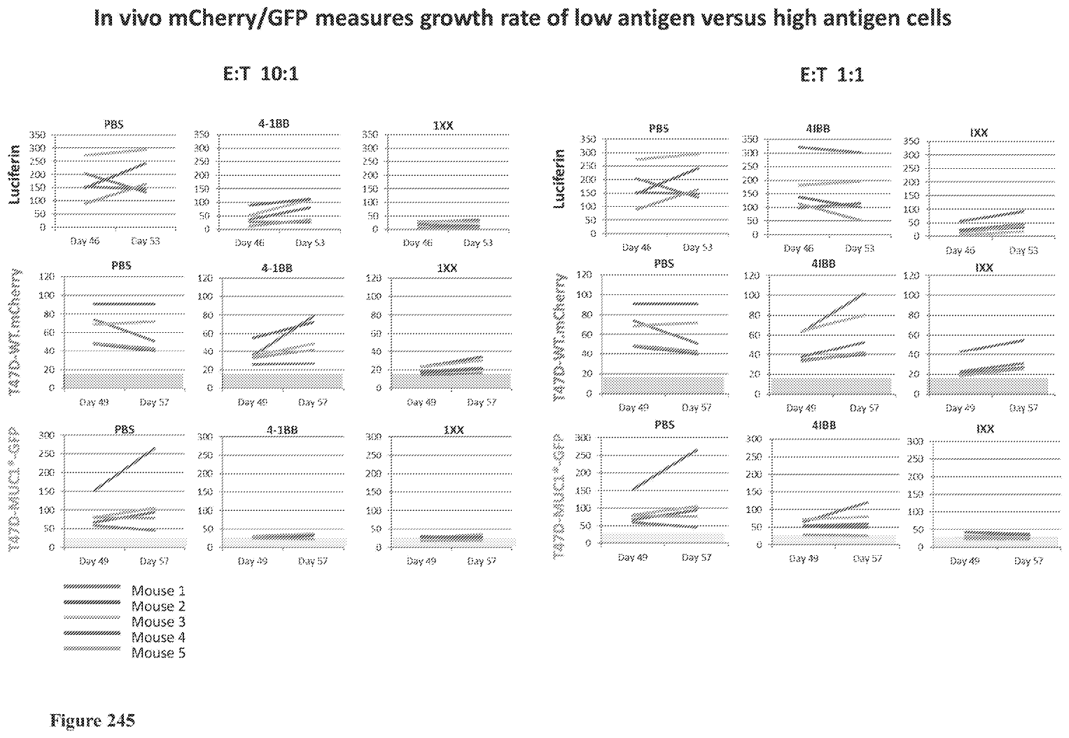

FIG. 242 shows photographs of live animals fluorescently imaged. Fluorescent photographs taken at two different timepoints showed that for animals implanted with a moderate percentage of high antigen cells and treated with a CAR with wild type CD3-zeta or a CAR with 1XX mutations, the growth rate of low antigen density cells was faster in the animals treated with the CAR with wild type CD3-zeta compared to animals treated with the CAR with the CD3-1XX. At low CAR T cell dose, the standard CARs showed a faster growth rate o both low and high antigen density tumor cells. This is consistent with the idea that a CAR with a wild type CD3-zeta is not as efficient as CARs with 1XX at killing low antigen density cells and that with a lower number of CAR T cells, the standard CARs get exhausted faster than CARs with the 1XX mutations in CD3-zeta (FIG. 242, FIG. 243, FIG. 244, FIG. 245.

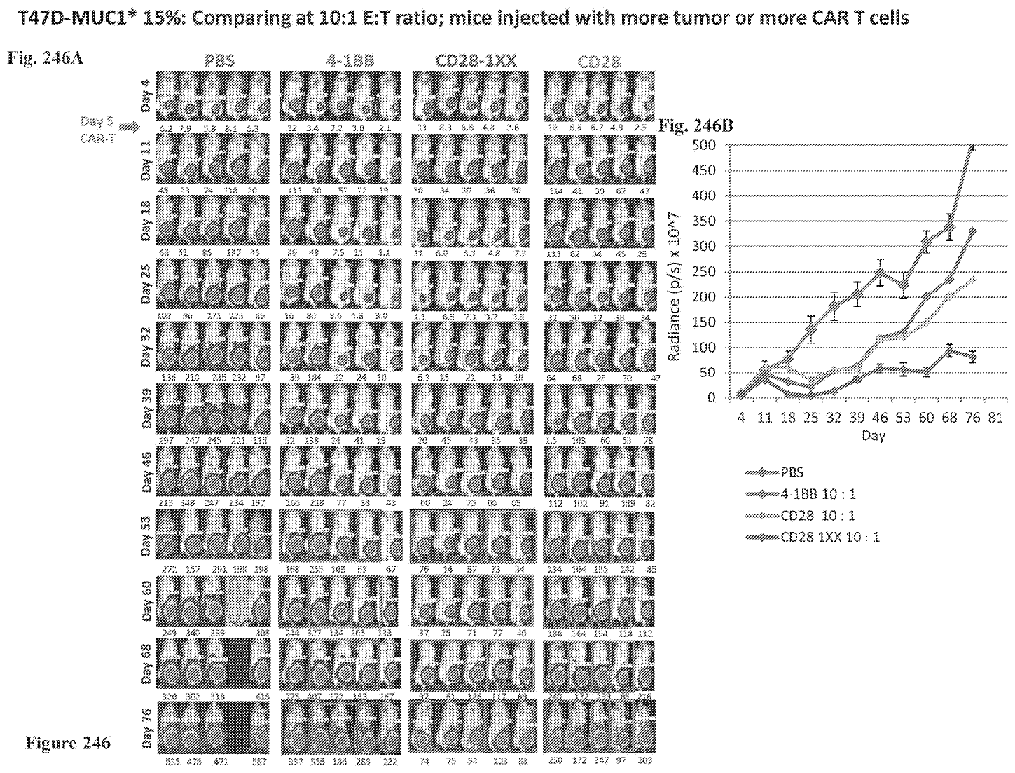

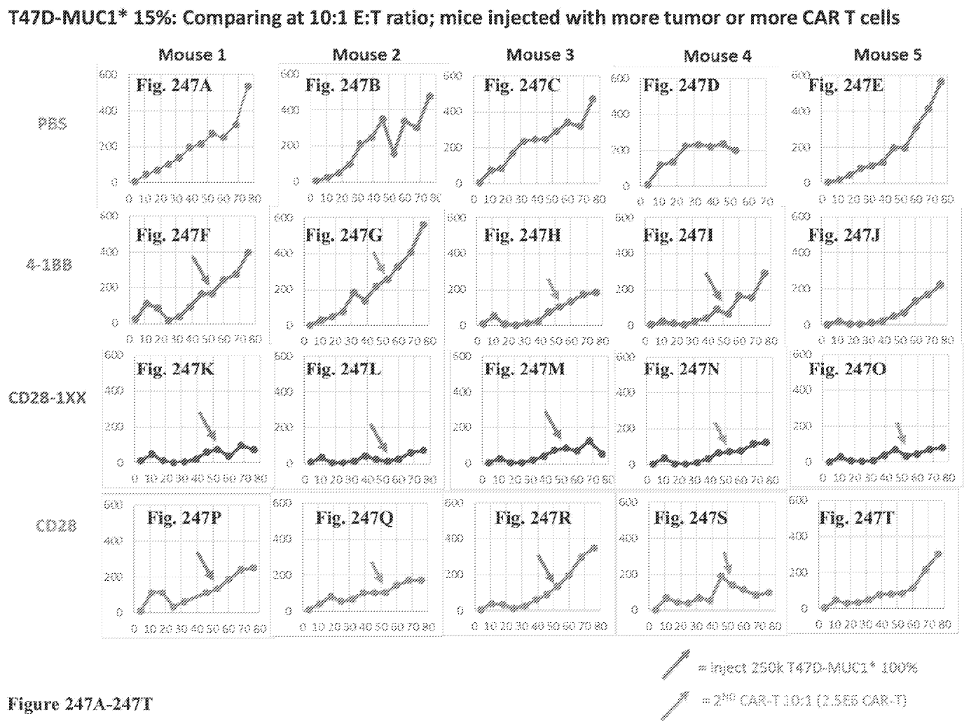

In another experiment, we injected mice at Day 52 with either 250,000 high antigen density tumor cells or 2,500,000 more CAR T cells. As can be seen in FIG. 246 and FIG. 247, the injection of additional tumor cells does not increase the tumor growth in animals treated with huMNC2-CD28-1XX CAR T cells. However, the injection of additional tumor cells does increase tumor growth in animals treated with huMNC2-CD28-3z or huMNC2-41BB-3z CAR T cells. This result is consistent with CAR T cells with 1XX mutations in CD3z prolong CAR T cell persistence in vivo. It can also be seen that the injection of additional CAR T cells suppressed tumor cells in some of the animals in all groups. This is consistent with the idea that tumor recurrence was not due to tumor escape because the fresh CAR T cells still recognized the tumor cells and killed them.

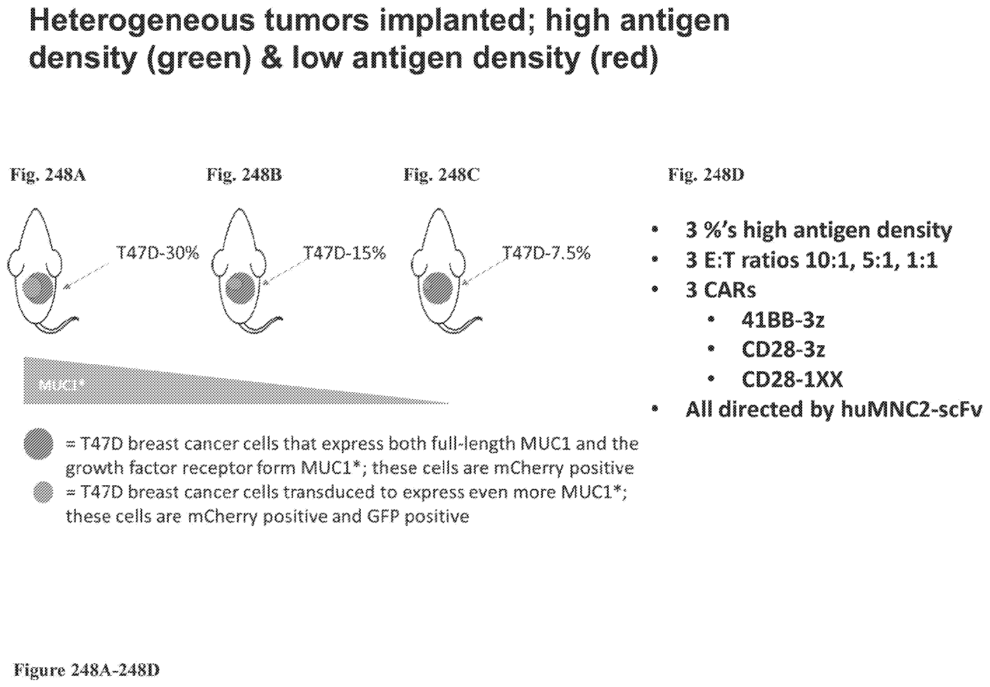

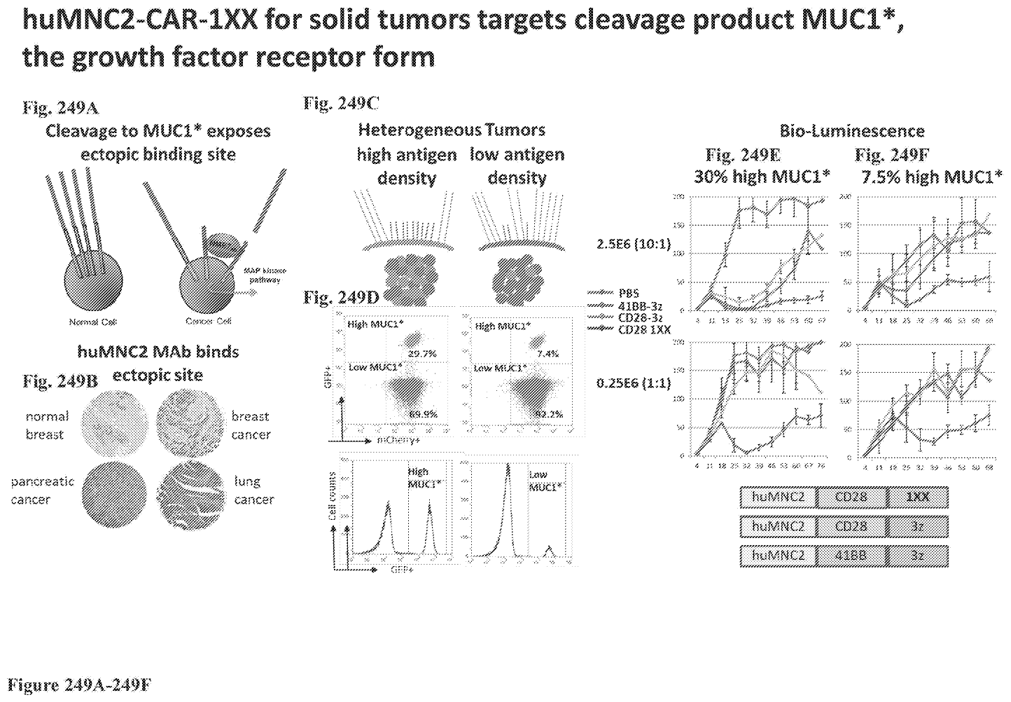

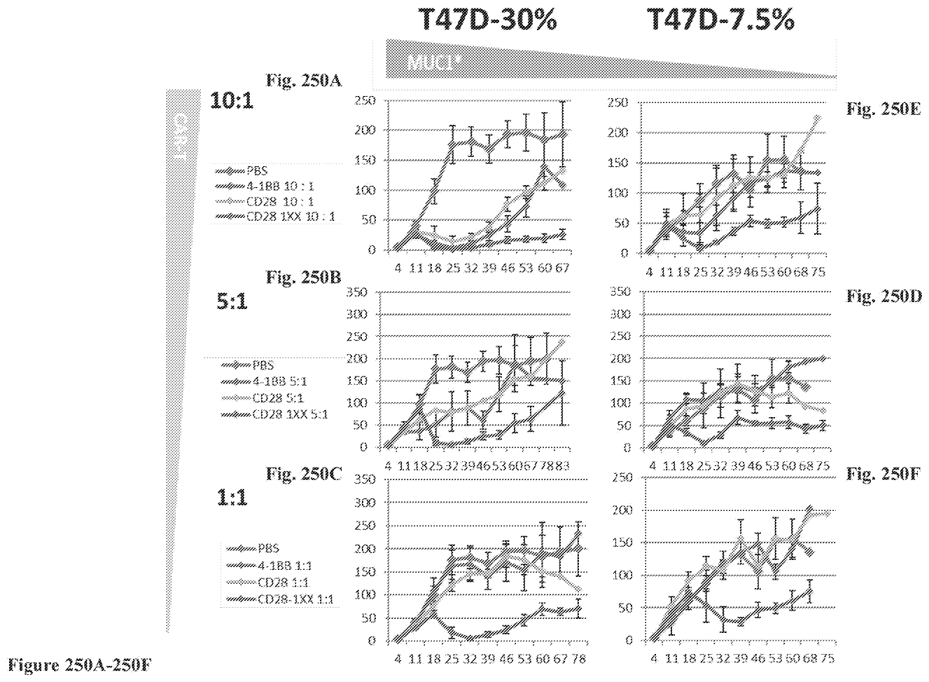

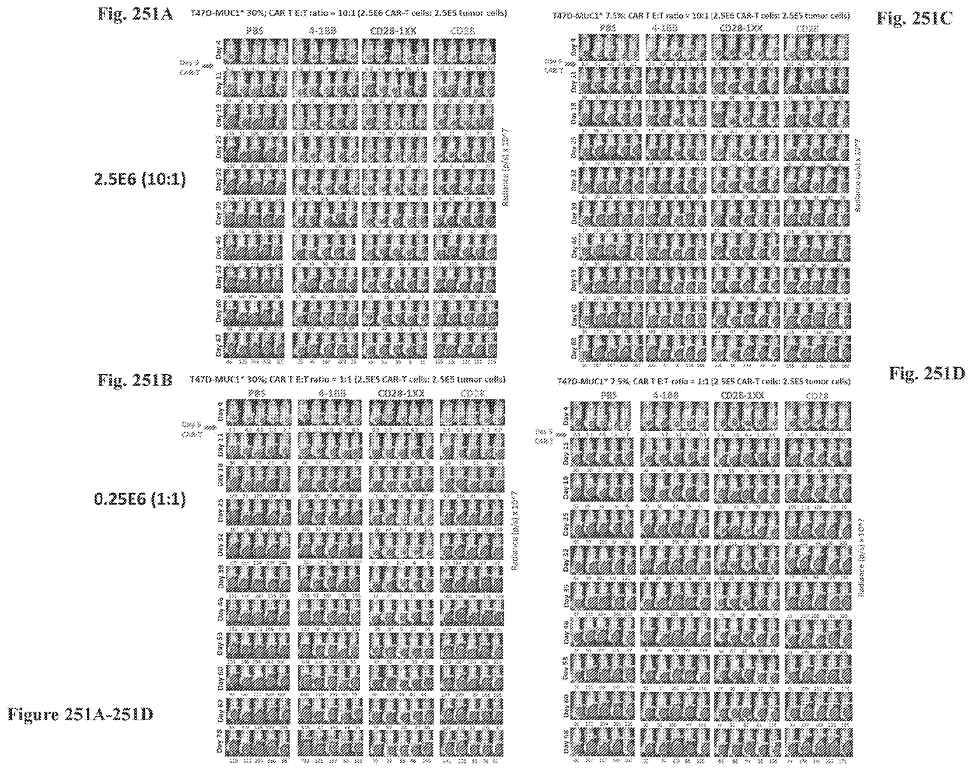

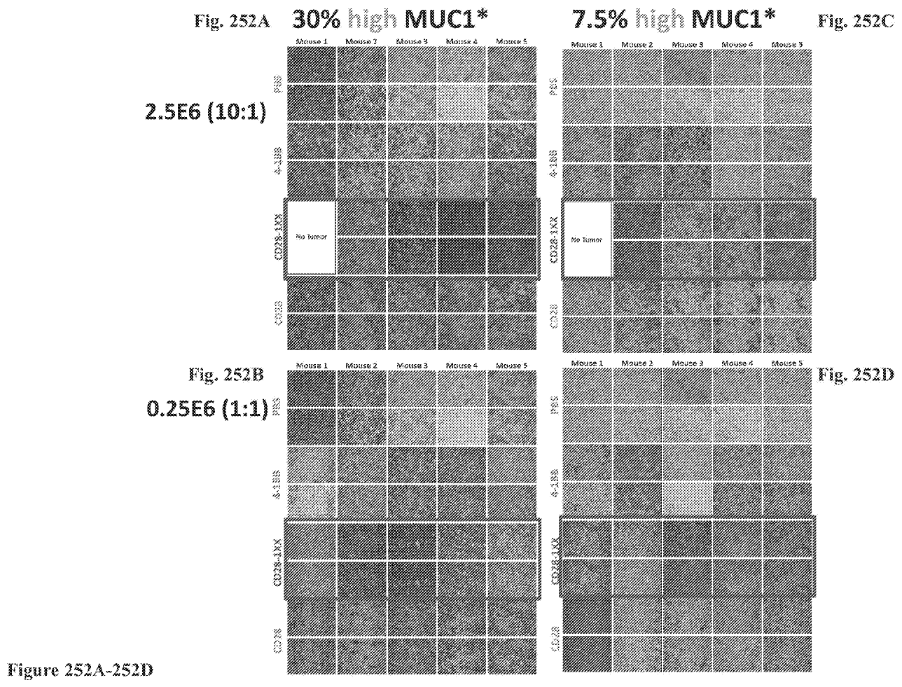

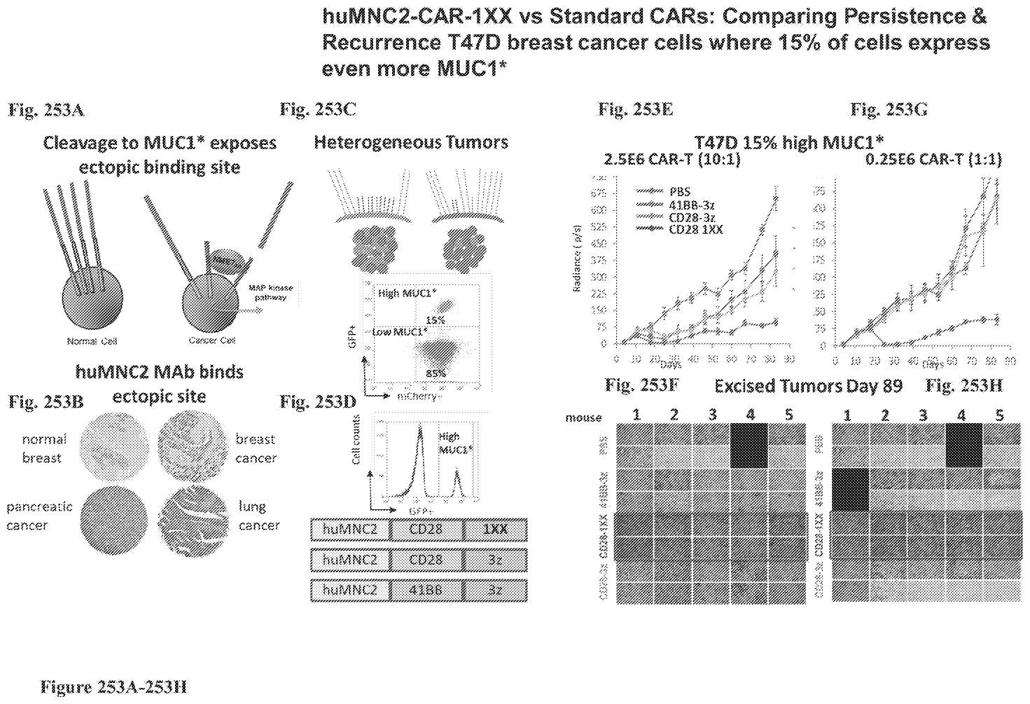

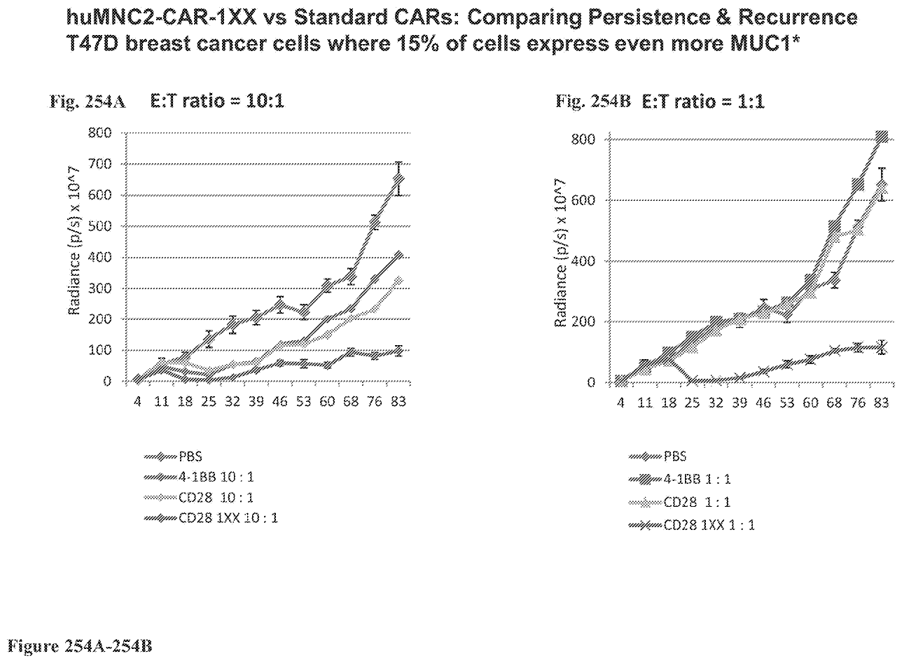

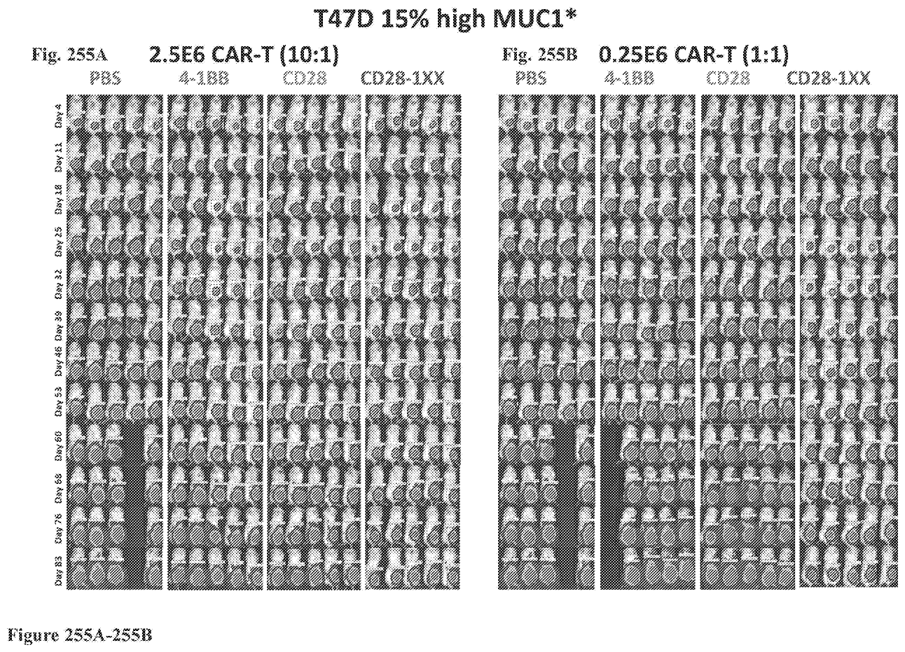

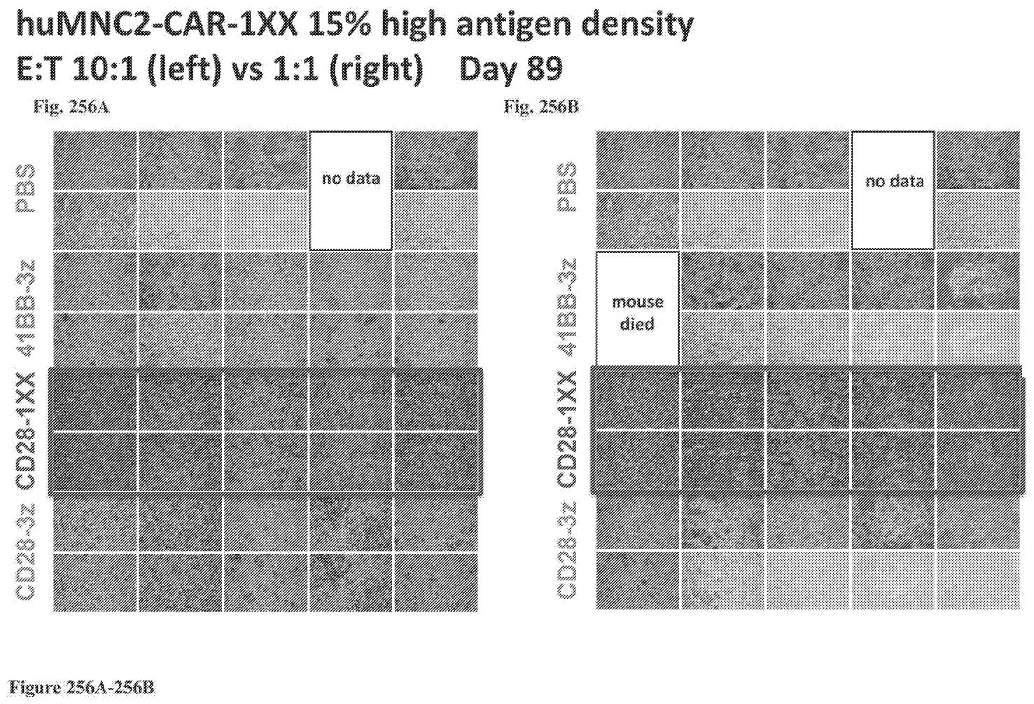

A significant problem for the treatment of cancers are the tumor cells that express low levels of tumor-associated antigen, especially with regard to cellular therapies where, to date, the killing of tumor cells has been dependent on the antigen density of the tumor cells. Tumor cells expressing low levels of the target antigen escape CAR T cells as well as engineered CAR NK cells. Essentially all solid tumors are heterogeneous and comprised of cells that express different levels of the target antigen. To further study this problem and to develop therapeutics that are able to detect and kill tumor cells expressing low levels of the target antigen, we devised a strategy for making heterogeneous tumors made up of both high and low antigen expressing cells, wherein the two different cell types bear fluorescent labels that can be detected in vivo as well as ex-vivo after sacrifice of the test animals (See FIG. 248-FIG. 256). As can be seen in the IVIS graphs of FIG. 249 and FIG. 250, CARs with standard CD3z signaling domains or with 1XX mutations kill tumor cells expressing high levels of antigen when treated with high, 10:1, dose of CAR T cells. However, for CARs with standard CD3-z domains, the difference between treated and untreated wanes for animals implanted with tumor cells expressing low percentage of high antigen expressing cells, even when the dose of CAR T cells is high. This result argues that CARs with mutated CD3-z signaling domain, like 1XX, have enhanced ability to detect tumor cells that express low percentages of high antigen expressing cells. FIG. 251 shows that in animals that were implanted with 250,000 tumor cells, then treated once with either 2.5M CAR T cells or 250,000 CAR T cells, tumors recur and the timing and degree of recurrence is greater in animals treated with CARs bearing standard CD3z than in animals bearing CD3z with 1XX mutations. The excised tumors were fluorescently photographed at mCherry wavelength and at GFP wavelength. As can be seen in FIG. 252 tumor recurrence is minimal for the tumors of animals treated with huMNC2-CD28-1XX. Further, the figure shows that tumor recurrence is dominated by the low antigen expressing cells that are missed by the standard CAR T cells. As can be seen in FIG. 253 and FIG. 256, when tumors expressing low percentage of high antigen expressing cells are coupled with treating with low dose CAR T cells, a combination of CAR T cell exhaustion plus escape of low antigen cells drives tumor resistance. We note that for the tumors labeled 15% high antigen density, flow cytometry showed that a population of tumor cell clusters was present, which has been reported to result in a greater rate of tumor growth in vivo.

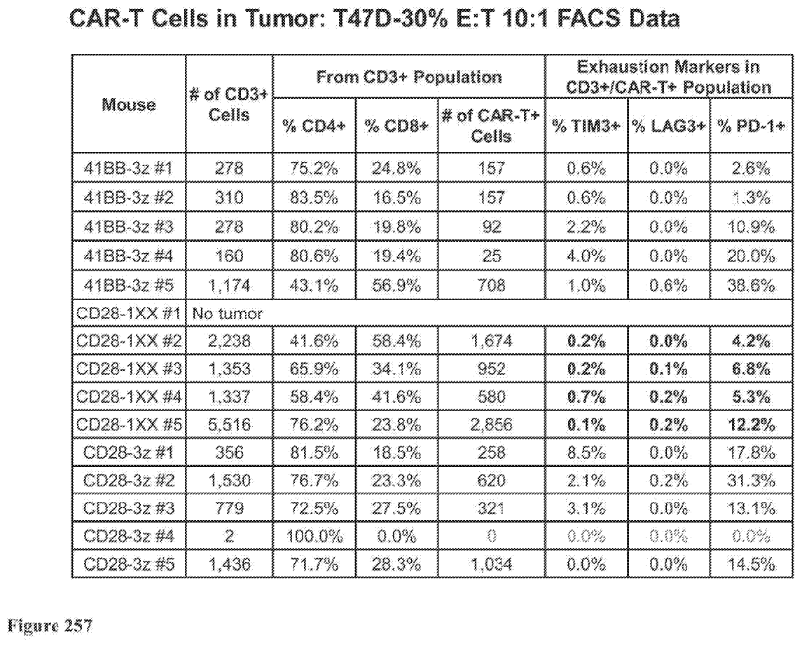

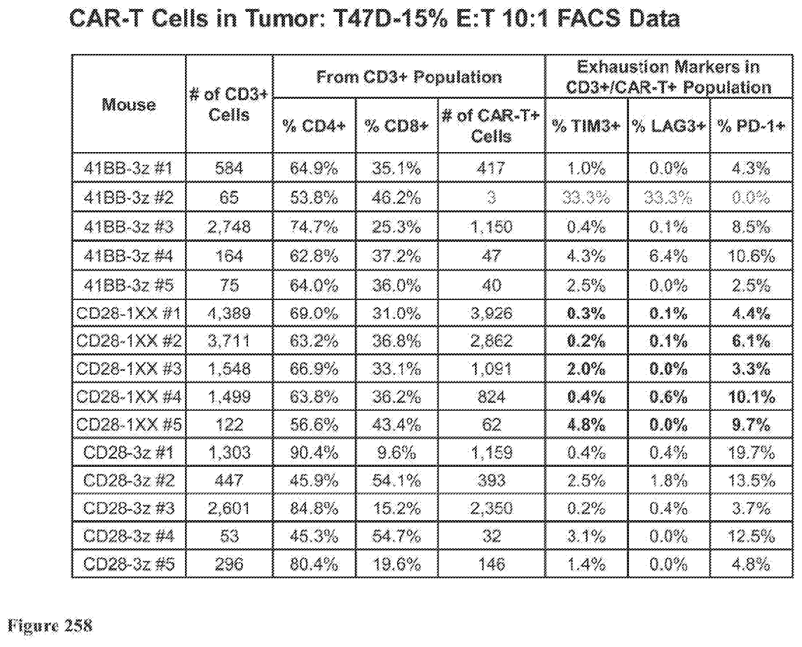

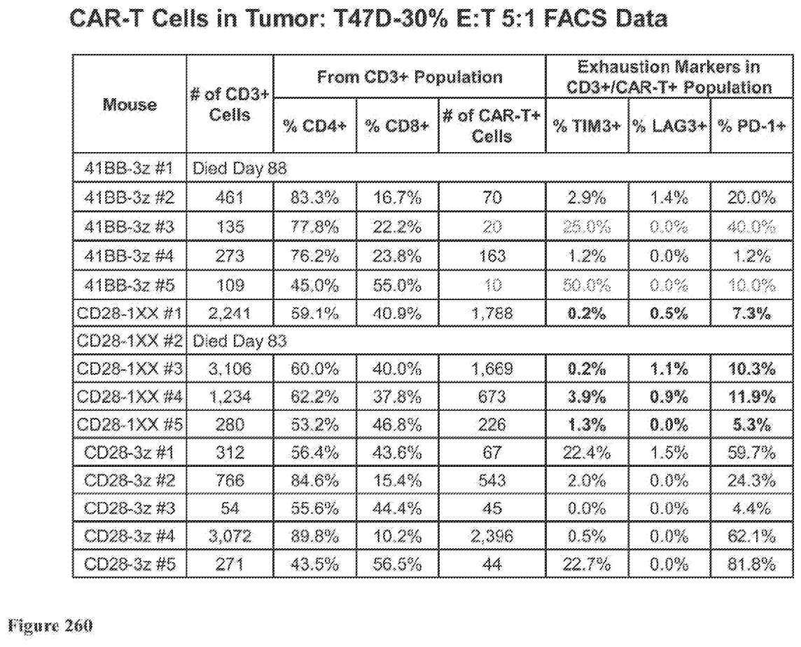

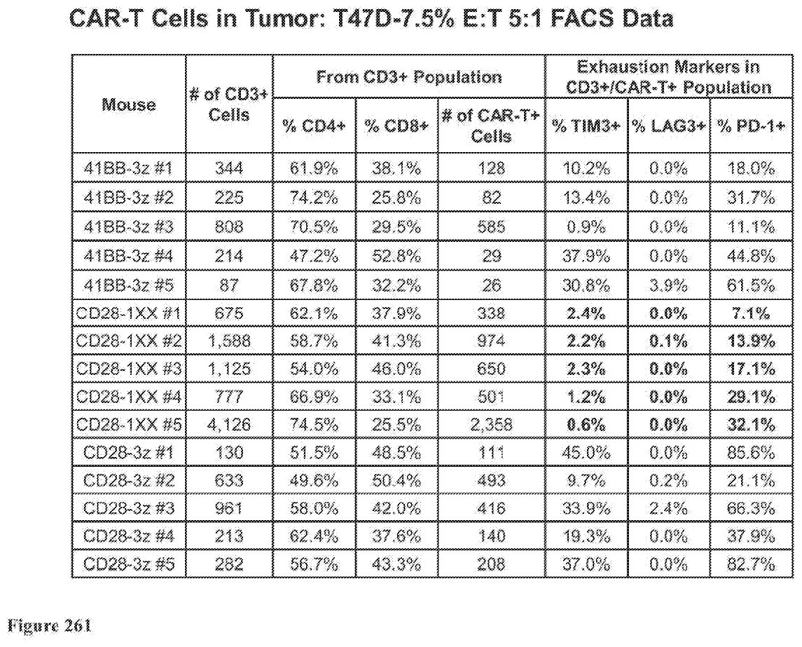

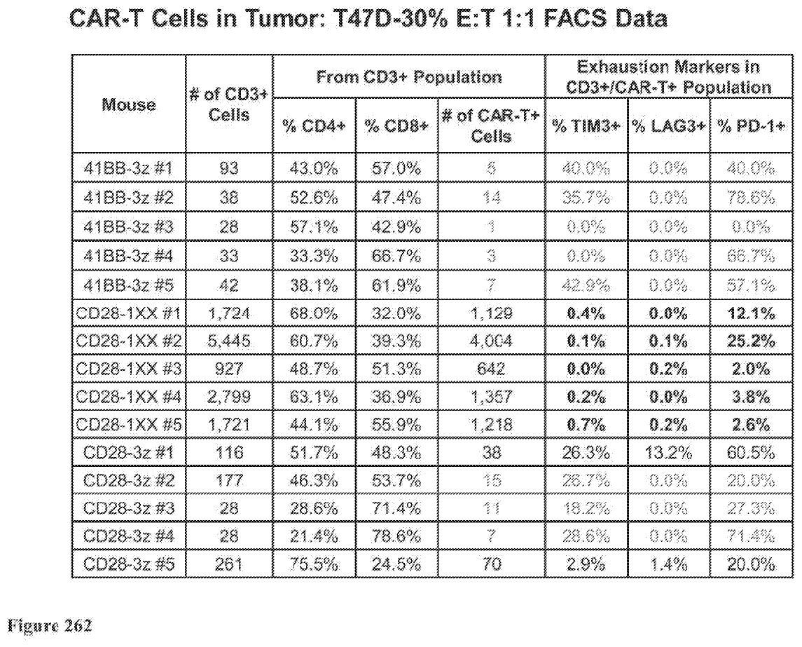

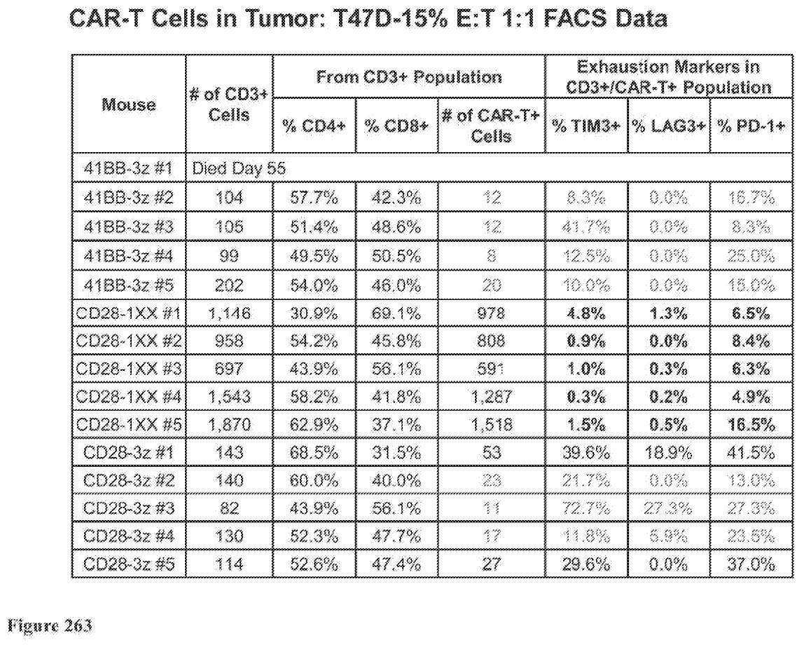

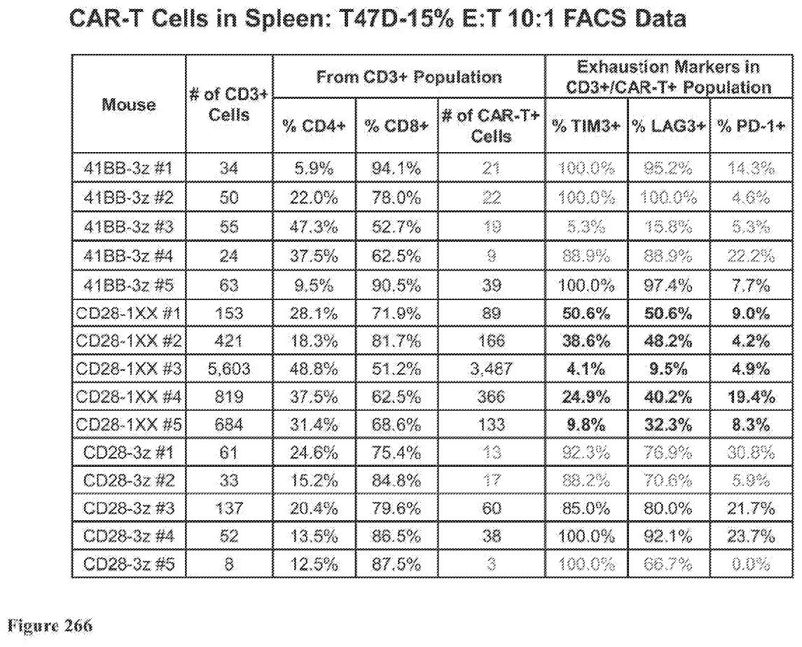

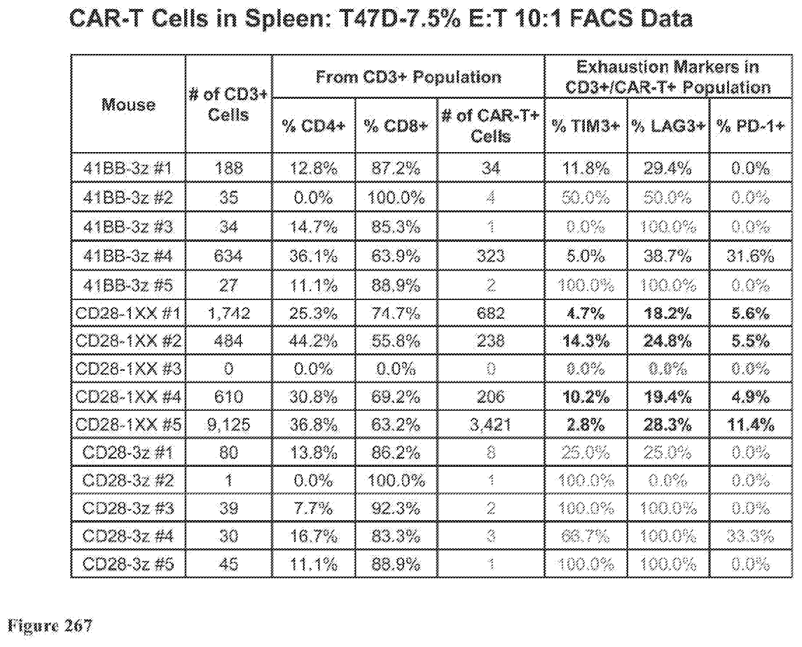

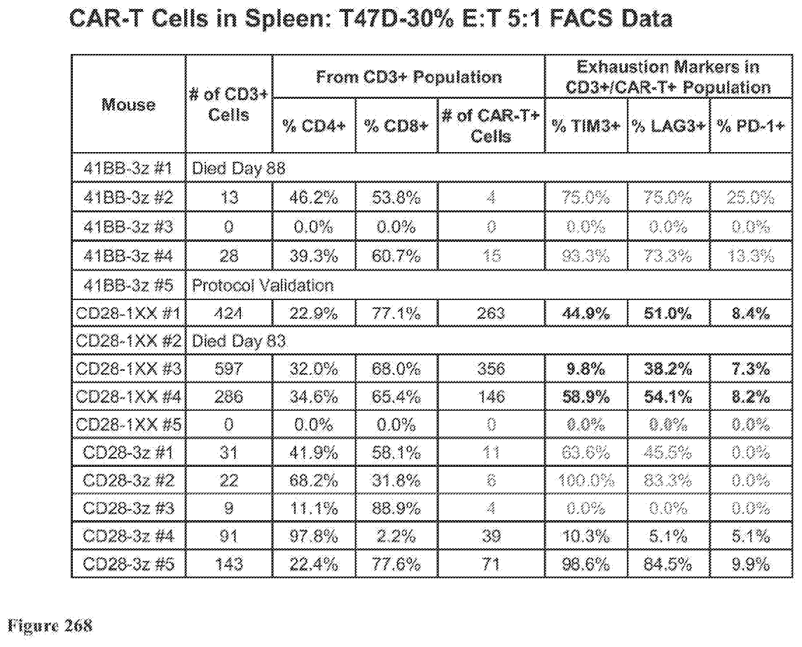

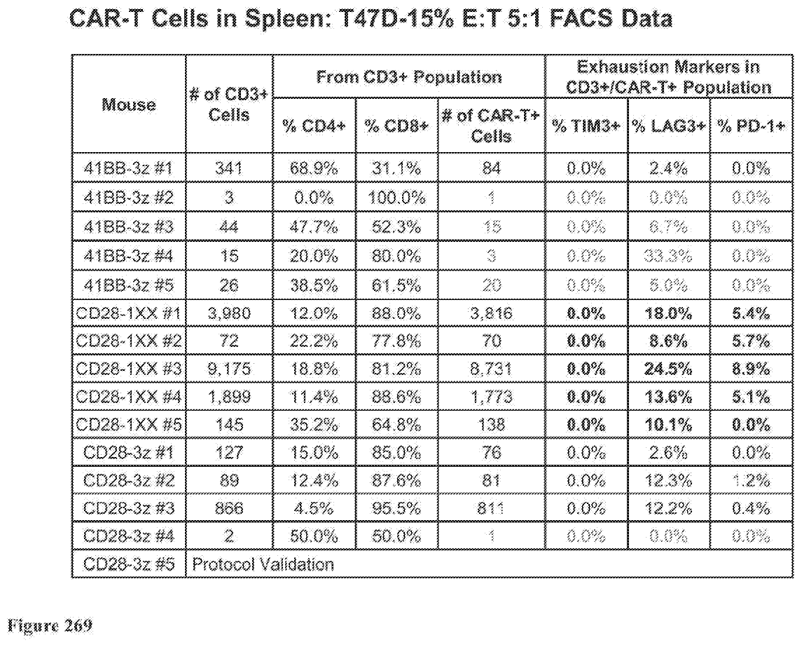

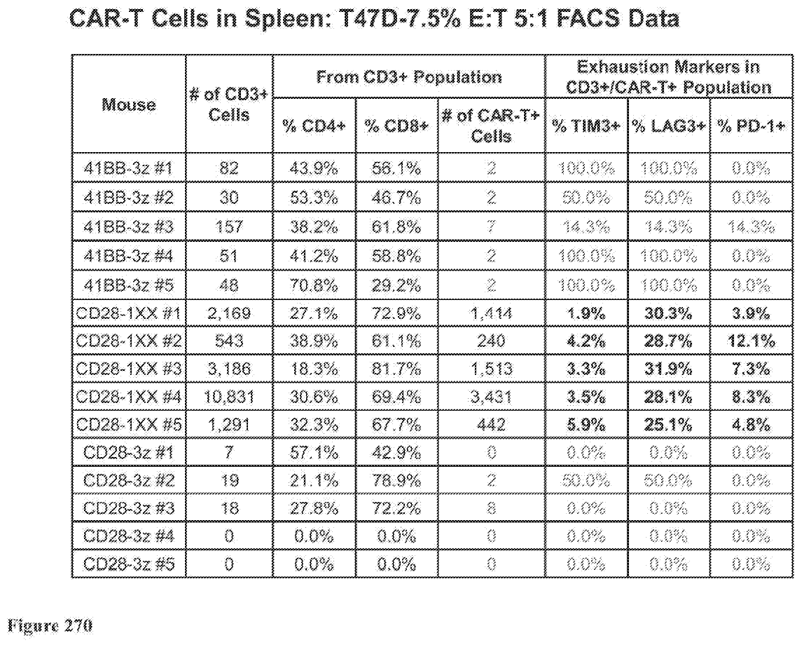

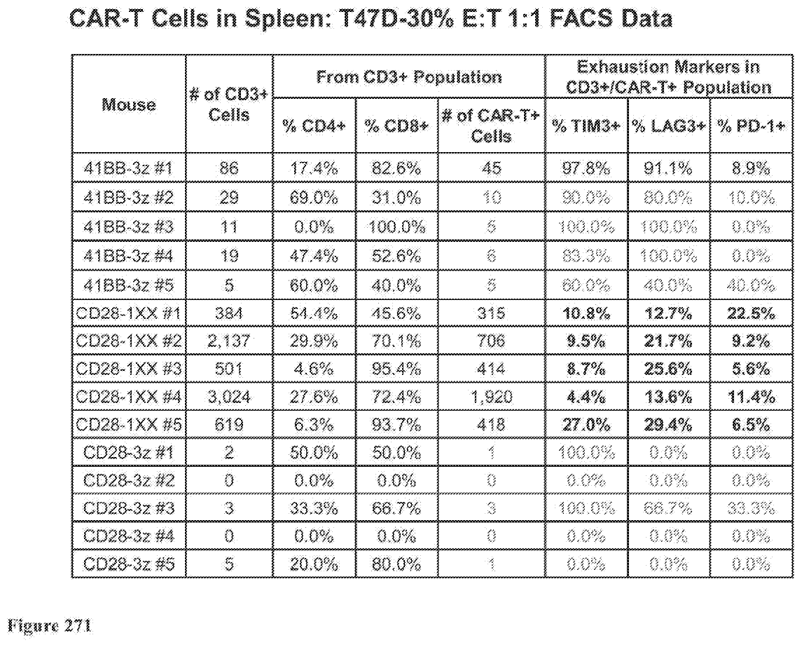

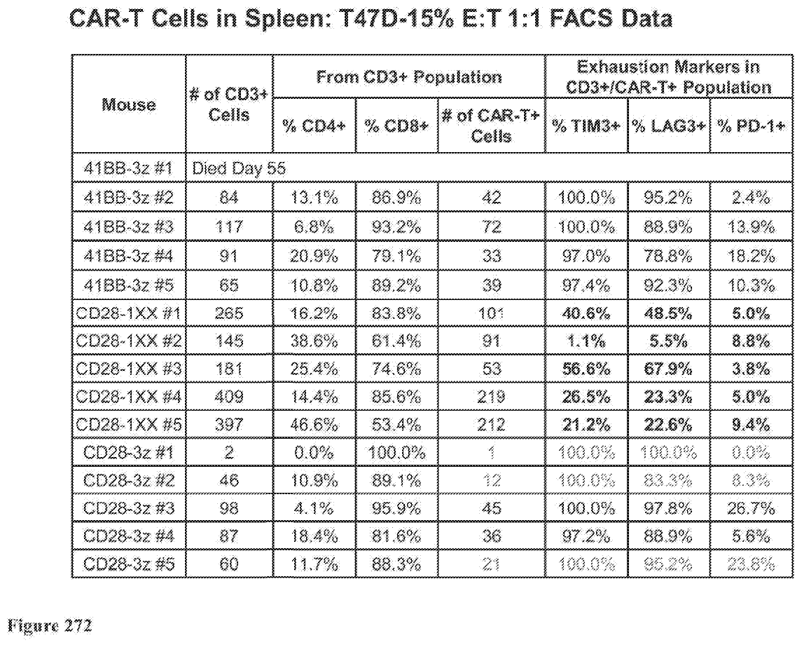

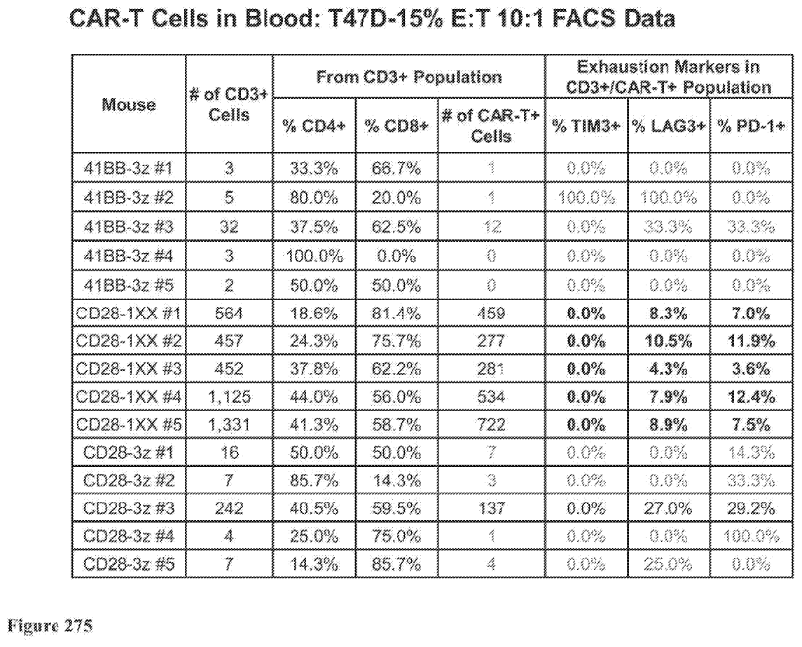

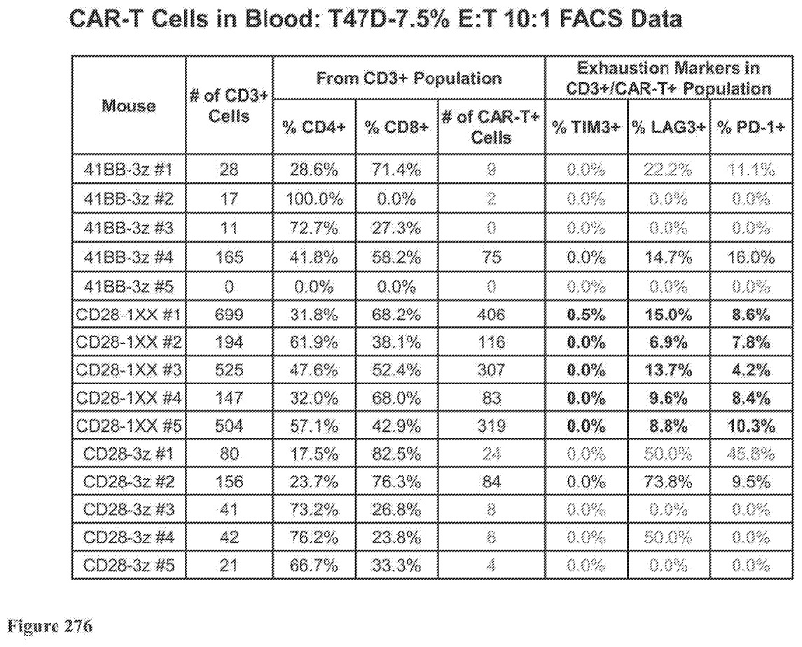

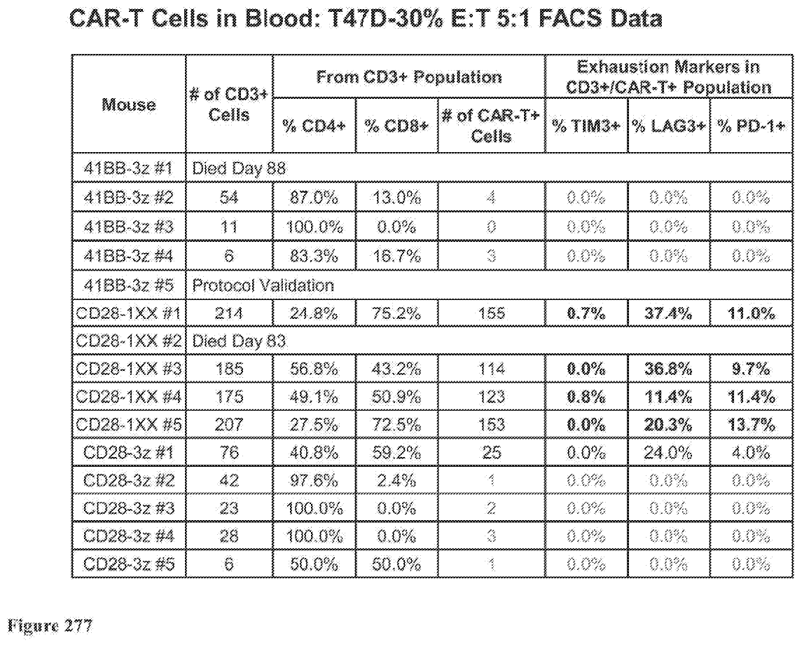

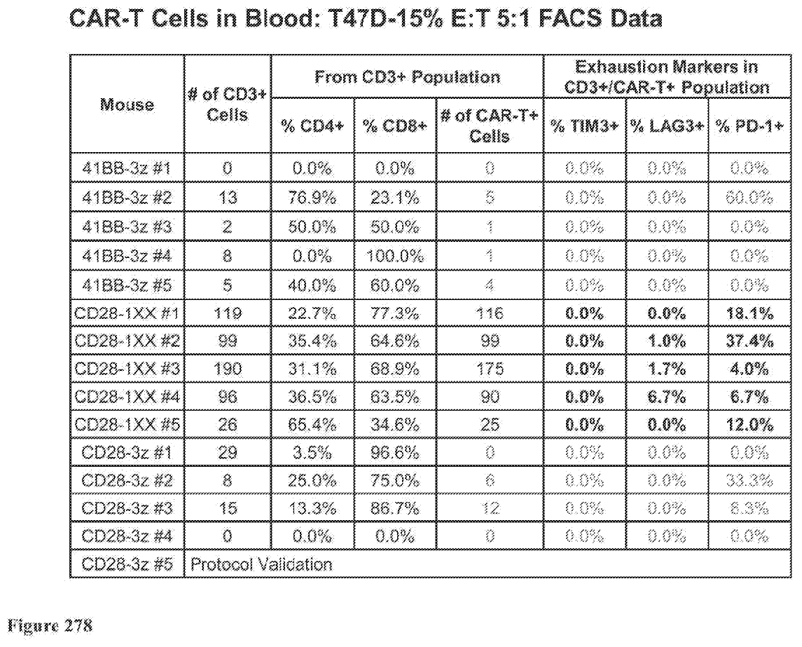

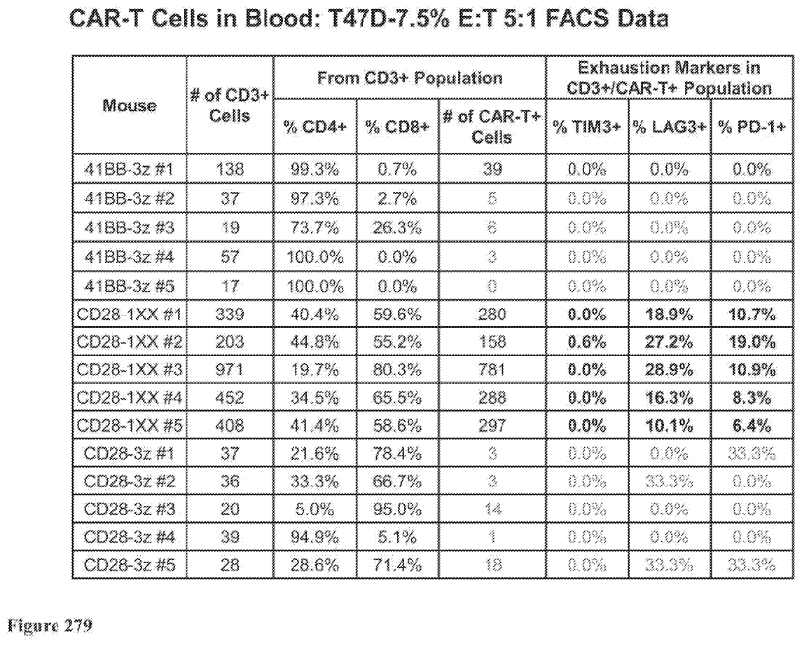

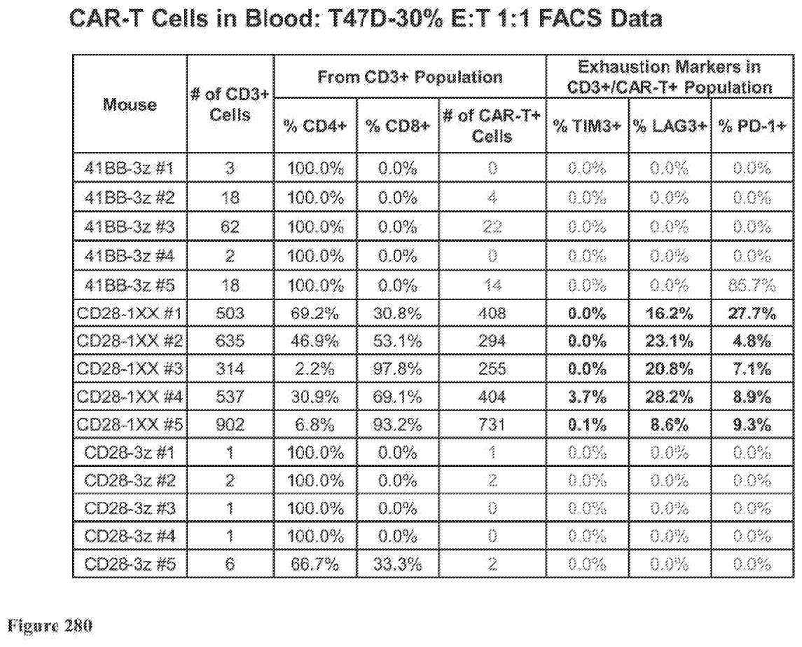

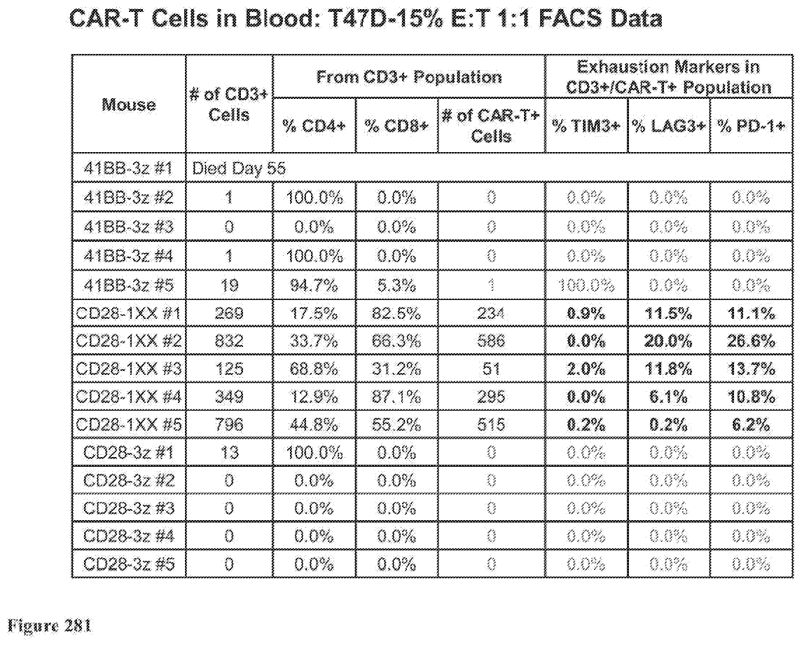

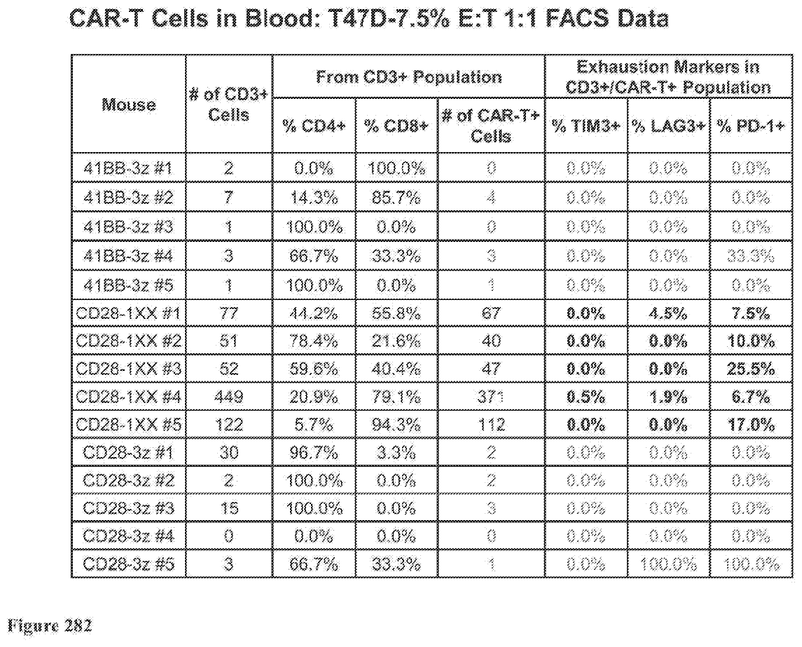

Anti-MUC1* CAR T cells persist longer and stave off T cell exhaustion when the CD3-zeta signaling domain is mutated to slow signaling as in mutating some of the Tyrosines, for example as we have done with the huMNC2-CD28-1XX, see Tables in FIG. 257-FIG. 282. Excised tumors from the test animals were analyzed by flow cytometry for the presence of human CAR T cells and their expression of exhaustion markers. It is notable that even in a high antigen density tumor, such as 30% of the cells express high levels of the antigen, in this case MUC1*, wherein animals were treated with a high effector to target ratio of 10:1 CAR T cells to tumor cells, there is a significantly higher number of CAR T cells in the tumors of mice treated with the anti-MUC1* CAR T with CD3z mutations, in this case 1XX. As can be seen in the Table of FIG. 257, the mice treated with huMNC2-CD28-1XX had an average of 1,516 CAR T cells in the excised tumor, compared to only 196 CAR T cells for the huMNC2-41BB-3z and 395 CAR T cells for the huMNC2-CD28-3z treated mice. In the huMNC2-CD28-1XX treated mice, the CAR T cells expressed the lowest percentages of exhaustion markers. That means that for tumors with a significant amount of high antigen expressing cells and at high dose of CAR T cells, the mutated CD3z, as in 1XX mutations, gives the CAR T cells an advantage in terms of being able to recognize tumor cells and also being able to stave off exhaustion. Comparison of CAR T cell persistence among CARs, with or without mutated CD3z, is assessed by looking at these same 30% high antigen expressing tumors, but wherein animals were treated with a low dose of CAR T cells: 250,000 CAR T cells and 250,000 tumor cells, i.e. 1:1 ratio. The Table of FIG. 262 shows that CARs with a standard CD3z signaling domain, administered to animals at low dose have almost no measurable CAR T cells in their tumors after about 70-90 days after CAR T cell injection. These data argue that at low CAR T dose, the CAR T cells need to work harder and get exhausted faster. In contrast, animals treated with low dose of 250,000 huMNC2-CD28-1XX CAR T cells to 250,000 implanted tumor cells had on average 1,444 CAR T cells in their tumors at day of sacrifice, which is about the same as when the animals were treated with high dose of huMNC2-CD28-1XX CAR T cells. This result shows that the mutated CD3z signaling domain resists T cell exhaustion.

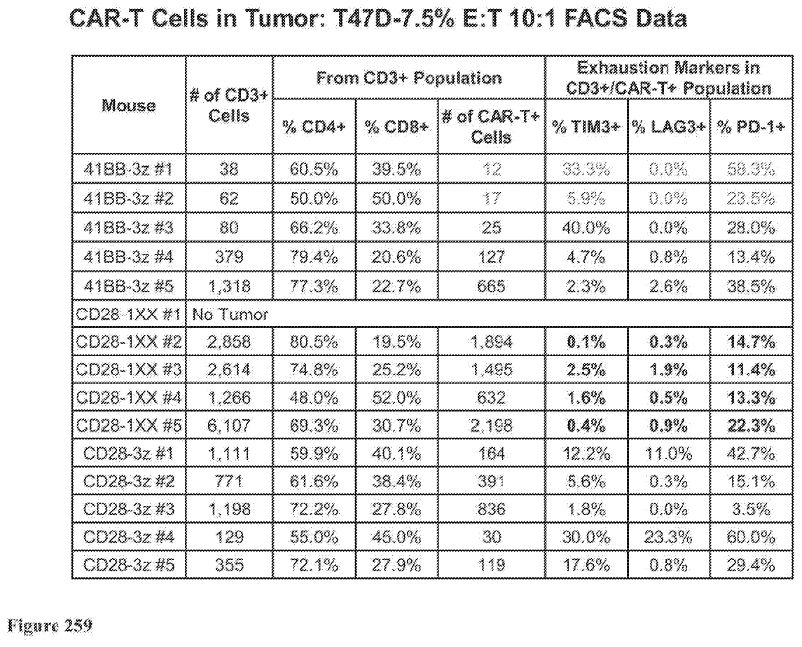

However, looking at the excised tumors from animals implanted with tumors that only have 7.5% expressing high levels of antigen but treated with high dose of CAR T cells-2.5M CAR T cells to 250,000 implanted tumor cells—shows that mutations that slow the signaling of CD3-z also do something else. It follows that with low antigen density but high dose of CAR T cells, the CAR T cells would signal less due to lesser numbers of antigen molecules and thus would not get exhausted. Unexpectedly, as can be seen in the Table of FIG. 259, 7.5% high antigen expressing tumors excised from animals treated with high, 10:1 dose of CAR T cells still do not have significant numbers of CAR T cells in their tumors if treated with CARs having wild type CD3z. Tumors from animals treated with huMNC2-CD28-1XX had on average 1,555 CAR T cells in the excised tumor, compared with only 167 in tumors from huMNC2-41BB-3z treated mice and 275 in tumors from huMNC2-CD28-3z treated mice. This result strongly argues that, unexpectedly, CAR T cells with a 1XX mutated CD3z, or similar mutated signaling domain, have enhanced ability to recognize and kill tumor cells that express low levels of the target antigen.

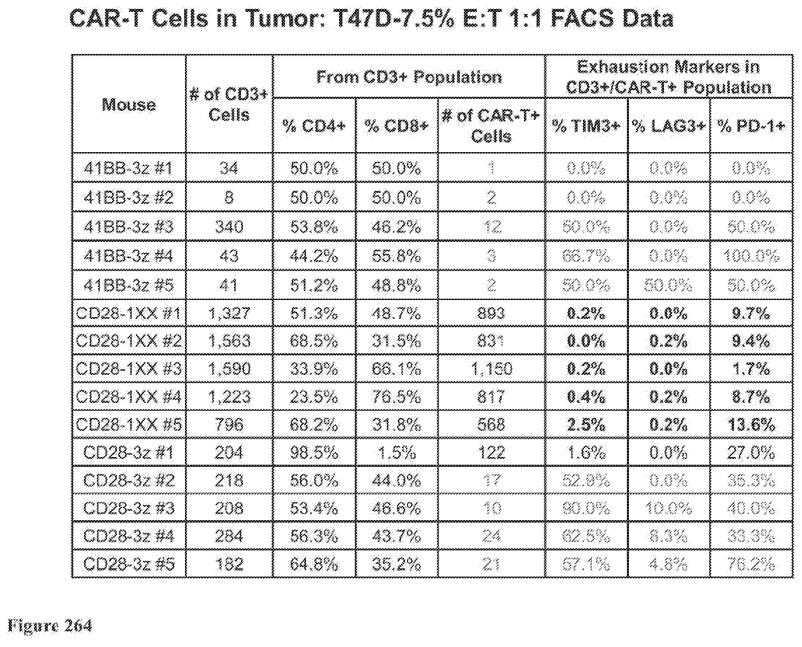

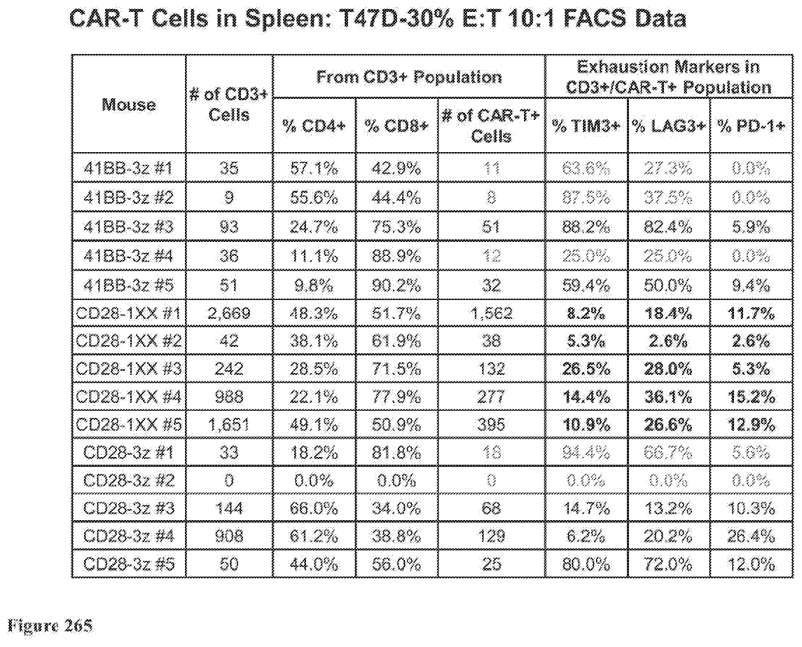

Similarly, standard CAR T cells could not recognize nor kill tumors that expressed a low percentage, 7.5%, of high antigen expressing cells when animals were also treated with a low dose of CAR T cells. The Table of FIG. 264 shows that of the mice implanted with tumors that were only 7.5% high antigen expressing cells that were treated with low dose CAR T cells (250,000 CAR T cells and 250,000 tumor cells, 1:1 ratio) only the animals treated with huMNC2-CD28-1XX had measurable CAR T cells in their tumors, 841 on average, and those CAR T cells showed almost no signs of exhaustion.

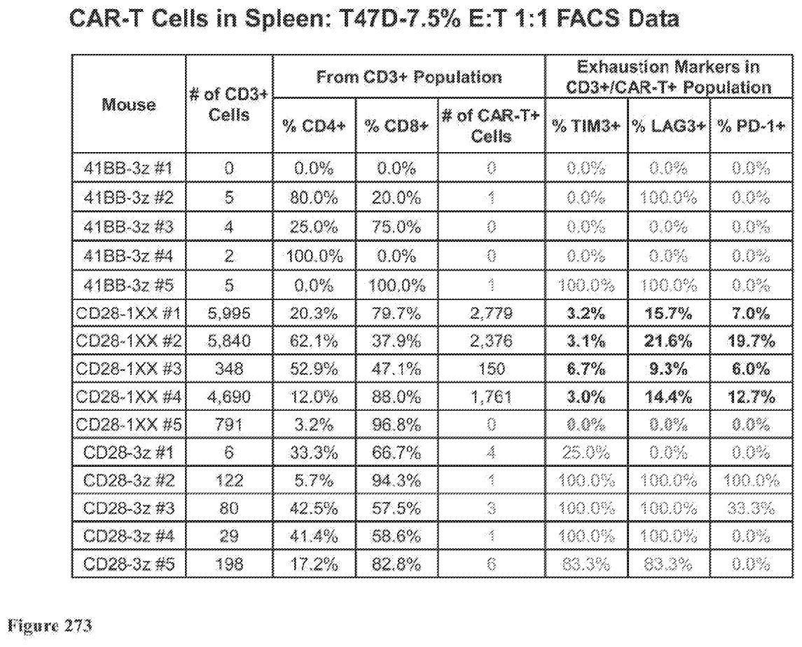

Similar results were obtained when cells from the spleens of the animals were analyzed (FIG. 265-FIG. 273). One striking example is shown in FIG. 273. Analysis of spleens excised from animals bearing tumors that were 7.5% high antigen expressing cells when implanted, showed that only spleens from animals treated with huMNC2-CD28-1XX had detectable CAR T cells, wherein they had on average 1,413 CAR T cells in their tumors.

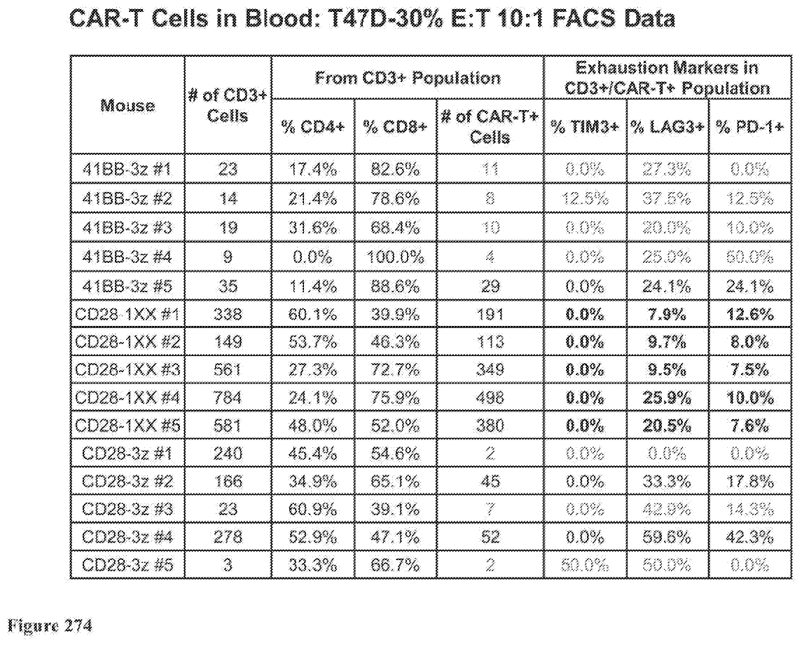

The trend was also observed when the blood from the treated animals was analyzed by flow cytometry as shown in FIG. 274-FIG. 282.

These experiments demonstrate that anti-MUC1* CAR T cells kill MUC1* positive tumor cells and that the killing is enhanced when the CAR incorporates a mutated CD3z domain such as one with the 1XX mutations. The experiments also show an unexpected result that CAR T cells with the 1XX CD3z domain have enhanced ability to recognize and kill tumor cells that express low levels of the target antigen. Thus, CAR T cells bearing 1XX, including but not limited to CARs that recognize MUC1*, have improved killing of solid tumor cancers, where tumors are more heterogeneous than blood cancers. Also, CAR T cells bearing 1XX, including but not limited to CARs that recognize MUC1*, are far better than standard CAR T cells at preventing tumor recurrence.

One aspect of the invention is a method for treating a patient diagnosed with, suspected of having, or at risk of developing a MUC1 positive or MUC1* positive cancer, wherein the patient is administered an effective amount of immune cells that have been transduced with a MUC1* targeting CAR. In another aspect of the invention, the immune cells are T cells isolated from a patient, which are then transduced with CARs wherein the targeting head of the CAR binds to MUC1*, and after expansion of transduced T cells, the CAR T cells are administered in an effective amount to the patient. In yet another aspect of the invention, the immune cells are T cells isolated from a patient, which are then transduced with CARs wherein the targeting head of the CAR comprises portions of MNC2, MNE6, 20A10, 3C2B1, 5C6F3, 25E6, 18G12, 28F9, 1E4, B12, B2, B7, B9, 8C7F3, or H11, and after optional expansion of transduced T cells, the CAR T cells are administered in an effective amount to the patient. In another aspect of the invention, the antibody fragment of MNC2, MNE6, 20A10, 3C2B1, 5C6F3, or 25E6 is incorporated into a chimeric antigen receptor of a natural killer cell, or NK cell.

### Specificity of Anti-MUC1* Targeting Antibodies

As these experiments demonstrate, the critical portion of a CAR is the antibody fragment that directs the immune cell to the tumor cell. As we will show in the following section, MNE6, MNC2, 20A10, 3C2B1 and 5C6F3 are highly specific for the form of MUC1* that is expressed on tumor cells. The next most important part of a CAR is the cytoplasmic tail bearing immune system co-stimulatory domains and the signaling domain CD3-zeta or variations thereof. The identity of these domains modulates the degree of immune response but does not affect the specificity. As shown, the identity of the transmembrane portion of a CAR is the least important. It appears that as long as the transmembrane portion has some flexibility and is long enough to allow the antibody fragment to reach its cognate receptor on the tumor cell, it will suffice. CARs comprising the MNE6 targeting antibody fragment, and intracellular co-stimulatory domains 41BB and CD3-zeta but having a variety of different extracellular, transmembrane and short cytoplasmic tail all worked in that they specifically killed the targeted cells while stimulating the expansion of the host T cells.

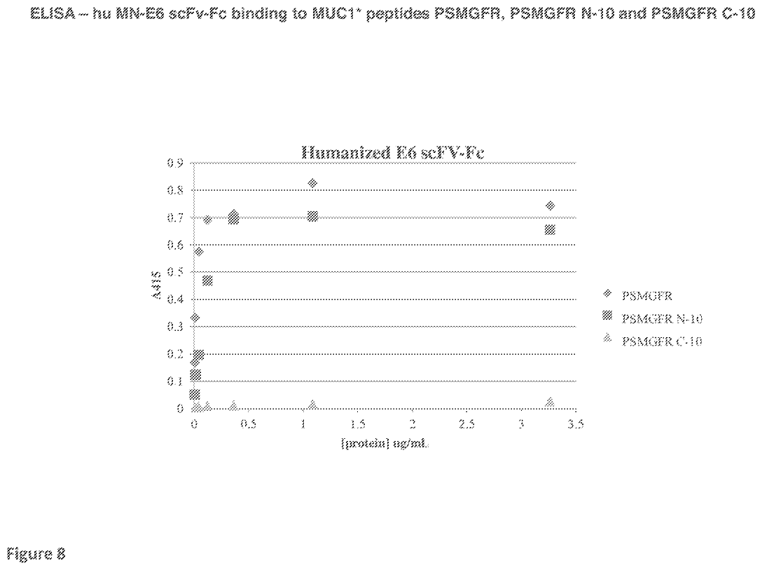

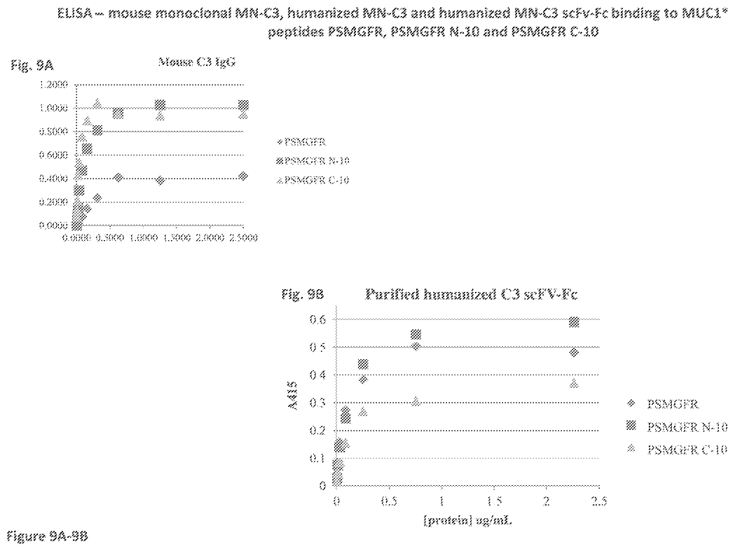

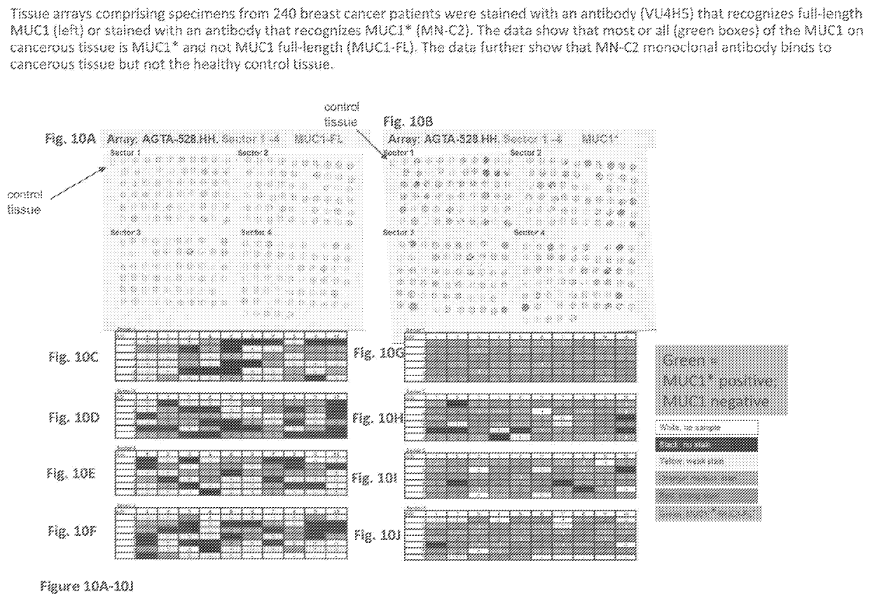

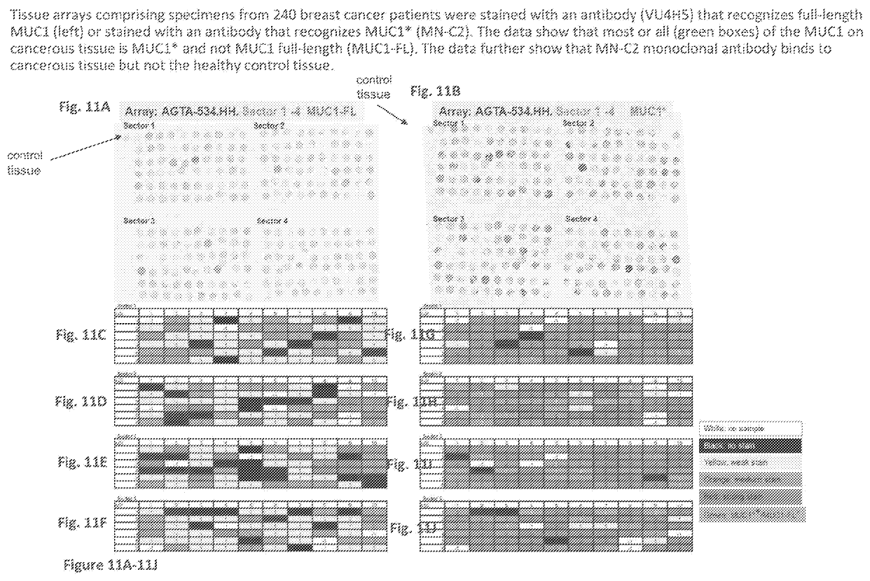

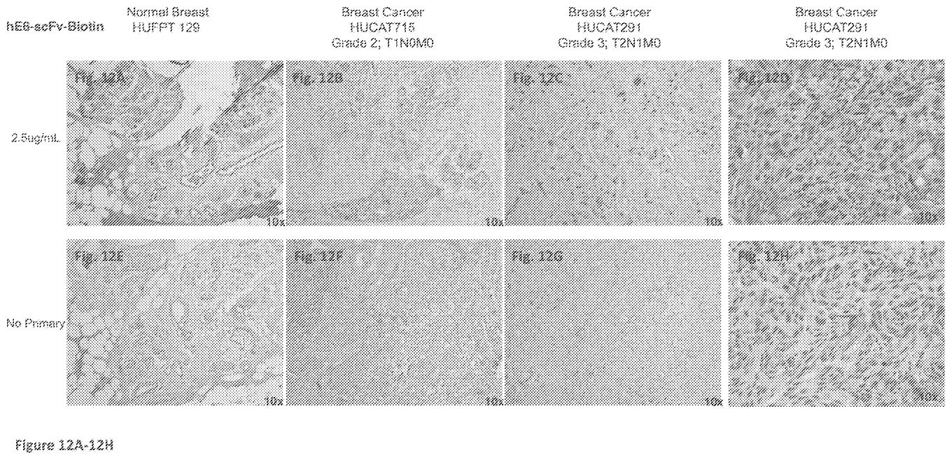

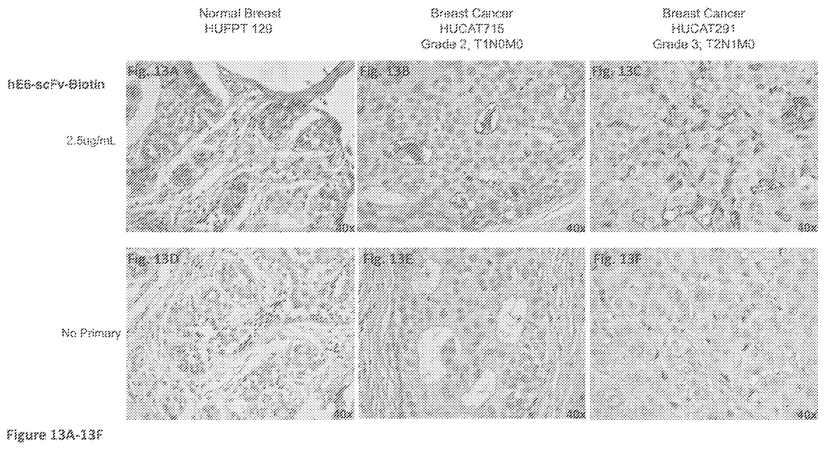

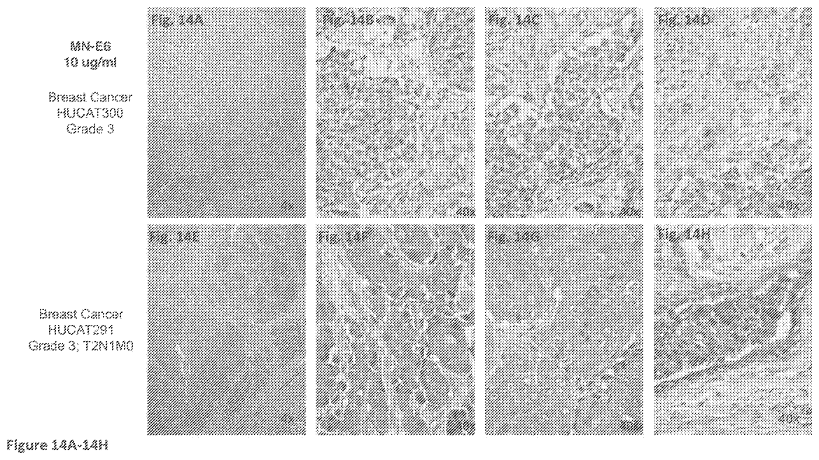

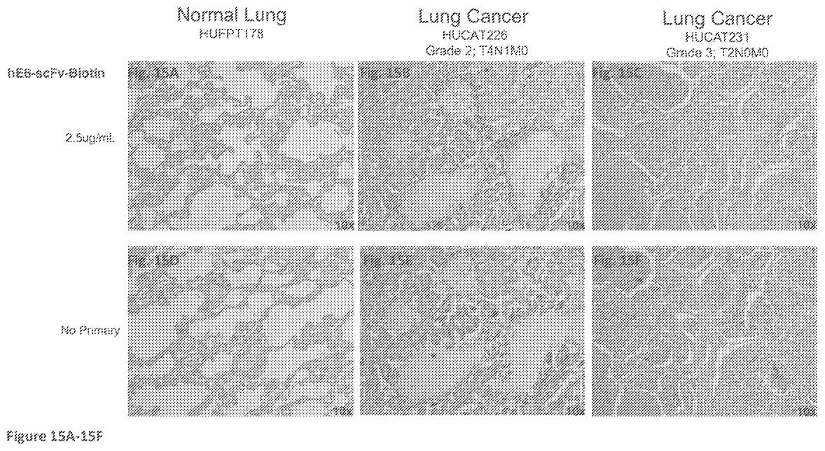

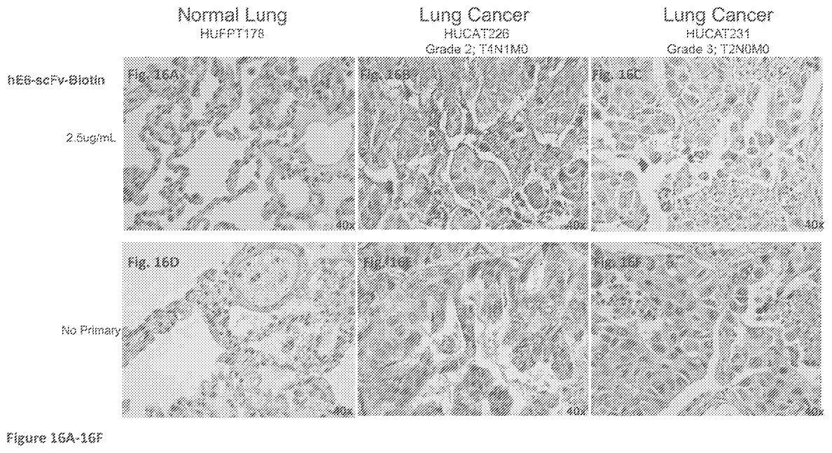

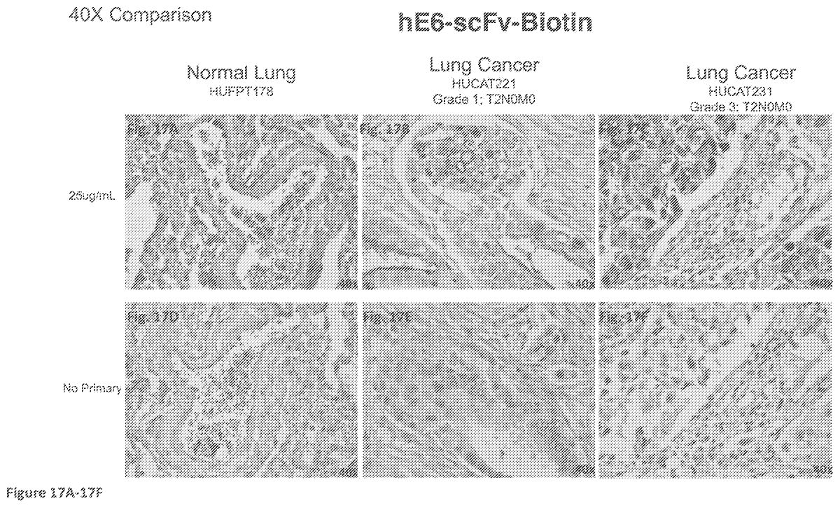

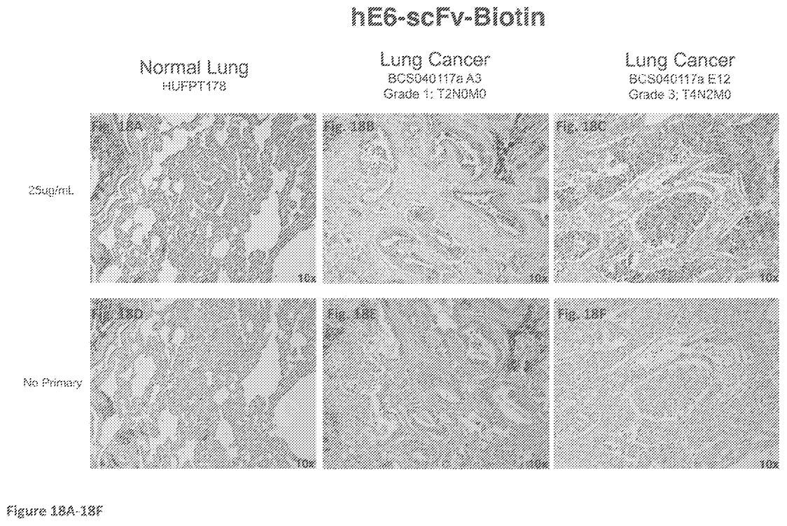

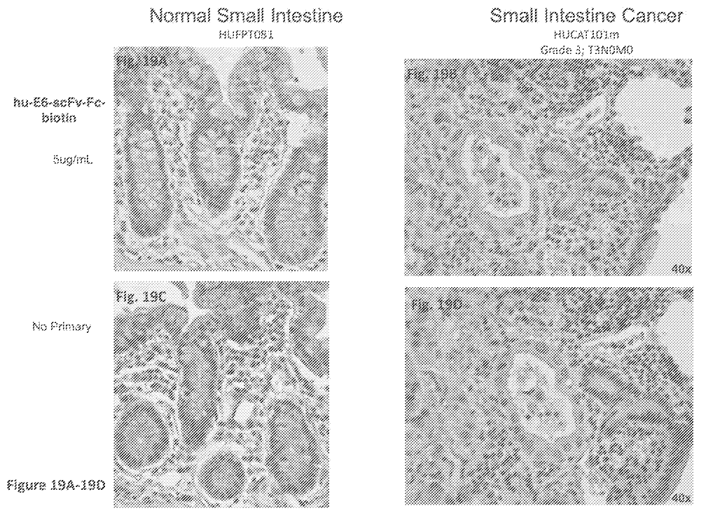

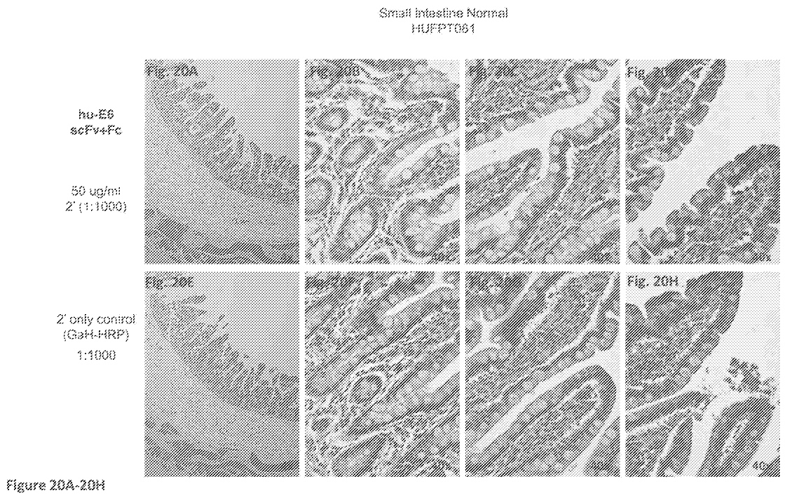

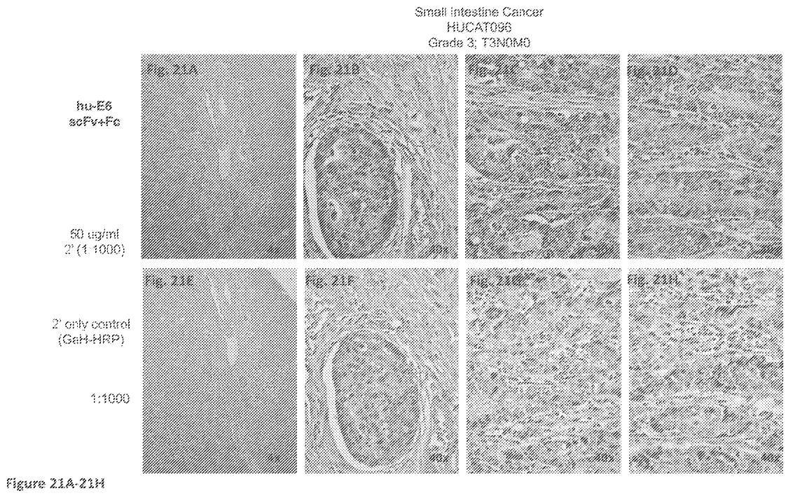

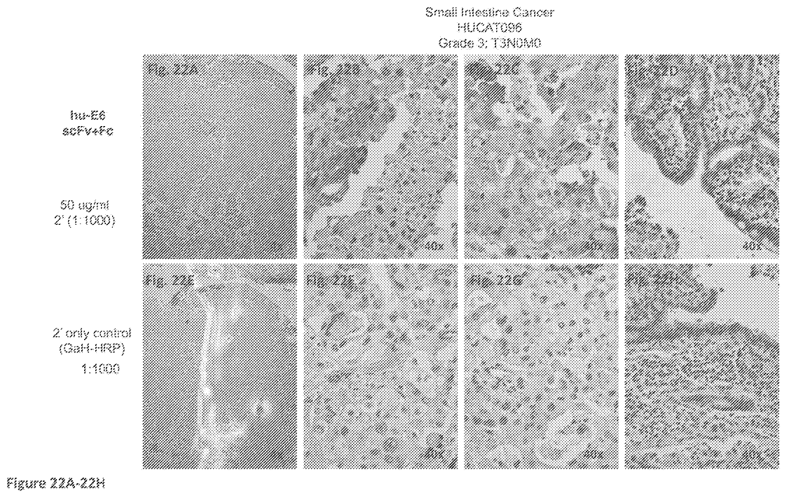

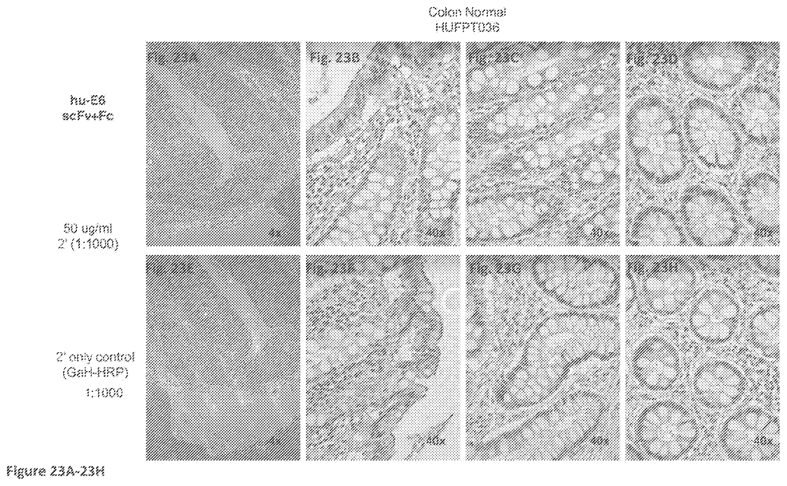

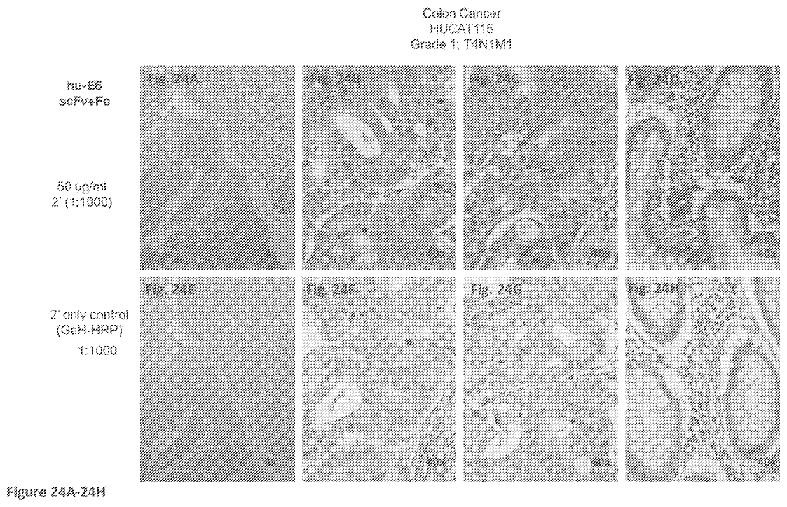

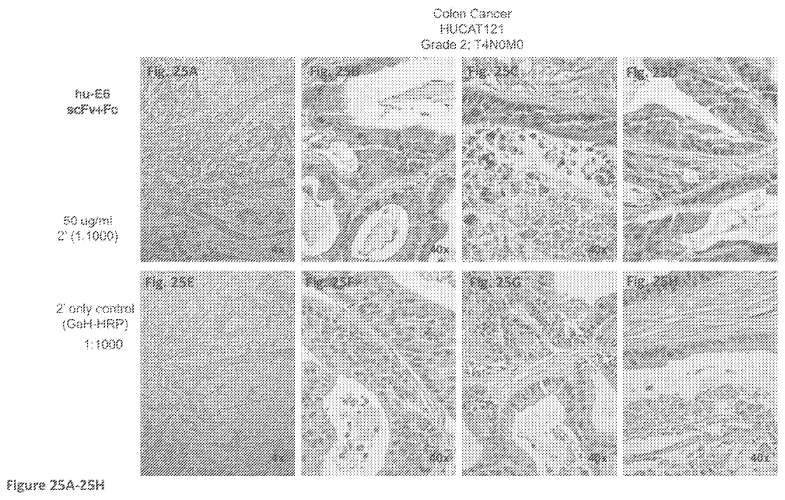

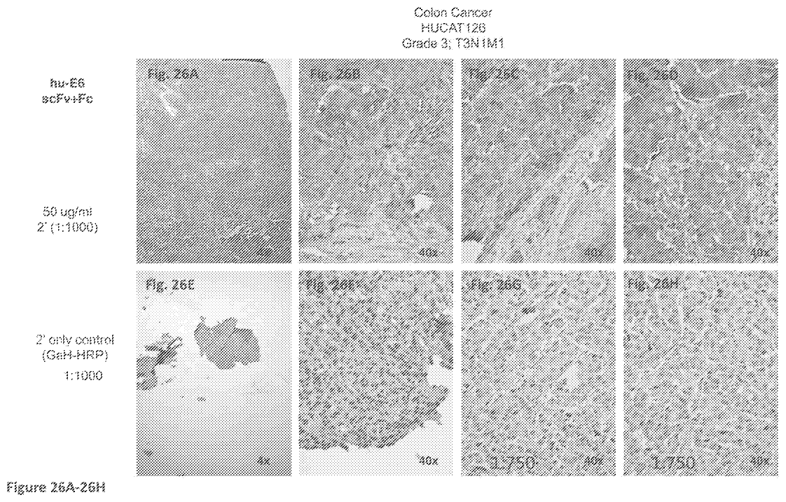

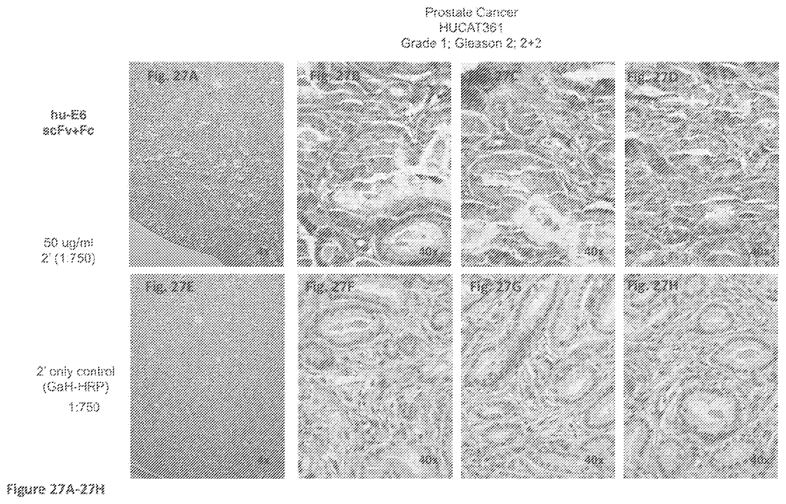

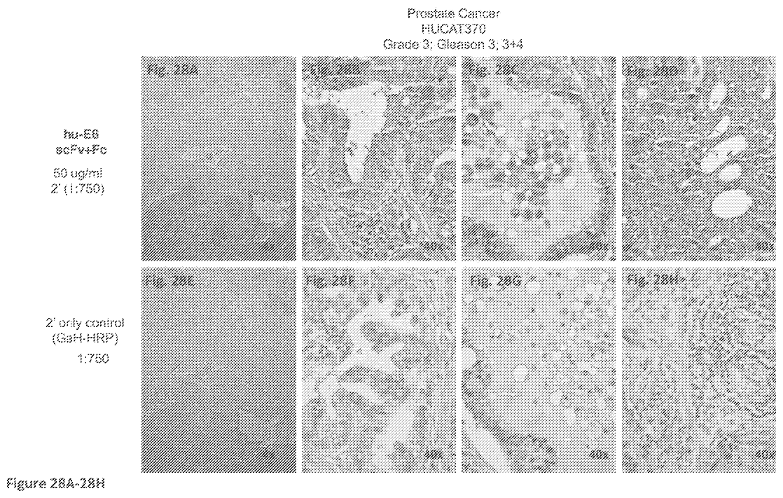

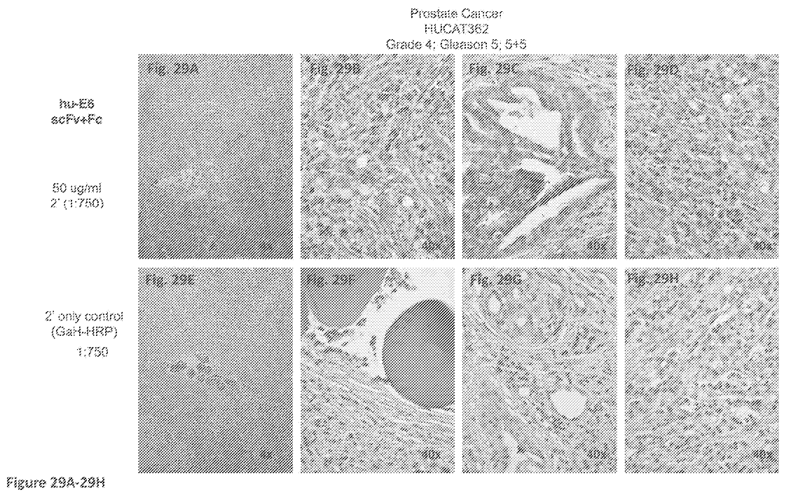

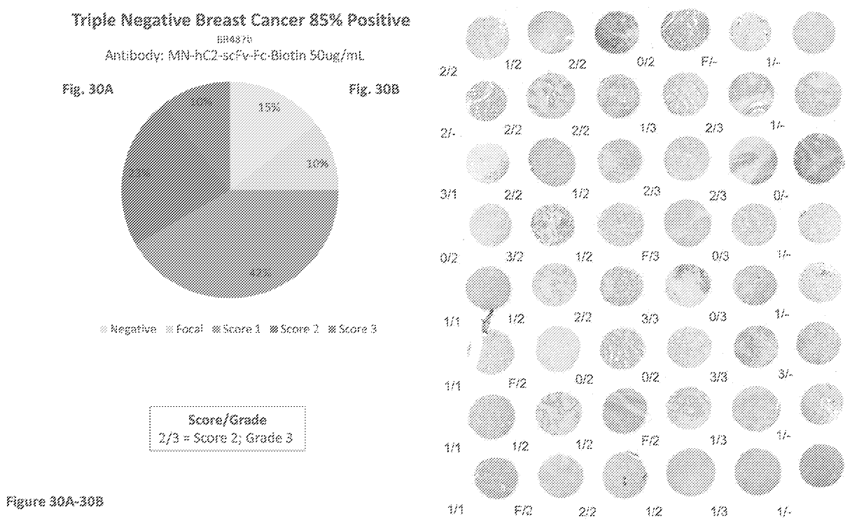

The most accurate way of demonstrating antibody specificity is testing the antibody on normal human tissue specimens compared to cancerous tissue specimens. MNC2 and MNE6 were previously shown to specifically bind to MUC1 or MUC1* positive cancer cells. Several breast tumor arrays were assayed using several anti-MUC1 or MUC1* antibodies. Essentially the studies involving serial sections of breast cancer tissue specimens from over 1,200 different breast cancer patients showed that very little full-length MUC1 remains on breast cancer tissues. The vast majority of the MUC1 expressed is MUC1* and is stained by MNC2. The analysis was performed by Clarient Diagnostics and tissue staining was scored using the Allred method. For example, FIG. 10 shows serial sections of breast cancer tissue arrays that were stained with either VU4H5, a commercially available anti-MUC1 antibody that binds to the tandem repeats, or MNC2 that binds to MUC1*. FIGS. 10 and 11 are photographs of breast cancer tissue arrays stained with either VU4H5 which recognizes MUC1-FL (full length) or MNC2 which recognizes cancerous MUC1*. Tissue staining was scored using Allred scoring method which combines an intensity score and a distribution score. Below the photographs of the tissue arrays are color-coded graphs displaying the results. As can be seen, the arrays stained with VU4H5 are very light and many tissues do not stain at all despite the published reports that MUC1 is aberrantly expressed on over 96% of all breast cancers as evidenced by nucleic acid based diagnostics. In contrast, the arrays stained with MNC2 are very dark (red versus yellow or white in graph). Additionally, many tissues did not stain at all with anti-full-length MUC1 but stained very dark with MNC2, (see green boxes in graph). Similarly, we stained normal or cancerous breast tissues with humanized MNE6 scFv-Fc. The antibody fragment was biotinylated so it could be visualized by a secondary streptavidin based secondary. As can be seen in FIG. 12, hMNE6 scFv-Fc does not stain normal breast tissue but stains cancerous breast tissue. Further, the intensity and homogeneity of staining increases with tumor grade and/or metastatic grade of the patient (FIGS. 12-13). Similarly, hMNE6 scFv-Fc did not stain normal lung tissue but did stain lung cancer tissue (FIGS. 14-18) and the intensity and distribution of staining increased as tumor grade or metastatic grade increased. FIG. 19 shows photographs of normal small intestine and cancerous small intestine tissues stained with humanized MNE6-scFv-Fc biotinylated anti-MUC1* antibody at 5 ug/mL, then stained with a secondary streptavidin HRP antibody. A) is a normal small intestine tissue. B) is small intestine cancer from patient as denoted in the figure. C,D are photographs of the corresponding serial sections that were stained with the secondary antibody alone. FIG. 20 shows photographs of normal small intestine tissues stained with humanized MNE6-scFv-Fc anti-MUC1* antibody at 50 ug/mL, then stained with a secondary goat-anti-human HRP antibody. A-D are normal small intestine tissue. E-H are photographs of the corresponding serial sections that were stained with the secondary antibody alone. FIG. 21 shows photographs of cancerous small intestine tissues stained with humanized MNE6-scFv-Fc anti-MUC1* antibody at 50 ug/mL, then stained with a secondary goat-anti-human HRP antibody. A-D are cancerous small intestine tissue from a patient as denoted in figure. E-H are photographs of the corresponding serial sections that were stained with the secondary antibody alone. FIG. 22 shows photographs of cancerous small intestine tissues stained with humanized MNE6-scFv-Fc anti-MUC1* antibody at 50 ug/mL, then stained with a secondary goat-anti-human HRP antibody. A-D are cancerous small intestine tissue from a patient as denoted in figure. E-H are photographs of the corresponding serial sections that were stained with the secondary antibody alone. FIG. 23 shows photographs of normal colon tissues stained with humanized MNE6-scFv-Fc anti-MUC1* antibody at 50 ug/mL, then stained with a secondary goat-anti-human HRP antibody. A-D are normal colon. E-H are photographs of the corresponding serial sections that were stained with the secondary antibody alone. FIG. 24 shows photographs of colon cancer tissues stained with humanized MNE6-scFv-Fc anti-MUC1* antibody at 50 ug/mL, then stained with a secondary goat-anti-human HRP antibody. A-D are colon cancer tissue from a metastatic patient as denoted in figure. E-H are photographs of the corresponding serial sections that were stained with the secondary antibody alone. FIG. 25 shows photographs of colon cancer tissues stained with humanized MNE6-scFv-Fc anti-MUC1* antibody at 50 ug/mL, then stained with a secondary goat-anti-human HRP antibody. A-D are colon cancer tissue from a Grade 2 patient as denoted in figure. E-H are photographs of the corresponding serial sections that were stained with the secondary antibody alone. FIG. 26 shows photographs of colon cancer tissues stained with humanized MNE6-scFv-Fc anti-MUC1* antibody at 50 ug/mL, then stained with a secondary goat-anti-human HRP antibody. A-D are colon cancer tissue from a metastatic patient as denoted in figure. E-H are photographs of the corresponding serial sections that were stained with the secondary antibody alone. FIG. 27 shows photographs of prostate cancer tissues stained with humanized MNE6-scFv-Fc anti-MUC1* antibody at 50 ug/mL, then stained with a secondary goat-anti-human HRP antibody. A-D are prostate cancer tissue from a patient as denoted in figure. E-H are photographs of the corresponding serial sections that were stained with the secondary antibody alone. FIG. 28 shows photographs of prostate cancer tissues stained with humanized MNE6-scFv-Fc anti-MUC1* antibody at 50 ug/mL, then stained with a secondary goat-anti-human HRP antibody. A-D are prostate cancer tissue from a patient as denoted in figure. E-H are photographs of the corresponding serial sections that were stained with the secondary antibody alone. FIG. 29 shows photographs of prostate cancer tissues stained with humanized MNE6-scFv-Fc anti-MUC1* antibody at 50 ug/mL, then stained with a secondary goat-anti-human HRP antibody. A-D are prostate cancer tissue from a patient as denoted in figure. E-H are photographs of the corresponding serial sections that were stained with the secondary antibody alone.

One aspect of the invention is a method for treating a patient diagnosed with, suspected of having, or at risk of developing a MUC1 positive or MUC1* positive cancer, wherein a specimen is obtained from the patient's cancer and is tested for reactivity with an antibody that binds to PSMGFR SEQ ID NO:2, or more specifically to the N-10 peptide (SEQ ID NO:3), or yet more specifically binds to N-10 peptide (SEQ ID NO:3), but does not bind to C-10 peptide (SEQ ID NO:825). The patient is then treated with an scFv, scFv-Fc or CAR T that comprises antibody variable fragments from the antibody that reacted with their cancer specimen or can be chosen from among MNC2, MNE6, 20A10, 3C2B1, 5C6F3, 25E6, 18G12, 28F9, 1E4, B12, B2, B7, B9, 8C7F3, or H11. Another aspect of the invention is a method for treating a patient diagnosed with, suspected of having, or at risk of developing a MUC1 positive or MUC1* positive cancer, wherein a specimen is obtained from the patient's cancer and is tested for reactivity with MNC2, MNE6, 20A10, 3C2B1, 5C6F3, 25E6, 18G12, 28F9, 1E4, B12, B2, B7, B9, 8C7F3, or H11; the patient is then treated with the antibody, antibody fragment, scFv, scFv-Fc-mut, BiTE or CAR T that comprises portions of the antibody that reacted with their cancer specimen.

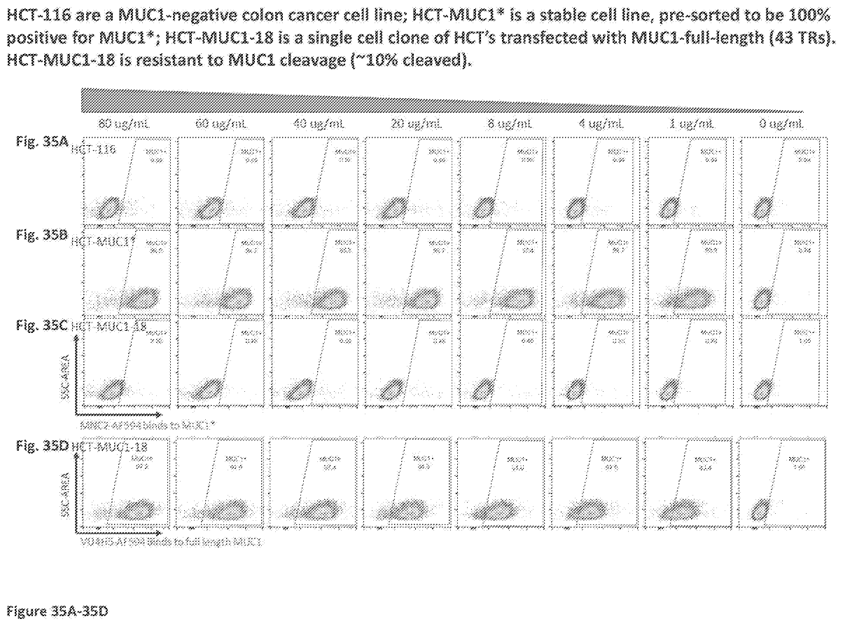

As we previously reported, it is MUC1*, the transmembrane cleavage product, not full-length MUC1, the is a growth factor receptor that drives tumor growth. The growth factors that activate MUC1* bind to ectopic sites that are only exposed after cleavage and release of the tandem repeat portion of MUC1. Antibodies of the invention, like the activating growth factors, cannot bind to full-length MUC1. FACS analysis clearly shows that anti-MUC1* antibody MNC2 is unable to bind to HCT-116, MUC1 negative cells (FIG. 35A), binds robustly to those cells if they are transfected with MUC1* (FIG. 35B), but will not bind to HCT cells transfected with full-length MUC1 (FIG. 35C). A commercially available anti-tandem repeat antibody VU4H5 clearly recognizes full-length MUC1 (FIG. 35D).

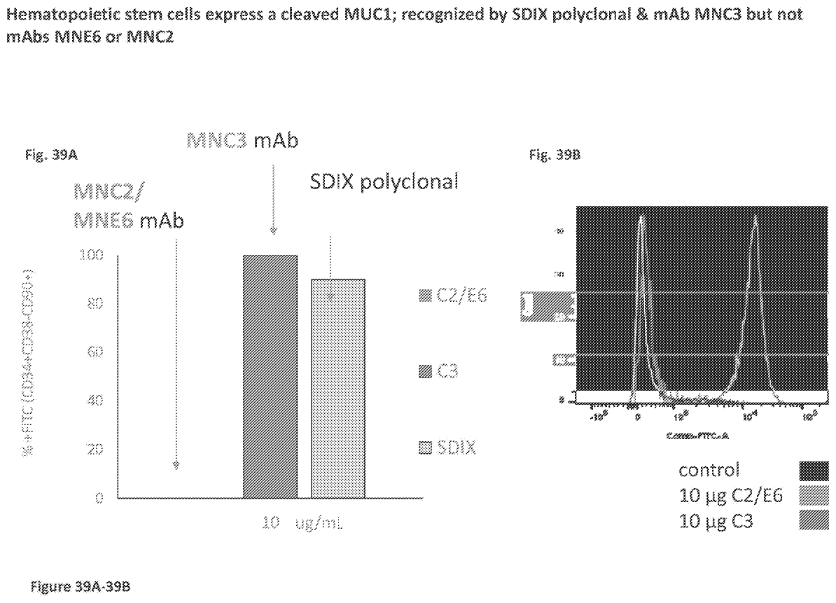

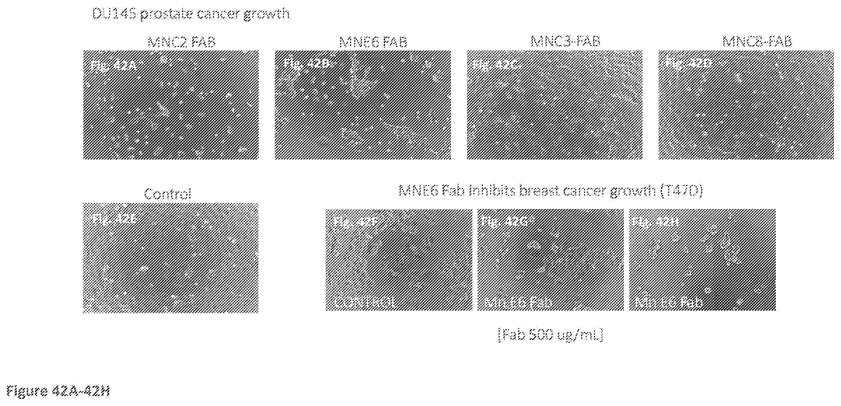

We discovered that MUC1 can be cleaved to MUC1* by more than one cleavage enzyme and that the site of cleavage affects its fold and consequently affects which monoclonal antibody is able to recognize that form of MUC1*. Different cancer cells or cancerous tissues express different cleavage enzymes. We tested various cleavage enzyme inhibitors on different cancer cell lines and found that an inhibitor that inhibits cleavage of MUC1 in one cancer cell line did not inhibit its cleavage in another cancer cell line. Similarly, PCR experiments showed that cleavage enzymes are expressed at different levels in different cells or cell lines. For example, hematopoietic stem cells of the bone marrow express a MUC1* that is recognized by monoclonal antibody MNC3 but not MNE6 or MNC2 (FIG. 39). The growth of DU145 prostate cancer cells and T47D breast cancer cells is inhibited by the Fabs of MNC2 and MNE6 but not by the Fabs of MNC3 or MNC8, indicating that the cancer cell lines express a MUC1* that is recognized by MNE6 and MNC2 but not by MNC3 or MNC8 (FIG. 42). PCR experiments show that CD34 positive cells of the bone marrow express about 2,500-times more MMP2 and about 350-times more ADAM28 than T47D breast cancer cells, while DU145 prostate cancer cells express about 2,000-times more ADAM TS16, about 400-times more MMP14 and about 100-times more MMP1 than T47D breast cancer cells (FIG. 43 and FIG. 44). Conversely, T47D breast cancer cells express about 80-times more MMP9 than the bone marrow cells and about twice as much as DU145 prostate cancer cells. Various cleavage enzyme inhibitors were tested for their ability to inhibit cleavage in different kinds of cancer cells.

General Strategy for Using Antibodies, Antibody Fragments and CARs that Target the Extracellular Domain of MUC1*

In one aspect of the invention, a second factor, which may be a cleavage enzyme, an antibody, a cytokine, or a second CAR, and a CAR are transduced into the same T cell. In another aspect of the invention, the second factor is on an inducible promoter such that its expression is activated when the CAR engages the targeted cancer cells. In some cases, the expression of the second factor is controlled by an inducible promoter. In one aspect of the invention, expression of the second factor is induced when the immune cell is activated, for example when it recognizes or engages its target. In one example, a T cell is transfected or transduced with a second factor whose expression is induced when the T cell recognizes a target cancer cell. One way to do this is to induce expression of the second factor when, or shortly after, an NFAT protein is expressed or translocated to the nucleus. For example, a sequence derived from an NFAT promoter region is put upstream of the gene for the second factor. In this way, when the transcription factors that bind to the promoter of the NFAT protein are present in sufficient concentration to bind to and induce transcription of the NFAT protein, they will also bind to that same promoter that is engineered in front of the sequence for transcription of the second factor. The NFAT protein may be NFAT1 also known as NFATc2, NFAT2 also known as NFATc or NFATc1, NFAT3 also known as NFATc4, NFAT4 also known as NFATc3, or NFAT5. In one aspect of the invention, the NFAT is NFATc1, NFATc3 or NFATc2. In one aspect of the invention, the NFAT is NFAT2 also known as NFATc1. SEQ ID NO:646 shows nucleic acid sequence of the upstream transcriptional regulatory region for NFAT2. The promoter sequence for NFAT gene may include the nucleic acid sequence of SEQ ID NO:781-783 or SEQ ID NO:815 as examples, but it can be seen that the optimal sequence or minimal sequence for expression of the second factor may be obtained by making fragments, extensions or mutations of the promoter and testing for the strength of the promoter with respect to expression of the second factor. In one aspect of the invention, the transcriptional regulatory region for NFAT2 is engineered upstream of the gene encoding the second factor, which if for cleavage enzyme MMP9 (SEQ ID NO:647) or the catalytic sub-unit of MMP9 (SEQ ID NO:648). In one aspect of the invention, the NFAT is NFATc3 and the promoter sequence of NFATc3 includes nucleic acid sequences from SEQ ID NO:816. In one aspect of the invention, the transcriptional regulatory region for NFATc3 is engineered upstream of the gene encoding the second factor, here as an example is MMP9. In another aspect of the invention, the NFAT is NFATc2. SEQ ID NO:817-818 shows nucleic acid sequence of the upstream transcriptional regulatory region for NFATc2. In one aspect of the invention, the transcriptional regulatory region for NFATc2 is engineered upstream of the gene encoding the second factor, which may be cleavage enzyme MMP9 (SEQ ID NO:647) or the catalytic sub-unit of MMP9 (SEQ ID NO:648).

Another method for having the expression of the second factor induced when the T cell or CAR T cell is activated is to have the gene for the second factor on an inducible promoter where the NFAT protein itself binds to and induces transcription of the second factor. In this case, an NFAT response element (NFAT RE) may be positioned upstream of the gene for the second factor or fragment of the second factor. The NFAT may bind to its responsive element upstream of the second factor alone or as part of a complex. The NFAT protein may be NFATc1, NFATc2, NFATc3, NFATc4, or NFAT5. In a preferred embodiment, the NFAT protein is NFAT2 aka NFATc1, aka NFATc. The gene of the second factor or fragment thereof is cloned downstream of an NFAT-response element (SEQ ID NO:649), which may be repeats of the response element (SEQ ID NO:650) and CMV minimal promoter (mCMV) (SEQ ID NO:651) to induce expression of second factor by NFAT protein. The NFAT response element may include nucleic acid sequence of NFAT consensus sequence (SEQ ID NO:804). The NFAT response element may include the nucleic acid sequence of SEQ ID NOS:805-814 as examples, but it can be seen that the optimal sequence or minimal sequence for expression of the second factor may be obtained by making fragments, extensions or mutations of the responsive element nucleic acid and testing for the strength of the responsive element with respect to expression of the second factor. The enhancer region of Foxp3 also contains NFAT response elements within the 120-bp from 2079 to 2098 (SEQ ID NO:821). The NFAT response element may include nucleic acid NFAT consensus sequence of (5′-cattttttccat-3′) (SEQ ID NO:819) or (5′-tttttcca-3′) (SEQ ID NO:820), which NFATc1 specifically binds to (Xu et al., Closely related T-memory stem cells correlate with in vivo expansion of CAR. CD19-T cells and are preserved by IL-7 and IL-15, Blood 2014 123:3750-3759), or repeats thereof. The NFAT response elements may also be separated by nucleic acid spacer sequences. Other NFAT responsive elements may exist and may further be discovered, and a skilled artisan in the art when directed to determine NFAT responsive element may do so by carrying out molecular biological assays to obtain it given the guidance of at least the responsive elements as set forth as SEQ ID NOS: 804-814 albeit as only mere examples. In one aspect of the invention, the cleavage enzyme that is downstream of the NFAT-response element and CMV minimal promoter is MMP9 (SEQ ID NO:652). In another aspect of the invention, the cleavage enzyme is a catalytic sub-unit of MMP9 (SEQ ID NO:653).

Because NFATs 1-4 are regulated by the calcineurin pathway, potential toxicities that may arise in a patient can be stopped by treatment with an immunosuppressive agent such as FK506, Cyclosporin, Cyclosporin A, or Tacrolimus that block calcineurin activity and inhibit NFAT translocation to the nucleus. The T cell transduced or transfected with a cleavage enzyme on an inducible promoter may also be transfected or transduced with a CAR that recognizes a protein or molecule on the cancer cell. In a specific example, the cleavage enzyme is one that is able to cleave MUC1 full-length and the CAR bears an antibody fragment that directs it to MUC1* on the surface of cancer cells.

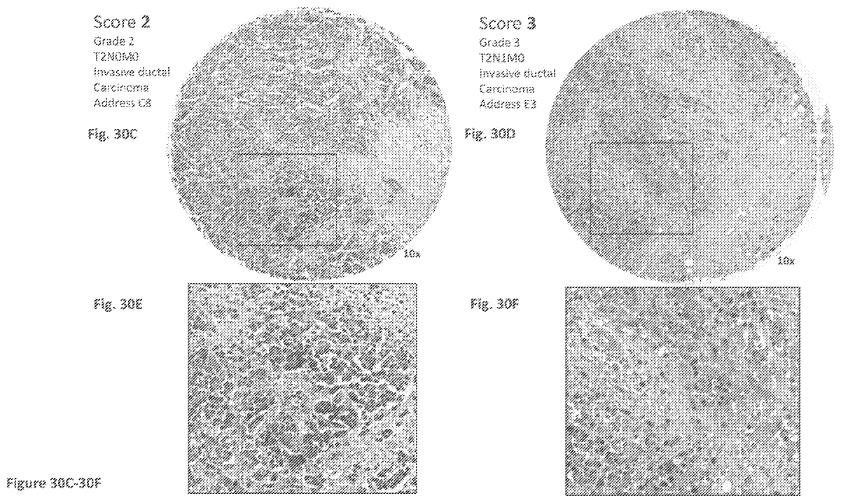

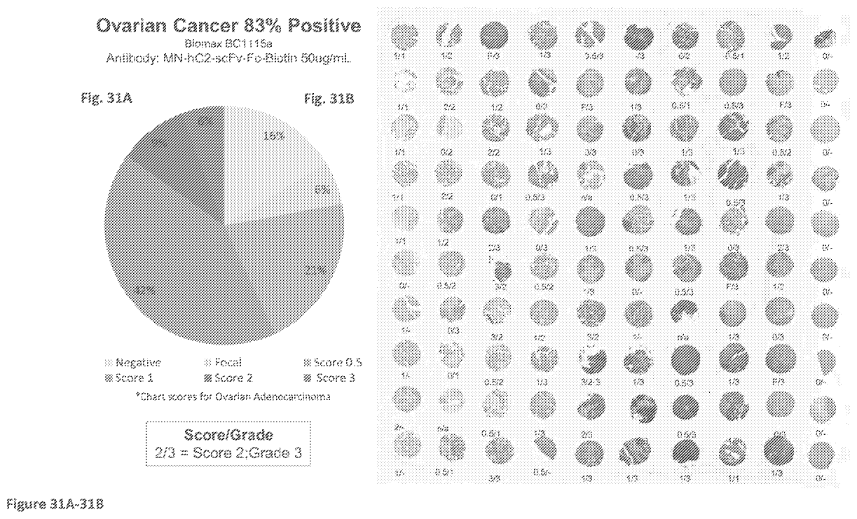

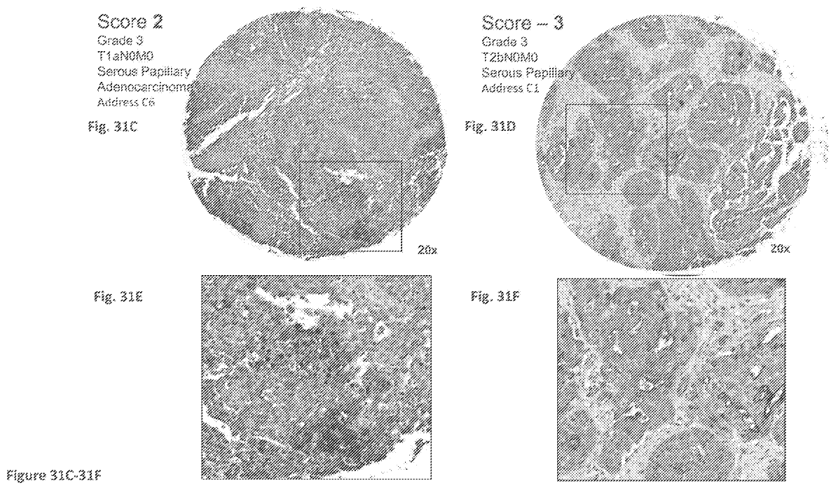

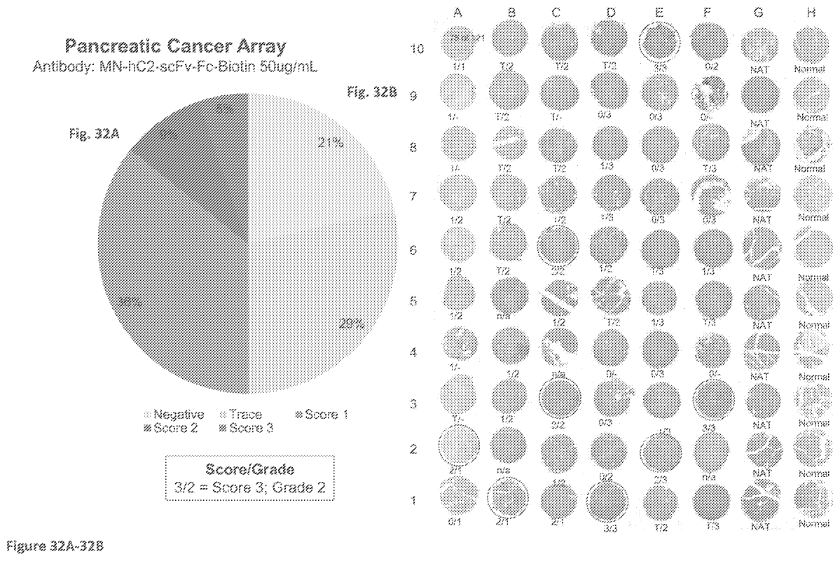

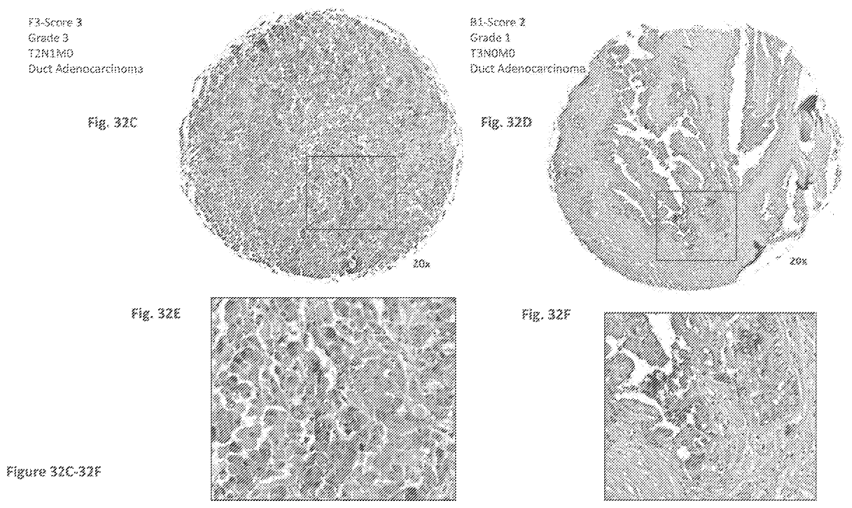

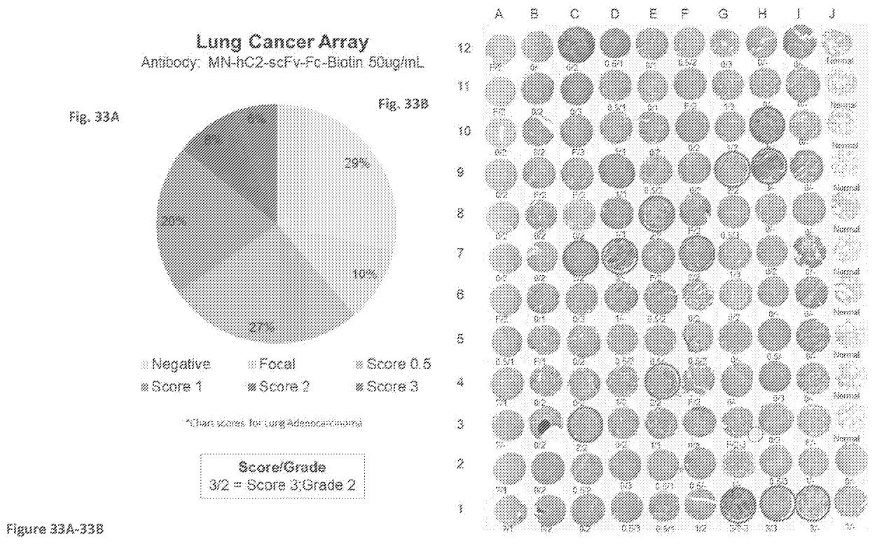

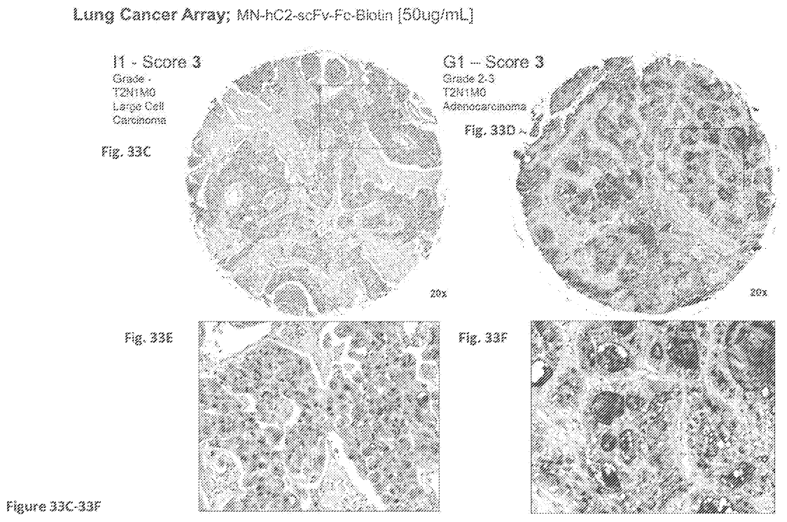

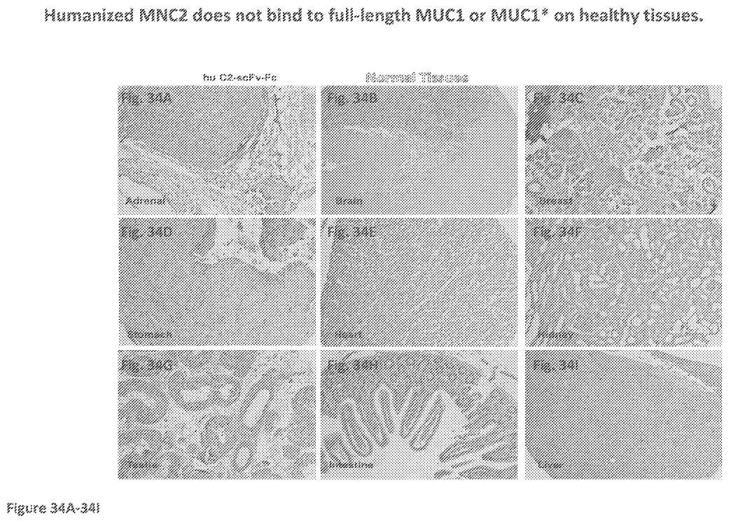

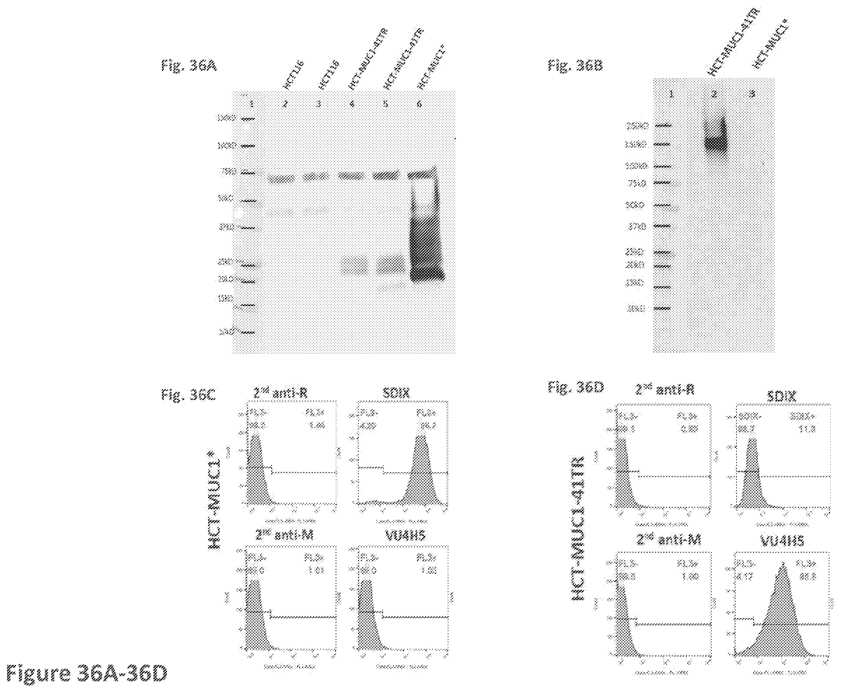

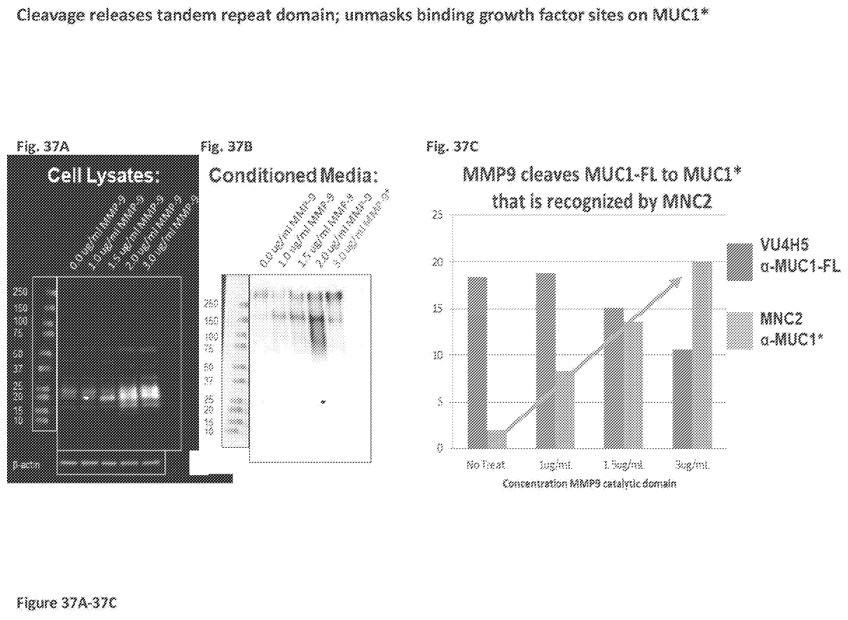

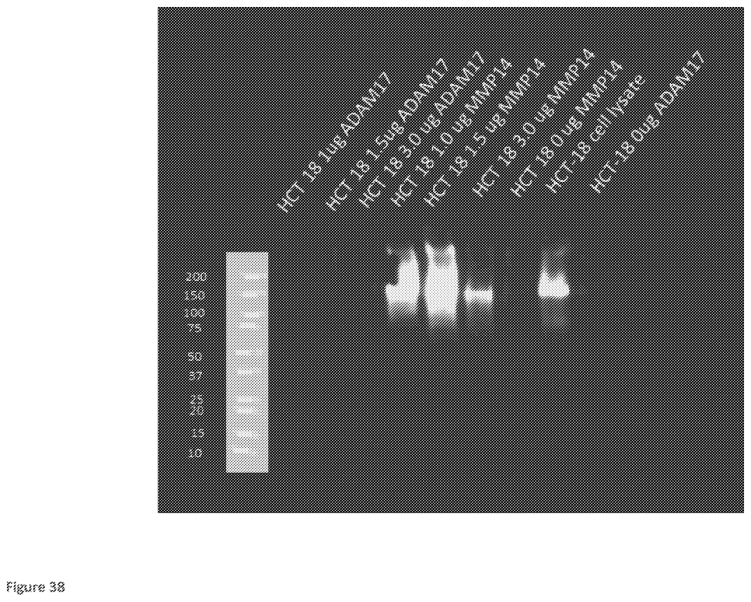

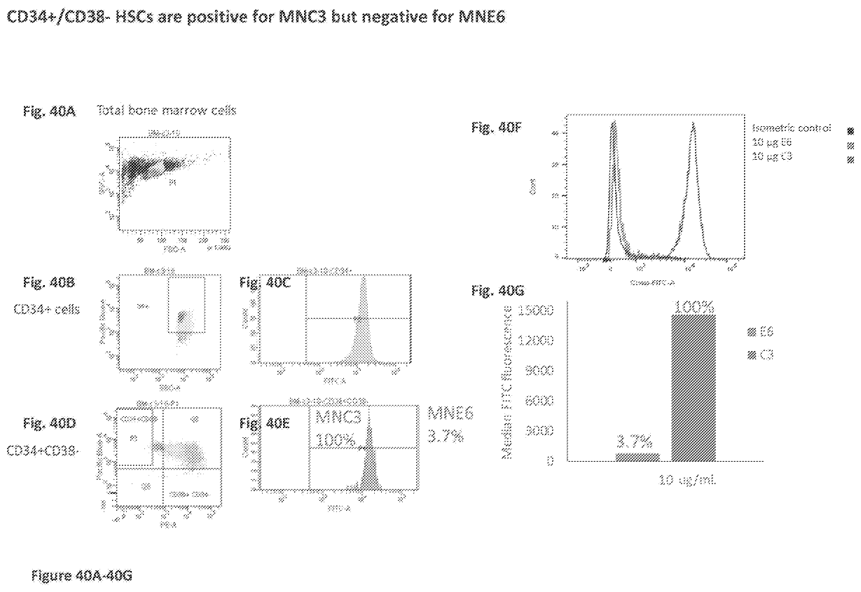

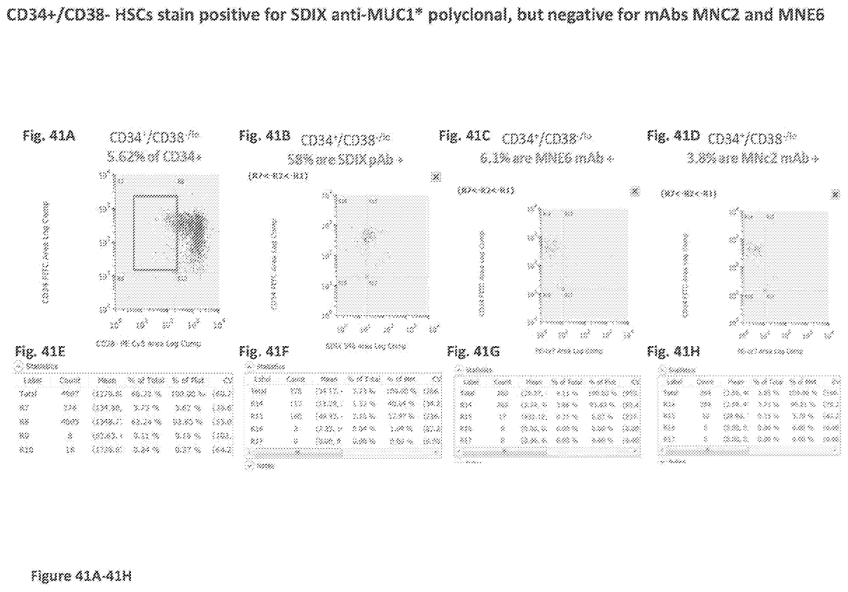

To determine which cleavage enzymes cleave MUC1 on cancer cells, we tested a series of MMP and ADAM enzyme inhibitors. These experiments pointed to MMP9 as being an important cleavage enzyme in cancer cells. To confirm that MMP9 cleaves MUC1 on cancer cells, we transfected HCT-116 MUC1 negative colon cancer cells with a mimic of full-length MUC1 having 41 tandem repeat domains: HCT-MUC1-41TR. Through single cell cloning we were able to establish this cell line wherein MUC1 only minimally gets cleaved to MUC1*. FIGS. 36A-36D show Western blots and FACS analysis showing that HCT-MUC1-41TR is 95% positive for full-length MUC1 and only 5-10% positive for the cleaved form, MUC1*. HCT-MUC1-41TR cells were incubated with MMP9 at varying concentrations and then assayed by immunofluorescence to measure binding of MNC2 monoclonal antibody to the resultant cells. As can be seen in FIGS. 37A-37C binding of MNC2 increased as the concentration of MMP9 added to the cells increased. These experiments show that MMP9 cleaves MUC1 to a form that is recognized by MNC2. The human cancer tissue array studies we performed (FIG. 30A-30F, FIG. 31A-31F, FIG. 32A-32F, FIG. 33A-33F) show that MNC2 recognizes the form of cleaved MUC1 that is present on cancerous tissue but not on healthy cells or tissues (FIG. 34A-341). Importantly, MNC2 does not recognize the form of cleaved MUC1 that is expressed on healthy hematopoietic stem cells of the bone marrow (FIGS. 39-41).

In one aspect of the invention, an immune cell is transduced with both a CAR to target the immune cell to the tumor, and a cleavage enzyme. The CAR and the cleavage enzyme can be encoded on the same plasmid or on two different plasmids. In one aspect, the cleavage enzyme is on an inducible promoter. In another aspect, expression of the cleavage enzyme is induced by a protein that is expressed when the immune cell is activated. In one case, expression of the cleavage enzyme is induced by an NFAT protein. In another aspect, expression of the cleavage enzyme is induced by NFATc1. In another aspect, expression of the cleavage enzyme is induced when one of the NFAT proteins binds to an NFAT response element that is inserted upstream of the gene for the cleavage enzyme or a catalytically active fragment thereof. In one aspect, the cleavage enzyme is MMP9 or a fragment of MMP9 that is catalytically active.

In one aspect of the invention, the cleavage enzyme is MMP9 (SEQ ID NO:643). Some cleavage enzymes are naturally expressed as pro-enzymes that need to be activated. This can be accomplished by biochemical means, by expressing a co-enzyme that activates a cleavage enzyme or by engineering the enzyme in an activated form. The invention anticipates overcoming this problem by co-expressing the cleavage enzyme with its activator. In one aspect of the invention, the cleavage enzyme is MMP9 and the co-activator is MMP3. In another aspect of the invention, the cleavage enzyme is expressed in a form that is already active, for example by expressing a fragment of the cleavage enzyme that still has catalytic function. In one case, the cleavage enzyme is an MMP9 fragment that is catalytically active. One example of an MMP9 catalytic fragment is given as SEQ ID NO:645.

MMP9, which must be activated by MMP3, is overexpressed in a large percentage of solid tumors. Further, it is known that MNC2 anti-MUC1* monoclonal antibody recognizes MUC1 after it is cleaved by MMP9. The various breast, ovarian, pancreatic and lung cancer tissue arrays that were shown in FIGS. 30-33 were probed with MNC2-scFv, further indicating that MUC1 in these cancers is being cleaved by MMP9. To see if cleavage of tumors by MMP9 would increase T cell access to the tumor, we did a series of experiments using a cell line that expresses full-length MUC1, HCT-MUC1-41TR, a breast cancer cell line that is a high expresser of both full-length MUC1 and MUC1* and a MUC1 negative cell line that we transfect with MUC1*45. We transfected cells with MMP9 and MMP3, which activates MMP9. We took the supernatant of those cells, which contained activated MMP9, and added it to the various cells, which were then co-cultured with T cells transduced with an anti-MUC1* CAR: huMNC2-CAR44. The result was greatly increased CAR T cell killing of the targeted MUC1/MUC1* positive cancer cells, compared to the control cells that were not incubated with a MUC1 cleavage enzyme.

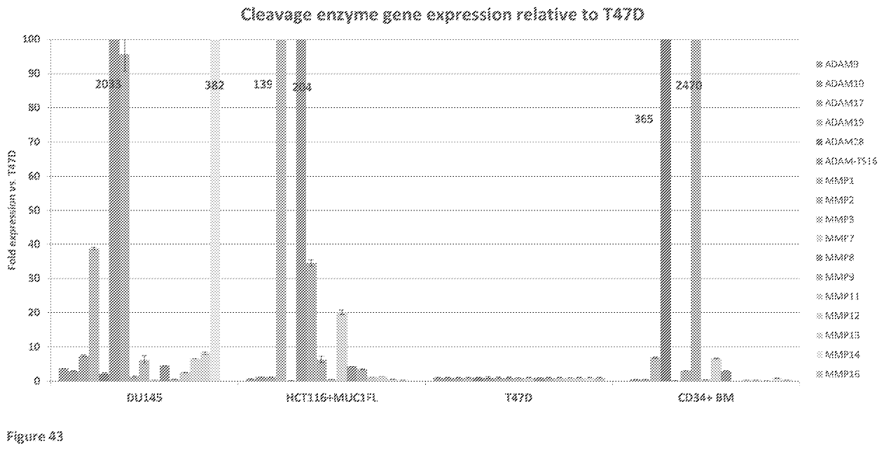

APMA is a biochemical that activates MMPs. We used APMA along with the conditioned media of cells that we transfected with either MMP9 or ADAM17 to see if any of these cleavage enzymes would cleave MUC1 on the HCT-MUC1-41TR cell line that only expresses full-length MUC1. As controls, we also tested the enzymes on HCT-MUC1* cells. The MUC1 and MUC1* expressing cells were stained with a red dye, CMTMR. Human T cells that were transduced with an anti-MUC1* CARs, CAR44 or CAR50 were co-cultured with the cancer cells. Untransduced T cells were used as a control (FIG. 45A-45P). As can be seen in FIG. 45B, FIG. 45C, and FIG. 45D, the anti-MUC1* CAR T cells effectively recognized and clustered the HCT-MUC1* cancer cells, which is a sign of T cell activation and killing. However, no CAR T cell induced clustering is visible in the wells containing HCT-MUC1-41TR, the full-length MUC1 expressing cells (FIG. 45F, FIG. 45G, and FIG. 45H). However, the cells that were incubated with activated MMP9 show dramatic increase in CAR T cell induced clustering (FIG. 45J, FIG. 45K, and FIG. 45L), indicating that MMP9 cleaved the full-length MUC1 to a form of MUC1* that is recognized by MNC2 monoclonal antibody and more specifically by huMNC2-scFv. ADAM17 had no apparent effect. ADAM17 either did not cleave MUC1 or cleaved it at a position that is not recognized by MNC2, which is more likely (FIG. 45N-45P).

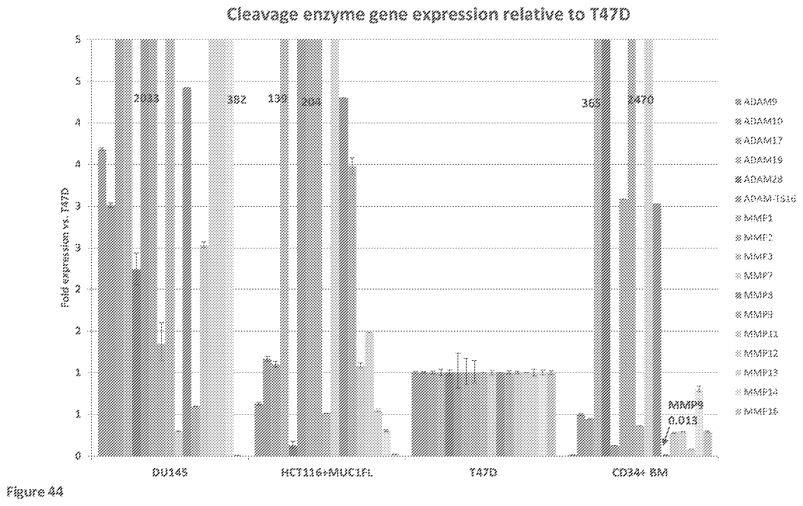

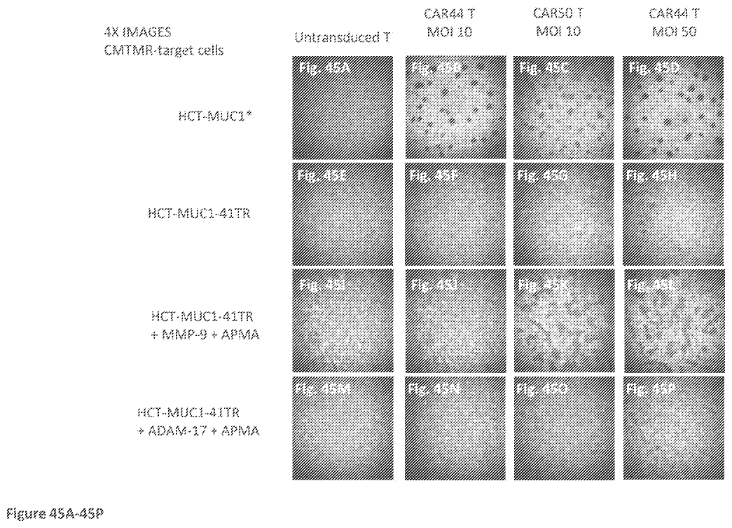

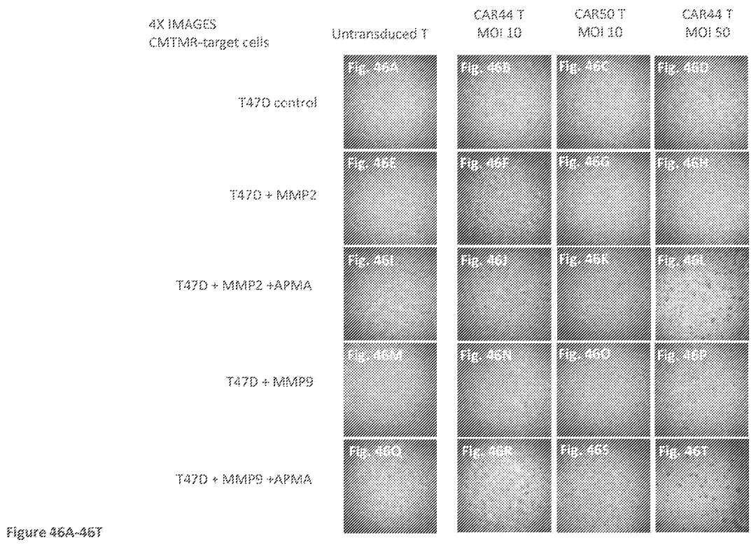

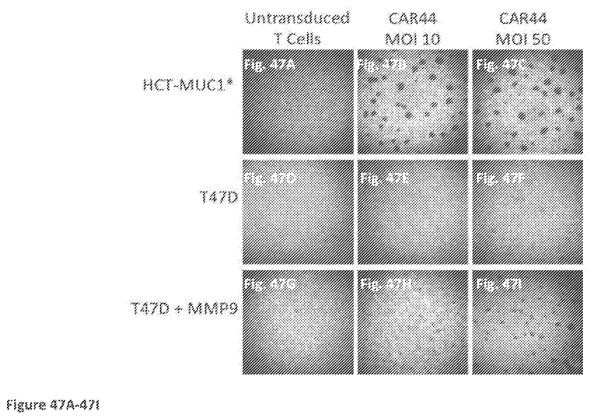

We performed the same experiment, this time using T47D breast cancer cells that were hard to kill using anti-MUC1* CAR T cells presumably because they express high levels of full-length MUC1 as well as MUC1* (FIG. 46A-46T). As can be seen in FIGS. 46B, 46C, and 46D, anti-MUC1* CAR44 and CAR50 have little effect on the T47D cancer cells. Only in FIG. 46D, which is CAR44 at the highest level of CAR expression in the T cells, do we see a small amount of CAR T cell induced clustering. However, the presence of activated MMP2 (FIG. 46J, 46K, 46L) or activated MMP9 (FIG. 46R, 46S, 46T) shows a dramatic increase in CAR T cell recognition, clustering and killing, showing that cleavage of full-length MUC1 increases T cell access to the cancer cells. To ensure that the addition of the APMA was not inducing cleavage or anti-MUC1* CAR T recognition by some other mechanism, we made a catalytically active form of MMP9 and added it to T47D cells that were then co-cultured with MNC2-CAR44 T cells (FIG. 47A-47I). As can be seen in the figure, MNC2-CAR T cells recognize and cluster cells transfected with MUC1* (FIG. 47B-47C), poorly cluster T47D breast cancer cells that express both full-length MUC1 and MUC1* (FIG. 47E-47F), but robustly bind to and cluster the T47D cells after the addition of the catalytically active MMP9 (FIG. 47H-47I). This results supports the claim that MNC2 does not recognize full-length MUC1 but does recognize the growth factor receptor MUC1*. Note that the full-length MUC1 expressed on this cell line may sterically hinder the binding of CAR T cells near the cell membrane.

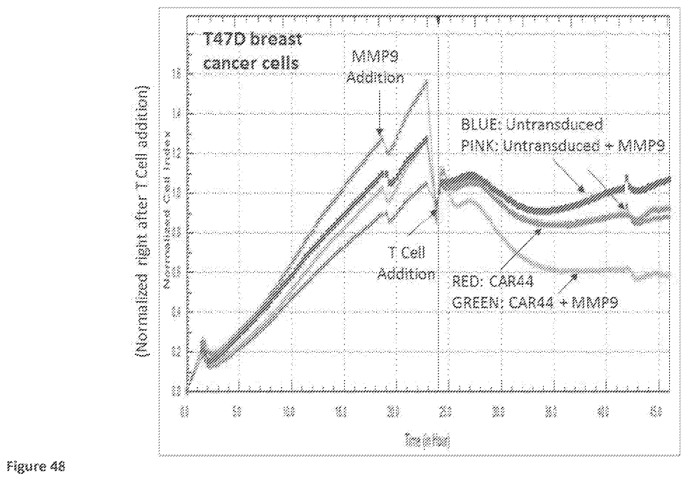

In another example, T47D MUC1 positive tumor cells were incubated with a recombinant catalytic domain of MMP9 (Enzo Life Sciences, Inc., Farmingdale, NY) at either 100 ng/mL or 500 ng/mL. Western blot analysis showed that the MUC1/MUC1* positive cancer cells underwent extensive cleavage of MUC1 to MUC1*. In another example, T47D breast cancer cells were pre-incubated with a human recombinant MMP9 catalytic domain protein then co-cultured with anti-MUC1* CAR44 T cells. The specific killing of the T47D cells by CAR44 T cells was monitored in real-time on an xCelligence instrument that measures impedance as a function of time. This analysis uses electrode arrays upon which cancer cells are plated. The adherent cancer cells insulate the electrode and cause an increase in impedance as they grow. Conversely, T cells are not adherent and remain in suspension so do not increase or decrease impedance. However, if the T cells or CAR T cells kill the cancer cells on the electrode plate, the cancer cells ball up and float as they die, which causes the impedance to decrease. The addition of MMP9 catalytic domain dramatically increased the killing of T47D cancer cells. FIG. 48 shows an xCelligence graph of T47D breast cancer cells in co-culture with either untransduced T cells, as a control, or huMNC2-CAR44 T cells over a 45 hour period. After 18 hours of cancer cell growth, a catalytic sub-unit MMP9 was added to some of the cells. At 25 hours, T cells were added. As can be seen, huMNC2-CAR44 T cell killing is greatly improved when the T47D cells are pre-incubated with cleavage enzyme MMP9. In the xCelligence system, target cancer cells, which are adherent, are plated onto electrode array plates. Adherent cells insulate the electrode and increase the impedance. The number of adherent cancer cells is directly proportional to impedance. T cells are not adherent and do not contribute to impedance. Therefore, increasing impedance reflects growth of cancer cells and decreasing impedance reflects killing of cancer cells. Prostate cancer cell line DU145 expresses both MUC1 and MUC1* but at a much lower level of expression than T47D cells. DU145 cells are efficiently killed by anti-MUC1* CAR T cells in the presence or absence of a cleavage enzyme.

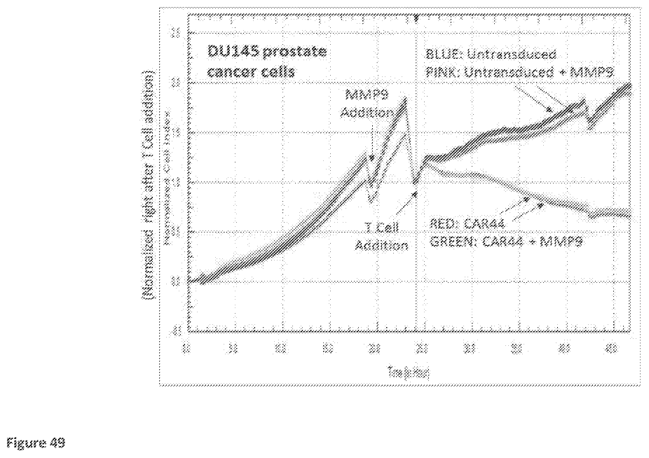

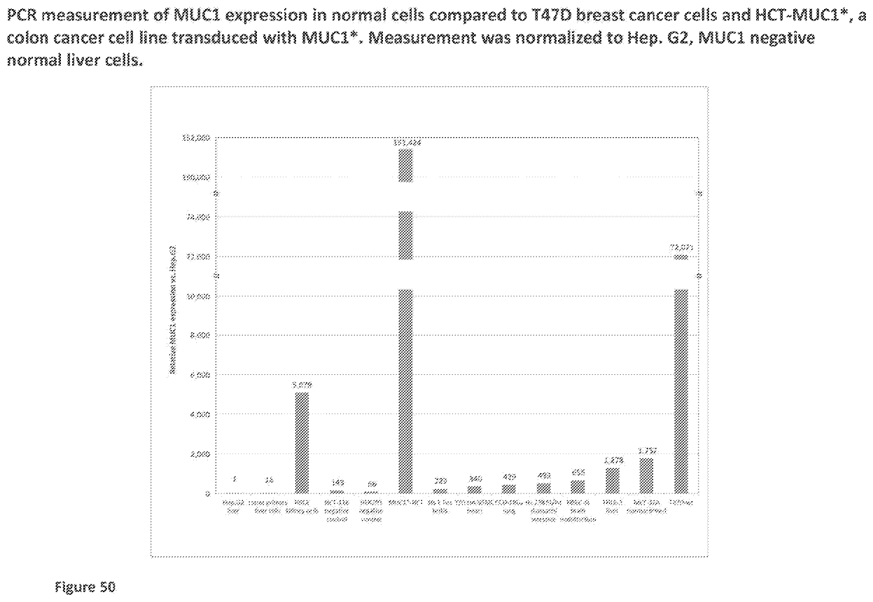

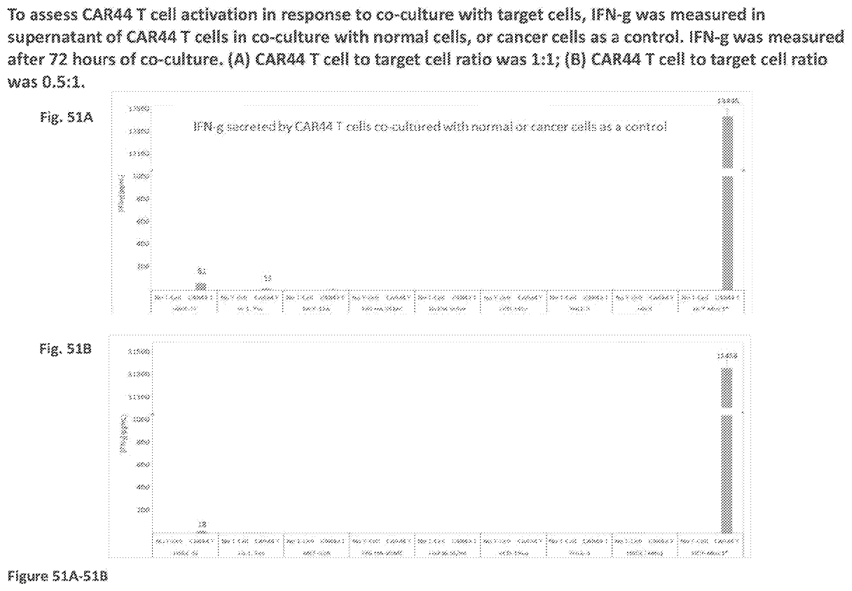

FIG. 49 shows an xCelligence graph of DU145 prostate cancer cells in co-culture with either untransduced T cells, as a control, or huMNC2-CAR44 T cells over a 45 hour period. After 18 hours of cancer cell growth, a catalytic sub-unit MMP9 was added to some of the cells. At 25 hours, T cells were added. As can be seen, huMNC2-CAR44 T cell killing of low density MUC1/MUC1* positive cancer cells is not affected by pre-incubation with cleavage enzyme MMP9. DU145 cancer cells express a significantly lower amount of MUC1 which includes the full-length form as well as MUC1*. The lower density of full-length MUC1 does not sterically hinder T cell access to the membrane proximal MUC1*. DU145 cells represent an early stage cancer that expresses both full length and cleaved MUC1 but at lower levels so that T cell access is not sterically hindered. T47D cells represent mid-stage cancers that express high levels of both MUC1 and MUC1*, wherein the density of MUC1 full-length sterically hinders access of T cells to the tumor. HCT-MUC1* cells are a MUC1 negative cell line that has been stably transfected with MUC1*45, and they represent late stage cancer cells. It is significant that MUC1 cleaved to MUC1* by MMP9 is recognized by the anti-MUC1* antibody MNC2, which is the targeting head of the CAR. Immune cell access to tumor antigens on the cancer cell surface can be sterically hindered by the presence of bulky extra cellular domain proteins or other obstructing elements also known as the tumor micro-environment. The aforementioned serve as an example that can be extended to improve the efficacy of CAR T therapies that target other tumor antigens. In one aspect of the invention, an immune cell is transfected or transduced with both a CAR comprising an antibody fragment that targets a tumor antigen and a cleavage enzyme. In another aspect of the invention, an immune cell is transfected or transduced with both a CAR comprising an antibody fragment that targets a tumor antigen and a cleavage enzyme that cleaves a tumor antigen to a form recognized by the antibody fragment of the CAR. In one aspect, an immune cell is transfected or transduced with both a CAR comprising an antibody fragment that targets a tumor antigen and a cleavage enzyme that cleaves a tumor antigen to a form recognized by the antibody fragment of the CAR, wherein the antibody fragment of the CAR recognizes MUC1* extra cellular domain and the cleavage enzyme cleaves MUC1 to MUC1*. In one aspect, an immune cell, which may be a T cell or an NK cell, is transfected or transduced with a CAR comprising an antibody fragment derived from MNC2, MNE6, MNC3 or MNC8 and a cleavage enzyme chosen from the group comprising MMP1, MMP2, MMP3, MMP7, MMP8, MMP9, MMP11, MMP12, MMP13, MMP14, MMP16, ADAM9, ADAM10, ADAM17, ADAM 19, ADAMTS16, ADAM28 or a catalytically active fragment thereof. In one aspect of the invention, the immune cell is derived from a stem cell that has been directed to differentiate into an immune cell type in vitro. In another aspect, a CAR containing sequences of the antibody are expressed in a stem cell, which then may be differentiated into an immune cell. In one case, the immune cell is a T cell. In another case, the immune cell is an NK cell.

In one aspect of the invention, a person diagnosed with cancer or at risk of developing cancer is administered a sufficient amount of an immune cell transduced with both a CAR and a cleavage enzyme. In another aspect of the invention, a person diagnosed with cancer or at risk of developing cancer is administered a sufficient amount of an immune cell transduced with both a CAR and a cleavage enzyme, wherein the cleavage enzyme is on an inducible promoter that is activated by proteins that are expressed when the immune cell becomes activated. In another aspect of the invention, a person diagnosed with cancer or at risk of developing cancer is administered a sufficient amount of an immune cell transduced with both a CAR and a cleavage enzyme, wherein the cleavage enzyme is on an inducible promoter that is activated by one or more NFAT. In one case the NFAT is NFATcT. In another aspect, the NFAT is NFATc3. In another aspect, the NFAT is NFATc2. In any of the instances above, the extra cellular domain of the CAR comprises a fragment of an anti-MUC1* antibody. In one aspect, the anti-MUC1* antibody is MNC2scFv or a humanized form of MNC2scFv. In another aspect, the anti-MUC1* antibody is MNE6scFv or a humanized form of MNE6scFv. In any of the instances above, the immune cell can be a T cell, an NK cell, a mast cell, or a dendritic cell. In one aspect the immune cell is derived from a stem cell that has been directed to differentiate to that immune cell type in vitro. In another aspect, a CAR containing sequences of the antibody are expressed in a stem cell, which then may be differentiated into an immune cell. In one case, the immune cell is a T cell. In another case, the immune cell is an NK cell.

It is not intended that the present invention be limited to one or two specific methods of having expression of a cleavage enzyme induced by an activated T cell. We have demonstrated specific expression of a cleavage enzyme only upon T cell activation by constructing a plasmid with the cleavage enzyme gene downstream of an NFAT promoter sequence or downstream of one or more repeats of NFAT response elements. In another aspect of the invention, expression of the cleavage enzyme is induced by constructing a plasmid where the cleavage enzyme gene is inserted downstream of an IL-2 promoter sequence or downstream of an IL-2 response element, then inserting the plasmid into an immune cell. In another aspect of the invention, expression of the cleavage enzyme is induced by constructing a plasmid where the cleavage enzyme gene is inserted downstream of a Calcineurin promoter sequence or downstream of a Calcineurin response element, then inserting the plasmid into an immune cell and then administering to a patient for the treatment or prevention of cancers. There are also drug-inducible plasmids that can be used to induce expression of the cleavage enzyme or used to stop expression induced by an element of an activated T cell. These drug inducible systems may include tetracycline-inducible systems, Tet-on, Tet-off, tetracycline response elements, doxycycline, tamoxifen inducible systems, ecdysone inducible systems and the like.

It is not intended that the present invention be limited to one or two specific promoters used in the plasmids encoding the CARs or inducible cleavage enzymes. As is known by those skilled in the art, many promoters can be interchanged including SV40, PGK1, Ubc, CAG, TRE, UAS, Ac5, polyhedron, CaMKIIa, GAL1, GAL10, TEF1, GDS, ADH1, CaMV35S, Ubi, H1 and U6.Another solution to the problem of steric hindrance of CAR T cell access, caused by bulky cell surface proteins such as MUC1-FL, is to increase the length of the linker region of the CAR that is expressed by the T cell. In standard design CARs, the length of the extracellular linker region between the transmembrane portion and the antibody fragment is about 45-50 amino acids in length. We made long-arm CARs where the length of the extracellular linker is extended from about 50 amino acids to 217-290 amino acids. Co-culture assays show that CARs with longer extracellular linkers have improved access to the tumor-associated antigen on the target cancer cells.

BiTEs, Bispecific Antibodies and Antibody-Like Molecules with Multi-Specificity

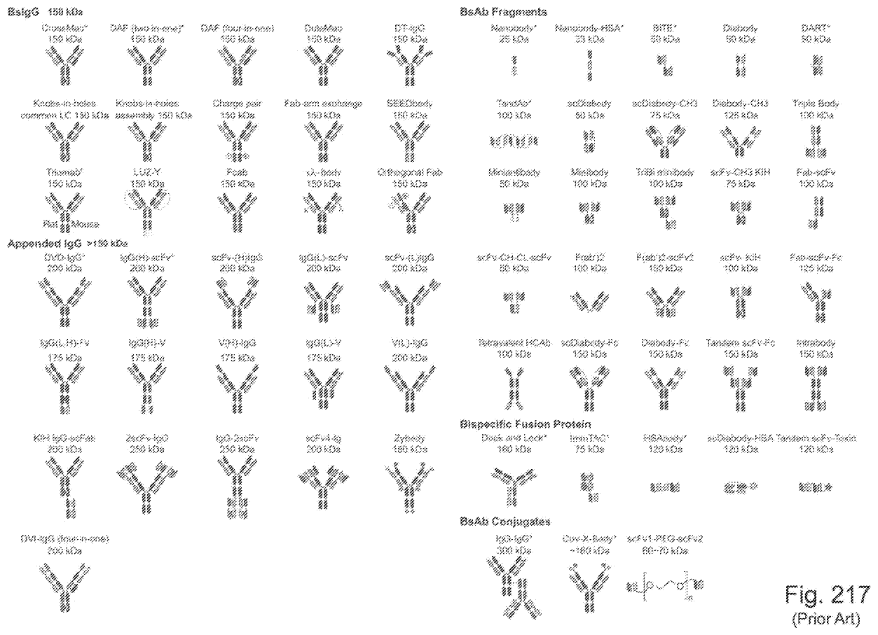

Antibodies of the invention can be incorporated into a molecule comprising at least two binding arms wherein at least two bind to different antigens. Several examples of multi-specific antibody-like molecules have been described (Brinkmann and Kontermann, MABS, Vol. 9, No. 2, 182-212 (2017); Spiess et al. Molecular Immunology 67, 95-106 (2015)), which references are incorporated by reference herein in their entirety, but in particular with respect to disclosures of making of bispecific antibodies and fragments of antibodies that are bispecific. It is also understood that various terminologies such as “CrossMab”, “DutaMab” found in FIG. 217 of the present application and FIG. 1 of Spiess et al. cited herein are terms of art and should be interpreted within the context of the description provided in Spiess et al. or Brinkmann and Kondermann cited herein.

The variable regions of antibodies of the invention, or fragments thereof, can be incorporated into molecular formats wherein one molecule is able to simultaneously bind to at least two different antigens. The stability of these various antibody-like molecules can be increased by the introduction of Cysteines for the formation of disulfide bonds. The stability of antibodies of the invention as well as of various antibody-like molecules, which may be multi-specific can be increased by the introduction of mutations as more fully described in Beckman, U.S. Pat. No. 9,708,388B2, which reference is incorporated by reference herein in its entirety, but in particular with respect to disclosures of making of bispecific antibodies and fragments of antibodies that are bispecific or multispecific. The invention contemplates incorporating variable regions, or fragments thereof, of antibodies of the invention in combination with other antibodies having other binding specificities, or fragments thereof, into a variety of antibody-like formats including but not limited to the following. Among the possible antibody-like formats are bispecific antibodies, which may or may not include an Fc region. Bispecific antibodies that contain at least one Fc region will have a higher molecular weight and an extended half-life in the body. In a variation of a bispecific Fc-containing antibody, variable regions, or fragments thereof, derived from antibodies with yet other binding specificities are appended to the N- or C-terminus of the IgG scaffold to yield a tetravalent, multi-specificity antibody-like molecule. Combinations of variable regions, or their fragments, of antibodies with diverse binding specificities, which may be incorporated into scFvs, can be appended to an IgG scaffold or other scaffold to yield multi-valent, multi-specificity antibody-like molecules. Antibodies of the invention can be incorporated into mini-antibodies wherein scFvs are fused to the C-terminus of the CH3 portion or hinge region of an antibody Fc region. Divalent (or bivalent) single-chain variable fragments (di-scFvs, bi-scFvs) can be engineered by linking two scFvs. This can be done by producing a single peptide chain with two V_{H} and two Y_{L} regions, yielding tandem scFvs. Another possibility is the creation of scFvs with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Consequently, diabody drugs could be dosed much lower than other therapeutic antibodies and are capable of highly specific targeting of tumors in vivo. Still shorter linkers (one or two amino acids), and optional addition of disulfide bonds, lead to the formation of trimers, so-called triabodies or tribodies. Tetrabodies and higher order multimers have also been produced They exhibit an even higher affinity to their targets than diabodies.

Other formats of a bispecific antibody-like molecules are bispecific tandem di-scFvs and bispecific T cell engagers, known as BiTEs (BiTE antibody constructs). BiTEs are fusion proteins consisting of two scFvs of different antibodies, on a single peptide chain of about 55 kilodaltons. Typically, one of the binding arms binds to a molecule on a T cell, such as the CD3 receptor, and the other binding arm binds to a tumor cell via a tumor specific molecule, such as aberrantly expressed MUC1*, All of these formats can be composed from one or more variable fragments of antibodies of the invention, with specificity for at least two different antigens, to generate multi-valent and multi-specific antibody-like molecules. In one aspect, a variable domain fragment derived from a first antibody binds to a first antigen on a first surface and a variable domain fragment derived from a second antibody binds to a second antigen on a second surface, wherein at least one of the surfaces may be the surface of a cell. In another aspect of the invention, at least one of the variable domain fragments comprising a multi-valent, multi-specific antibody-like molecule binds to an antigen that is not associated with a surface. In one aspect, the antigen that is not associated with a surface is a cytokine.

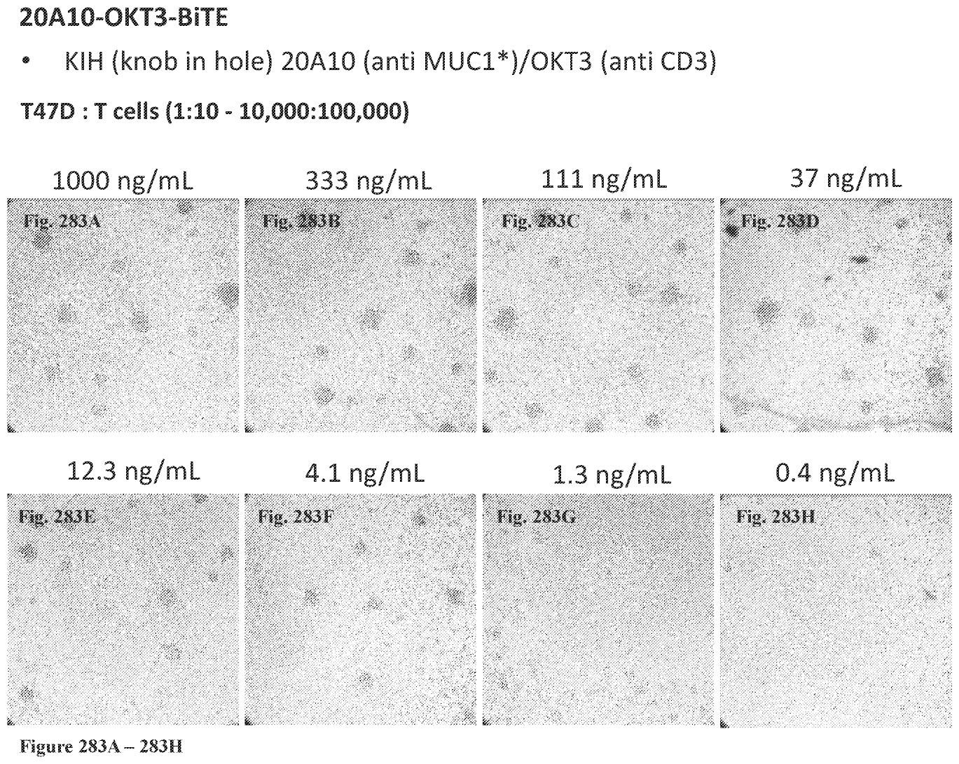

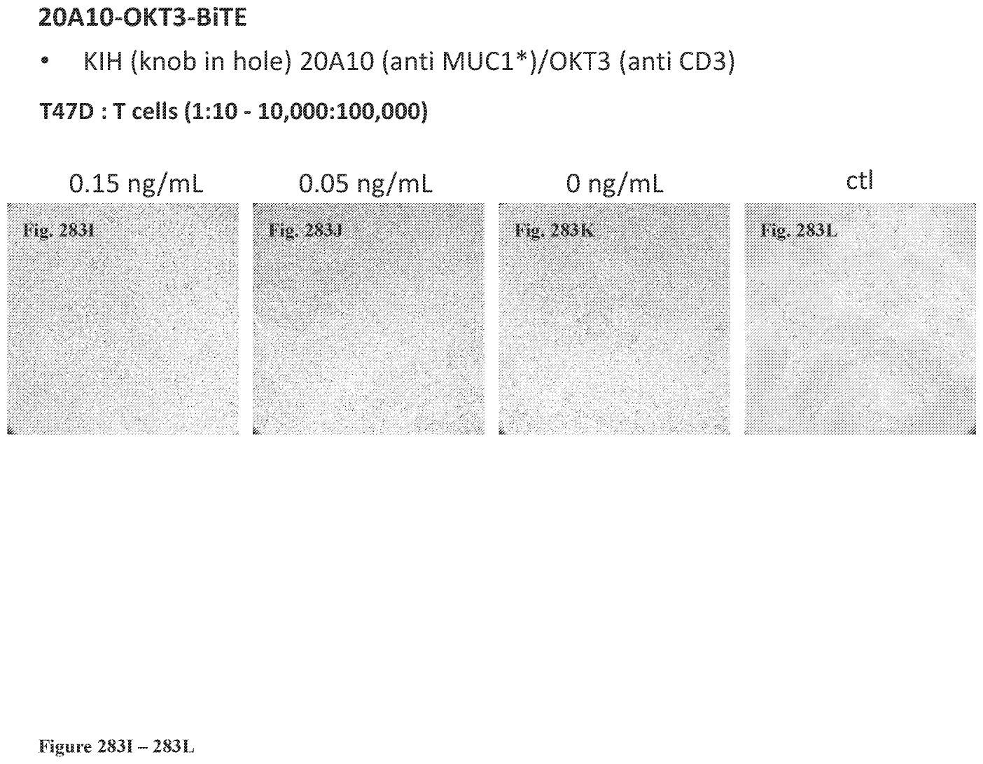

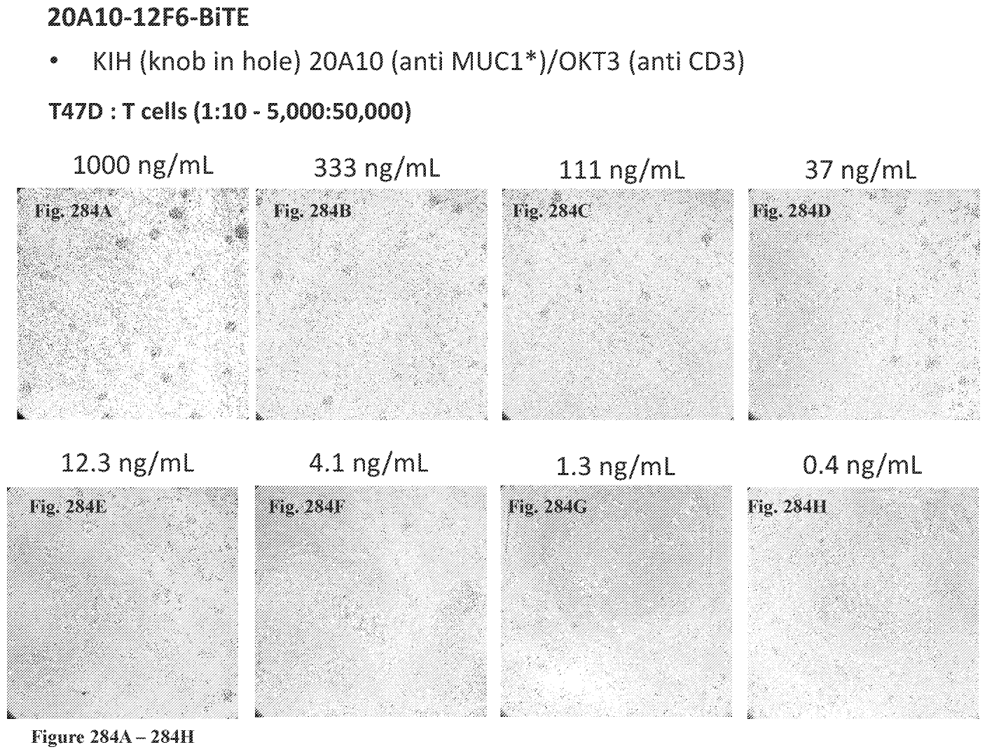

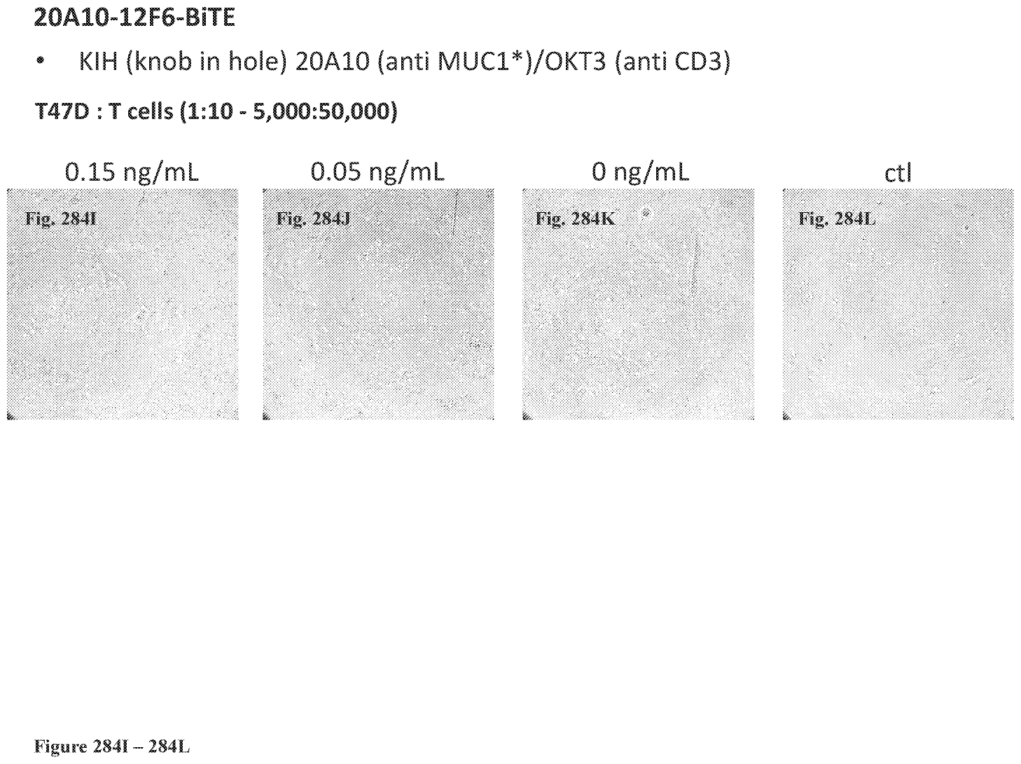

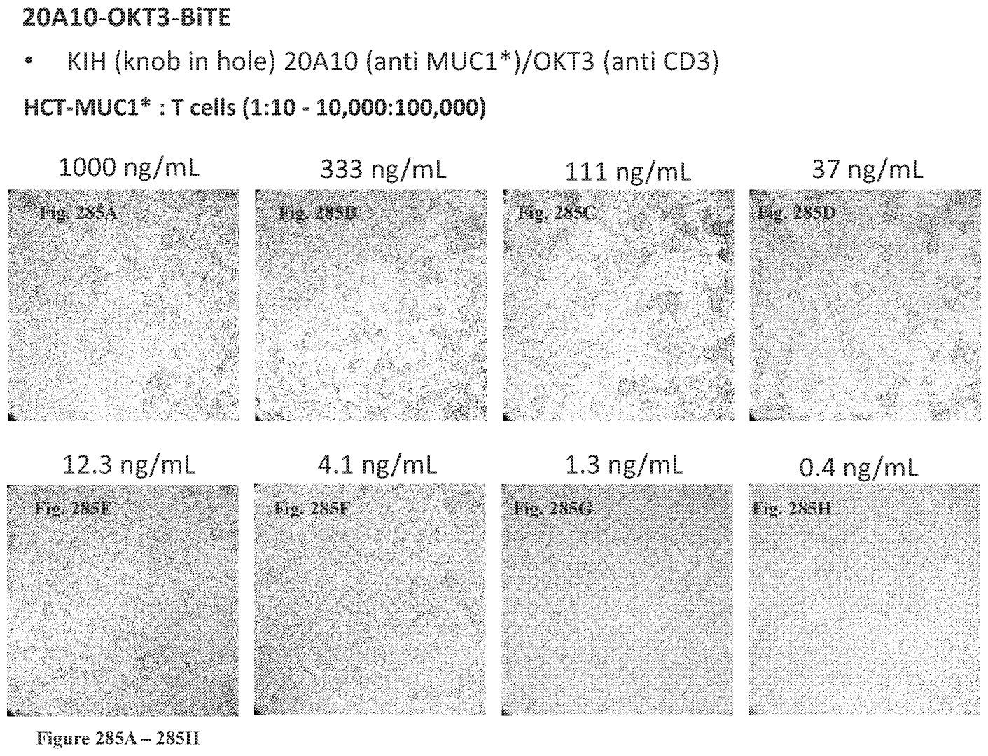

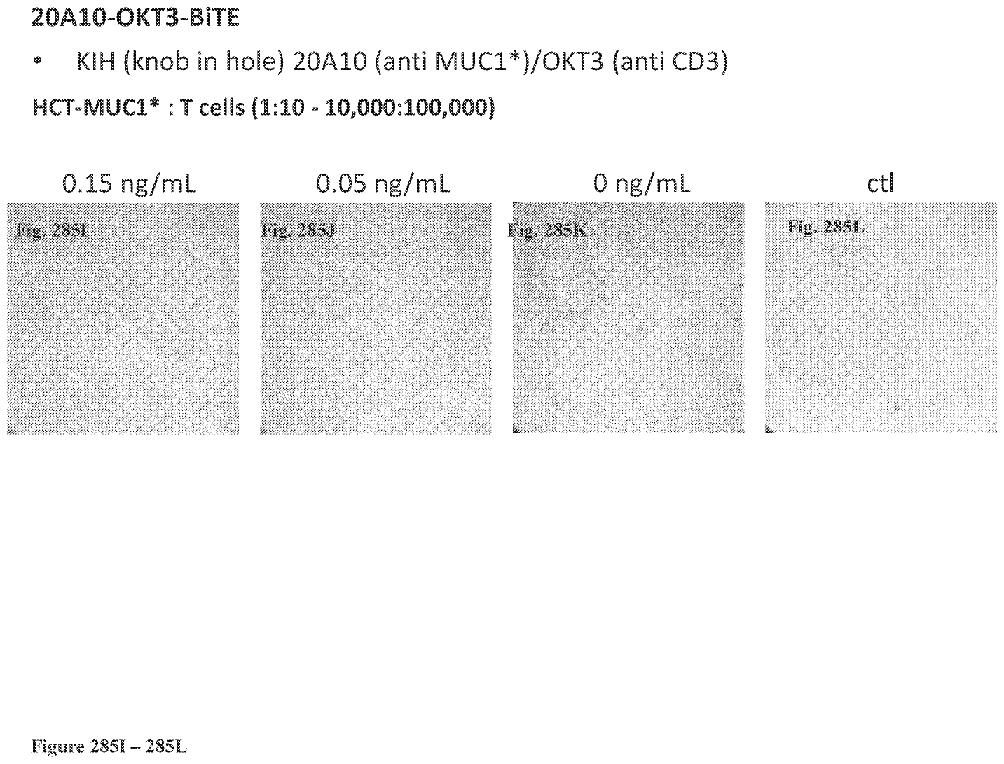

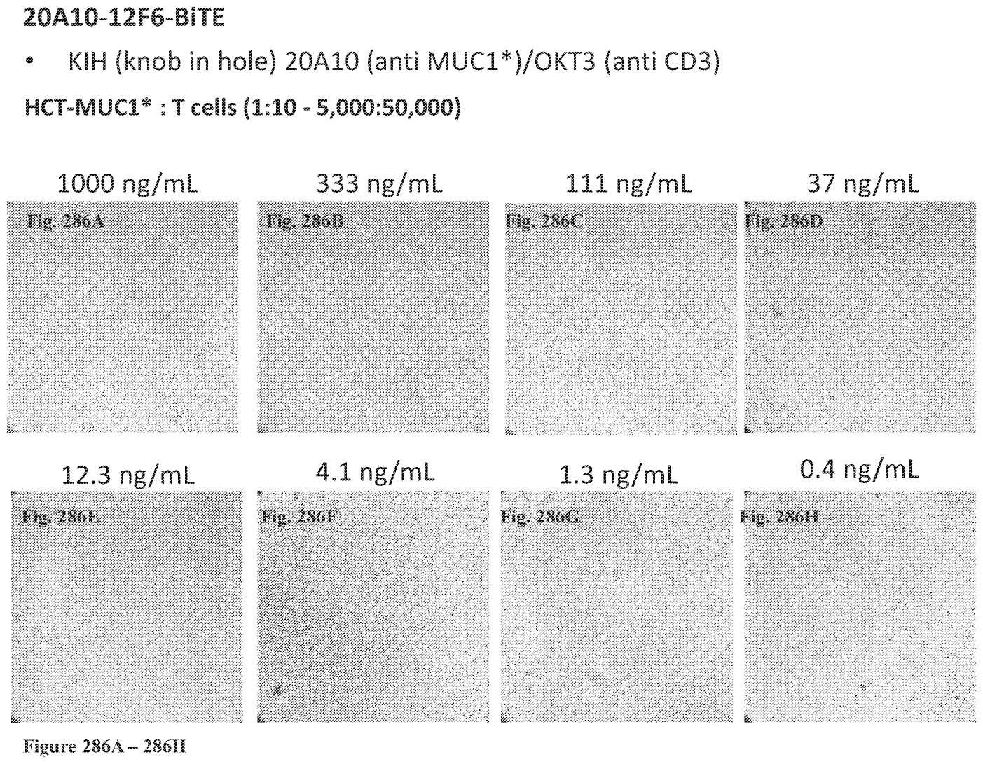

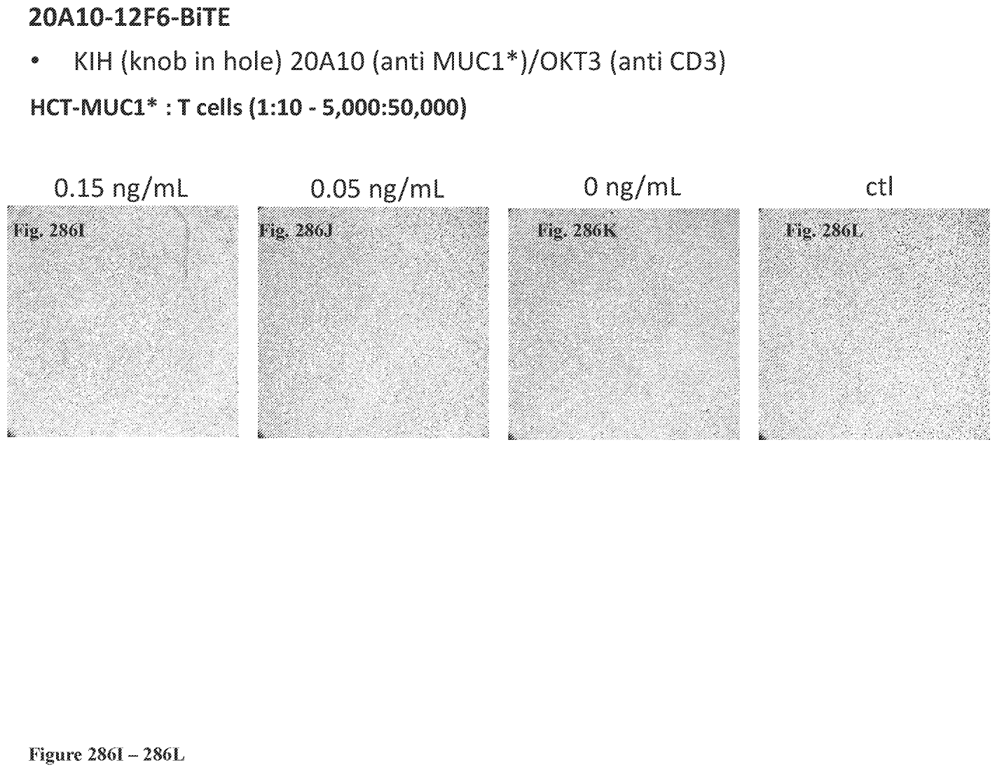

As an example of how antibodies of the invention can be incorporated into bispecific antibodies, we constructed a bispecific antibody using a knob-in-hole, also known as KTH (Spiess et al. Molecular Immunology 67, 95-106 (2015)), format. In this example, a first arm of the antibody is the humanized anti-MUC1* antibody 20A10, also known as hu20A10, with a 14616 framework region; the second arm of the antibody is either the anti-CD3 antibody OKT3 or 12F6, which both bind to the same epitope on human T cells. The resultant bispecific antibodies are referred to here as 20A10-OKT3-BiTE and 20A10-12F6-BiTE. In a demonstration of function, the bispecific antibodies are added at various concentrations to cells in culture wherein both human T cells and MUC1* positive cancer cells are present. In one case the cancer cells are T47D breast cancer cells and in the other case a MUC1* negative line HCT-116 colon cancer cells have been transduced to express MUC1*, called HCT-MUC1*. As can be seen in the photographs shown in FIGS. 283A-283L, FIGS. 284A-284L, FIGS. 285A-285L and FIGS. 286A-286L, the addition of either bispecific antibody mediated the joining together of the T cells and the MUC1* positive cancer cells as evidenced by a bispecific dose-dependent cell clustering. Two control experiments were performed. In one control, no bispecific antibody is added, but both T cells and MUC1* cancer cells are present. No clustering is observed. In another control, bispecific antibody is added to MUC1* positive cancer cells, but no T cells are present. These data demonstrate that anti-MUC1* antibodies of the invention can be readily incorporated into a bispecific format. In these examples, the second arm of the bispecific antibody was an antibody that recognizes CD3 on T cells. However, it is not intended that the invention be limited to bispecific antibodies where one arm binds to MUC1* and the other binds a T cell. A person skilled in the art could readily substitute the second arm of the bispecific antibody with an antibody that binds to art NK cell, or another receptor on a cancer cell for increased affinity. As MNC2 binds to the same epitope as 20A10, it is evident that a person skilled in the art could readily substitute MNC2 or humanized MNC2 for the 20A10. In one aspect of the invention a person diagnosed with a MUC1* positive cancer, or at risk of developing a MUC1* positive cancer, is treated with art effective amount of a bispecific antibody that simultaneously binds to a. MUC1* positive cancer cell and a human T cell. In another aspect of the invention, the bispecific antibody is hu20A10-OKT3-BiTE. In another aspect of the invention, the bispecific antibody is hu20A10-12F6-BiTE. In another aspect of the invention, the bispecific antibody is huMNC2-OKT3-BiTE. In another aspect of the invention, the bispecific antibody is huMNC2-12F6-BiTE.

Another aspect of the invention is a method for treating a patient diagnosed with, suspected of having, or at risk of developing a MUC1 positive or MUC1* positive cancer, wherein the patient is administered an effective amount of one of the multi-valent, multi-specific antibody-like molecules described here wherein at least one of the variable domains, or fragment thereof, is derived from an antibody that binds to PSMGFR (SEQ ID NO:2), or more specifically to N-10 peptide (SEQ ID NO:3), or more specifically to N-19 peptide (SEQ ID NO:4), or N-23 peptide (SEQ ID NO:5), or N-26 peptide (SEQ ID NO:6), or N-30 peptide (SEQ ID NO:7) or still more specifically that binds to a fragment of the PSMGFR peptide wherein said binding depends on the presence of the amino acids FPFSAQSGA (SEQ ID NO:10). In one case, the antibody variable fragment that binds to MUC1* comprises portions of MNC2, MNE6, 20A10, 3C2B1, 5C6F3, 25E6, 18G12, 28F9, 1E4, B12, B2, B7, B9, 8C7F3, or H11. In one case, at least one of the antibody variable fragments comprising a multi-valent, multi-specific antibody-like molecule that binds to MUC1* comprises portions of MNC2, MNE6, 20A10, 3C2B1, 5C6F3, 25E6, 18G12, 28F9, 1E4, B12, B2, B7, B9, 8C7F3, or H11 and at least one other antibody variable fragment binds to an antigen on an immune cell, which may be a T cell or NK cell.

Another aspect of the invention is a method for treating a patient diagnosed with, suspected of having, or at risk of developing a MUC1 positive or MUC1* positive cancer, wherein the patient is administered an effective amount of a BiTE wherein one antibody variable fragment of the BiTE binds to a T cell surface antigen and the other antibody variable fragment of the BiTE binds to PSMGFR (SEQ ID NO:2), or more specifically to N-10 peptide (SEQ ID NO:3), or more specifically to N-19 peptide (SEQ ID NO:4), or N-23 peptide (SEQ ID NO:5), or N-26 peptide (SEQ ID NO:6), or N-30 peptide (SEQ ID NO:7) or still more specifically that binds to a fragment of the PSMGFR peptide wherein said binding depends on the presence of the amino acids FPFSAQSGA (SEQ ID NO:10). In one case, the antibody variable fragment of the BiTE that binds to MUC1* comprises portions of MNC2, MNE6, 20A10, 3C2B1, 5C6F3, 25E6, 18G12, 28F9, 1E4, B12, B2, B7, B9, 8C7F3, or H11.

In another aspect of the invention, MUC1* peptides including PSMGFR (SEQ ID NO:2), or most or all of N-10 peptide are used in adoptive T cell approaches. In this case, a patient's T cells are exposed to the MUC1* peptides and through various rounds of maturation, the T cells develop MUC1* specific receptors. The adapted T cells are then expanded and administered to the donor patient who is diagnosed with, suspected of having, or is at risk of developing a MUC1* positive cancer.

A series of CARs were also made that had MNC2 and humanized MNC2 as the extra cellular, targeting head of the CAR. The constructs for these CARs were inserted into a plasmid that was then inserted into a Lenti viral vector. Human T cells were then transduced with the lenti viral vector carrying the MNC2 CARs and huMNC2 CARs. MNC2-scFv-CARs that were mouse sequence or humanized were generated. In one aspect of the invention, the CAR comprised huMNC2-scFv-short hinge region-transmembrane domain derived from CD8-short intracellular piece-4-1BB-3zeta. In another aspect, the transmembrane domain was derived from CD4 transmembrane sequence. In another aspect, the intracellular co-stimulatory domain was CD28-3zeta. In yet another aspect, the intracellular co-stimulatory domain was CD28-4-1BB-3zeta.

There are a variety of methods for assessing whether or not T cells recognize a target cell and are in the process of mounting an immune response. T cells cluster when they recognize a target or foreign cell. This can be readily seen with the naked eye or at low magnification. The appearance of CAR T cell clustering when co-cultured with target cancer cells is one measure of: a) whether or not they recognize the cells as target cells; and b) whether or not they are getting activated to attack the targeted cells, which in this case are cancer cells. FIGS. 45-47 show photographs of MUC1* positive T47D breast cancer cells that were either stably transfected with mCherry or dyed with CMTMR, so are red, which were co-cultured with either human T cells without a CAR or human T cells transduced with huMNC2-scFv-CAR44, or with huMNC2-scFv-CAR50. The CAR T cells are clear. As can be seen, there is no T cell induced clustering of the cancer cells when the T cell does not carry a CAR. However, when T cells carrying a MUC1* targeting CAR, there is dramatic clustering of the MUC1* positive cancer cells.

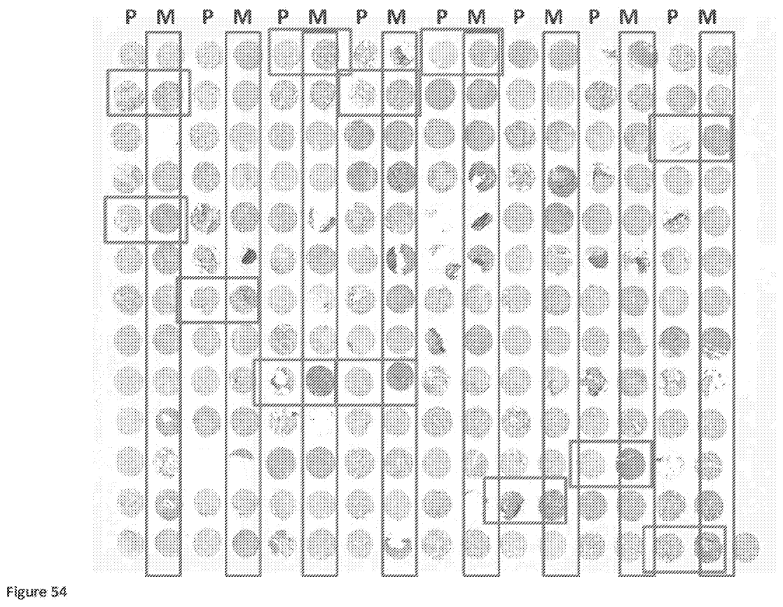

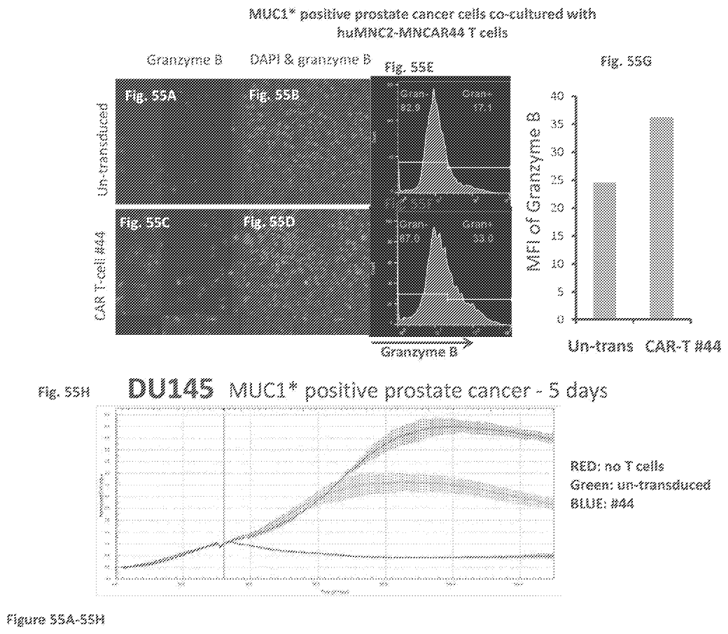

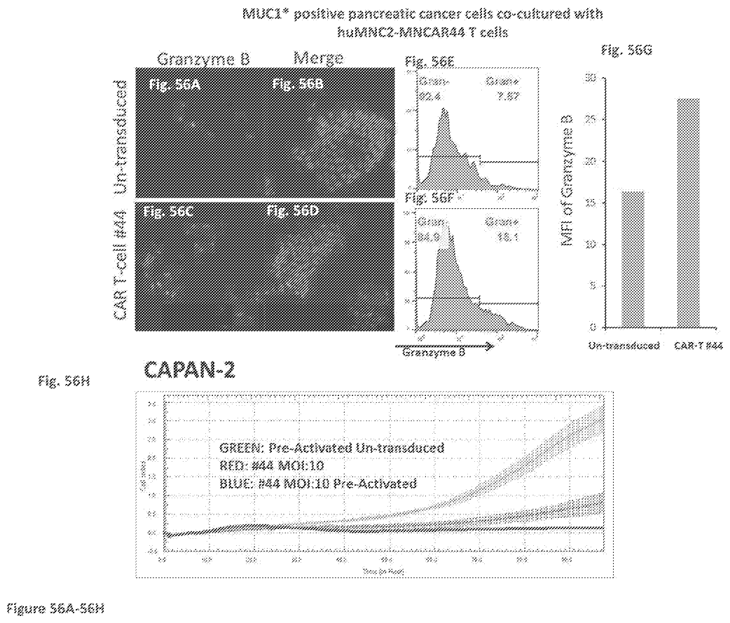

After T cells recognize and cluster target cells, they overexpress perform and granzyme B. Together these two molecules activate a cell death pathway in the targeted cell. It is thought that the perform makes a hole in the target cell into which the T cell injects granzyme B which then activates apoptotic proteases, causing the target cell to lyse. FIG. 55 and FIG. 56 show huMNC2-scFV-CAR44 T cells binding to target MUC1* positive prostate cancer and pancreatic cancer cells and injecting granzyme B.

Another measure of whether or not a T cell has recognized a target cell and is activated to kill that cell, is the upregulation and secretion of cytokines, interferon gamma (IFN-g) and interleukin-2 (IL-2), by the T cell. Activation of CAR T cells, as evidenced by IFN-g and IL-2 secretion, can be readily measured in vitro. CAR T cells are co-cultured with target cells and after an incubation period, the conditioned media is assayed by ELISA to detect secreted IFN-g and IL-2. In order to determine the cancer-specificity of CAR T cells wherein the targeting head of the CAR was either huMNC2 or huMNE6, these experiments were performed with huMNC2-CAR44 T cells and huMNE6-CAR44 T cells in co-culture with MUC1* positive cancer cells and normal cells. Table 1 details the MUC1 positive normal or primary cells that were tested.

TABLE 1
Normal Cell Lines and Primary Cells

| | ATCC | | |
|---|---|---|---|
| Cell Line | Designation | Tissue | Origin |
| Hep.G2 | | Liver | |
| THLE-3 | CRL-11233 | Liver | The THLE-2 (ATCC CRL-10149 and the |
| | | | THLE-3 (ATCC CRL-11233) cell lines |
| | | | were derived from primary normal liver |
| | | | cells by infection with SV40 large T |
| | | | antigen. THLE-2 and THLE-3 cells |
| | | | express phenotypic characteristics of |
| | | | normal adult liver epithelial cells. They are |
| | | | nontumorigenic when injected into |
| | | | athymic nude mice, have near-diploid |
| | | | karyotypes, and do not express alpha- |
| | | | fetoprotein. |
| Lonza | HUM181141 | Liver | Male, Caucasian |
| Primary | | | 2.0 months old |
| Hepatocytes | | | Induction Fold CYP1A2 (a) 14.0 |
| | | | Induction Fold CYP2B6 (b) 13.0 |
| | | | Induction Fold CYP3A4 (c) 44.0 |
| | | | Basal Activity CYP1A2 2.6 |
| | | | Basal Activity CYP2B6 0.7 |
| | | | Basal Activity CYP3A4 14.0 |
| | | | Additional Information: |
| | | | Inducer/Marker Metabolite |
| | | | (a) 0.05 mM Omeprazole/Acetaminophen |
| | | | (b) 1 mM Phenobarbital/ |
| | | | Hydroxybupropion |
| | | | (c) 0.01 mM Rifampicin/6-Beta- |
| | | | Hydroxytestosterone |
| | | | Basal activity is expressed as: |
| | | | pmol/million cells/minute |
| T/G HA- | CRL-1999 | Aortic Smooth | 11 months |
| VSMC | | Muscle | Female, Caucasian |
| CCD-18Lu | CCL-205 | Lung | This fibroblast-like cell line was derived |
| | | | from the lung tissue of a 2 month, 17-day- |
| | | | old Black female. |
| | | | The donor had cerebral anoxia, cardiac |
| | | | anomaly, sepsis, endocardial cushion |
| | | | defect and fetal alcoholic syndrome. |
| | | | Female, Black |
| | | | 2.5 months |
| HBEC-5i | CRL-3245 | Brain | Derived from small fragments of human |
| | | endothelium | cerebral cortex obtained from patients who |
| | | | had died of various causes. |
| Hs | CRL-7869 | Stomach/Intestine | 18 weeks gestation fetus |
| 738.St/Int | | | Male, Caucasian |
| | | | Part of the NBL Cell Line Collection. This |
| | | | cell line is neither produced nor fully |
| | | | characterized by ATCC. We do not |
| | | | guarantee that it will maintain a specific |
| | | | morphology, purity, or any other property |
| | | | upon passage. |
| MCF-12A | CRL-10782 | Breast | The MCF-12A cell line is a non- |
| | | | tumorigenic epithelial cell line established |
| | | | from tissue taken at reduction |
| | | | mammoplasty from a nulliparous patient |
| | | | with fibrocystic breast disease that |
| | | | contained focal areas of intraductal |
| | | | hyperplasia. The line was produced by |
| | | | long term culture in serum free medium |
| | | | with low Ca++ concentration. MCF-12A |
| | | | was derived from adherent cells in the |
| | | | population. |
| Hs 1.Tes | CRL-7002 | Testis | Male, Caucasian |
| | | | second trimester |
| | | | Part of the NBL Cell Line Collection. This |
| | | | cell line is neither produced nor fully |
| | | | characterized by ATCC. We do not |
| | | | guarantee that it will maintain a specific |
| | | | morphology, purity, or any other property |
| | | | upon passage. |
| HRCE | Lonza: | Kidney | Human Renal Cortical Cells (HRCE) are |
| | catalogue # | | from proximal and distal tubules. |
| | CC-2554 Lot | | Donor info: 49 year old female, passage 2, |
| | # | | 95% viability, doubling time (hours) 24 hrs |
| | 0000542104 | | |

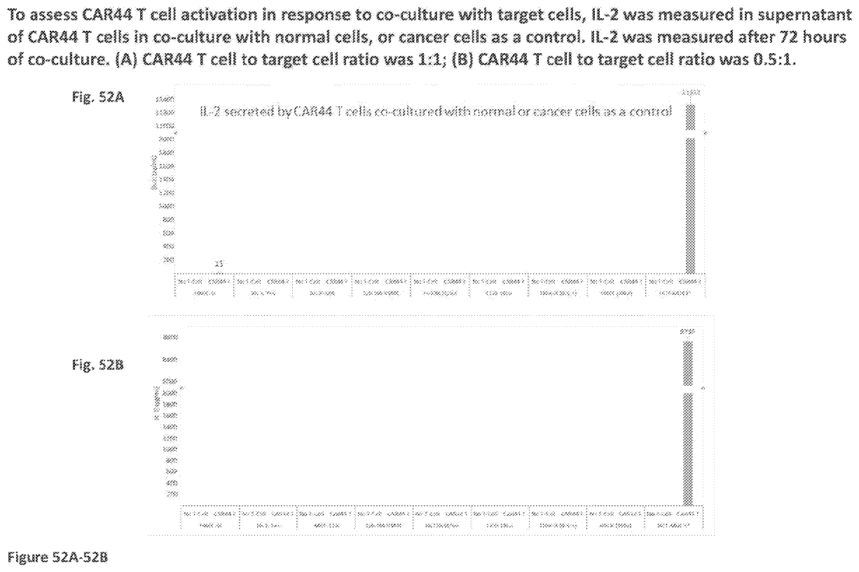

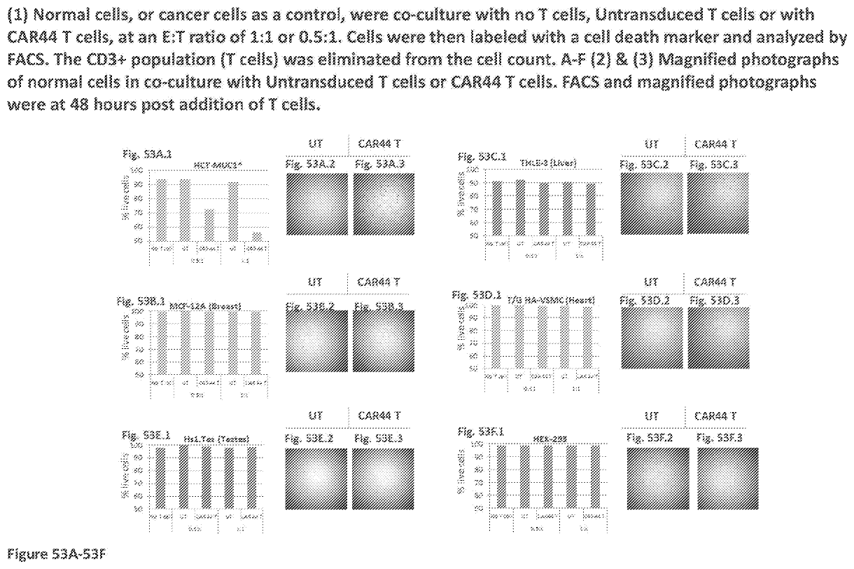

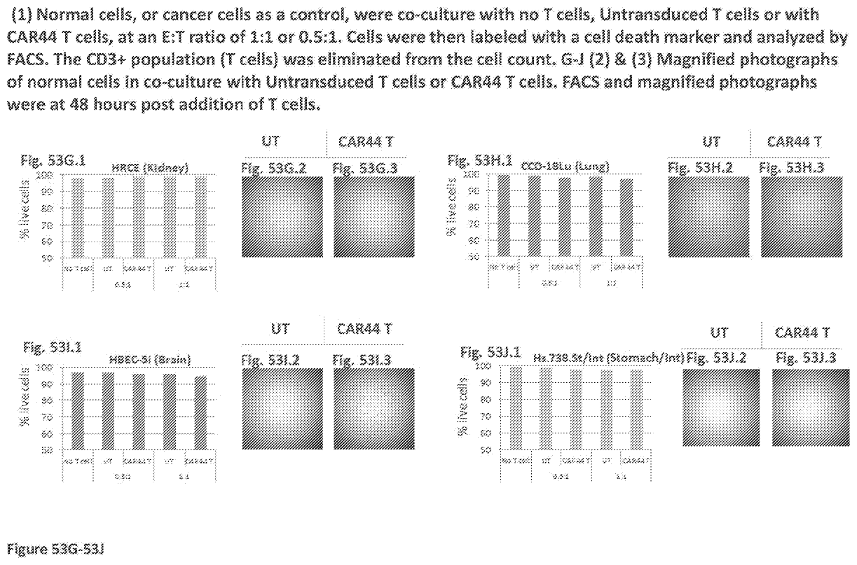

FIG. 50 is a graph of PCR measurement of the various cell lines tested, wherein mRNA levels of MUC1 are measured. The cancer cell lines that were tested in these assays were HCT-MUC1* and T47D breast cancer cells. These cells were co-cultured with huMNC2-CAR44 human T cells. Co-culture of huMNC2-CAR44 T cells with the cancer cells induced the CAR T cells to secrete large amounts of IFN-g and IL-2 into the surrounding media, yet co-culture with the MUC1 positive normal cells induced no secretion of the cytokines (FIG. 51 and FIG. 52). In addition to testing for IFN-g and IL-2 secretion by the CAR T cells, the normal cells were assayed for signs of cell death, which could have been induced by the CAR T cells if the antibody targeting head were not extremely cancer-specific. After co-culture with huMNC2-CAR44 T cells, the cells were incubated with a cell death marker, then assayed by FACS. huMNC2-CAR44 T cells induced no cell death in the normal cells (FIG. 53A-53J).

In addition to FACS analysis, many researchers now use an xCELLigence instrument to measure CAR T killing of cancer cells. FACS is not the best method for tracking T cell induced cell killing because the T cells lyse the target cell. By FACS it is difficult to measure dead cells because they are excluded as cell debris, so one must infer an amount of cell killing and by various methods determine if the missing cells are T cells or cancer cells.

The xCELLigence instrument uses electrode arrays upon which cancer cells are plated. The adherent cancer cells insulate the electrode and so cause an increase in impedance as they grow. Conversely, T cells are not adherent and remain in suspension so do not contribute to insulation of the electrode which would increase impedance. However, if the T cells or CAR T cells kill the cancer cells on the electrode plate, the cancer cells ball up and float off as they die, which causes the impedance to decrease. The xCELLigence instrument measures impedance as a function of time, which is correlated to cancer cell killing. In addition, the electrode plates also have a viewing window. When CAR T cells effectively kill the adsorbed target cancer cells, there is a decrease in impedance but also one can see that there are no cancer cells left on the plate surface.

FIGS. 55A-55H show the cytotoxic effect of huMNC2-CAR44 T cells on MUC1* positive DU145 prostate cancer cells as measured by a variety of assays. FIG. 55A is a fluorescent photograph of untransduced T cells co-cultured with the prostate cancer cells, wherein granzyme B is stained with a red fluorophore. FIG. 55C is a fluorescent photograph of huMNC2-CAR44 T cells co-cultured with the prostate cancer cells, wherein granzyme B is stained with a red fluorophore. FIG. 55D is the DAPI and granzyme B merge. FIG. 55E is a FACS scan for fluorescently labeled granzyme B for untransduced T cells incubated with the cancer cells. FIG. 55F is a FACS scan showing a positive increase in fluorescently labeled granzyme B for huMNC2-CAR44 T cells incubated with the cancer cells. FIG. 55G is a graph of the mean fluorescent intensity. FIG. 55H is an xCELLigence scan tracking the real-time killing of DU145 cancer cells by huMNC2-CAR44 T cells (blue trace) but not by untransduced T cells (green). FIGS. 56A-56H show the cytotoxic effect of huMNC2-CAR44 T cells on MUC1* positive CAPAN-2 pancreatic cancer cells as measured by a variety of assays. FIG. 56A is a fluorescent photograph of untransduced T cells co-cultured with the pancreatic cancer cells, wherein granzyme B is stained with a red fluorophore. FIG. 56B is the DAPI and granzyme B merge. FIG. 56C is a fluorescent photograph of huMNC2-CAR44 T cells co-cultured with the pancreatic cancer cells, wherein granzyme B is stained with a red fluorophore. FIG. 56D is the DAPI and granzyme B merge. FIG. 56E is a FACS scan for fluorescently labeled granzyme B for untransduced T cells incubated with the cancer cells. FIG. 56F is a FACS scan showing a positive increase in fluorescently labeled granzyme B for huMNC2-CAR44 T cells incubated with the cancer cells. FIG. 56G is a graph of the mean fluorescent intensity. FIG. 56H is an xCELLigence scan tracking the real-time killing of CAPAN-2 cancer cells by huMNC2-CAR44 T cells (blue trace) but not by untransduced T cells (green). FIGS. 57A-57C show xCELLigence scans tracking the real-time killing of MUC1* positive cancer cells, but not MUC1* negative cells, by huMNC2-CAR44 T cells. FIG. 57A shows that huMNC2-CAR44 T cells effectively kill HCT colon cancer cells that have been stably transfected with MUC1*. FIG. 57B shows that huMNC2-CAR44 T cells have almost no effect on HCT-MUC1-41TR, which is a MUC1 negative cancer cell that has been stably transfected with a MUC1 full-length. In this cell line only about 10% of the cell have MUC1 cleaved to MUC1*. FIG. 57C shows that huMNC2-CAR44 T cells have no effect on HCT-116 cells, which is a MUC1 negative colon cancer cell line.

These data demonstrate that T cells transduced with a CAR wherein the antibody fragment targeting head is MNC2, effectively kill MUC1* positive cancer cells. These data specifically show that huMNC2-scFV-CAR44 transduced into human T cells effectively kill MUC1* positive cancer cells. Because we and others have now demonstrated that the most important aspect of CAR T function is the targeting antibody fragment, it follows that an immune cell or a T cell transduced with any CAR having the antibody fragment MNC2-scFV or huMNC2-scFV would have similar efficacy against MUC1 or MUC1* positive tumors. For example, the hinge region that connects the scFv to the transmembrane portion could be any flexible linker. The intracellular co-stimulatory domains could be CD28-3zeta, CD28-4-1BB-3zeta or any combination of immune cell co-stimulatory domains.

FIG. 61 shows an experiment in which huMNC2-scFv-CAR44 transduced human T cell that were bead stimulated (Protocol 1) or cancer cell stimulated (Protocol 2) were tested for their ability to inhibit tumor growth in animals. Human cancer cells that had been stably transfected with Luciferase were injected into female NOD/SCID/GAMMA (NSG) mice between 11 and 15 weeks of age. 500,000 BT-20 breast cancer cells were injected sub-cutaneously into a rear flank. Tumor engraftment was verified by injecting the animals with Luciferin and then imaging the fluorescent cancer cells using an IVIS instrument. IVIS images taken Day 5 post implantation showed the presence of tumor cells. On Day 6 after IVIS measurement, animals were given a one-time injection of 10 million of either human T cells transduced with huMNC2-scFv-CAR44 or untransduced T cells. 5 million T cells were injected intra-tumor and 5 million were injected into the tail vein. 10 minutes prior to IVIS photographs, mice were IP injected with Luciferin, which fluoresces after cleavage by Luciferase, thus making tumor cells fluoresce. FIGS. 61A, 61D, 61G show photographs of mice that were treated with huMNC2-scFv-CAR44 T cells that had been pre-stimulated by co-culturing for 24 hours with 4 μm beads to which was attached a synthetic MUC1*, PSMGFR peptide 24 hours prior to administration, “Protocol 1”. FIGS. 61B, 61E, 61H show photographs of mice that were treated with huMNC2-scFv-CAR44 T cells that had been pre-stimulated by twice co-culturing for 24 hours with MUC1* positive cancer cells 24 hours prior to administration, “Protocol 2”. As can be seen in FIG. 61, huMNC2-CAR44 T cells that were peptide-bead stimulated inhibited tumor growth better than cells pre-stimulated by incubation with live cancer cells, which likely contaminated the target cells and increased the tumor volume.

huMNC2-scFv-CAR44 transduced human T cell that were bead stimulated (Protocol 1) or cancer cell stimulated (Protocol 2) were also tested for their ability to inhibit tumor growth in animals. Human cancer cells that had been stably transfected with Luciferase were injected into female NOD/SCID/GAMMA (NSG) mice between 11 and 15 weeks of age. In another experiment, 500,000 BT-20 MUC1* positive triple negative breast cancer cells were injected sub-cutaneously into a rear flank. Tumor engraftment was verified by injecting the animals with Luciferin and then imaging the fluorescent cancer cells using an IVIS instrument. IVIS images taken Day 6 post implantation showed the presence of tumor cells. On Day 6, after IVIS imaging, 10M huMNC2-scFv-CAR44 T cells were administered to the animals. 5M of the CAR T cells were administered by intratumor injection and the other 5M were administered by tail vein injection. Control group was injected by same administration routes with the same number of untransduced T cells. IVIS measurements of tumor burden were taken on Days 6, 8, and 12. As can be seen in FIGS. 61A-61J, both groups of mice treated with huMNC2-CAR44 T cells showed a decrease in tumor burden compared to the control group.

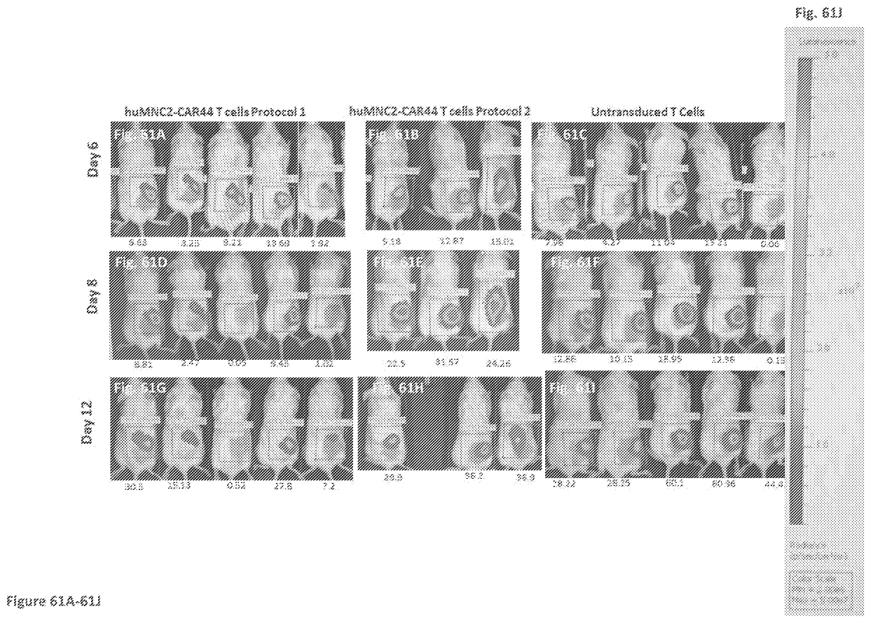

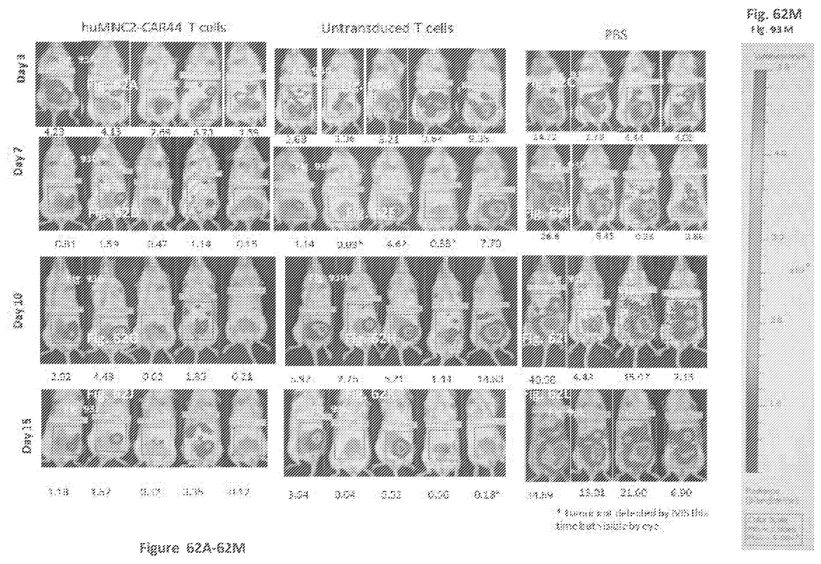

huMNC2-scFv-CAR44 transduced human T cell that were bead stimulated (Protocol 1) were also tested for their ability to inhibit ovarian cancer growth in animals. Human SKOV-3 MUC1* positive ovarian cancer cells that had been stably transfected with Luciferase were injected into female NOD/SCID/GAMMA (NSG) mice between 11 and 15 weeks of age. In one experiment, 500,000 SKOV-3 cancer cells were injected into the intraperitoneal cavity to mimic metastatic ovarian cancer in humans. Tumor engraftment was verified by injecting the animals with Luciferin and then imaging the fluorescent cancer cells using an IVIS instrument. IVIS images taken Day 3 post implantation showed the presence of tumor cells. On Day 4 and Day 11, post tumor implantation, 10M huMNC2-scFv-CAR44 T cells were IP administered to the animals. On Day 4, CAR T cells were IP injected. On Day 11 half the CAR T cells were injected into the intraperitoneal space and the other half was injected into the tail vein. Control groups were injected by same administration routes with either the same number of untransduced T cells or same volume of PBS. Subsequent IVIS measurements of tumor burden were taken on Day 7, Day 10 and Day 15. As can be seen in FIGS. 62A-62L, control mice have tumors that are growing at a much faster rate than the huMNC2-CAR44 T cell treated mice. FIG. 62M shows the IVIS color bar correlating photons/second to color.

One aspect of the invention is a method for treating a patient diagnosed with, suspected of having, or at risk of developing a MUC1 positive or MUC1* positive cancer, wherein the patient is administered an effective amount of an antibody, antibody fragment which may be incorporated into a BiTE, a bispecific antibody, a multi-specific antibody, an ADC or a CAR expressed in an immune cell wherein the antibody or fragment thereof is derived from an antibody of the invention. In a preferred embodiment, the antibody or fragment thereof is derived from MNC2, MNE6, 20A10, 3C2B1, 5C6F3 or 25E6.

One aspect of the invention is a method for treating a patient diagnosed with, suspected of having, or at risk of developing a MUC1 positive or MUC1* positive cancer, wherein the patient is administered an effective amount of immune cells that have been transduced with a MUC1* targeting CAR, wherein the CAR is chosen from among the group consisting of MNE6-CD8-CD28-3z (SEQ ID NOS:297-298); MNE6-CD4-CD28-3z (SEQ ID NOS:748-749); MNE6-CD8-41BB-3z (SEQ ID NOS:300-301); MNE6-CD4-41BB-3z (SEQ ID NOS:750-751); MNE6-CD8-CD28-41BB-3z (SEQ ID NOS:303-304); MNE6-CD4-CD28-41BB-3z (SEQ ID NOS:754-755); MNE6scFv-Fc-8-41BB-CD3z (SEQ ID NOS:310-311); MNE6scFv-IgD-Fc-8-41BB-CD3z (SEQ ID NOS:770-771); MNE6scFv-FcH-8-41BB-CD3z (SEQ ID NOS:315-316); MNE6scFv-IgD-FcH-8-41BB-CD3z (SEQ ID NOS:772-773); MNE6scFv-Fc-4-41BB-CD3z (SEQ ID NOS:318-319); MNE6scFv-FcH-4-41BB-CD3z (SEQ ID NOS:321-322); MNE6scFv-IgD-8-41BB-CD3z (SEQ ID NOS:323-324); MNE6scFv-IgD-4-41BB-CD3z (SEQ ID NOS:327-328); MNE6scFv-X4-8-41BB-CD3z (SEQ ID NOS:330-331); MNE6scFv-X4-4-41BB-CD3z (SEQ ID NOS:333-334); MNE6scFv-8-4-41BB-CD3z (SEQ ID NOS:336-337), or any of the aforementioned CARs wherein the MNE6 is replaced by fragment derived from MNC2, MNE6, 20A10, 3C2B1, 5C6F3, 25E6, 18G12, 28F9, 1E4, B12, B2, B7, B9, 8C7F3, or H11. Another aspect of the invention is a method for treating a patient diagnosed with, suspected of having, or at risk of developing a cancer, wherein the patient is administered an effective amount of immune cells that have been transduced with one of the aforementioned CARs wherein the MNE6 is replaced by a peptide comprising antibody variable domain fragments that are specific for a cancer antigen. In any of the above methods, the immune cell may be a T cell and may further be isolated from the patient to be treated. Alternatively, the immune cell, which may be a T cell is isolated from a donor. In yet another aspect, the immune cell is derived from a stem cell that has been directed to differentiate to that immune cell type in vitro. In another aspect, a CAR containing sequences of the antibody are expressed in a stem cell, which then may be differentiated into an immune cell. In one case, the immune cell is a T cell. In another case, the immune cell is an NK cell.

Another aspect of the invention is a method for treating a patient diagnosed with, suspected of having, or at risk of developing a MUC1 positive or MUC1* positive cancer, wherein the patient is administered an effective amount of immune cells that have been transduced with a MUC1* targeting CAR. In a preferred embodiment, the CAR may include a single chain antibody fragment, scFv, comprising a sequence derived from antibody MNC2, such as MNC2-scFv (SEQ ID NO:239, 241, 243, or 654-655), from antibody 20A10, such as SEQ ID NO:1574-1581 or 5001-5012, from antibody 3C2B1, such as SEQ ID NO:1572-1573, which may be humanized for example as in SEQ ID NO:1812-1813, or from antibody 5C6F3, such as SEQ ID NO:1384-1385), which may be humanized for example as in SEQ ID NO:1814-1815, or from antibody 25E6 such as SEQ ID NO:1598-1599 or 1600-1601, wherein the hinge and transmembrane sequences may be derived from CD8 (SEQ ID NO:346 and SEQ ID NO:364), or from CD28 (SEQ ID NO:350 and SEQ ID NO:368), further comprising a co-stimulatory domain, which may be 41BB (SEQ ID NO:659) or CD28 (SEQ ID NO:378) and the CD3-zeta signaling domain may be derived from (SEQ ID NO:661) or may contain mutations including those referred to as 1XX (SEQ ID NO:1796-1797).

### Other MUC1 Cleavage Sites

It is known that MUC1 is cleaved to the growth factor receptor form, MUC1*, on some healthy cells in addition to cancer cells. For example, MUC1 is cleaved to MUC1* on healthy stem and progenitor cells. A large percentage of bone marrow cells are MUC1* positive. Portions of the intestine are MUC1* positive.

The inventors have discovered that MUC1 can be cleaved at different positions that are relatively close to each other but the location of cleavage changes the fold of the remaining portion of the extracellular domain. As a result, monoclonal antibodies can be identified that bind to MUC1* cleaved at a first position but do not bind to MUC1* that has been cleaved at a second position. This discovery is disclosed in WO2014/028668, filed Aug. 14, 2013, the contents of which are incorporated by reference herein its entirety. We identified a set of anti-MUC1* monoclonal antibodies that bind to MUC1* as it appears on cancer cells but do not bind to MUC1* as it appears on stem and progenitor cells. Conversely, we identified a second set of monoclonal antibodies that bind to stem and progenitor cells but do not bind to cancer cells. One method used to identify stem specific antibodies is as follows: supernatants from monoclonal hybridomas were separately adsorbed onto 2 multi-well plates. Stem cells, which are non-adherent cells, were put into one plate and cancer cells which are adherent were put into an identical plate. After an incubation period, the plates were rinsed and inverted. If the non-adherent stem cells stuck to the plate, then the monoclonal antibody in that particular well recognizes stem cells and will not recognize cancer cells. Antibodies that did not capture stem cells or antibodies that captured cancer cells were identified as cancer specific antibodies. FACS analysis has confirmed this method works.

Antibodies MNE6 and MNC2 are examples of cancer-specific antibodies. Antibodies MNC3 and MNC8 are examples of stem-specific antibodies. Although both sets of antibodies are able to bind to a peptide having the PSMGFR sequence, FACS analysis shows that the anti-MUC1* polyclonal antibody and MNC3 bind to MUC1* positive bone marrow cells but MNE6 does not. The MUC1* polyclonal antibody was generated by immunizing a rabbit with the PSMGFR peptide. Similarly, MNC3 binds to stem cells of the intestinal crypts but MNE6 does not. Conversely, MNE6 antibody binds to cancerous tissue while the stem-specific MNC3 does not. Competition ELISA experiments indicate that the C-terminal 10 amino acids of the PSMGFR peptide are required for MNE6 and MNC2 binding, but not for MNC3 and MNC8. Therefore, another method for identifying antibodies that are cancer specific is to immunize with a peptide having the sequence of the PSMGFR peptide minus the 10 N-terminal amino acids or use that peptide to screen for antibodies or antibody fragments that will be cancer specific. Antibodies that bind to a peptide with a sequence of PSMGFR peptide minus the N-terminal 10 amino acids, referred to herein as N-10 peptide, but do not bind to a peptide with a sequence of PSMGFR peptide minus the C-terminal 10 amino acids, C-10 peptide, are cancer specific antibodies for use in the treatment or prevention of cancers.

The extracellular domain of MUC1 is also cleaved on stem cells and some progenitor cells, where activation of cleaved MUC1 by ligands NME1 in dimer form or NME7 promotes growth and pluripotency and inhibits differentiation. The transmembrane portion of MUC1 that remains after cleavage is called MUC1* and the extracellular domain is comprised essentially of the Primary Sequence of MUC1 Growth Factor Receptor (PSMGFR) sequence. However, the exact site of cleavage can vary depending on cell type, tissue type, or which cleavage enzyme a particular person expresses or overexpresses. In addition to the cleavage site that we previously identified which leaves the transmembrane portion of MUC1* comprising most or all of the PSMGFR (SEQ ID NO:2), other cleavage sites could possibly result in an extended MUC1* comprised of most or all of SNIKFRPGSVVVQLTLAFREGTINVHDVETQFNQYKTEAASRY (SEQ ID NO:620); or

SVVVQLTLAFREGTINVHDVETQFNQYKTEAASRY (SEQ ID NO:621).

To test this hypothesis, and to determine if antibodies to an N-terminally extended PSMGFR, would generated more cancer-specific antibodies than antibodies that bind to the PSMGFR, we generated monoclonal antibodies by immunization with peptides:

(PSMGFR)
(SEQ ID NO: 2)
GTINVHDVETQFNQYKTEAASRYNLTISDVSVSDVPFPFSAQSGA,
(N + 20/C − 27)
(SEQ ID NO: 822)
SNIKFRPGSVVVQLTLAFREGTINVHDVETQFNQYKTE,
or
(N + 9/C − 9)
(SEQ ID NO: 824)
VQLTLAFREGTINVHDVETQFNQYKTEAASRYNLTISDVSVSDVP

Monoclonal antibodies generated from immunization with the same peptide can also show differences in reactivity to the same cancerous tissue specimen. These results indicate that the monoclonal antibodies recognize different conformations of the truncated MUC1 extra cellular domain produced by immunizing with different length peptides, mimicking different cleavage sites, or from cleavage at different sites in the host animal. Antibodies that recognize different cleavage site conformations may be cancer sub-type specific or patient specific, depending on which cleavage enzyme their tumor expresses. In one aspect of the invention, a patient diagnosed with a certain type of cancer is treated with an antibody of the invention that recognizes a cleaved MUC1 wherein the antibody is specific for cleavage by a specific enzyme that is known to be typically expressed by that sub-type of cancer. In another aspect, a patient tumor is analyzed to determine which enzyme his or her tumor expresses and an antibody that recognizes a MUC1 cleaved by that enzyme is then administered to the patient for the treatment of their cancer. The antibody may be in the form of a CAR, a BiTE, an ADC, a bispecific antibody, with or without an FC region or a portion of an Fc region, a bi-scFv, a di-scFv, a tandem di-scFv, a diabody, triabody, tribody, tetrabody and other antibody-like molecules that are multi-valent and multi-specific.

We previously reported that it is the MUC1 transmembrane cleavage product, called MUC1* (muk1 star), that mediates tumor growth and not full-length MUC1 (Mahanta et al 2008). MUC1* is a growth factor receptor that is activated by ligand induced dimerization of its short extra cellular domain (FIG. 1A). Dimerization of the MUC1* extra cellular domain activates the MAP kinase signaling cascade and stimulates growth and survival of cancer cells (Fessler et al 2009). Bivalent antibodies that dimerize the MUC1* extra cellular domain stimulate cancer cell growth while the monovalent Fab of the same antibody, which cannot dimerize, inhibits cancer cell growth. We demonstrated this in vitro (FIG. 1B) and in vivo (FIG. 7A-7B).

We then identified the natural ligands that dimerize and activate MUC1* growth factor receptor function. Dimers of NME1 bind to and dimerize the MUC1* extra cellular domain and stimulate growth (FIG. 1C and Smagghe et al 2013). NME1 can turn its growth factor properties off NME1 is secreted by MUC1* positive cells. Dimeric NME1 binds to MUC1* to stimulate growth. However, as the cell population grows, more and more NME1 is secreted from the cells. At high concentrations, the NME1 dimers multimerize and form hexamers, which do not bind to MUC1*, but likely bind to some unknown receptor, as the addition of NME1 hexamers turns off growth. NME1 is an adult form. The embryonic form is NME7_{AB} (Carter et al 2016). Each NME7A monomer has two binding sites for MUC1* so as a monomer it dimerizes MUC1* (FIG. 1D), stimulates growth and cannot turn itself off. In the developing embryo, BRD4 turns off NME7 and its co-factor JMJD6 turns on the self-regulating form, NME1. However, in cancers, NME7, which should be silenced in adult life, is aberrantly expressed again, where is renders the MUC1* growth factor receptor constitutively active.

In vitro, NME1 (SEQ ID NO:4) and NME7A (SEQ ID NO:827) bind to the PSMGFR portion of the MUC1* extra cellular domain. Both growth factors can bind to the PSMGFR peptide (SEQ ID NO:2) even if the 10 N-terminal amino acids are deleted, referred to herein as N-10 (SEQ ID NO:3). However, neither NME1 nor NME7A can bind to the PSMGFR peptide if the 10 membrane proximal amino acids are deleted (FIG. 2A-2D), referred to herein as C-10 (SEQ ID NO:825). In summary, the epitope to which NME1 and NME7A bind includes all or part of the 10 membrane proximal amino acids: PFPFSAQSGA (SEQ ID NO:1743). We tested various antibodies that were generated in animals by immunizing with the PSMGFR peptide for their ability to recognize cancer cells but not healthy cells. Among the most cancer selective were the MNC2 and MNE6 monoclonal anti-MUC1* antibodies. Two other monoclonal antibodies that were generated from immunizing animals with the PSMGFR peptide are MNC3 and MNC8. Although MNC2, MNE6, MNC3 and MNC8 all bind to the PSMGFR peptide, like NME1 and NME7_{AB}, MNC2 and MNE6 bind strongly to the N-10 peptide but not to the C-10 peptide (FIG. 2B-2C). In fact, MNC2 and MNE6 competitively inhibit the binding of NME1 and NME7_{AB} to PSMGFR (FIG. 3A-3C). Conversely, MNC3 and MNC8 bind to the C-10 peptide, bind less well to the N-10 peptide and do not compete with NME1 nor NME7A for binding to MUC1* peptides, including PSMGFR (FIG. 2E-2F). MNC3 and MNC8 are far less cancer specific than MNC2 and MNE6. MNC3 and MNC8 recognize stem and progenitor cells, such as hematopoietic stem cells, whereas MNC2 and MNE6 do not (FIG. 39-41). Because hematopoietic stem cells are the progenitor cells for the blood cells, it would be problematic to have a cancer therapeutic that would also target such an important normal cell type.

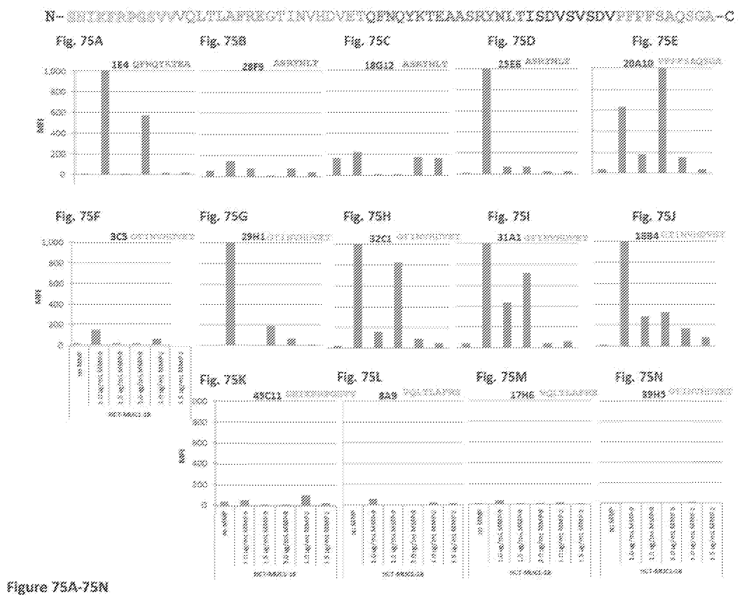

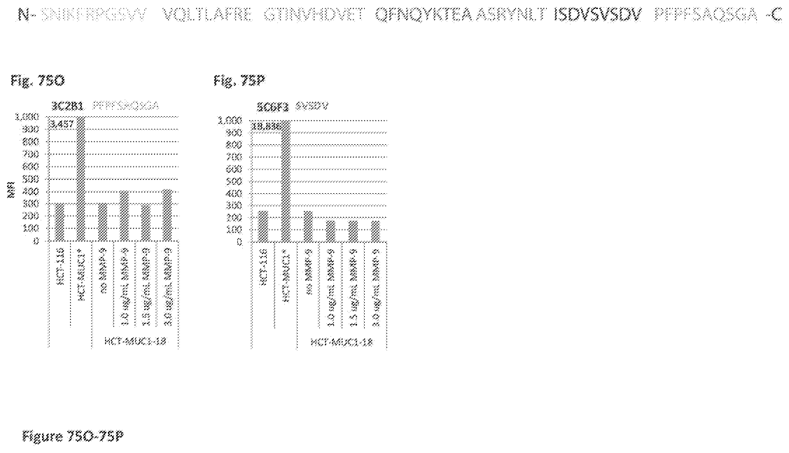

Because MUC1* is generated by enzymatic cleavage of MUC1, we researched which cleavage enzymes cleave MUC1 to a MUC1* and whether or not we could identify antibodies that would recognize a MUC1* generated by a first cleavage enzyme but not MUC1* generated by a second cleavage enzyme. We found that MNC2 and MNE6 recognized a MUC1* generated by cleavage of MUC1 by MMP9 but not by cleavage by other enzymes such as MMP2 (FIG. 37 and FIG. 75). We note that MMP9 is overexpressed in cancers and is a predictor of poor prognosis (vant Veer et al 2002; Dufour et al 2011) and has been implicated in metastasis (Owyong et al, 2019), whereas MMP2 is expressed in bone marrow. One antibody binding to a MUC1* generated by cleavage by a first enzyme but not by cleavage by a second enzyme implies that the antibody recognizes a conformational epitope rather than a linear epitope.

We reasoned that the most cancer specific antibodies would be those antibodies that are characterized by some combination of most or all of the following:
(i) Antibody binds to PSMGFR peptide;
Antibody does not bind to full-length MUC1;
(ii) Antibody binds to N-10;
(iii) Antibody does not bind to C-10;
(iv) Antibody competitively inhibits binding of NME1 or NME7_{AB} to MUC1* extra cellular domain or a PSMGFR peptide;
(v) Antibody recognizes a MUC1* generated by cleavage by MMP9;
(vi) Antibody recognizes a conformational epitope not a linear epitope.

### MNC2 and MNE6 are Cancer Specific

Our experiments show that both MNC2 and MNE6: a) Bind to tumor cells; b) monovalent forms block tumor growth in vitro and in vivo; c) have minimal to no binding of normal tissue while having robust binding to a wide panel of tumor tissues; d) when incorporated into CAR T cells, MNC2 and MNE6 directed CAR T cells do not recognize full-length MUC1 and do not kill cells that only express full-length MUC1; e) MNC2 and MNE6 directed CAR T cells cluster then kill tumor cells expressing MUC1*; and f) MNC2 and MNE6 recognize a MUC1 cleavage product when it is cleaved by MMP9.

MNC2 directed CAR T cells do not recognize normal, healthy cells that are MUC1* positive. A panel of normal cell lines, as well as primary cells, were co-cultured with huMNC2-CAR44 T cells. The normal cell populations were analyzed to determine whether or not the MNC2 directed CAR T cells killed them. The CAR T cells were analyzed to see if co-culture with the MUC1 positive normal cells activated the killing function of the CAR T cells, as measured by secretion of IL-2 or interferon gamma. As FIGS. 50-52 show, the MNC2 directed CAR T cells did not kill the normal cells, nor was there cytokine secretion, indicative of T cell activation. In addition, over 2,000 human tissue specimens were analyzed. The results showed that neither MNC2 nor MNE6 showed any significant binding to normal tissues but showed robust staining of a wide panel of cancerous tissues. For example, MNC2 stained 93% breast cancer specimens, 83% ovarian, 78% pancreatic and 71% lung cancer specimens. In addition, patient-matched primary tumors (FIG. 54) and subsequent metastases showed that the amount of MNC2-reactive MUC1* increased with tumor progression and metastases. In summary, MNC2 is a highly cancer specific antibody.

### Characterization of MNC2 and MNE6

Our gold standard, cancer-specific antibodies MNC2 and MNE6: 1) bind to N-10 peptide but not to the C-10 peptide; 2) compete with NME7_{AB} and dimeric NME1 for the same binding site near the C-terminus of the PSMGFR peptide, which is the membrane proximal portion of MUC1* on cells; 3) do not work in a Western blot assay indicating that they recognize a conformational rather than linear epitope; 4) recognize a MUC1* generated when MUC1 is cleaved by MMP9; 5) do not bind to full-length MUC1 but only to the cleaved form, MUC1*, in model cell lines as well as cancer cell lines; 6) show little to no binding to normal tissues but robustly stain a wide variety of tumor tissues; and 7) share some consensus sequences in their Complementarity Determining Regions, CDRs.

In an effort to identify other antibodies that are highly cancer-specific, like MNC2 and MNE6, we subjected new antibodies to a set of seven (7) characterization experiments: 1) epitope binding assays; 2) functional assays such as the ability to displace activating growth factor NME7_{AB} or dimeric NME1 from binding to MUC1* peptides PSMGFR or N-10; 3) Western blots to determine whether or not the antibodies recognized a linear epitope versus a conformational epitope, in which case the antibodies would not work in a Western; 4) binding assays to see if the antibodies recognized a cleaved MUC1 that was dependent on cleavage by MMP9; 5) FACS analysis to measure the ability of the antibodies to recognize MUC1* positive cells but not full-length MUC1; and FACS analysis to measure the ability of the antibodies to recognize MUC1/MUC1* positive cancer cells; 6) immunohistochemistry, IHC, assays of normal tissues versus cancerous tissues to determine true cancer specificity; and 7) aligning antibody sequences to determine if subsets of antibodies shared consensus sequences that could predict their cancer specificity or lack thereof.

Monoclonal antibodies were produced by immunizing animals with peptides derived from a MUC1 that is devoid of tandem repeats. These antibodies included PSMGFR and peptides that were extended at the N-terminus of PSMGFR. Immunizing peptides were:
PSMGFR (SEQ ID NO:2))
N+9/C-9 (9 amino acids added onto the N-terminus and 9 amino acids deleted from the C-terminus) (SEQ ID NO:824)
N+20/C-27 (20 amino acids added onto the N-terminus and 27 amino acids deleted from the C-terminus) (SEQ ID NO:823)

These monoclonal antibodies were then tested to determine which satisfied the seven (7) characterization criteria cited above, which we reasoned would identify the most cancer specific antibodies.

### Epitope Binding Assays

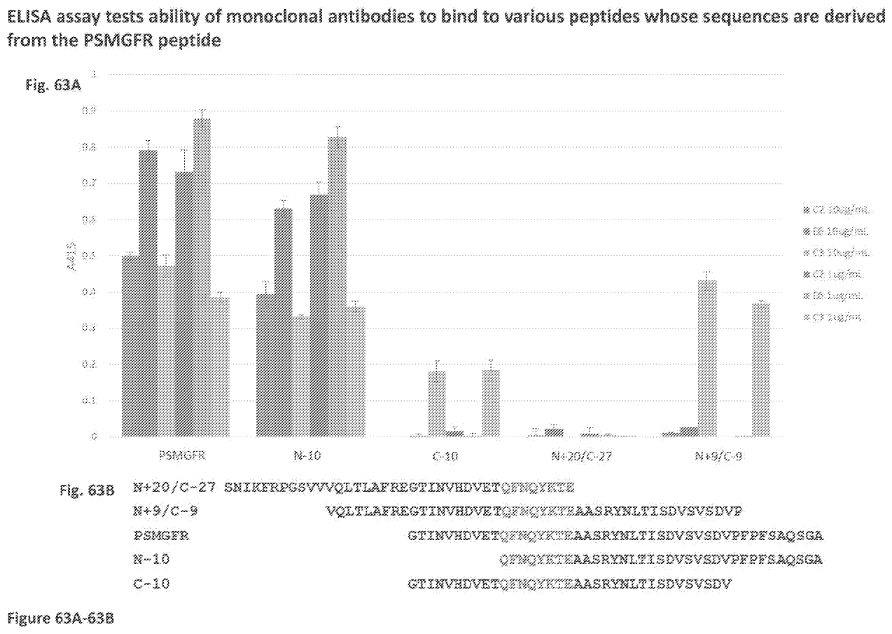

ELISA assays were performed to determine if, in addition to recognizing their immunizing peptide, they recognized PSMGFR, N-10 or C-10. In addition, they were tested for their ability to bind to N+20/C-27, N+9/C-9. We first did the ELISA assay on our set of reference antibodies, MNC2, MNE6, which we know are cancer-specific plus MNC3, which we know recognizes stem cells and progenitor cells (FIG. 63A-63B). None of the reference antibodies bound to the N+20/C-27 peptide. MNC2 and MNE6 cannot bind to PSMGFR peptides with 27, 10 or 9 C-terminal deletions, however, MNC3 binds to C-10 and to N+9/C-9 peptides.

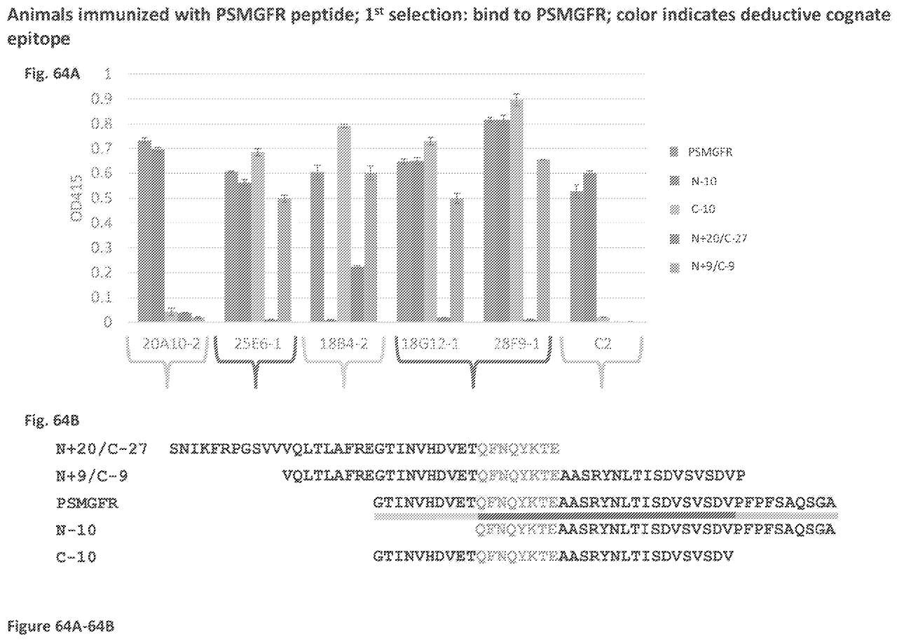

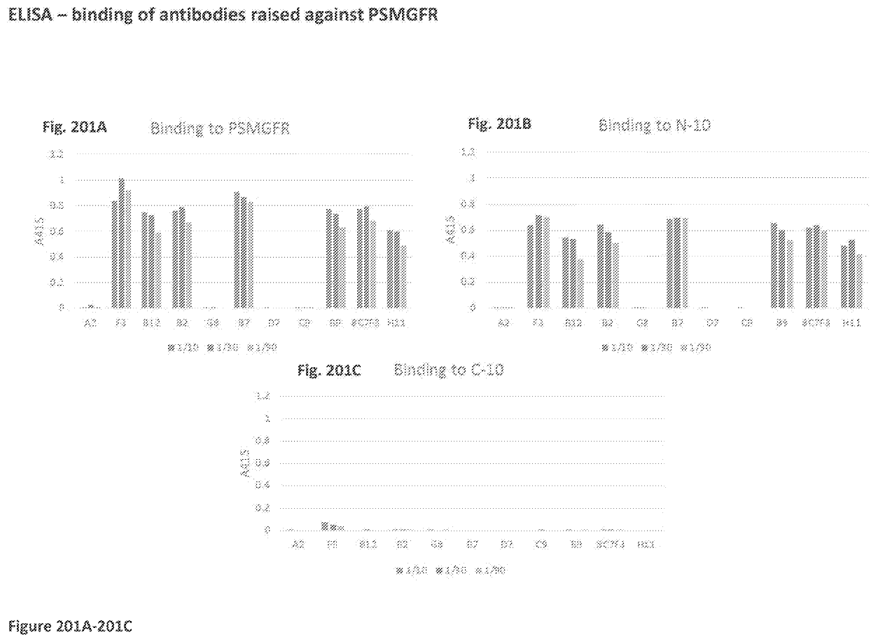

This same ELISA assay was performed on the antibodies of the invention (FIGS. 64-66 and FIG. 201). The binding patterns of the antibodies that were generated by immunizing with the PSMGFR peptide are shown in FIGS. 64A-64B. Note that only 20A10 exactly matches the binding profile of MNC2 and MNE6. 25E6, 28F9 and 18G12 are all able to bind to the N-10 peptide. 18B4 is the only antibody raised against the PSMGFR peptide that requires the 10 most N-terminal amino acids of the peptide. The color of the bars for each antibody in the ELISA graph are color coded to match the deductive cognate sequence, or a portion thereof, of that antibody. In addition, another set of antibodies was assayed by ELISA (FIG. 201). Of this set, B12, B2, B7, B9, 8C7F3, and H11 bound to the PSMGFR peptide, bound to the N-10 peptide, but not to the C-10 peptide (FIG. 201). The binding patterns of the antibodies that were generated by immunizing with the N+20/C-27 peptide are shown in FIG. 65A-65B. Although these antibodies were raised against the N+20/C-27 peptide, all but one, 45C11, still bind to the PSMGFR peptide, albeit at the N-terminal portion of PSMGFR. The binding of 45C11 is weak but deductive reasoning shows that all or some of the cognate epitope must lie within SNIKFRPGSVV (SEQ ID NO:1744).

Of the antibodies generated by immunizing with the N+9/C-9 peptide, 8A9 and 17H6 do not bind to the PSMGFR peptide, so must bind to the 9 additional N-terminal amino acids. Antibodies 3C5 and 39H5 appear to bind to the 10 most N-terminal amino acids of the PSMGFR peptide.

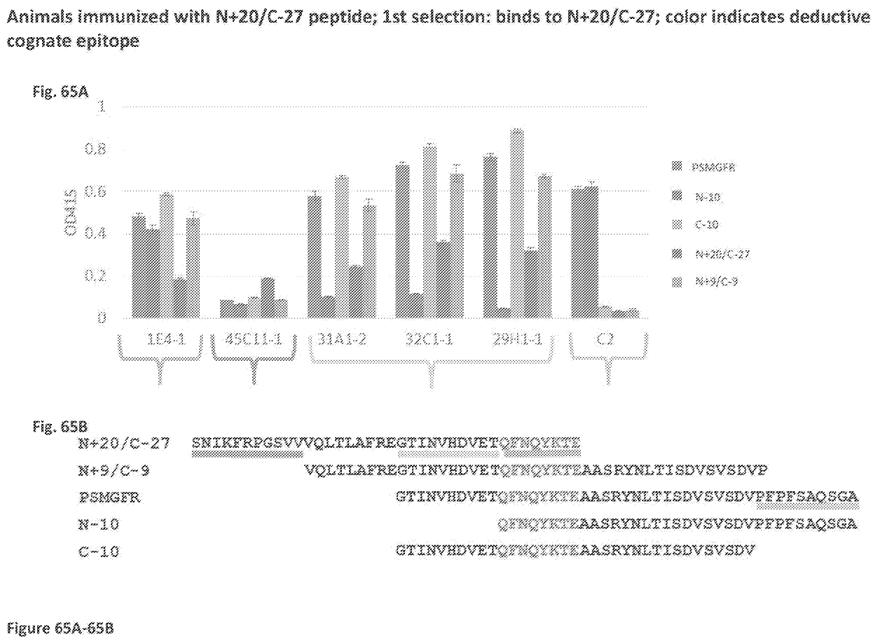

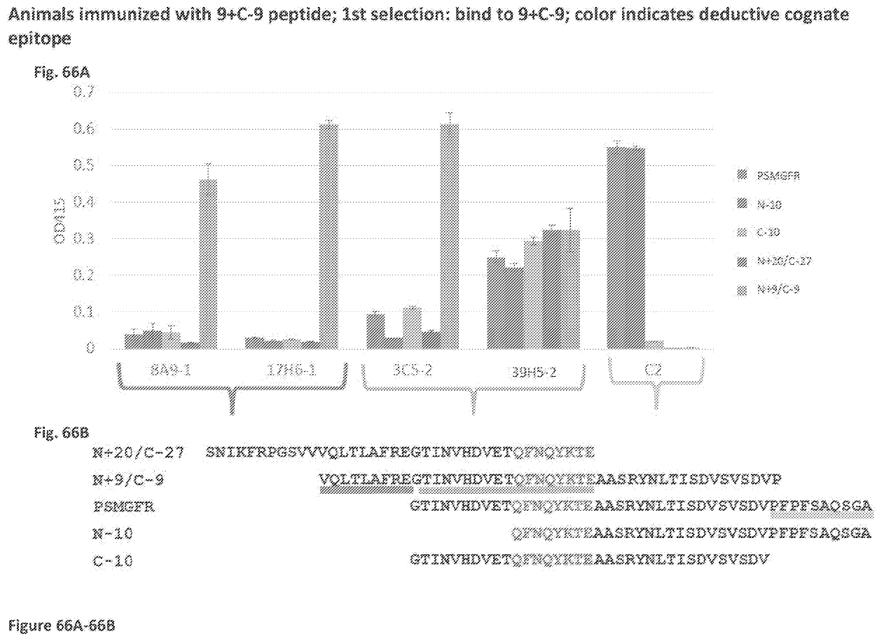

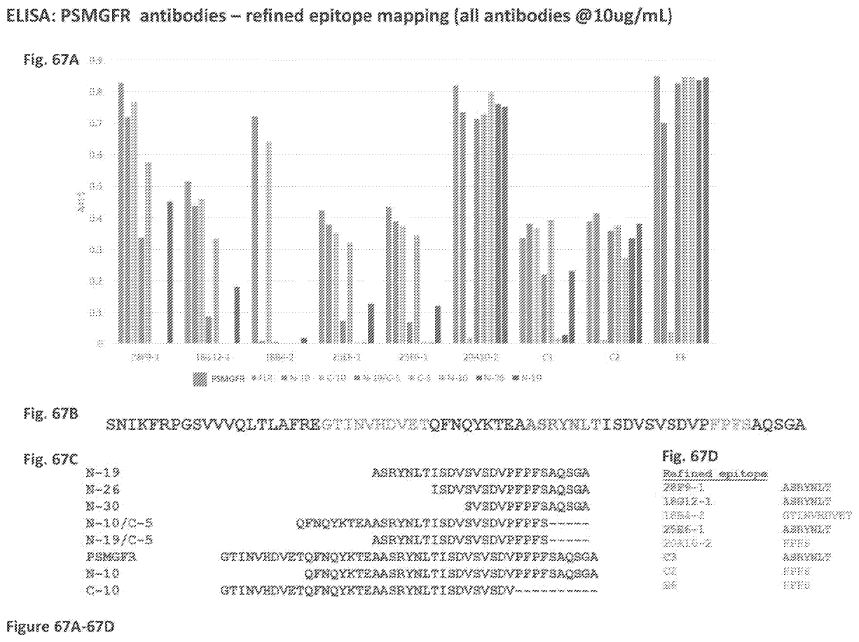

In order to further refine the epitopes to which each antibody binds, a series of smaller peptides derived from the PSMGFR sequence were synthesized: N-30 (SEQ ID NO:7), N-26 (SEQ ID NO:6), N-19 (SEQ ID NO:4), N-10/C-5 (SEQ ID NO:8), N-19/C-5 (SEQ ID NO:9). Each of the antibodies was tested in an ELISA assay for their ability to bind to this refined set of peptides, plus PSMGFR, N-10 and C-10 peptides (FIG. 67-69).

In FIG. 67A-67D, antibodies generated by immunization with the PSMGFR peptide were assayed. As can be seen in the figure, amino acids ASRYNLT (SEQ ID NO: 1745), which are essentially in the middle of the PSMGFR peptide, are important or essential for the binding of 28F9, 18G12, 25E6, and MNC3 antibodies. Amino acids GTINVHDVET (SEQ ID NO:1746), which comprise the most N-terminal part of the PSMGFR peptide are important or essential for the binding of the 18B4 antibody. Amino acids FPFS (SEQ ID NO:1747) are important or essential for the binding of 20A10, MNC2 and MNE6. We note that these three antibodies recognize a conformational epitope, not a linear epitope. Because the proline in the FPFS sequence significantly alters the conformation of nearby portions of the PSMGFR peptide, it is also possible that the antibodies do not bind directly to these four amino acids, but that the absence of the proline alters the fold of the remaining peptide such that the conformation to which 20A10, MNC2 and MNE6 bind, is no longer present.

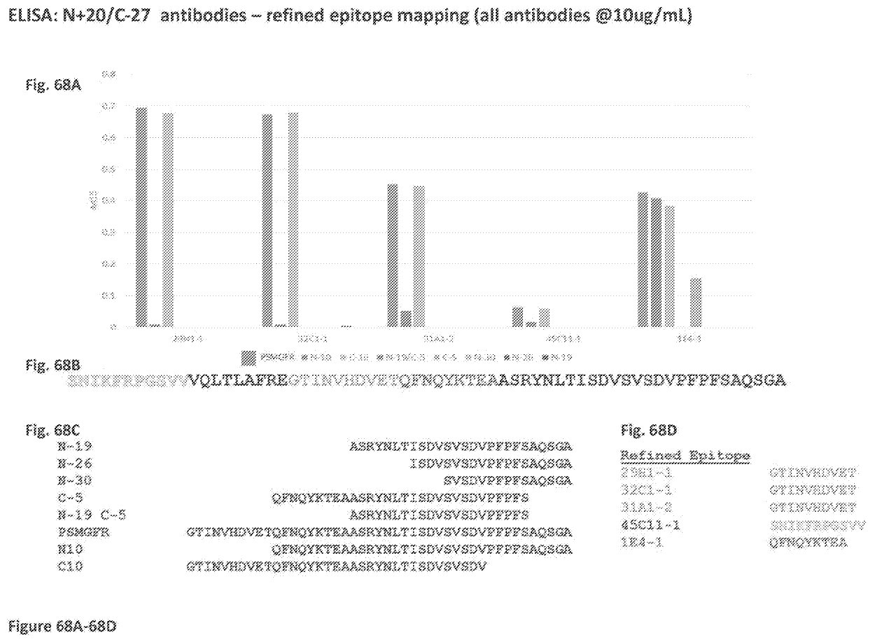

In FIG. 68A-68D, antibodies generated by immunization with the N+20/C-27 peptide were assayed. As can be seen in the figure, amino acids GTINVHDVET, which comprise the most N-terminal part of the PSMGFR peptide are important or essential for the binding of the 29H1, 32C1, and 31A1 antibodies. Amino acids SNIKFRPGSVVVQLTLAFRE (SEQ ID NO:1748), which is 20 additional amino acids N-terminal to the PSMGFR peptide and outside of the PSMGFR peptide, are important or essential for the binding of antibody 45C11. However, referring back to FIG. 65, antibody 45C11 was not able to bind to the N+9/C-9 peptide, therefore we conclude that amino acids within the SNIKFRPGSVV sequence are essential for the binding of 45C11. Amino acids QFNQYKTEA (SEQ ID NO:1749), which are still within the sequence of PSMGFR, are important or essential for the binding of antibody 1E4.

In FIG. 69A-69D, antibodies generated by immunization with the N+9/C-9 peptide were assayed. As can be seen in the figure, amino acids GTINVHDVET, which comprise the most N-terminal part of the PSMGFR peptide are important or essential for the binding of the 39H5 and 3C5 antibodies. As can be seen in the figure, amino acids VQLTLAFRE (SEQ ID NO: 1750), which is 9 additional amino acids N-terminal to the PSMGFR peptide and outside of the PSMGFR peptide, are important or essential for the binding of antibodies 17H6 and 8A9. Because the 17H6 and 8A9 antibodies do not bind to any of the smaller peptides shown in this figure, refer to FIG. 66A-66C, which shows that these two antibodies only bind to the peptide that has 9 additional amino acids N-terminal to the PSMGFR peptide.

Table 2 below lists antibodies of the invention and their cognate epitopes.

TABLE 2

| Immunizing | Antibody | Cognate |
|---|---|---|
| Peptide | Name | Sequence |
| PSMGFR | MNC2 | FPFS or PFPFSAQSGA |
| | MNE6 | FPFS or PFPFSAQSGA |
| | 20A10 | FPFS or PFPFSAQSGA |
| | 3C2B1 | FPFS or PFPFSAQSGA |
| | 5C6F3 | SVSDV |
| | MNC3 | ASRYNLT |
| | 25E6 | ASRYNLT |
| | 28F9 | ASRYNLT |
| | 18G12 | ASRYNLT |
| | 18B4 | GTINVHDVET |
| N + 20/C − 27 | 45C11 | SNIKFRPGSVV |
| | 29H1 | GTINVHDVET |
| | 32C1 | GTINVHDVET |
| | 31A1 | GTINVHDVET |
| | 1E4 | QFNQYKTEA |
| N + 9/C − 9 | 17H6 | VQLTLAFRE |
| | 8A9 | VQLTLAFRE |
| | 39H5 | GTINVHDVET |
| | 3C5 | GTINVHDVET |

### Ability to Displace NME7_{AB} Binding to the MUC1* Extra Cellular Domain Peptide PSMGFR

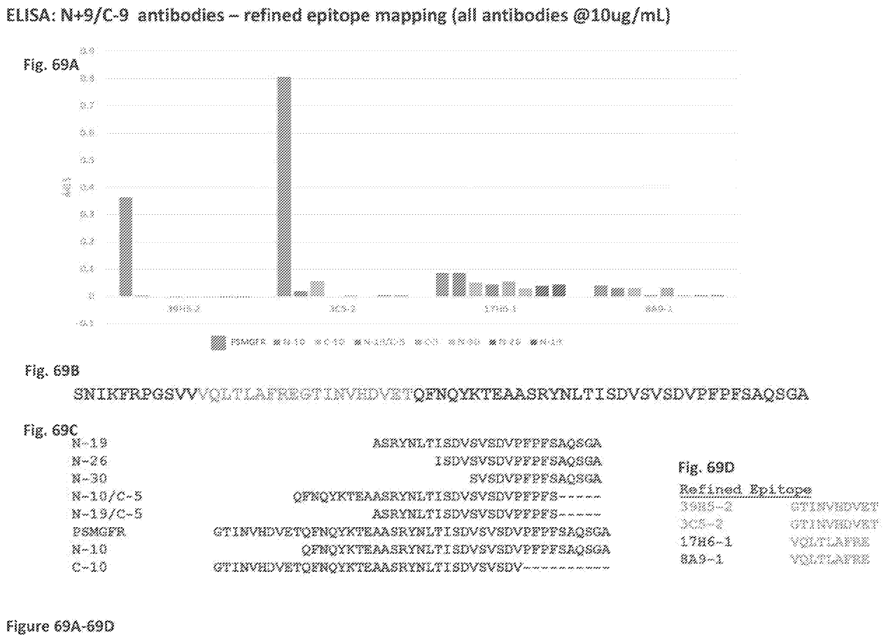

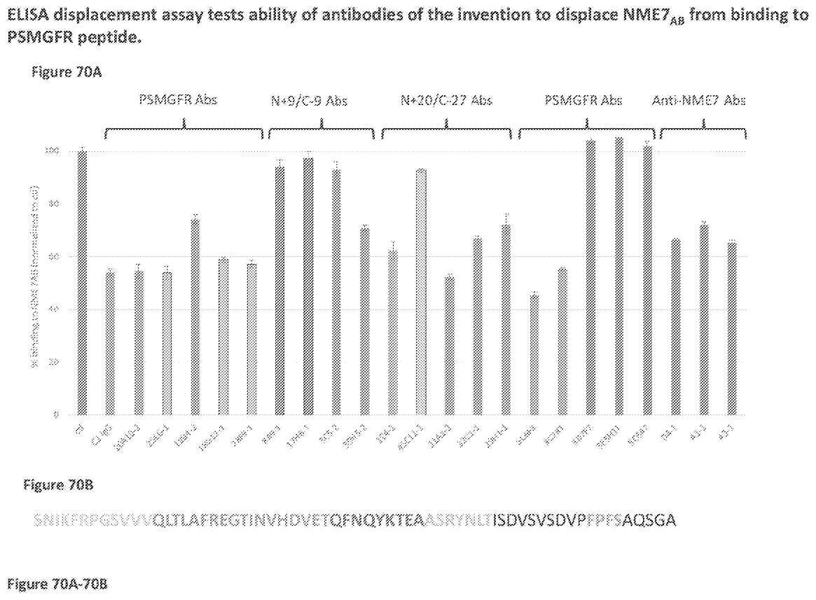

We previously reported that dimeric NME1 dimerizes MUC1* extra cellular domain and stimulates growth. Monomeric NME7_{AB} has two binding sites for MUC1* so that as a monomer it dimerizes MUC1* and mediates cancer cell growth. We showed that NME1 and NME7_{AB} can bind to the MUC1* extra cellular domain. In vitro, NME1 and NME7_{AB} bind to the PSMGFR peptide even if the 10 N-terminal amino acids are deleted, referred to herein as N-10 (SEQ ID NO:3). However, neither NME1 nor NME7_{AB} can bind to the PSMGFR peptide if the 10 membrane proximal amino acids are deleted, referred to herein as C-10 (SEQ ID NO:825). In summary, the epitope to which NME1 and NME7_{AB} bind includes all or part of the 10 membrane proximal amino acids: PFPFSAQSGA (SEQ ID NO: 1743). We tested various antibodies that were generated in animals by immunizing with the PSMGFR peptide for their ability to recognize cancer cells but not healthy cells. Among the most cancer selective were the MNC2 and MNE6 monoclonal anti-MUC1* antibodies. Two other monoclonal antibodies that were generated from immunizing animals with the PSMGFR peptide are MNC3 and MNC8. Although MNC2, MNE6, MNC3 and MNC8 all bind to the PSMGFR peptide, like NME1 and NME7a, MNC2 and MNE6 bind strongly to the N-10 peptide but not to the C-10 peptide. In fact, MNC2 and MNE6 competitively inhibit the binding of NME1 and NME7A to PSMGFR. Conversely, MNC3 and MNC8 are able to bind to the C-10 peptide, bind less well to the N-10 peptide and do not compete with NME1 nor NME7A for binding to MUC1* peptides, including PSMGFR (FIG. 70). MNC3 and MNC8 are less cancer specific than MNC2 and MNE6. MNC3 and MNC8 recognize stem and progenitor cells, such as hematopoietic stem cells, whereas MNC2 and MNE6 do not. Because hematopoietic stem cells are the progenitor cells for the blood cells, it would be problematic to have a cancer therapeutic that would also target such an important normal cell type.

In this experiment, antibodies of the invention were tested for their ability to displace NME7A from binding to the PSMGFR peptide. In this experiment, a multi-well plate was coated with the PSMGFR peptide. Recombinant NME7A was allowed to bind to the surface-immobilized PSMGFR peptide. Wash steps followed. Various antibodies were added, followed by wash steps. The amount of NME7A that remained attached to the PSMGFR coated plate, after antibody competition, was measured by detecting a tag on the NME7A. As a control, anti-NME7A antibodies were also tested for their ability to displace NME7A from the PSMGFR. FIG. 70 shows a graph of an ELISA displacement assay. The bar graph is color coded to indicate the cognate epitope to which each antibody binds. As can be seen in the figure, the antibodies that bind to the more C-terminal portions of PSMGFR are the most potent at disrupting the binding of onco-embryonic growth factor NME7A to the MUC1* extra cellular domain or the PSMGFR peptide. The rank order of potency for disrupting binding of NME7_{AB} to PSMGFR according to their cognate epitope is as follows: FPFS>ASRYNLT>QFNQYKTEA>GTINVHDVET. Antibodies that bind to epitopes outside of the PSMGFR peptide, such as 45C11, 8A9 and 17H6 did not compete with NME7_{AB} for binding.

### Western Blot Assay to Determine Linear Versus Conformational Cognate Epitope

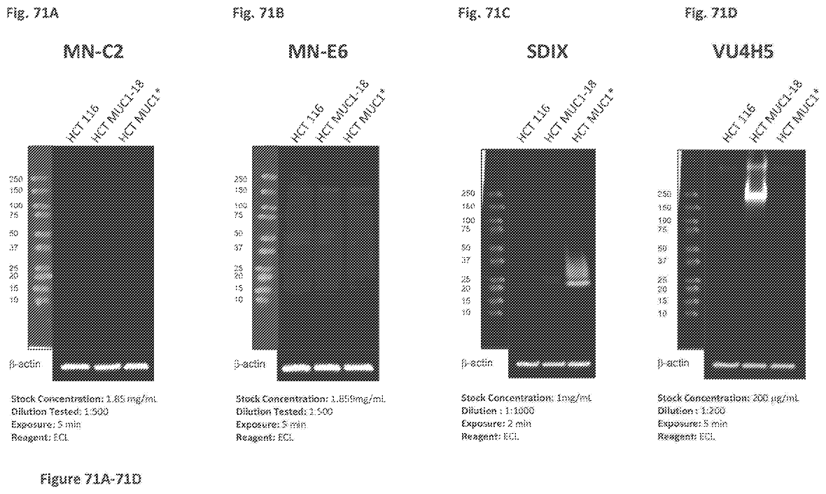

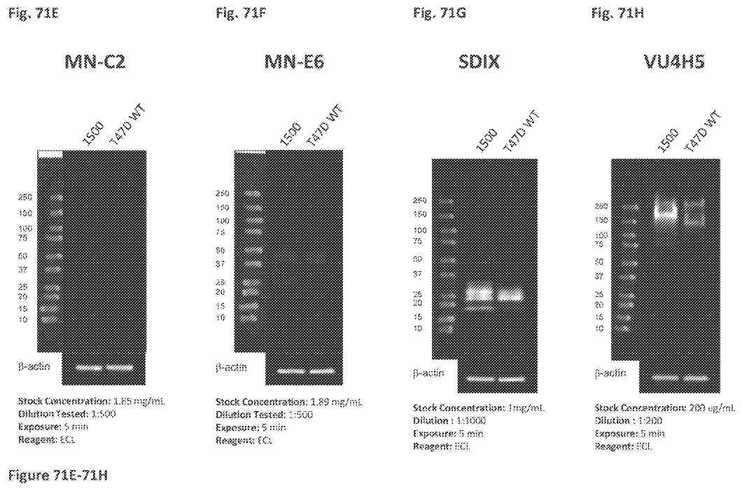

Antibodies were tested to determine whether they recognize a linear or a conformational epitope. Only antibodies that recognize a linear epitope work in Western blots when using denaturing gels. For comparison, known antibodies were tested for their ability to bind to HCT-116, a MUC1 negative cancer cell line, HCT-MUC1-18, which is a cleavage resistant clone of HCTs transfected with full-length MUC1, and HCTs transfected with MUC1*, wherein the extra cellular domain comprises only the PSMGFR sequence. The antibodies tested for comparison are MNC2 and MNE6, which were known to only recognize a conformational epitope, SDIX which is a polyclonal antibody raised against PSMGFR and VU4H5, which is a commercially available monoclonal antibody that recognizes the tandem repeats of full-length MUC1 (FIG. 71A-71D). As can be seen, neither MNC2 nor MNE6 recognize a MUC1 or MUC1* specific linear epitope. FIGS. 71A and 71E show that antibody 20A10 also does not recognize a MUC1 or MUC1* specific linear epitope. The SDIX polyclonal antibody recognizes HCT-MUC1* but not full-length MUC1 and VU4H5 only recognizes full-length MUC1. These same antibodies were also tested for their ability to work in Western blots of two breast cancer cell lines 1500, aka Zr-75-1, and T47D cells and show the same binding pattern (FIG. 71E-71H).

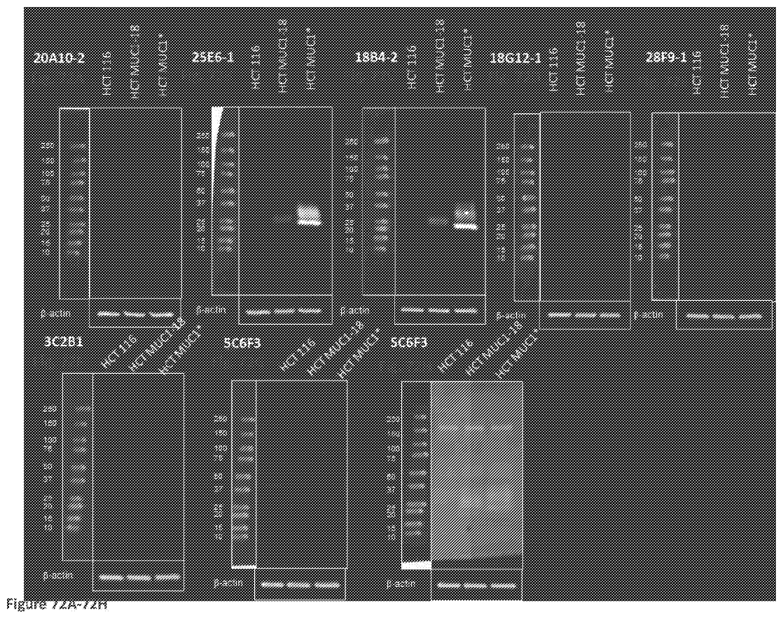

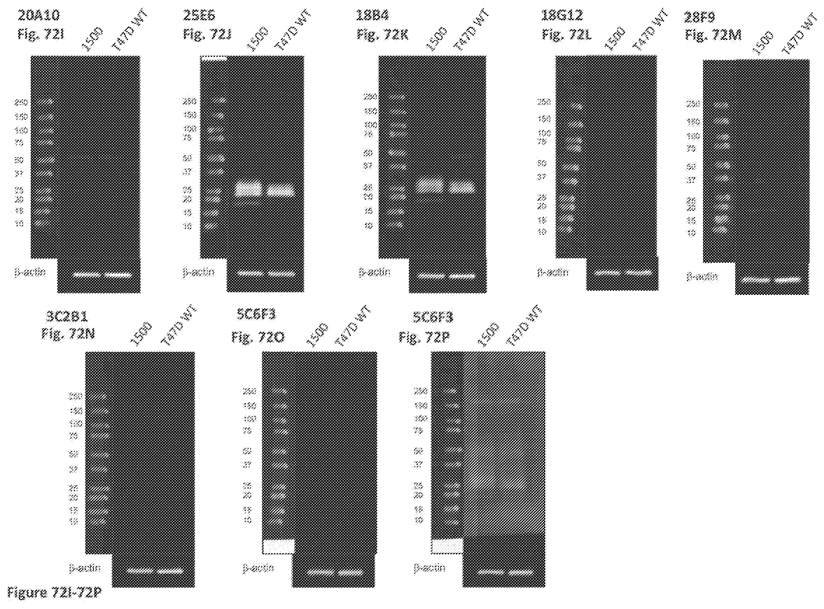

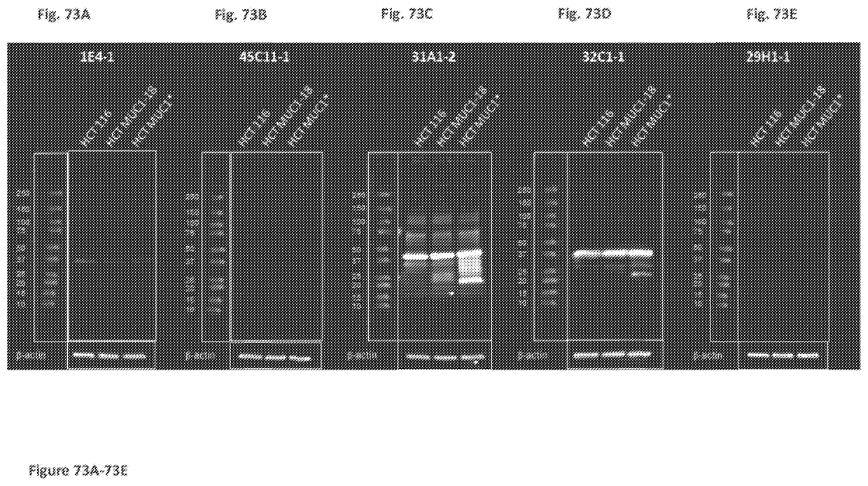

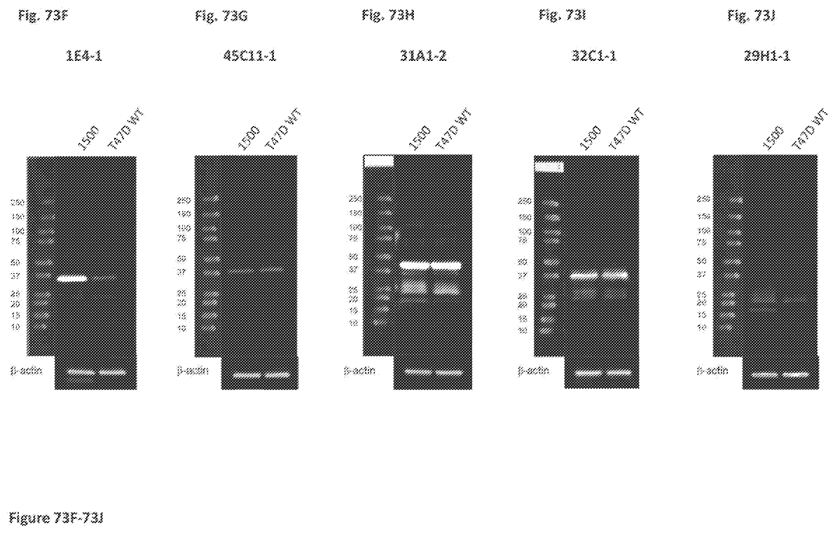

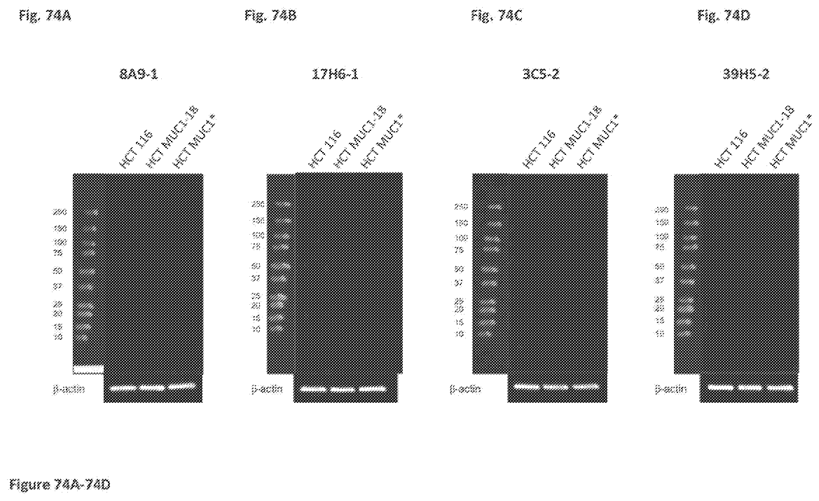

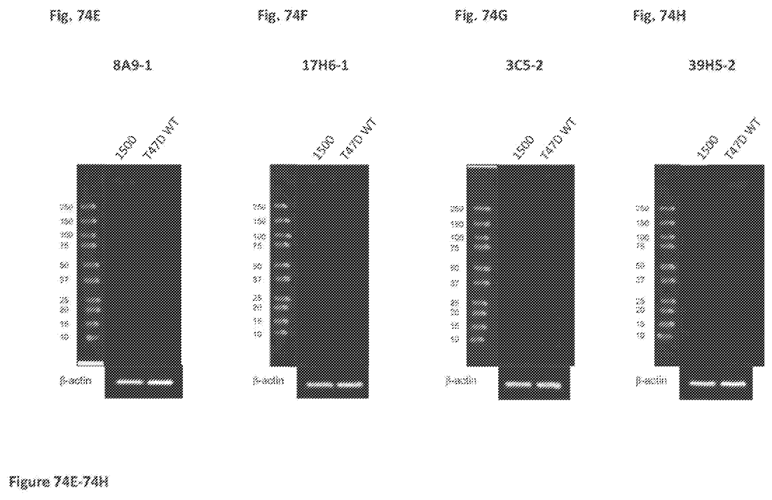

Antibodies that were raised against the PSMGFR peptide were tested the same way in Western blots (FIG. 72A-72P). As can be seen, antibodies 25E6 and 18B4 recognize linear epitopes but 20A10 (FIG. 72A; 72I), 3C2B1 (FIG. 72F; 72N), 5C6F3 (FIG. 72G; 72O), 18G12 and 28F9 do not, indicating that they bind to a conformational epitope. Antibodies that were raised against the N+20/C-27 peptide were tested the same way in Western blots (FIG. 73A-73J). As can be seen, antibodies 31A1 and 32C1 recognize linear epitopes. Antibodies 1E4 and 45C11 may recognize a conformational epitope. Antibodies that were raised against the N+9/C-9 peptide were tested the same way in Western blots (FIG. 74A-74H). As can be seen, none of these antibodies recognize linear MUC1 or MUC1* specific epitopes. These antibodies may recognize a conformational epitope. However, an alternative interpretation is that the lack of binding in a Western blot means that they do not specifically recognize MUC1 or a MUC1 cleavage product or that the concentration used in this assay was insufficient.

Recognition of a MUC1 Cleavage Product after Cleavage by MMP9

We previously demonstrated that MNC2 recognizes a MUC1* that is generated when full-length MUC1 is cleaved by matrix metalloprotease 9, MMP9 (FIG. 37). MMP9 is expressed by tumor tissues and is a predictor of poor prognosis for breast cancers (vant Veer et al 2002; Dufour et al 2011). MMP9 has also been implicated in metastasis (Owyong et al 2019). Recall also that MNC2 competitively inhibits the binding of onco-embryonic growth factor NME7_{AB} to the MUC1* extra cellular domain (FIG. 3). Therefore, it follows that onco-embryonic growth factor, which activates growth and survival functions of MUC1*, also recognizes a MUC1* generated by cleavage by MMP9. It then follows that the most cancer specific antibodies are those that recognize a conformational epitope formed when MUC1 is cleaved to MUC1* by MMP9.

Antibodies generated by immunization with PSMGFR, N+20/C-27, or N+9/C-9 were tested for their ability to recognize MUC1 after it is cleaved by MMP9. To do this, we transfected HCT-116, a MUC1 negative colon cancer cell line, with full-length MUC1 and isolated a single cell clone that is cleavage resistant; this cleavage resistant cell line is called HCT-MUC1-18. To HCT-MUC1-18 cells was added either a catalytically active MMP9 or MMP2. The enzymes, added over a range of concentrations, were incubated with the cells for 24 hours. The resultant cells were then incubated with the various antibodies and analyzed by FACS to determine which bound to a MUC1 cleavage product produced by cleavage by MMP9 (FIG. 75A-75N). Note that the first bar of each graph shows that none of the antibodies binds to full-length MUC1 in the absence of cleavage. Each bar graph is labeled with both the name of the antibody used in that assay and its cognate epitope. The order of the graphs from right to left corresponds to the distance from the cell surface of the antibody's cognate epitope. The antibodies that bind to the more C-terminal epitopes within PSMGFR peptide, such as 20A10 (FIG. 75E), 3C2B1 (FIG. 75O) and 5C6F3 (FIG. 75P), showed a concentration dependent increase in the binding to a MUC1 cleavage product after cleavage by MMP9 but not MMP2. Antibody 45C11, which binds to the SNIKFRPGSVV epitope, which is outside of the PSMGFR portion of MUC1, does not recognize a MUC1 cleavage product after cleavage by MMP9 or MMP2 (FIG. 75K). Similarly, antibodies 8A9 and 17H6 bind to the VQLTLAFRE epitope, which is also outside of the PSMGFR sequence, and they do not bind to a MUC1 cleaved by MMP9 or MMP2. This result is consistent with the idea that MMP9 cleaves MUC1 such that the extra cellular domain of the remaining transmembrane cleavage product comprises essentially the amino acids of the PSMGFR peptide. For the greatest degree of cancer specificity, the antibody should recognize a conformational epitope of a MUC1 cleavage product created when MUC1 is cleaved by MMP9. Of the antibodies shown in FIG. 75A-75N, only 20A10 recognizes the MUC1 cleavage product produced by cleavage by MMP9 and also does not work in a Western blot, indicating it recognizes a conformational epitope, as do MNC2 and MNE6. Cleavage and release of the massive tandem repeat domain of MUC1 unmasks the ectopic binding site on MUC1*; linear epitopes will be unmasked in addition to conformational epitopes.

### FACS Analysis of Binding to a Panel of Cancer Cell Lines

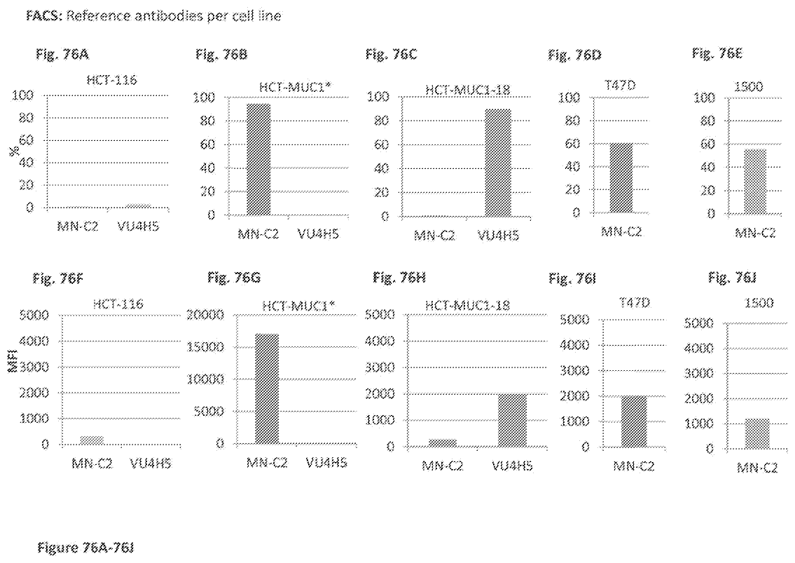

Fluorescence Activated Cell Sorting, FACS, was performed on reference antibodies as well as new antibodies of the invention. FACS analyses of reference antibodies MNC2, “C2”, and VU4H5 binding to either the MUC1-negative cell line HCT-116, HCTs transfected with MUC1*, “HCT-MUC1*”, a cleavage resistant single cell clone of HCTs transfected with MUC1 full-length, “HCT-MUC1-18”, and MNC2 binding to breast cancer cells line T47D or breast cancer cell line 1500 also known as ZR-75-1, was performed (FIG. 76A-76J). This analysis shows that MNC2 binds to an ectopic binding site on the extra cellular domain of MUC1*, which is only available after cleavage and release of the bulk of the extra cellular domain comprising the tandem repeat domain. VU4H5 binds to hundreds of repeating epitopes in the tandem repeat domain of full-length MUC1 and does not bind to MUC1*. Although we know that cancer cell lines express both full-length MUC1 and MUC1*, antibodies against full-length MUC1 have, as yet, been shown to have no therapeutic value. Stimuvax, ImMucin, IMGN242, SAR566658, PankoMab and AS1402 were all antibodies that bound to full-length MUC1 and all failed to show efficacy in clinical trials. MUC1*, and not full-length MUC1, is a potent growth factor receptor that mediates the growth of cancer cells (Mahanta et al 2008) and their resistance to chemotherapy agents (Fessler et al 2009). These studies showed that full-length MUC1 had no tumor promoting activity. Further, IHC studies show that as tumor stage increases, the amount of MUC1* increases as the amount of full-length MUC1 decreases (FIG. 54). In fact, studies with tissue micro arrays of breast cancers show that nearly 30% of breast cancer specimens had no detectable full-length MUC1, compared to only 5% that were negative for MUC1* (FIG. 10-11). A point to consider for therapeutics that target full-length MUC1 is that if cells expressing full-length MUC1 are eliminated, that would simply enrich the tumor population for the more virulent MUC1* growth factor receptor expressing cells, which would make the cancers worse.

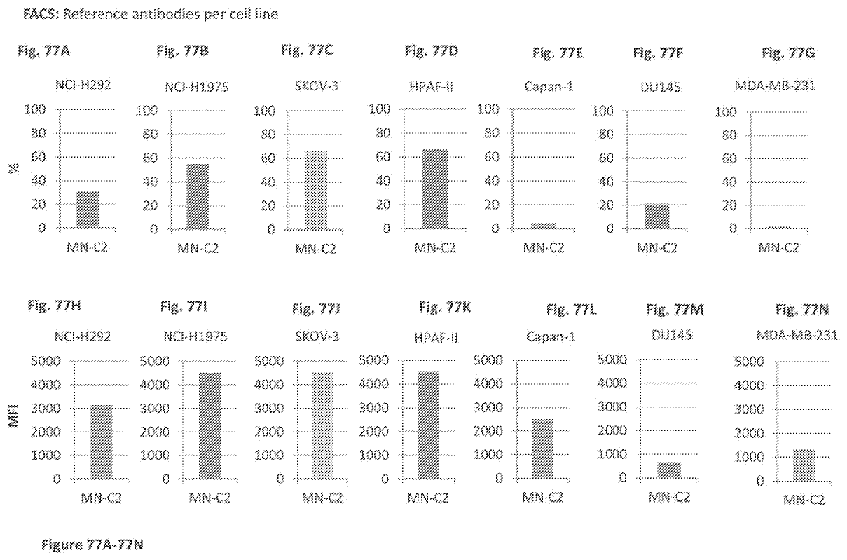

Reference antibody MNC2, “C2”, was analyzed by FACS for its ability to bind to a panel of cancer cell lines that are all MUC1* positive, with the exception of MDA-MB-231, which expresses MUC1 and MUC1* at a level that is so low that it is often used as a negative control (FIG. 77A-77N). The panel of cancer cells that was probed with MNC2 included T47D and 1500 breast cancer cells, NCI-H292 and NCI-H1975 lung cancer cells, SKOV-3 ovarian cancer cells, HPAF-II and Capan-1 pancreatic cancer cells, DU145 prostate cancer cells, and MDA-MB-231, breast cancer cells, which are nearly MUC1 and MUC1* negative. MNC2 robustly recognized a wide range of cancer cell lines. We note that although MNC2 recognized HPAF-II pancreatic cells, it did not recognize another pancreatic cell line, Capan-1, as well. Similarly, MNC2 did not recognize prostate cancer cell line DU145 very well. In IHC tissue studies, we found that MNC2 recognized about 57% of prostate cancer tissues and 78% of pancreatic tissues, albeit with significant tumor heterogeneity.

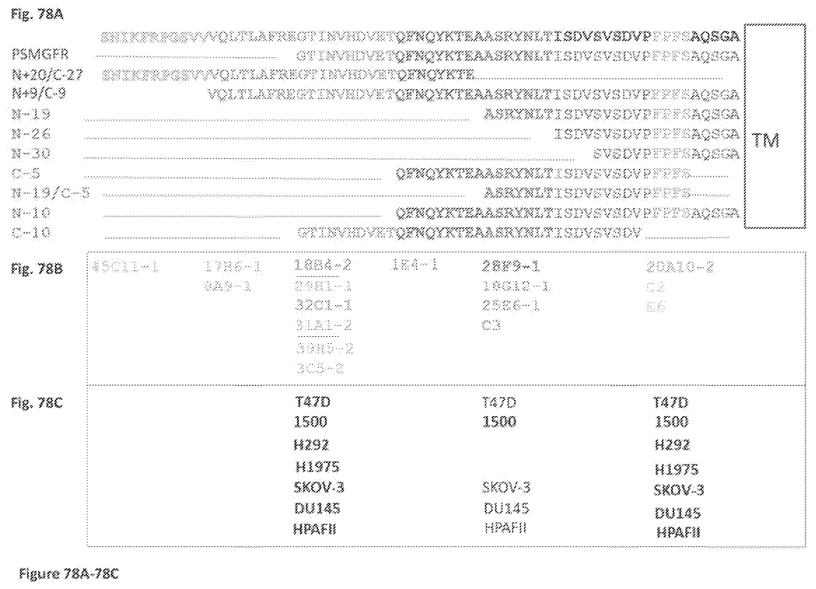

FIG. 78A-78C shows a color coded schematic of the PSMGFR sequence that has been extended or deleted at both the N- and C-termini. Antibodies of the invention were tested against this subset of peptides to further refine the epitopes to which each antibody binds or the critical amino acids within the epitope to which each antibody binds. FIG. 78A is an aligned schematic of the various subsets of peptides. FIG. 78B lists the antibodies that bind to each of the color coded sequences. FIG. 78C lists the cancer cell lines that each antibody recognizes.

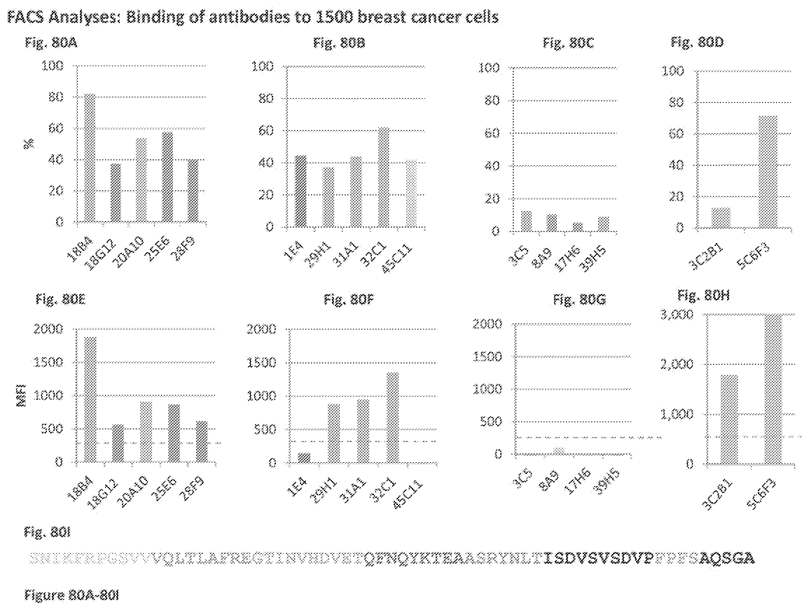

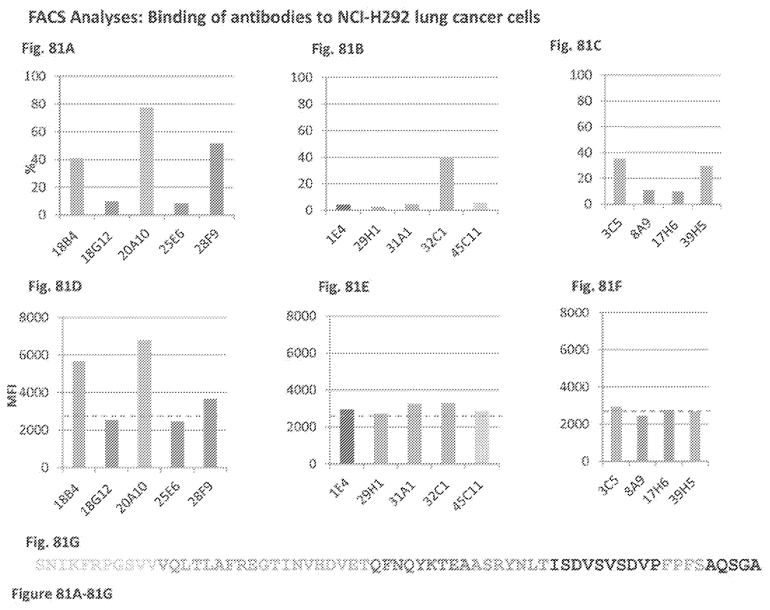

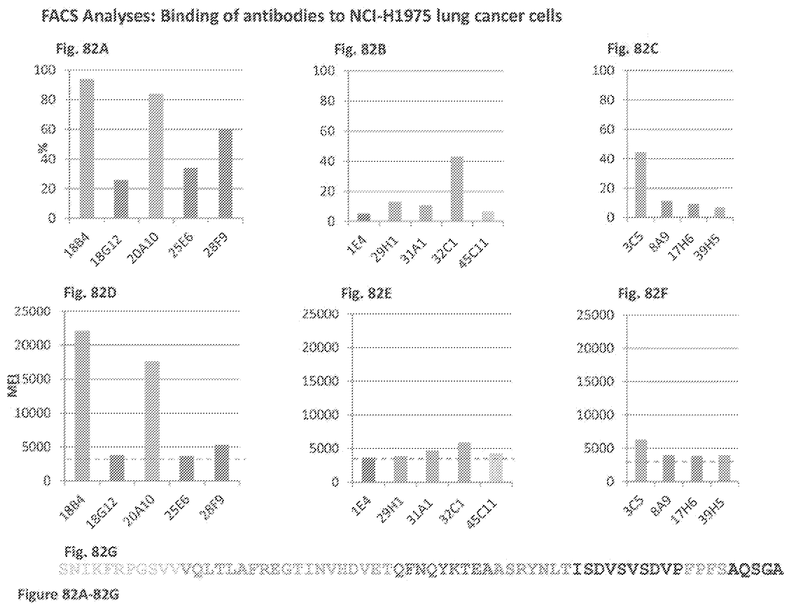

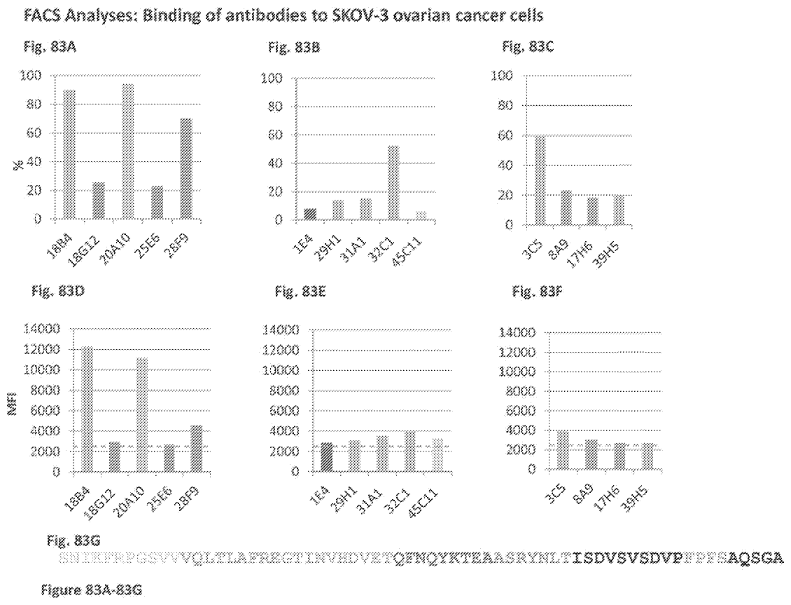

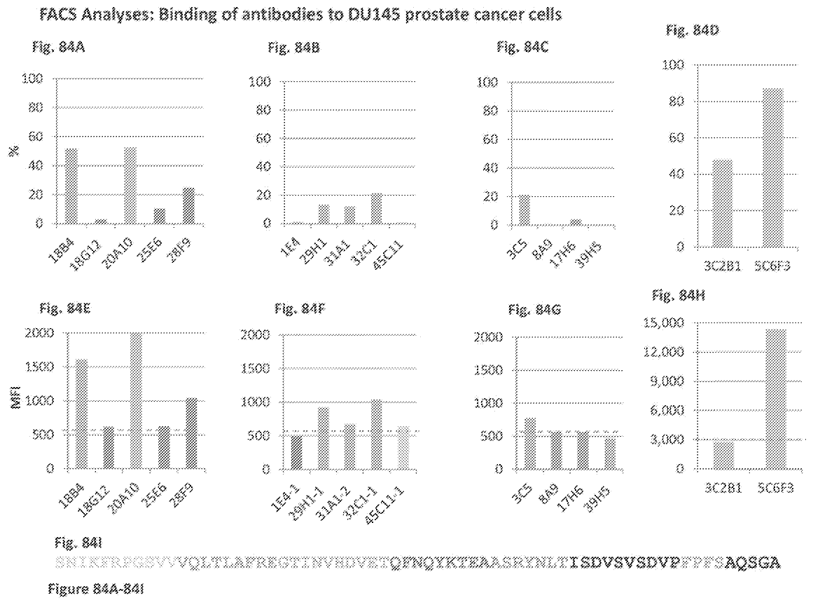

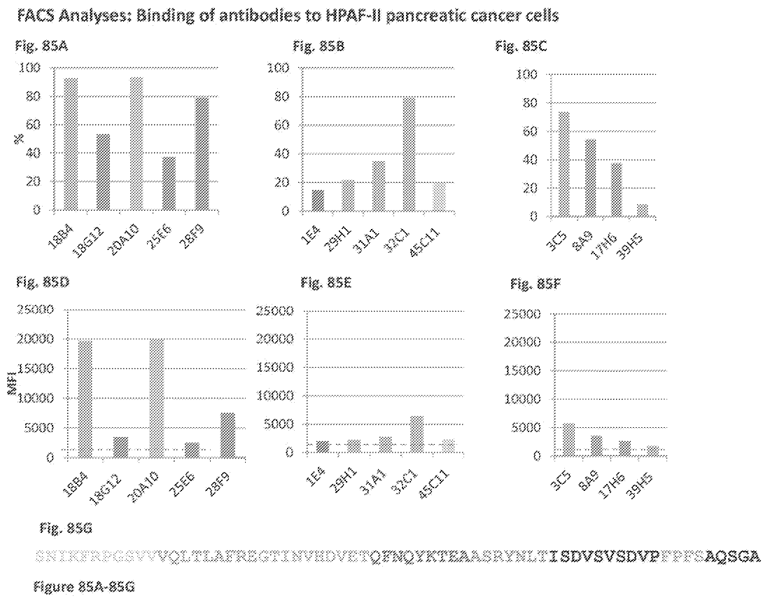

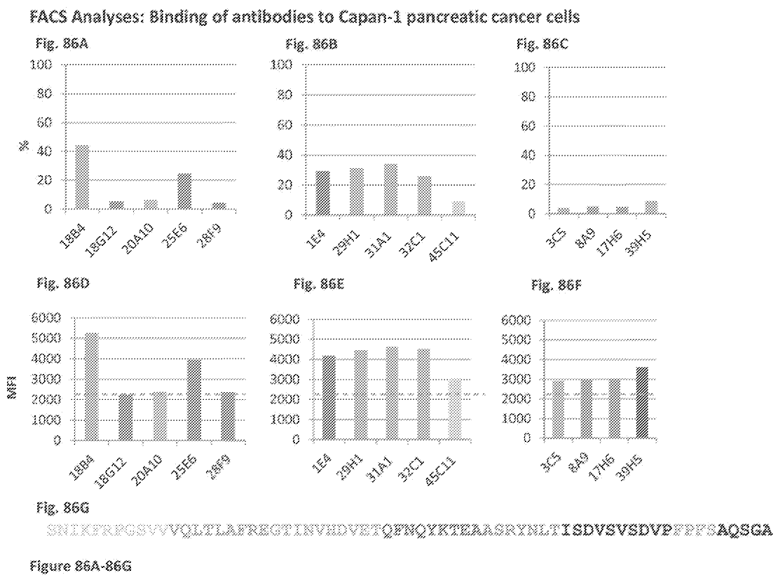

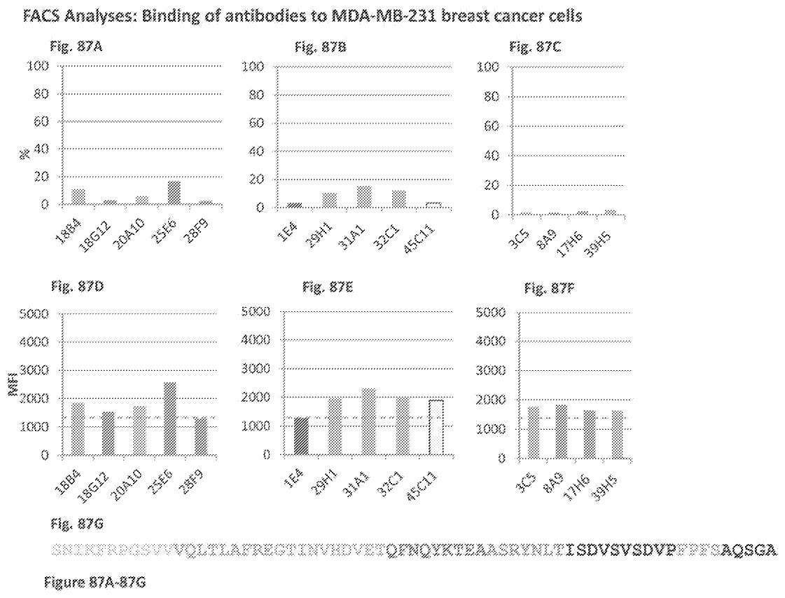

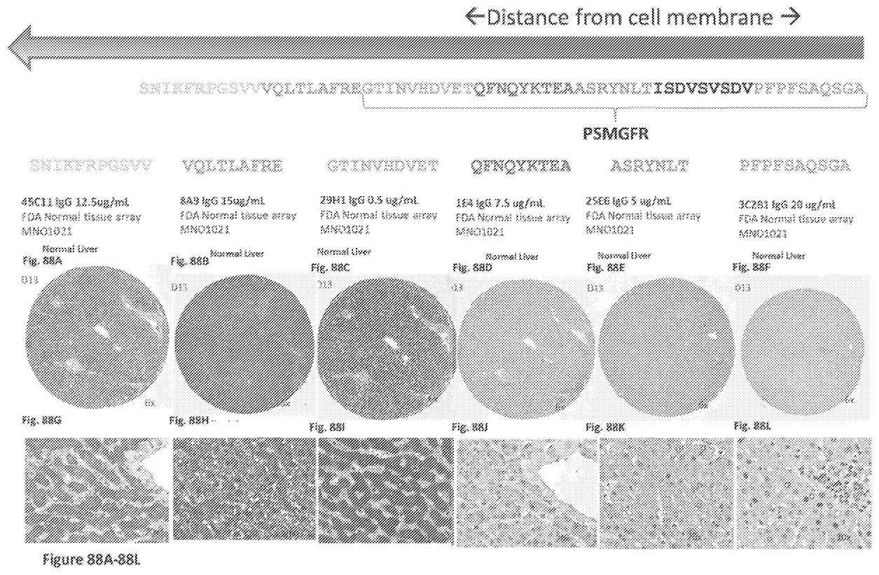

FIGS. 80-87 show graphs of FACS analyses wherein antibodies of the invention are compared for their ability to specifically recognize different types of cancer cells. Percent cells recognized as well as the Mean Fluorescence Intensity, MFI, was measured. Considering only these FACS experiments, they show that only antibodies that recognize the PSMGFR peptide are able to recognize cancer cell lines. Antibodies that bind to epitopes outside of the PSMGFR sequence do not specifically recognize these cancer cell lines.

### IHC Tissue Studies of Normal Versus Cancerous Tissues to Determine True Cancer Specificity

Immunohistochemistry, IHC, tissue studies of tissue micro arrays, “TMAs”, are a more stringent test of the cancer specificity of antibodies than FACS analysis of a single cancer cell line. Cancer cell lines are a single cell from a single patient that have been expanded in a lab for decades. Cell lines are limited in that they are not representative of a cross section of the human population. Further, after culturing the cell line in vitro for decades it may no longer look like the original cell. Also, there are no real normal cell lines for comparison, as they have to be made immortal. Tissue studies are more informative because each tissue micro array comprises tissues from multiple donors and the cells are in their natural environment, without years of culturing under non-physiologic conditions. Additionally, tissues provide information regarding tumor heterogeneity as well as information regarding normal patterns of expression. Each antibody of the invention was used to probe a normal tissue micro array, FDA Normal Array MNO1021. In addition the antibody was also used to probe a panel of cancerous tissue arrays. In some cases, antibodies that showed strong staining of normal tissues, especially of critical organs such as heart or lung, were tested on a limited number of cancerous tissue arrays, since their cross reactivity to normal tissues eliminated them from consideration as anti-cancer therapeutics.

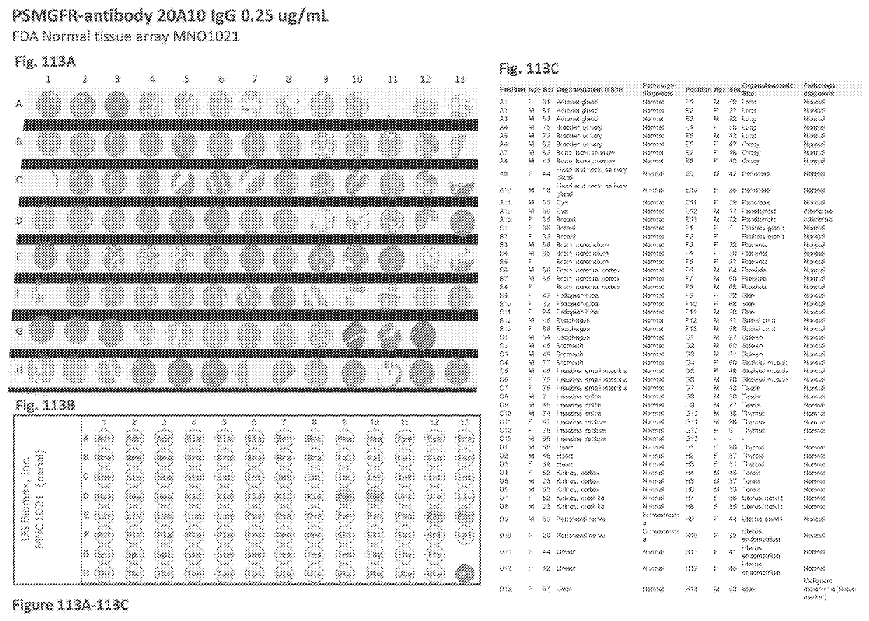

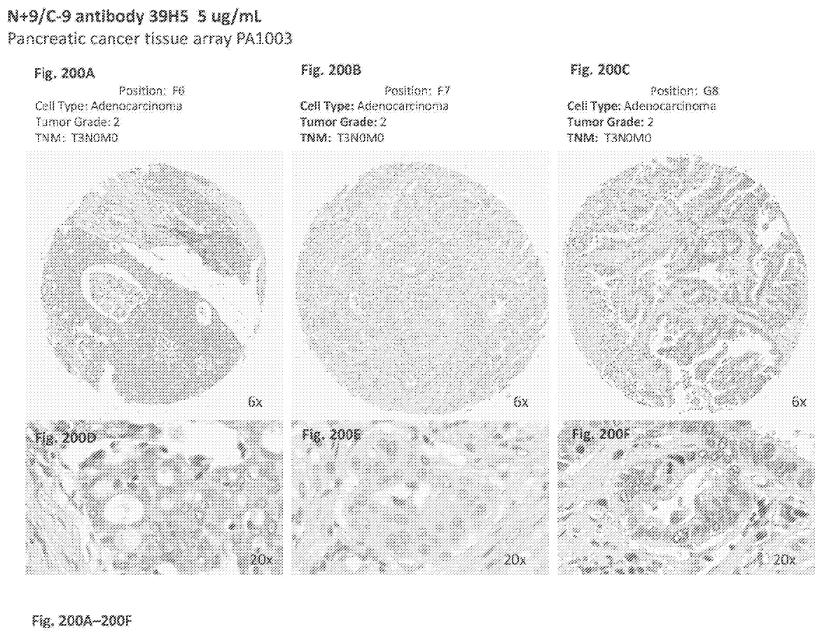

FIGS. 113-200 show photographs of the IHC staining of normal TMAs versus cancerous TMAs for each antibody of the invention.

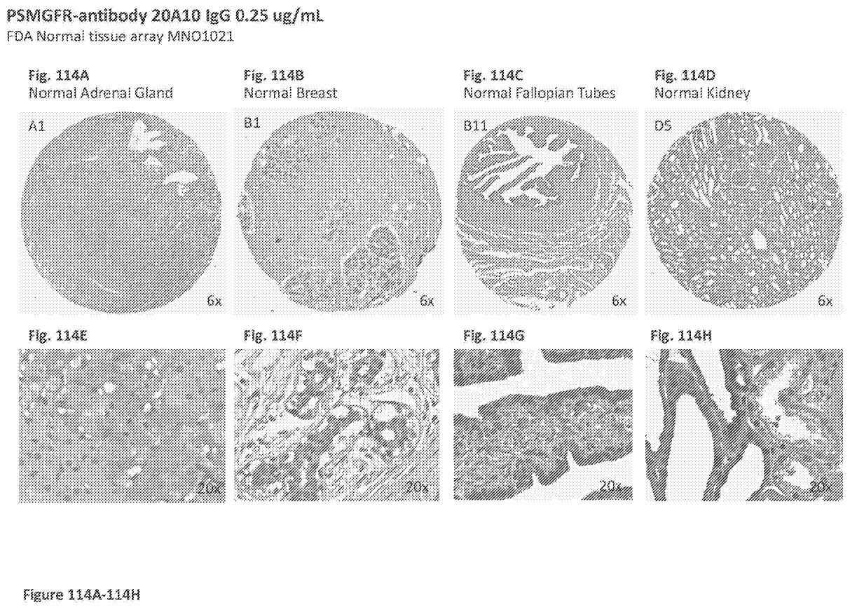

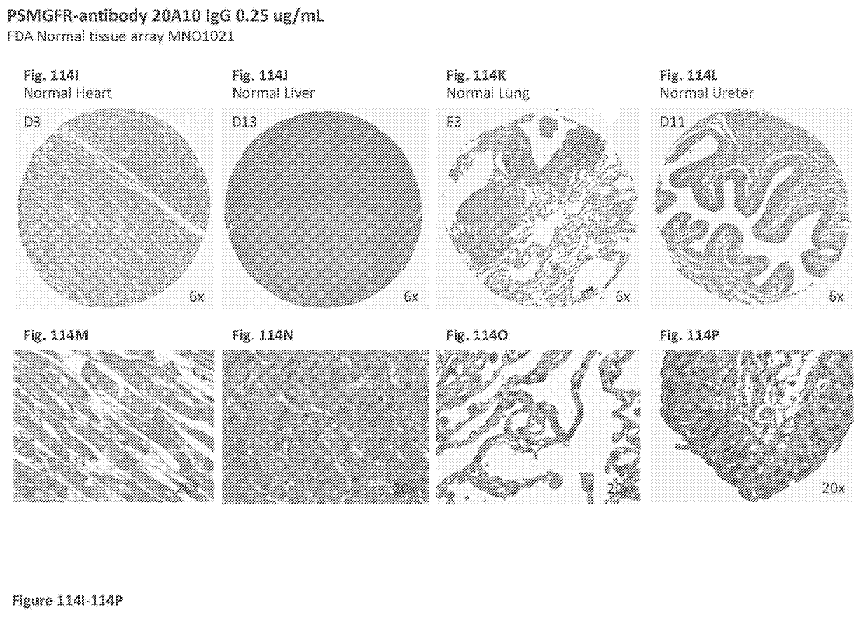

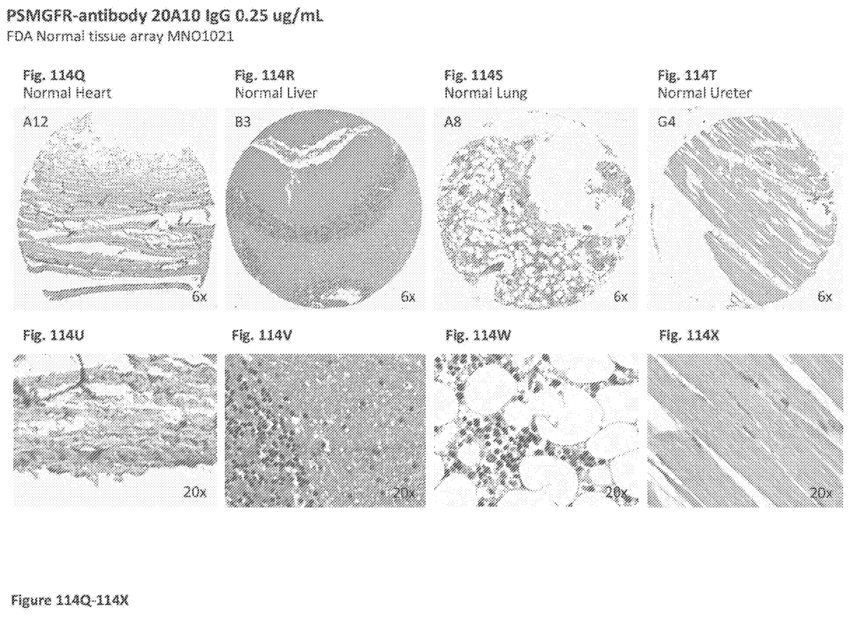

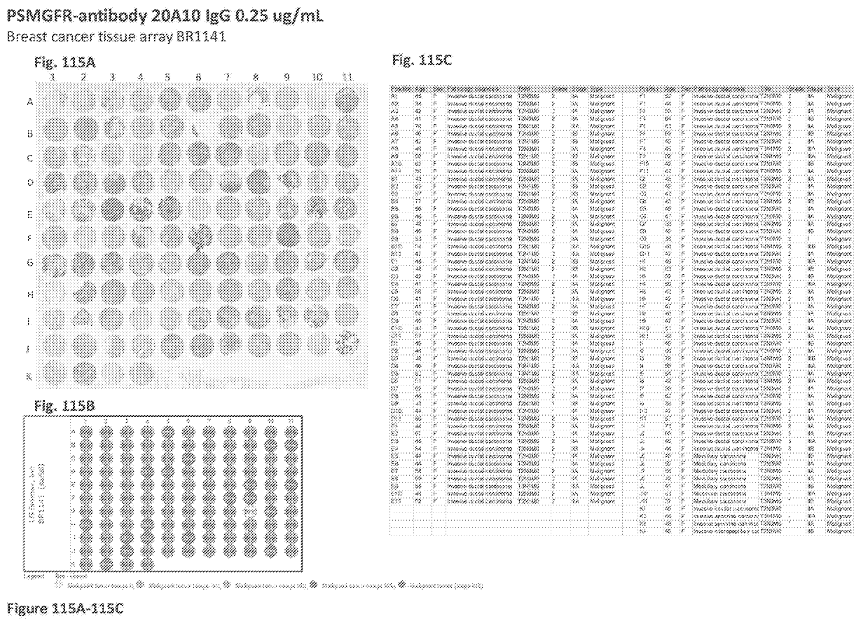

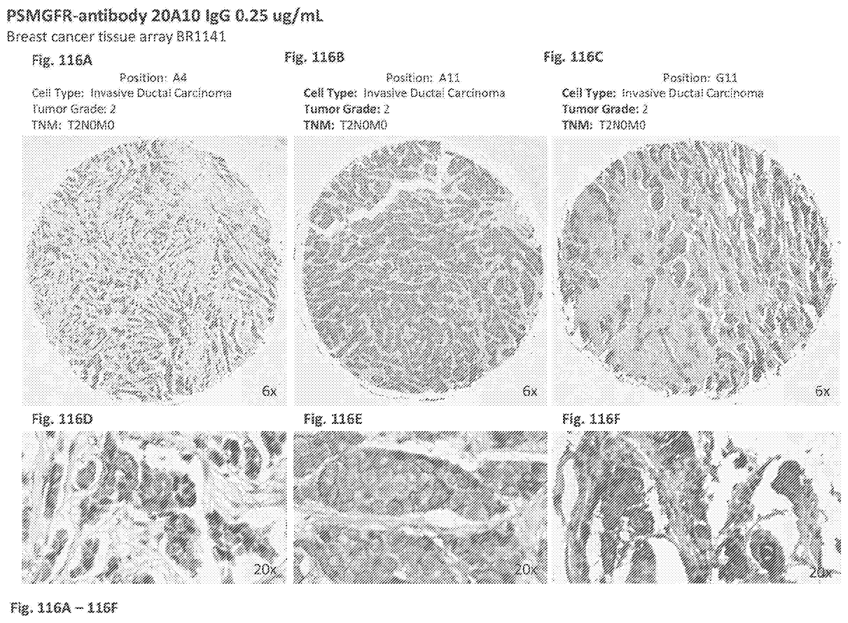

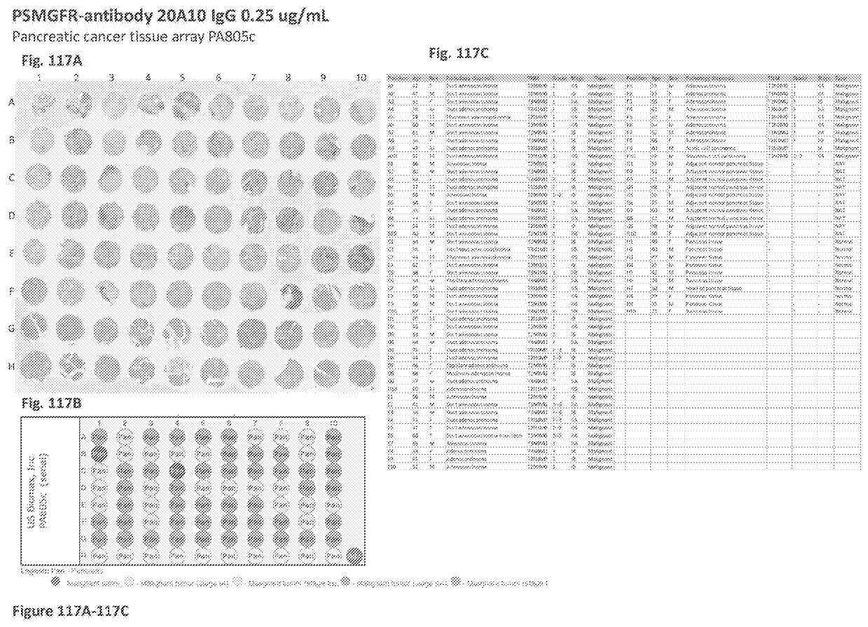

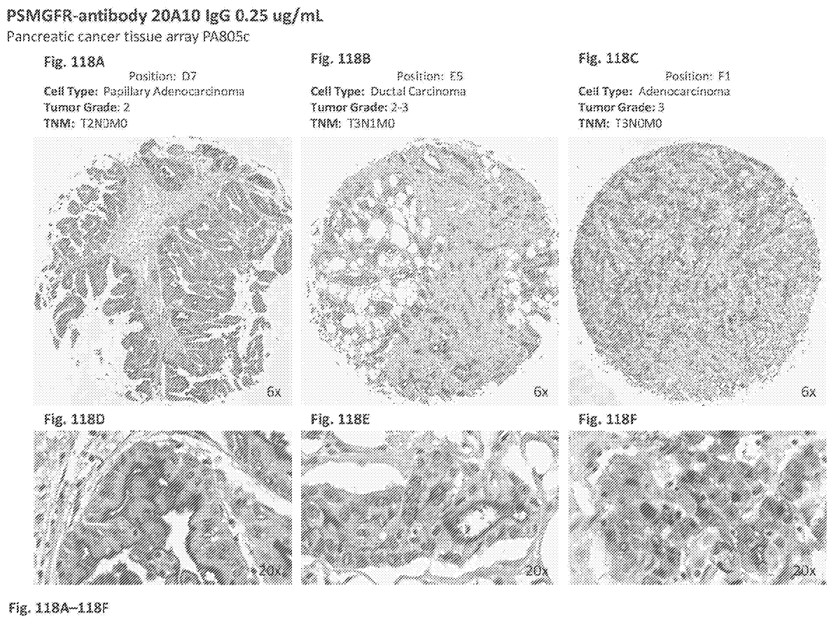

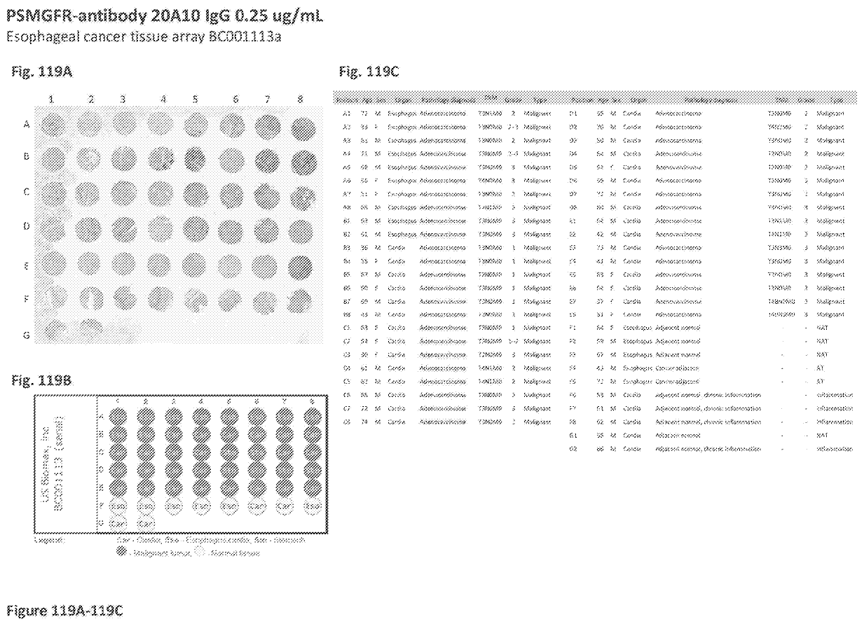

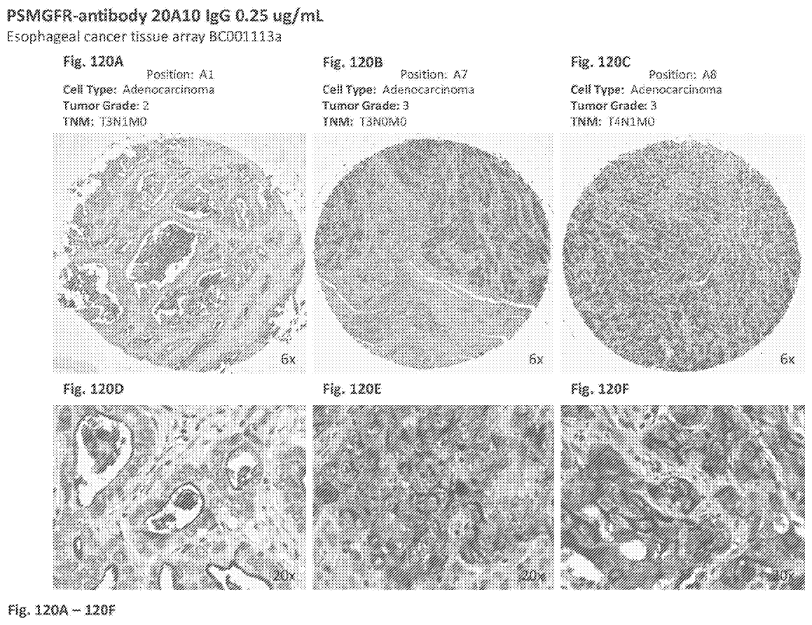

FIG. 113-120 show photographs of tissues studies probed with antibody 20A10. Recall that 20A10 binds to the PSMGFR peptide, binds to the N-10 peptide, but does not bind to the C-10 peptide. Refined epitope mapping shows that like MNC2 and MNE6, the binding of 20A10 depends on amino acids FPFS being present in the PSMGFR peptide. 20A10 binds to the most membrane proximal part of the MUC1* extra cellular domain. An overview of FDA Normal Tissue Array MNO1021 is shown in FIG. 113. FIG. 114A-114X show that there is little to no cross reactivity of 20A10 for normal tissues. We note that MNC2, MNE6 and 20A10 all react with the MUC1* that is expressed on the luminal edge of the terminal breast ducts, luminal edge of the fallopian tubes, luminal edge of about 10% of the distal collecting ducts of normal kidney, and luminal edge of ureter. Because the staining is strictly limited to the luminal edge of a subset of ducts and glands, these antibodies are considered to be safe as therapeutics as the inside of ducts and glands are protected from large entities carried by blood, such as antibodies or CAR T cells. Importantly, MNC2, MNE6 and 20A10 show no staining of critical organs, such as heart, lung and brain. In stark contrast, 20A10, like MNC2 and MNE6, robustly binds to cancerous tissues. 20A10 stains nearly all specimens of the BR1141 breast cancer array (FIGS. 115-116). In addition to robust staining of the breast cancer tissue, the staining is membrane staining, indicating that 20A10 recognizes an extra cellular portion of MUC1*, which is critical for an effective antibody-based anti-cancer therapeutic. 20A10 also showed robust and membranous staining of pancreatic cancer tissues (FIGS. 117-118) and esophageal cancer tissues (FIGS. 119-120). In summary, 20A10 shows great cancer specificity and as an anti-cancer therapeutic offers a large therapeutic window because of the vast difference between staining of normal tissues and cancerous tissues, in terms of the location and intensity of staining.

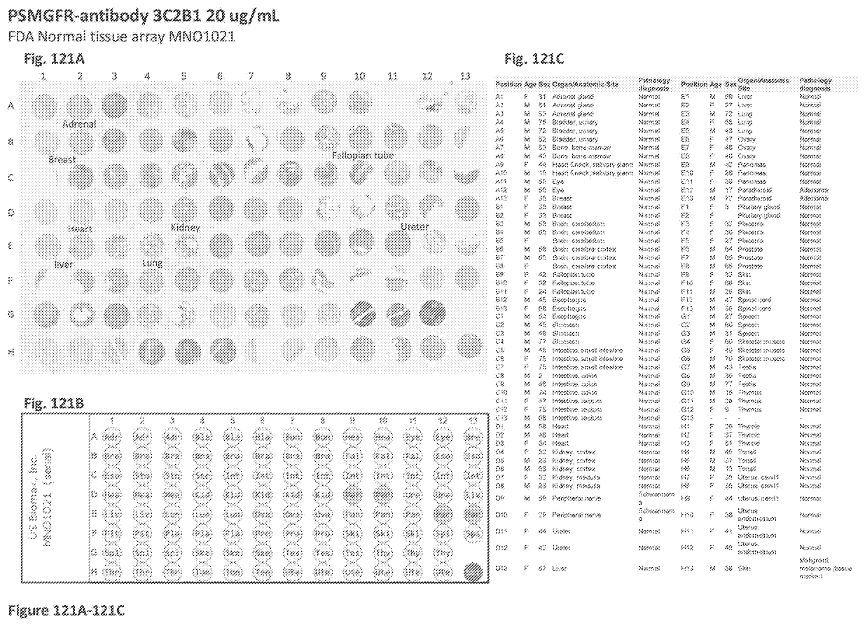

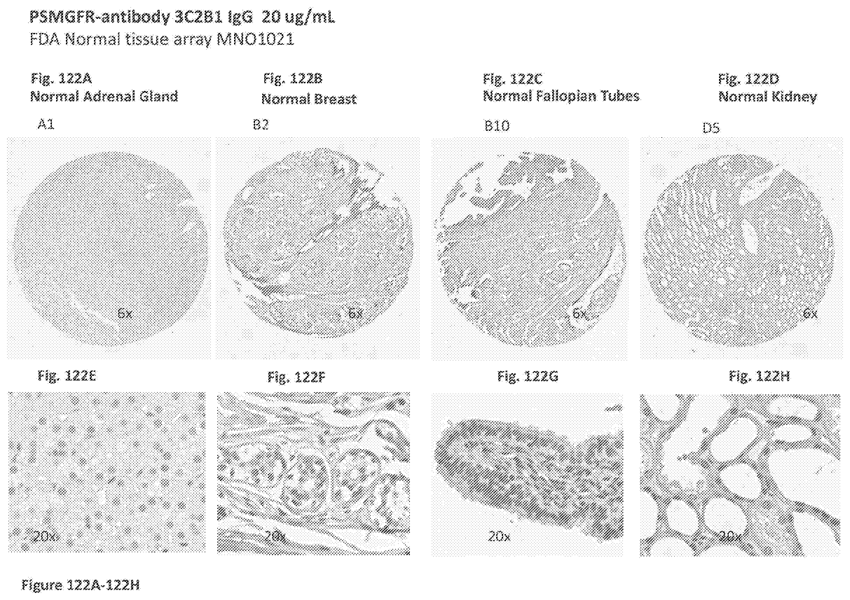

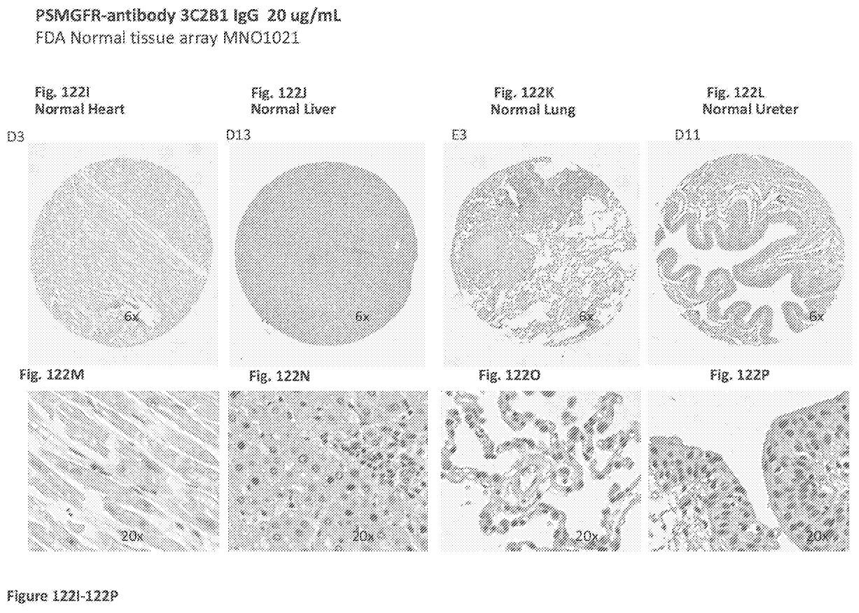

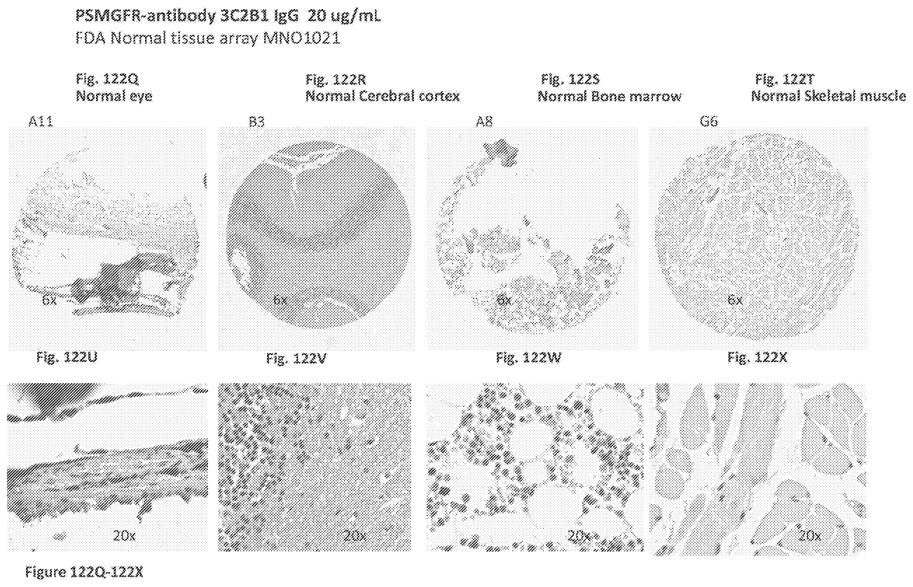

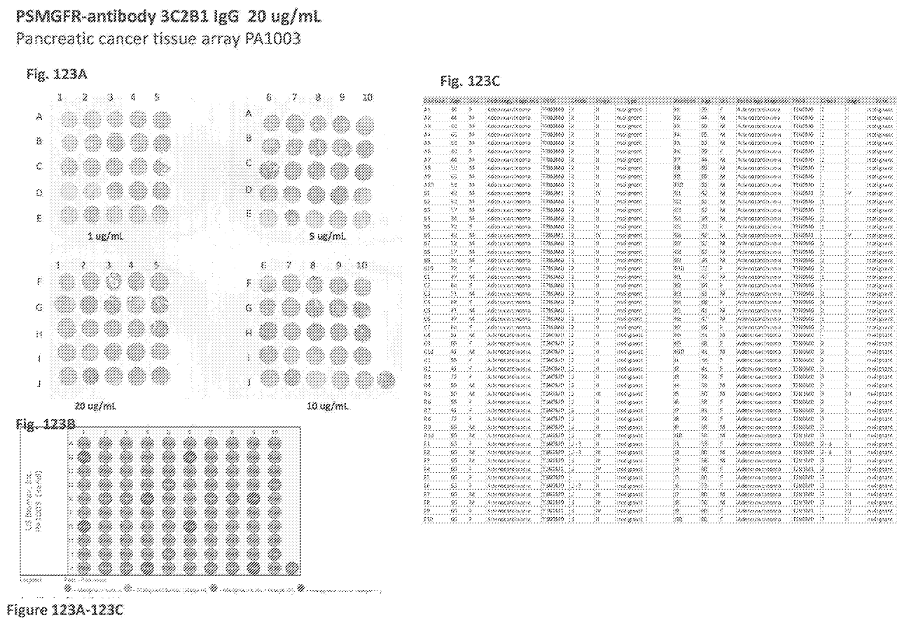

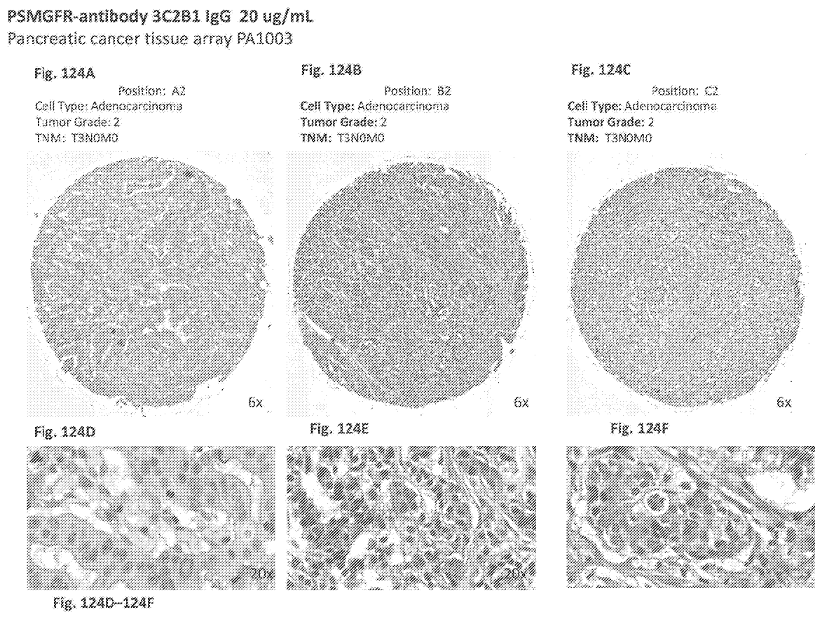

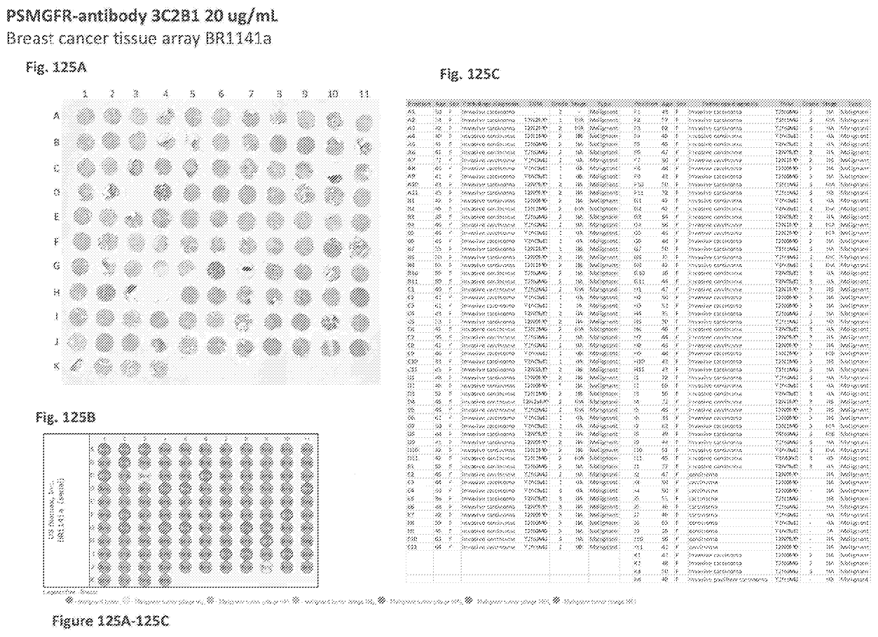

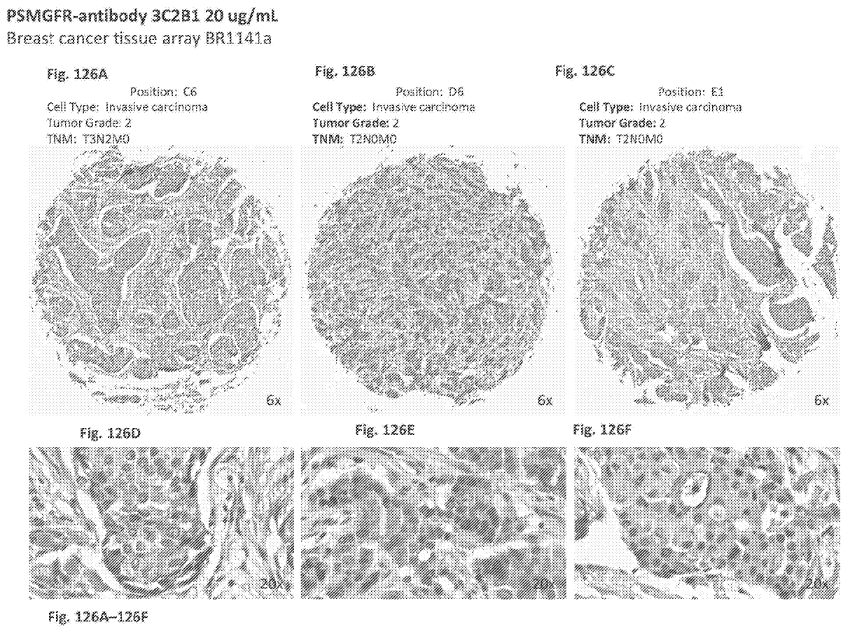

Anti-MUC1* antibody 3C2B1 is an antibody that like MNC2, MNE6 and 20A10, binds to N-10 but not to C-10. More refined epitope mapping shows that like these three other highly cancer-specific antibodies, 3C2B1 requires the FPFS sequence for binding to a MUC1* extra cellular domain peptide. FIG. 121 shows the photograph of the FDA normal array MNO1021. FIGS. 122A-122X shows photographs of specific tissues from FDA normal tissue array MNO1021 stained with the anti-PSMGFR antibody 3C2B1 at 20 ug/mL. As can be seen, there is no binding of 3C2B1 to any critical normal organs. FIG. 123 shows photograph of pancreatic cancer tissue array PA1003 stained with the anti-PSMGFR antibody 3C2B1 at 1-20 ug/mL. FIG. 124 shows photographs of specific tissues from pancreatic cancer tissue array PA1003 stained with the anti-PSMGFR antibody 3C2B1 at 20 ug/mL. FIG. 125 shows photograph of breast cancer tissue array BR1141 stained with the anti-PSMGFR antibody 3C2B1 at 20 ug/mL. FIGS. 126A-126F shows magnified photographs of specific tissues from breast cancer tissue array BR1141 stained with the anti-PSMGFR antibody 3C2B1 at 20 ug/mL. As can be seen in the figure, 3C2B1 robustly stains breast cancer tissues.

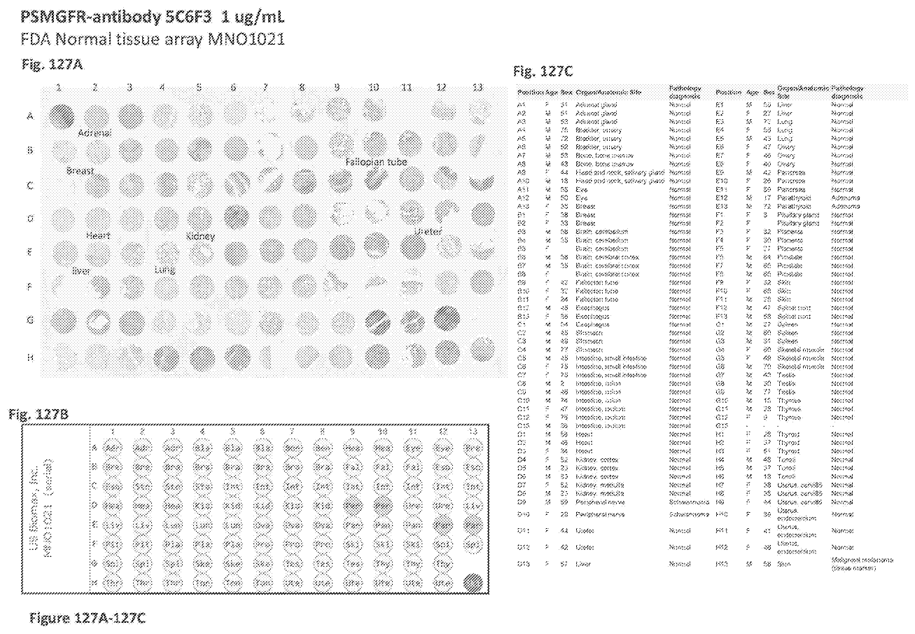

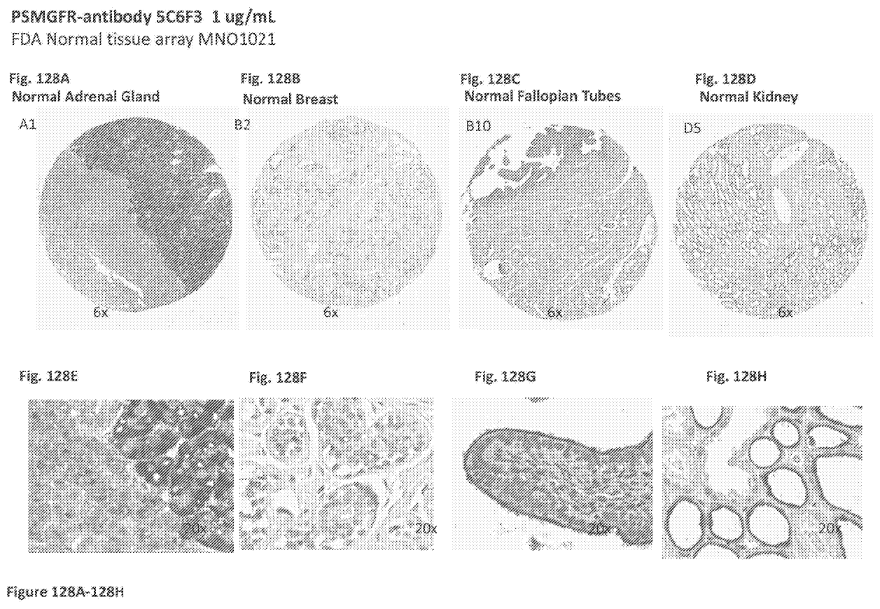

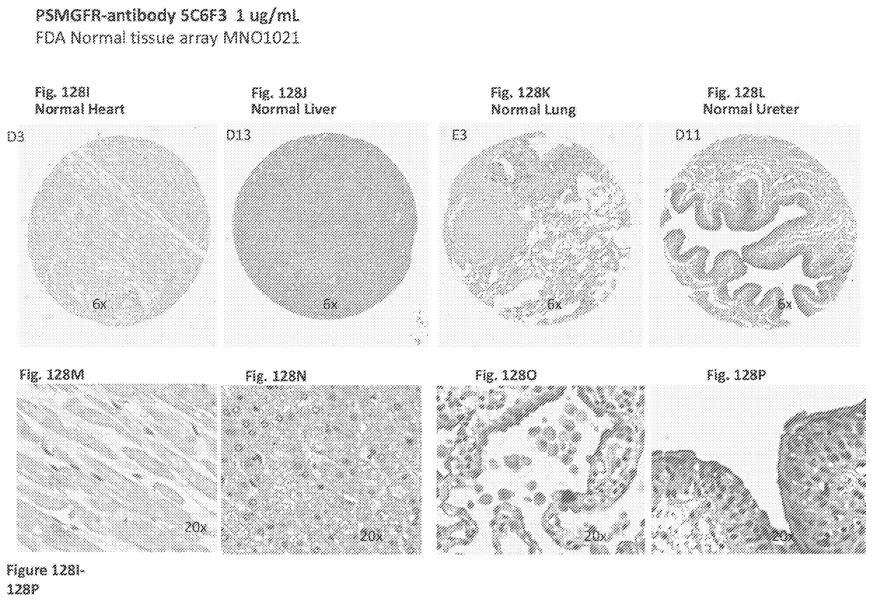

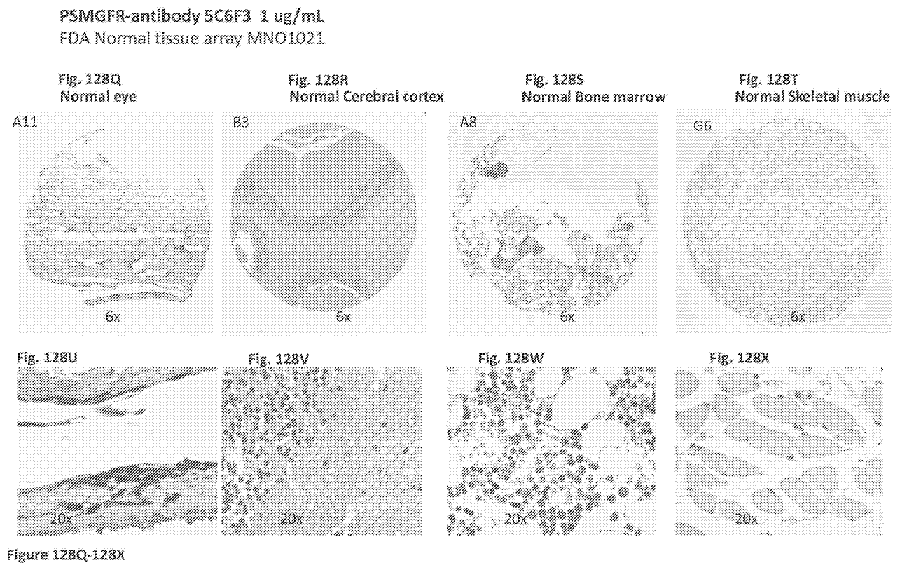

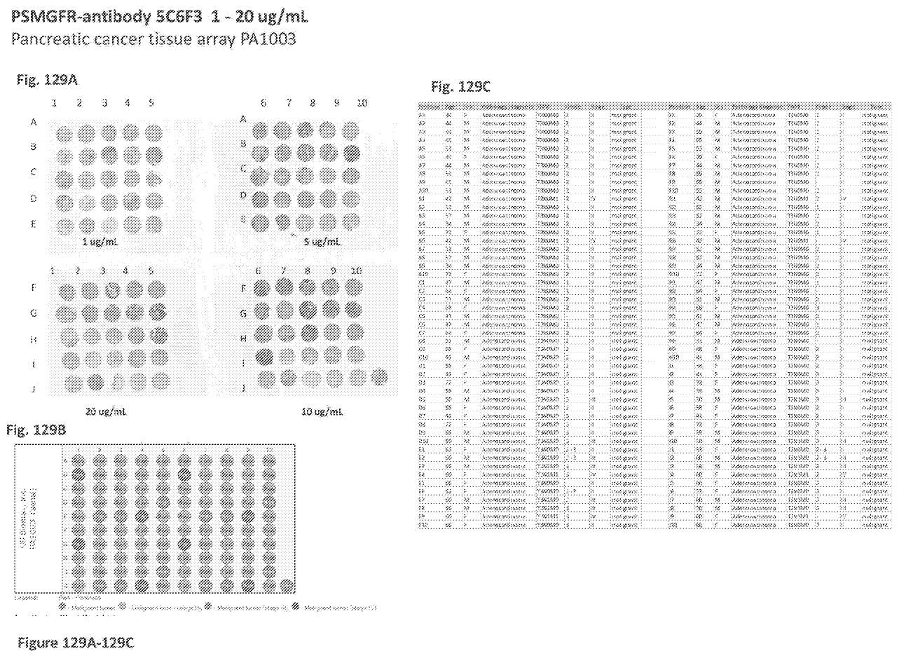

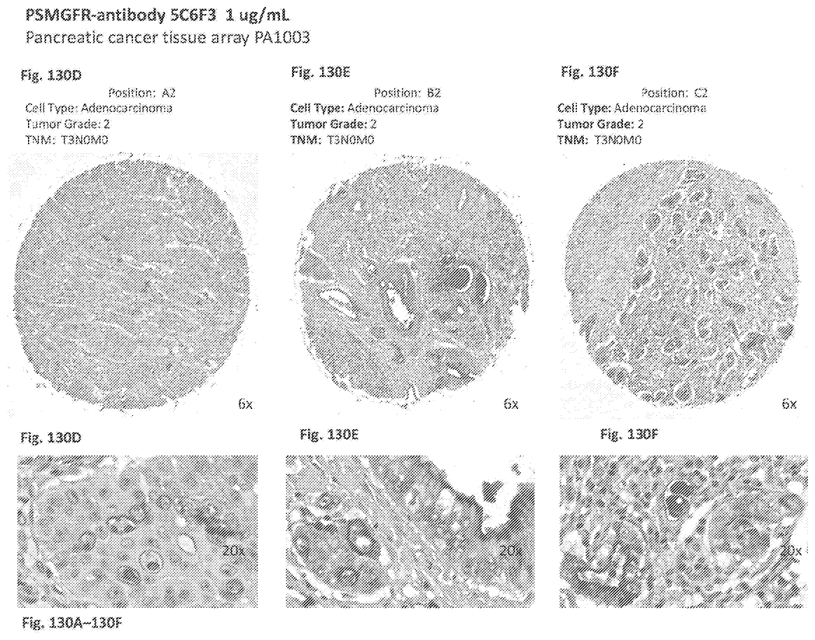

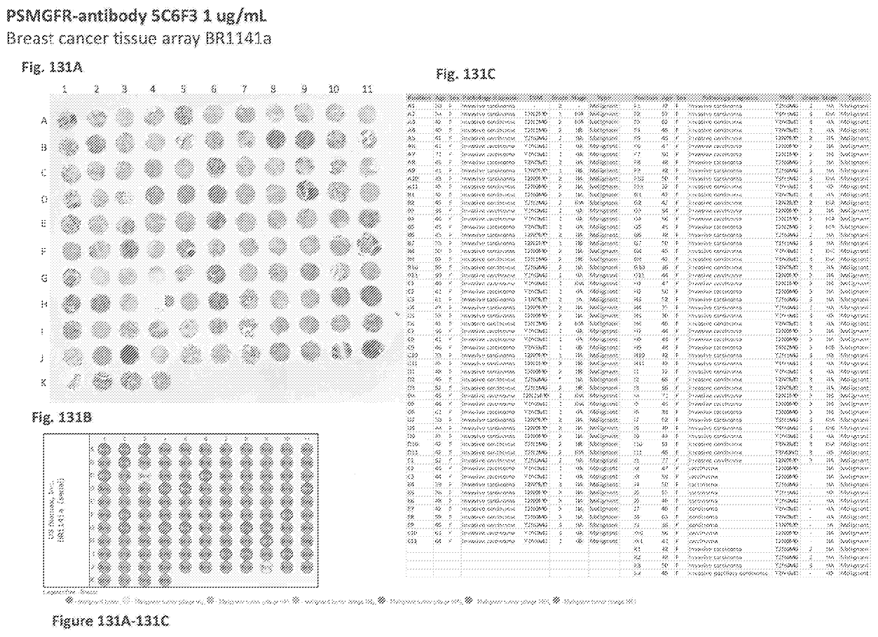

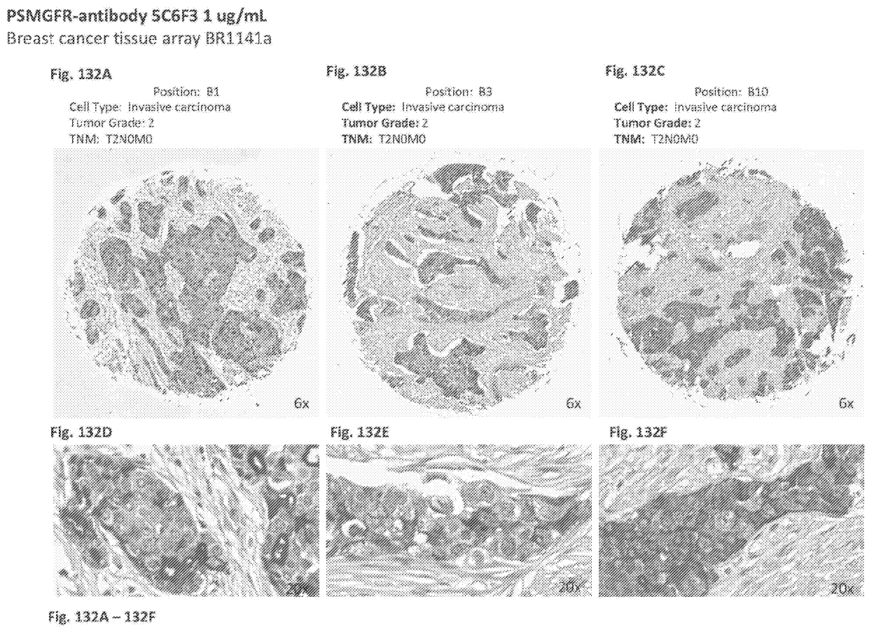

Anti-MUC1* antibody 5C6F3 binds to the N-10 peptide, does bind to the C-10 peptide, although binding is reduced somewhat. Its cognate epitope comprises all or some of the sequence SVSDV (SEQ ID NO:1751). FIG. 127 shows photograph of FDA normal tissue array MNO1021 stained with the anti-PSMGFR antibody 5C6F3 at 1 ug/mL. FIG. 128 shows photographs of specific tissues from FDA normal tissue array MNO1021 stained with the anti-PSMGFR antibody 5C6F3 at 1 ug/mL. FIG. 129 shows photograph of pancreatic cancer tissue array PA1003 stained with the anti-PSMGFR antibody 5C6F3 at 1-20 ug/mL. FIG. 130 shows photographs of specific tissues from pancreatic cancer tissue array PA1003 stained with the anti-PSMGFR antibody 5C6F3 at 1 ug/mL. FIG. 131 shows photograph of breast cancer tissue array BR1141 stained with the anti-PSMGFR antibody 5C6F3 at 1 ug/mL. FIG. 132 shows photographs of specific tissues from breast cancer tissue array BR1141 stained with the anti-PSMGFR antibody 5C6F3 at 1 ug/mL. As can be seen in the FIG. 5C6F3 is a high affinity antibody that has great cancer-specificity and with the exception of adrenal, which may be an artefact of that tissue, did not show binding to normal tissues.

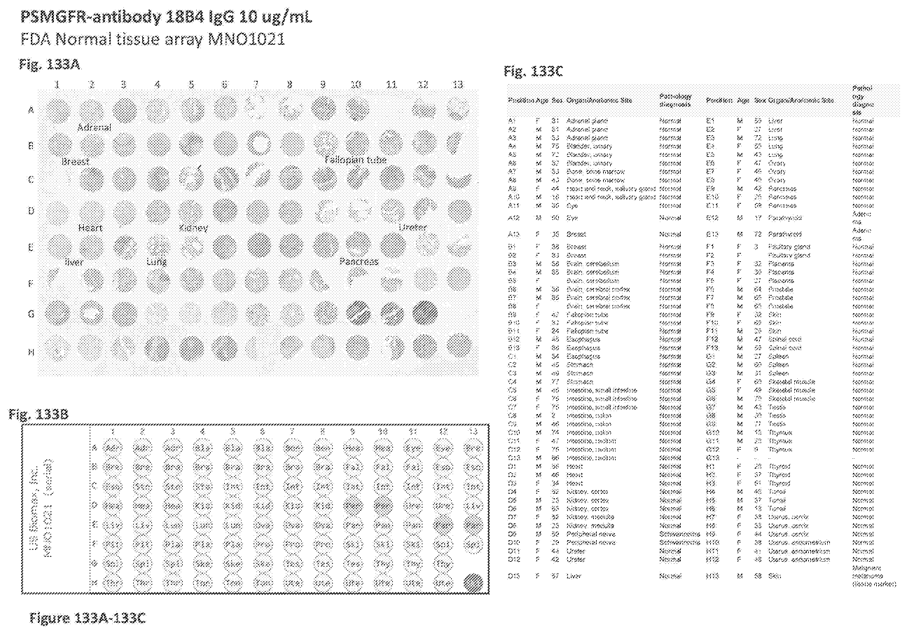

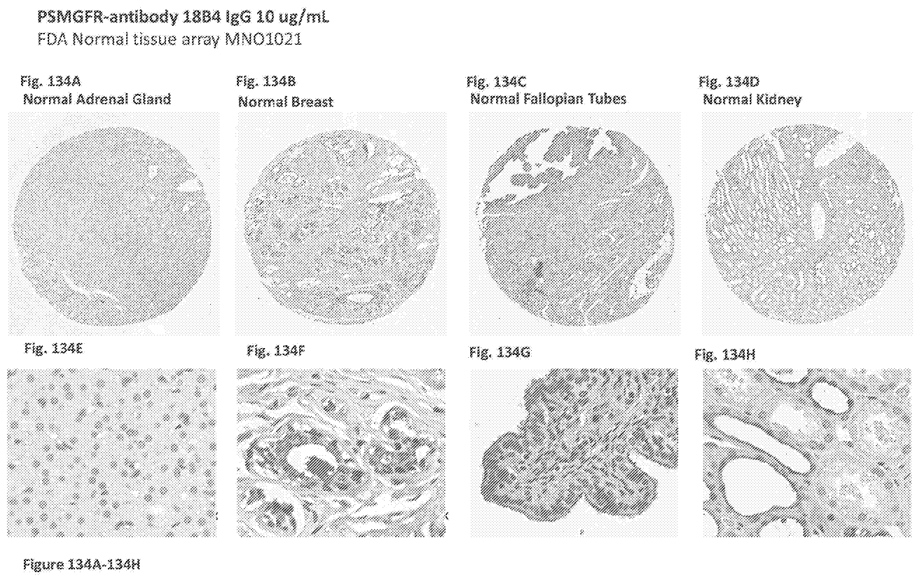

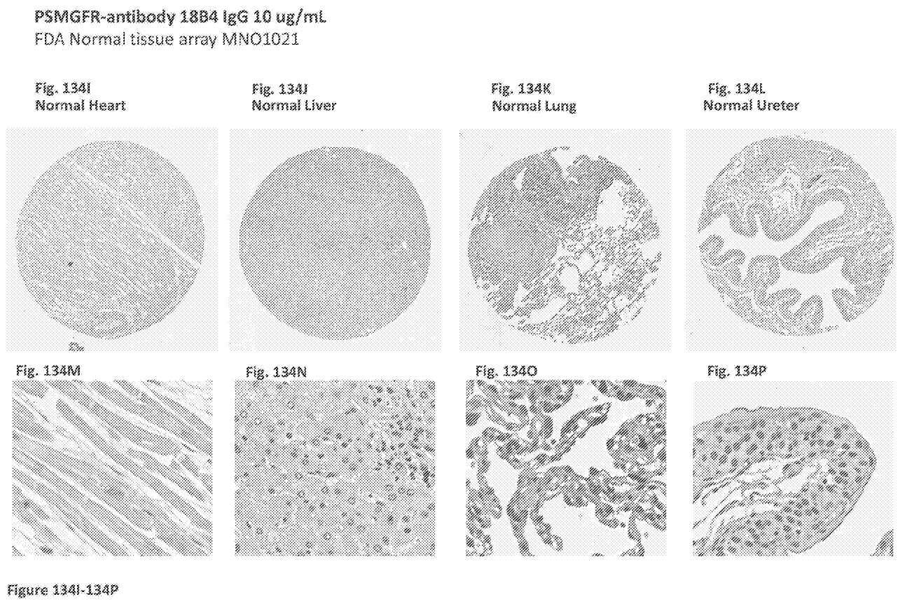

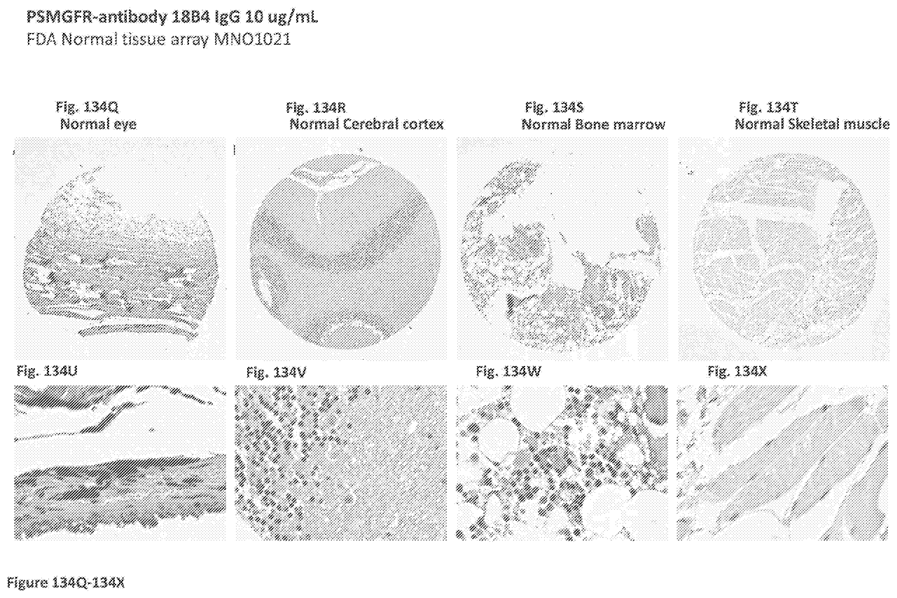

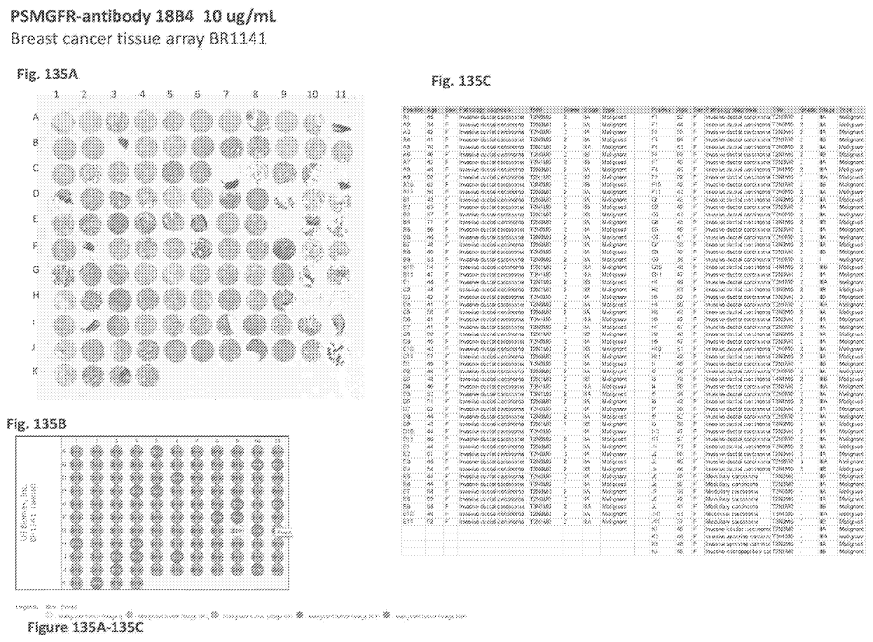

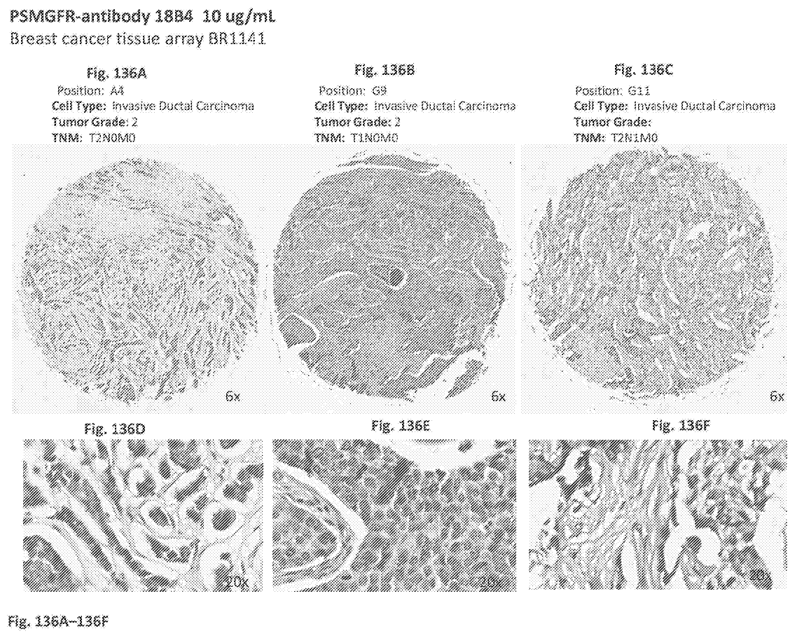

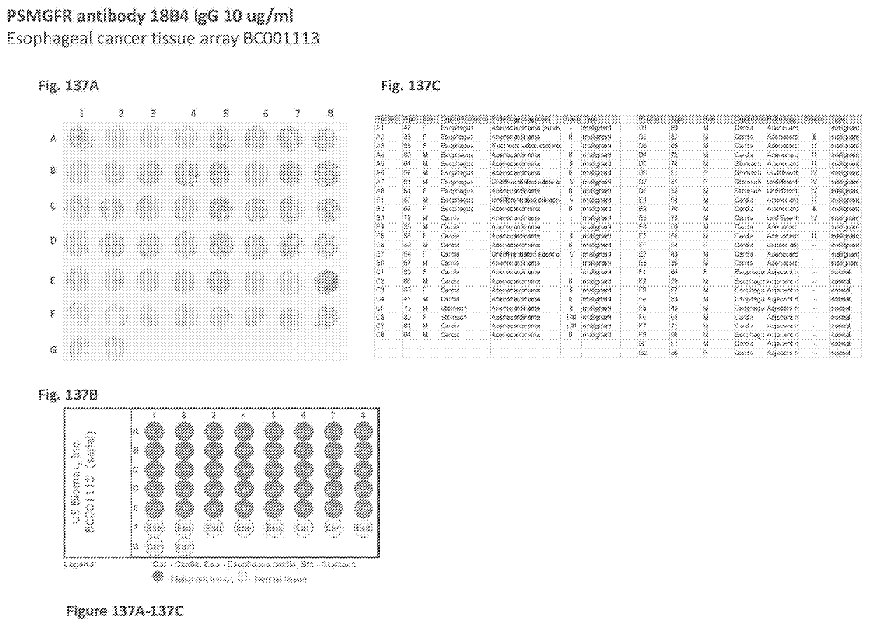

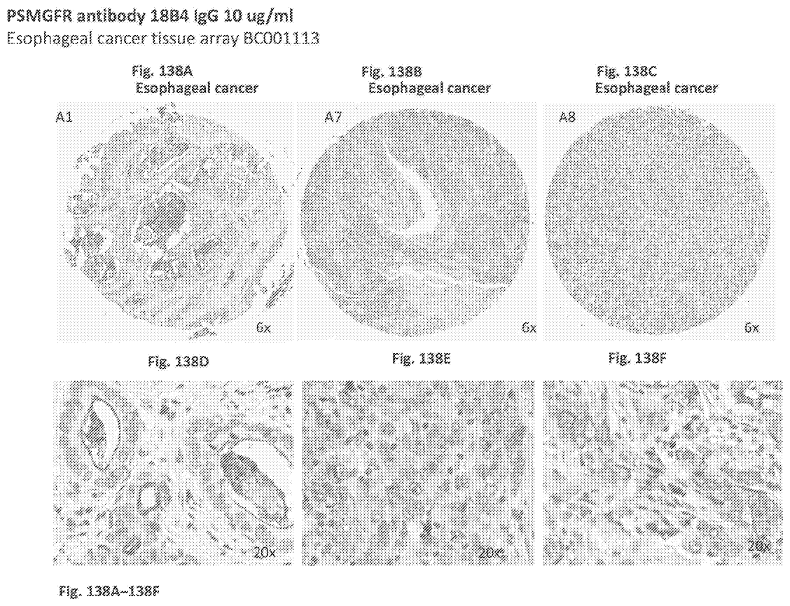

In contrast to 20A10, which binds to the most membrane proximal part of the MUC1* extra cellular domain, 18B4 binds within the GTINVHDVET sequence, which is the most distal part of the PSMGFR sequence. Unlike antibodies MNC2, MNE6 or 20A10, 18B4 cannot bind to the N-10 peptide but does bind to the C-10 peptide. FIG. 133-134 show the binding of antibody 18B4 to normal tissues. In contrast to 20A10, antibody 18B4 shows strong binding to a wide range of normal tissues (FIG. 134), including lung (FIG. 134K). FIG. 135-138 show 18B4 staining of breast cancer tissues and esophageal cancer tissues. Because of the strong binding of 18B4 to normal tissues, there is less cancer specificity to this antibody.

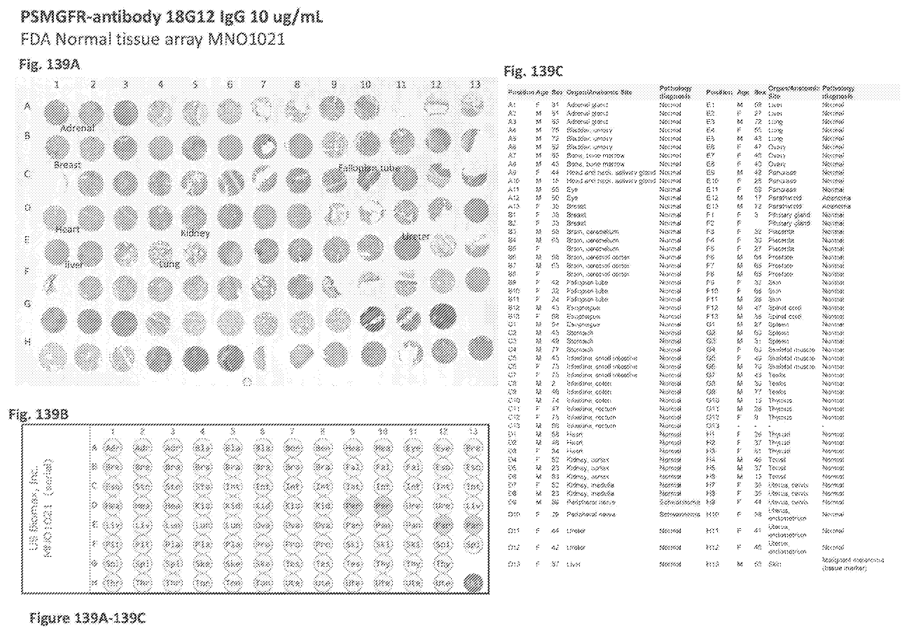

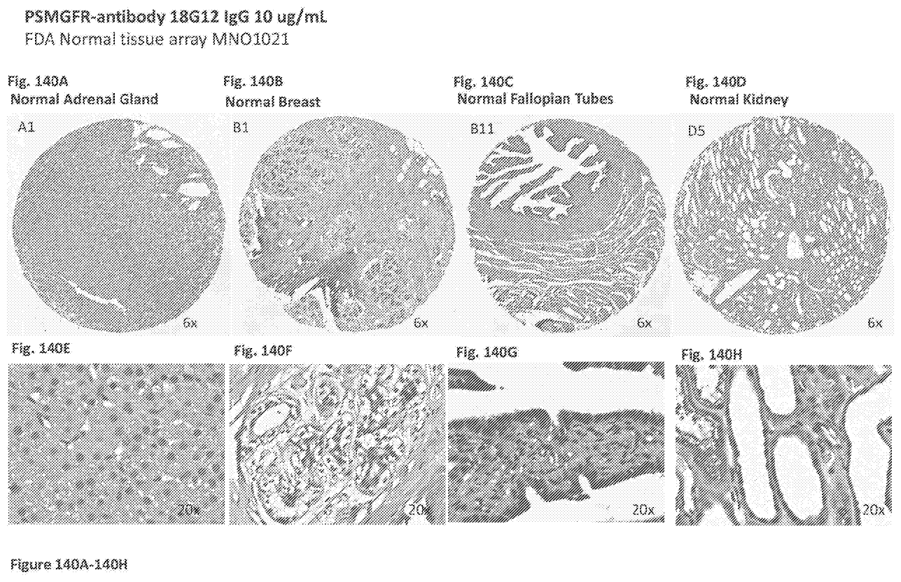

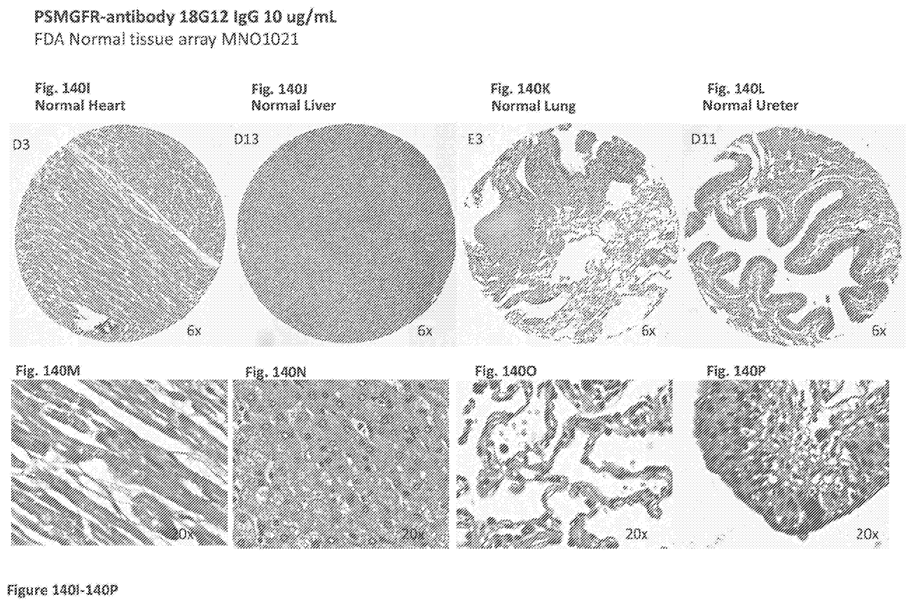

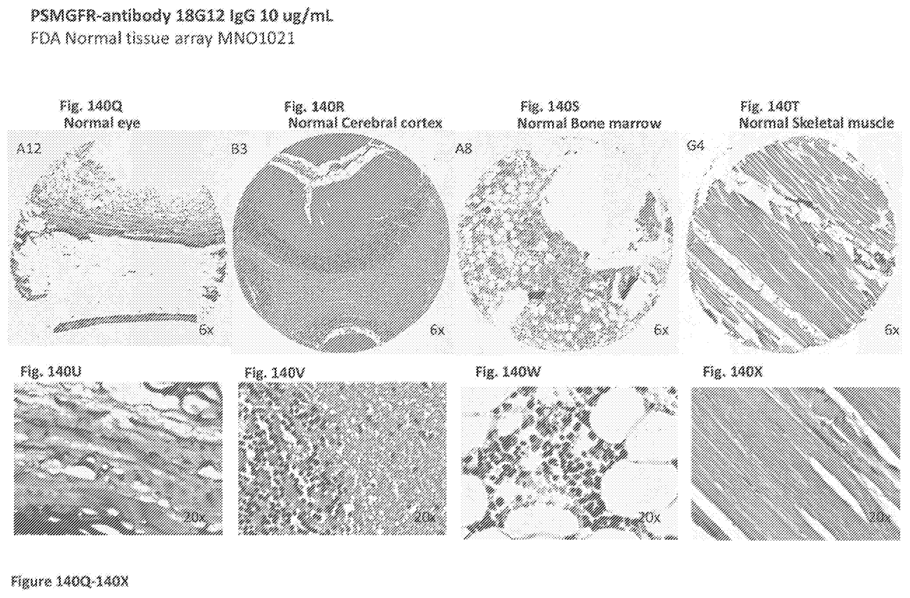

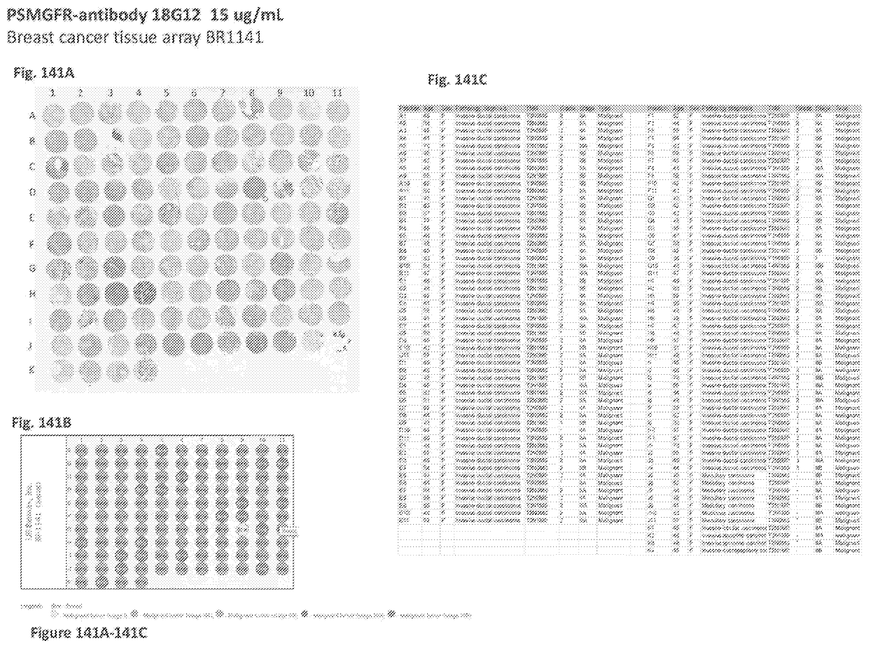

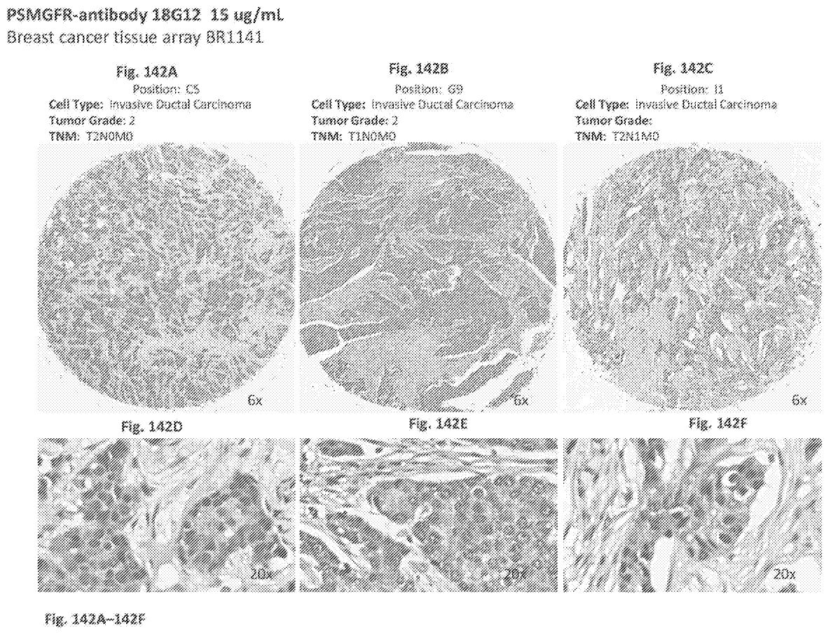

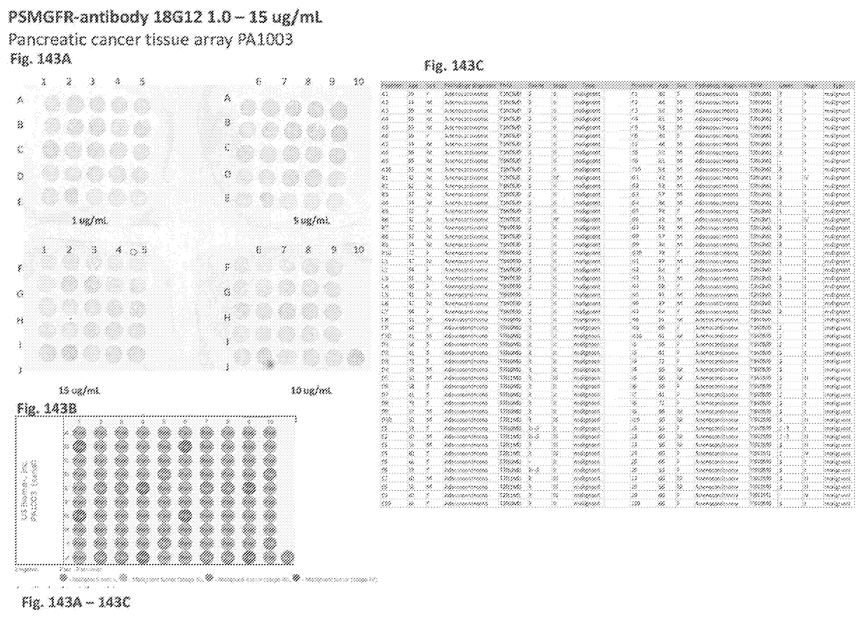

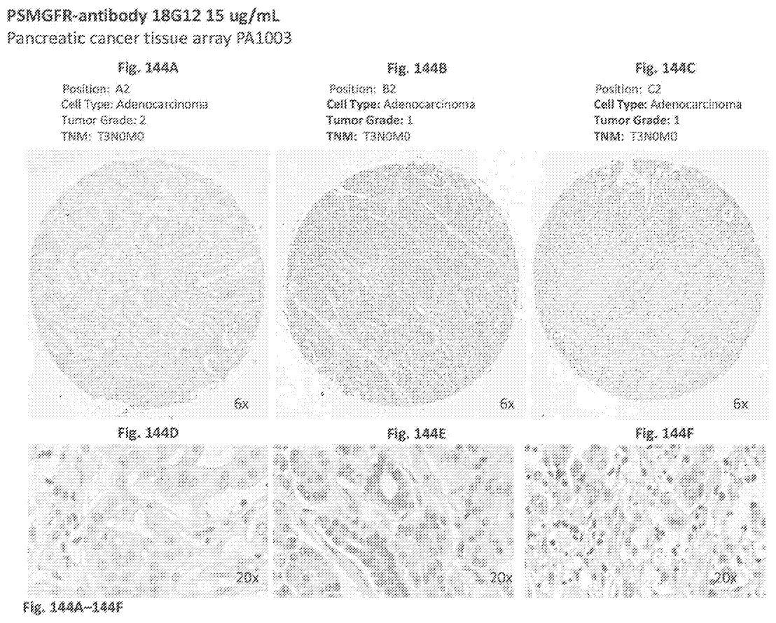

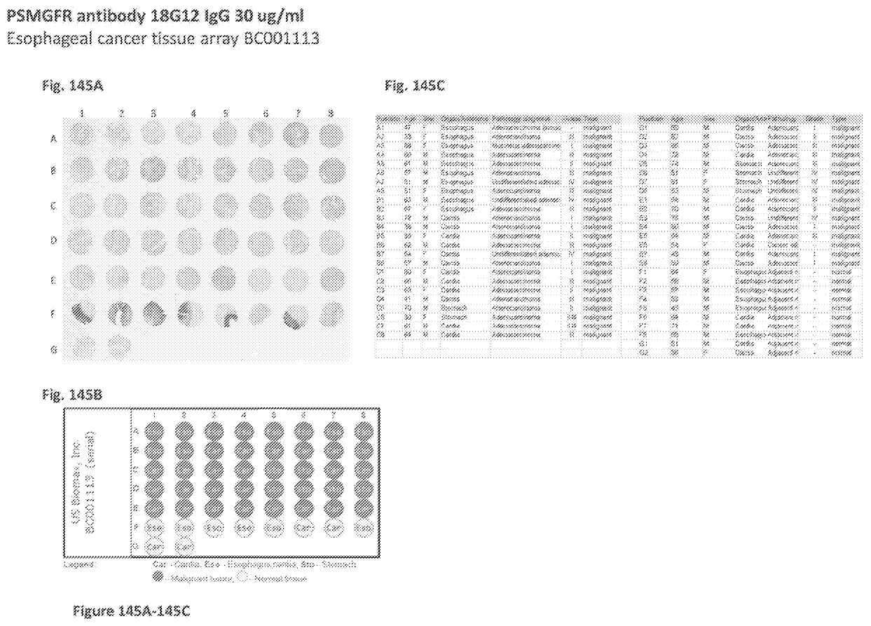

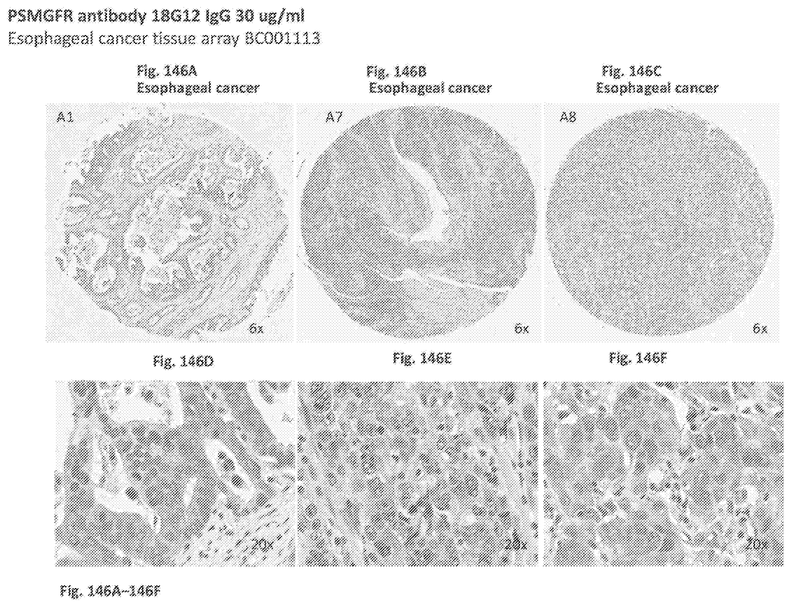

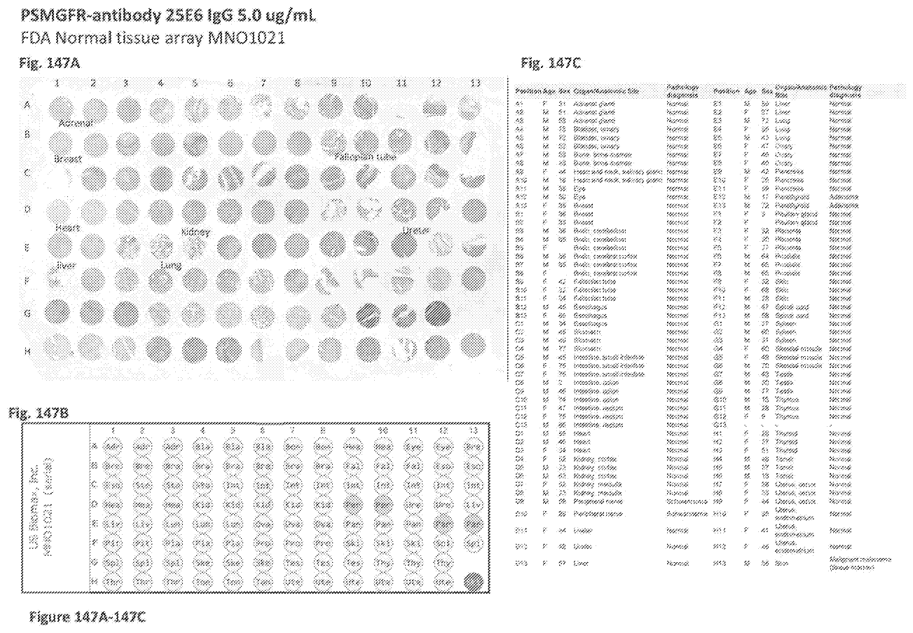

FIG. 139-144 show the binding of PSMGFR antibody 18G12 to normal tissues, breast cancer tissues and esophageal cancer tissues. 18G12 is able to bind to the N-10 peptide, but is also able to bind to the C-10 peptide. 18G12 binds to the ASRYNLT epitope within the PSMGFR peptide. Antibody 18G12 binds to the luminal edge of many of the collecting ducts of normal kidney (FIG. 140D), binds to normal heart muscle (FIG. 140I) as well as to normal skeletal muscle (FIG. 140X). However, there is a clear cancer specificity in that 18G12 binds much more strongly to cancerous tissues than to the few normal tissues. In addition, 18G12 stains the entire cancerous tissues rather than just a luminal edge here or there. FIG. 141-146 show 18G12 staining of breast cancer tissues, pancreatic cancer tissues and esophageal cancerous tissues. The contrast between the staining of the normal tissues and the cancer tissues clearly demonstrates cancer specificity.

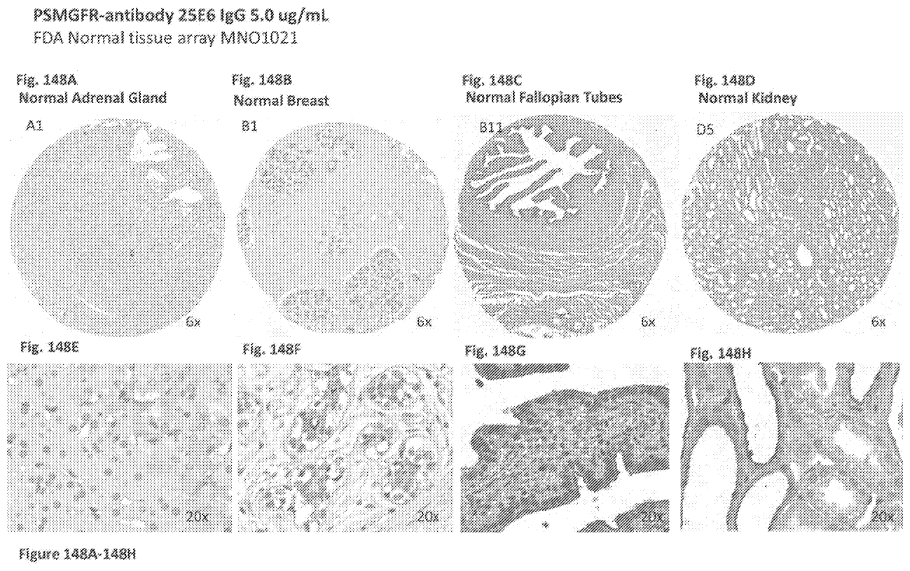

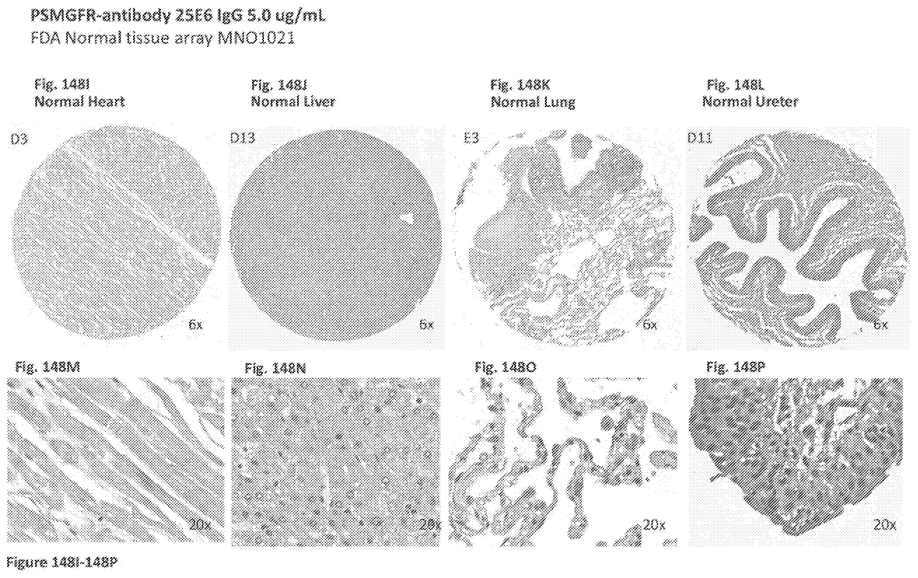

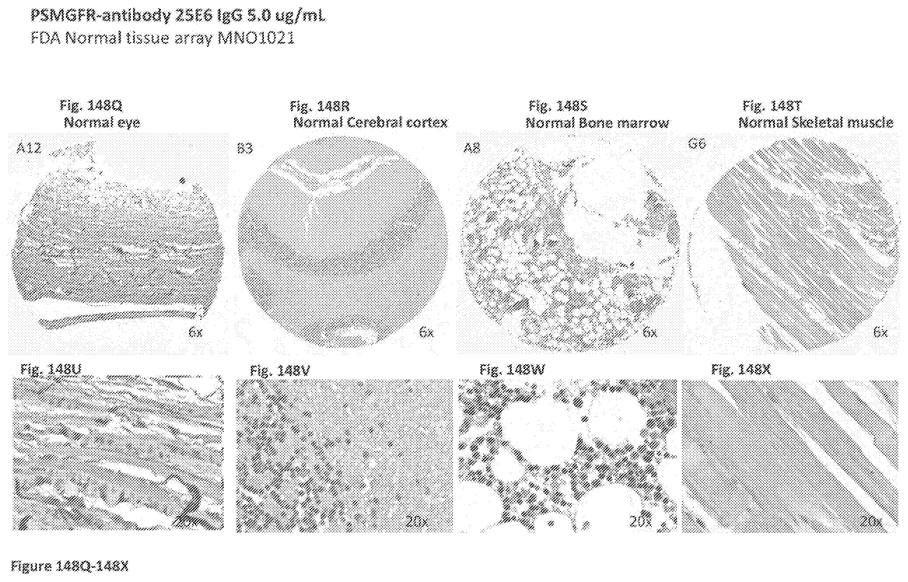

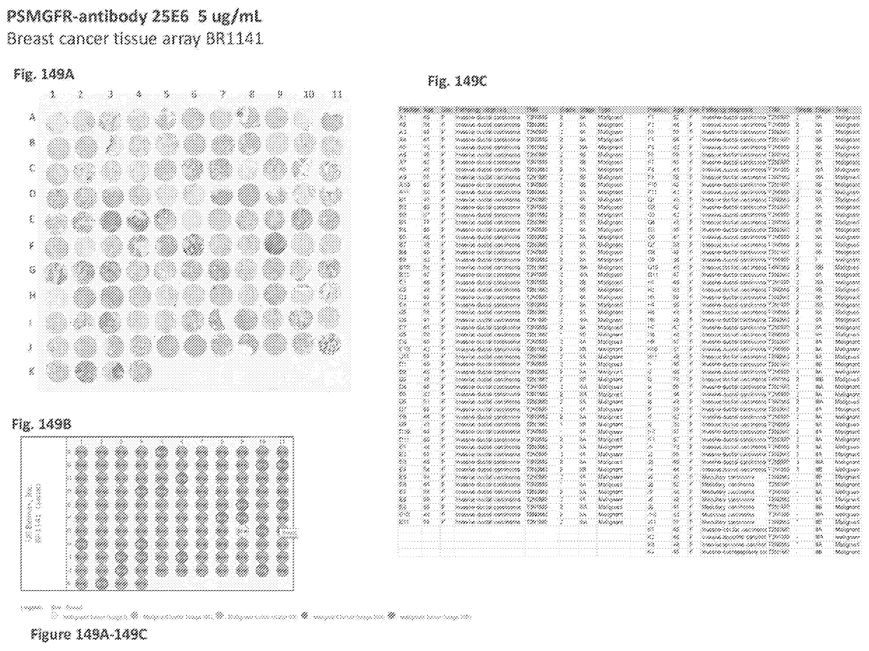

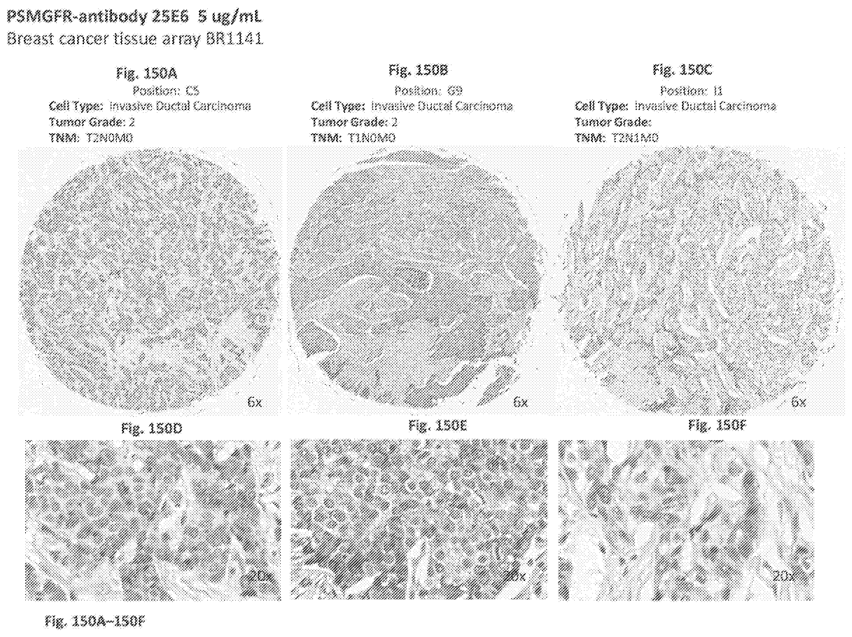

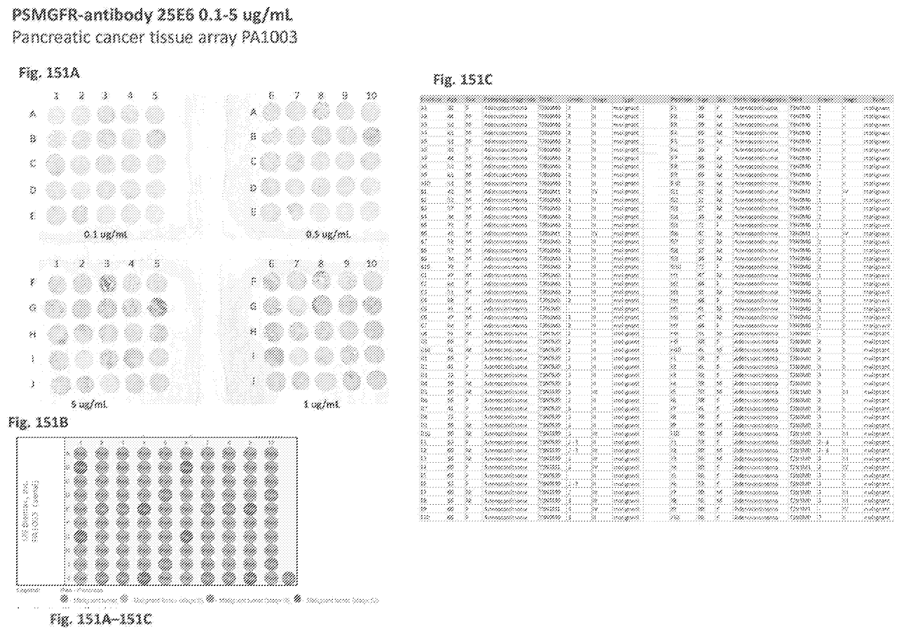

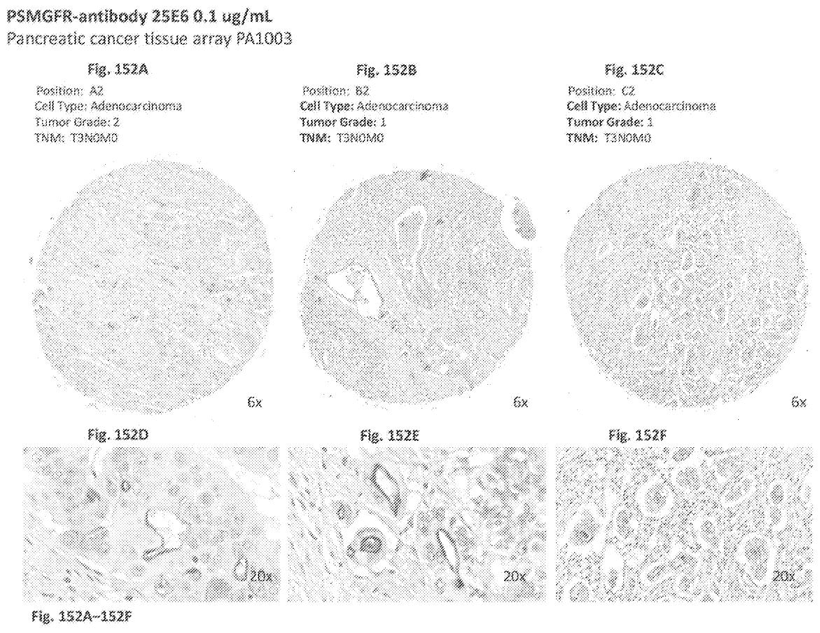

FIG. 147-148 show the binding of PSMGFR antibody 25E6 to normal tissues. 25E6 is able to bind to the N-10 peptide, but is also able to bind to the C-10 peptide. 25E6 binds to the ASRYNLT epitope within the PSMGFR peptide. Like MNC2, MNE6 and 20A10, antibody 25E6 binds to the luminal edge of terminal breast ducts, luminal edge of fallopian tubes, to the luminal edge of a subset of the distal collecting ducts of normal kidney and to the luminal edge of ureter. Unlike MNC2, MNE6 and 20A10, 25E6 binds, albeit very weakly, to normal heart muscle (FIG. 148I) as well as to normal skeletal muscle (FIG. 148X). However, there is a clear cancer specificity in that 25E6 binds much more strongly to cancerous tissues than to the few normal tissues. In addition, 25E6 stains the entire cancerous tissues rather than just a luminal edge here or there. FIG. 149-152 show 25E6 staining of breast cancer tissues and pancreatic cancerous tissues. The contrast between the staining of the normal tissues and the cancer tissues clearly demonstrates cancer specificity.

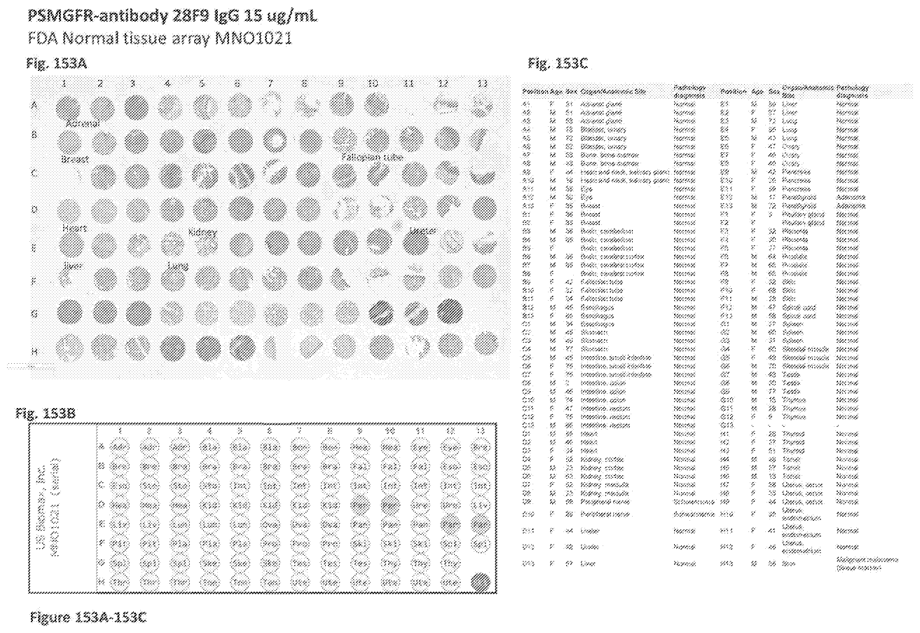

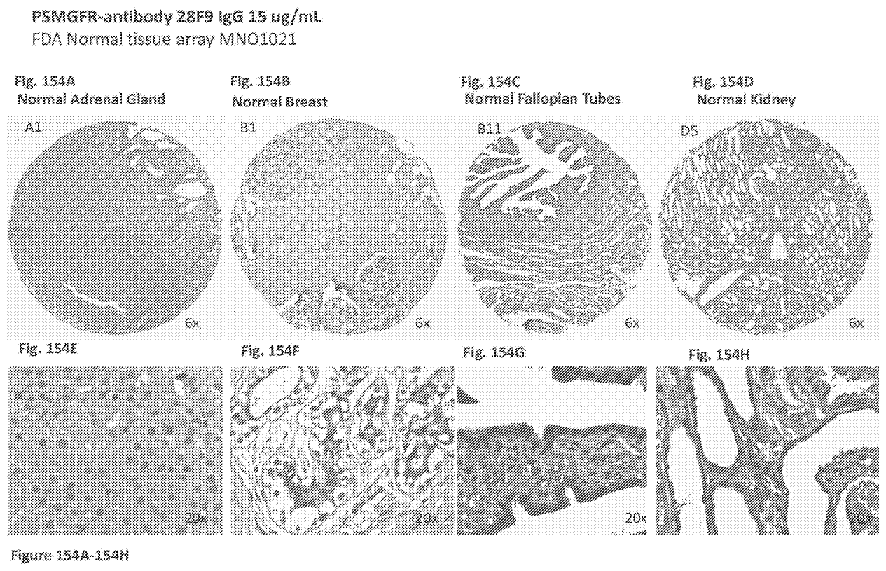

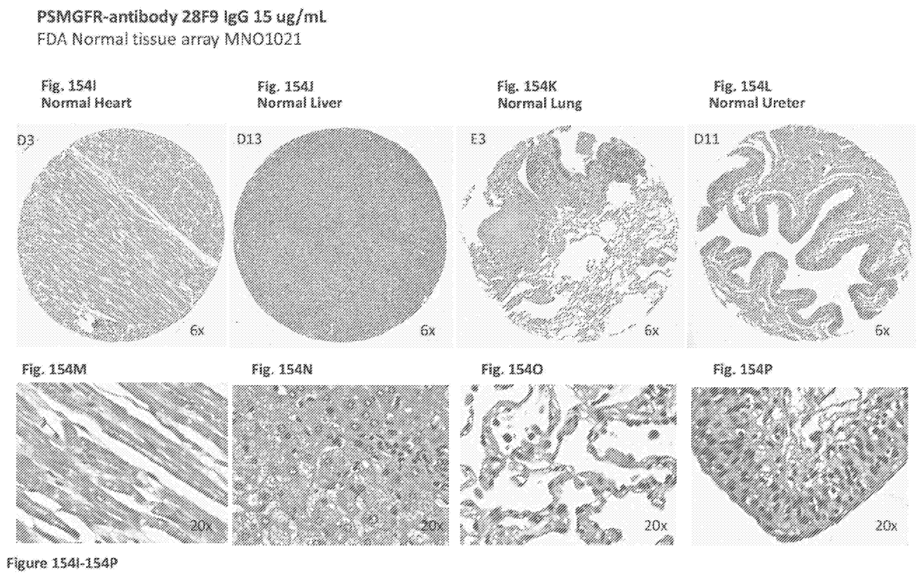

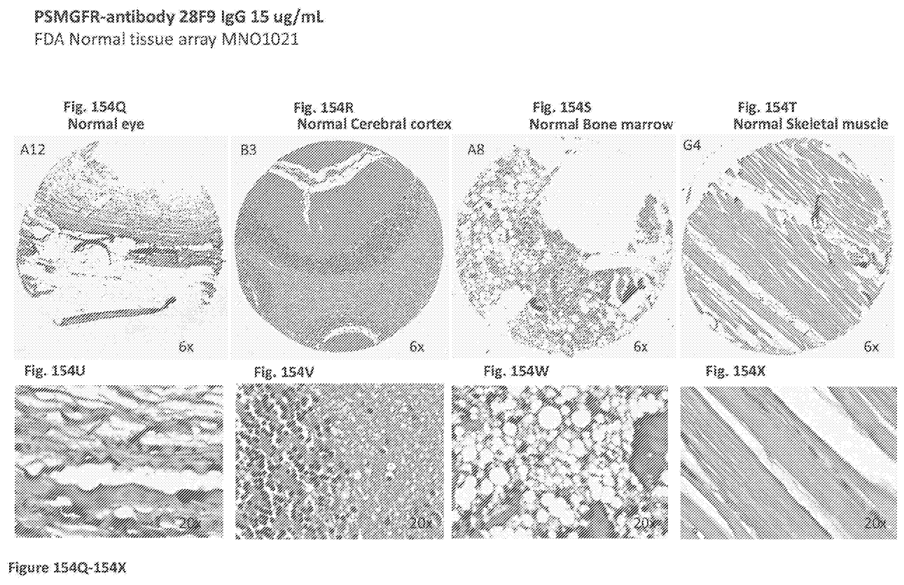

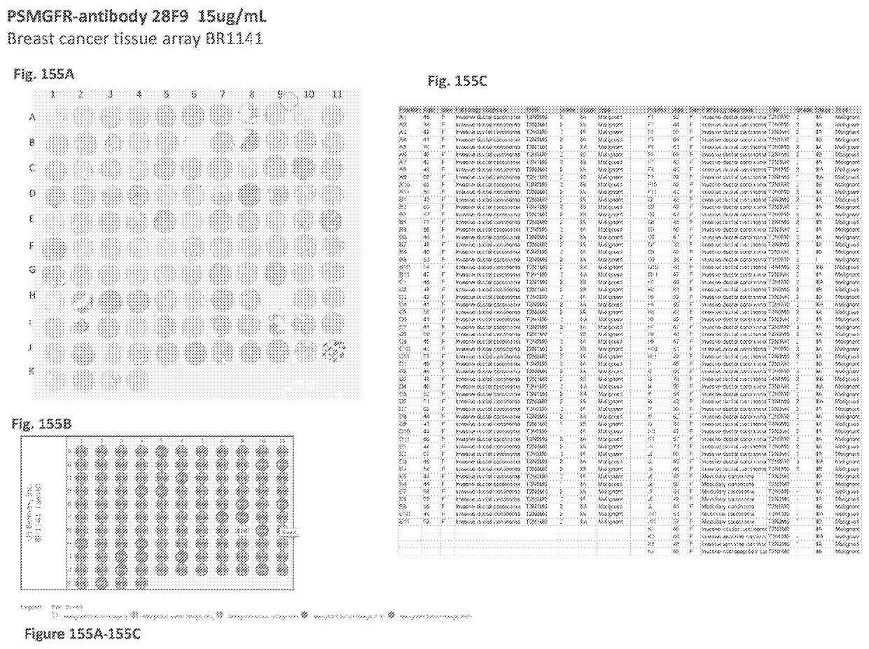

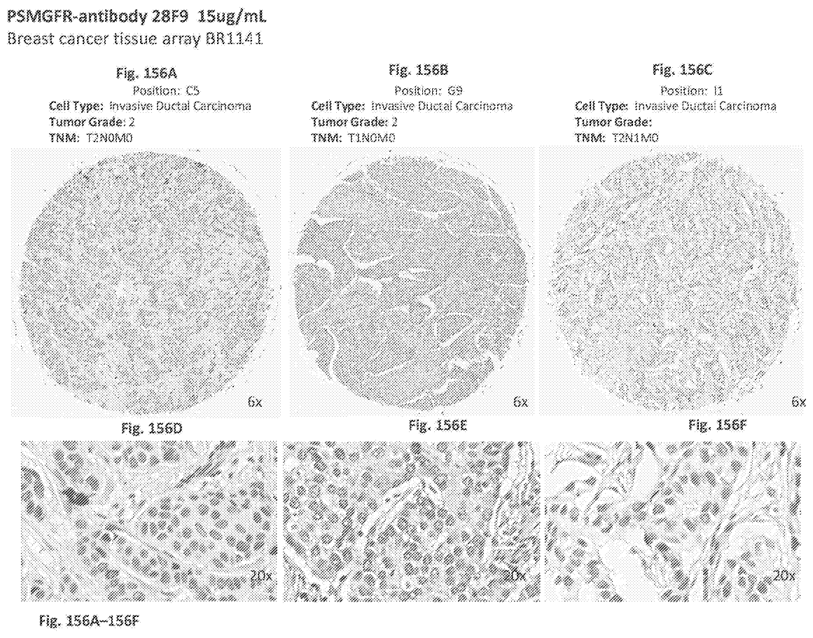

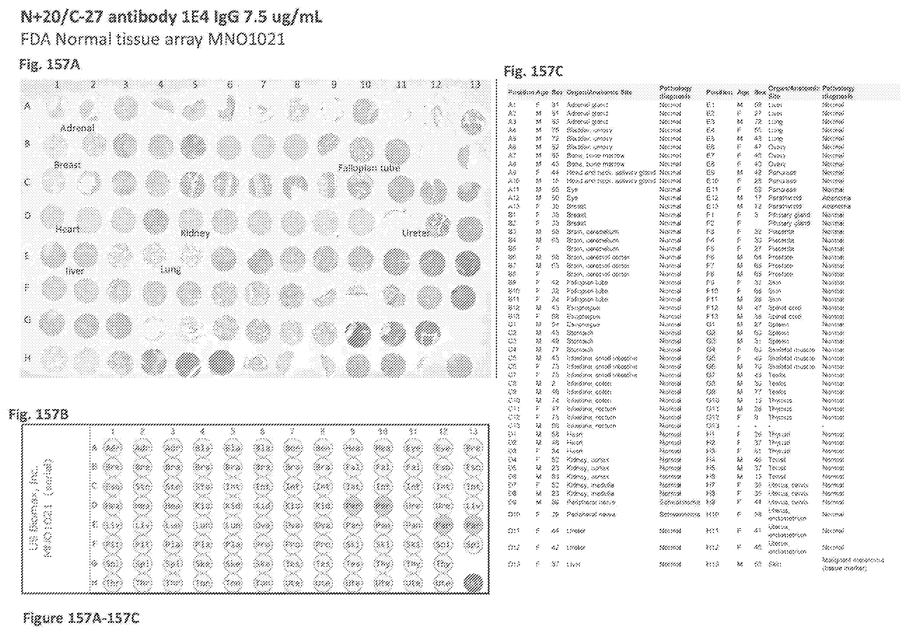

FIG. 153-156 show the binding of PSMGFR antibody 28F9 to normal tissues and breast cancer tissues. 28F9 is able to bind to the N-10 peptide, but is also able to bind to the C-10 peptide. 28F9 binds to the ASRYNLT epitope within the PSMGFR peptide. Like MNC2, MNE6 and 20A10, antibody 25E6 binds to the luminal edge of terminal breast ducts, luminal edge of fallopian tubes, to the luminal edge of a subset of the distal collecting ducts of normal kidney and to the luminal edge of ureter. FIG. 155-156 show 28F9 staining of breast cancer tissues.

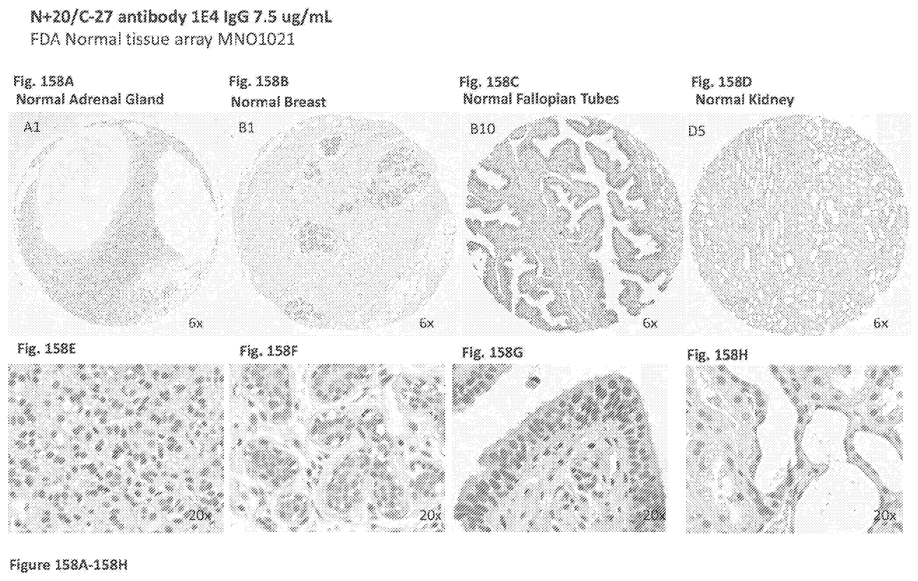

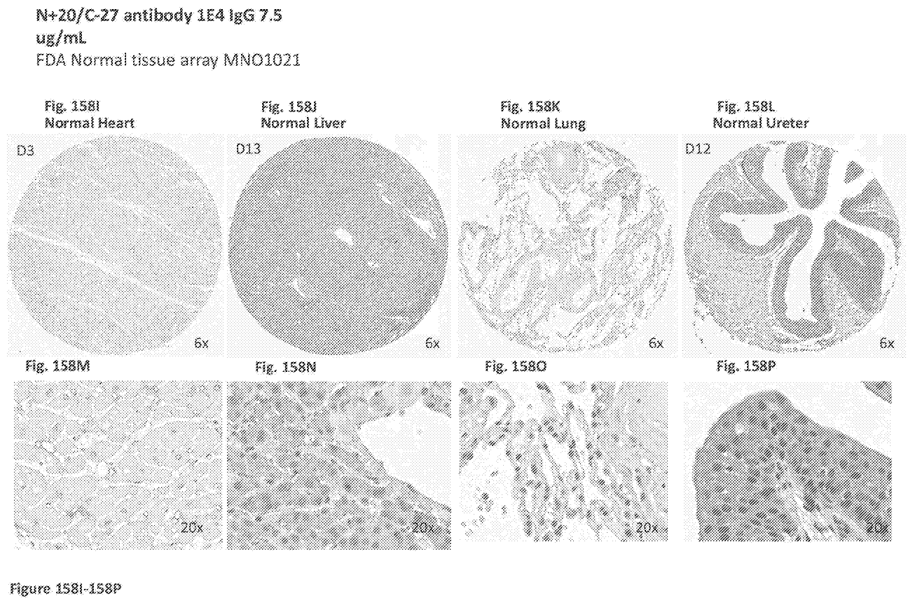

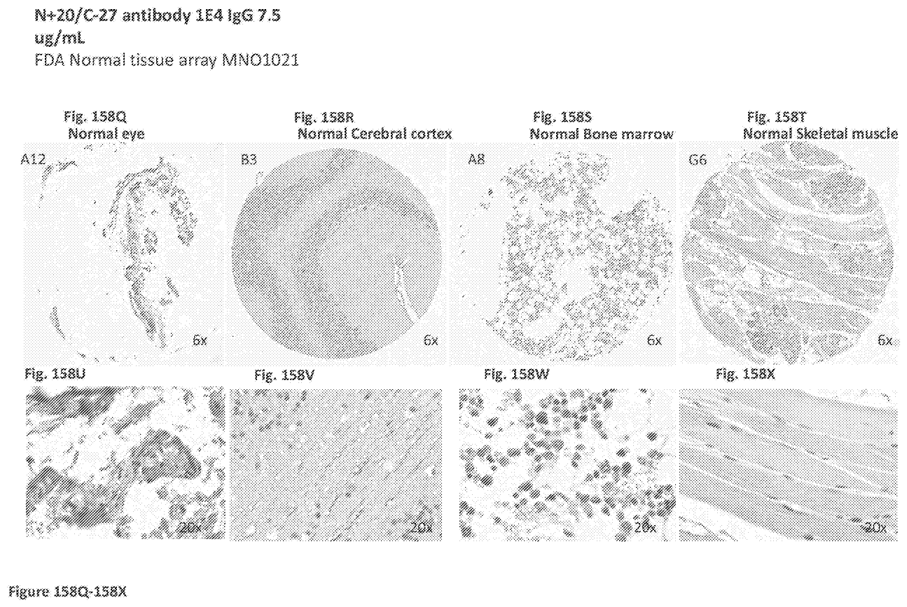

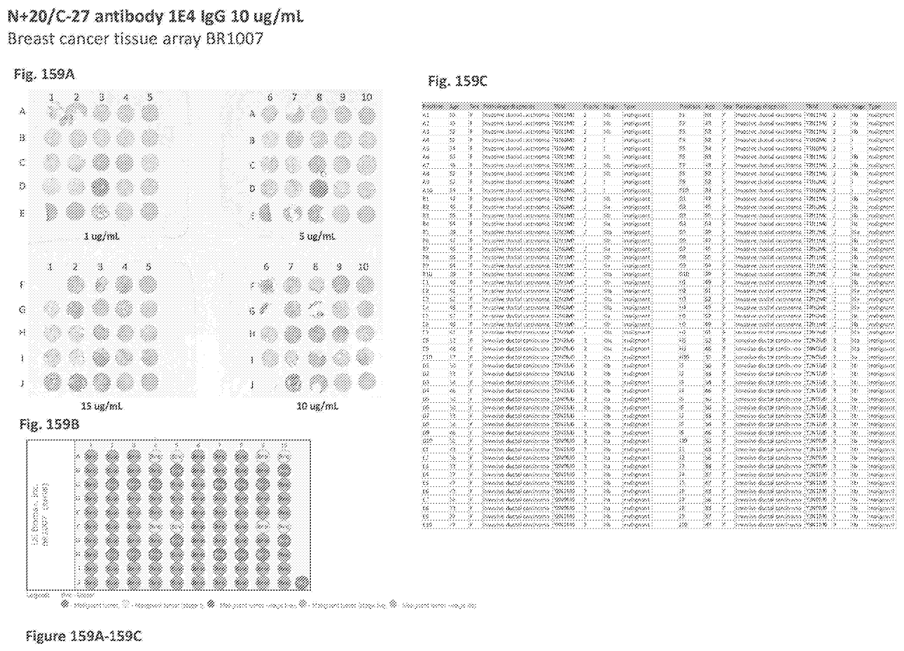

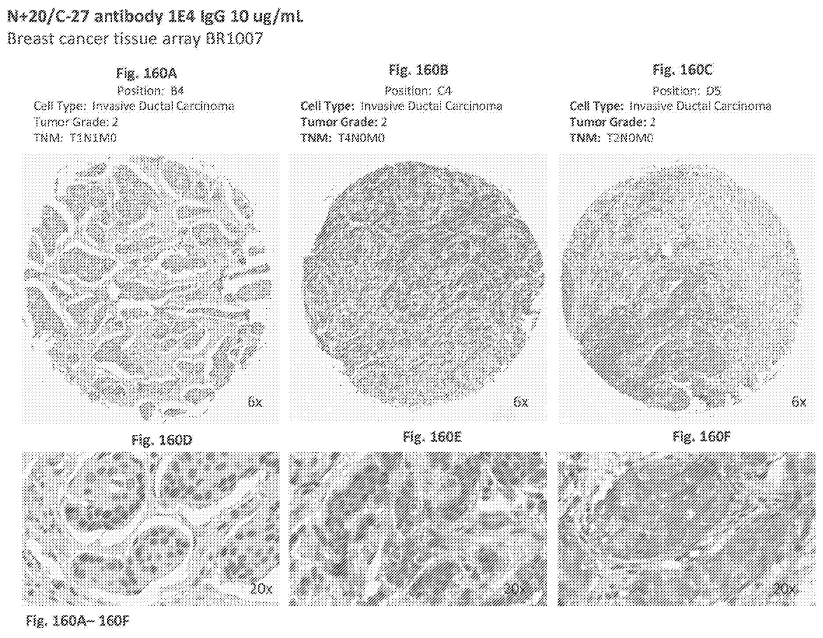

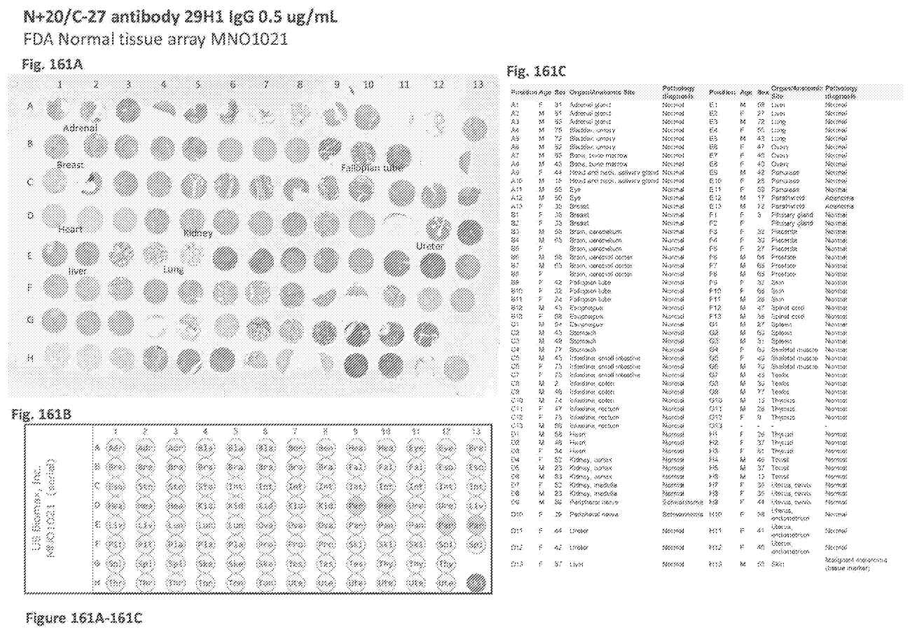

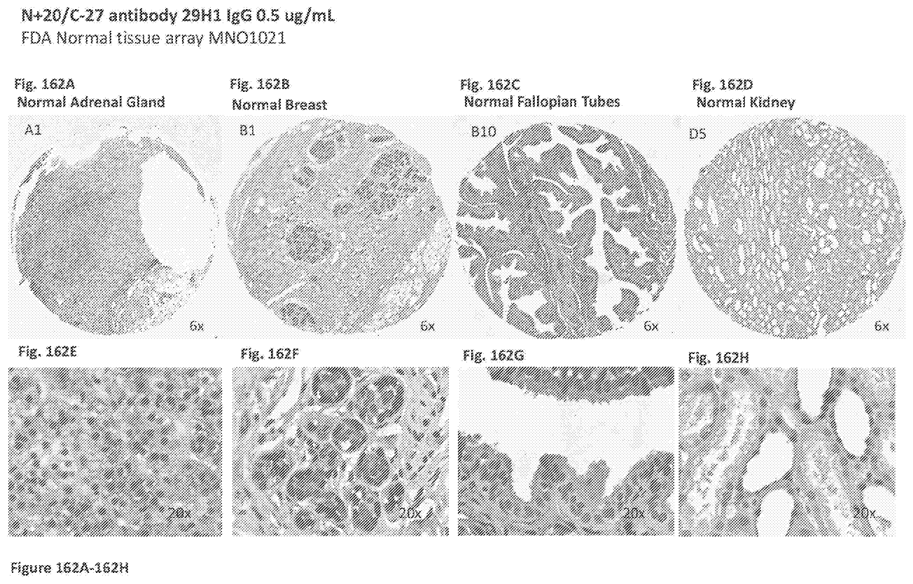

FIG. 157-158 show the binding of the N+20/C-27 antibody 1E4 to normal tissues. 1E4 is able to bind to the N-10 peptide but also is able to bind to the C-10 peptide. 1E4 binds to the QFNQYKTEA sequence which is within the PSMGFR sequence. Examination of the entire normal tissue micro array (FIG. 157A) shows that antibody 1E4 binds to many normal tissues, including brain, cerebellum, all 3 liver specimens, pancreas, parathyroid, spinal cord and skeletal muscle. Magnified images show that 1E4 stains heart (FIG. 158I) as well. 1E4 staining of a breast cancer array (FIG. 159-160) shows that there is some cancer specificity.

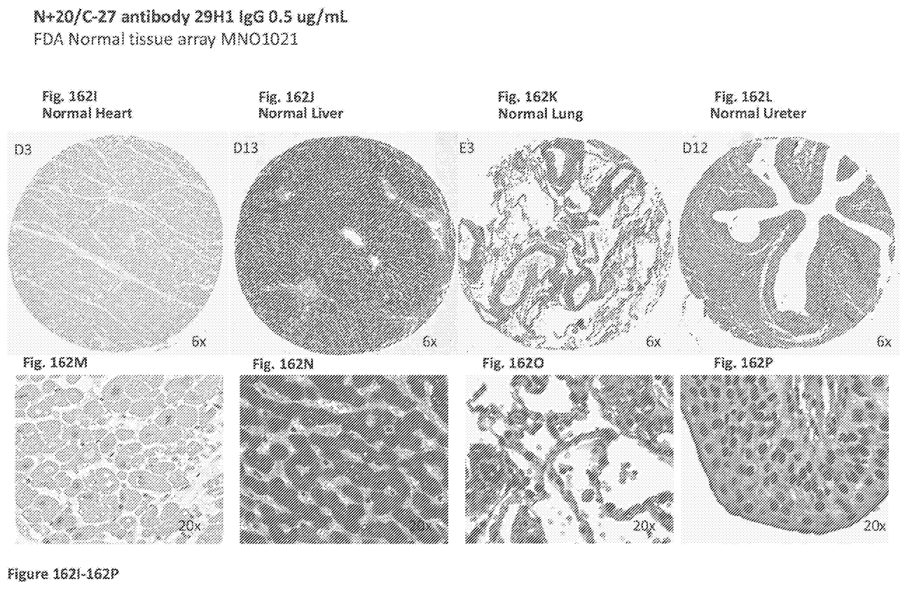

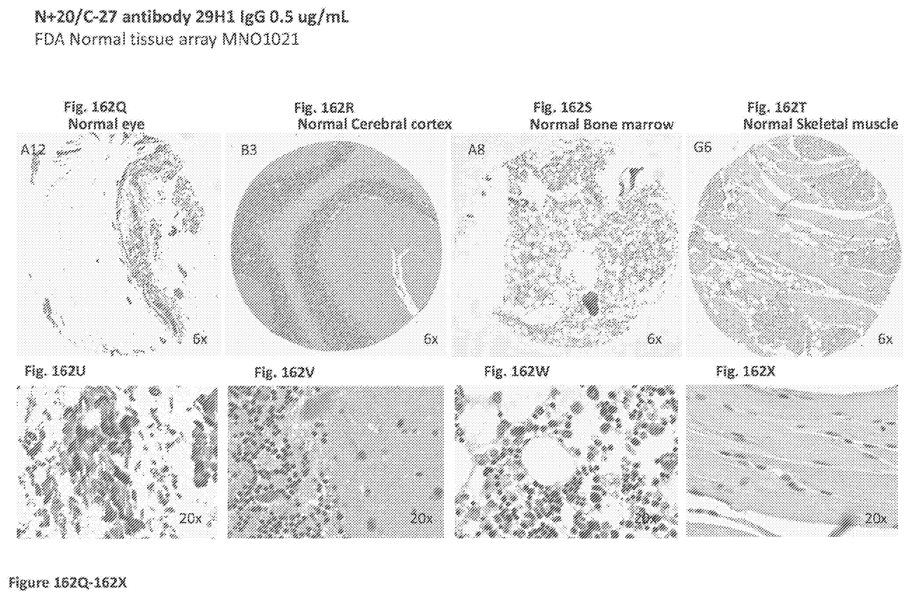

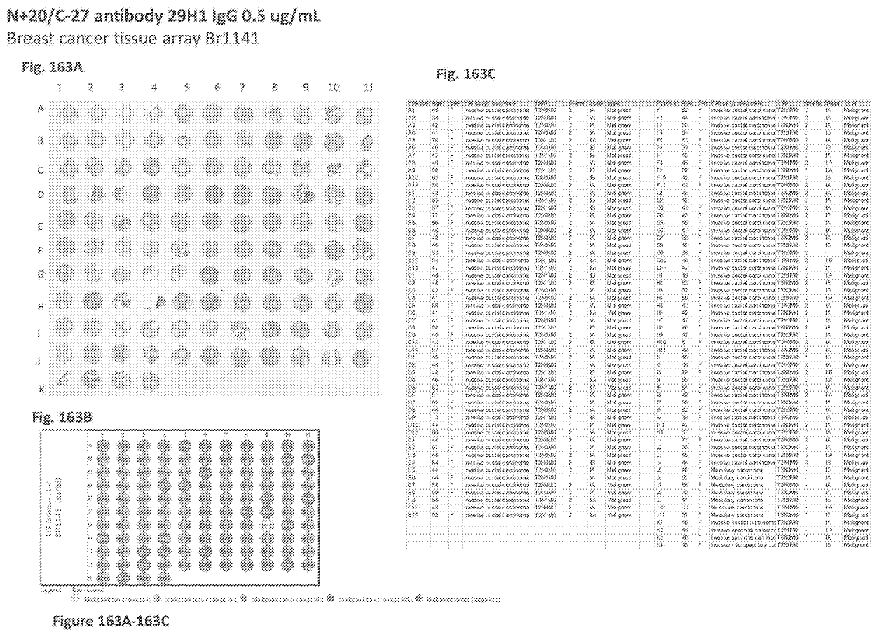

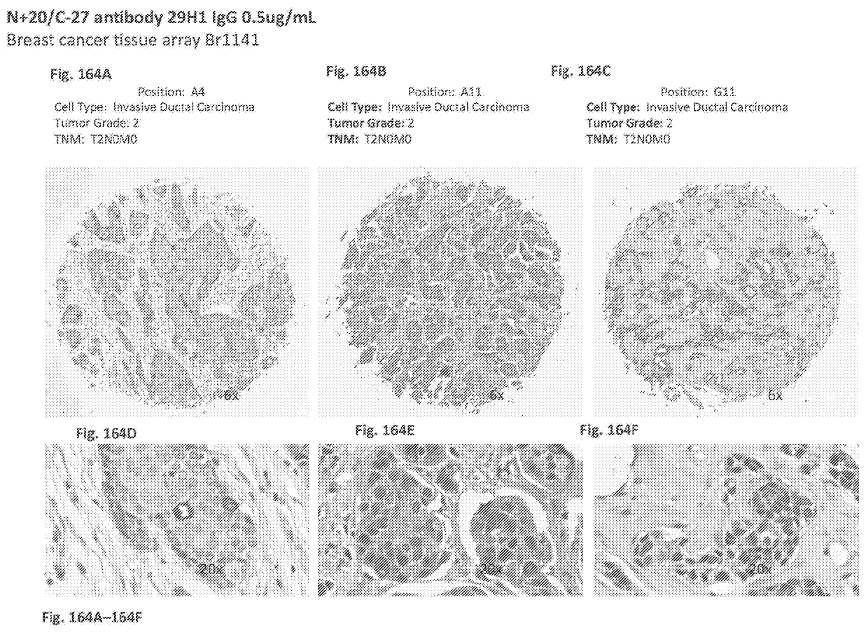

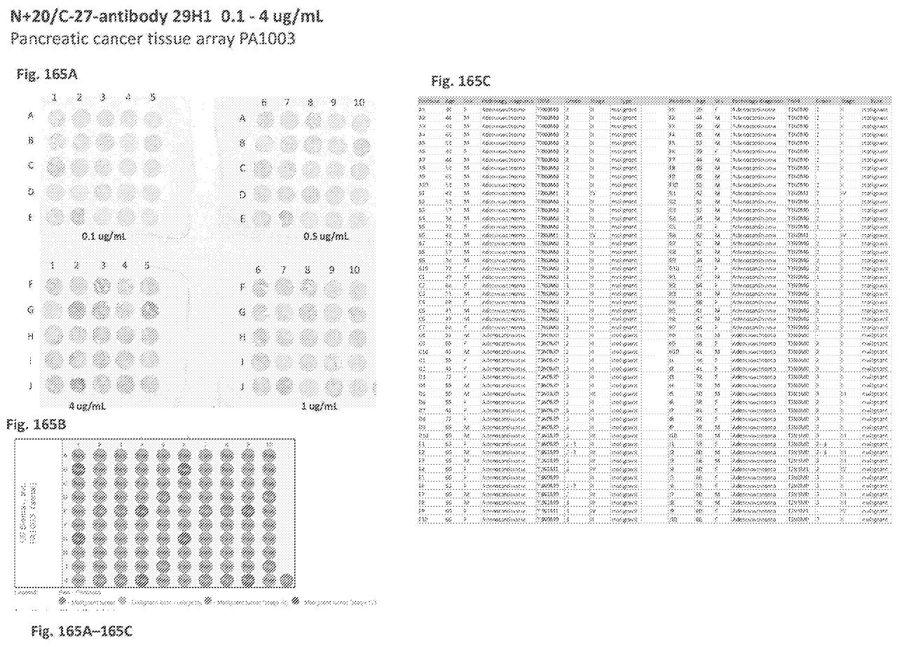

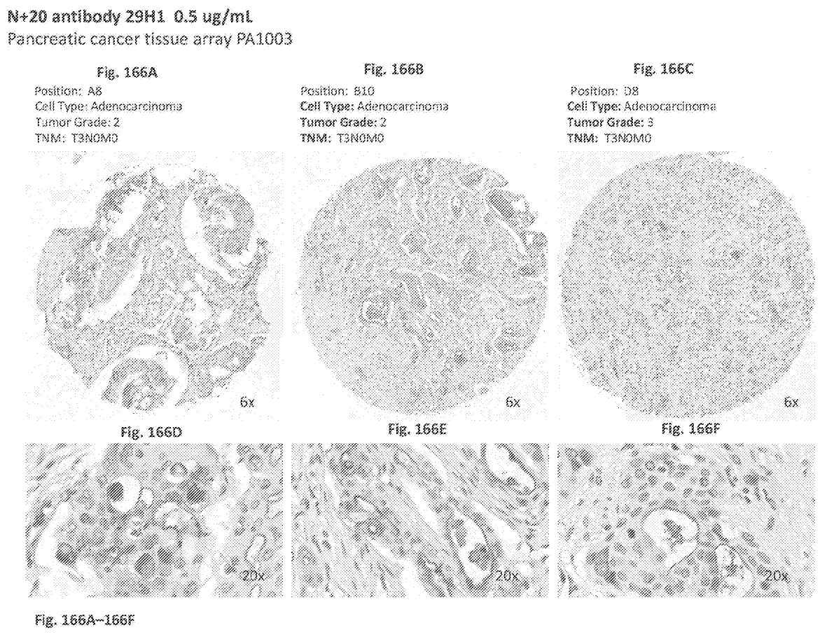

FIGS. 161-162 show the binding of the N+20/C-27 antibody 29H1 to normal tissues. 29H1 binds within the GTINVHDVET sequence, which is the most distal part of the PSMGFR sequence. Unlike antibodies MNC2, MNE6 or 20A10, 29H1 cannot bind to the N-10 peptide but does bind to the C-10 peptide. Examination of the entire normal tissue micro array (FIG. 157A) shows that even at concentration as low as 0.5 ug/mL, antibody 29H1 strongly stains a wide range of normal tissues, including brain, heart, liver and lung. 29H1 staining of a breast cancer array (FIGS. 163-164) and staining of a pancreatic cancer tissue array (FIGS. 165-166) shows that there is no cancer specificity.

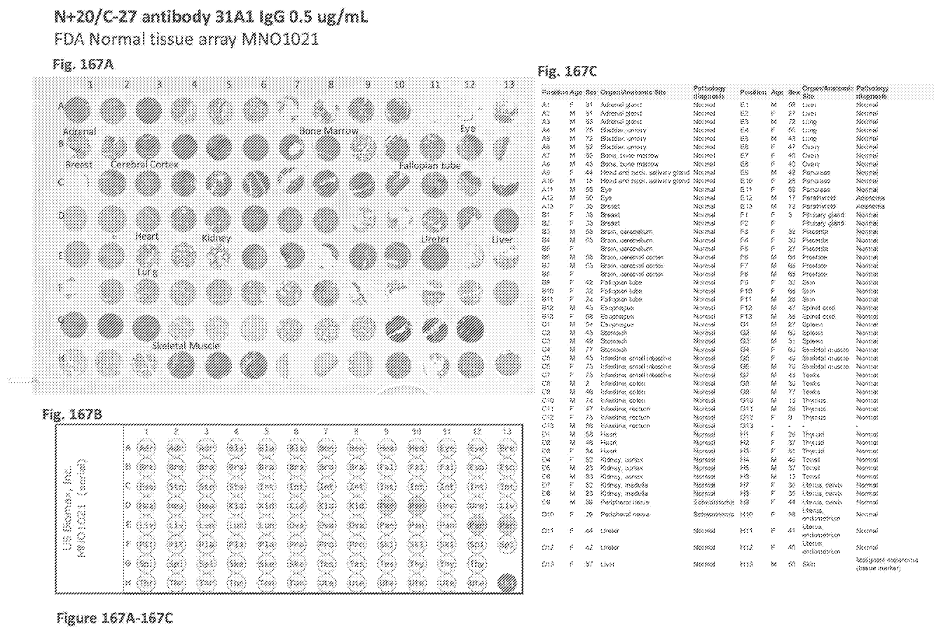

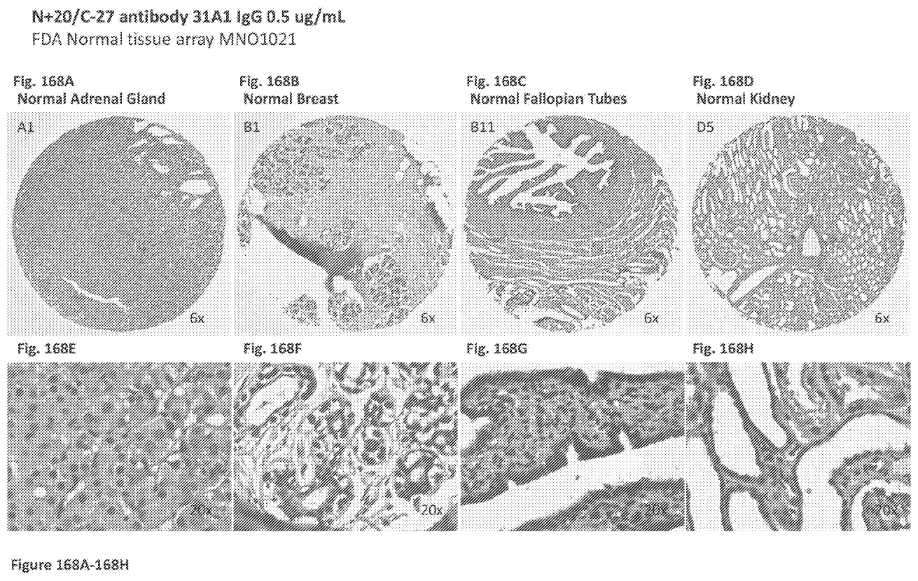

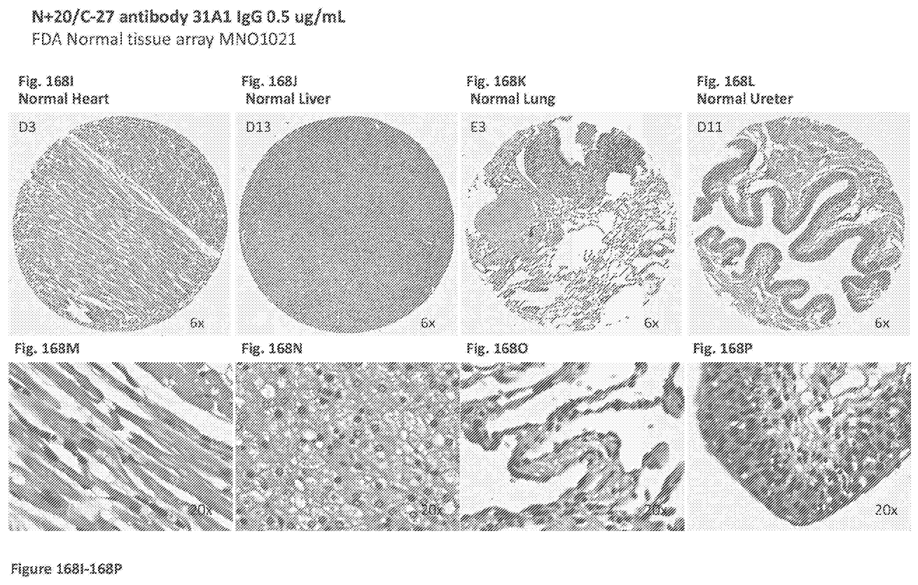

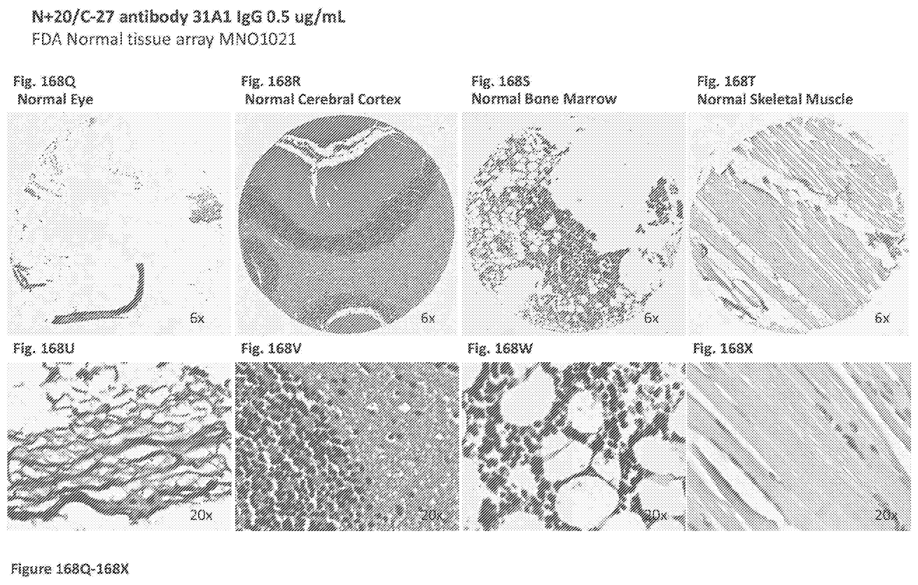

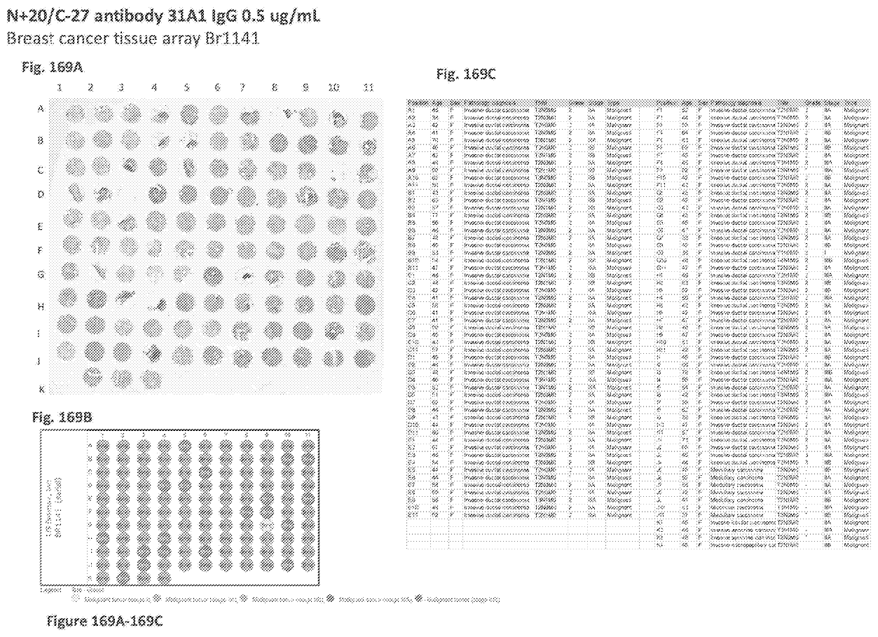

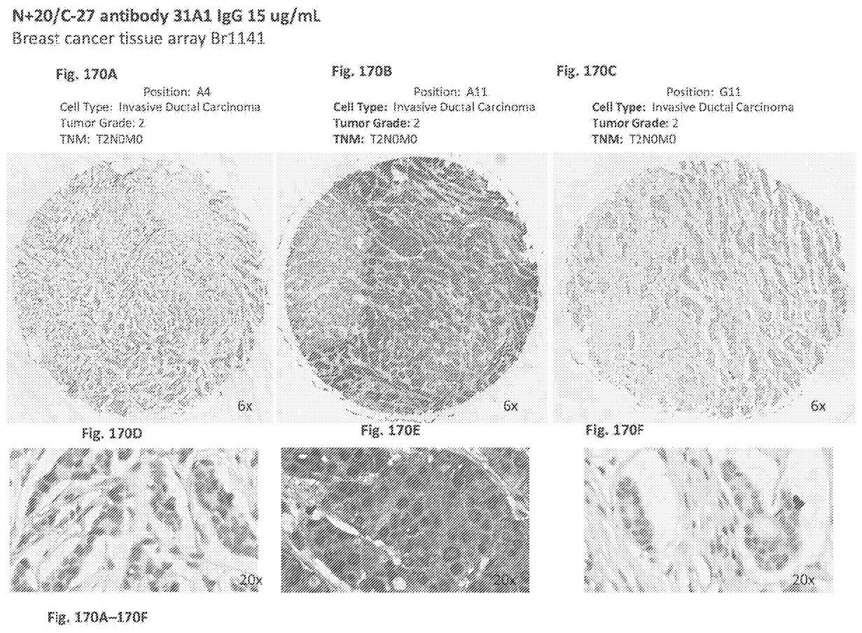

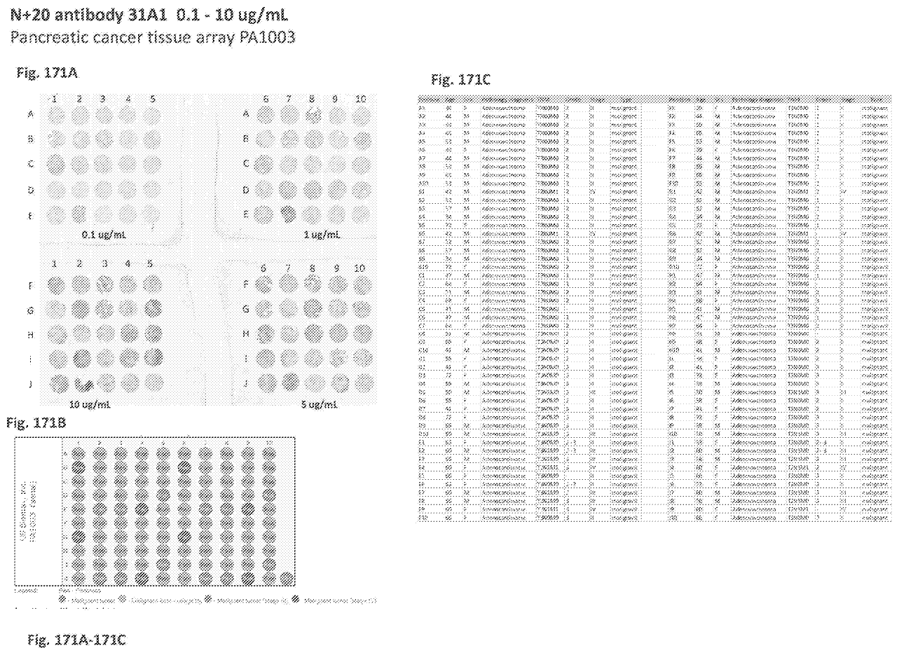

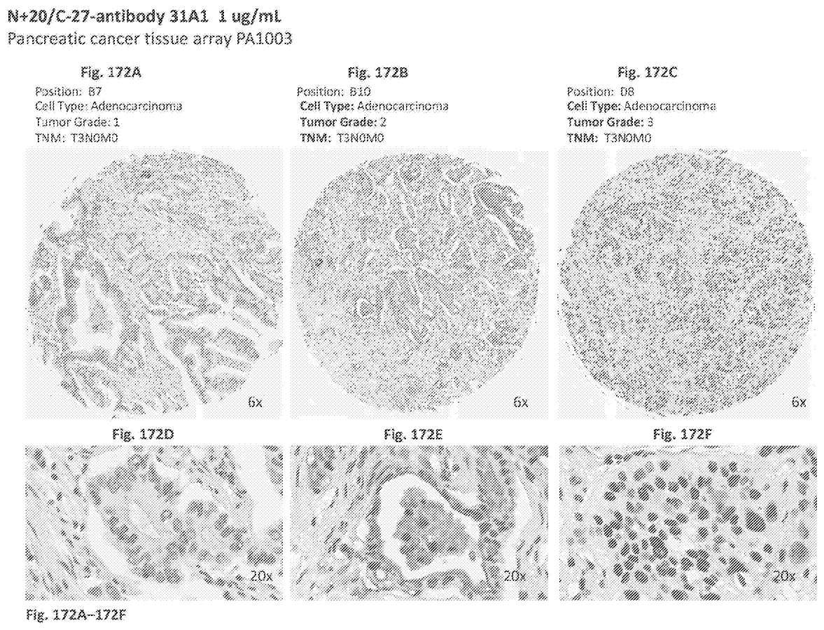

Antibody 31A1 is similar to 29H1 in that they are both N+20/C-27 antibodies that bind within the GTINVHDVET (SEQ ID NO: 1746) sequence, which is the most distal part of the PSMGFR sequence. Unlike antibodies MNC2, MNE6 or 20A10, neither 31A1 nor 29H1 can bind to the N-10 peptide but do bind to the C-10 peptide. Examination of the entire normal tissue micro array and the magnified images (FIGS. 167-168) shows that even at concentration as low as 0.5 ug/mL, antibody 31A1 strongly stains a wide range of normal tissues, including brain, heart, lung, spleen, bone marrow, and skeletal muscle. 31A1 was used to stain a breast cancer array, (FIGS. 169-170). 31A1 was used over a range of concentrations to stain a pancreatic cancer tissue array (FIGS. 171-172). These figure shows that 31A1 has insufficient cancer specificity.

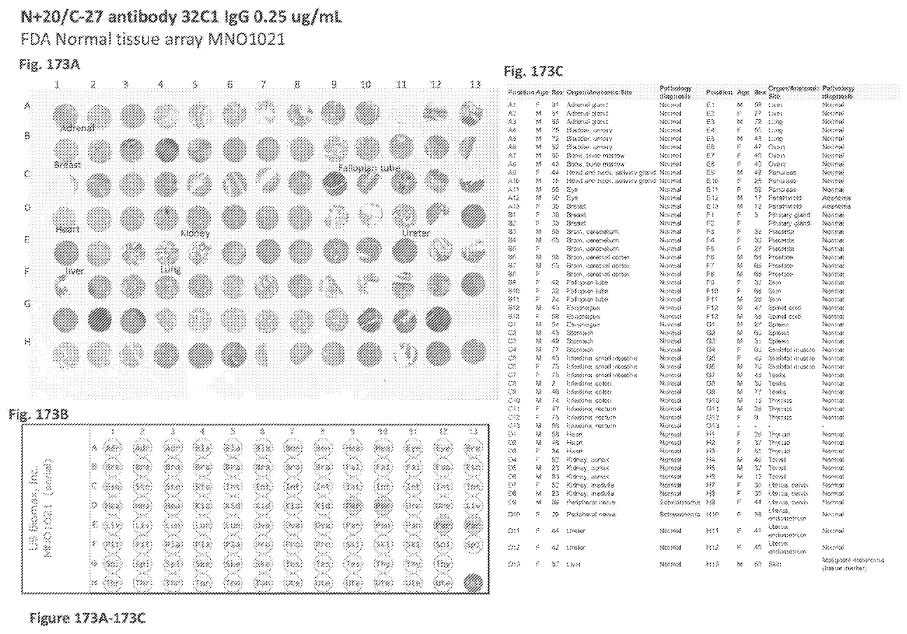

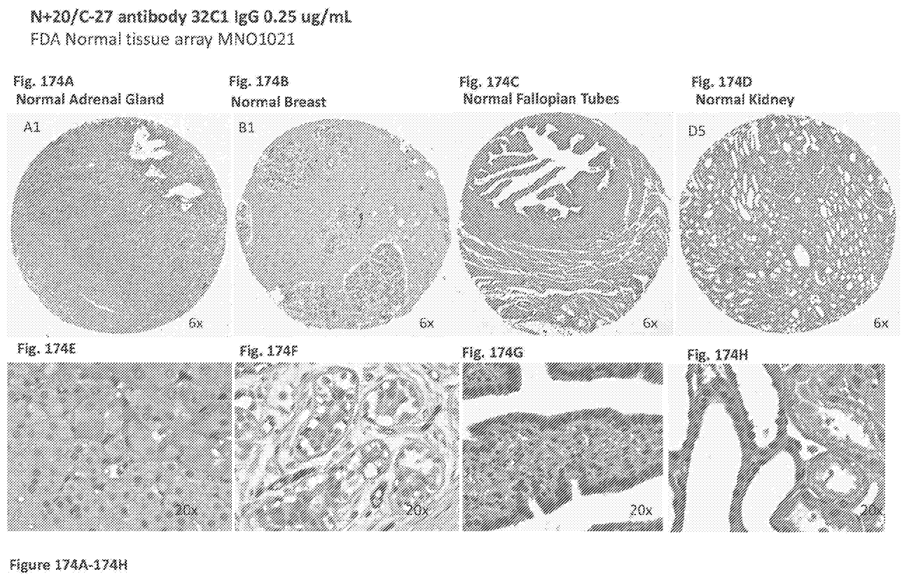

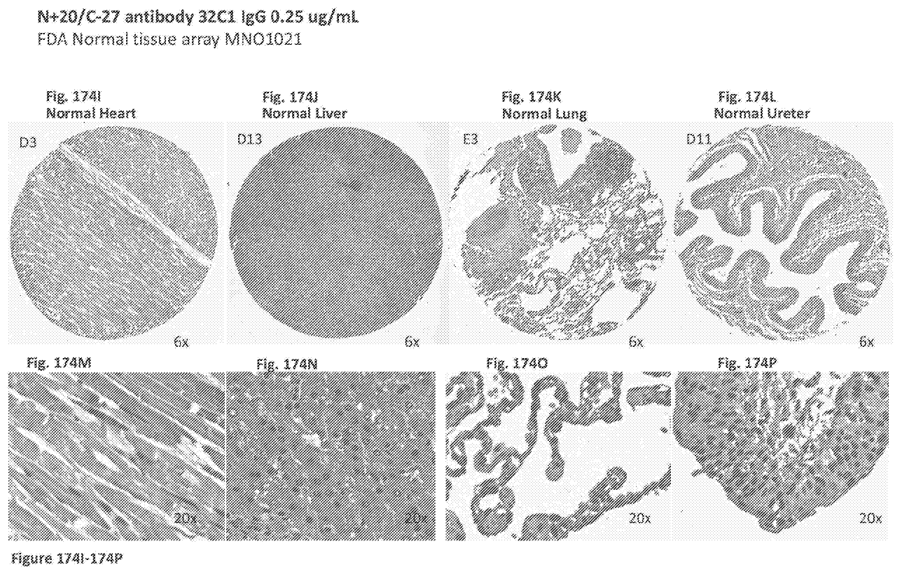

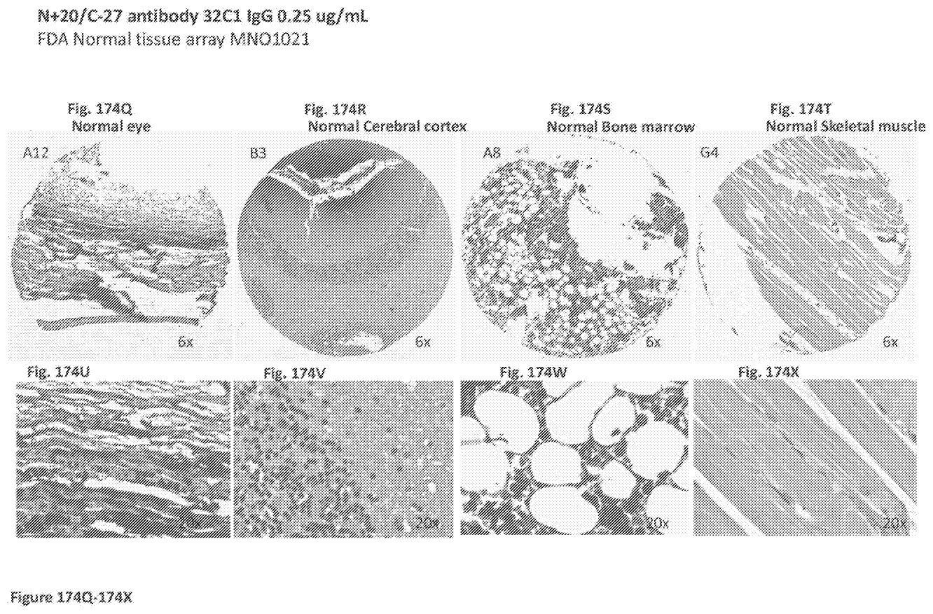

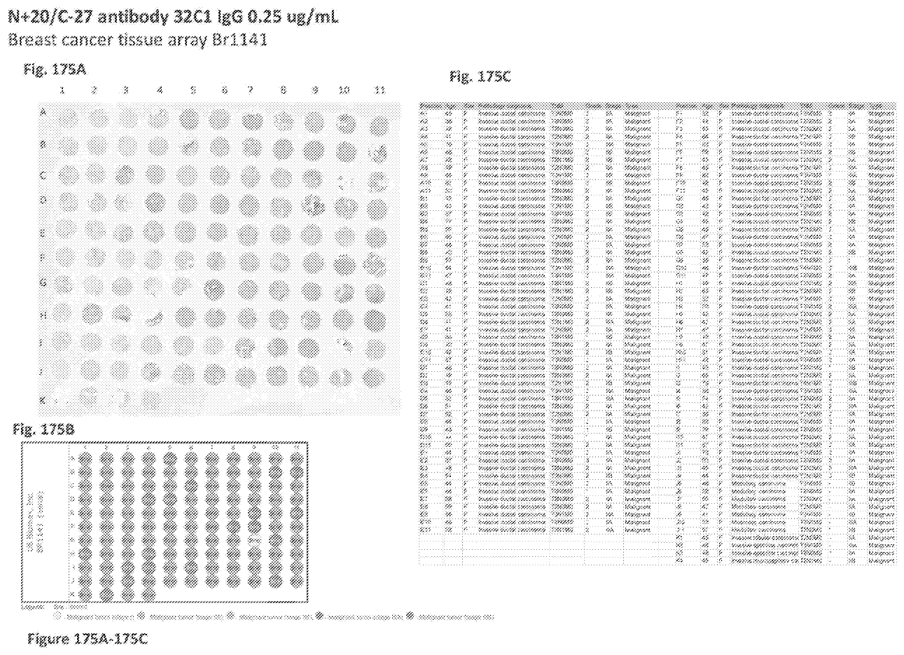

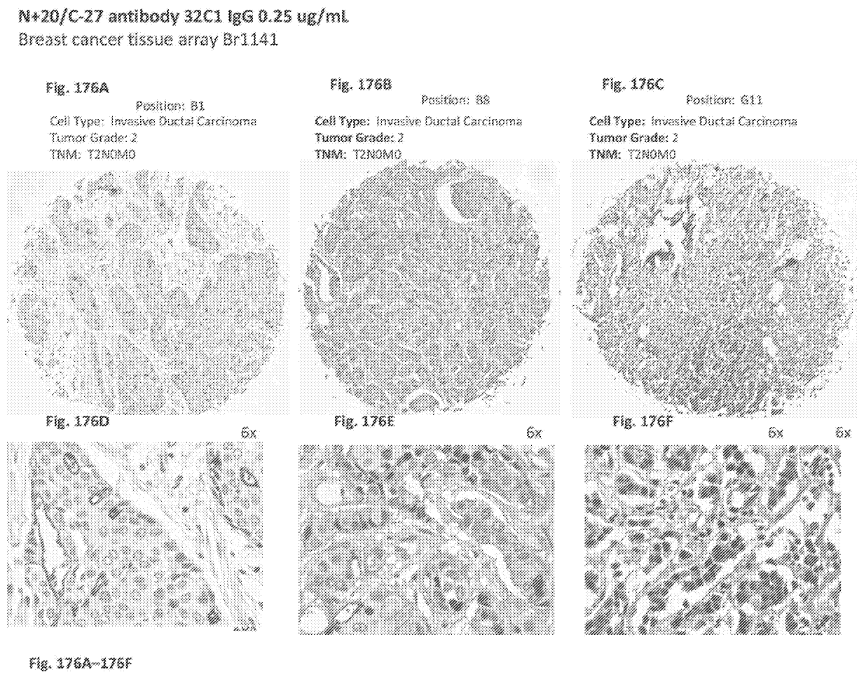

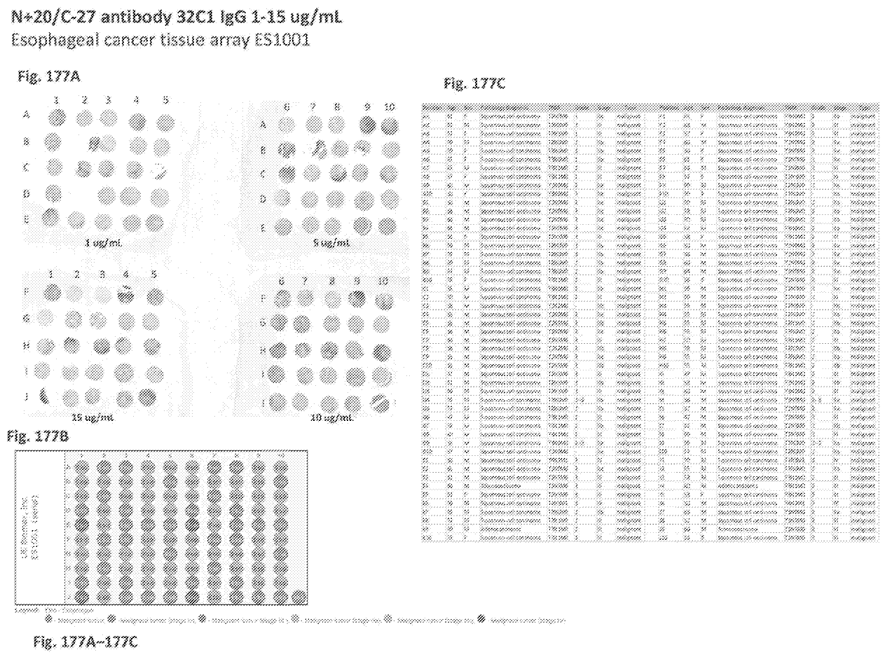

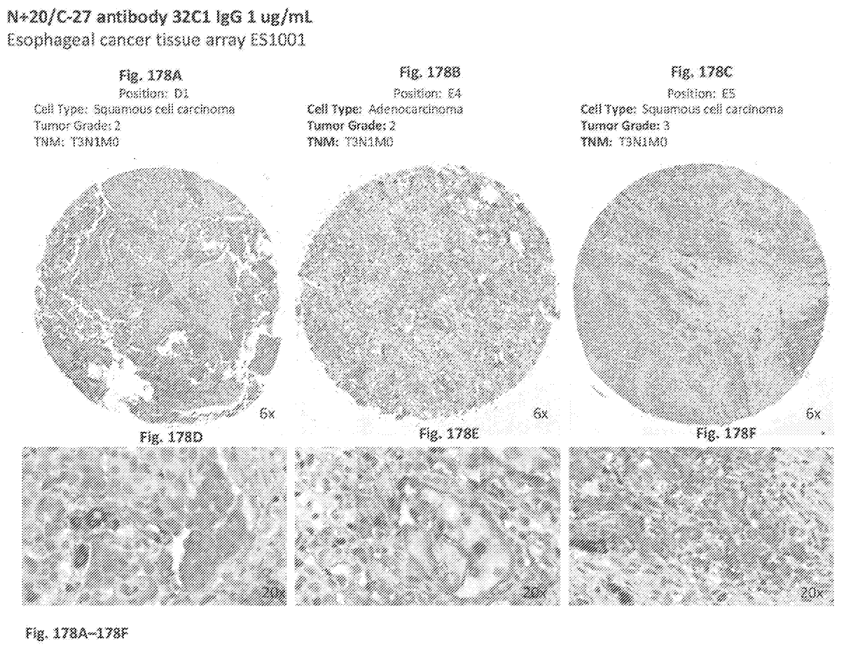

Antibody 32C1 is similar to 29H1 and 31A1 in that they are all N+20/C-27 antibodies that bind within the GTINVHDVET sequence, which is the most distal part of the PSMGFR sequence. Unlike antibodies MNC2, MNE6 or 20A10, none of 32C1, 31A1 or 29H1 can bind to the N-10 peptide but all do bind to the C-10 peptide. Examination of the entire normal tissue micro array and the magnified images (FIG. 173-174) shows that even at concentration as low as 0.25 ug/mL, antibody 32C1 strongly stains a wide range of normal tissues, including brain, heart, lung, liver, spleen and bone marrow. 32C1 was also used to probe a breast cancer array (FIG. 175-176). 32C1 was used over a range of concentrations to stain an esophageal cancer tissue array (FIG. 177-178). Taken together, these figures show that 32C1 has insufficient cancer specificity.

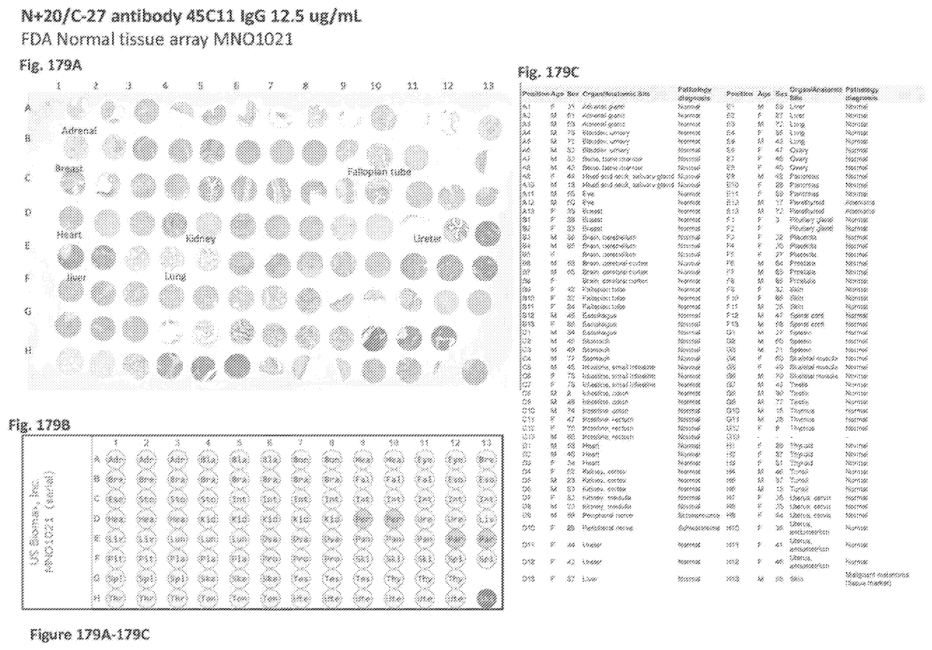

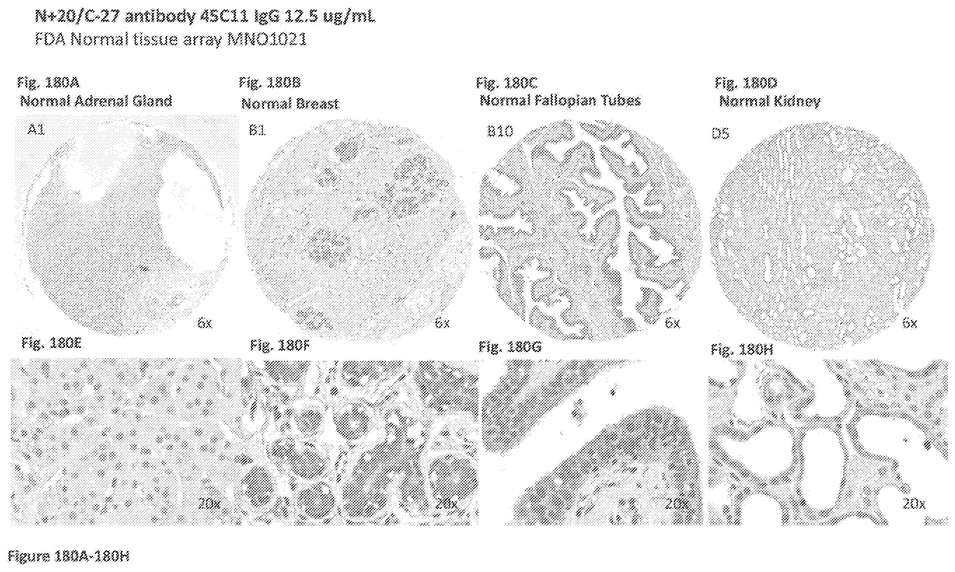

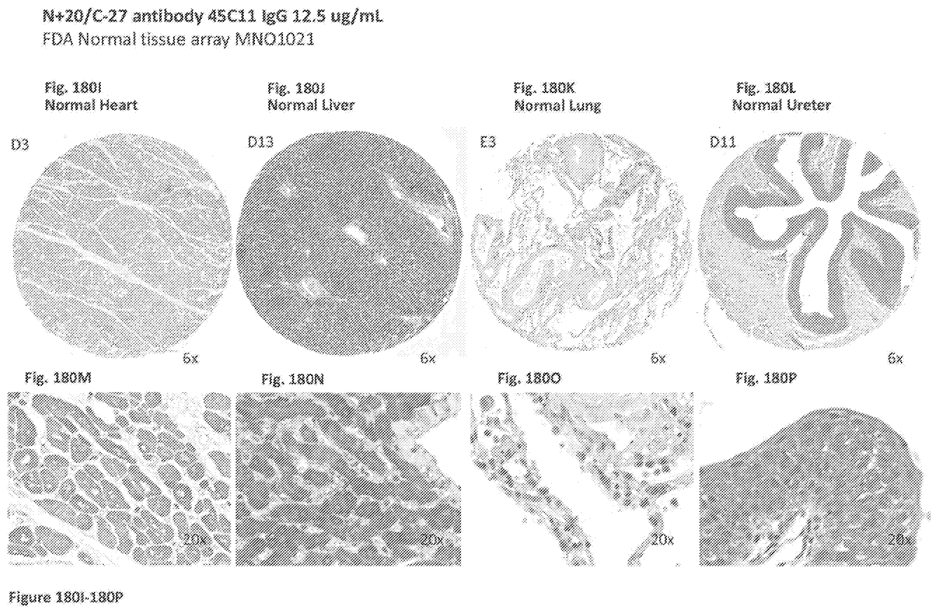

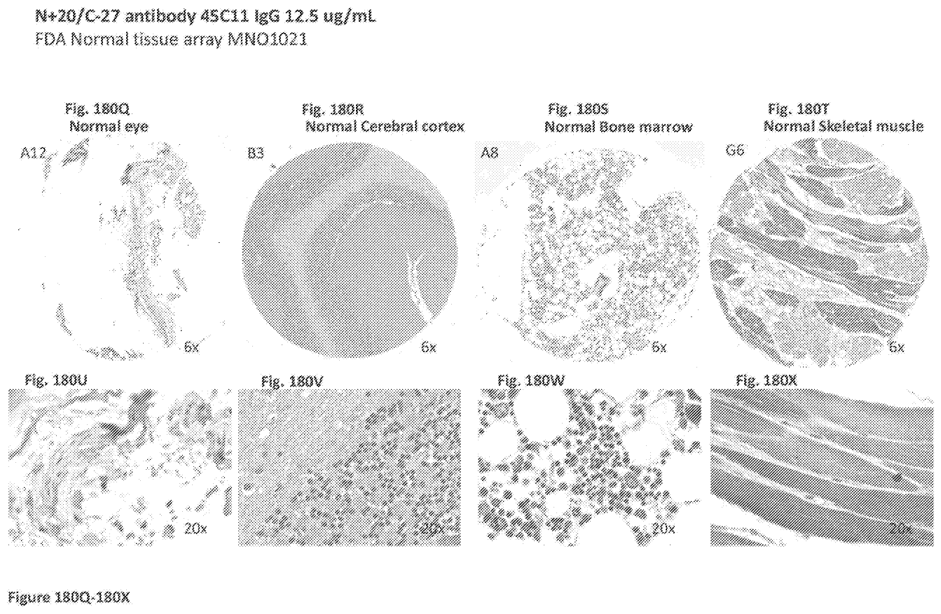

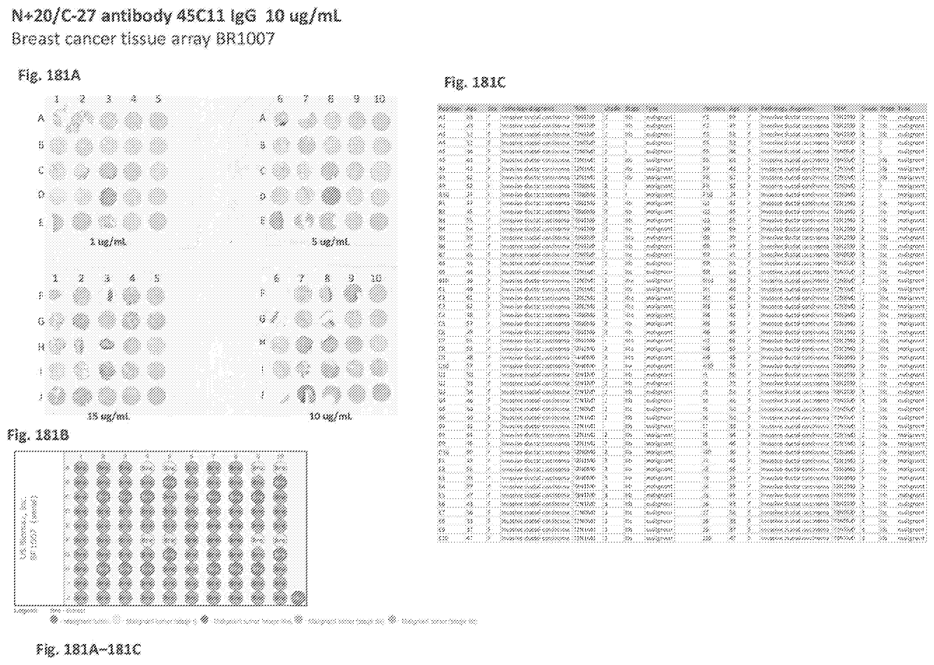

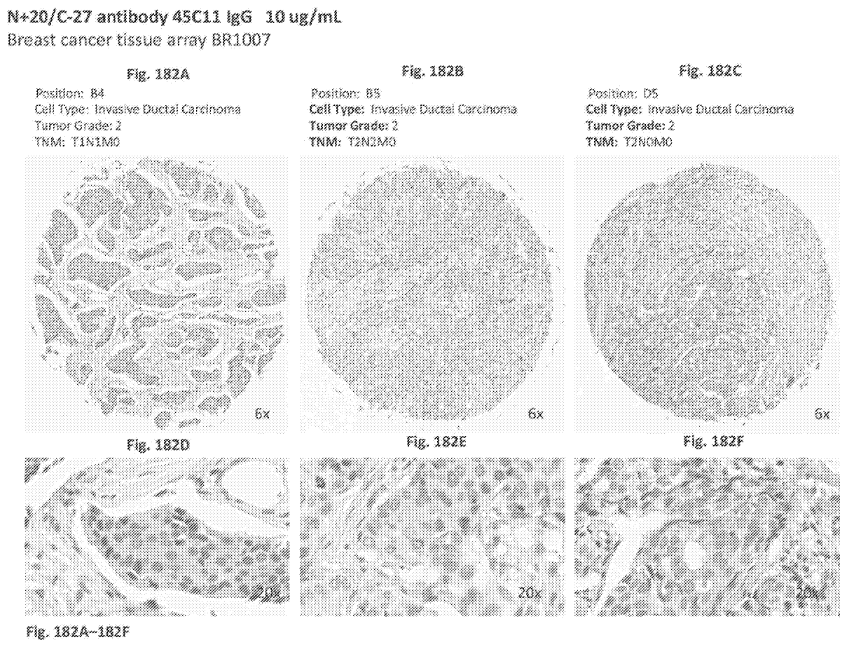

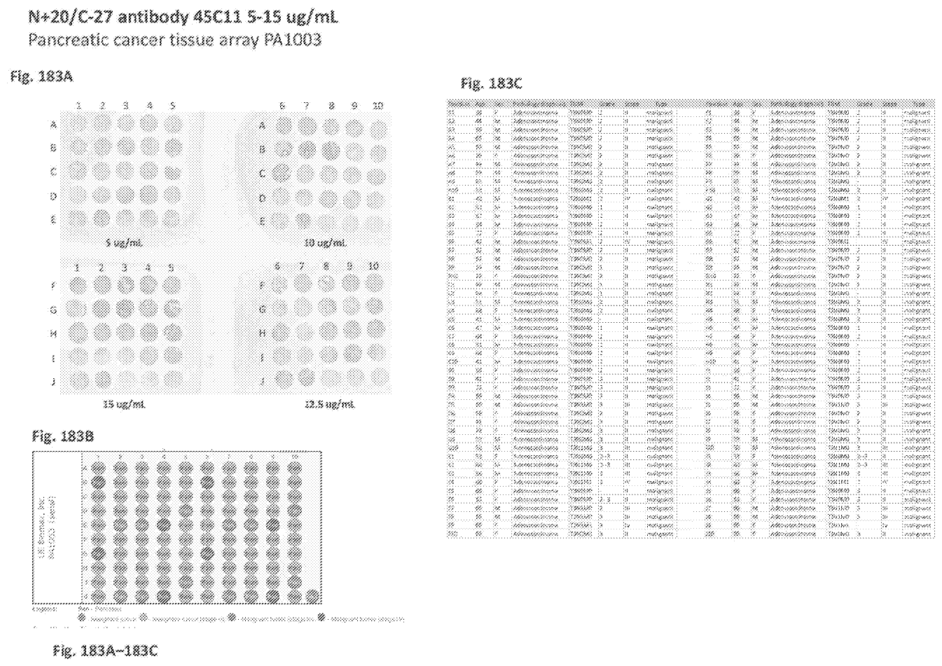

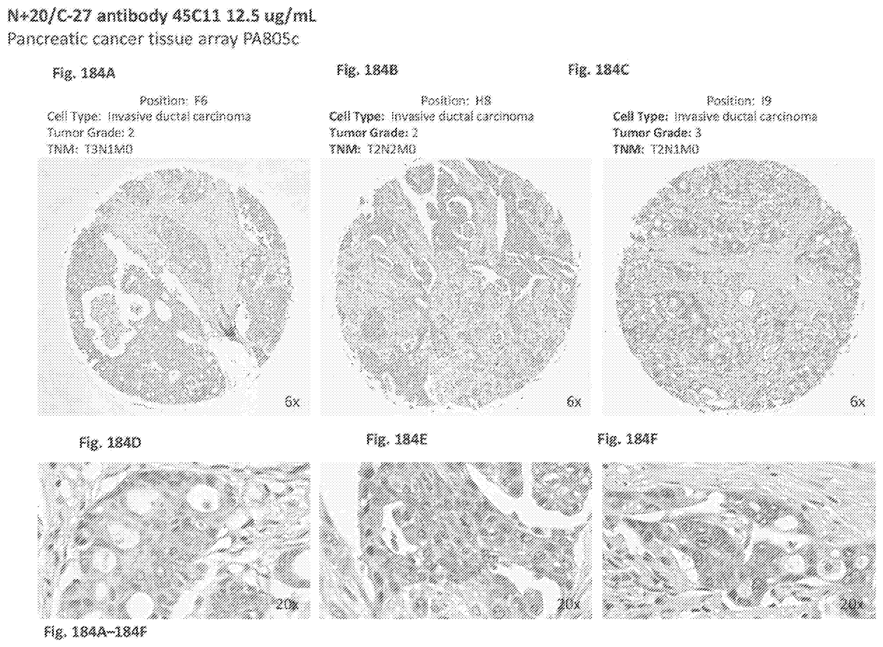

Antibody 45C11 is an N+20/C-27 antibody that binds to epitope SNIKFRPGSVV (SEQ ID NO: 1744) that is 20 amino acids outside of the PSMGFR sequence at the N-terminal end. 45C11 does not bind to the N-10 peptide. Normal tissue array FDA MNO1021 was stained with 45C11 at 12.5 ug/mL (FIG. 179-180). As can be seen in the figures, 45C11 shows strong binding to many normal tissues, including brain, heart, lung, liver, spleen, skeletal muscle and bone marrow. 45C11 was used over a range of concentrations to stain a breast cancer tissue array (FIG. 181-182). 45C11 was also used to stain a pancreatic cancer tissue array (FIG. 183-184). Taken together, these figures show that 45C11 has no cancer specificity.

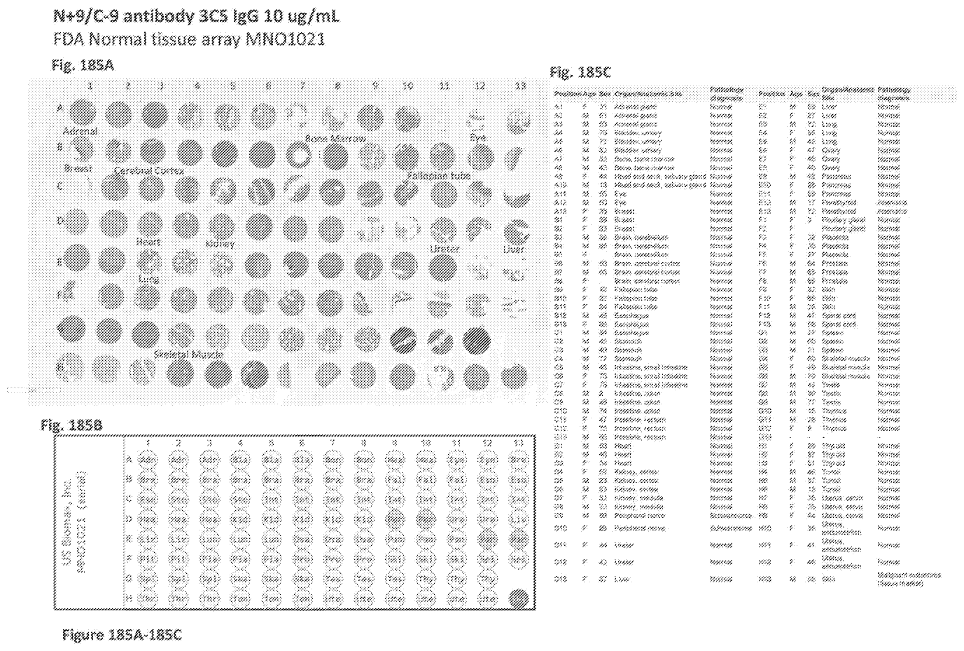

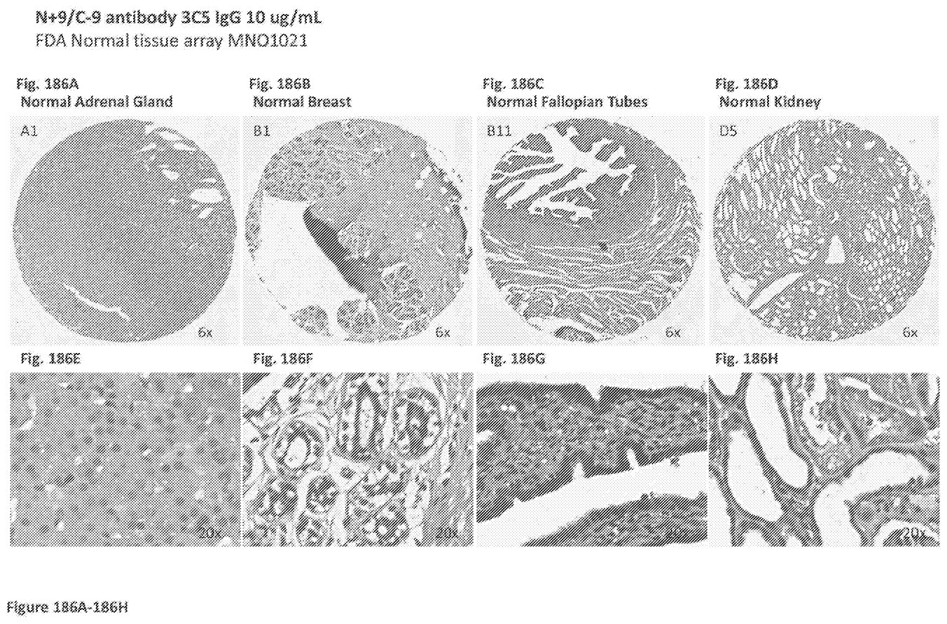

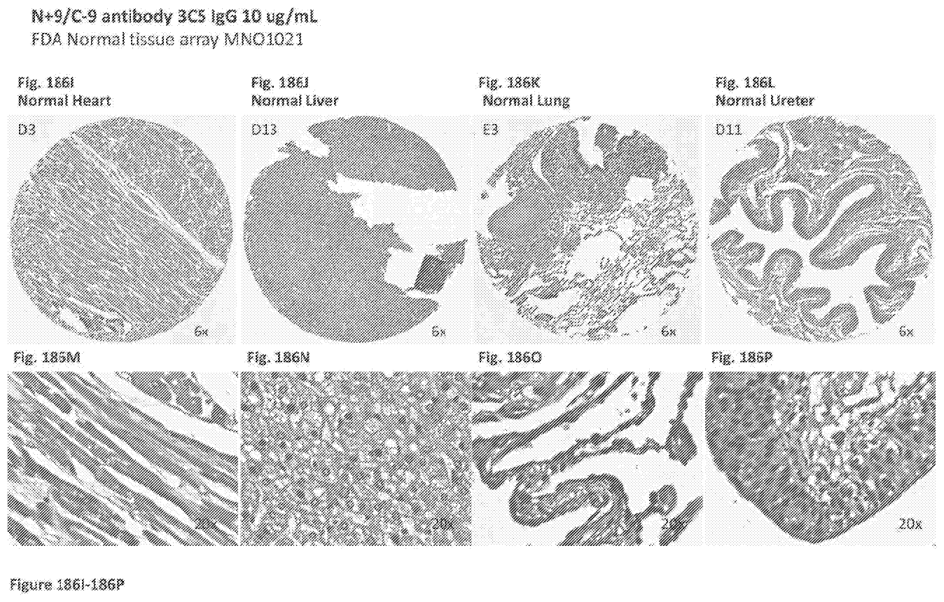

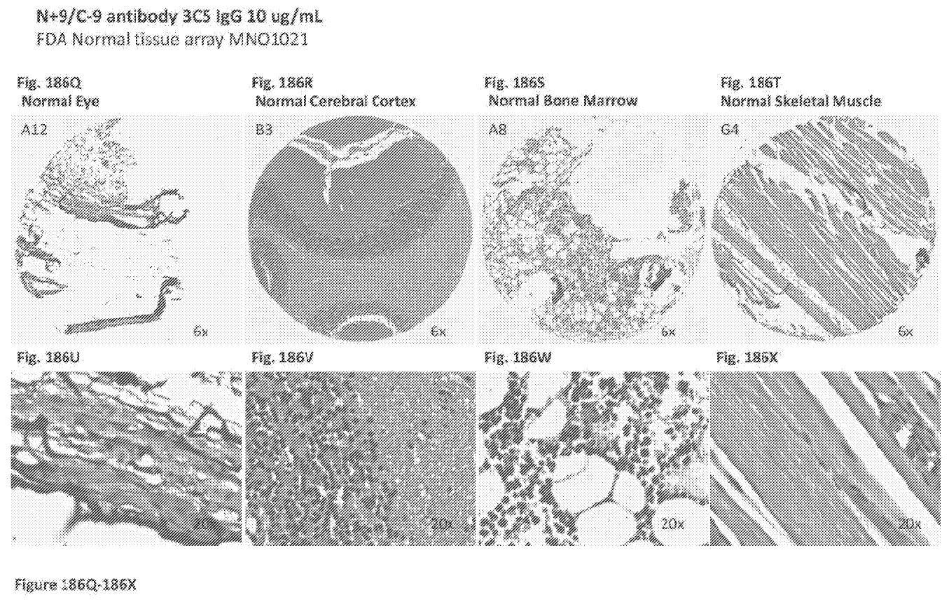

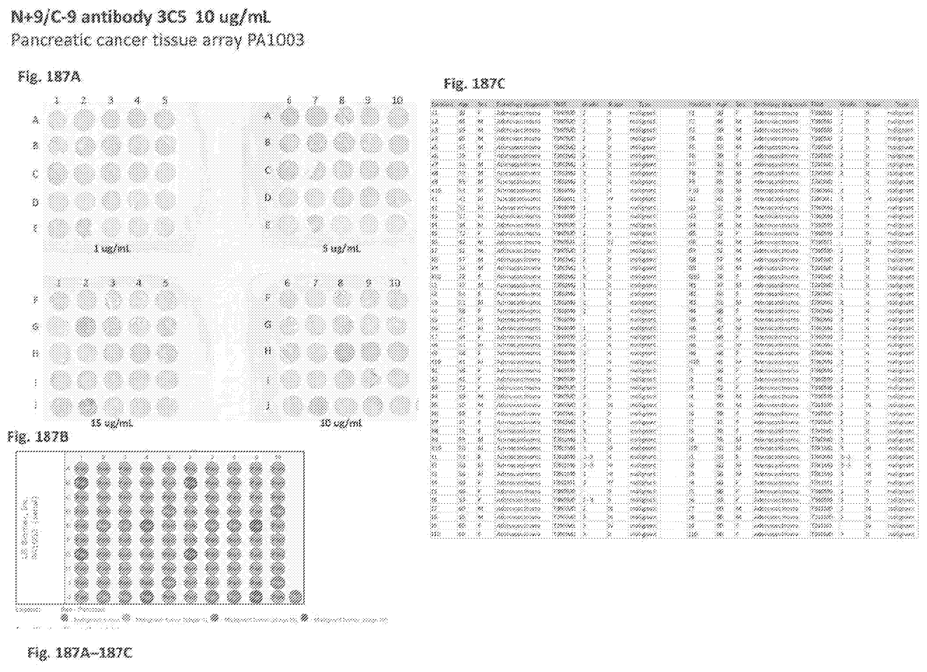

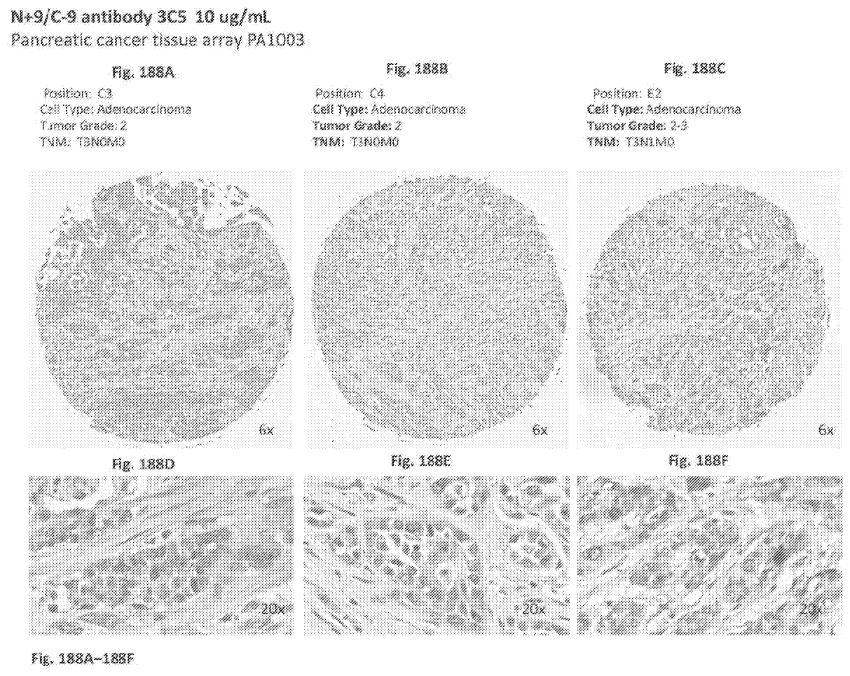

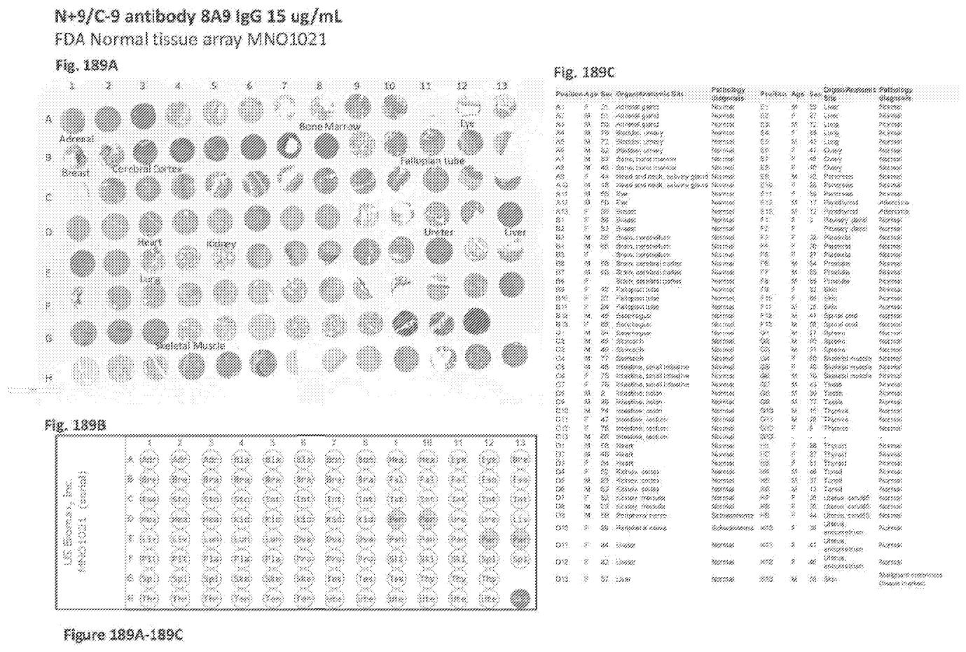

Antibody 3C5 is an N+9/C-9 antibody that binds to epitope GTINVHDVET. Like the other antibodies that bind to this epitope such as 32C1, 29H1 and 31A1, they bind to the most distal, that is to say the most N-terminal, part of the PSMGFR sequence. Unlike antibodies MNC2, MNE6 or 20A10, none of 3C5, 32C1, 31A1 or 29H1 can bind to the N-10 peptide but all do bind to the C-10 peptide. Examination of the entire normal tissue micro array, where 3C5 was used at 10 ug/mL, and the magnified images (FIG. 185-186) shows that antibody 3C5 strongly stains some normal tissues, including brain, heart, adrenal gland and bone marrow. 3C5 was also used to probe a pancreatic cancer array at 10 ug/mL, (FIG. 187-188). Taken together, these figures show that 3C5 has no cancer specificity.

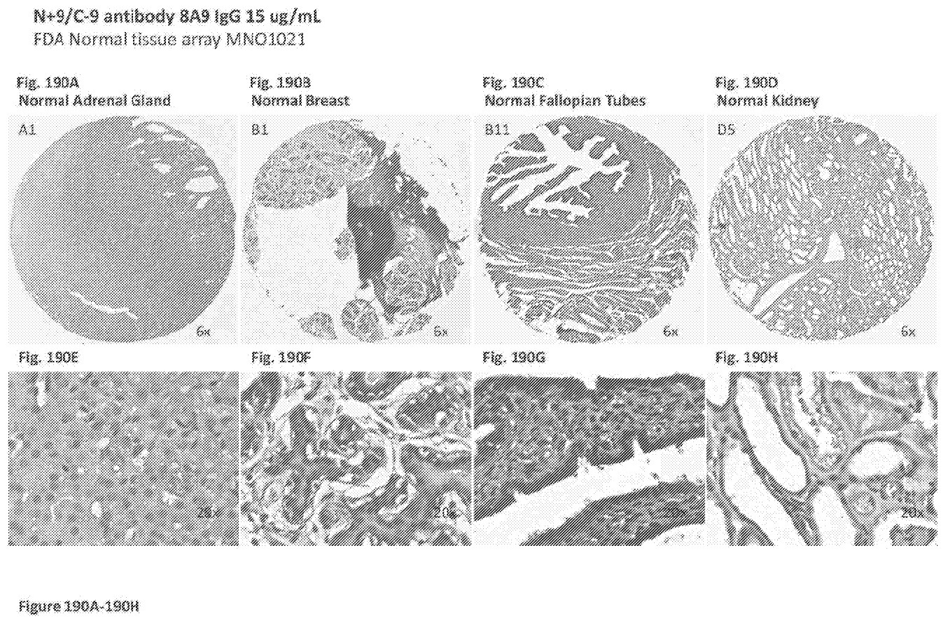

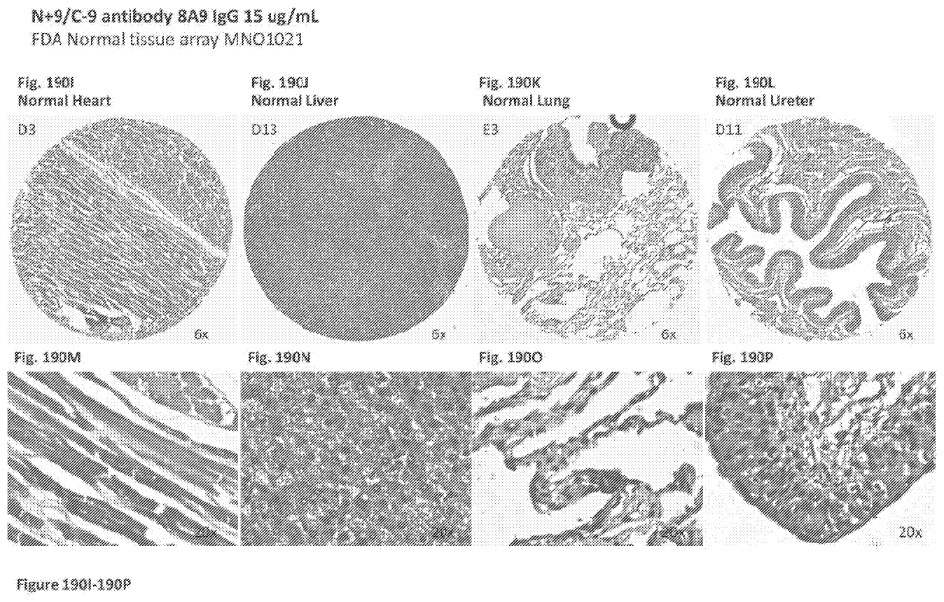

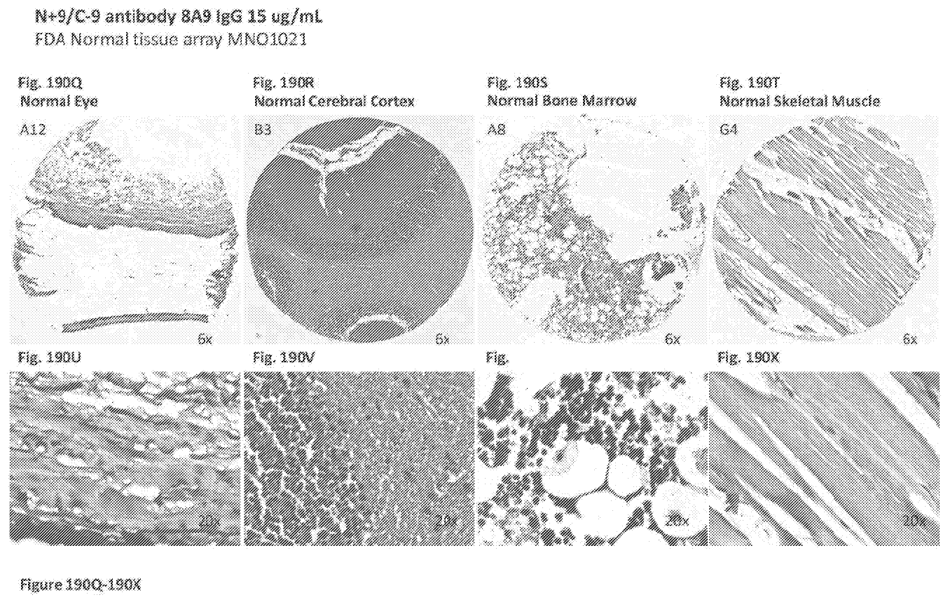

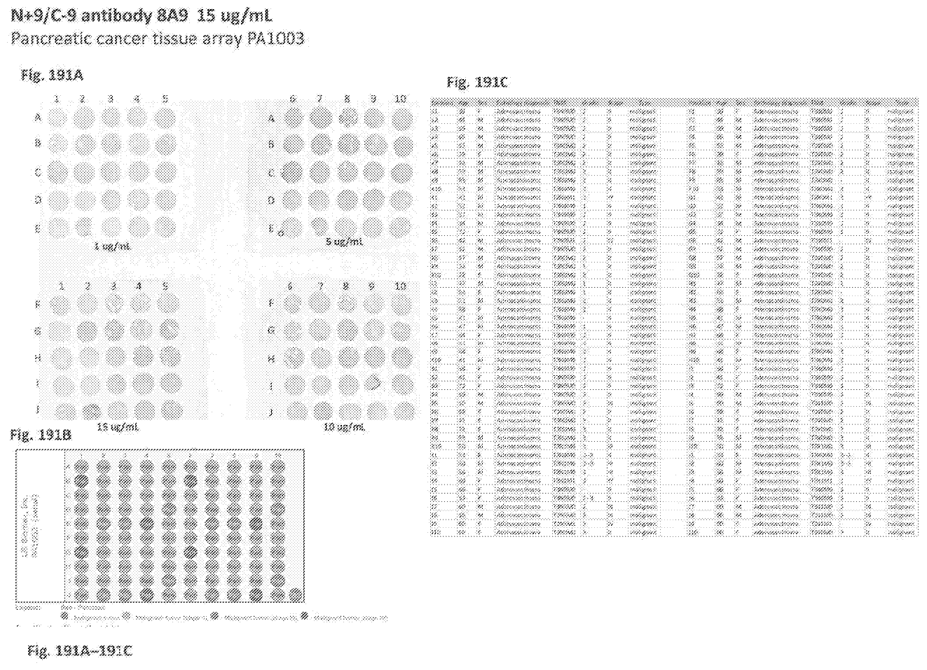

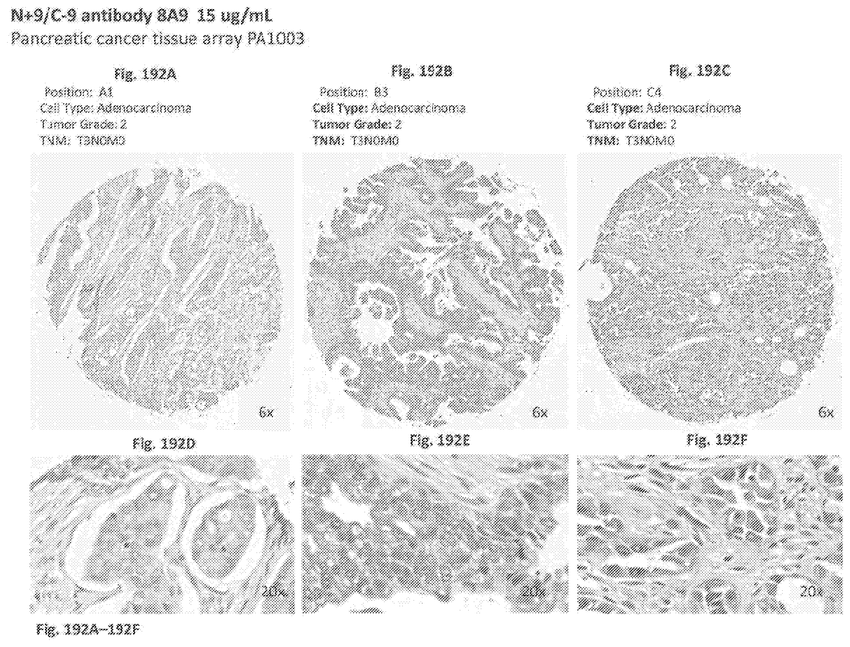

Antibody 8A9 is an N+9/C-9 antibody that binds to epitope VQLTLAFRE which is outside of the PSMGFR sequence. Antibody 8A9 cannot bind to the N-10 peptide. Normal tissue array FDA MNO1021 was stained with 8A9 (FIG. 189-190). As can be seen in the figures, like antibody 45C11, which also binds an epitope that is N-terminal beyond the PSMGFR sequence, antibody 8A9 shows strong binding to many normal tissues, including adrenal, brain, heart, lung, liver, spleen, skeletal muscle and bone marrow. A pancreatic cancer array stained with antibody 8A9 showed weak binding to a subset of pancreatic cancer tissues (FIG. 191-192). Taken together, these figures show that 8A9 has no cancer specificity.

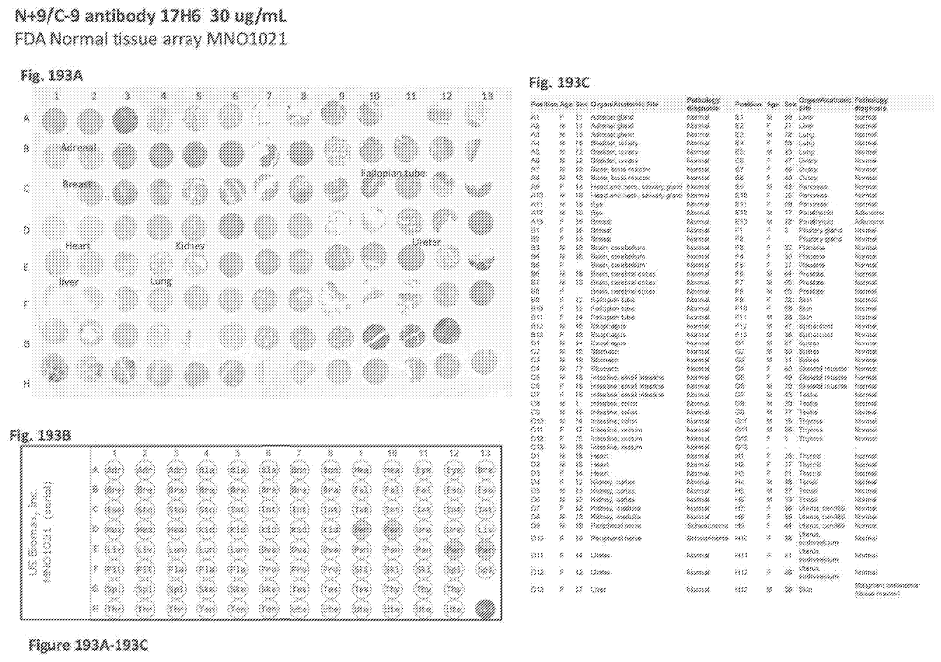

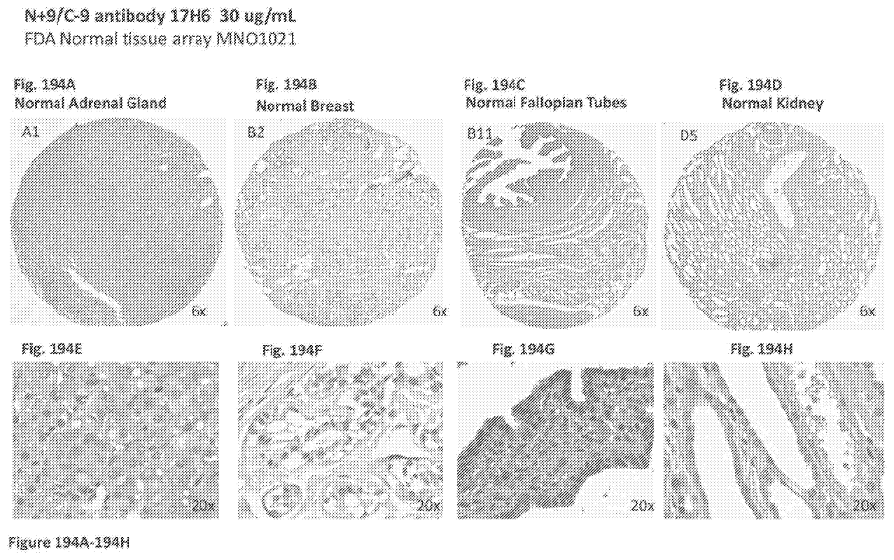

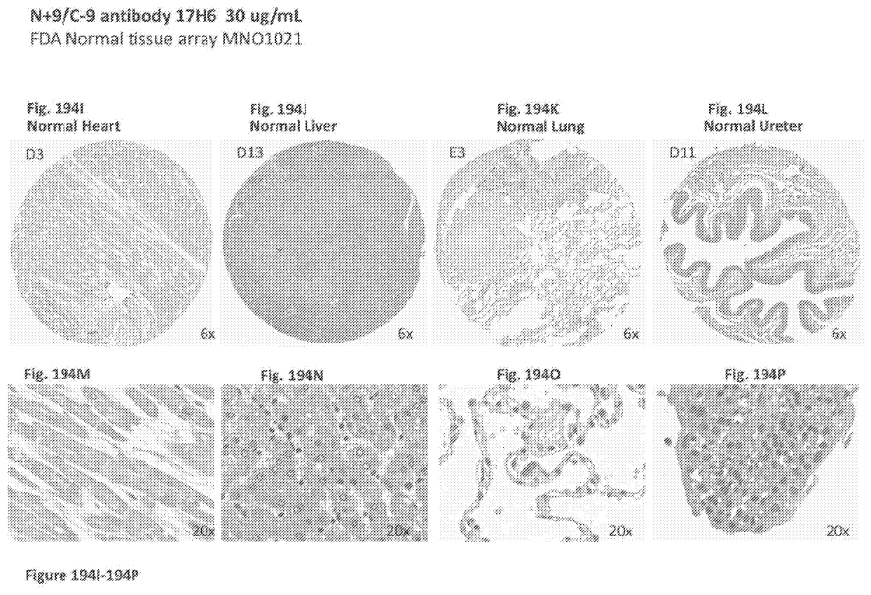

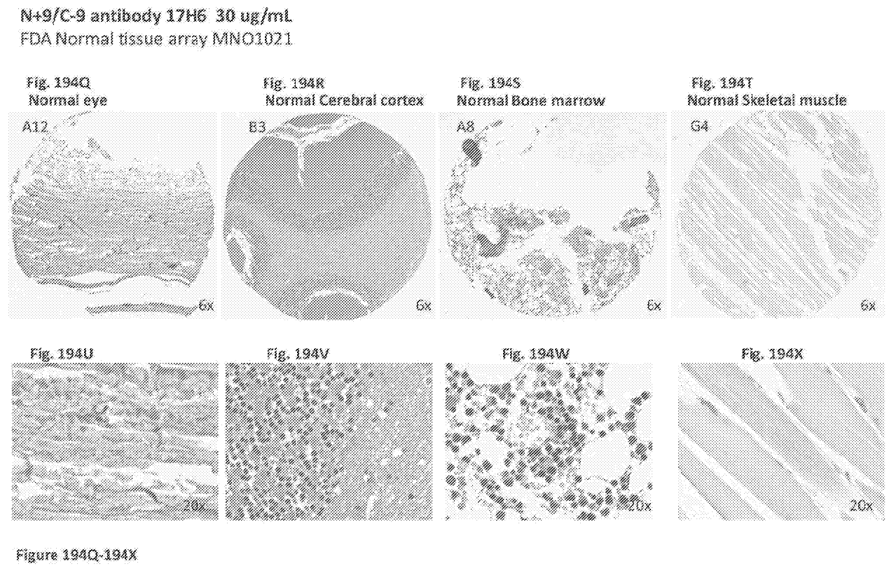

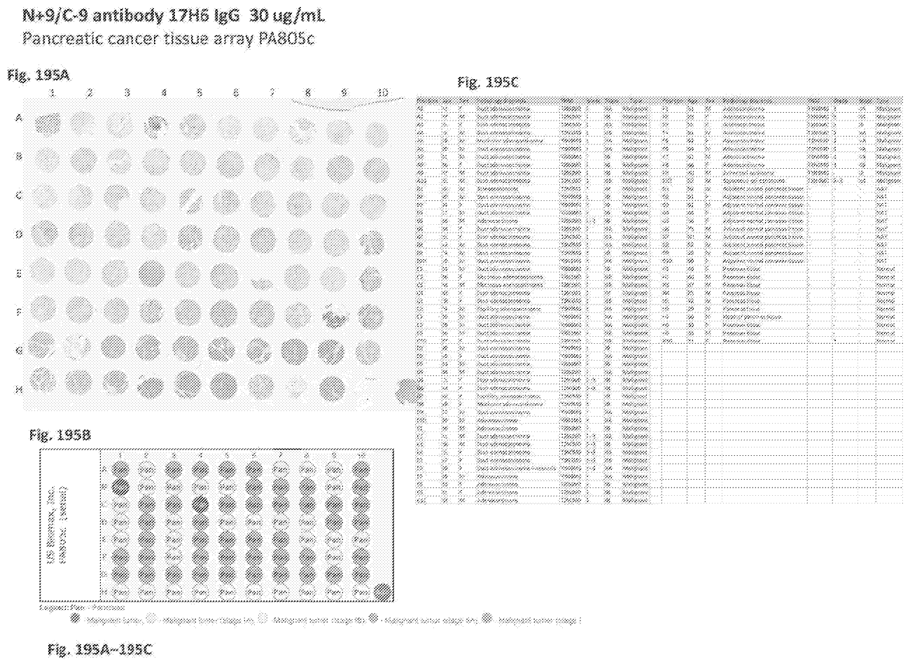

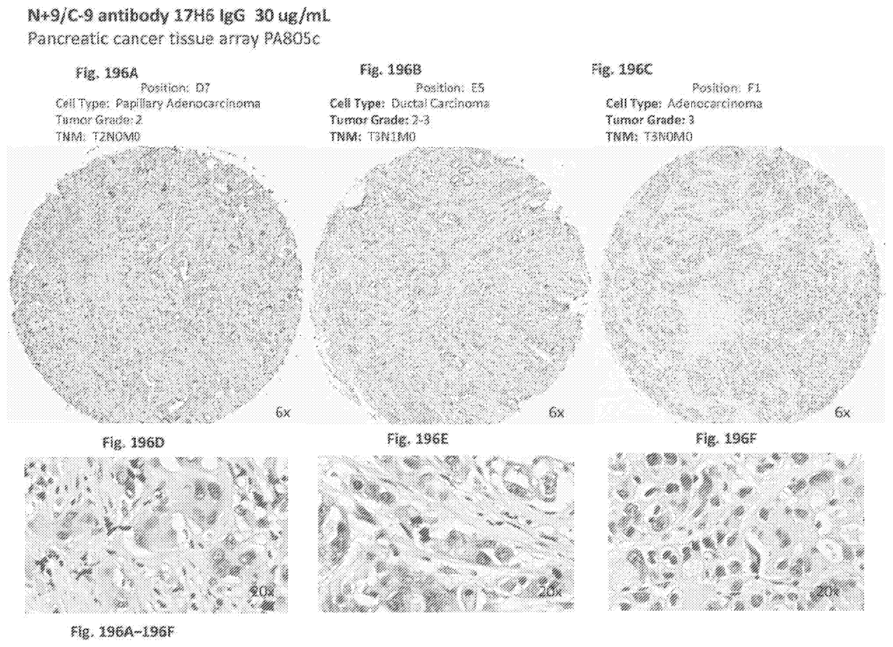

Antibody 17H6 is an N+9/C-9 antibody that binds to epitope VQLTLAFRE, which is outside of the PSMGFR sequence. 17H6 was used to stain normal tissue array MNO1021. Examination of the entire normal tissue micro array and the magnified images (FIG. 193-194) shows that antibody 17H6 stains some normal tissues, including brain, heart, adrenal gland, bone marrow and skeletal muscle. 17H6 was used to probe a pancreatic cancer array and showed weak binding to most pancreatic cancer tissues (FIG. 195-196). However, the binding of 17H6 to several normal tissues of critical organs shows that 17H6 has little cancer specificity.

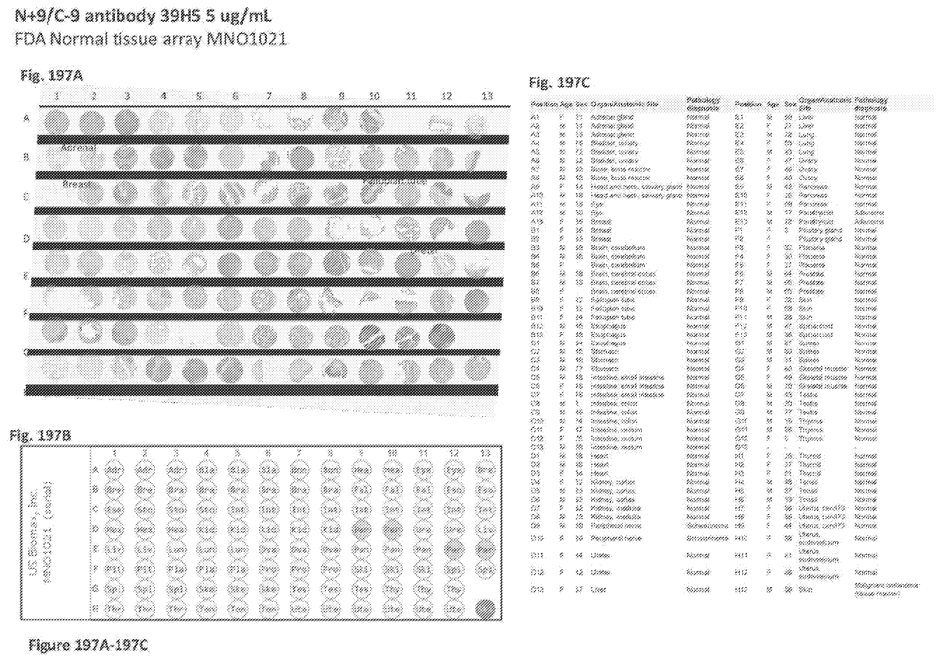

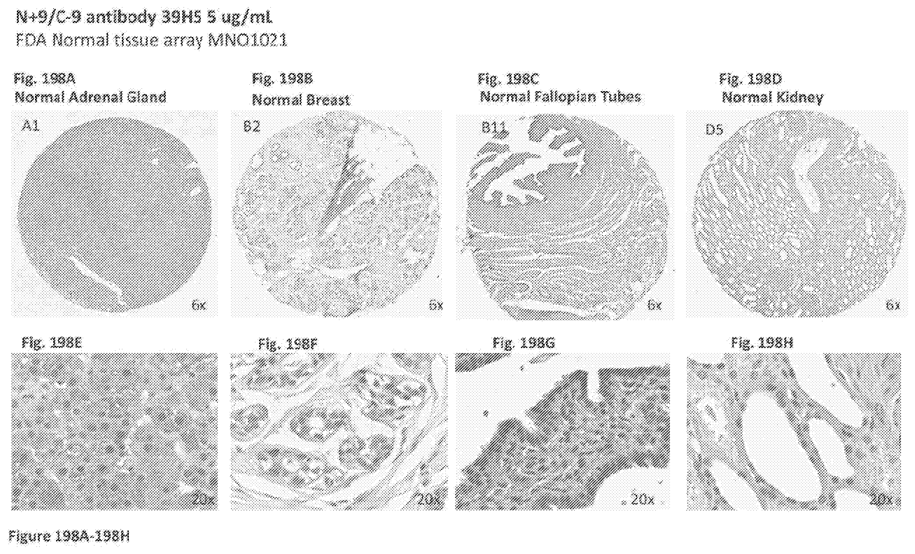

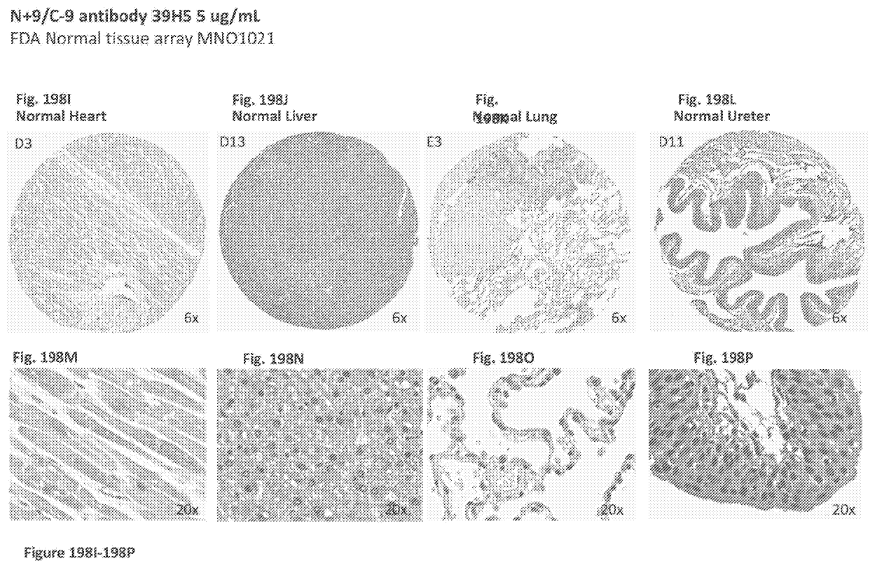

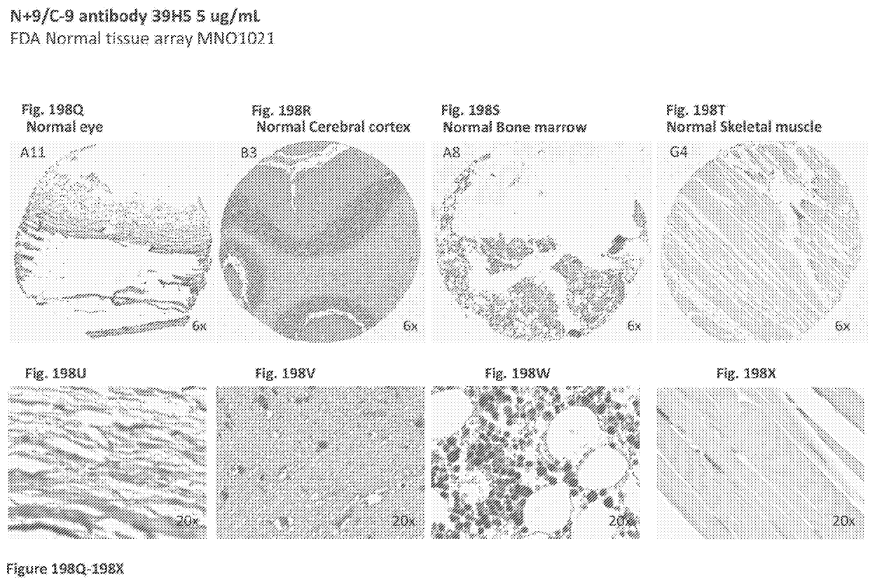

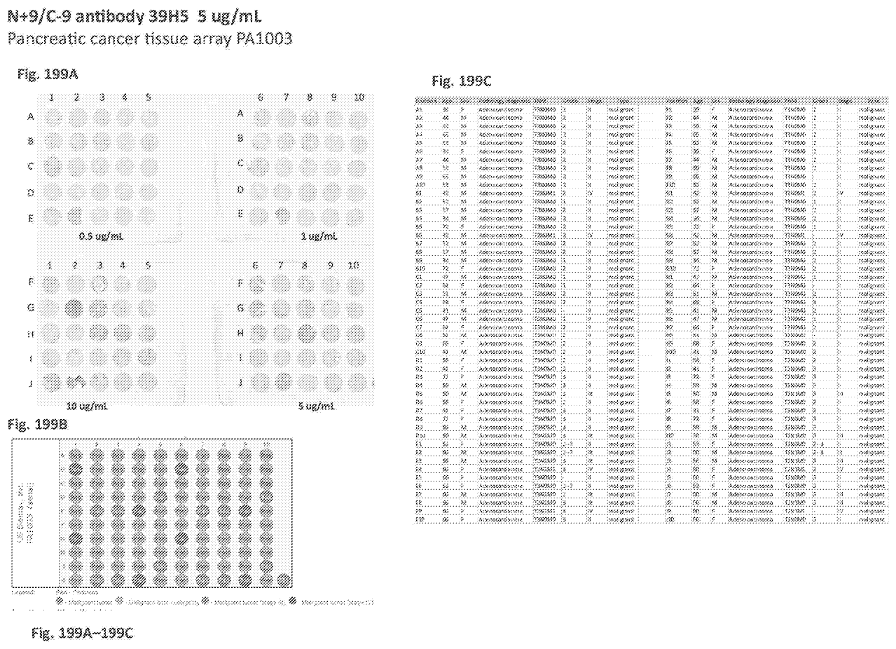

Antibody 39H5 is an N+9/C-9 antibody that binds weakly to the intact PSMGFR peptide but not significantly to any of the subset peptides. 39H5 may bind to the GTINVHDVET, which is the most distal part of the PSMGFR sequence. Examination of the entire normal tissue micro array and the magnified images (FIG. 197-198) shows that antibody 39H5 stains some normal tissues, including brain, heart, liver and bone marrow. 39H5 was used to probe a pancreatic cancer array, (FIG. 199-200). Although 39H5 stained a good percentage of the pancreatic cancer specimens, considering the normal tissues that 39H5 stained, 39H5 has little cancer specificity.

### Summary of FACS Analysis

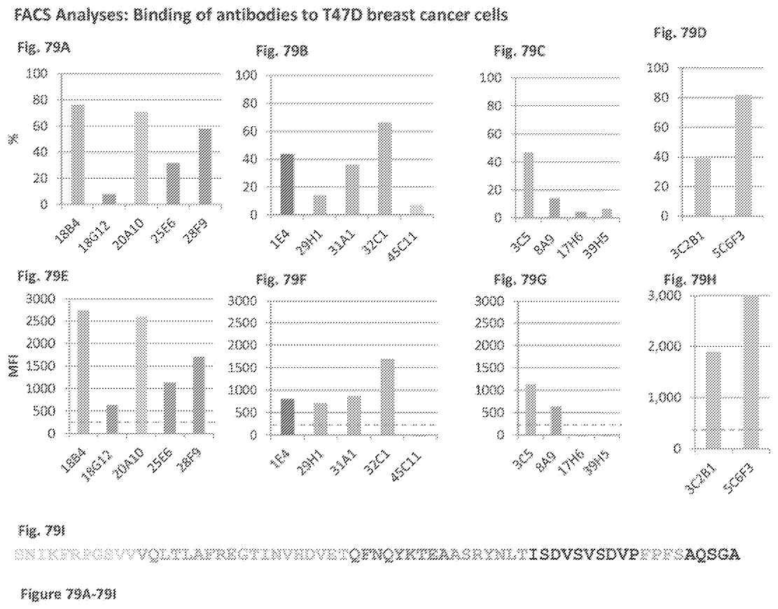

Determining the cancer specificity of antibodies using cell lines is difficult, as these cells were obtained from a single patient's tumor decades ago, and then propagated in culture for decades. Even if the patient's tumor was at one point heterogeneous, the decades of in vitro culture have essentially made the cell line a single cell clone. Antibodies of the invention were assayed by FACS to determine if they bound to MUC1 or MUC1* positive cancer cells but not MUC1 negative cells. The results of these experiments are shown in FIGS. 76-87. What is very clear is that antibodies that bind to epitopes of the MUC1 sequence that are outside of and N-terminal to PSMGFR sequence show no cancer specificity. Referring now to the readings of Mean Fluorescence Intensity (MFI) it appears that antibodies with cognate epitopes at the very N-terminus of the PSMGFR sequence, such as those that bind to an epitope within GTINVHDVET, show far less cancer specificity than the antibodies that recognize more C-terminal epitopes. For example, antibody MNC2 that will not bind to the C-10 peptide binds strongly to nearly every MUC1* positive cell line (FIG. 76-77). However, closer examination reveals that MNC2 binds lung cancer line NCI-H1975 much more strongly than NCI-H292. Similarly, MNC2 binds pancreatic cell line HPAF-II much better than Capan-1 or prostate cancer line DU145. PCR measurements show that the expression levels of cleavage enzymes varies greatly across a panel of cancer cell lines (FIG. 43 and FIG. 44). The fold of the MUC1* extra cellular domain can vary greatly depending on which cleavage enzyme clips it, which likely accounts for differences between cancer cell lines that a single antibody recognizes. This variation in antibody recognition of various cell lines, even within a cancer sub-type is apparent in the figures.

### Summary of IHC Data

IHC analysis of real tissues, including both normal and cancerous tissues, is more informative than the study of cultured cell lines, as is necessary in FACS analysis. Each antibody was first tested over a range of concentrations to determine optimal concentration. Antibody concentration was increased until the stroma also picked up stain, which indicates non-specific background binding. The optimal concentration for that particular antibody was then deemed to be just below the concentration at which the antibody stained the stroma.

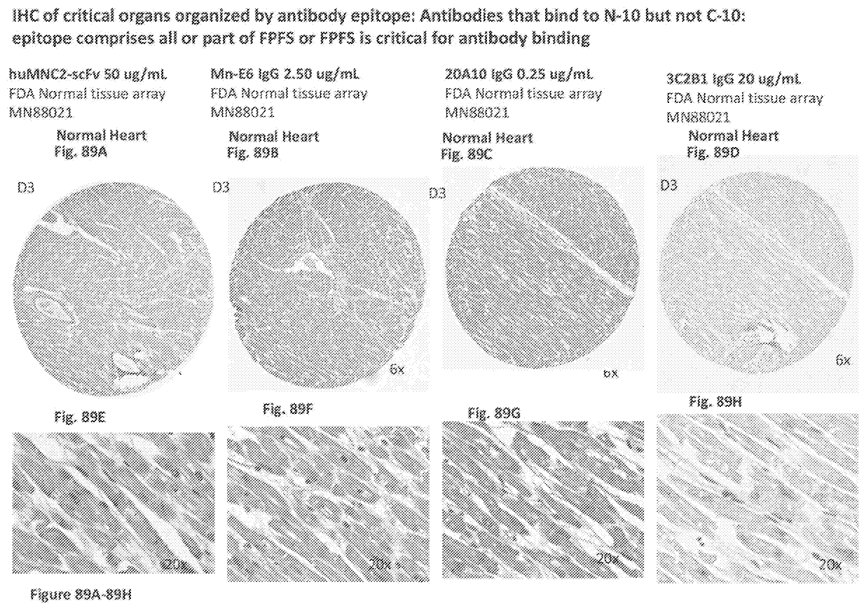

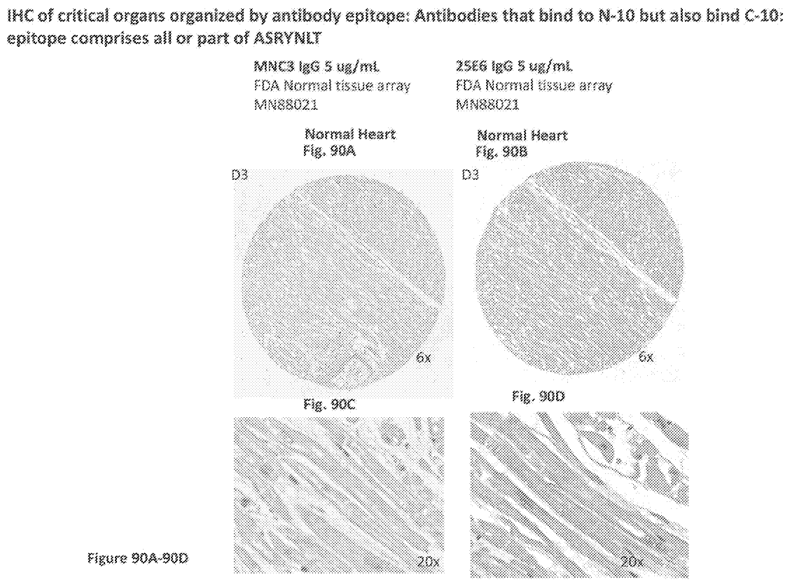

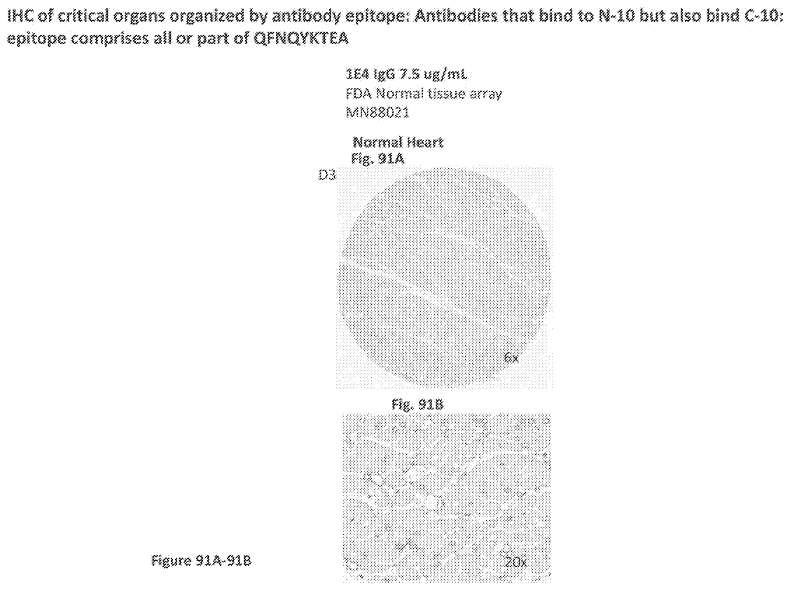

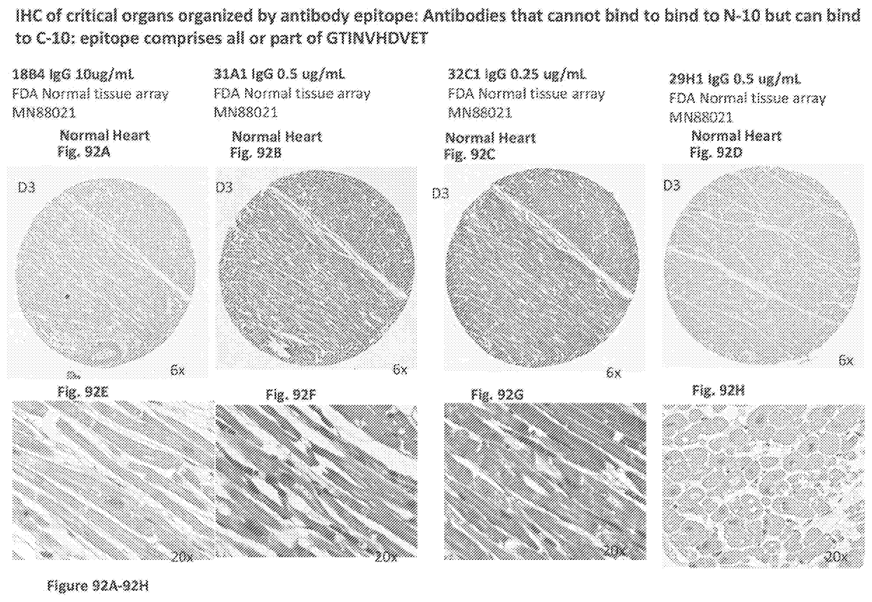

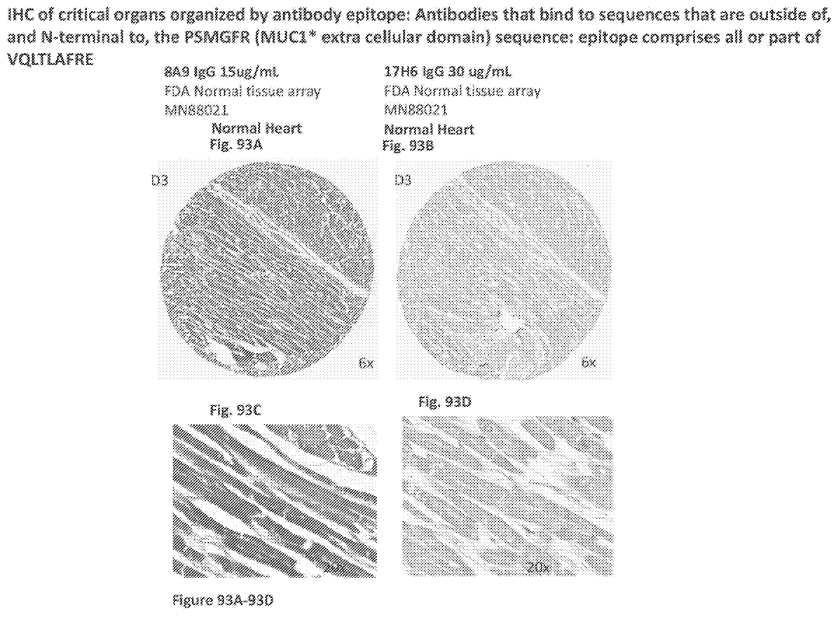

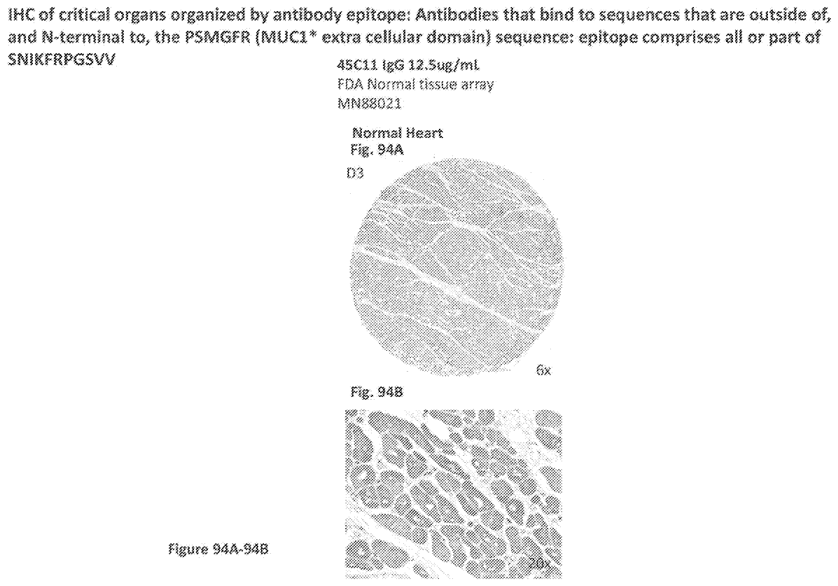

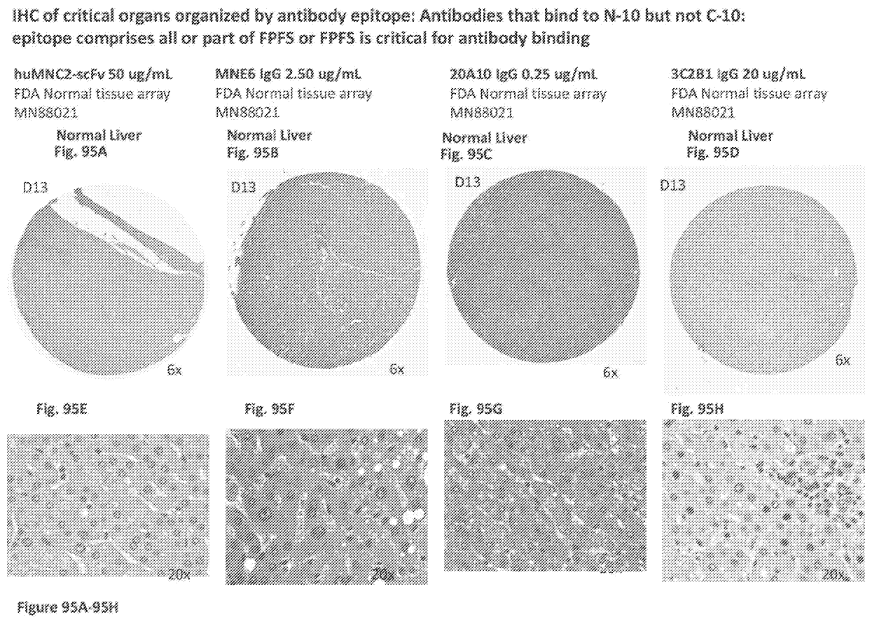

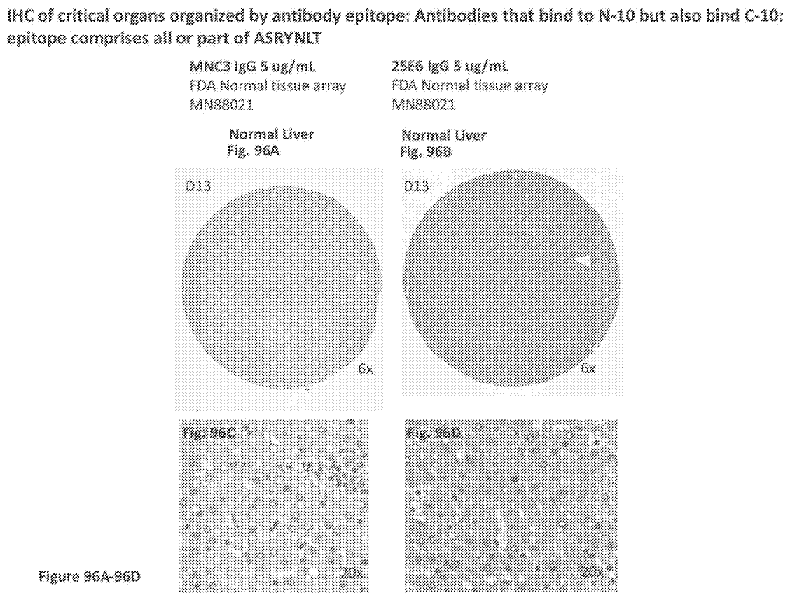

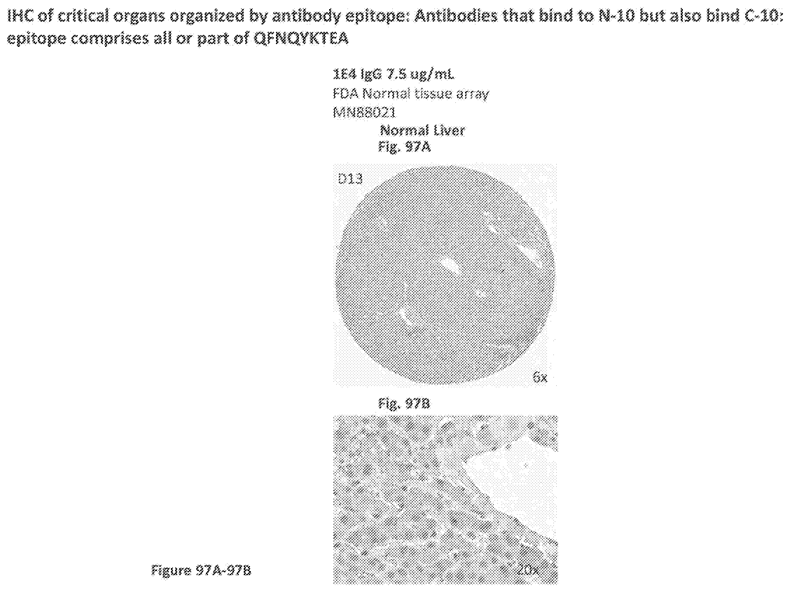

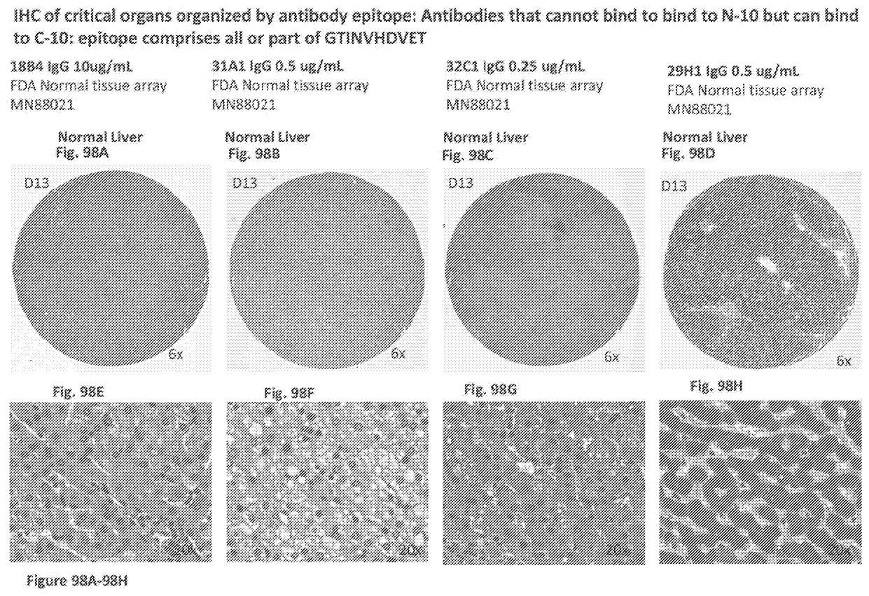

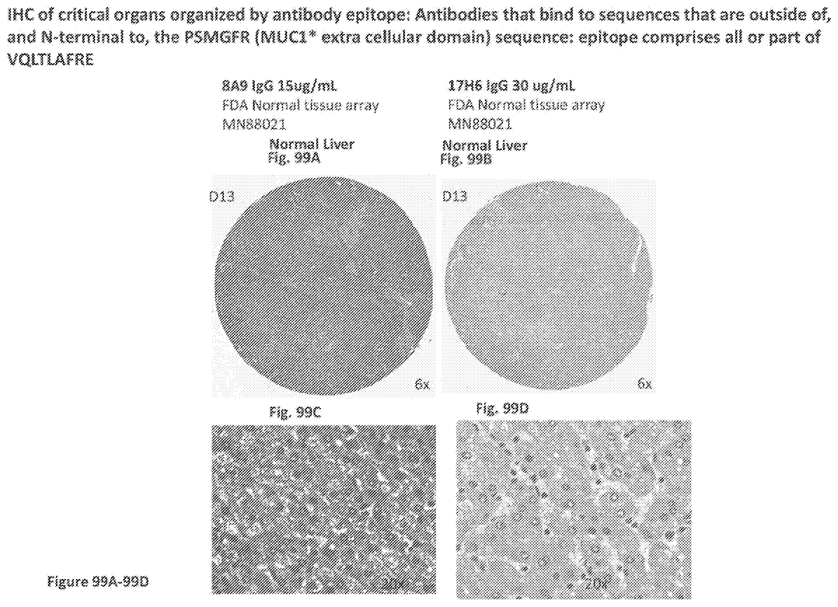

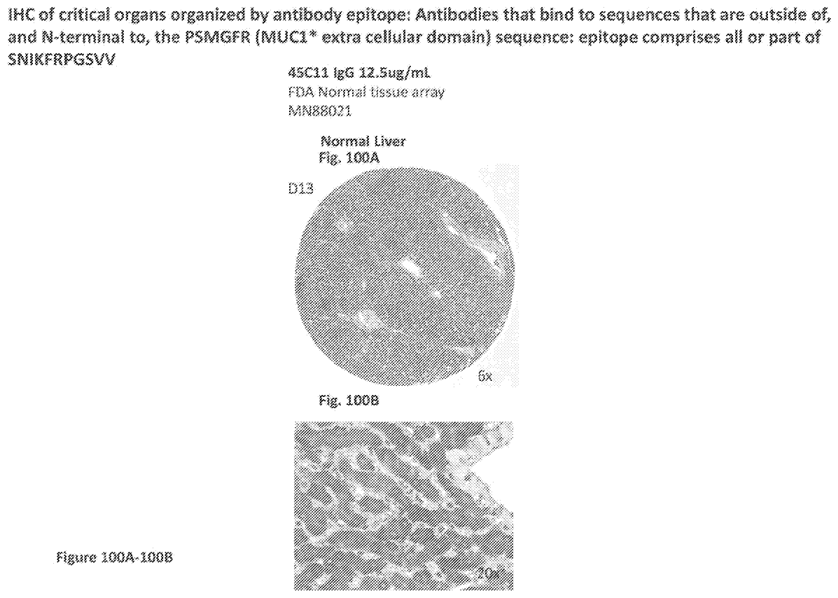

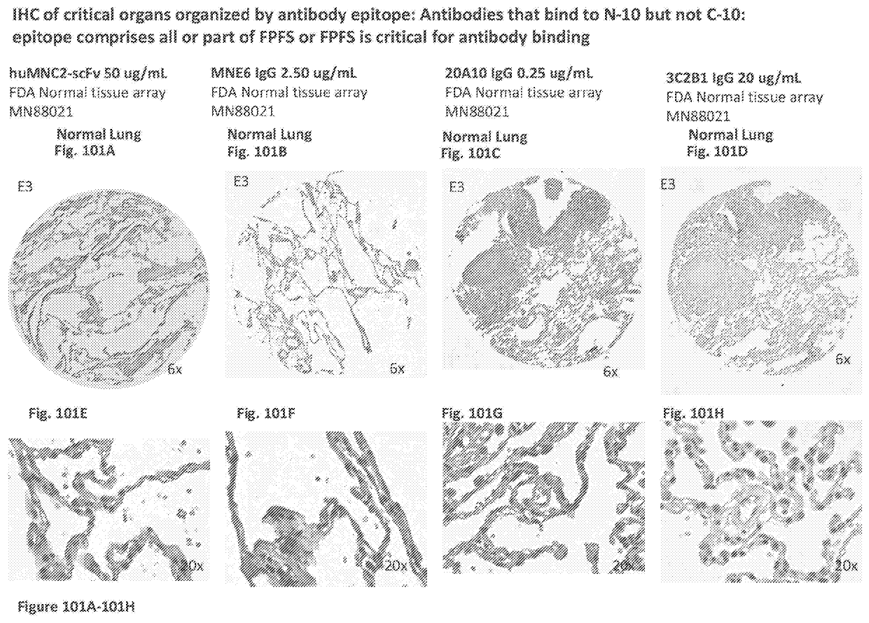

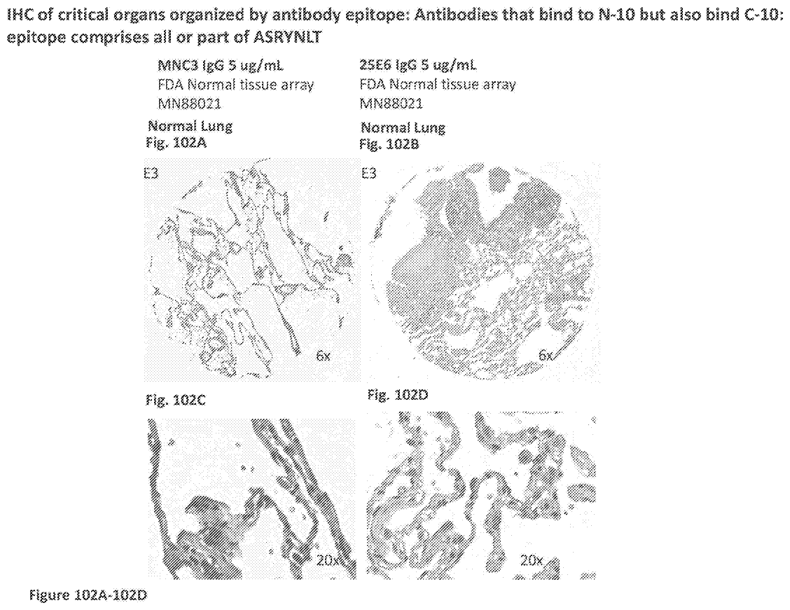

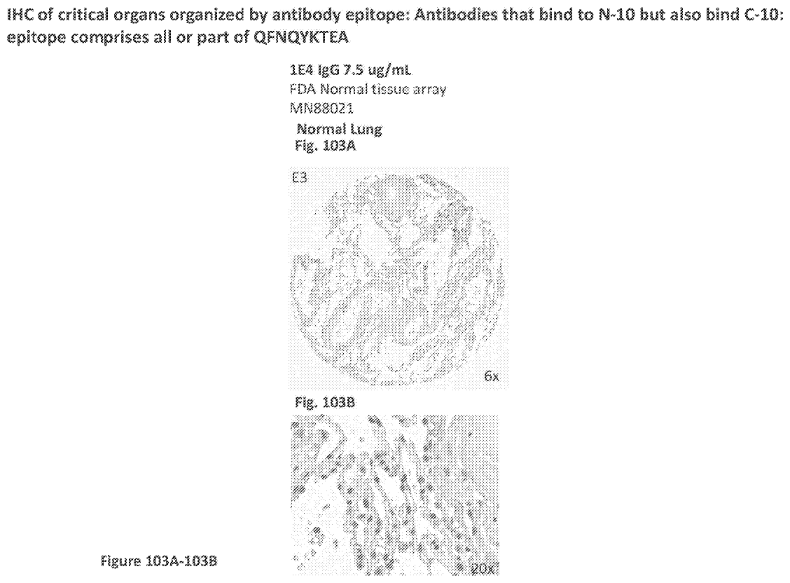

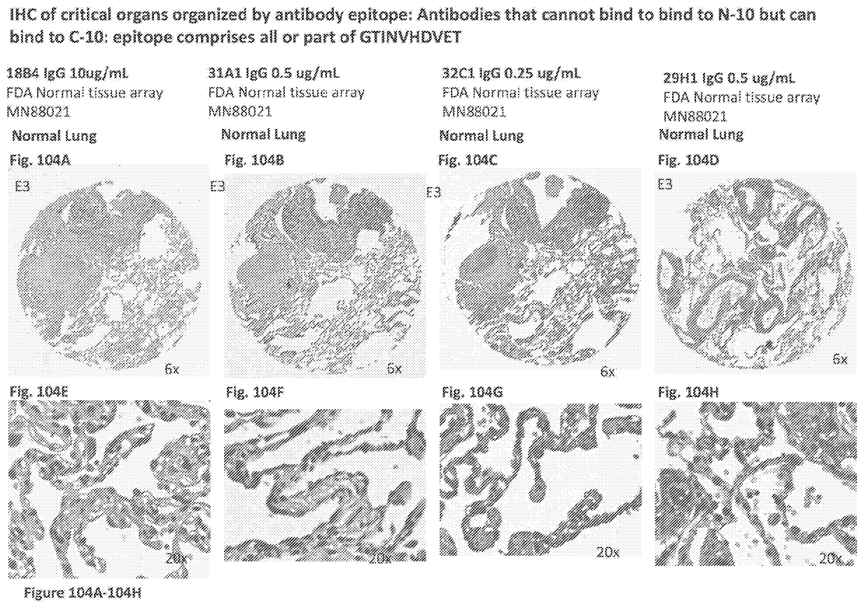

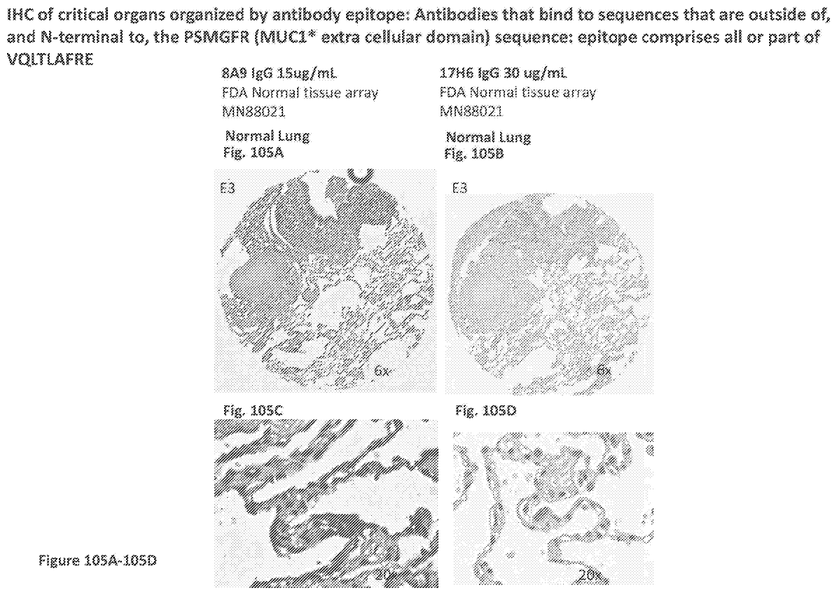

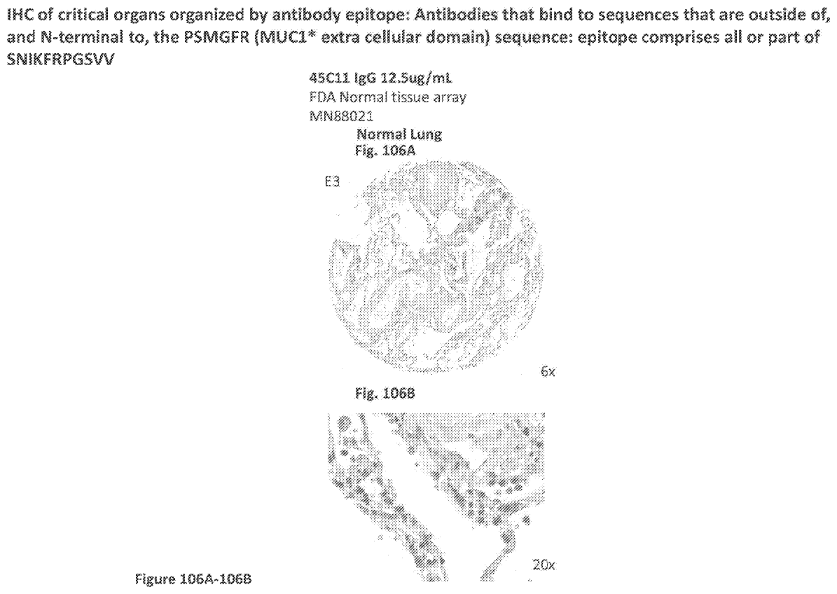

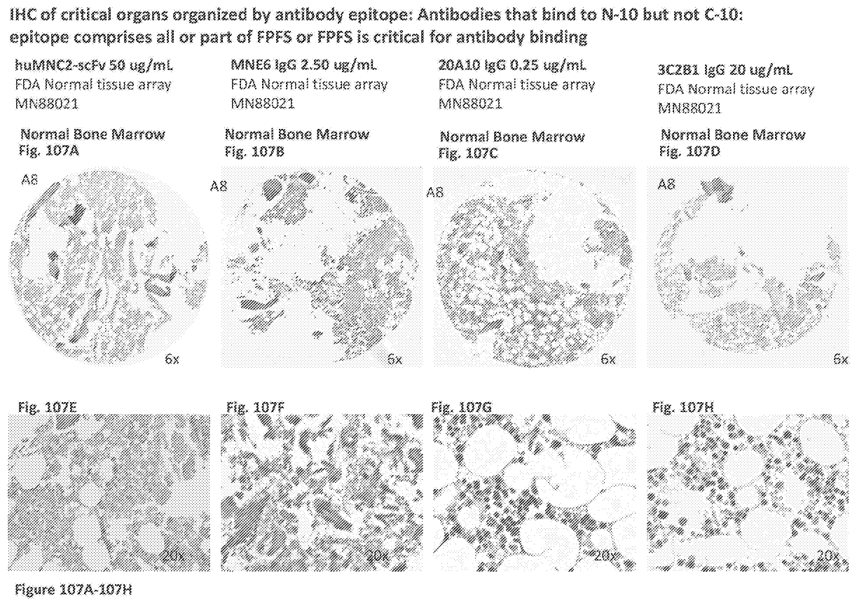

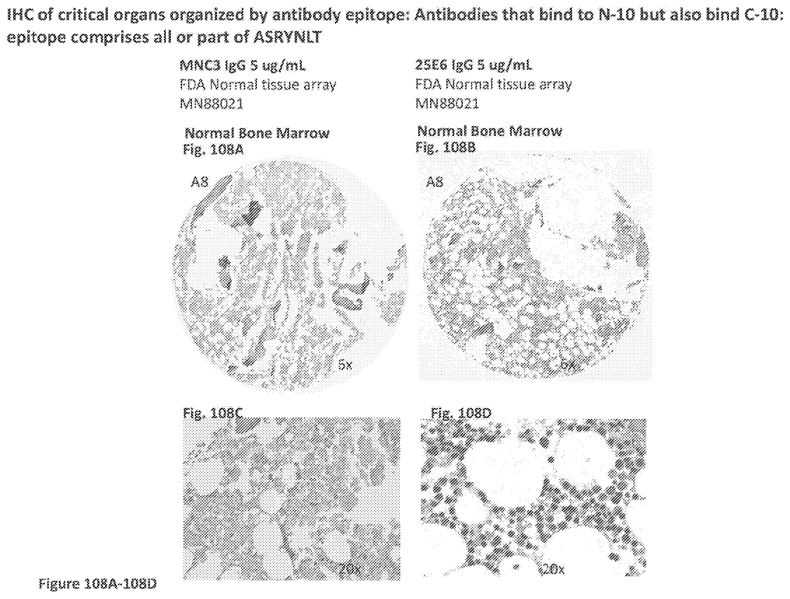

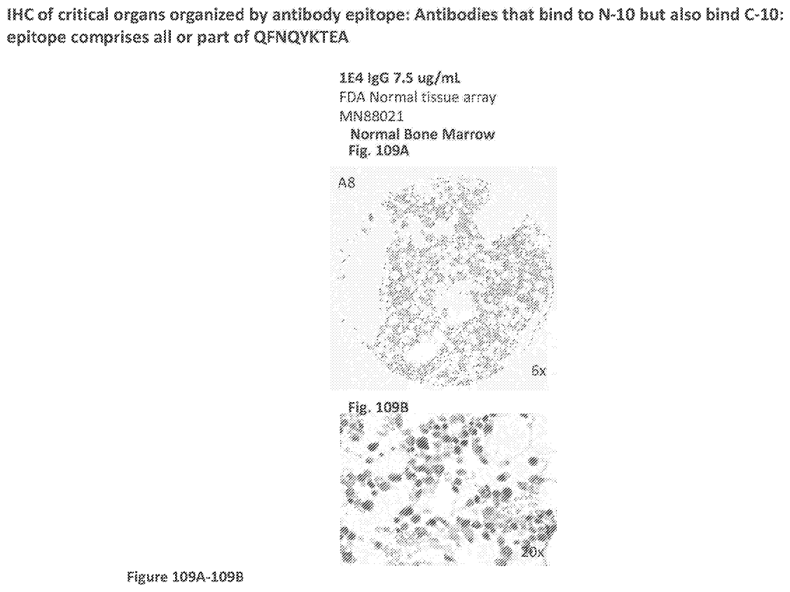

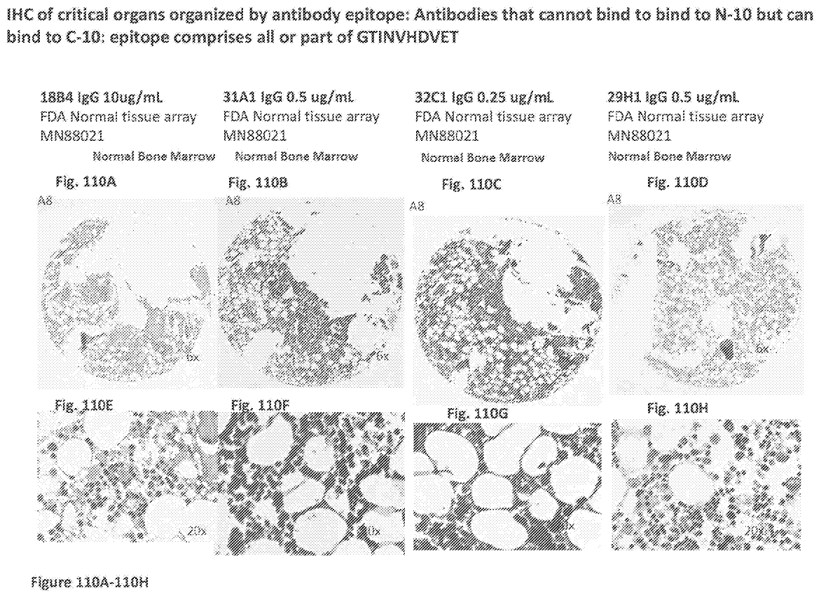

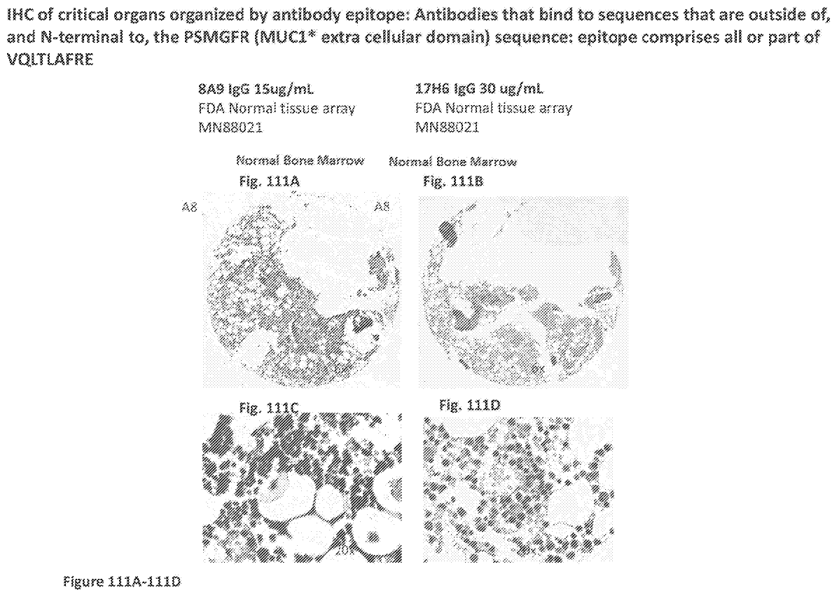

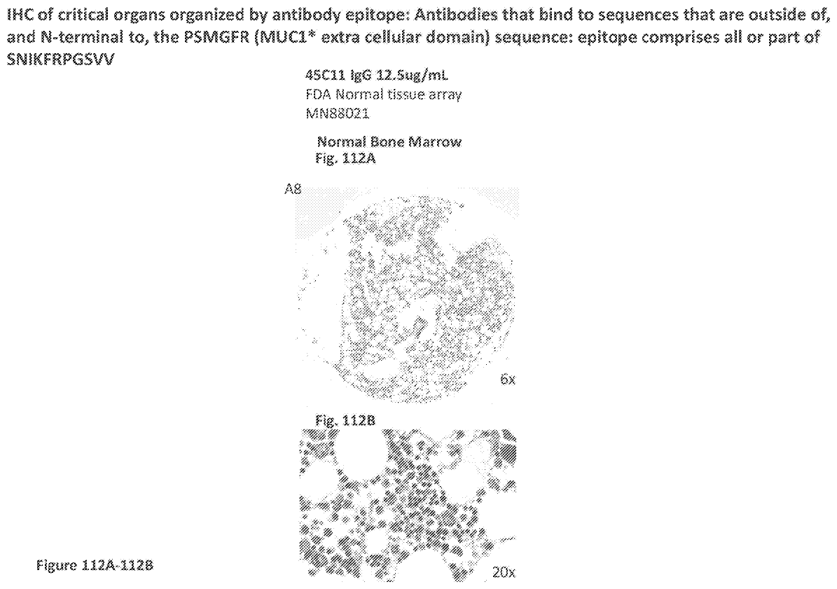

An overview of the IHC tissue studies is shown in FIG. 88-112. Here, we focused on the binding of antibodies to critical organ tissues, since binding to certain normal tissues would likely eliminate therapeutic use of that antibody. In these figures, the antibodies were grouped according to their cognate epitope. What is evident from the tissue studies is that the further the epitope is from the cell membrane, the more it binds to normal MUC1 on normal tissues. For example, binding to normal heart tissue by representative antibodies that recognize a specific epitope are shown in FIG. 88A-88L. As the figure illustrates, antibodies that bind to epitopes that are N-terminal to the PSMGFR peptide such as epitope within SNIKFRPGSVV or VQLTLAFRE show such strong binding to normal heart that they could not be used in therapeutics. In addition, antibodies that bind to the more N-terminal portion of PSMGFR, such as 29H1, also show binding to normal heart. The antibodies with the least binding to normal tissues and the strongest binding to cancerous tissues bind to epitopes within the FPFS or PFPFSAQSGA. Some antibodies that bind to epitopes within the ASRYNLT portion may also be suitable as therapeutics. These antibodies and others that recognize the same epitopes are desirable as anti-cancer therapeutics because they have a large therapeutic window, meaning that because of the low binding to normal tissues, and low side effects, patients can be dosed with antibody levels high enough to effectively kill the tumor cells. More detailed photographs of antibodies of the invention binding, or not binding, to other critical tissues are also shown. FIGS. 89-94 show magnified photographs of each antibody binding to normal heart tissue, where the antibodies have been categorized according to which epitope they bind. FIGS. 95-100 show magnified photographs of each antibody binding to normal liver tissue, where the antibodies have been categorized according to which epitope they bind. FIGS. 101-106 show magnified photographs of each antibody binding to normal lung tissue, where the antibodies have been categorized according to which epitope they bind. FIGS. 107-112 show magnified photographs of each antibody binding to normal bone marrow, where the antibodies have been categorized according to which epitope they bind.

The results of the IHC studies (FIG. 88-FIG. 200) are summarized in Table 3.

As can be clearly seen in the table, the further away from the cell membrane that the antibody binds, the more non-specific binding there is. Although these antibodies were generated by immunizing with the PSMGFR peptide, N+20/C-27 peptide or the C+9/C-9 peptide, some of the antibodies generated by immunizing with an extended peptide still bind within the PSMGFR sequence, see FIGS. 63-69 for the details of epitope binding for each antibody. Some binding to normal tissues can be tolerated if the antibody is incorporated into an appropriate therapeutic format. For example, cellular therapies, such as CAR T, are carried by the blood and meet with physiological barriers including lamina propia and blood-brain barrier that limits the cell's access to luminal edge of ducts and glands. Other antibodies that bind much more strongly to cancerous tissues but do show some binding to normal tissues could also be safe and useful therapeutics if administered locally or if cancer-specificity is enhanced by incorporating into a bispecific antibody. However, widespread antibody binding to many normal organs or to essential organs for which there is no physical barrier could be lethal to the patient.

The most cancer-specific antibodies with little to no binding to normal tissues are MNC2, MNE6, 20A10, 3C2B1 and 25E6. An ideal antibody therapeutic is one that stains no normal tissues but robustly stains cancer cells. Unfortunately, cancer antigens are also expressed on normal tissues, so zero staining of normal tissue is not possible. The aim is to identify an antibody that binds much more strongly to tumor tissue than normal tissue and that either binds to non-critical normal tissues or binds to them in a way that would not be physiologically possible in an intact organ. For example, CAR T cells are carried by the blood and the lamina propia is a barrier to their getting to the luminal edge of a duct or gland. Similarly, the blood brain barrier prevents the passage of large molecules like antibodies from the blood into the brain. The usefulness of an antibody as a therapeutic also depends on the format of the therapeutic. As mentioned, cell based therapies have natural barriers that prevent the CAR T cells from getting to some normal tissues. Antibody Drug Conjugate (ADC) based therapies sometimes depend on a local, cancer-specific molecule to activate the toxin attached to the antibody, minimizing the importance of whether or not a naked antibody binds to some normal tissue. In another example, antibodies and antibody-based therapeutics can be administered locally, including intraperitoneally, to maximize the effect on tumor cells while minimizing their effect on normal tissues. In yet another example, an antibody that is not purely cancer-specific can be made more cancer-specific if it is incorporated into a bispecific antibody where a first side of the molecule binds to a first cancer antigen and the second side of the molecule binds to a second antigen that may be a tissue specific antigen, another cancer specific antigen or even an antigen on a cell such as a T cell, which are called BiTES, bispecific T cell engagers. In yet another example, the less cancer-specific antibody can be incorporated into a cell-based therapy where its expression is induced only after the cell recognizes a tumor. In one aspect, a CAR T cell can express a first CAR that recognizes a first antigen which recognition induces expression of a second antibody, or CAR incorporating the second antibody. In one aspect the cell expresses a CAR directed by an antibody fragment that is cancer-specific and a second antibody or CAR expressing the second antibody is induced to be expressed in an NFAT inducible system. In one aspect the nucleic acids encoding the second antibody or second CAR are down stream of NFAT response elements. The NFAT inducible gene may be inserted into a Foxp3 enhancer or promoter.

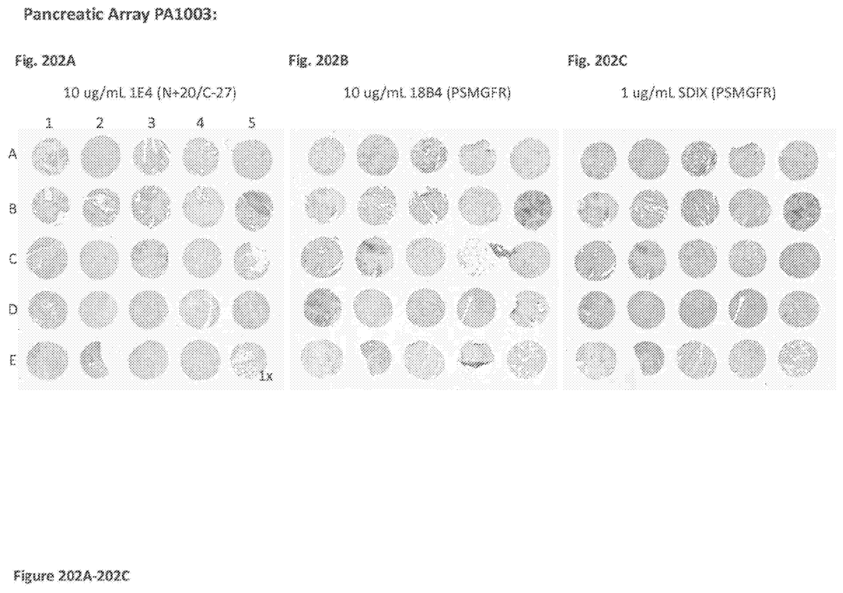

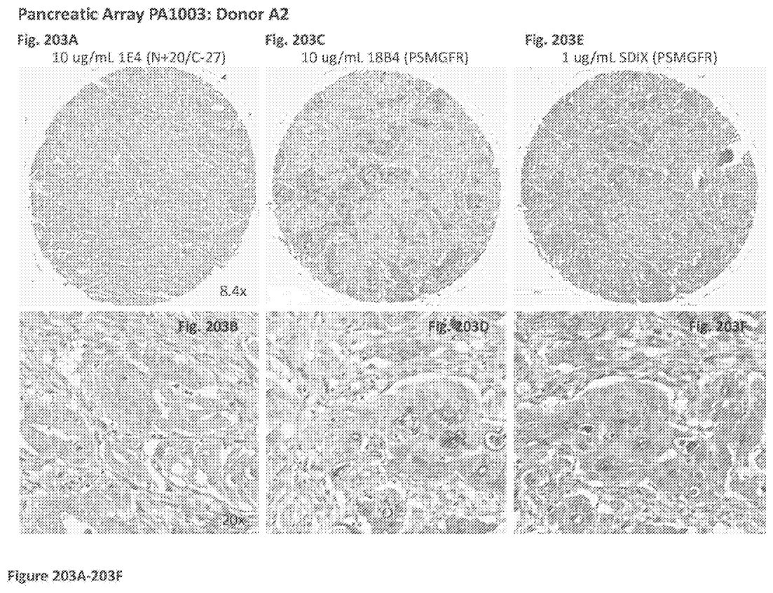

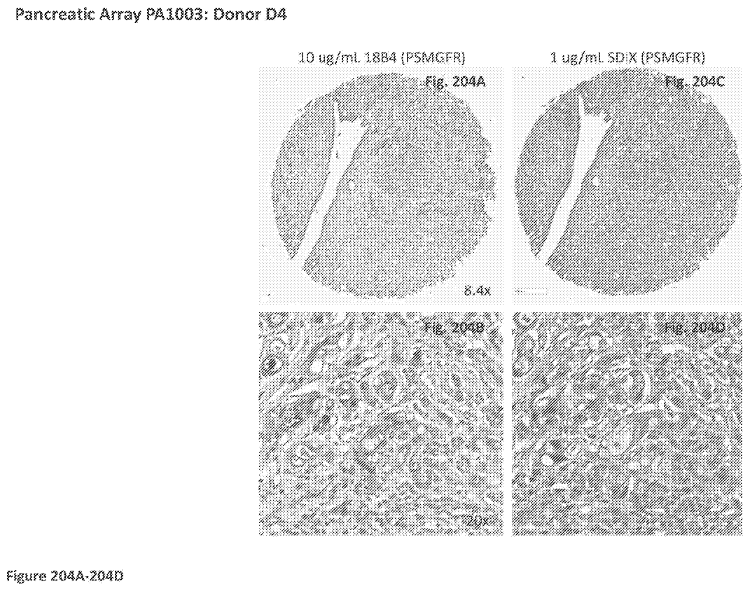

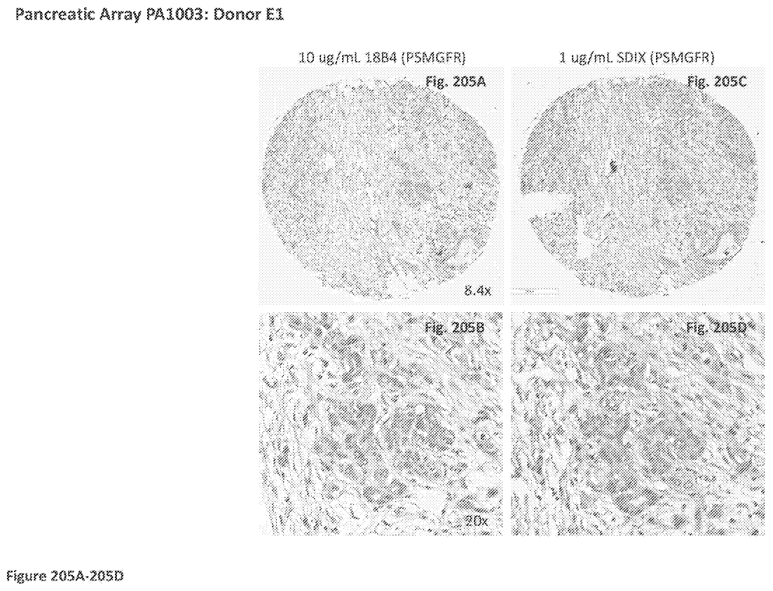

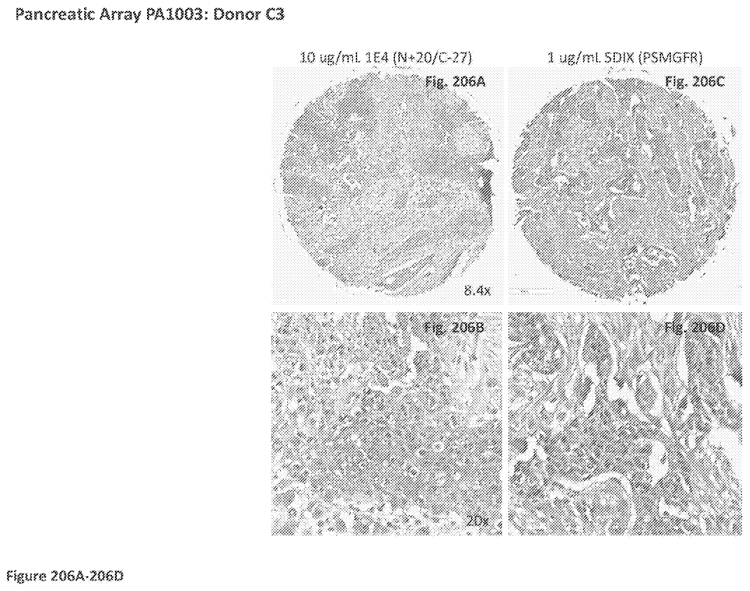

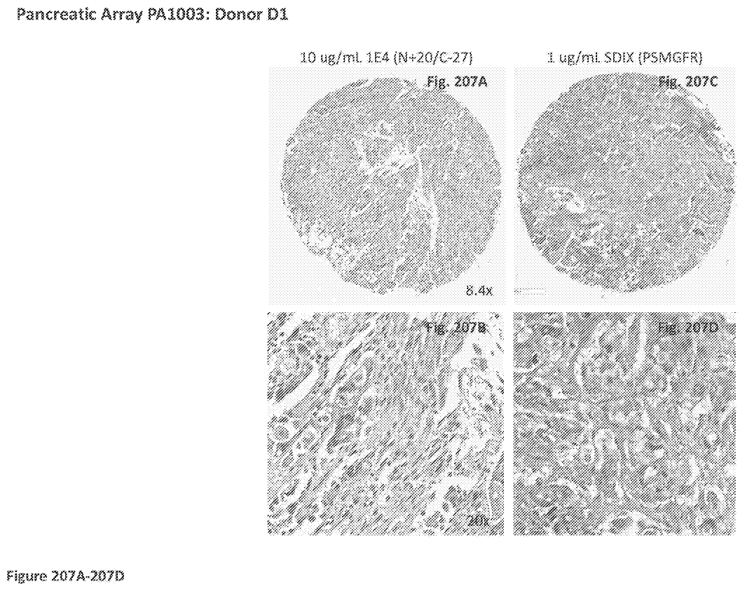

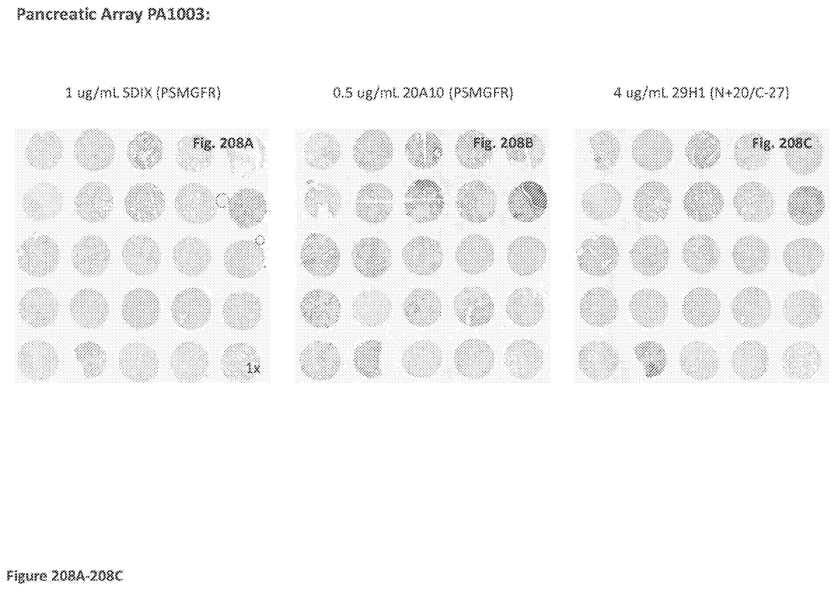

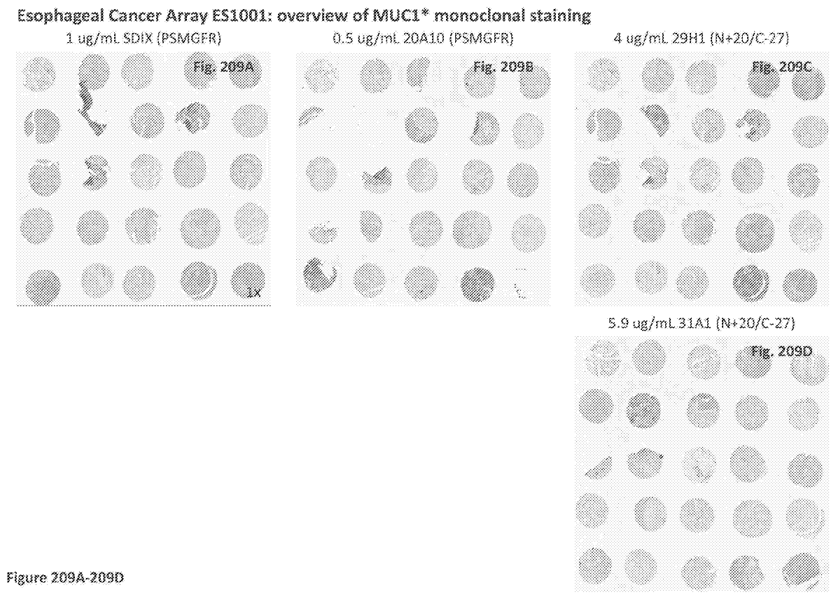

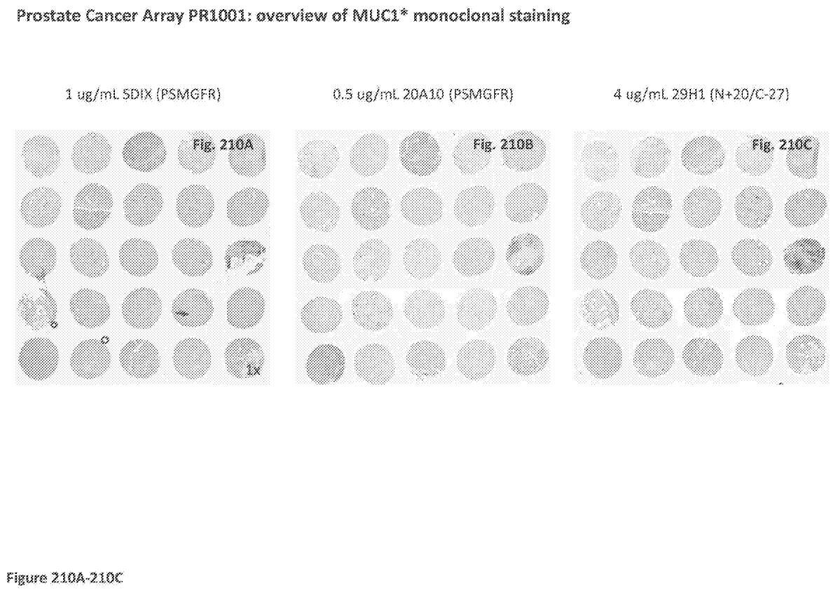

FIG. 202 shows photographs of pancreatic cancer tissues, each from a different patient. As can be seen, the staining pattern of 1E4 is very different from that of 18B4 and the polyclonal antibody SDIX. 18B4 and SDIX antibodies were generated by immunizing animals with the same peptide (PSMGFR), while the 1E4 antibody was generated from immunization with a different peptide (N+20). FIGS. 203-207 show magnified images of selected tissues from this array to highlight the differences between these antibodies. FIG. 208 compares the staining of polyclonal antibody SDIX to monoclonal antibody 20A10, which were both generated from immunization with the PSMGFR peptide. Also shown is the difference in staining pattern for antibody 29H1 which was generated by immunization with an N+20 peptide. Although the antibody staining is lighter, antibody 29H1 recognizes more pancreatic cancer tissue specimens than the SDIX polyclonal or 20A10. FIG. 209 shows that esophageal cancers are belier recognized by antibodies that bind to a MUC1* peptide with an extended N-terminus, such as antibody 29H1 and antibody 31A1. Similarly, FIG. 210 shows that prostate cancers are belier recognized by antibodies that bind to a MUC1* peptide with an extended N-terminus, such as antibody 29H1.

Below Table 4 shows a summary of the test criteria to determine the cancer-specificity of the various monoclonal antibodies.

TABLE 4
Cancer-Specificity Test Criteria

| | | | | | | | | |
|---|---|---|---|---|---|---|---|---|
| | | | | 3 | 4 | 5 | | |
| | | | 2 | Displaces | Does not | Recognizes | 6 | 7 |
| | | 1 | Does | NME7_{AB} | recognize | MUC1 after | Cancer | Cancer |
| | Binds | Binds | not bind | from | linear | cleavage | selective | selective |
| mAb Name | PSMGFR | N-10 | C-10 | MUC1* | epitope | by MMP9 | by FACS | by IHC |

SNIKFRPGSVVVQLTLAFREGTINVHDVETQFNQYKTEAASRYNLTISDVSVSDVPFPFSAQSGA

| | | | | | | | | |
|---|---|---|---|---|---|---|---|---|
| FPFS | | | | | | | | |
| MNC2 | [symbol] | [symbol] | [symbol] | [symbol] | [symbol] | [symbol] | [symbol] | [symbol] [symbol] [symbol] |
| MNE6 | [symbol] | [symbol] | [symbol] | [symbol] | [symbol] | [symbol] | [symbol] | [symbol] [symbol] [symbol] |
| 20A10 | [symbol] | [symbol] | [symbol] | [symbol] | [symbol] | [symbol] | [symbol] | [symbol] [symbol] [symbol] |
| 3C2B1 | [symbol] | [symbol] | [symbol] | [symbol] | [symbol] | [symbol] | [symbol] | [symbol] [symbol] [symbol] |
| SVSDV | | | | | | | | |
| 5C6F3 | [symbol] | [symbol] | ~ [symbol] | [symbol] | [symbol] | [symbol] | [symbol] | [symbol] [symbol] |
| ASRYNLT | | | | | | | | |
| 25E6 | [symbol] | [symbol] | [symbol] | [symbol] | [symbol] | [symbol] | [symbol] | [symbol] [symbol] |
| MNC3 | [symbol] | ~√ | [symbol] | [symbol] | [symbol] | [symbol] | ND | [symbol] |
| 18G12 | [symbol] | [symbol] | [symbol] | [symbol] | [symbol] | [symbol] | ~√ | [symbol] |
| 28F9 | [symbol] | [symbol] | [symbol] | [symbol] | [symbol] | [symbol] | ~√ | [symbol] |
| QFNQYKTEA | | | | | | | | |
| 1E4 | [symbol] | [symbol] | [symbol] | [symbol] | [symbol] | [symbol] | ~√ | [symbol] |
| GTINVHDVET | | | | | | | | |
| 18B4 | [symbol] | [symbol] | [symbol] | ~√ | [symbol] | [symbol] | [symbol] | [symbol] |
| 29H1 | [symbol] | [symbol] | [symbol] | ~√ | [symbol] | [symbol] | [symbol] | [symbol] |
| 31A1 | [symbol] | [symbol] | [symbol] | [symbol] | [symbol] | [symbol] | [symbol] | [symbol] |
| 32C1 | [symbol] | [symbol] | [symbol] | ~√ | [symbol] | [symbol] | [symbol] | [symbol] |
| 39H5 | [symbol] | [symbol] | [symbol] | ~√ | [symbol] | [symbol] | [symbol] | [symbol] |
| 3C5 | [symbol] | [symbol] | [symbol] | [symbol] | [symbol] | [symbol] | [symbol] | [symbol] [symbol] |
| VQLTLAFRE | | | | | | | | |
| 8A9 | [symbol] | [symbol] | [symbol] | [symbol] | [symbol] | [symbol] | [symbol] | [symbol] [symbol] |
| 17H6 | [symbol] | [symbol] | [symbol] | [symbol] | [symbol] | [symbol] | [symbol] | [symbol] |
| SNIKFRPGSVV | | | | | | | | |
| 45C11 | [symbol] | [symbol] | [symbol] | [symbol] | [symbol] | [symbol] | [symbol] | [symbol] [symbol] [symbol] |

To summarize, we found that antibodies that bound to sequences that are N-terminal to the PSMGFR sequence had no cancer-specificity. Further, the closer to the cell membrane that the antibody binds, the more cancer-specific is the antibody. More importantly, test criteria 1-4 or even 1-5 provide a set of rapid, multiplexed and inexpensive tests that can be performed on hundreds or thousands of impure hybridoma clone supernatants to identify antibodies that are highly selective for cancer-specific forms of MUC1*.

### Satisfies Test Criteria

In a preferred embodiment an antibody is chosen for the treatment, prevention or diagnosis of a MUC1* positive cancer based on satisfying four (4) of the seven (7) criteria set out in Table 4. In a more preferred embodiment an antibody is chosen for the treatment, prevention or diagnosis of cancer based on satisfying five (5) of the seven (7) criteria set out in Table 4. In a yet more preferred embodiment an antibody is chosen for the treatment, prevention or diagnosis of cancer based on satisfying six (6) of the seven (7) criteria set out in Table 4. In a more preferred embodiment an antibody is chosen for the treatment, prevention or diagnosis of cancer based on satisfying all seven (7) of the criteria set out in Table 4. An antibody selected for suitability as a treatment for MUC1* positive cancers by virtue of satisfying four or more of the criteria set out in Table 4 can be incorporated in part or in whole into several therapeutic formats. In one aspect of the invention, the antibody or antibody fragment is incorporated into a CAR that is then expressed in an immune cell, which may be a T cell, then administered to a patient who has been diagnosed with or is at risk of developing a MUC1* positive cancer. In another aspect of the invention, a fragment of the antibody which may be an scFv, is incorporated into a BiTE, then administered to a patient who has been diagnosed with or is at risk of developing a MUC1* positive cancer. In yet another aspect of the invention, a fragment of the antibody which may be an scFv, is incorporated into a bi-specific antibody, then administered to a patient who has been diagnosed with or is at risk of developing a MUC1* positive cancer. In yet another aspect of the invention, the antibody or antibody fragment, is conjugated to a toxin or an ADC, antibody drug conjugate, then administered to a patient who has been diagnosed with or is at risk of developing a MUC1* positive cancer.

### Bind to N-10

We have demonstrated that a MUC1 transmembrane protein, devoid of tandem repeats and having an extra cellular domain of 45 amino acids of PSMGFR sequence, is sufficient to function as a growth factor receptor and confers oncogenic characteristics to the cell (Mahanta et al 2008). Antibodies that bind to the PSMGFR peptide or portion of a transmembrane MUC1 cleavage product can be cancer specific but may also bind to stem or progenitor cells. Antibodies that bind to the N-10 peptide are more cancer-specific. In a preferred embodiment an antibody is chosen for the treatment, prevention or diagnosis of cancer based on the ability of the antibody to bind to the N-10 peptide.

### Do not Bind to C-10

We have demonstrated that the MUC1 extra cellular domain contains an ectopic binding site that is only exposed if the tandem repeat domain is missing, which can occur as a consequence of alternative splice variant or cleavage and release of the extra cellular domain. Cancer-specific antibodies MNC2 and MNE6 will not bind to full-length MUC1, but do bind to the remaining portion when MUC1 is cleaved and the tandem repeat domain is shed. MNC2 and MNE6 will bind to a MUC1*-like protein if it is devoid of tandem repeats, for example if a MUC1 negative cell is transfected or transduced with an engineered MUC1 that is devoid of tandem repeats, especially if extra cellular domain comprises the PSMGFR. Thus, the ectopic site to which MNC2 and MNE6 bind is unmasked when tandem repeat domain is missing or removed. Both MNC2 and MNE6 require the 10 membrane proximal amino acids of a MUC1* extra cellular domain for binding; they do not bind to the C-10 peptide. That means that the ectopic binding site for MNC2 and MNE6 is within or contains all or part of the 10 C-terminal amino acids of the PSMGFR: PFPFSAQSGA. In a preferred embodiment an antibody is chosen for the treatment, prevention or diagnosis of cancer based on the inability of the antibody to bind to the C-10 peptide. In a preferred embodiment an antibody is chosen for the treatment, prevention or diagnosis of cancer based on the ability of the antibody to bind to the N-10 peptide and the inability of the antibody to bind to the C-10 peptide.

Compete with NME7_{AB} or NME7-X1 for Binding to MUC1* Positive Cell, PSMGFR Peptide or N-10 Peptide

We have demonstrated that cancer-specific antibodies MNC2 and MNE6 bind to an ectopic epitope that comprises all or part of the 10 C-terminal amino acids of the PSMGFR peptide: PFPFSAQSGA. We have shown that growth factors, dimeric NME1 and NME7_{AB}, also bind to an ectopic epitope that comprises all or part of the 10 C-terminal amino acids of the PSMGFR peptide. MNC2 and MNE6 compete with dimeric NME1 or NME7_{AB} for binding to the PSMGFR peptide and the N-10 peptide. In a preferred embodiment an antibody is chosen for the treatment, prevention or diagnosis of cancer based on the ability of the antibody to disrupt the binding of NME1, NME7_{AB}, or NME7-X1 to the PSMGFR peptide, the N-10 peptide, or to the surface of a MUC1* positive cancer cell.

Recognize a Conformational Epitope Rather than a Linear Epitope

Antibodies that are cancer-specific will be chosen based on their ability to bind to a MUC1 that is devoid of tandem repeats and for their inability to bind to full-length MUC1. Most often, MUC1* is generated when MUC1 is cleaved by a cleavage enzyme and the tandem repeat domain is released from the cell surface. Cleavage and release of the tandem repeat domain may also unmask portions of MUC1*-like cleavage products that exist on normal tissues. However, antibodies that recognize a conformation, rather than a linear epitope, are more selective. Antibodies that recognize a conformational epitope rather than a linear epitope can be identified by a variety of means. In particular, antibodies that recognize a conformational epitope will not work in a denaturing Western blot assay. In a preferred embodiment an antibody is chosen for the treatment, prevention or diagnosis of cancer based on the ability of the antibody to recognize a conformational epitope.

### Recognize a MUC1* Generated by Cleavage by MMP9 or Other Tumor-Associated Cleavage Enzyme

The fold, or conformation, of the MUC1* truncated extra cellular domain differs depending on which enzyme cleaves MUC1. Cleaved MUC1* or MUC1*-like cleavage products can function as growth factor receptors on normal healthy tissues. More than one cleavage enzyme is able to cleave MUC1 to a MUC1*-like form. Cleavage by first enzyme may produce a conformation or a fold that is not the same as that produced by cleavage by a second enzyme. Support for this can be found in this application and is illustrated in FIGS. 39-41. These figures show that although a polyclonal antibody that binds to PSMGFR recognizes a cleaved MUC1 on hematopoietic stem cells, some monoclonal antibodies that bind to the PSMGFR peptide can bind to this MUC1*-like form on hematopoietic stem cells while others cannot. For example, MNC3 readily recognizes this cleaved form of MUC1 on hematopoietic stem cells, but MNC2 and MNE6 do not. We know that MNC2 and MNE6 recognize a MUC1* that is produced by cleavage by MMP9 but not when it is cleaved by MMP2. MNC2 and MNE6 are cancer-specific while MNC3 is not, as it recognizes stem and progenitor cells. We also know that MMP9 is overexpressed in cancers. Bone marrow, where hematopoietic stem cells are made expresses nearly 2,500-times more MMP2 than MMP9 (FIG. 65). MMP14 is another enzyme that cleaves MUC1 to a MUC1* growth factor receptor form (FIG. 38). In one aspect of the invention, an antibody is chosen for the treatment, prevention or diagnosis of cancer based on the ability of the antibody to recognize a MUC1 cleavage product generated when MUC1 is cleaved by MMP14. In a preferred embodiment an antibody is chosen for the treatment, prevention or diagnosis of cancer based on the ability of the antibody to recognize a MUC1 cleavage product generated when MUC1 is cleaved by MMP9. In a preferred embodiment an antibody is chosen for the treatment, prevention or diagnosis of cancer based on the ability of the antibody to recognize a MUC1 cleavage product generated when MUC1 is cleaved by MMP9 and also recognizes a conformational epitope.

Binds to Cancer Cells More than Normal Cells

A traditional approach to identifying antibodies that are cancer-specific involves testing a panel of antibodies against a panel of different cancer cell lines and determining, by FACS, IF, immunoprecipitation or other method, if the antibody binds to cancer cells. Although this approach is traditional, it is sequential and time-consuming, and thus limits the analysis of large numbers of monoclonal antibody clones, which is required to find an ideal antibody suitable for cancer therapeutic or diagnostic. In addition, there are no real normal cell lines and the selection of normal primary cells is limited. The selection criteria presented above provide a rapid, multiplexed method for identifying monoclonal antibody clones that are specific for MUC1* positive cancers. For many of the selection criteria, hybridoma supernatants can be used. This provides a huge advantage over state of the art methods for identifying antibodies that are specific for MUC1* positive cancers. The ability to select antibodies from assay performed using the impure hybridoma supernatants means that much of the selection can be done on hundreds or thousands of clones rapidly and at very little cost. Methods such as FACS analysis ans IHC tissue studies require the use of purified antibodies which limits the number of clones that can be tested to tens, not even hundreds.

However, selecting an antibody based on its ability to bind to cancer cells, or a cancer cell type or to a cell engineered to express a certain antigen is important for antibody selection. In a preferred embodiment an antibody is chosen for the treatment, prevention or diagnosis of cancer based on the ability of the antibody to bind to MUC1* positive cancer cells.

Binds to Tumor Tissue More than Normal Tissue

Immunohistochemistry, IHC, tissue studies of cancerous versus normal tissues is a more stringent test of the cancer specificity of antibodies than FACS analysis. Cancer cell lines are a single cell from a single patient that have been expanded in a lab for decades and are not representative of a cross section of the human population. Further, analysis of cell lines is blind to the heterogeneity of actual tumors. Tissue studies require purified antibody, are very expensive, time-consuming and require a skilled pathologist to analyze each stained tissue specimen. However, antibody staining of tissues from normal tissues versus cancerous tissues can reveal which antibodies cannot be used as therapeutics or diagnostics because of their cross-reactivity with normal tissues. Our systematic studies of numerous antibodies with thousands of human normal tissues or cancerous tissues, across several cancer sub-types showed that antibodies that bind to N-10, not C-10, disrupt the binding of NME1 or NME7_{AB}, or NME7-X1 to the PSMGFR peptide, the N-10 peptide, or to the surface of a MUC1* positive cancer cell, recognize a conformational epitope, and recognize a conformational epitope created by cleavage by MMP9 are the most cancer-specific.

In a preferred embodiment an antibody is chosen for the treatment, prevention or diagnosis of cancer based on the ability of the antibody to bind to MUC1* positive tumor tissue at least 2-times more than it binds to normal tissues. In a preferred embodiment an antibody is chosen for the treatment, prevention or diagnosis of cancer based on the ability of the antibody to bind to MUC1* positive tumor tissue at least 5-times more than it binds to normal tissues. In a preferred embodiment an antibody is chosen for the treatment, prevention or diagnosis of cancer based on the ability of the antibody to bind to MUC1* positive tumor tissue at least 10-times more than it binds to normal tissues.

Antibodies that Bind to Refined Epitopes

In a preferred embodiment, an antibody, or fragments thereof, that binds to a peptide comprising the sequence QFNQYKTEAASRYNLTISDVSVSDVPFPFSAQSGA are incorporated into anti-cancer therapeutics or diagnostics for the diagnosis, treatment or prevention of a MUC1* positive cancer.

In a more preferred embodiment, an antibody, or fragments thereof, that binds to a peptide comprising the sequence ASRYNLTISDVSVSDVPFPFSAQSGA are incorporated into anti-cancer therapeutics or diagnostics for the diagnosis, treatment or prevention of a MUC1* positive cancer.

In a yet more preferred embodiment, an antibody, or fragments thereof, that binds to a peptide comprising the sequence SDVSVSDVPFPFSAQSGA are incorporated into anti-cancer therapeutics or diagnostics for the diagnosis, treatment or prevention of a MUC1* positive cancer.

In a still more preferred embodiment, an antibody, or fragments thereof, that binds to a peptide comprising the sequence SVSDV are incorporated into anti-cancer therapeutics or diagnostics for the diagnosis, treatment or prevention of a MUC1* positive cancer.

In a yet still more preferred embodiment, an antibody, or fragments thereof, that binds to a peptide comprising some or all of the sequence PFPFSAQSGA are incorporated into anti-cancer therapeutics or diagnostics for the diagnosis, treatment or prevention of a MUC1* positive cancer. As an anti-cancer treatment, the selected antibodies or fragments thereof are incorporated into a CAR, BiTE, ADC or bi-specific and then administered to a patient diagnosed with or at risk of developing a MUC1* positive cancer.

### Consensus Sequences

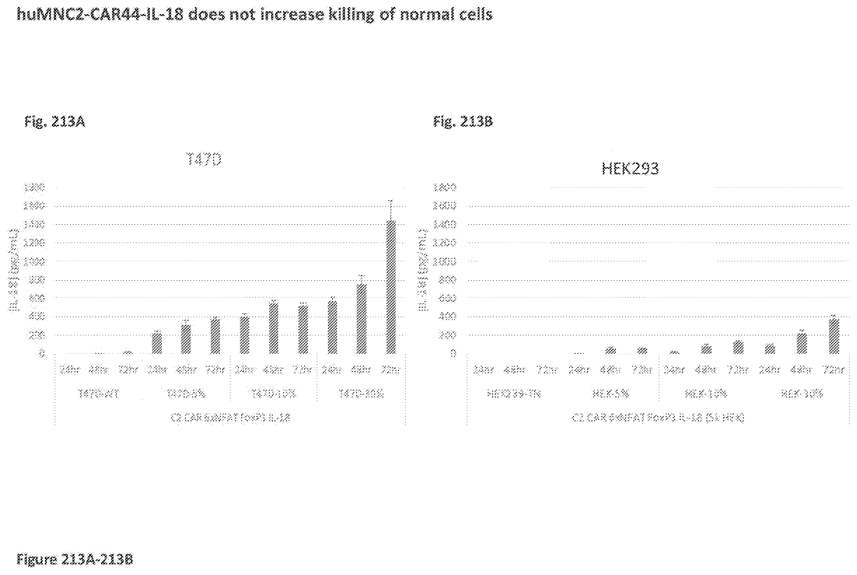

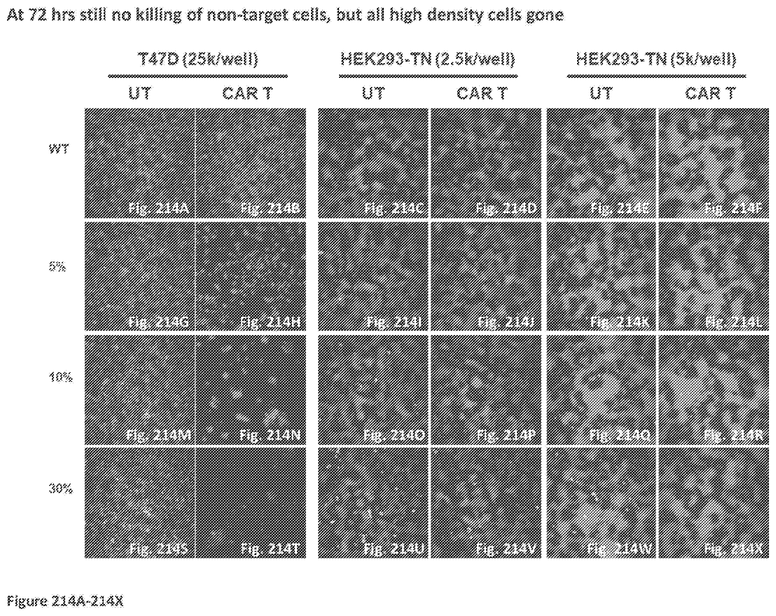

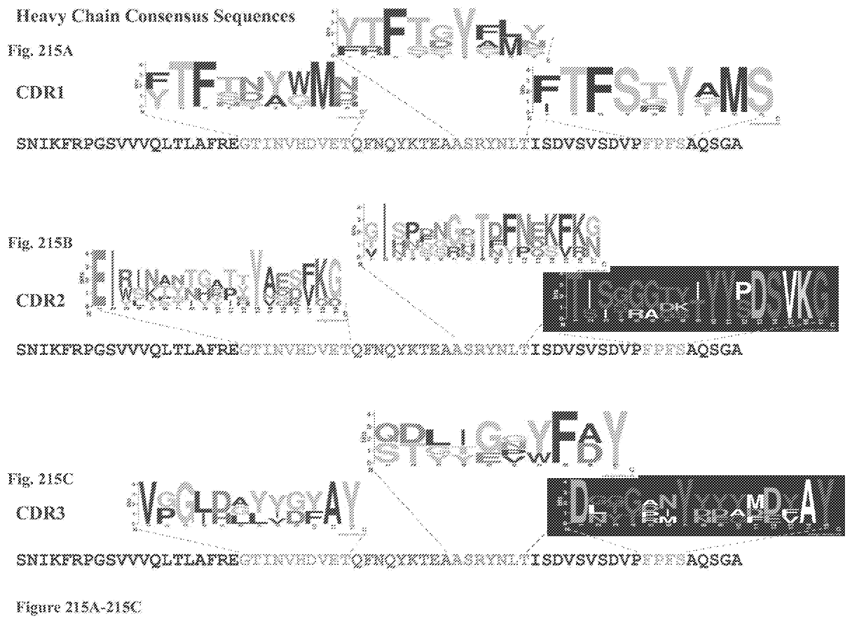

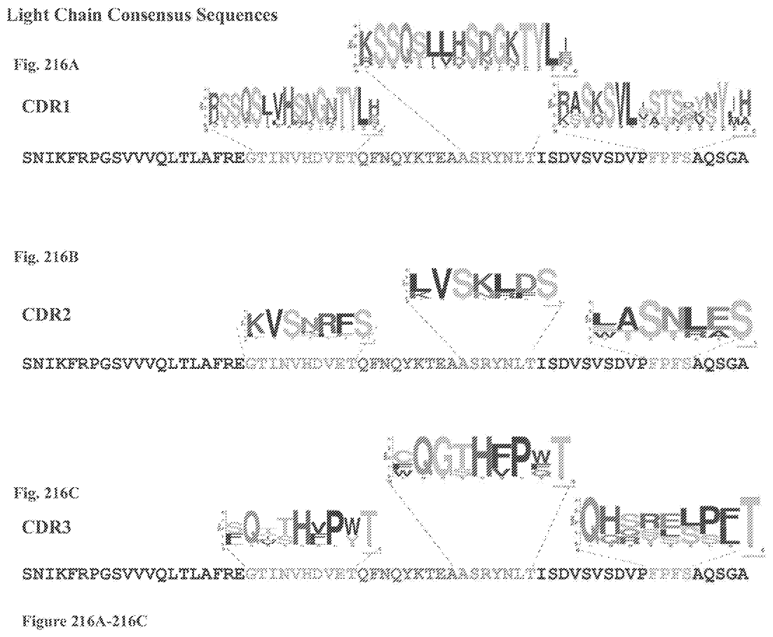

Antibodies of the invention were categorized according to cognate epitope. Sequences of their respective heavy chain CDRs are shown in Table 5. Sequences of their respective light chain CDRs are shown in Table 6. Consensus sequences for CDR1, CDR2 and CDR3 for each epitope-specific set of antibodies were computer generated. FIG. 215 and FIG. 216 show how the CDR consensus sequences change as the position of the antibodies' cognate epitope moves from the membrane-proximal portion of PSMGFR toward the more distal portions.

As can be seen in Table 5 and Table 6, the sequences for CDR1 and CDR2 for antibodies that bind to epitopes within the 10 membrane-proximal (C-terminal) portion of PSMGFR peptide closely adhere to the consensus sequence.

TABLE 5
HEAVY CHAIN CDRs
GTINVHDVETQFNQYKTEAASRYNLTISDVSVSDVPFPFSAQSGA

| Epitope | Name | CDR1 | CDR2 | CDR3 |
|---|---|---|---|---|
| FPES | MNC2 | FTFSGYAMS | TISSGGTYIYYPDSVKG | -LGGDNYYEYEDV-- |
| FPFS | MNE6 | FTFSRYGMS | TISGGGTYIYYPDSVKG | DNYGRNYDYGMDY-- |
| FPES | 20A10 | FTFSTYAMS | -SIGRAGSTYYSDSVKG | ---GPIYNDYDEFAY |
| FPFS | 3C2B1 | ITFSTYTMS | TISTGGDKTYYSDSVKG | -GTTAMYYYAMDY- |
| Consensus Sequence | | [symbol] | [symbol] | [symbol] |
| SVSDV | 5C6F3 | FTESTYAMS | AISNGGGYTYYPDSLKG | RYYDHYFDY |
| ASRYNLT | 25E6 | FTFSSYGMS | TISNGGRHTFYPDSVKG | QTGTEGWFAY |
| ASRYNLT | MNC3 | YRFTDYAMN | VISTESGNTNENQKFKG | SDYYGPYFDY |
| ASRYNLT | 18G12 | YTFTGYFLY | GINPDNGGIDENEKERN | --LIGNY--- |
| ASRYNLT | 28F9 | YTFTGYFLY | GIHPSNGDTDENEKFKN | --LIGVY--- |
| Consensus Sequence | | [symbol] | [symbol] | [symbol] |
| QFNQYKTEA | 1E4 | YAFSTYWMN | QIYPGDSDTNYNGKEKG | GNHASMDY |
| GTINVHDVET | 18B4 | FTENDAWMD | EIRSTANIHTTYYAESVQ | -----LLYGFAY |
| | | | G | |
| GTINVHDVET | 29H1 | FTFSDAWMD | EIRSKATNHATYYAESVK | -----LLYGFAY |
| | | | G | |
| GTINVHDVET | 31A1 | YTFTSYWMH | YINPSTGYTEYNQKFKD | -----AYIDY-- |
| GTINVHDVET | 32C1 | FTFSNYWMN | EIRLKSNNYAIHYAESVK | VPGLDAY----- |
| | | | G | |
| GTINVHDVET | 39H5 | YTFTNYGMN | WINTYTGEPTYVGDEKG | --GIHGYVDY-- |
| GTINVHDVET | 3C5 | YTFTNYGMN | WINTYTGKPTYADDEKG | -GGLDGYYGY- |
| Consensus Sequence | | [symbol] | [symbol] | [symbol] |

TABLE 6
LIGHT CHAIN CDRs
GTINVHDVETQFNQYKTEAASRYNLTISDVSVSDVPFPFSAQSGA

| Epitope | Name | CDR1 | CDR2 | CDR3 |
|---|---|---|---|---|
| FPFS | MNC2 | RASKS--VSTSGYSYMH | LASNLES | QHSRELPFT |
| FPFS | MNE6 | -------SATSSVSYIH | STSNLAS | QQRSSSPFT |
| FPFS | 20A10 | KSSQSVLYSSNQKNYLA | WASTRES | -HQYLSSLT |
| FPFS | 3C2B1 | RASKS---ISTSDYNYIH | LASNLES | QHSRELPLT |
| Consensus Sequence | | [symbol] | [symbol] | [symbol] |
| SVSDV | 5C6F3 | RSSQTIVHSNGNTYLE | KVSNRFS | FQDSHVPLT |
| ASRYNLT | 25E6 | KSSQSLLDSDGKTYLN | LVSKLDS | WQGTHEPQT |
| ASRYNLT | MNC3 | RSSQTIVHSNGNTYLE | KVSNRFS | FQGSHVPFT |
| ASRYNLT | 18G12 | KSSQSLLHSDGKTYLI | LVSKLDS | CQGTHEPWT |
| ASRYNLT | 28F9 | KSSQSLLHSDGKTYLI | LVSKLDS | CQGTHEPWT |
| Consensus Sequence | | [symbol] | [symbol] | [symbol] |
| QFNQYKTEA | 1E4 | RSSQSLVHSNGNTYLH | KVSNRFS | SQKTHVPWT |
| GTINVHDVET | 18B4 | RTSQSLVHSNGNTYLH | KVSSRES | SQNTHVPYT |
| GTINVHDVET | 29H1 | RSGQSLVHSNGHTYLH | KVSNRFS | SQTTHVPWT |
| GTINVHDVET | 31A1 | RSSQSIVHSNGNTYLE | KVSNRFS | FQVSHFPWT |
| GTINVHDVET | 32C1 | RSSQSLVHSNGNTYLH | KVSNRFS | SQITHVPYT |
| GTINVHDVET | 39H5 | RSSQSIVHRNGNTYL- | KVSNRFS | FQGSHLPWT |
| GTINVHDVET | 3C5 | KSSQSLLHSKGKTYLN | LVSKLES | LQTTHEPWT |
| Consensus Sequence | | [symbol] | [symbol] | [symbol] |

Whereas Heavy Chain CDR1 for MNC2 is FTFSGYAMVS, with the amino acids numbered from left to right 1 through 9, the consensus of other antibodies that bind to that portion of PSMGFR is: F or I at position 1, T at position 2, F at position 3, S at position 4, T, G, or R at position 5, Y at position 6, A, G or T at position 7, M at position 8 and S at position 9.

In a preferred embodiment an antibody is chosen for the treatment, prevention or diagnosis of cancer based on having a heavy chain CDR1 that is at least 90% identical to a CDR1 comprising the following amino acids at the specified positions: F or I at position 1, T at position 2, F at position 3, S at position 4, T, G, or R at position 5, Y at position 6, A, G or T at position 7, M at position 8 and S at position 9.

Whereas Heavy Chain CDR2 for MNC2 is TISSGGTYIYYPDSVKG, with the amino acids numbered from left to right 1 through 17, the consensus of other antibodies that bind to that portion of PSMGFR is: T at position 1, I or S at position 2, I or S at position 3, G or R at position 5, G or A at position 6, T or I at position 9, Y at position 10, Y at position 11, P or S at position12 and DSVKG for positions 13-17.

In a preferred embodiment an antibody is chosen for the treatment, prevention or diagnosis of cancer based on having a heavy chain CDR2 that is at least 90% identical to a CDR2 comprising the following amino acids at the specified positions: T at position 1, I or S at position 2, I or S at position 3, G or R at position 5, G or A at position 6, T or I at position 9, Y at position 10, Y at position 11, P or S at position12 and DSVKG for positions 13-17.

Whereas Heavy Chain CDR3 for MNC2 is -LGGDNYYEYFDV--, with the amino acids numbered from left to right 1 through 15, the consensus of other antibodies that bind to that portion of PSMGFR is: G, L, or N at position 2, G or T at position 4, Y at position 7, D or E at position 12, A at position 14, and Y at position 15.

In a preferred embodiment an antibody is chosen for the treatment, prevention or diagnosis of cancer based on having a heavy chain CDR3 that is at least 90% identical to a CDR3 comprising the following amino acids at the specified positions: G, L, or N at position 2, G or T at position 4, Y at position 7, D or E at position 12, A at position 14, and Y at position 15.

Whereas Light Chain CDR1 for MNC2 is RASKS--VSTSGYSYMH, with the amino acids numbered from left to right 1 through 17, the consensus of other antibodies that bind to that portion of PSMGFR is: K or R at position 1, A or S at position 2, S at position 3, K or Q at position 4, S at position 5, V at position 6, L at position 7, T or S at position 10, Y at position 15, and I, L or M at position 16.

In a preferred embodiment an antibody is chosen for the treatment, prevention or diagnosis of cancer based on having a light chain CDR1 that is at least 90% identical to a CDR1 comprising the following amino acids at the specified positions: K or R at position 1, A or S at position 2, S at position 3, K or Q at position 4, S at position 5, L or V at position 6, L at position 7, T or S at position 10, Y at position 15, and I, L or M at position 16.

Whereas Light Chain CDR2 for MNC2 is LASNLES, with the amino acids numbered from left to right 1 through 7, the consensus of other antibodies that bind to that portion of PSMGFR is: L or W, or S at position 1, A or T at position 2, S at position 3, N or T at position 4, L or R at position 5, E or A at position 6, and S at position 7.

In a preferred embodiment an antibody is chosen for the treatment, prevention or diagnosis of cancer based on having a light chain CDR2 that is at least 90% identical to a CDR2 comprising the following amino acids at the specified positions: L or W, or S at position 1, A or T at position 2, S at position 3, N or T at position 4, L or R at position 5, E or A at position 6, and S at position 7.

Whereas Light Chain CDR3 for MNC2 is QHSRELPFT, with the amino acids numbered from left to right 1 through 9, the consensus of other antibodies that bind to that portion of PSMGFR is: Q at position 1, H or Q at position 2, S, Q or R at position 3, R, S or Y at position 4, E, L, or S at position 5, L or S at position 6, P or S at position 7, F or L at position 8 and T at position 9.

In a preferred embodiment an antibody is chosen for the treatment, prevention or diagnosis of cancer based on having a light chain CDR3 that is at least 90% identical to a CDR3 comprising the following amino acids at the specified positions: Q at position 1, H or Q at position 2, S, Q or R at position 3, R, S or Y at position 4, E, L, or S at position 5, L or S at position 6, P or S at position 7, F or L at position 8 and T at position 9.

Another set of antibodies was generated and resultant clones were tested for their ability to bind to PSMGFR, N-10 and C-10 peptides. Antibody clones that bound to PSMGFR and N-10 peptides, but not to the C-10 peptide were selected. These antibodies were sequenced. Table 7 shows the sequences of the heavy chain CDRs for cancer-specific antibodies MNC2, MNE6, 20A10, 3C2B1, plus new antibodies B2, B7, 8C7F3, H11 and B9. Table 8 shows the sequences of the light chain CDRs for cancer-specific antibodies MNC2, MNE6, 20A10, 3C2B1, plus new antibodies B2, B7, 8C7F3, H11 and B9. Consensus sequences for the heavy and light chain CRDs were generated and are shown in Table 7 and Table 8. Although antibodies 5C6F3 and 25E6 showed great cancer specificity in IHC tissue studies and they both bound to the PSMGFR and N-10 peptides, but not to the C-10 peptide, epitope mapping showed that they bound to epitopes that were a bit N-terminal to the epitopes to which MNC2, MNE6, 20A10 and 3C2B1 bound. For this reason, consensus sequences were generated for MNC2, MNE6, 20A10, 3C2B1 and the new antibodies plus consensus sequences were generated for all the antibodies that bound to N-10 but not to C-10.

As can be seen in Table 7 and Table 8, the sequences for CDR1, CDR2 and CDR3 for antibodies that require for binding the 10 membrane-proximal (C-terminal) amino acids of PSMGFR peptide closely adhere to a common consensus sequence.

TABLE 7
HEAVY CHAIN CDRs for antibodies that share broader epitope in
that they cannot bind to the C-10 peptide
GTINVHDVETQFNQYKTEAASRYNLTISDVSVSDVPFPFSAQSGA

| Epitope | Name | CDR1 | CDR2 | CDR3 |
|---|---|---|---|---|
| FPFS | MNC2 | FTFSGYAMS | TISSGGTYIYYPDS | -LGGDNYYEYFDV-- |
| | | | VKG | |
| FPFS | MNE6 | FTFSRYGMS | TISGGGTYIYYPDS | DNYGRNYDYGMDY-- |
| | | | VKG | |
| FPFS | 20A10 | FTFSTYAMS | SIGRAGSTYYSDSV | ---GPIYNDYDEFAY |
| | | | KG | |
| FPES | 3C2B1 | ITFSTYTMS | TISTGGDKTYYSDS | -GTTAMYYYAMDY-- |
| | | | VKG | |
| PFPFSAQS | B2 | FAFSTEAMS | AISNGGGYTYYPDT | ----RYYDLYFDL-- |
| GA | | | LKG | |
| PFPFSAQS | B7 | FTFSRYGMS | TISSGGTYIYYPDS | DNYGSSYDYAMDY-- |
| GA | | | VKG | |
| PFPFSAQS | 8C7F3 | FTFSTYAMS | AISNGGGYTYYPDS | ---RYYDHYFDY-- |
| GA | | | LKG | |
| PFPFSAQS | H11 | FAFSTEAMS | AISNGGGYTYYPDT | ---RYYDLYFDL-- |
| GA | | | LKG | |
| PFPFSAQS | B9 | FTFSRYGMS | TISSGGTYIYYPDS | DNYGSSYDYAMDY-- |
| GA | | | VKG | |
| Consensus Sequence | | [symbol] | [symbol] | [symbol] |
| -all of | | | | |
| antibodies | | | | |
| above | | | | |
| SVSDV | 5C6F3 | FTFSTYAMS | AISNGGGYTYYPDS | RYYDHYFDY |
| | | | LKG | |
| ASRYNLT | 25E6 | FTFSSYGMS | TISNGGRHTFYPDS | QTGTEGWFAY |
| | | | VKG | |
| Consensus Sequence | | [symbol] | [symbol] | [symbol] |
| -all of | | | | |
| antibodies | | | | |
| above | | | | |

TABLE 8
LIGHT CHAIN CDRs for antibodies that
share broader epitope in that they
cannot bind to the C-10 peptide
GTINVHDVETQFNQYKTEAASRYNLTISDVSVSDVPFPFSAQSGA

| Epitope | Name | CDR1 | CDR2 | CDR3 |
|---|---|---|---|---|
| FPFS | MNC2 | RASKS--VSTSGYSYMH | LASNLES | QHSRELPFT |
| FPFS | MNE6 | -------SATSSVSYIH | STSNLAS | QQRSSSPFT |
| FPFS | 20A10 | KSSQSVLYSSNQKNYLA | WASTRES | -HQYLSSLT |
| FPES | 3C2B1 | RASKS---ISTSDYNYIH | LASNLES | QHSRELPLT |
| PFPFSAQSGA | B2 | RSSQNIV-HSNGNTYLE | KVSNRFS | FQDSHVPLT |
| PFPFSAQSGA | B7 | RSSQTIV-HSNGNTYLE | KVSNRFS | FQDSHVPLT |
| PFPFSAQSGA | 8C7F3 | --RASESVATYGNNEMQ | LASTLDS | QQNNEDPPT |
| PFPFSAQSGA | H11 | RSSQNIV-HSNGNTYLE | KVSNRFS | FQDSHVPLT |
| PFPFSAQSGA | B9 | -------SASSSVSYMH | TTSNLAS | QQRSSYPF- |
| Consensus Sequence- | | [symbol] | [symbol] | [symbol] |
| all of | | | | |
| antibodies | | | | |
| above | | | | |
| SVSDV | 5C6F3 | RSSQTIVHSNGNTYLE | KVSNRFS | FQDSHVPLT |
| ASRYNLT | 25E6 | KSSQSLLDSDGKTYLN | LVSKLDS | WQGTHFPQT |
| Consensus Sequence- | | [symbol] | [symbol] | [symbol] |
| all | | | | |
| antibodies | | | | |

Whereas Heavy Chain CDR1 for MNC2 is FTFSGYAMS, with the amino acids numbered from left to right 1 through 9, the consensus sequence of MNC2, MNE6, 20A10, 3C2B1 and new antibodies B2, B7, 8C7F3, H11 and B9 is: F or I at position 1, T or A at position 2, F at position 3, S at position 4, T, G, or R at position 5, Y or F at position 6, A, G or T at position 7, M at position 8 and S at position 9. The underlined amino acids at positions 2 and 6 are the only additional variants to the consensus sequence generated for cancer-specific antibodies MNC2, MNE6, 20A10, 3C2B1 alone.

As can be seen in Table 7, the inclusion of antibodies 5C6F3 and 25E6 into the generation of consensus sequence did not change in any way the consensus sequence for heavy chain CDR1 that describes a cancer-specific anti-MUC1* antibody.

In a preferred embodiment an antibody is chosen for the treatment, prevention or diagnosis of cancer based on having a heavy chain CDR1 that is at least 90% identical to a CDR1 comprising the following amino acids at the specified positions: F or I at position 1, T or A at position 2, F at position 3, S at position 4, T, G, or R at position 5, Y or F at position 6, A, G or T at position 7, M at position 8 and S at position 9.

Whereas Heavy Chain CDR2 for MNC2 is TISSGGTYIYYPDSVKG, with the amino acids numbered from left to right 1 through 17, the consensus sequence of MNC2, MNE6, 20A10, 3C2B1 and new antibodies B2, B7, 8C7F3, H11 and B9 is:
T or A at position 1, I or S at position 2, I or S at position 3, N, S, T or G at position 4, G or R at position 5, G or A at position 6, G, T, or D at position 7, Y, K or S at position 8, T or I at position 9, Y at position 10, Y at position 11, P or S at position12 and D at position 13, S or T at position 14, V or L at position 15 and KG for positions 16-17. The underlined amino acids indicate how this more inclusive consensus sequence differs from the consensus sequence generated for MNC2, MNE6, 20A10 and 3C2B1 alone. Of the 17 amino acids in heavy chain CDR2, the consensus sequence for all nine antibodies differs from the consensus sequence for the original cancer-specific four by only 4 amino acids. Note that 2 of the 4 variants are homologous changes, T for S and L for V, which generally do not significantly impact the structure or specificity of a protein.

As can be seen in Table 7, the inclusion of antibodies 5C6F3 and 25E6 into the generation of consensus sequence for heavy chain CDR2 only changed the consensus sequence by the addition of two other possible amino acids: a possible H at position 8, and a possible F at position 10, for a heavy chain CDR2 that describes a cancer-specific anti-MUC1* antibody. We note that the change of Y to F at position 10 is a homologous change, which generally does not significantly impact the structure or specificity of a protein.

In a preferred embodiment an antibody is chosen for the treatment, prevention or diagnosis of cancer based on having a heavy chain CDR2 that is at least 90% identical to a CDR2 comprising the following amino acids at the specified positions: T or A at position 1, I or S at position 2, I or S at position 3, N, S, T or G at position 4, G or R at position 5, G or A at position 6, G, T, or D at position 7, Y, K, H or S at position 8, T or I at position 9, Y or F at position 10, Y at position 11, P or S at position12 and D at position 13, S or T at position 14, V or L at position 15 and KG for positions 16-17.

Whereas Heavy Chain CDR3 for MNC2 is LGGDNYYEYFDV, with the amino acids numbered from left to right 2 through 13, the consensus sequence of MNC2, MNE6, 20A10, 3C2B1 and new antibodies B2, B7, 8C7F3, H11 and B9 is:
G, L, or N at position 2, G, T, or Y at position 3, G or T at position 4, A, D, P, R, or S at position 5, Y, M, I or S at position 6, Y at position 7, D, Y, or N at position 8, E, D, Y, L or H at position 9, Y, A, or G at position 10, M, D or F at position 11, D or E at position 12, V, F, Y or L at position 13, and AY at position 14-15. The underlined amino acids indicate how this more inclusive consensus sequence differs from the consensus sequence generated for MNC2, MNE6, 20A10 and 3C2B1 alone. Of the 15 amino acids in heavy chain CDR3, the consensus sequence for all nine antibodies differs from the consensus sequence for the original cancer-specific four by 7 amino acids, with 3 of the 7 substitutions at position 6. For this reason, we conclude that the amino acid at position 6 can be varied without altering the specificity of the antibody.

Analysis of the consensus sequence generated with the inclusion of antibodies 5C6F3 and 25E6 highlighted which amino acids were conserved among all eleven antibodies. For this reason, our preferred consensus sequence for heavy chain CDR3 defines amino acids at positions 2, 3, 4, 7, 10, 11, 12, 14 and 15, where for 11 antibodies, there were 3 or less variants at these positions.

In a preferred embodiment an antibody is chosen for the treatment, prevention or diagnosis of cancer based on having a heavy chain CDR3 that is at least 90% identical to a CDR3 comprising the following amino acids at the specified positions: G, L, or N at position 2, G, T, or Y at position 3, G or T at position 4, Y at position 7, Y, A, or G at position 10, M, D or F at position 11, D or E at position 12 and AY at position 14-15.

Whereas Light Chain CDR1 for MNC2 is RASKS--VSTSGYSYMH, with the amino acids numbered from left to right 1 through 17, the consensus sequence of MNC2, MNE6, 20A10, 3C2B1 and new antibodies B2, B7, 8C7F3, H11 and B9 is:
K or R at position 1, A or S at position 2, S or R at position 3, K, Q or A at position 4, S, N or T at position 5, V, I, E, or K at position 6, L, V or S at position 7, S, Y, I or V at position 8, A, S, or H at position 9, T or S at position 10, N, S, or Y at position 11, G, S, D, or Q at position 12, V, Y, K or N at position 13, N, S, or T at position 14, Y or F at position 15, and I, L or M at position 16, and H, A, E or Q at position 17. The underlined amino acids indicate how this more inclusive consensus sequence differs from the consensus sequence generated for MNC2, MNE6, 20A10 and 3C2B1 alone. Of the 17 amino acids in light chain CDR1, the consensus sequence for all nine antibodies differs from the consensus sequence for the original cancer-specific four by 13 amino acids. 4 of the 13 are homologous substitutions, which in general do not significantly alter the structure or specificity of the protein. Of the remaining 9 substitutions, 1 is at position 4, 1 is at position 5, 3 are at position 6, 1 is at position 7, 1 is at position 11, and 2 are at is at position 17. The inclusion of the 5 new antibodies did not alter the amino acids, excluding homologous substitutions, at positions 1, 2, 3, 8, 9, 10, 12, 13, 14, 15 or 16. For this reason, we conclude that the conserved consensus sequence for light chain CDR1 that defines a MUC1* cancer-specific antibody comprises the amino acids given above for positions 1, 2, 3, 8, 10, 12, 13, 14, 15 and 16.

Analysis of the consensus sequence generated with all the antibodies, including 5C6F3 and 25E6 further altered the consensus sequence for light chain CDR1 with amino acid substitutions as follows: L at position 6; D at position 9; D at position 11 and N at position 17. We note that none of these substitutions were at positions that were invariant for the original four cancer-specific antibodies plus the five new antibodies. Thus, we conclude that a conserved consensus sequence for light chain CDR1 that defines at least 90% identity of a cancer-specific antibody comprises amino acids defined above at positions 1, 2, 3, 8, 10, 12, 13, 14, 15 and 16.

In a preferred embodiment an antibody is chosen for the treatment, prevention or diagnosis of cancer based on having a light chain CDR1 that is at least 90% identical to a CDR1 comprising K or R at position 1, A or S at position 2, S or R at position 3, S, Y, I or V at position 8, T or S at position 10, G, S, D, or Q at position 12, V, Y, K or N at position 13, N, S, or T at position 14, Y or F at position 15, and I, L or M at position 16.

Whereas Light Chain CDR2 for MNC2 is LASNLES, with the amino acids numbered from left to right 1 through 7, the consensus sequence of MNC2, MNE6, 20A10, 3C2B1 and new antibodies B2, B7, 8C7F3, H11 and B9 is: L, W, S, T or K at position 1, A, T or V at position 2, S at position 3, N or T at position 4, L or R at position 5, E, A, F or D at position 6, and S at position 7. The underlined amino acids indicate how this more inclusive consensus sequence differs from the consensus sequence generated for MNC2, MNE6, 20A10 and 3C2B1 alone.

In a preferred embodiment an antibody is chosen for the treatment, prevention or diagnosis of cancer based on having a light chain CDR2 that is at least 90% identical to a CDR2 comprising the following amino acids at the specified positions: L, W, S, T or K at position 1, A, T or V at position 2, S at position 3, N or T at position 4, L or R at position 5, E, A, F or D at position 6, and S at position 7. Of the 7 positions, the inclusion of the five new antibodies introduced 5 substitutions of which only 2 were not homologous substitutions.

Analysis of the consensus sequence generated with all the antibodies, including 5C6F3 and 25E6 further altered the consensus sequence for light chain CDR2 with amino acid substitutions as follows: K at position 4, which is a substitution that is homologous to N.

In a preferred embodiment an antibody is chosen for the treatment, prevention or diagnosis of cancer based on having a light chain CDR2 that is at least 90% identical to a CDR2 comprising: A, T or V at position 2, S at position 3, N, T, or K at position 4, L or R at position 5, E, A, F or D at position 6, and S at position 7.

Whereas Light Chain CDR3 for MNC2 is QHSRELPFT, with the amino acids numbered from left to right 1 through 9, t the consensus sequence of MNC2, MNE6, 20A10, 3C2B1 and new antibodies B2, B7, 8C7F3, H11 and B9 is: Q or F at position 1, H or Q at position 2, S, Q, R, D or N at position 3, R, S, Y or N at position 4, E, L, S or H at position 5, L, S, V, D or Y at position 6, P or S at position 7, F, L or P at position 8 and T at position 9. The underlined amino acids indicate how this more inclusive consensus sequence differs from the consensus sequence generated for MNC2, MNE6, 20A10 and 3C2B1 alone.

Analysis of the consensus sequence generated with all the antibodies, including 5C6F3 and 25E6 further altered the consensus sequence for light chain CDR2 with amino acid substitutions as follows: W at position 1; G at position 3; T at position 4; F at position 5; Q at position 8.

In a preferred embodiment an antibody is chosen for the treatment, prevention or diagnosis of cancer based on having a light chain CDR3 that is at least 90% identical to a CDR2 comprising: Q, F or W at position 1, H or Q at position 2, R, S, T, Y or N at position 4, E, L, S or H at position 5, L, S, V, D or Y at position 6, P or S at position 7, and T at position 9.

Other General Strategy for Using Antibodies, Antibody Fragments and CARs that Target the Extracellular Domain of MUC1*

In another aspect, the invention is directed to a composition that includes at least two different plasmids transfected into the same immune cell, wherein the first encodes a CAR comprising an antibody fragment, scFv, or peptide that binds to a tumor antigen and the other encodes a gene that is not a CAR, wherein the gene that is not a CAR is expressed from an inducible promoter that is activated by elements of an activated immune cell. In one aspect, the immune cell is a T cell or an NK cell. In one aspect the immune cell is derived from a stem cell that has been directed to differentiate to that immune cell type in vitro. In another aspect, a CAR containing sequences of the antibody are expressed in a stem cell, which then may be differentiated into an immune cell. In one aspect the CAR comprises an antibody fragment, scFv or peptide that binds to the extra cellular domain of MUC1*. In one aspect the CAR comprises an scFv derived from MNC2, MNE6, 20A10, 3C2B1, 5C6F3, 25E6, 18G12, 28F9, 1E4, B12, B2, B7, B9, 8C7F3, or H11. In one aspect the non-CAR species is a cleavage enzyme. In one aspect the cleavage enzyme is MMP2, MMP3, MMP9, MMP13, MMP14, MMP16, ADAM10, ADAM17, ADAM28 or catalytically active fragments thereof. In another aspect the non-CAR species is a cytokine. In one aspect, the Cytokine is IL-7. In one aspect the cytokine is IL-15. In one aspect the cytokine is IL-12. In one aspect the cytokine is IL-18. The sequence of an activated IL-18 is given (SEQ ID NOS:1637-1638). Two examples of NFAT-inducible IL-18 embedded in the Foxp3 enhancer region are given (SEQ ID NOS:1639-1640). Two examples of NFAT-inducible IL-18 embedded in the IL-2 enhancer region are given (SEQ ID NOS:1641-1642). In one case, there are three (3) NFAT response elements and in the other acse there are six (6) NFAT response elements. The number of NFAT response elements can be varied in order to get the desired amount of IL-18 expressed upon CAR T cell recognition of the target. Examples of antibodies of the invention incorporated into CARS with inducible IL-18 are shown as: murine or human MNC2 in a CAR with a 4-1BB or CD28 co-stimulatory domain plus inducible IL-18 (SEQ ID NOS:1643-1646), or also with a 1XX mutated CD3-zeta (SEQ ID NOS:1647-1650); murine or human MNE6 in a CAR with a 4-1BB or CD28 co-stimulatory domain plus inducible IL-18 (SEQ ID NOS:1651-1654), or also with a 1XX mutated CD3-zeta (SEQ ID NOS:1655-1658); murine or human 20A10 in a CAR with a 4-1BB or CD28 co-stimulatory domain plus inducible IL-18 (SEQ ID NOS:1659-1662), or also with a 1XX mutated CD3-zeta (SEQ ID NOS:1663-1666); murine or human 25E6 in a CAR with a 4-1BB or CD28 co-stimulatory domain plus inducible IL-18 (SEQ ID NOS:1667-1670), or also with a 1XX mutated CD3-zeta (SEQ ID NOS:1671-1674). In another aspect the cytokine is IL-7 and IL-15. In one case expression of the non-CAR species is induced by elements of an activated immune cell. In one aspect the element of an activated immune cell is an NFAT. In one aspect the NFAT is NFATc1, NFATc3 or NFATc2. Cytokines IL-7, IL-15, IL-12 and IL-18 are known to promote T cell persistence. In one aspect of the invention an immune cell described above is administered to a patient for the treatment or prevention of cancer. In one aspect of the invention, the cancer is a MUC1 positive cancer or a MUC1* positive cancer.

In addition to making CAR T cells that also induce expression of a cleavage enzyme, we made CAR T cells that also induce local and transient expression of IL-18. Many of the T cell based inducible systems reported insert the gene to be inducibly expressed into an IL-2 promoter or enhancer. We compared inducible expression off an IL-2 promoter/enhancer to inducible expression off of a portion of the Foxp3 enhancer. In this particular example, human T cells were transduced with both huMNC2-CAR44 and an NFAT inducible IL-18, wherein the I1-18 gene was either inserted into an IL-2 promoter or the Foxp3 enhancer region. It is known in the field that a major problem with CAR Ts with inducible second factors is that the second factor is leaky, meaning that significant expression of the second factor occurs without activation of the CAR T cell. The other problem with existing inducible systems is the length of time that goes by between when the CAR T cell is activated and the second factor is induced is typically very long so that the cell secreting the second factor may be far away from the tumor by the time the second factor is expressed.

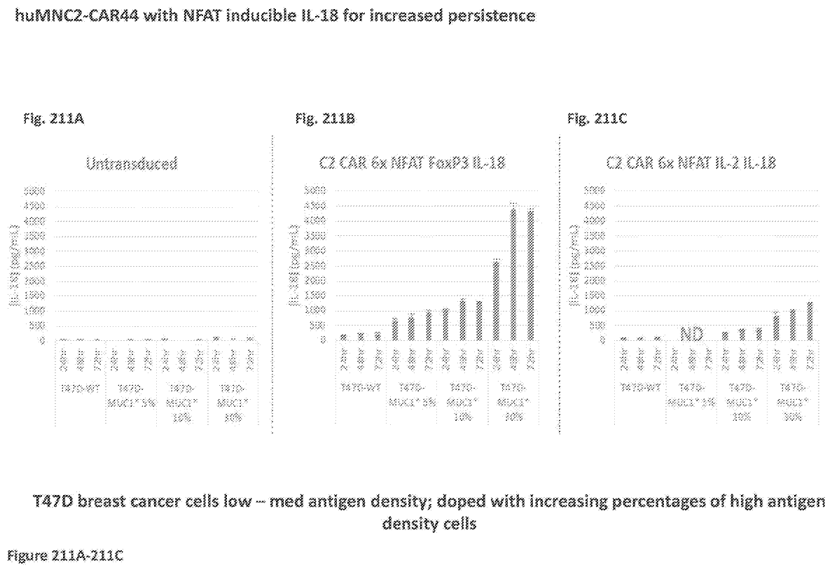

FIG. 211A-211C show graphs of an ELISA experiment measuring the amount of IL-18 secreted into the condition media of huMNC2-CAR44 T cells, which also bear an NFAT inducible IL-18, co-cultured with MUC1* positive cancer cells. As a method of inducing varying levels of IL-18 expression, we co-cultured the CAR T cells with cancer cells doped with increasing amounts of cells that were engineered to express even more MUC1*. In these figures we show T47D cancer cells that are either wild-type, or doped with 5%, 10% or 30% of the T47D cells expressing more MUC1*. FIG. 211A shows the graph of IL-18 secreted into the supernatant of T47D breast cancer cells co-cultured with untransduced human T cells. FIG. 211B shows the graph of IL-18 secreted into the supernatant of T47D breast cancer cells co-cultured with huMNC2-CAR44 T cells that also bore an NFAT inducible IL-18 gene inserted into a portion of the Foxp3 enhancer. FIG. 211C shows the graph of IL-18 secreted into the supernatant of T47D breast cancer cells co-cultured with huMNC2-CAR44 T cells that also bore an NFAT inducible IL-18 gene inserted into a portion of the IL-2 enhancer. As can be seen in the figure, the Foxp3 system induces rapid and robust expression of IL-18, which is significantly faster and higher than that of the same construct in an IL-2 promoter. In this example, the IL-18 gene is inserted downstream of six (6) NFAT response elements, however one can attenuate the amount of the second factor by using a lesser number of response elements or enhance the amount by increasing the number of NFAT response elements.

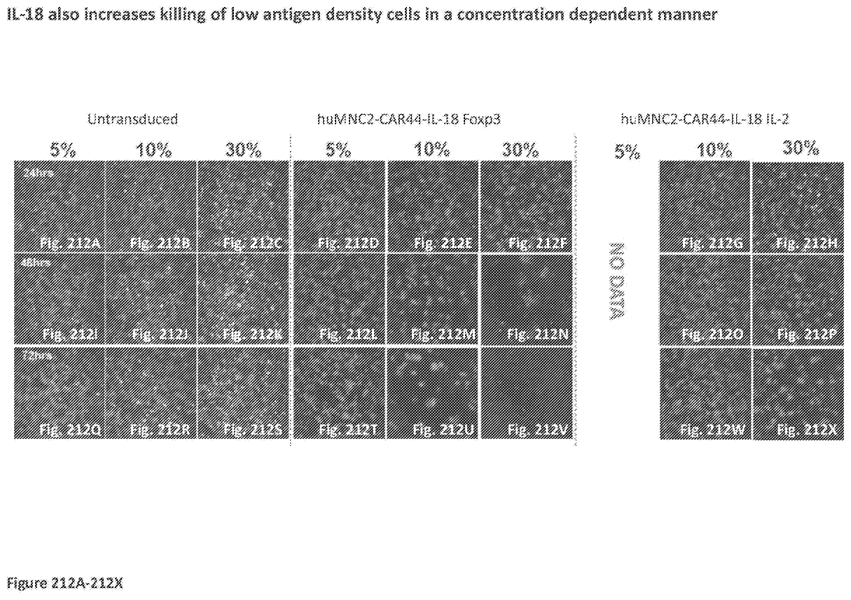

It has been reported that IL-18 increases persistence of CAR T cells in vivo. However, we observed an unexpected result. In a dose-dependent manner, secretion of IL-18 increased the killing of low antigen density cells by the CAR T cells. We differentially labeled the T47D-wt cells (red: mCherry) and those T47Ds that were transduced to express more MUC1* (green: GFP). FIG. 212A-212X shows photographs of T47D breast cancer cells (red) doped with varying percentages of T47D cells engineered to express more MUC1* (green). The target cancer cells have been co-cultured with huMNC2-CAR44 T cells with NFAT inducible IL-18 wherein the IL-18 gene has been inserted into either the Foxp3 enhancer/promoter or the IL-2 enhancer/promoter. FIGS. 212A-212C, 212I-212K, and 212Q-212S show the cancer cells co-cultured with untransduced T cells. FIGS. 212D-212F, 212L-212N, and 212T-212V show the cancer cells co-cultured with hiMNC2-CAR44 T cells with the NFAT inducible IL-18 gene inserted into the Foxp3 enhancer/promoter. FIGS. 212G-212H, 2120-212P, and 212W-212X show the cancer cells co-cultured with hiMNC2-CAR44 T cells with the NFAT inducible IL-18 gene inserted into the IL-2 enhancer/promoter. As can be seen in the figure, the low antigen density T47D-wt type cells (red) are being killed when doped with higher percentages of cells that express more MUC1* and thus secrete more IL-18. The experiment shows that this is not just a bystander effect, because the cells expressing IL-18 off of the IL-2 promoter, which expresses much lower levels of IL-18, do not kill the low antigen density cells even when they are doped with 30% cells expressing more MUC1*.

We then showed that the CAR T mediated killing is specific for the CAR T specific antigen. We performed a similar experiment, wherein control, MUC1/MUC1* negative cells were doped with 5%, 10% or 30% of the T47D cells expressing more MUC1*, and co-cultured with MUC1* specific CAR T cells. FIG. 213A-213B shows graphs of ELISA experiments in which levels of IL-18 secreted into the conditioned media are measured for huMNC1-CAR44 T cells with NFAT inducible IL-18 gene, inserted into the Foxp3 enhancer or promoter, co-cultured with either MUC1* positive cancer cells or MUC1 negative non-cancerous cells. FIG. 213A shows IL-18 secretion from huMNC2-CAR44 T cells with NFAT inducible IL-18 in co-culture with T47D breast cancer cells where the population has been doped with 5%, 10% or 30% T47D cells that had been transfected with even more MUC1*. FIG. 213B shows IL-18 secretion from huMNC2-CAR44 T cells with NFAT inducible IL-18 in co-culture with non-cancerous, MUC1 negative HEK293 cells where the cell population has been doped with 5%, 10% or 30% T47D cells that had been transfected with more MUC1*. As can be seen in the figure, the amount of IL-18 secreted into the media can be attributed to the MUC1* positive cells that the population was doped with. Time course fluorescent photographs of the experiment show that even when doped with significant percentages of high antigen density MUC1* positive cells, the MUC1 negative cells are not killed by the MUC1* targeting CAR T cells. FIG. 214A-214X shows photographs of T47D breast cancer cells (red) or non-cancerous HEK293 cells (also red), where both cell types have been doped with varying percentages of T47D cells engineered to express more MUC1* (green). These target cancer cells have been co-cultured with huMNC2-CAR44 T cells with NFAT inducible IL-18 wherein the IL-18 gene has been inserted into the Foxp3 enhancer/promoter. FIG. 214A-214F shows either T47D cells or HEK293 cells that have not been doped with T47D cells engineered to express high MUC1* density. FIG. 214G-214L shows either T47D cells or HEK293 cells that have been doped with 5% T47D cells engineered to express high MUC1* density. FIG. 214M-214R shows either T47D cells or HEK293 cells that have been doped with 10% T47D cells engineered to express high MUC1* density. FIG. 2145-214X shows either T47D cells or HEK293 cells that have been doped with 30% T47D cells engineered to express high MUC1* density. FIGS. 214A-B, G-H, M-N, and S-T show T47D breast cancer cells. FIGS. 214C-F, I-L, O-R, and U-X show HEK293 cells. As can be seen in the figures, the induced secretion of IL-18 resulted in low MUC1* density T47D cells being killed but did not induce non-specific killing of the MUC1* negative HEK293 cells. Taken together these results show that the Foxp3 system is a superior system for the inducible expression of a second factor and especially useful in CAR T systems. Further we have demonstrated the unexpected result that IL-18 increases the killing of low antigen density cells without the unwanted effect of killing nearby MUC1/MUC1* negative cells.

In another aspect, the invention is directed to a composition that includes at least two different plasmids transfected into the same immune cell, wherein the first encodes a CAR comprising an antibody fragment, scFv or peptide that binds to the extra cellular domain of an antigen on the surface of a B cell and the other encodes a gene that is not a CAR, wherein the gene that is not a CAR is expressed from an inducible promoter that is activated by elements of an activated immune cell. In one aspect, the immune cell is a T cell or an NK cell. In one aspect the immune cell is derived from a stem cell that has been directed to differentiate to that immune cell type in vitro. In another aspect, a CAR containing sequences of the antibody are expressed in a stem cell, which then may be differentiated into an immune cell. In one aspect the CAR comprises an antibody fragment, scFv or peptide that binds to CD19. In another aspect the antibody fragment, scFv or peptide binds to a surface antigen of a B cell or a B cell precursor, or binds to CD19, CD20, CD22, BCMA, CD30, CD138, CD123, CD33 or LeY antigen. In one aspect the non-CAR species is a cleavage enzyme. In another aspect the non-CAR species is a cytokine. In one aspect, the Cytokine is IL-7. In one aspect the cytokine is IL-15. In another aspect the cytokine is IL-7 and IL-15. In one case expression of the non-CAR species is induced by elements of an activated immune cell. In one aspect the element of an activated immune cell is an NFAT. In one aspect the NFAT is NFATc1, NFATc3 or NFATc2. that is not a CAR, wherein the gene that is not a CAR is expressed from an inducible promoter wherein expression is induced by elements of an activated immune cell. In one aspect the immune cell transfected or transduced with the composition is administered to a patient for the treatment or prevention of cancer. In one case the cancer is a leukemia, lymphoma or blood cancer.

It is not intended for the invention to be limited by a specific method or technology for inserting the gene or plasmid comprising a sequence encoding a CAR or activated T cell inducible protein or peptide there encoded. For example, the gene encoding the CARs and activated T cell induced genes described herein can be virally transduced into an immune cell using viruses, which may or may not result in the CAR gene being integrated into the genome of the recipient cell. Virus delivery systems and viral vectors include but are not limited to retroviruses, including gamma-retroviruses, lentivirus, adenoviruses, adeno-associated viruses, baculoviruses, poxvirus, herpes simplex viruses, oncolytic viruses, HF10, T-Vec and the like. In addition to viral transduction, CARs and activated T cell induced genes described herein can be directly spliced into the genome of the recipient cell using methods such as CRISPR technology, CRISPR-Cas9 and -CPF1, TALEN, Sleeping Beauty transposon system, and SB 100×.

Bulky cell surface proteins such as MUC1-FL can also cause a steric hindrance problem for BiTEs. A BiTE is a two-headed bispecific antibody wherein one head binds to a T cell and the other head binds to a tumor-associated antigen. In this way, the BiTE links together the T cell and the tumor cells. The antibody that binds to the T cell should be an antibody that activates the T cell, such as an antibody against CD3 or CD28. To solve the steric hindrance problem, the linker between the T cell specific antibody and the tumor specific antibody is lengthened.

In another aspect of the invention, an anti-MUC1* single chain molecule is fused to a cleavage enzyme or a catalytically active fragment of a cleavage enzyme. In one aspect of the invention, the cleavage enzyme is MMP9 (SEQ ID NO:643). In another aspect of the invention, the enzyme is a catalytically active fragment of MMP9 (SEQ ID NO:645). In some cases, the antibody fragment of the CAR is chosen for its ability to recognize MUC1* when cleaved by that specific cleavage enzyme. In one embodiment, the cleavage enzyme is MMP9, MMP3, MMP14, MMP2, ADAM17, ADAM TS16, and/or ADAM28. In one embodiment, the antibody or antibody fragment binds to a peptide having the sequence of (PSMGFR) GTINVHDVETQFNQYKTEAASRYNLTISDVSVSDVPFPFSAQSGA, PSMGFR N-10, QFNQYKTEAASRYNLTISDVSVSDVPFPFSAQSGA, or PSMGFR N+20 SNIKFRPGSVVVQLTLAFREGTINVHDVETQFNQYKTEAASRYNLTISDVSVSDVPFP FSAQSGA. In another embodiment, cleavage enzymes MMP9 and MMP3 are transduced into a T cell that is also transduced with a CAR with an antibody fragment that is a fragment of MNC2.

In many cases it is desirable to have the cleavage enzyme expressed only after an immune cell recognizes the tumor-associated target on a solid tumor. In this way, the cleavage enzyme will not freely move throughout the body, cleaving MUC1, MUC16 or other proteins, wherein their cleavage could actually promote cancer. However, there are cancers that are physically accessible to direct application of chemotherapy agents, CAR T cells and other anti-cancer agents. For example, types of brain cancers, prostate cancer and ovarian cancers have all shown the benefit of direct application of anti-cancer agents into the local vicinity of the cancer. CAR T cells have been injected directly into the brain and/or cerebral spinal fluid of glioblastoma patients. Radiation has been directed to the prostate area for the treatment of prostate cancers, including those that have metastasized. Hot chemo therapy agents have been directly injected into the intraperitoneal cavity for the treatment of ovarian cancers. In these and other cases, where the cancers that are physically accessible to direct application of chemotherapy agents, a cleavage enzyme is administered in the presence or absence of another anti-cancer agent, which could be a CAR T cell, an immune cell engineered to recognize a tumor-associated antigen, a BiTE, an ADC, a biological or a standard chemotherapy agent. Although ovarian cancer can metastasize to anywhere in the body, it usually stays in the abdomen as it spreads to adjacent organs, such as the intestines, liver and stomach. This makes ovarian cancer an ideal test case for improving the effect of anti-cancer agents by administering a cleavage enzyme in combination with other anti-cancer agents, including a platinum-based drug such as carboplatin (Paraplatin) or cisplatin, and/or a taxane such as paclitaxel (Taxol) or docetaxel (Taxotere). Alkeran (Melphalan), Avastin (Bevacizumab), Carboplatin, Clafen (Cyclophosphamide), and Cytoxan have all been approved for the treatment of ovarian cancer. Other treatments that are being tested for the treatment of ovarian cancers include agents that target MUC1, MUC16 and as described herein, MUC1*.

Other cleavage enzymes can be used in addition to or in place of MMP9. MMP14 for example, has been shown to efficiently cleave MUC1 to MUC1* (FIG. 38). In one aspect of the invention, MMP14 is expressed in an immune cell that is also engineered to express a CAR. In one case the CAR is an anti-MUC1* CAR. For example, it can be an MNC2-CAR44 transduced T cell. In another aspect of the invention, the MMP14 is directly administered to the patient either in the location of the tumor or by i.v.

In yet another aspect of the invention, the cancer is an ovarian cancer and either MMP9 or MMP14 is directly injected into the abdominal area along with an anti-cancer agent, which can be a chemotherapy agent, a biological, an anti-MUC1* CAR T or an anti-MUC16 CAR T.

In addition to local administration of the cleavage enzyme, + iv administration alone or secreted from an immune cell, which may be a CAR T cell, which further may be expressed off of an inducible promoter is contemplated.

### Methods Used in Carrying Out Experimentation in Relation to the Present Invention

### 1. Lentivirus Production and Viral Transduction of Immune Cells

HEK293 or HEK293T cells (ATCC) were used to produce lentivirus. The day prior transfection plates (6well plate) were coated with poly-D-lysine and cells seeded so that cell density reaches 90-95% at the time of transfection and cultures in a 5% CO₂ atmosphere. The next day cells were transfected with Lipofectamine 3000 (life technologies) and Opti-MEM® I Reduced Serum Medium according to the manufacturer instructions (0.75 ug of lentiviral expression vector and 2.25 ug of pPACKH1 packaging mix was used). After 6 h incubation, the media was changed and media containing lentivirus was harvested after 24 and 48 hours. Lentivirus was concentrated with Lenti-X concentrator (Clontech) and titer was calculated using the Lenti-X p@4 Rapid Titer Kit (Clontech). Lentivirus was store at −80 C in single-use aliquots.

Transduction of Immune Cells with Constructs Including CARs

Human T cells, if frozen, were thawed and pre-warmed in 100-200 units IL-2 and TexMACS medium, 20 ml, and pelleted by centrifugation. Cells were resuspended in 10 ml of medium and cultured at 37° C., 5% CO₂ at 1×10⁶ cells/ml in complete medium with anti-CD3/anti-CD28 beads (TransAct kit).

After 4 days in culture, cells were counted and 450 ul of cell suspension was placed in single well of a 24-well plate at a density of approximately 1×10⁶ cells/ml. Cells were allowed to settle. 150 ul was carefully removed from the top of each well. To each well was added an appropriate dilution of lentiviral vector, diluted in plain TexMACS medium, along with protamine sulfate to a final concentration of 10 ug/ml, in a 150 ul volume, for a final total volume of 450 ul per well and incubated for 24 hrs. Transduced cells were removed, pelleted by centrifugation, and resuspended in fresh medium, adjusting cell density, not to exceed 1.0×10⁶ cells/ml. Transduced T cells can be expanded and frozen or used directly. Typically transduced T cells are used or frozen between Day 7 and Day 20 post activation with IL-2 and TransAct media.

### 2. Comparing Anti-MUC1* CAR T Cell Activity in the Presence or Absence of Exogenous Cleavage Enzymes

Human T cells (ALLCELLS) were transduced with huMNC2-CAR44 or huMNC2-CAR50. CAR44 is huMNC2-scFv-CD8-CD8 (transmembrane-41BB-3z). CAR50 is the same as CAR44 except that CAR50 has a murine MNC2-scFv and a CD4 transmembrane domain. The CAR T cells were incubated for 18 hours with target and non-target cells that have been dyed red using CMTMR. When T cells recognize a target cell, they cluster the target cells and begin to kill them. As can be seen in FIGS. 45-47 the CAR T cells effectively cluster and kill the target MUC1* positive cancer cells. FIG. 45 shows huMNC2-CAR44 or huMNC2-CAR50 T cells being co-cultured with HCT-116 cells transduced to express MUC1*, “HCT-MUC1*” or with HCT-116 cells transduced with a full-length MUC1, “HCT-MUC1-41TR”. Recall that MNC2 recognizes an ectopic epitope that is only revealed after cleavage and release of the MUC1 tandem repeat domain. Neither huMNC2-CAR44 nor huMNC2-CAR50 T cells recognize the cells expressing full-length MUC1 (FIG. 45F-45H). However, when MMP9 plus activator APMA is added, the CAR T cells recognize the cells, cluster and kill them (FIG. 45J-45L). The addition of cleavage enzyme ADAM-17 did not affect the recognition of either CAR T cell for full-length MUC1 (FIG. 45N-45P). The reason could be that ADAM-17 doesn't cleave MUC1 or the cleavage product is not recognized by MNC2. A similar experiment was performed (FIG. 46) that showed that MMP2 was only weakly effective at either cleavage MUC1 or that the MMP2 cleavage product was only weakly recognized by MNC2. FIG. 47 shows the contrast between huMNC2-CAR44 recognition of HCT-MUC1* cells, T47D-wt breast cancer cells, and T47D cells with added MMP9 which presumably cleaves the full-length MUC1 to an MNC2 recognizable MUC1*.

### 3. Confocal Imaging of CAR T Cells Giving the “Kiss of Death” to MUC1* Positive Cancer Cells

Confocal images of Human T cells that were transduced with huMNC2-CAR44, co-cultured for 24 hours with MUC1* positive DU145 prostate cancer cells showed the CAR T cells inserting Granzyme B into the target cancer cells. FIG. 55 shows fluorescent images of the huMNC2-CAR44 T cells secreting Granzyme B when co-cultured with the prostate cancer cells, FACS analysis showing increased expression of Granzyme B by the CAR T cells and an xCELLigence experiment showing that the target prostate cancer cells were in fact killed.

### 5. Analysis of CAR T Cell Induced Killing of MUC1* Positive Cancer Cells by FACS Analysis

We have demonstrated the killing effect of huMNC2-CAR44 T cells on T47D MUC1* positive breast cancer cells, wherein the breast cancer cells have been transfected with increasing amounts of additional MUC1*. The killing effect of the huMNC2-CAR44 T cells increases as the amount of target MUC1* expressed on the cells increases.

IFN-γ secretion in media was measured using a human IFN-γ ELISA kit (Biolegend). Plates were coated with an anti-IFN-γ antibody (capture antibody, 1× in coating buffer). After overnight incubation at 4° C., the plate was washed 4 times with PBS-T and blocking solution was added to block remaining binding site on the well. After 1 h at RT (shaking at 500 rpm) the plate was washed 4 times with PBS-T and conditioned media (CM) and IFN-γ standard, was added. After 2 h at RT with shaking, the plate was washed 4 times with PBS-T and detection antibody (1×), was added. After 1 h at RT with shaking, the plate was washed 4 times with PBS-T and Avidin-HRP (1×) was added. After 30 min at RT with shaking, the plate was washed 5 times with PBS-T (soak 1 min each wash) and TMB substrate solution was added. The reaction was stopped after 20 min by adding the stop solution and absorbance was read at 450 nm (minus absorbance at 570 nm) within 15 min of stopping.

### 6. Analysis of CAR T Cell Induced Killing of MUC1* Positive Cancer Cells by xCELLigence

In addition to FACS analysis, many researchers now use an xCELLigence instrument to measure CAR T killing of cancer cells. The xCELLigence instrument uses electrode arrays upon which cancer cells are plated. The adherent cancer cells insulate the electrode and so cause an increase in impedance as they grow. Conversely, T cells are not adherent and remain in suspension so do not contribute to insulation of the electrode which would increase impedance. However, if the T cells or CAR T cells kill the cancer cells on the electrode plate, the cancer cells ball up and float off as they die, which causes the impedance to decrease. The xCELLigence instrument measures impedance as a function of time, which is correlated to cancer cell killing. In addition, the electrode plates also have a viewing window. When CAR T cells effectively kill the adsorbed target cancer cells, there is a decrease in impedance but also one can see that there are no cancer cells left on the plate surface.

In most of the XCELLigence experiments, 5,000 cancer cells were plated per well of a 96-well electrode array plate. Cells were allowed to adhere and grow for 24 hours. CAR T cells were then added at an Effector to Target ratio (E:T) of 0.5:1, 1:1, 2:1, 5:1, 10:1 and sometimes 20:1. The E:T ratio assumes 100% transduction of the CAR into the T cells, when the actual transduction efficiency is 40%.

The xCELLigence instrument records impedance as a function of time and experiments can go on for up to 7 days.

FIG. 48, FIG. 49, FIG. 55H, FIG. 56H, FIGS. 57A-57C, all show results of CAR T and cancer cell experiments performed on an xCELLigence instrument.

### 7. Anti-MUC1* CAR T Cell Therapy in Mice Bearing Human Tumors

Female NOD/SCID/GAMMA (NSG) mice between 8-12 weeks of age were implanted with 500,000 human cancer cells, wherein the cancer cells had previously been stably transfected with Luciferase. Mice bearing Luciferase positive cells can be injected with the enzyme's substrate Luciferin just prior to imaging, which makes the cancer cells fluoresce. The cancer cells are imaged in live mice within 10-15 minutes after injection with Luciferin on an IVIS instrument. The readout is flux or photons per second. Tumors were allowed to engraft until tumors were clearly visible by IVIS.

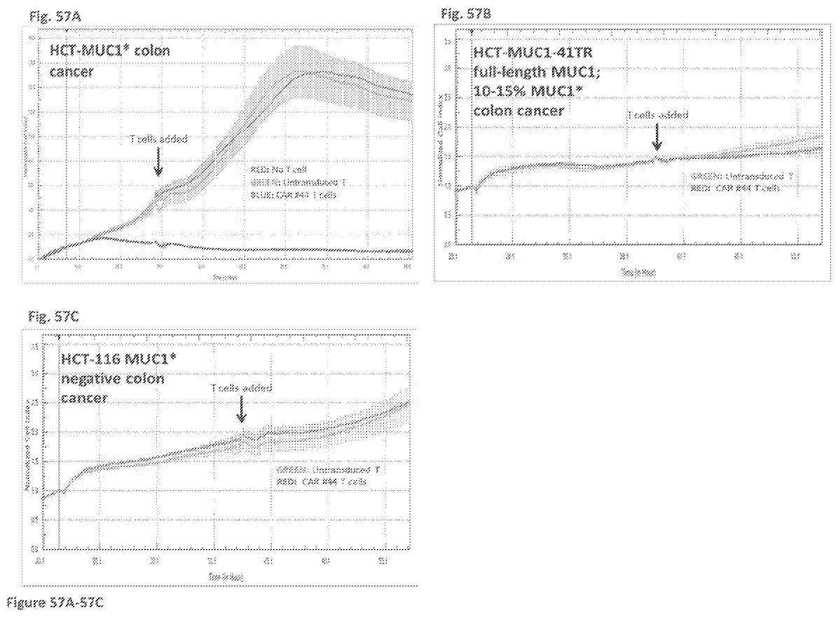

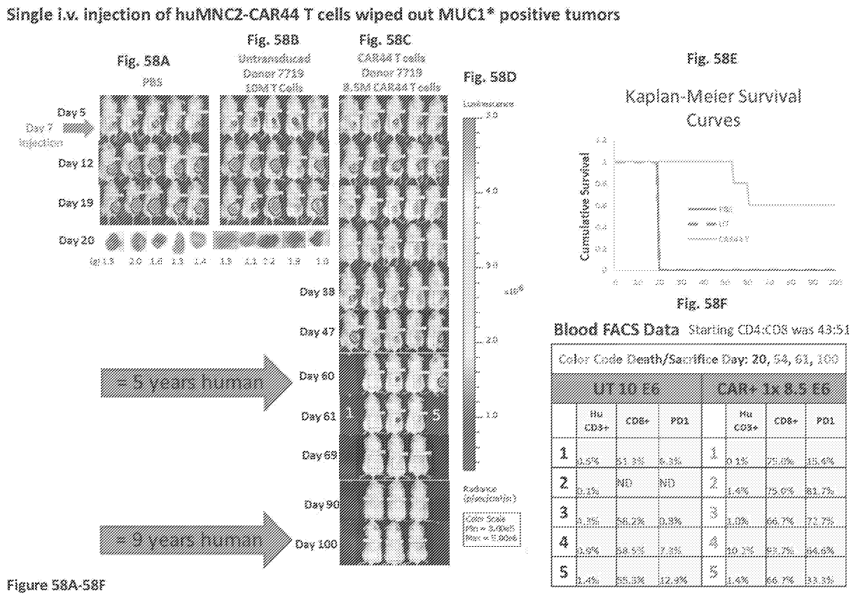

FIGS. 58A-58F show fluorescent photographs of mice taken on an IVIS instrument. 10 minutes prior to IVIS photographs, mice were injected intraperitoneally (IP) with Luciferin, which fluoresces after cleavage by Luciferase, thus making tumor cells fluoresce. NSG (NOD/SCID/GAMMA) immune compromised mice that on Day 0 were subcutaneously implanted on the flank with 500,000 human MUC1* positive cancer cells that had been stably transfected with Luciferase. Tumors were allowed to engraft. On Day 7 after IVIS measurement, animals were tail vein injected with either PBS, 10 million untransduced human T cells or 8.5M huMNC2-scFv-CAR44 T cells. As can be seen in the figure, control mice had to be sacrificed on Day 20 due to excess tumor burden (FIG. 58A-58B). huMNC2-CAR44 T cell treated mice were tumor free after a single CAR T cell injection until Day 100 when they were sacrificed (FIG. 58C). FIG. 58E shows Kaplan-Meier survival curves that demonstrate the efficacy of T cell therapy guided by anti-MUC1* antibody. FIG. 58F shows a table summarizing the characteristics of the human T cells that were collected from the test mice upon sacrifice. The starting Car T cell population was 50% CD4 positive helper T cells and 50% CD8 positive killer T cells. As can be seen in the table, the percent of CD8 positive cells has increased in the CAR T treated group, indicating in vivo expansion of that group of cells, which is an indicator of efficacy. We also note that in the treated group, the CAR T cells express higher levels of PD1 which is a marker of T cell exhaustion.

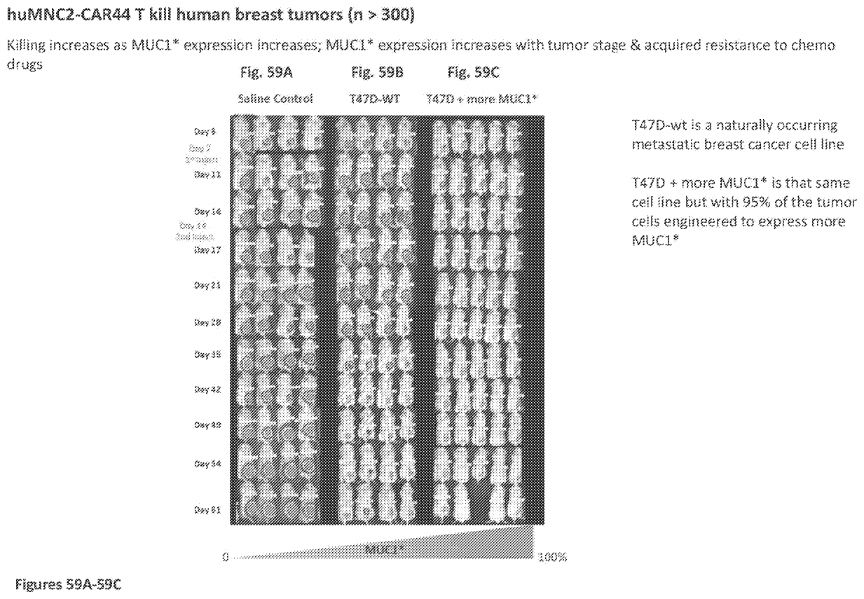

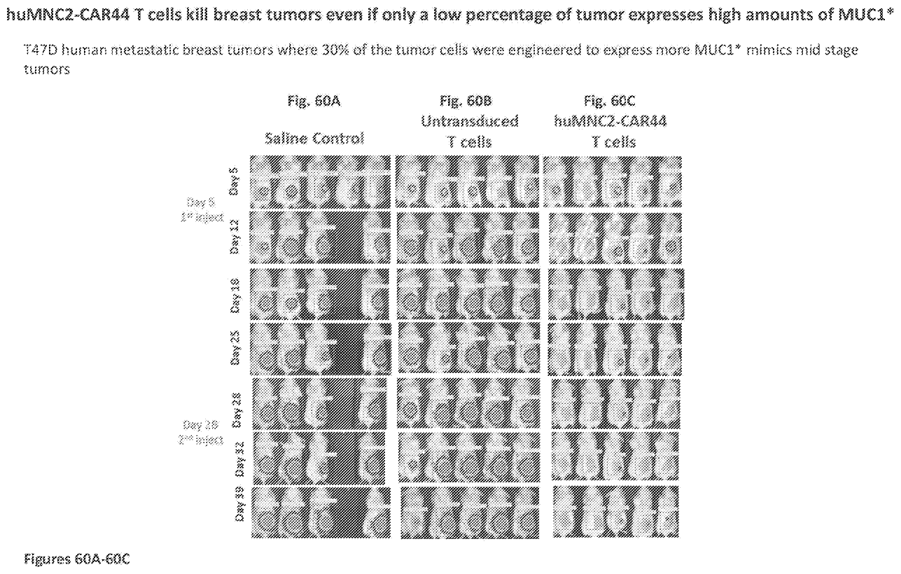

In another animal experiment, NSG mice were sub-cutaneously implanted into the flank with 500,000 tumor cells then injected on Day 7 and again on Day 14 with either saline solution, PBS, or 10M huMNC2-CAR44 T cells (FIG. 59A-59C). In this experiment the amount of MUC1* expressed on the tumor cells was varied. In one case, the tumor cells that were implanted were T47D-wildtype (FIG. 59B). In another case, the T47D cells were doped with 95% T47D cells that had been transfected to express even more MUC1* (FIG. 59C). As can be seen, the tumors comprised of cells expressing more MUC1* were eliminated more quickly and did not recur. In a similar experiment, the tumor cells were doped with a relatively small amount of cells that expressed more MUC1*. FIG. 60A-60C shows NSG mice implanted with T47D-wt breast cancer cells that have been doped with 30% of T47D cells transfected to express more MUC1*. As can be seen, even a small percentage of cells expressing high levels of MUC1* is sufficient to trigger CAR T cell mediated killing of the entire tumor. Naturally occurring tumors are heterogeneous and are comprised of both high and low antigen expressing cells. This experiment indicates that huMNC2-CAR44 T cells would be effective in eradicating naturally occurring tumors.

FIGS. 61A-61J show fluorescent photographs of mice taken on an IVIS instrument. NSG (NOD/SCID/GAMMA) immune compromised mice that on Day 0 were subcutaneously injected into the flank with 500K human BT-20 cells which are a MUC1* positive triple negative breast cancer cell line. The cancer cells had been stably transfected with Luciferase. Tumors were allowed to engraft. On Day 6 after IVIS measurement, animals were given a one-time injection of 10 million of either human T cells transduced with huMNC2-scFv-CAR44 or untransduced T cells. 5 million T cells were injected intra-tumor and 5 million were injected into the tail vein. 10 minutes prior to IVIS photographs, mice were IP injected with Luciferin. In one case the huMNC2-CAR44 T cells were first incubated with beads to which was attached the PSMGFR peptide to pre-stimulate the T cells and in the figure is marked Protocol 1. In Protocol 2, the huMNC2-CAR44 T cells were pre-stimulated with live tumor cells, which likely injected more tumor cells into the animals' circulation.

FIGS. 62A-62M show fluorescent photographs of mice taken on an IVIS instrument. NSG (NOD/SCID/GAMMA) immune compromised mice that on Day 0 were injected into the intraperitoneal cavity (IP) with 500K human SKOV-3 cells which are a MUC1* positive ovarian cancer cell line. The cancer cells had been stably transfected with Luciferase. Tumors were allowed to engraft. On Day 3 after IVIS measurement, animals were IP injected with 10M either human T cells transduced with huMNC2-CAR44 T cells, untransduced T cells or PBS. Animals were IVIS imaged again on Day 7. 10 minutes prior to IVIS photographs, mice were IP injected with Luciferin. As can be seen in the figure the anti-MUC1* CAR T cells effectively reduced ovarian tumor volume by Day 15.

### 9. NFAT-Induced IL-18 Sequences and Cloning

Cloning of IL18 in pGL4-14 3×NFAT:

An activated IL18 (SEQ ID NO:1644) was synthesized with the CD8 leader sequence. The pGL4-14 3×IL2 NFAT and pGL4-14 3×FoxP3 NFAT were digested with XhoI and HindIII restriction enzymes (New England Biolabs). The purified plasmids and the synthesized IL18 sequences were assembled using the Gibson assembly cloning kit (New England Biolab). The resulting constructs (pGL4-14 3×IL2NFAT-IL18 and pGL4-14 3×FoxP3NFAT-IL18) contains 3 repeats of NFAT response element (IL2 or FoxP3) followed by a minimum promoter (mCMV: SEQ ID NO:1634) and IL18 (SEQ ID NOS:1752-1753) with CD8 leader sequence.

Cloning of MNC2 CAR with IL18 in pCDNA vector:

MNC2 CAR sequence was amplified from previously made vector by polymerase chain reaction (PCR) using the following primers: 5′-agggagacccaagctggctagttaagcttggatggccttaccagtgaccgccttgc-3′ (SEQ ID NO:1754) and 5′-taggccagagaaatgttctggcattatcagcgagggggcagggcctgc-3′ (SEQ ID NO:1755).

IL18 sequence including NFAT response element was amplify from pGL4-14 3×NFAT-IL18 by polymerase chain reaction (PCR) using the following primers: 5′-tgccagaacatttctctgg-3′ (SEQ ID NO: 1756) and 5′-acagtcgaggctgatcagcgggtttaaacttatcagtcctcgttctgcacgg-3′ (SEQ ID NO: 1757). The purified PCR fragments and digested pCDNA 3.1 V5 (ThermoFisher scientific) were assembled using the Gibson assembly cloning kit (New England Biolab) to create the construct pCDNA MNC2CAR-3×IL2NFAT-IL18 and pCDNA MNC2CAR-3×FoxP3NFAT-IL18.

### Cloning of MNC2 CAR-NFAT-IL18 in Lentivector:

MNC2 CAR-NFAT-IL18 sequence was amplified from pCDNA MNC2CAR-3×IL2NFAT-IL18 and pCDNA MNC2CAR-3×FoxP3NFAT-IL18.by polymerase chain reaction (PCR) using the following primers: 5′-atgcaggccctgccccctcgctgataagtttaaactgccagaacatttctctggcctaac-3′ (SEQ ID NO:1758) and 5′-accggagcgatcgcagatccttcgcggccgcttatcagtcctcgttctgcacggtgaac-3′ (SEQ ID NO:1759). The purified PCR fragments and digested pCDH Dual Hygro (System Biosciences, CA) were assembled using the Gibson assembly cloning kit (New England Biolab) to create the construct pCDH MNC2CAR-3×IL2NFAT-IL18 and pCDH MNC2CAR-3×FoxP3NFAT-IL18.

Creation of Lentivector with MSCV Promoter

MSCV promoter sequence was amplified from pCDH-MSCV-MCS-EF1a-GFP (System Biosciences).by polymerase chain reaction (PCR) using the following primers: 5′-attgcactagttgaaagaccccacctgtagg-3′ (SED ID NO:1760) and 5′-aatgctctagaatacgggtatccagg-3′ (SEQ ID NO:1761). After digestion with Spel and XbaI restriction enzymes (New England Biolabs), the purified fragment was cloned into pCDH CMV MCS (System Bioscience) digested with the same restriction enzymes to create the construct pCDH MSCV MCS.

Cloning of MNC2 CAR-NFAT-IL18 in pCDH MSCV MCS:

MNC2 CAR-IL2NFAT-IL18 sequence was amplified from pCDNA MNC2CAR-3×IL2NFAT-IL18 by polymerase chain reaction (PCR) using the following primers: 5′ atagcgaattcgtaccgagggccaccatgg-3′ (SEQ ID NO:1762) and 5′-taggcctcccaccgtacacgcctaggtaccacgccttctgtatg-3′ (SEQ ID NO:1763) MNC2 CAR-IL2NFAT-IL18 sequence was amplified from pCDNA MNC2CAR-3×FoxP3NFAT-IL18 by polymerase chain reaction (PCR) using the following primers: 5′ atagcgaattcgtaccgagggccaccatgg -3′ (SEQ ID NO:1762) and 5′-taggcctcccaccgtacacgcctaggtacctctgcagtaaatgg-3′ (SEQ ID NO: 1764). After digestion with EcoRI and KpnI restriction enzymes (New England Biolabs), the purified fragment was cloned into pCDH MSCV MCS digested with the same restriction enzymes to create the construct pCDH MSCV MNC2CAR-3×IL2NFAT-IL18 and pCDH MSCV MNC2CAR-3×FoxP3NFAT-IL18.

### Cloning of 6×NFAT Response Elements:

6×NFAT (IL2 and FoxP3) response element were synthesized followed by different minimal promoter: mCMV (SEQ ID NO:1634), mIL2P (SEQ ID NO: 1635) and miniP (SEQ ID NO:1636). A total of six 6 sequences were synthesized: SEQ ID NOS: 1768-1779.

6×NFAT sequences were amplified by polymerase chain reaction (PCR) using the following primers: 5′-tgccagaacatttctctgg-3′ (SEQ ID NO:1756) and 5′-taaggccatggtggctagc-3′ (SEQ ID NO:1765). The purified PCR fragments and digested (KpnI and XhoI) pCDNA MNC2CAR 3×NFAT IL18 were assembled using the Gibson assembly cloning kit (New England Biolab) to create constructs with 6× NFAT response elements in place of the 3× NFAT response elements.

6×NFAT sequences were amplified, from the pCDNA vector created above, by polymerase chain reaction (PCR) using the following primers: 5′-aataagtttaaactgccagaacatttctctgg-3′ (SEQ ID NO: 1766) and 5′-atatagcggccgcttatcagtcctcgttctgcacgg-3′ (SEQ ID NO:1767). After digestion with PmeI and NotI restriction enzymes (New England Biolabs), the purified fragments were cloned into pCDH MSCV MNC2CAR digested with the same restriction enzymes to create the construct pCDH MSCV MNC2CAR-6×IL2NFAT-IL18 and pCDH MSCV MNC2CAR-6×FoxP3NFAT-IL18. For each construct 3 minimal promoter were tested.

Those skilled in the art will recognize, or be able to ascertain using no more than routine experimentation, many equivalents to the specific embodiments of the invention specifically described herein.

1.-71. (canceled)

72. A chimeric antigen receptor (CAR), comprising:
(a) an anti-Mucin 1* (MUC1*) antibody comprising:
(i) a heavy chain (HC) variable region comprising a complementarity determining region 1 (HC-CDR1) comprising the amino acid sequence of SEQ ID NO: 123, a HC-CDR2 comprising the amino acid sequence of SEQ ID NO: 127, and a HC-CDR3 comprising the amino acid sequence of SEQ ID NO: 131; and
(ii) a light chain (LC) variable region comprising a complementarity determining region 1 (LC-CDR1) comprising the amino acid sequence of SEQ ID NO: 173, a LC-CDR2 comprising the amino acid sequence of SEQ ID NO: 177, and a LC-CDR3 comprising the amino acid sequence of SEQ ID NO: 181; and
(b) a cytoplasmic region comprising a CD3 zeta intracellular signaling domain comprising a mutation in a first immunoreceptor tyrosine-based activation motif (ITAM),
wherein:
when the CAR is expressed on a surface of an immune cell,
the CAR binds specifically to a cancer cell, and
the CAR has reduced signaling as compared to the same CAR but without the mutation in the first ITAM.

73. The CAR of claim 72, wherein:
(a) the heavy chain variable region comprises an amino acid sequence with at least 90%, sequence identity to the amino acid sequence of SEQ ID NO: 145; and
(b) the light chain variable region comprises an amino acid sequence with at least 90% sequence identity to the amino acid sequence of SEQ ID NO: 195.

74. The CAR of claim 72, wherein:
(a) the heavy chain variable region comprises the amino acid sequence of SEQ ID NO: 145; and
(b) the light chain variable region comprises the amino acid sequence of SEQ ID NO: 195.

75. The CAR of claim 72, wherein the antibody comprises an amino acid sequence with at least 90% sequence identity to the amino acid sequence of SEQ ID NO: 239.

76. The CAR of claim 72, wherein the antibody comprises the amino acid sequence of SEQ ID NO: 239.

77. The CAR of claim 72, wherein the mutation in the first ITAM comprises a point mutation at a tyrosine residue.

78. The CAR of claim 77, wherein the tyrosine residue is mutated to a phenylalanine residue.

79. The CAR of claim 78, wherein the CD3 zeta intracellular signaling domain comprises:
the first ITAM comprising a substitution of a tyrosine residue with a phenylalanine residue,
a second ITAM comprising a substitution of a tyrosine residue with a phenylalanine residue, and
a third ITAM that does not comprise a point mutation.

80. The CAR of claim 79, wherein the cytoplasmic domain of the CAR comprises, in an N-terminal to C-terminal orientation, an ITAM1, an ITAM2, and an ITAM3, wherein the first ITAM is the ITAM2 and the second ITAM is the ITAM3.

81. The CAR of claim 79, wherein the CD3 zeta intracellular signaling domain comprises an amino acid sequence with at least 90% sequence identity to the amino acid sequence of SEQ ID NO: 1797.

82. The CAR of claim 79, wherein the CD3 zeta intracellular signaling domain comprises the amino acid sequence of SEQ ID NO: 1797.

83. The CAR of claim 72, wherein the cytoplasmic region comprises a co-stimulatory domain comprising a sequence derived from CD27, CD28, 4-1BB, OX40, CD30, CD40, ICAM-1, LFA-1, ICOS, CD2, CD5, CD7, Fc receptor gamma domain, or combinations thereof.

84. The CAR of claim 83, wherein the co-stimulatory domain comprises an amino acid sequence with at least 95% sequence identity to the amino acid sequence of SEQ ID NO: 378, or 380.

85. The CAR of claim 83, wherein the co-stimulatory domain comprises the amino acid sequence of SEQ ID NO: 378.

86. The CAR of claim 72, wherein the CAR further comprises a transmembrane domain.

87. The CAR of claim 86, wherein the transmembrane domain comprises a sequence derived from CD4, CD8, CD28, 4-1BB, or OX40, or combinations thereof.

88. The CAR of claim 86, wherein the transmembrane domain comprises an amino acid sequence with at least 95% sequence identity to the amino acid sequence of SEQ ID NO: 364, 366, 368, 370, or 372.

89. The CAR of claim 86, wherein the transmembrane domain comprises the amino acid sequence of SEQ ID NO: 368.

90. The CAR of claim 72, wherein the CAR further comprises a hinge domain C-terminal to the antibody.

91. The CAR of claim 90, wherein the hinge domain comprises a sequence derived from CD4, CD8, or C28.

92. The CAR of claim 90, wherein the hinge domain comprises an amino acid sequence with at least 90% sequence identity to the amino acid sequence of SEQ ID NO: 346, 348, or 350.

93. The CAR of claim 90, wherein the hinge domain comprises the amino acid sequence of SEQ ID NO: 350.

94. The CAR of claim 72, wherein the CAR further comprises a hinge domain, a transmembrane domain, and a co-stimulatory domain, wherein the CAR has an N-terminal to C-terminal arrangement of the anti-MUC1* antibody, the hinge domain, the transmembrane domain, the co-stimulatory domain, and the CD3 zeta intracellular signaling domain.

95. The CAR of claim 94, wherein:
(a) the anti-MUC1* antibody comprises an amino acid sequence with at least 90% sequence identity to SEQ ID NO: 239;
(b) the co-stimulatory domain comprises an amino acid sequence with at least 95% sequence identity to SEQ ID NO: 378; and
(c) the CD3 zeta intracellular signaling domain comprises an amino acid sequence with at least 95% sequence identity to SEQ ID NO: 1797, wherein the mutation in the first ITAM comprises two point mutations at two tyrosine residues, wherein the two tyrosine residues in the first ITAM are mutated to two phenylalanine residues, wherein the CD3 zeta intracellular signaling domain further comprises a mutation in a second ITAM, wherein the mutation in the second ITAM comprises two point mutations at two tyrosine residues, and wherein the two tyrosine residues in the second ITAM are mutated to two phenylalanine residues.

96. The CAR of claim 94, wherein:
(a) the anti-MUC1* antibody comprises the amino acid sequence of SEQ ID NO: 239;
(b) the hinge domain comprises the amino acid sequence of SEQ ID NO: 350;
(c) the transmembrane domain comprises the amino acid sequence of SEQ ID NO: 368;
(d) the co-stimulatory domain comprises the amino acid sequence of SEQ ID NO: 378; and
(e) the CD3 zeta intracellular signaling domain comprises the amino acid sequence of SEQ ID NO: 1797.

97. The CAR of claim 72, wherein the CAR comprises an amino acid sequence with at least 90% sequence identity to the amino acid sequence of SEQ ID NO: 1785, wherein the mutation in the first ITAM comprises two point mutations at two tyrosine residues, wherein the two tyrosine residues in the first ITAM are mutated to two phenylalanine residues, wherein the CD3 zeta intracellular signaling domain further comprises a mutation in a second ITAM, wherein the mutation in the second ITAM comprises two point mutations at two tyrosine residues, and wherein the two tyrosine residues in the second ITAM are mutated to two phenylalanine residues.

98. The CAR of claim 72, wherein the CAR comprises the amino acid sequence of SEQ ID NO: 1785.

99. A vector comprising a nucleic acid sequence encoding the CAR of claim 72.

100. A viral vector comprising a nucleic acid sequence encoding the CAR of claim 72.

101. A lentiviral vector comprising a nucleic acid sequence encoding the CAR of claim 72.

102. A cell comprising the CAR of claim 72.

103. A cell comprising a nucleic acid sequence encoding the CAR of claim 72.

104. The cell of claim 102, wherein the cell is an immune cell.

105. The cell of claim 104, wherein the immune cell is a T cell, a natural killer (NK) cell, a dendritic cell, or a mast cell.

106. The cell of claim 104, wherein the immune cell is a T cell.

107. A method for treating a MUC1*-positive cancer, comprising administering to a subject in need thereof a therapeutically effective amount of the cell of claim 102.
